(19) United States
(12) Patent Application Publication
(10) Pub. No.: US 2026/0193268 A1
(43) Pub. Date: Jul. 9, 2026

(54) TRICYCLIC PI3Ka INHIBITOR, PREPARATION METHOD THEREFOR, AND PHARMACEUTICAL USE THEREOF

(71) Applicant: WIGEN BIOMEDICINE TECHNOLOGY (SHANGHAI) CO., LTD., Shanghai (CN)

(72) Inventors: Yuli XIE, Shanghai (CN); Yongcong LV, Shanghai (CN); Lihui QIAN, Shanghai (CN)

(21) Appl. No.: 19/130,500

(22) PCT Filed: Nov. 16, 2023
(86) PCT No.: PCT/CN2023/132092

§ 371 Date: May 15, 2025

(30) Foreign Application Priority Data
Nov. 17, 2022 (CN) 202211457330.4
May 15, 2023 (CN) 202310546509.5
Sep. 28, 2023 (CN) 202311275481.2

(51) Int. Cl. C07D 498/04; A61K 31/553; A61K 31/675; C07D 498/10; C07D 519/00; C07F 9/6561

(52) U.S. Cl. CPC C07D 498/04; A61K 31/553; A61K 31/675; C07D 498/10; C07D 519/00; C07F 9/6561

(57) ABSTRACT

Disclosed are a tricyclic PI3Kα inhibitor, a preparation method therefor, and pharmaceutical use thereof. Particularly, the present invention relates to a compound represented by general formula (1), a preparation method therefor, and use of the compound of general formula (1) and isomers thereof, crystal forms thereof, pharmaceutically acceptable salts thereof, hydrates thereof, or solvates thereof as PI3Kα inhibitors. The compound of the present invention and the isomers thereof, crystal forms thereof, pharmaceutically acceptable salts thereof, hydrates thereof, or solvates thereof can be used for preparing drugs for treating or preventing PI3Kα-mediated related diseases.

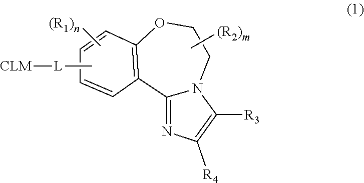

The present application claims priority to Chinese Patent Application No. 202211457330.4 filed on Nov. 17, 2022, Chinese Patent Application No. 202310546509.5 filed on May 15, 2023, and Chinese Patent Application No. 202311275481.2 filed on Sep. 28, 2023, which are incorporated herein by reference in their entirety.

### TECHNICAL FIELD

The present disclosure pertains to the field of pharmaceutical chemistry, and particularly to a novel compound with an inhibitory effect on phosphatidylinositol 3-kinase alpha (PI3Kα), a preparation method therefor, and use of such compounds in the preparation of anti-tumor drugs.

### BACKGROUND

The phosphatidylinositol-3-kinase (PI3K)-protein kinase B (Akt/PKB)-mammalian target of rapamycin (mTOR) pathway is a critical signaling pathway in mammalian tumor immunity, and it is closely associated with cell proliferation, cell cycle progression, cell survival, cell growth, angiogenesis, etc. Activation of this pathway is closely associated with tumor cell apoptosis and migration, as well as tumor occurrence, development, and drug resistance. The PI3K protein family is divided into four major classes (I, II, III, and IV), each with distinct structures and functions. Among them, class I PI3K is the most extensively studied and is further classified into four subtypes: PI3Kα, PI3Kβ, PI3Kδ, and PI3Kγ. PI3Kα exhibits activating mutations and amplifications in various tumors and is closely associated with tumor occurrence and development. PI3Kα is activated by receptor tyrosine kinases (RTKs) and G protein-coupled receptors (GPCRs). Upon activation, it catalyzes the conversion of phosphatidylinositol-4,5-bisphosphate (PIP2) to phosphatidylinositol-3,4,5-trisphosphate (PIP3). PIP3 further activates protein kinase B (Akt/PKB) and its downstream signaling pathways. PI3Kα consists of the p110α catalytic subunit and the p85 regulatory subunit. The Cancer Genome Atlas project analyzes mutations in over 3000 cancers and identifies PIK3CA, the gene encoding PI3Kα, as the second most frequently mutated oncogene. Hotspot mutations in PIK3CA primarily localize to the helical region and kinase domain, including E542K, 545K, and H1047R. The mutated PIK3CA not only aberrantly activates PI3Kα but also inhibits the expression of the tumor suppressor gene PTEN, leading to rapid cell proliferation and thus resulting in tumorigenesis. Therefore, PI3Kα remains a vital target in the development of various anti-cancer drugs.

PI3K inhibitors are mainly categorized into pan-PI3K inhibitors and selective PI3K inhibitors. Pan-PI3K inhibitors act on all subtype proteins but lack specificity for a single subtype, and thus have varying degrees of potential toxicity. Inhibition of PI3Kβ can lead to thrombocytopenia and thrombosis, inhibition of PI3Kδ can lead to immune system abnormalities, autoimmunity, and frequent infections, and inhibition of PI3Kγ, which is closely associated with blood pressure stability and smooth muscle contraction, can lead to hypertension. Therefore, the development of highly active and selective PI3Kα inhibitors can prevent off-target toxicity, demonstrating significant clinical application value.

Currently, only one selective PI3Kα inhibitor, Alpelisib (developed by Novartis), is commercially available. In 2019, it was approved for use in combination with fulvestrant to treat postmenopausal women and men with HR+/HER2− advanced or metastatic breast cancer harboring PIK3CA gene mutations, who experience disease progression during or after endocrine therapy. Additionally, several other selective PI3Kα inhibitors, such as GDC-0077 and CYH33, are under clinical research.

### SUMMARY

The present disclosure provides a compound of general formula (1) or an isomer thereof, a crystalline form thereof, a pharmaceutically acceptable salt thereof, a hydrate thereof, or a solvate thereof:

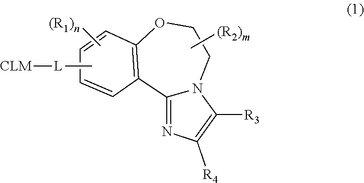

wherein in general formula (1):
CLM is a group that can covalently bind to a PI3Kα protein;
L is a group connecting CLM and a tricyclic structure;
R₁ and R₂ are independently selected from hydrogen, deuterium, alkyl, deuterated alkyl, haloalkyl, alkoxy, haloalkoxy, halogen, amino, carbamoyl, sulfydryl, nitro, hydroxy, cyano, alkenyl, alkynyl, cycloalkyl, heterocycloalkyl, —(CH₂)ₙ₁Rₐ, —(CH₂)ₙ₁ORₐ, and —(CH₂)ₙ₁NRₐR_{b}, wherein the alkyl, deuterated alkyl, haloalkyl, alkoxy, haloalkoxy, amino, carbamoyl, sulfydryl, hydroxy, alkenyl, alkynyl, cycloalkyl, or heterocycloalkyl is optionally further substituted with one or more substituents selected from deuterium, substituted or unsubstituted cycloalkylalkyl, substituted or unsubstituted cycloalkylhaloalkyl, halogen, substituted or unsubstituted cycloalkylamino, sulfydryl, oxo, nitro, cyano, hydroxy, substituted or unsubstituted alkenyl, substituted or unsubstituted alkynyl, substituted or unsubstituted cycloalkylalkoxy, substituted or unsubstituted cycloalkylhaloalkoxy, substituted or unsubstituted cycloalkylhydroxyalkyl, substituted or unsubstituted cycloalkyl, substituted or unsubstituted heterocycloalkyl, —(CH₂)ₙ₁R_{c}, —(CH₂)ₙ₁OR_{c}, and —(CH₂)ₙ₁NR_{c}R_{d};
or any two adjacent or non-adjacent R₂ form one cycloalkyl or heterocycloalkyl group, wherein the cycloalkyl or heterocycloalkyl group is optionally further substituted with one or more substituents selected from deuterium, substituted or unsubstituted alkyl, substituted or unsubstituted haloalkyl, halogen, substituted or unsubstituted amino, oxo, nitro, cyano, hydroxy, substituted or unsubstituted alkenyl, substituted or unsubstituted alkynyl, substituted or unsubstituted alkoxy, substituted or unsubstituted haloalkoxy, substituted or unsubstituted hydroxyalkyl, substituted or unsubstituted cycloalkyl, and substituted or unsubstituted heterocycloalkyl;
Rₐ, R_{b}, R_{c}, and R_{d} are each independently selected from hydrogen, deuterium, alkyl, deuterated alkyl, haloalkyl, alkoxy, hydroxyalkyl, haloalkoxy, halogen, cyano, nitro, hydroxy, amino, carbamoyl, alkenyl, alkynyl, cycloalkyl, and heterocycloalkyl, wherein the alkyl, deuterated alkyl, haloalkyl, alkoxy, hydroxyalkyl, haloalkoxy, alkenyl, alkynyl, cycloalkyl, or heterocycloalkyl is optionally further substituted with one or more substituents selected from deuterium, substituted or unsubstituted alkyl, halogen, hydroxy, substituted or unsubstituted amino, oxo, nitro, cyano, substituted or unsubstituted alkenyl, substituted or unsubstituted alkynyl, substituted or unsubstituted alkoxy, substituted or unsubstituted hydroxyalkyl, substituted or unsubstituted cycloalkyl, and substituted or unsubstituted heterocycloalkyl;
R₃ is H, halogen, (C1-C3) alkyl, or cyclopropyl;
R₄ is 4- to 6-membered heterocycloalkyl containing 1 to 2 heteroatoms independently selected from N, S, and O, phenyl, or 5- to 6-membered heteroaryl containing 1 to 4 heteroatoms independently selected from N, S, and O, wherein the 4- to 6-membered heterocycloalkyl, phenyl, or 5- to 6-membered heteroaryl is optionally further substituted with one or more substituents selected from deuterium, alkyl, deuterated alkyl, haloalkyl, alkoxy, haloalkoxy, halogen, amino, nitro, hydroxy, cyano, cycloalkyl, heterocycloalkyl, oxoheterocycloalkyl, thioheterocycloalkyl, oxo, and thio; and
n is 0, 1, 2, or 3;
m is 0, 1, 2, 3, or 4;
n1 is 0, 1, 2, or 3.

In another preferred embodiment, the general formula (1) has a structure as shown in general formula (2):

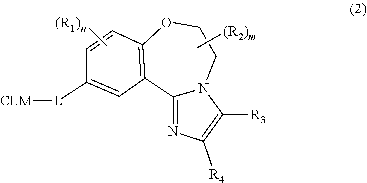

wherein CLM, L, R₁, R₂, R₃, R₄, m, and n are as defined above.

In another preferred embodiment, in the general formula (1) or general formula (2), CLM is a group that can covalently bind to a cysteine residue (Cys) in the PI3Kα protein and comprises a carbon-carbon double bond or carbon-carbon triple bond.

In another referred embodiment in the general formula 1 or general formula 2 CLM is —NHCN, —CN,

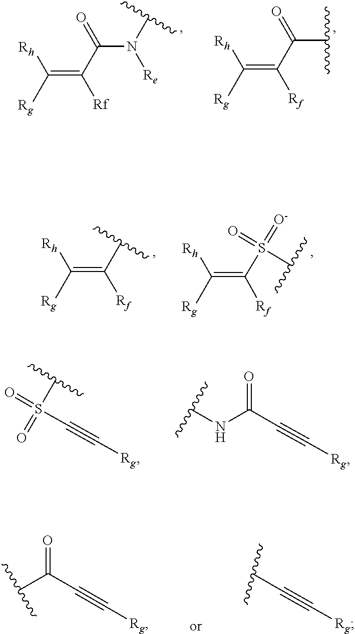

Rₑ is H, substituted or unsubstituted (C1-C6) alkyl, or substituted or unsubstituted (C3-C6) cycloalkyl;
R_{f}, R_{g}, and Rₕ are each independently H, halogen, —CN, —C(O)R, —C(O)OR, —C(O)NR₂, —C(O)N(R)OR, —(CH₂)_{w}R, —(CH₂)_{w}OR, —(CH₂)_{w}N(R)₂, substituted or unsubstituted (C1-C6) alkyl, substituted or unsubstituted (C3-C6) cycloalkyl, substituted or unsubstituted phenyl, substituted or unsubstituted 3- to 7-membered saturated or partially unsaturated heterocycloalkyl containing 1 to 2 heteroatoms independently selected from N, S, and O, or substituted or unsubstituted 5- to 6-membered heteroaryl containing 1 to 4 heteroatoms independently selected from N, S, and O;
or Rₑ and R_{f}, R_{f} and R_{g}, R_{g} and Rₕ, or Rₑ and Rₕ may form a substituted or unsubstituted 4- to 7-membered saturated or partially unsaturated ring containing 0 to 2 heteroatoms independently selected from N, S, and O, wherein two hydrogen atoms on the same carbon atom of the 4- to 7-membered saturated or partially unsaturated ring may be substituted with oxygen to form oxo;
each R is independently H, substituted or unsubstituted (C1-C6) alkyl, substituted or unsubstituted (C3-C6) cycloalkyl, substituted or unsubstituted phenyl, substituted or unsubstituted 3- to 7-membered saturated or partially unsaturated heterocycloalkyl containing 1 to 2 heteroatoms independently selected from N, S, and O, or substituted or unsubstituted 5- to 6-membered heteroaryl containing 1 to 4 heteroatoms independently selected from N, S, and O;
w is 0, 1, or 2.

In another referred embodiment, in the general formula (1) or general formula (2), CLM is —NHCN, —CN,

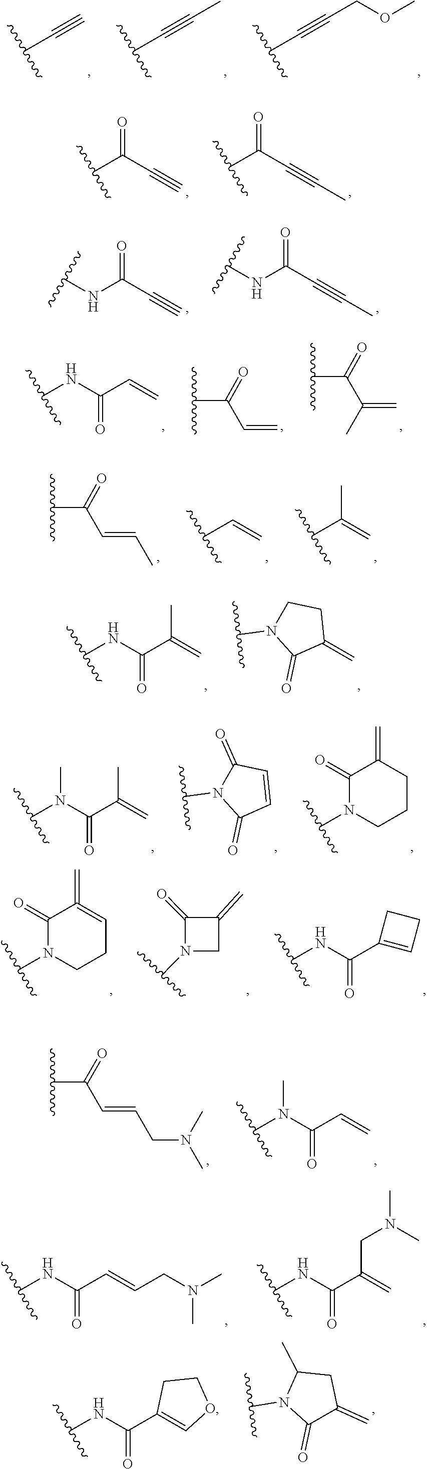

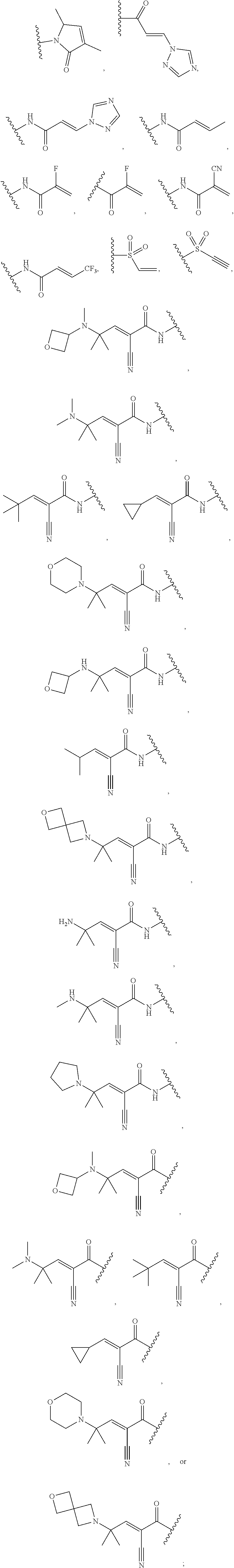

CLM is preferably —NHCN, —CN,

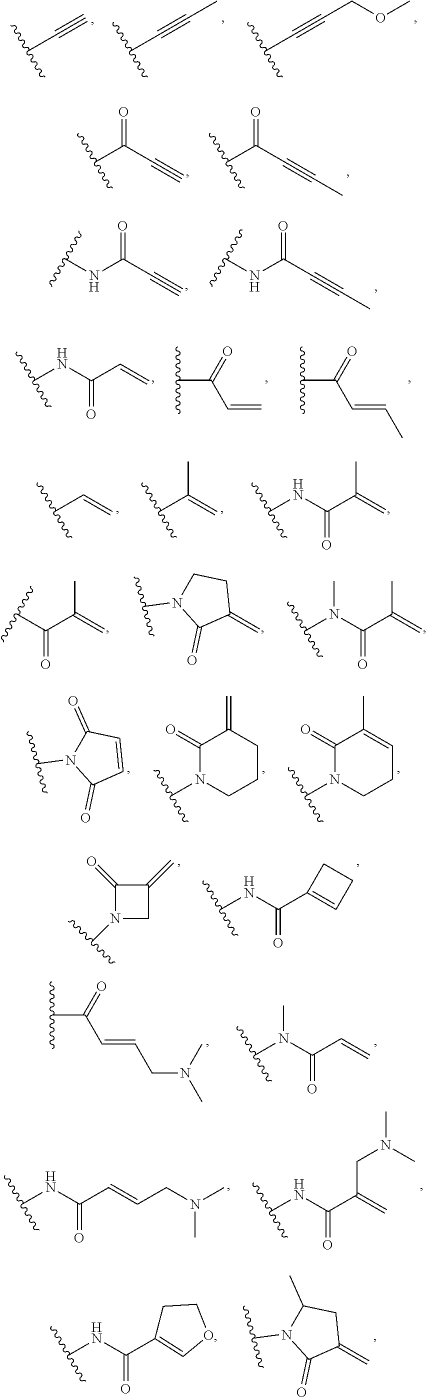

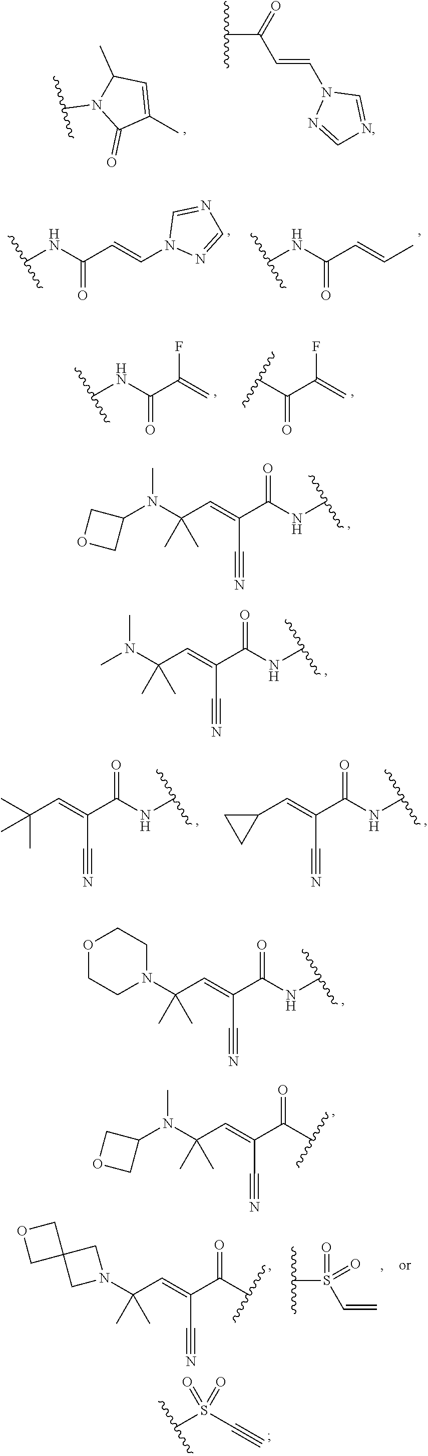

CLM is more preferably

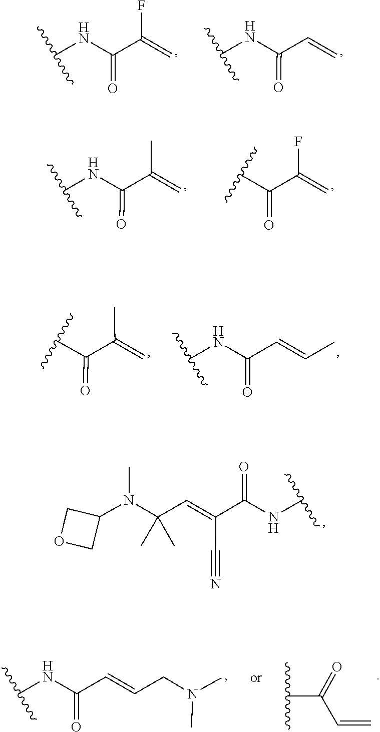

In another preferred embodiment, in the general formula (1) or general formula (2), L is

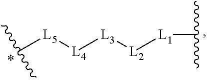

wherein L₁, L₂, L₃, L₄, and L₅ are each independently selected from a chemical bond, O, S, NH, C(═O), C(═O)NH, S(═O), S(═O)₂, (C1-C6) alkylene, —(C1-C6) alkylene-O—, (C2-C3) alkenylene, (C2-C3) alkynylene, (C3-C10) cycloalkylene, phenylene, 5- to 11-membered bridged cyclylene, 3- to 10-membered saturated or partially unsaturated heterocycloalkylene containing 1 to 4 heteroatoms independently selected from N, S, and O, 7- to 11-membered spiro heterocyclylene, 5- to 11-membered bridged heterocyclylene, and 5- to 9-membered heteroarylene, wherein the (C1-C6) alkylene, —(C1-C6) alkylene-O—, (C2-C3) alkenylene, (C2-C3) alkynylene, (C3-C10) cycloalkylene, 3- to 10-membered saturated or partially unsaturated heterocycloalkylene, phenylene, 7- to 11-membered spiro heterocyclylene, 5- to 11-membered bridged heterocyclylene, or 5- to 9-membered heteroarylene is optionally substituted with 1, 2, or 3 R_{L};
each R_{L} is independently selected from H, halogen, OH, NH₂, CN, —CONH₂, (C1-C6) alkyl, (C3-C6) cycloalkyl, (C1-C6) alkyl-C(═O)—, (C1-C6) alkoxy, (C1-C6) alkylthio, and (C1-C6) alkylamino, wherein the (C1-C6) alkyl, (C3-C6) cycloalkyl, (C1-C6) alkyl-C(═O)—, (C1-C6) alkoxy, (C1-C6) alkylthio, or (C1-C6) alkylamino is optionally substituted with 1, 2, or 3 R_{LL};
each R_{LL} is independently selected from H, halogen, (C1-C6) alkyl, OH, NH₂, MeNH—, Me₂N—, CH₃, CH₂F, CHF₂, and CF₃; wherein * denotes the connection to CLM.

In another preferred embodiment, in the general formula (1) or general formula (2), each of L₁, L₂, L₃, L₄, and L₅ is independently a chemical bond, O, S, NH, C(═O), C(═O)NH, S(═O), S(═O)₂, CH₂,

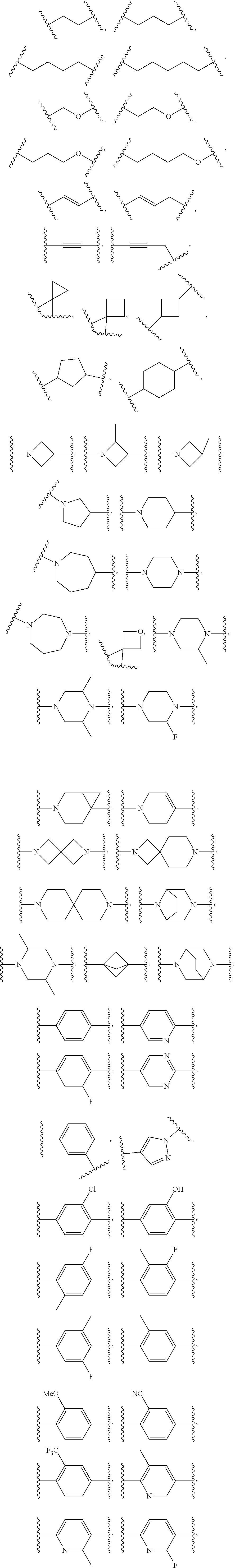

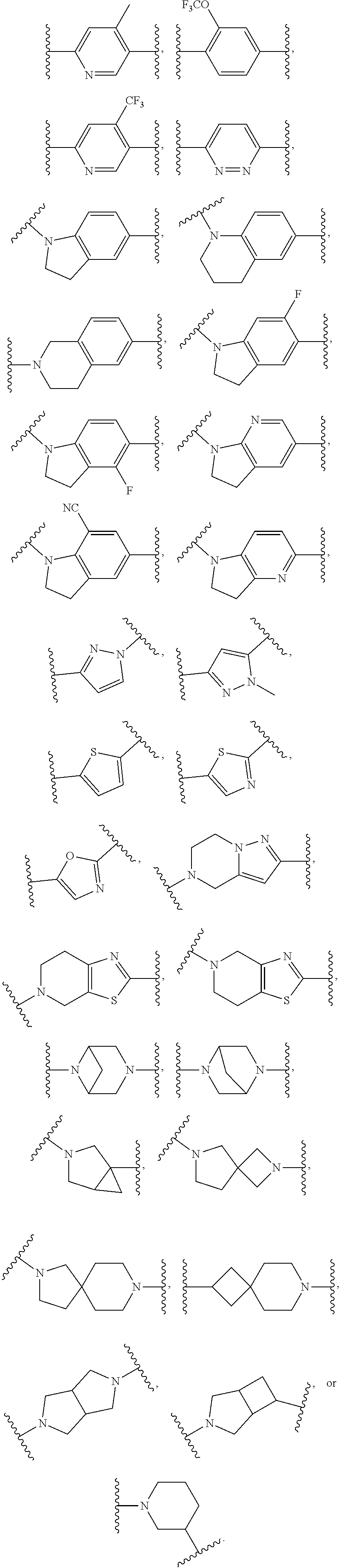

In another preferred embodiment, in the general formula (1) or general formula (2),
L₁ is a chemical bond, (C2-C3) alkenylene, (C2-C3) alkynylene, (C3-C10) cycloalkylene, 3- to 10-membered saturated or partially unsaturated heterocycloalkylene containing 1 to 4 heteroatoms independently selected from N, S, and O, 7- to 11-membered spiro heterocyclylene, or 5- to 11-membered bridged heterocyclylene, wherein the (C2-C3) alkenylene, (C2-C3) alkynylene, (C3-C10) cycloalkylene, 3- to 10-membered saturated or partially unsaturated heterocycloalkylene, 7- to 11-membered spiro heterocyclylene, or 5- to 11-membered bridged heterocyclylene is optionally substituted with 1, 2, or 3 R_{L};
L₂ is a chemical bond, C(═O), C(═O)NH, (C1-C6) alkylene, or —(C1-C6) alkylene-O—;
L₃ is a chemical bond, 3- to 10-membered saturated or partially unsaturated heterocycloalkylene containing 1 to 4 heteroatoms independently selected from N, S, and O, 7- to 11-membered spiro heterocyclylene, or 5- to 11-membered bridged heterocyclylene, wherein the 3- to 10-membered saturated or partially unsaturated heterocycloalkylene, 7- to 11-membered spiroheterocyclylene, or 5- to 11-membered bridged heterocyclylene is optionally substituted with 1, 2, or 3 R_{L};
L₄ is a chemical bond, C(═O), C(═O)NH, (C1-C6) alkylene, or —(C1-C6) alkylene-O—;
L₅ is phenylene, 5- to 9-membered heteroarylene, 3- to 10-membered saturated or partially unsaturated heterocycloalkylene containing 1 to 4 heteroatoms independently selected from N, S, and O, 7- to 11-membered spiro heterocyclylene, or 5- to 11-membered bridged heterocyclylene, wherein the phenylene, 5- to 9-membered heteroarylene, 3- to 10-membered saturated or partially unsaturated heterocycloalkylene, 7- to 11-membered spiro heterocyclylene, or 5- to 11-membered bridged heterocyclylene is optionally substituted with 1, 2, or 3 R_{L}.

In another preferred embodiment, in the general formula (1) or general formula (2),
L₁ is 3- to 10-membered saturated or partially unsaturated heterocycloalkylene containing 1 to 4 heteroatoms independently selected from N, S, and O, 7- to 11-membered spiro heterocyclylene, or 5- to 11-membered bridged heterocyclylene, wherein the 3- to 10-membered saturated or partially unsaturated heterocycloalkylene, 7- to 11-membered spiro heterocyclylene, or 5- to 11-membered bridged heterocyclylene is optionally substituted with 1, 2, or 3 R_{L};
L₂ is a chemical bond;
L₃ is 3- to 10-membered saturated or partially unsaturated heterocycloalkylene containing 1 to 4 heteroatoms independently selected from N, S, and O, 7- to 11-membered spiro heterocyclylene, or 5- to 11-membered bridged heterocyclylene, wherein the 3- to 10-membered saturated or partially unsaturated heterocycloalkylene, 7- to 11-membered spiro heterocyclylene, or 5- to 11-membered bridged heterocyclylene is optionally substituted with 1, 2, or 3 R_{L};
L₄ is a chemical bond;
L₅ is phenylene, 5- to 9-membered heteroarylene, or 3- to 10-membered saturated or partially unsaturated heterocycloalkylene containing 1 to 4 heteroatoms independently selected from N, S, and O, wherein the phenylene, 5- to 9-membered heteroarylene, or 3- to 10-membered saturated or partially unsaturated heterocycloalkylene is optionally substituted with 1, 2, or 3 R_{L}.

In another preferred embodiment, in the general formula (1) or general formula (2), the structural unit

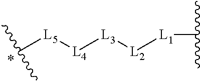

is selected from

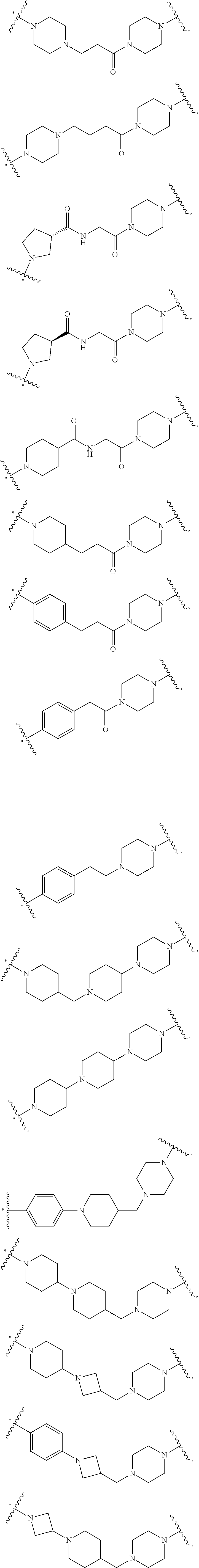

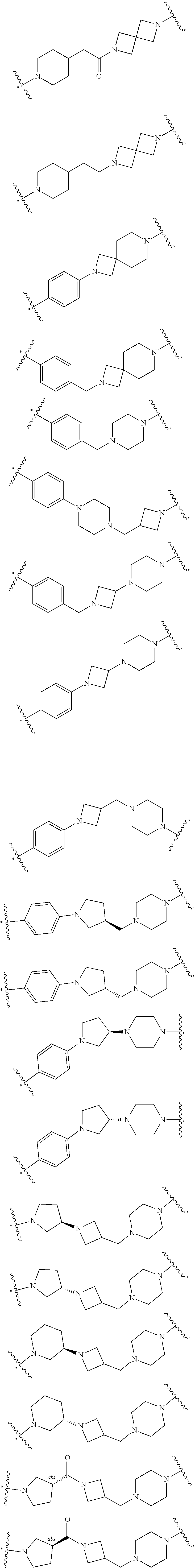

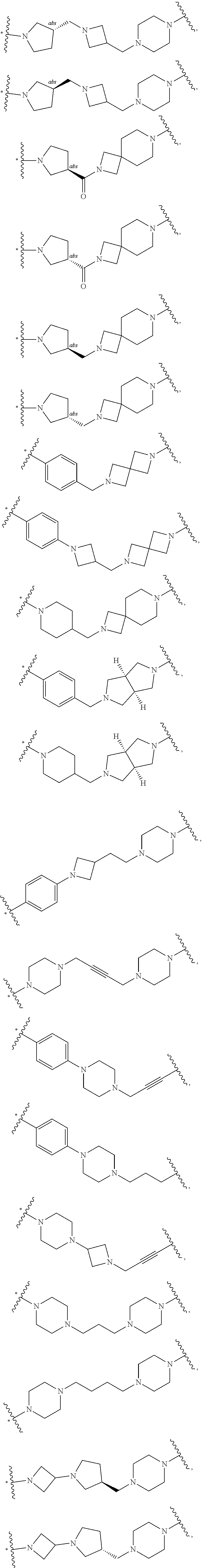

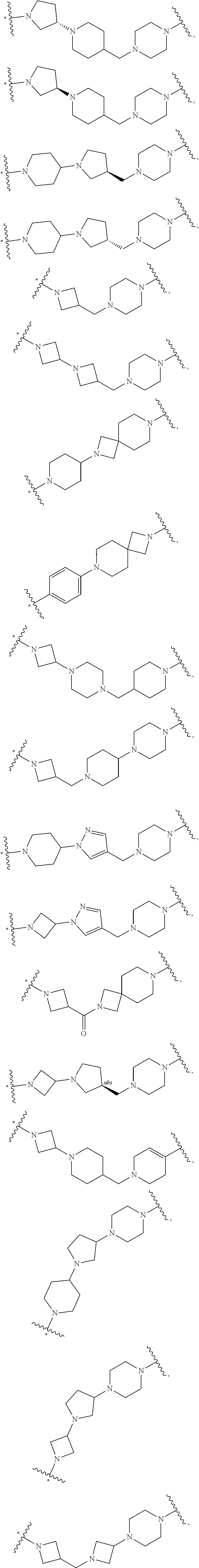

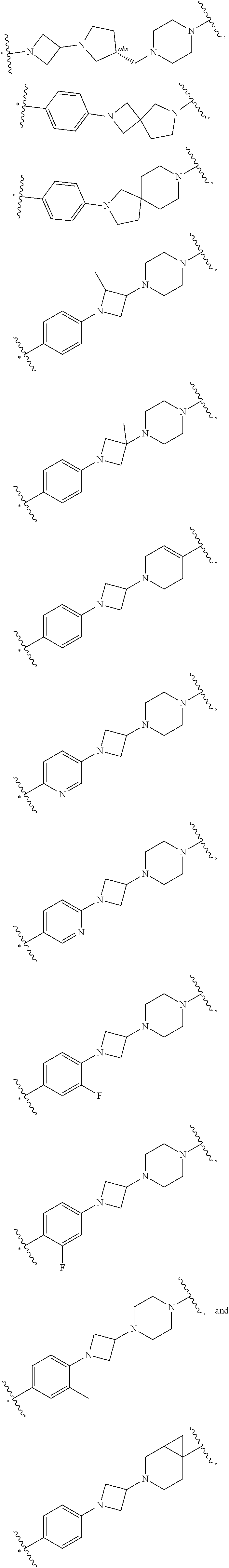

wherein * denotes the connection to CLM.

In another preferred embodiment, in the general formula (1) or general formula (2), each of R₁ and R₂ is independently selected from H, deuterium, alkyl, deuterated alkyl, haloalkyl, alkoxy, hydroxy, haloalkoxy, halogen, amino, carbamoyl, —(CH₂)ₙ₁Rₐ, —(CH₂)ₙ₁ORₐ, and —(CH₂)ₙ₁NRₐR_{b}, wherein the alkyl, deuterated alkyl, haloalkyl, alkoxy, haloalkoxy, amino, and carbamoyl are optionally further substituted with one or more substituents selected from deuterium, substituted or unsubstituted cycloalkylalkyl, substituted or unsubstituted cycloalkylhaloalkyl, halogen, substituted or unsubstituted cycloalkylamino, sulfydryl, oxo, cyano, hydroxy, substituted or unsubstituted cycloalkyl, substituted or unsubstituted heterocyclyl, —(CH₂)ₙ₁R_{c}, —(CH₂)ₙ₁OR_{c}, and —(CH₂)ₙ₁NR_{c}R_{d};
or any two adjacent or non-adjacent R₂ are linked to form one cycloalkyl or heterocycloalkyl group, wherein the cycloalkyl and heterocycloalkyl groups are optionally further substituted with one or more substituents selected from deuterium, substituted or unsubstituted alkyl, substituted or unsubstituted haloalkyl, halogen, substituted or unsubstituted amino, oxo, and hydroxy;
Rₐ, R_{b}, R_{c}, and R_{d} are each independently selected from hydrogen, deuterium, alkyl, deuterated alkyl, haloalkyl, alkoxy, hydroxyalkyl, haloalkoxy, halogen, hydroxy, amino, carbamoyl, cycloalkyl, and heterocycloalkyl, wherein the alkyl, deuterated alkyl, haloalkyl, alkoxy, hydroxyalkyl, haloalkoxy, cycloalkyl, and heterocycloalkyl are optionally further substituted with one or more substituents selected from deuterium, substituted or unsubstituted alkyl, halogen, hydroxy, substituted or unsubstituted amino, oxo, substituted or unsubstituted alkoxy, substituted or unsubstituted hydroxyalkyl, substituted or unsubstituted cycloalkyl, and substituted or unsubstituted heterocycloalkyl;
R₃ is H, halogen, cyclopropyl, or CH₃;
R₄ is 4- to 6-membered saturated heterocycloalkyl containing 1 to 2 heteroatoms independently selected from N, S, and O, phenyl, or 5- to 6-membered heteroaryl containing 1 to 4 heteroatoms independently selected from N, S, and O, wherein the 4- to 6-membered saturated heterocycloalkyl, phenyl, or 5- to 6-membered heteroaryl is optionally further substituted with one or more substituents selected from deuterium, alkyl, deuterated alkyl, haloalkyl, alkoxy, haloalkoxy, halogen, amino, nitro, hydroxy, cyano, cycloalkyl, heterocyclyl, oxoheterocycloalkyl, thioheterocycloalkyl, oxo, and thio; and
n is 0, 1, or 2;
m is 0, 1, or 2;
n1 is 0 or 1.

In another preferred embodiment, in the general formula (1) or general formula (2), each of R₁ and R₂ is independently selected from H, deuterium, methyl, deuterated methyl, haloalkyl, methoxy, hydroxy, halomethoxy, F, Cl, amino, carbamoyl,

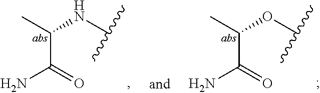

or when m is 2, two R₂ attached to the same carbon atom may form cyclopropyl.

In another preferred embodiment, in the general formula (1) or general formula (2), R₃ is H, F, Cl, cyclopropyl, or CH₃.

In another preferred embodiment, the general formula (1) or general formula (2) has a structure as shown in general formula (3):

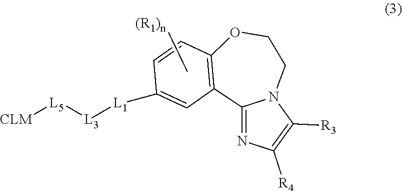

wherein
L₁ is 3- to 10-membered saturated or partially unsaturated heterocycloalkylene containing 1 to 4 heteroatoms independently selected from N, S, and O, 7- to 11-membered spiro heterocyclylene, or 5- to 11-membered bridged heterocyclylene, wherein the 3- to 10-membered saturated or partially unsaturated heterocycloalkylene, 7- to 11-membered spiro heterocyclylene, or 5- to 11-membered bridged heterocyclylene is optionally substituted with 1, 2, or 3 R_{L};
L₃ is 3- to 10-membered saturated or partially unsaturated heterocycloalkylene containing 1 to 4 heteroatoms independently selected from N, S, and O, 7- to 11-membered spiro heterocyclylene, or 5- to 11-membered bridged heterocyclylene, wherein the 3- to 10-membered saturated or partially unsaturated heterocycloalkylene, 7- to 11-membered spiro heterocyclylene, or 5- to 11-membered bridged heterocyclylene is optionally substituted with 1, 2, or 3 R_{L};
L₅ is phenylene, 5- to 9-membered heteroarylene, or 3- to 10-membered saturated or partially unsaturated heterocycloalkylene containing 1 to 4 heteroatoms independently selected from N, S, and O, wherein the phenylene, 5- to 9-membered heteroarylene, or 3- to 10-membered saturated or partially unsaturated heterocycloalkylene is optionally substituted with 1, 2, or 3 R_{L};
each R_{L} is independently selected from H, F, Cl, hydroxy, amino, cyano, amido, methyl, methoxy, trifluoromethyl, difluoromethyl, monofluoromethyl, trifluoromethoxy, methylamino, and dimethylamino;
CLM is

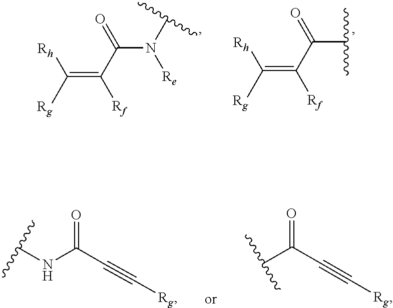

R₁ is independently selected from hydrogen, deuterium, (C1-C3) alkyl, (C1-C3) deuterated alkyl, (C1-C3) haloalkyl, (C1-C3) alkoxy, (C1-C3) haloalkoxy, halogen, amino, methylamino, hydroxy, cyano, (C3-C6) cycloalkyl, and (3- to 6-membered) heterocycloalkyl; n is 0, 1, or 2;
R₃ is H, halogen, (C1-C3) alkyl, or cyclopropyl;
R₄ is 4- to 6-membered heterocycloalkyl containing 1 to 2 heteroatoms independently selected from N, S, and O, phenyl, or 5- to 6-membered heteroaryl containing 1 to 4 heteroatoms independently selected from N, S, and O, wherein the 4- to 6-membered heterocycloalkyl, phenyl, or 5- to 6-membered heteroaryl is optionally further substituted with one or more substituents selected from deuterium, (C1-C3) alkyl, (C1-C3) deuterated alkyl, (C1-C3) haloalkyl, (C1-C3) alkoxy, (C1-C3) haloalkoxy, halogen, amino, nitro, hydroxy, cyano, (C3-C6) cycloalkyl, (3- to 6-membered) heterocycloalkyl, (3- to 6-membered) oxoheterocycloalkyl, (3- to 6-membered) thioheterocycloalkyl, oxo, and thio.

In another preferred embodiment, in the general formula (3),
L₁ is 4- to 10-membered saturated or partially unsaturated heterocycloalkylene containing 1 to 4 heteroatoms independently selected from N, S, and O or 5- to 11-membered bridged heterocyclylene, wherein the 4- to 10-membered saturated or partially unsaturated heterocycloalkylene or 5- to 11-membered bridged heterocyclylene is optionally substituted with 1, 2, or 3 R_{L}; L₁ is preferably

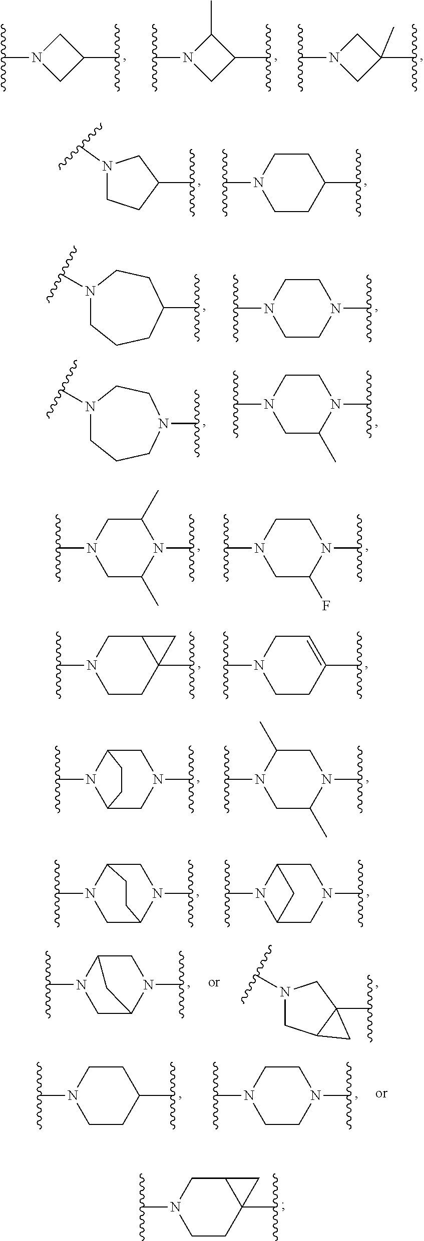

more preferably
L₃ is 4- to 10-membered saturated or partially unsaturated heterocycloalkylene containing 1 to 4 heteroatoms independently selected from N, S, and O or 5- to 11-membered bridged heterocyclylene, wherein the 4- to 10-membered saturated or partially unsaturated heterocycloalkylene or 5- to 11-membered bridged heterocyclene is optionally substituted with 1, 2, or 3 R_{L}; L₃ is preferably

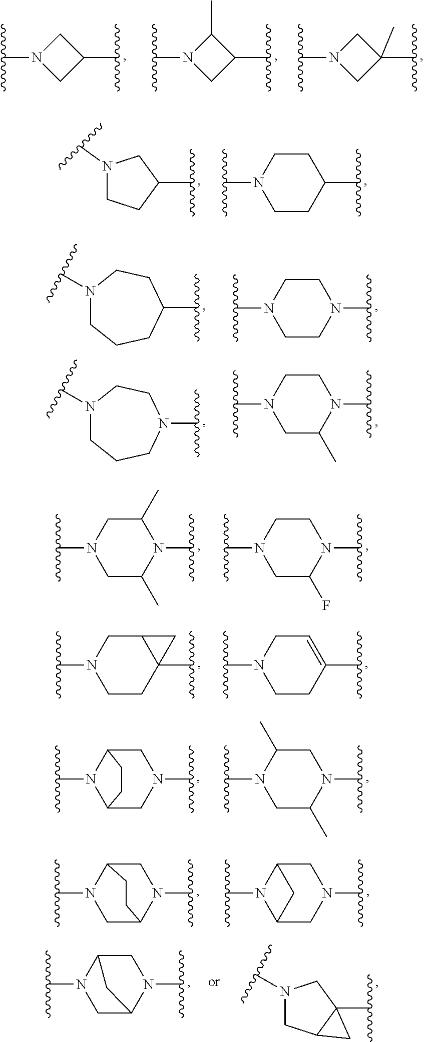

more preferably

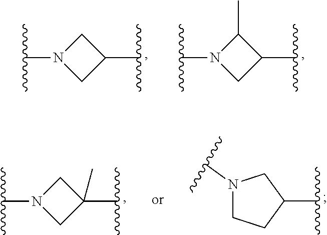

L₅ is phenylene, 5- to 6-membered heteroarylene, or 4- to 10-membered saturated or partially unsaturated heterocycloalkylene containing 1 to 4 heteroatoms independently selected from N, S, and O, wherein the phenylene, 5- to 6-membered heteroarylene, or 4- to 10-membered saturated or partially unsaturated heterocycloalkylene is optionally substituted with 1, 2, or 3 R_{L}; L₅ is preferably

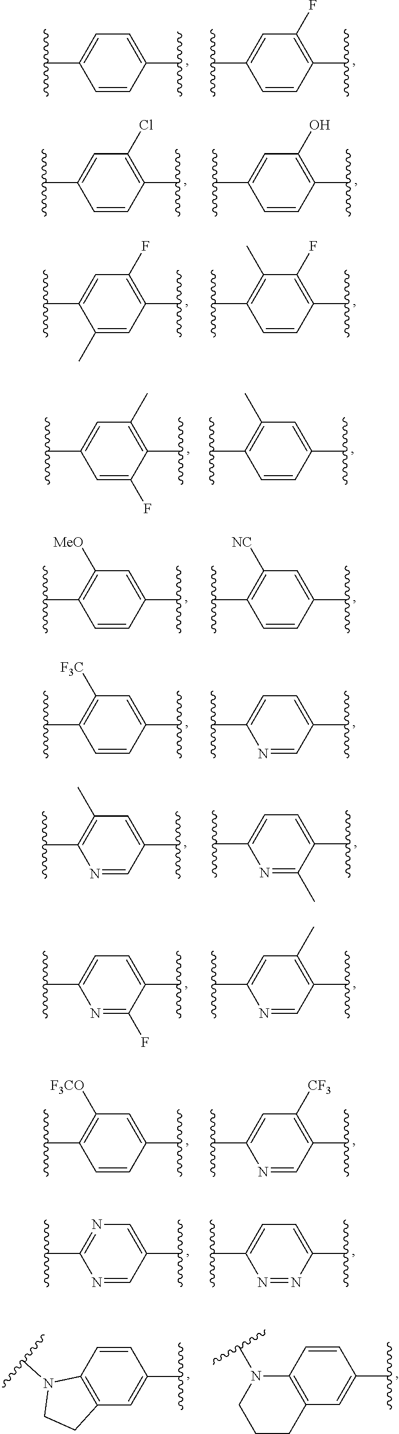

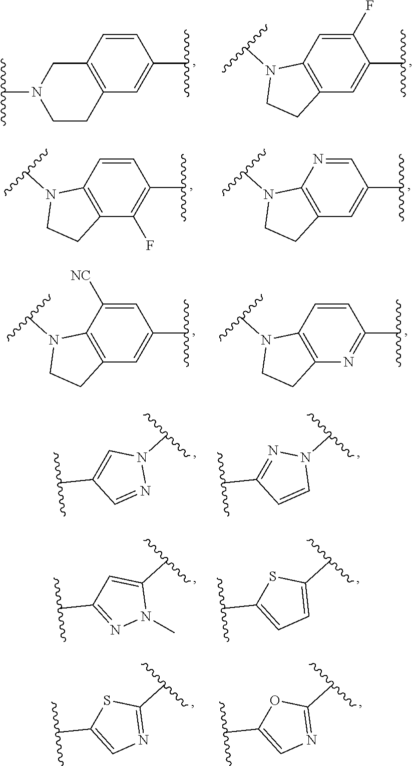

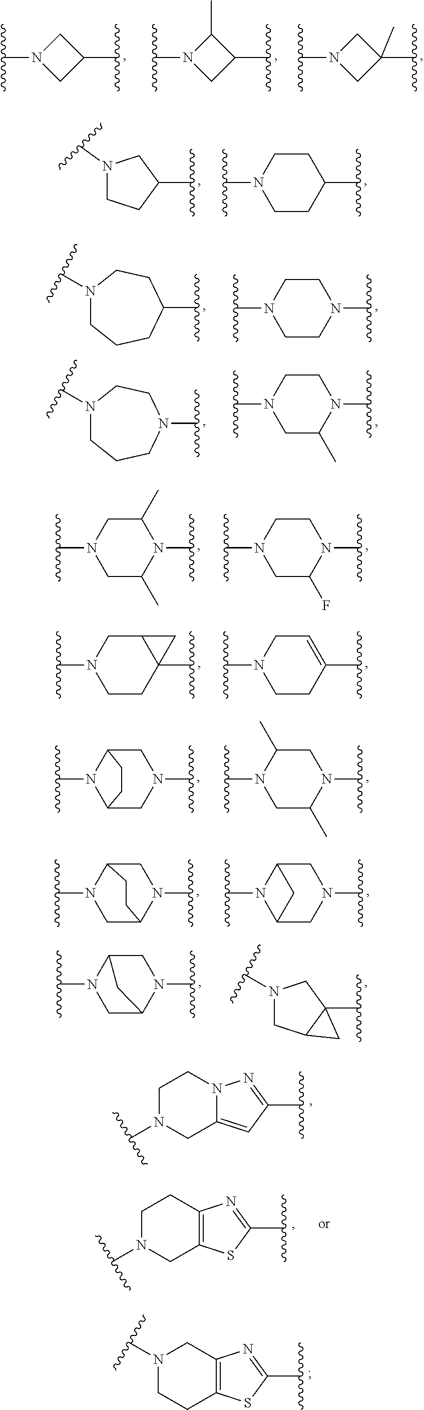

### CLM is

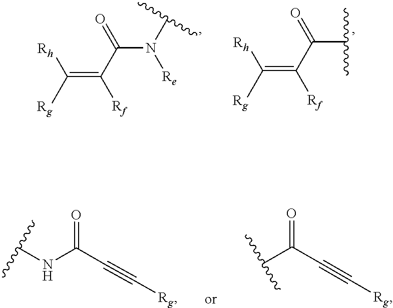

Rₑ is H or Me;
R_{f} is H, F, Me, or CN;
R_{g} and Rₕ are each independently H, halogen, —CN, —(CH₂)_{w}R, —(CH₂)_{w}OR, —(CH₂)_{w}N(R)₂, substituted or unsubstituted (C1-C6) alkyl, substituted or unsubstituted (C3-C6) cycloalkyl, or substituted or unsubstituted 3- to 7-membered saturated or partially unsaturated heterocycloalkyl containing 1 to 2 heteroatoms independently selected from N, S, and O, wherein the substituted or unsubstituted (C1-C6) alkyl is preferably

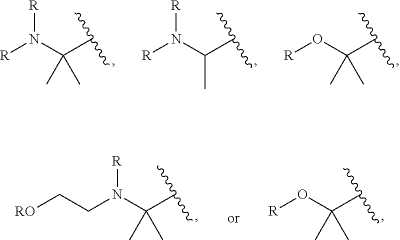

and the substituted or unsubstituted (C3-C6) cycloalkyl is preferably

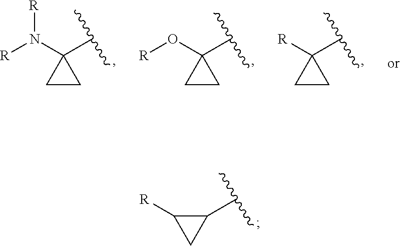

each R is independently H, (C1-C6) alkyl, (C3-C6) cycloalkyl, 3- to 7-membered saturated or partially unsaturated heterocycloalkyl containing 1 to 2 heteroatoms independently selected from N, S, and O, 7- to 11-membered spiro heterocycloalkyl, or 5- to 11-membered bridged heterocycloalkyl, wherein the (C1-C6) alkyl, (C3-C6) cycloalkyl, 3- to 7-membered saturated or partially unsaturated heterocycloalkyl containing 1 to 2 heteroatoms independently selected from N, S, and O, 7- to 11-membered spiro heterocycloalkyl, or 5- to 11-membered bridged heterocycloalkyl is optionally further substituted with one or more substituents selected from deuterium, (C1-C6) alkyl, (C1-C6) alkoxy, (C3-C6) cycloalkyl, 3- to 7-membered saturated or partially unsaturated heterocycloalkyl containing 1 to 2 heteroatoms independently selected from N, S, and O, 7- to 11-membered spiro heterocyclyl, 5- to 11-membered bridged heterocyclyl, oxo, —C(O)Me, —C(O)OMe, —C(O)OBu-t, —F, C1, —OH, and —NH₂;
w is 0, 1, or 2;
R₁ is independently selected from hydrogen, deuterium, -Me, —OMe, —OCD₃, —CD₃, —CHF₂, —CF₃, F, Cl, —NH₂, —NHMe, hydroxy, cyano, and cyclopropyl; n is 0, 1, or 2;
R₃ is H, F, Cl, -Me, or cyclopropyl;
R₄ is 5-membered heterocycloalkyl containing 1 to 2 heteroatoms independently selected from N, S, and O or 5-membered heteroaryl containing 1 to 3 heteroatoms independently selected from N, S, and O, wherein the 5-membered heterocycloalkyl or 5-membered heteroaryl is optionally further substituted with one or more substituents selected from deuterium, (C1-C3) alkyl, (C1-C3) deuterated alkyl, (C1-C3) haloalkyl, (C1-C3) alkoxy, (C1-C3) haloalkoxy, halogen, amino, nitro, hydroxy, cyano, (C3-C6) cycloalkyl, (3- to 6-membered) heterocycloalkyl, (3- to 6-membered) oxoheterocycloalkyl, (3- to 6-membered) thioheterocycloalkyl, oxo, and thio.

In another preferred embodiment, in the general formula (3),
L₁ is 4- to 10-membered saturated or partially unsaturated heterocycloalkylene containing 1 to 4 heteroatoms independently selected from N, S, and O or 5- to 11-membered bridged heterocyclylene, wherein the 4- to 10-membered saturated or partially unsaturated heterocycloalkylene or 5- to 11-membered bridged heterocyclylene is optionally substituted with 1, 2, or 3 R_{L}; L₁ is preferably 4- to 7-membered saturated or partially unsaturated heterocycloalkylene containing 1 to 2 N atoms or 6- to 9-membered bridged heterocyclylene containing 1 to 2 N atoms, wherein the 4- to 7-membered saturated or partially unsaturated heterocycloalkylene or 6- to 9-membered bridged heterocyclylene is optionally substituted with 1 to 2 R_{L}; L₁ is preferably

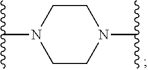 [SMILES: *N1CCN(*)CC1]

L₃ is 4- to 10-membered saturated or partially unsaturated heterocycloalkylene containing 1 to 4 heteroatoms independently selected from N, S, and O or 5- to 11-membered bridged heterocyclylene, wherein the 4- to 10-membered saturated or partially unsaturated heterocycloalkylene or 5- to 11-membered bridged heterocyclylene is optionally substituted with 1, 2, or 3 R_{L}; L₃ is preferably 4- to 7-membered saturated or partially unsaturated heterocycloalkylene containing 1 to 2 N atoms or 6- to 9-membered bridged heterocyclylene containing 1 to 2 N atoms, wherein the 4- to 7-membered saturated or partially unsaturated heterocycloalkylene or 6- to 9-membered bridged heterocyclylene is optionally substituted with 1 to 2 R_{L}; L₃ is preferably

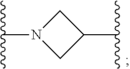 [SMILES: *C1CN(*)C1]

L₅ is phenylene, 5- to 6-membered heteroarylene containing 1 to 2 heteroatoms independently selected from N, S, and O, or 4- to 10-membered saturated or partially unsaturated heterocycloalkylene containing 1 to 4 heteroatoms independently selected from N, S, and O, wherein the phenylene, 5- to 6-membered heteroarylene, or 4- to 10-membered saturated or partially unsaturated heterocycloalkylene is optionally substituted with 1, 2, or 3 R_{L}; L₅ is preferably phenylene, 5- to 6-membered heteroarylene containing 1 to 2 heteroatoms independently selected from N, S, and O, or 8- to 10-membered partially unsaturated bicyclic heterocycloalkylene containing 1 to 3 heteroatoms independently selected from N, S, and O, wherein the phenylene, 5- to 6-membered heteroarylene, or 8- to 10-membered partially unsaturated bicyclic heterocycloalkylene is optionally substituted with 1 to 2 R_{L}; L₅ is preferably

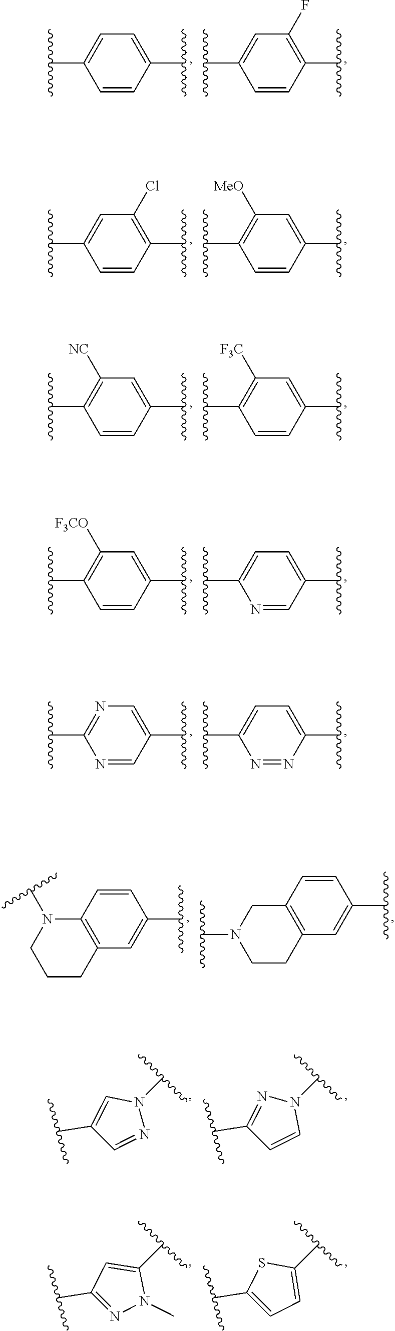

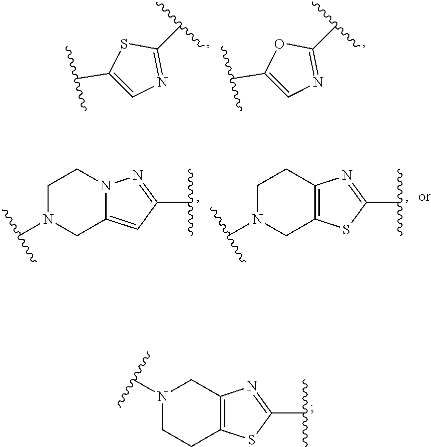 [SMILES: *c1cnc(*)s1 ; *c1cnc(*)o1 ; *c1cc2n(n1)CCN(*)C2 ; *c1nc2c(s1)CN(*)CC2 ; *c1nc2c(s1)CCN(*)C2]

each R_{L} is independently selected from H, F, Cl, hydroxy, amino, cyano, amido, methyl, methoxy, trifluoromethyl, difluoromethyl, monofluoromethyl, trifluoromethoxy, methylamino, and dimethylamino;
CLM is

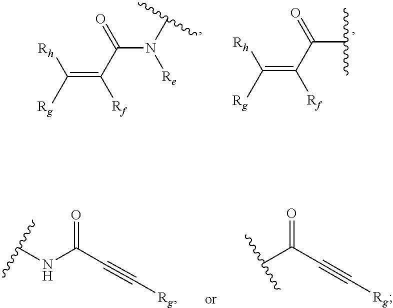

Rₑ is H or Me;
R_{f} is H, F, Me, or CN;
R_{g} and Rₕ are each independently H, halogen, —CN, —(CH₂)_{w}R, —(CH₂)_{w}OR, —(CH₂)_{w}N(R)₂, substituted or unsubstituted (C1-C6) alkyl, substituted or unsubstituted (C3-C6) cycloalkyl, or substituted or unsubstituted 3- to 7-membered saturated or partially unsaturated heterocycloalkyl containing 1 to 2 heteroatoms independently selected from N, S, and O, wherein the substituted or unsubstituted (C1-C6) alkyl is preferably

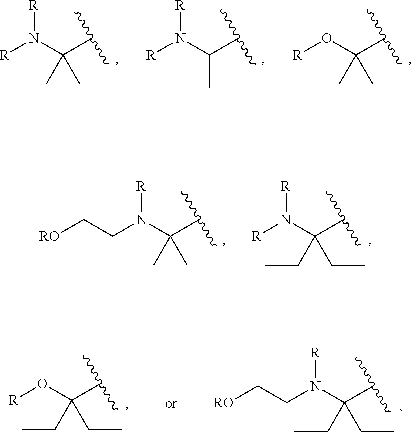

and the substituted or unsubstituted (C3-C6) cycloalkyl is preferably

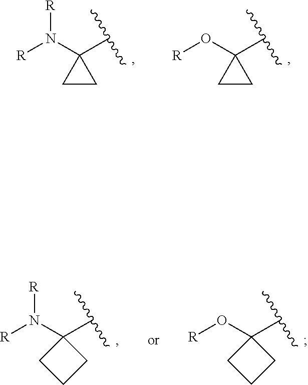

each R is independently H, —C(O)Rₘ, —C(O)ORₘ, —S(O)₂Rₘ, —CH₂OC(O)Rₘ,

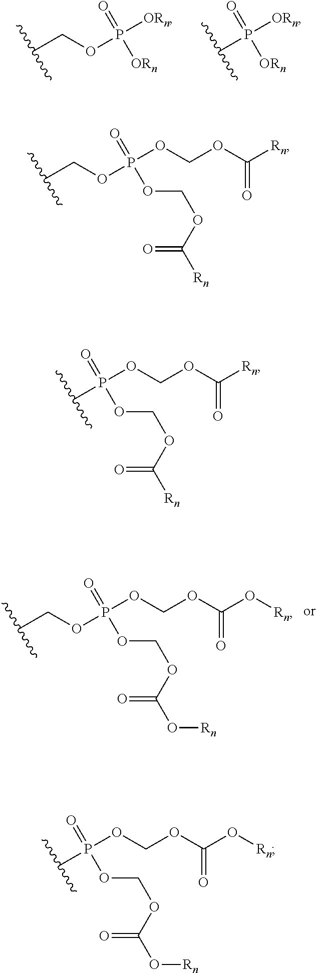

Rₘ and Rₙ are each independently H, (C1-C18) alkyl, (C3-C6) cycloalkyl, 3- to 7-membered saturated or partially unsaturated heterocycloalkyl containing 1 to 2 heteroatoms independently selected from N, S, and O, 7- to 11-membered spiro heterocycloalkyl, or 5- to 11-membered bridged heterocycloalkyl, wherein the (C1-C18) alkyl, (C3-C6) cycloalkyl, 3- to 7-membered saturated or partially unsaturated heterocycloalkyl containing 1 to 2 heteroatoms independently selected from N, S, and O, 7- to 11-membered spiro heterocycloalkyl, or 5- to 11-membered bridged heterocycloalkyl is optionally further substituted with one or more substituents selected from deuterium, (C1-C6) alkyl, (C1-C6) alkoxy, (C3-C6) cycloalkyl, 3- to 7-membered saturated or partially unsaturated heterocycloalkyl containing 1 to 2 heteroatoms independently selected from N, S, and O, 7- to 11-membered spiro heterocyclyl, 5- to 11-membered bridged heterocyclyl, oxo, —C(O)Me, —C(O)OMe, —C(O)OBu-t, —F, Cl, —OH, and —NH₂; Rₘ and Rₙ are each independently preferably H, methyl, ethyl, tert-butyl, isopropyl, cyclopropyl, or cyclobutyl;
w is 0, 1, or 2;
R₁ is independently selected from hydrogen, deuterium, -Me, —OMe, —OCD₃, —CD₃, —CHF₂, —CF₃, F, Cl, —NH₂, —NHMe, hydroxy, cyano, and cyclopropyl; n is 0, 1, or 2;
R₃ is H, F, Cl, -Me, or cyclopropyl;
R₄ is 5-membered heterocycloalkyl containing 1 to 2 heteroatoms independently selected from N, S, and O or 5-membered heteroaryl containing 1 to 3 heteroatoms independently selected from N, S, and O, wherein the 5-membered heterocycloalkyl or 5-membered heteroaryl is optionally further substituted with one or more substituents selected from deuterium, (C1-C3) alkyl, (C1-C3) deuterated alkyl, (C1-C3) haloalkyl, (C1-C3) alkoxy, (C1-C3) haloalkoxy, halogen, amino, nitro, hydroxy, cyano, (C3-C6) cycloalkyl, (3- to 6-membered) heterocycloalkyl, (3- to 6-membered) oxoheterocycloalkyl, (3- to 6-membered) thioheterocycloalkyl, oxo, and thio.

In another preferred embodiment, in the general formulas (1)-(3), R₄ is preferably 5-membered heterocycloalkyl containing 1 N atom, 5-membered heterocycloalkyl containing 1 N and 1 S or 0, or 5-membered heteroaryl containing 1, 2, or 3 heteroatoms independently selected from N, S, and O, wherein the 5-membered heterocycloalkyl or 5-membered heteroaryl is optionally further substituted with 1 or 2 substituents independently selected from -D, -Me, —CD₃, —CH₂F, —CHF₂, —CF₃, —OMe, —OCD₃, oxo, and thio.

In another preferred embodiment, in the general formulas (1)-(3), Rₐ is selected from

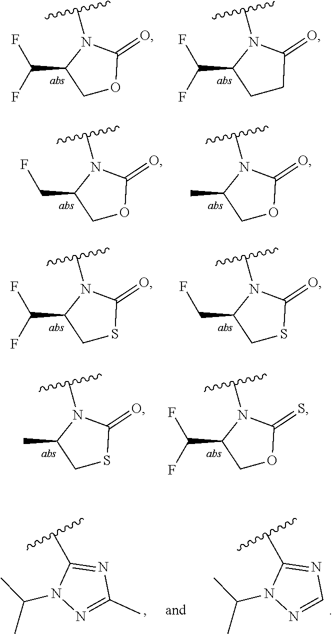

In another specific embodiment of the present disclosure, the compound of the present disclosure has one of the following structures:

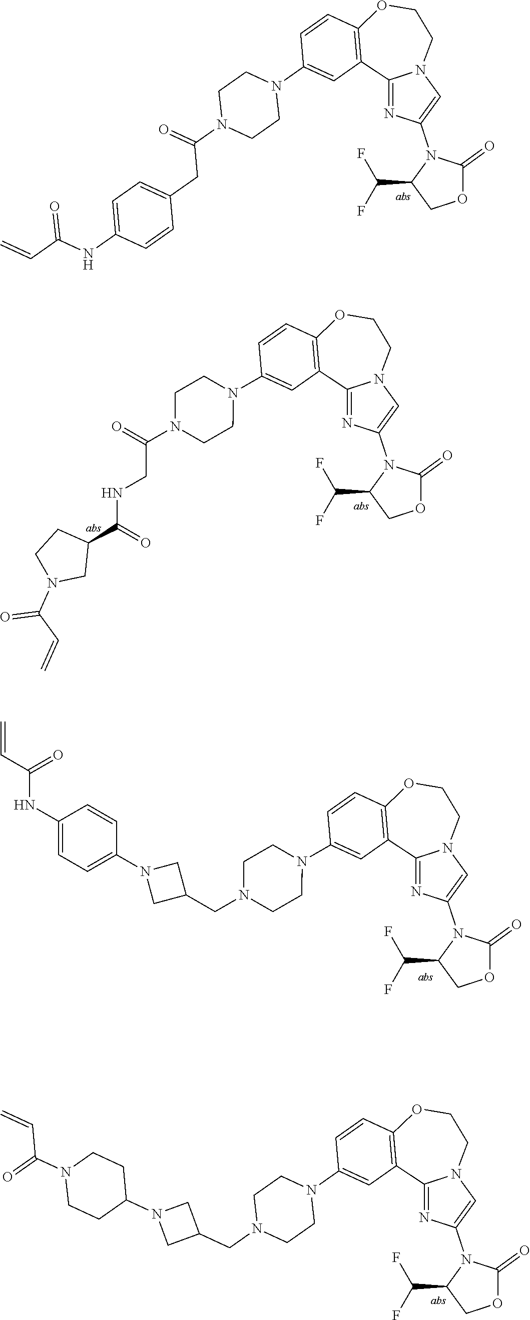 [SMILES: C=CC(=O)Nc1ccc(CC(=O)N2CCN(c3ccc4c(c3)-c3nc(N5C(=O)OC[C@H]5C(F)F)cn3CCO4)CC2)cc1 ; C=CC(=O)N1CC[C@@H](C(=O)NCC(=O)N2CCN(c3ccc4c(c3)-c3nc(N5C(=O)OC[C@H]5C(F)F)cn3CCO4)CC2)C1 ; C=CC(=O)Nc1ccc(N2CC(CN3CCN(c4ccc5c(c4)-c4nc(N6C(=O)OC[C@H]6C(F)F)cn4CCO5)CC3)C2)cc1 ; C=CC(=O)N1CCC(N2CC(CN3CCN(c4ccc5c(c4)-c4nc(N6C(=O)OC[C@H]6C(F)F)cn4CCO5)CC3)C2)CC1]

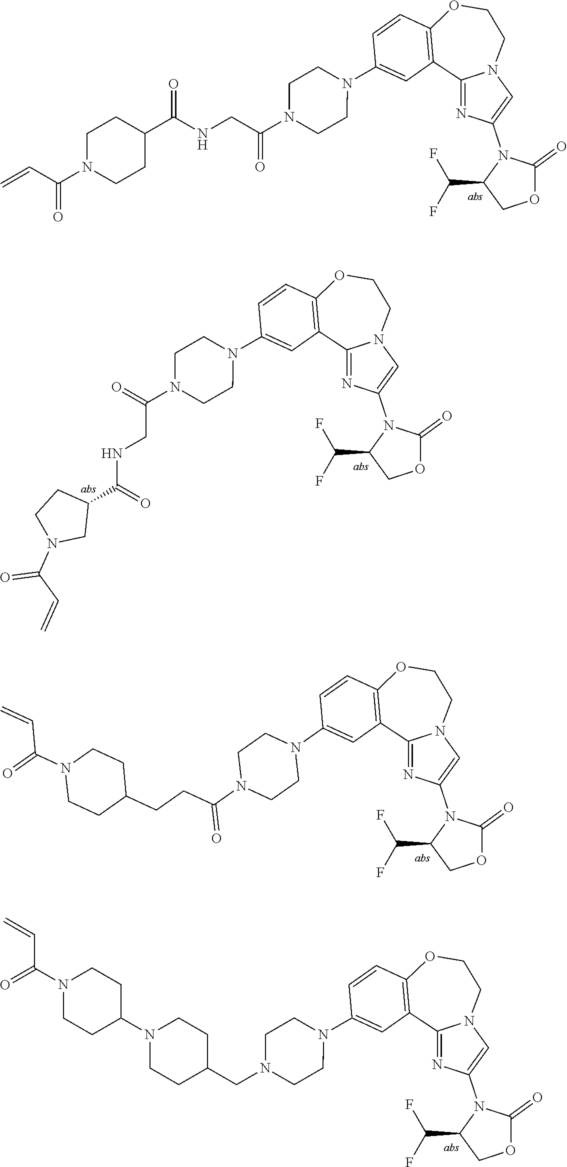 [SMILES: C=CC(=O)N1CCC(C(=O)NCC(=O)N2CCN(c3ccc4c(c3)-c3nc(N5C(=O)OC[C@H]5C(F)F)cn3CCO4)CC2)CC1 ; C=CC(=O)N1CC[C@H](C(=O)NCC(=O)N2CCN(c3ccc4c(c3)-c3nc(N5C(=O)OC[C@H]5C(F)F)cn3CCO4)CC2)C1 ; C=CC(=O)N1CCC(CCC(=O)N2CCN(c3ccc4c(c3)-c3nc(N5C(=O)OC[C@H]5C(F)F)cn3CCO4)CC2)CC1 ; C=CC(=O)N1CCC(N2CCC(CN3CCN(c4ccc5c(c4)-c4nc(N6C(=O)OC[C@H]6C(F)F)cn4CCO5)CC3)CC2)CC1]

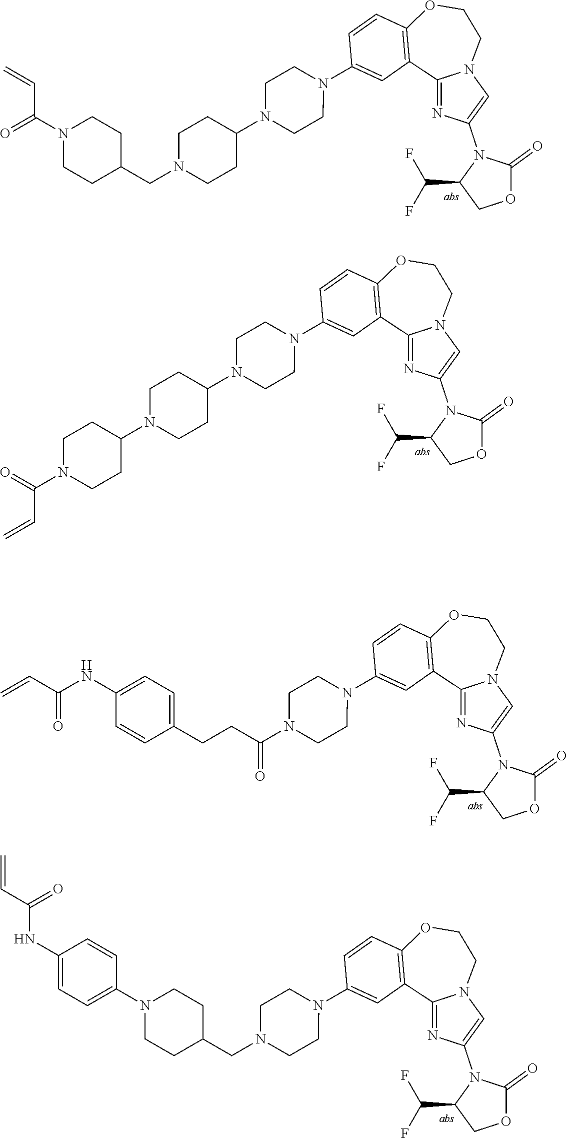 [SMILES: C=CC(=O)N1CCC(CN2CCC(N3CCN(c4ccc5c(c4)-c4nc(N6C(=O)OC[C@H]6C(F)F)cn4CCO5)CC3)CC2)CC1 ; C=CC(=O)N1CCC(N2CCC(N3CCN(c4ccc5c(c4)-c4nc(N6C(=O)OC[C@H]6C(F)F)cn4CCO5)CC3)CC2)CC1 ; C=CC(=O)Nc1ccc(CCC(=O)N2CCN(c3ccc4c(c3)-c3nc(N5C(=O)OC[C@H]5C(F)F)cn3CCO4)CC2)cc1 ; C=CC(=O)Nc1ccc(N2CCC(CN3CCN(c4ccc5c(c4)-c4nc(N6C(=O)OC[C@H]6C(F)F)cn4CCO5)CC3)CC2)cc1]

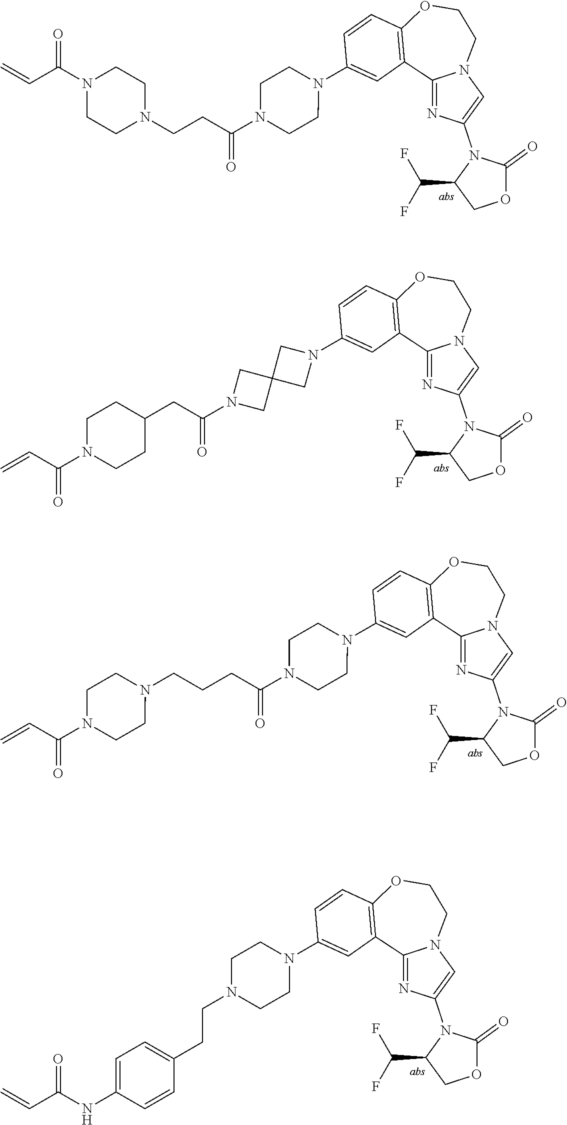 [SMILES: C=CC(=O)N1CCN(CCC(=O)N2CCN(c3ccc4c(c3)-c3nc(N5C(=O)OC[C@H]5C(F)F)cn3CCO4)CC2)CC1 ; C=CC(=O)N1CCC(CC(=O)N2CC3(C2)CN(c2ccc4c(c2)-c2nc(N5C(=O)OC[C@H]5C(F)F)cn2CCO4)C3)CC1 ; C=CC(=O)N1CCN(CCCC(=O)N2CCN(c3ccc4c(c3)-c3nc(N5C(=O)OC[C@H]5C(F)F)cn3CCO4)CC2)CC1 ; C=CC(=O)Nc1ccc(CCN2CCN(c3ccc4c(c3)-c3nc(N5C(=O)OC[C@H]5C(F)F)cn3CCO4)CC2)cc1]

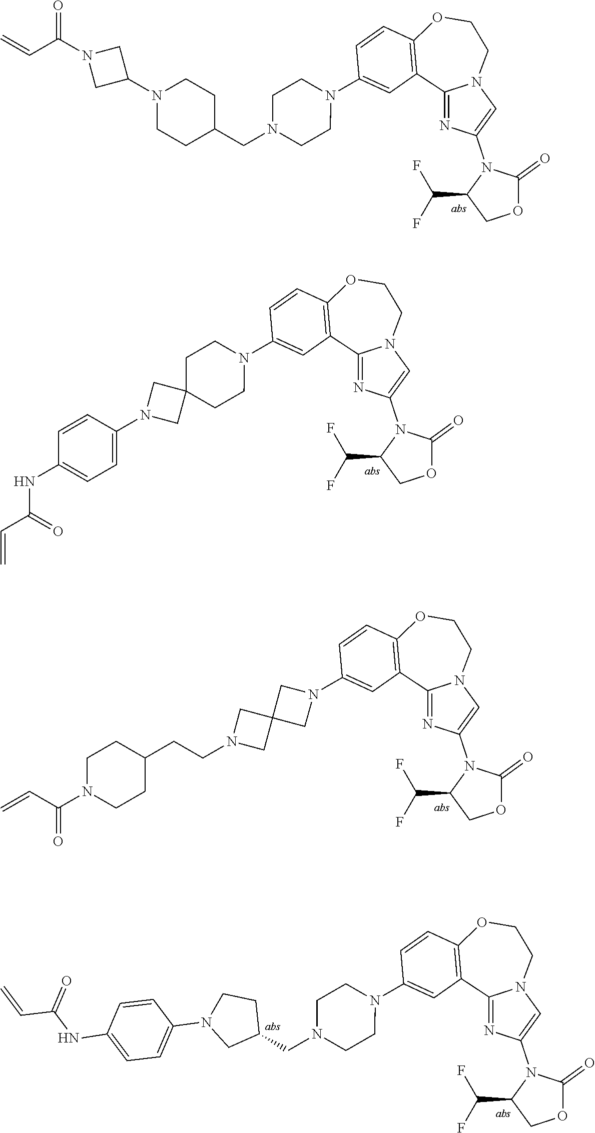 [SMILES: C=CC(=O)N1CC(N2CCC(CN3CCN(c4ccc5c(c4)-c4nc(N6C(=O)OC[C@H]6C(F)F)cn4CCO5)CC3)CC2)C1 ; C=CC(=O)Nc1ccc(N2CC3(CCN(c4ccc5c(c4)-c4nc(N6C(=O)OC[C@H]6C(F)F)cn4CCO5)CC3)C2)cc1 ; C=CC(=O)N1CCC(CCN2CC3(C2)CN(c2ccc4c(c2)-c2nc(N5C(=O)OC[C@H]5C(F)F)cn2CCO4)C3)CC1 ; C=CC(=O)Nc1ccc(N2CC[C@H](CN3CCN(c4ccc5c(c4)-c4nc(N6C(=O)OC[C@H]6C(F)F)cn4CCO5)CC3)C2)cc1]

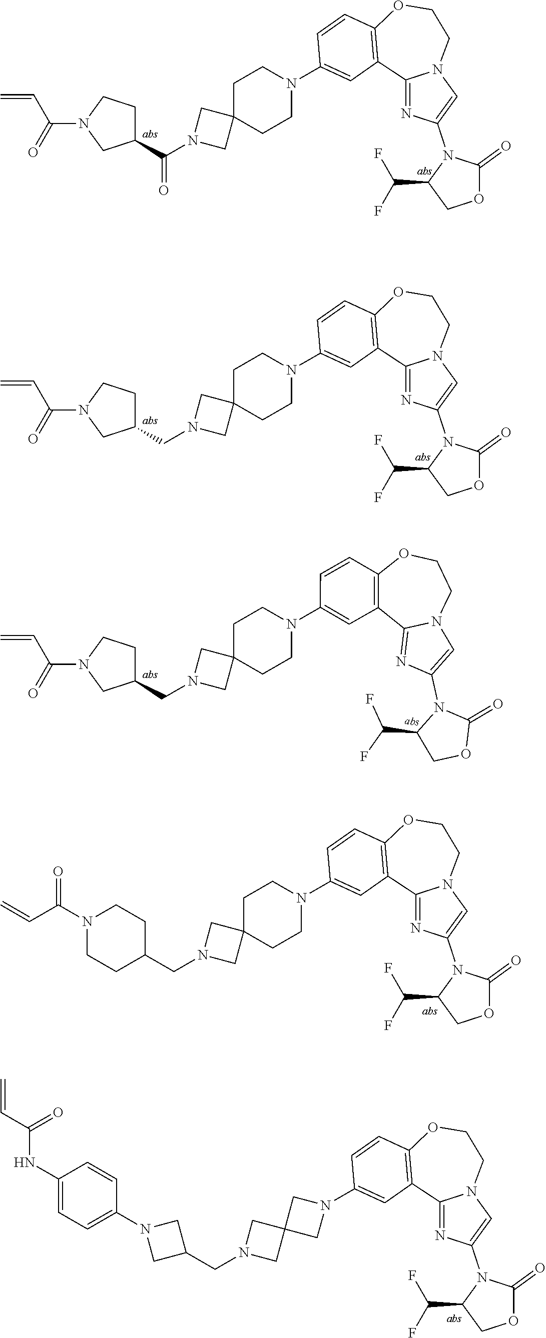 [SMILES: C=CC(=O)N1CC[C@@H](C(=O)N2CC3(CCN(c4ccc5c(c4)-c4nc(N6C(=O)OC[C@H]6C(F)F)cn4CCO5)CC3)C2)C1 ; C=CC(=O)N1CC[C@H](CN2CC3(CCN(c4ccc5c(c4)-c4nc(N6C(=O)OC[C@H]6C(F)F)cn4CCO5)CC3)C2)C1 ; C=CC(=O)N1CC[C@@H](CN2CC3(CCN(c4ccc5c(c4)-c4nc(N6C(=O)OC[C@H]6C(F)F)cn4CCO5)CC3)C2)C1 ; C=CC(=O)N1CCC(CN2CC3(CCN(c4ccc5c(c4)-c4nc(N6C(=O)OC[C@H]6C(F)F)cn4CCO5)CC3)C2)CC1 ; C=CC(=O)Nc1ccc(N2CC(CN3CC4(C3)CN(c3ccc5c(c3)-c3nc(N6C(=O)OC[C@H]6C(F)F)cn3CCO5)C4)C2)cc1]

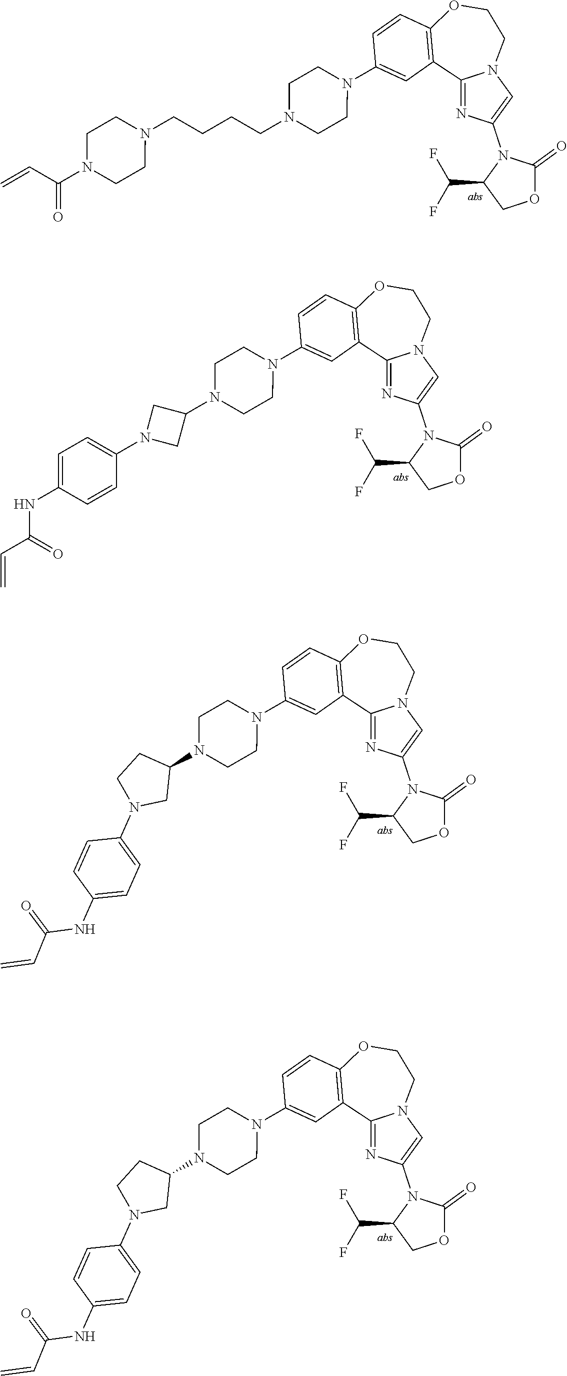 [SMILES: C=CC(=O)N1CCN(CCCCN2CCN(c3ccc4c(c3)-c3nc(N5C(=O)OC[C@H]5C(F)F)cn3CCO4)CC2)CC1 ; C=CC(=O)Nc1ccc(N2CC(N3CCN(c4ccc5c(c4)-c4nc(N6C(=O)OC[C@H]6C(F)F)cn4CCO5)CC3)C2)cc1 ; C=CC(=O)Nc1ccc(N2CC[C@@H](N3CCN(c4ccc5c(c4)-c4nc(N6C(=O)OC[C@H]6C(F)F)cn4CCO5)CC3)C2)cc1 ; C=CC(=O)Nc1ccc(N2CC[C@H](N3CCN(c4ccc5c(c4)-c4nc(N6C(=O)OC[C@H]6C(F)F)cn4CCO5)CC3)C2)cc1]

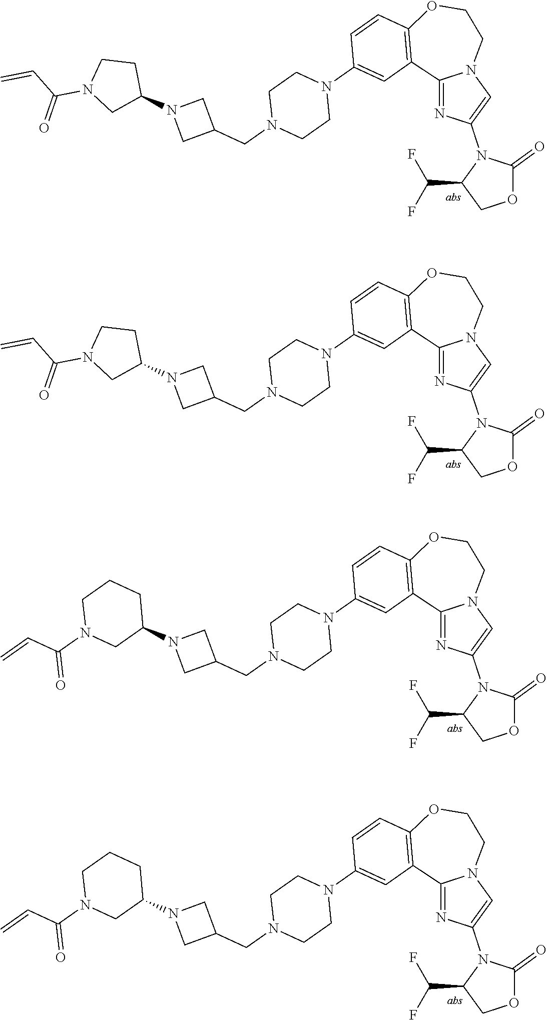 [SMILES: C=CC(=O)N1CC[C@@H](N2CC(CN3CCN(c4ccc5c(c4)-c4nc(N6C(=O)OC[C@H]6C(F)F)cn4CCO5)CC3)C2)C1 ; C=CC(=O)N1CC[C@H](N2CC(CN3CCN(c4ccc5c(c4)-c4nc(N6C(=O)OC[C@H]6C(F)F)cn4CCO5)CC3)C2)C1 ; C=CC(=O)N1CCC[C@@H](N2CC(CN3CCN(c4ccc5c(c4)-c4nc(N6C(=O)OC[C@H]6C(F)F)cn4CCO5)CC3)C2)C1 ; C=CC(=O)N1CCC[C@H](N2CC(CN3CCN(c4ccc5c(c4)-c4nc(N6C(=O)OC[C@H]6C(F)F)cn4CCO5)CC3)C2)C1]

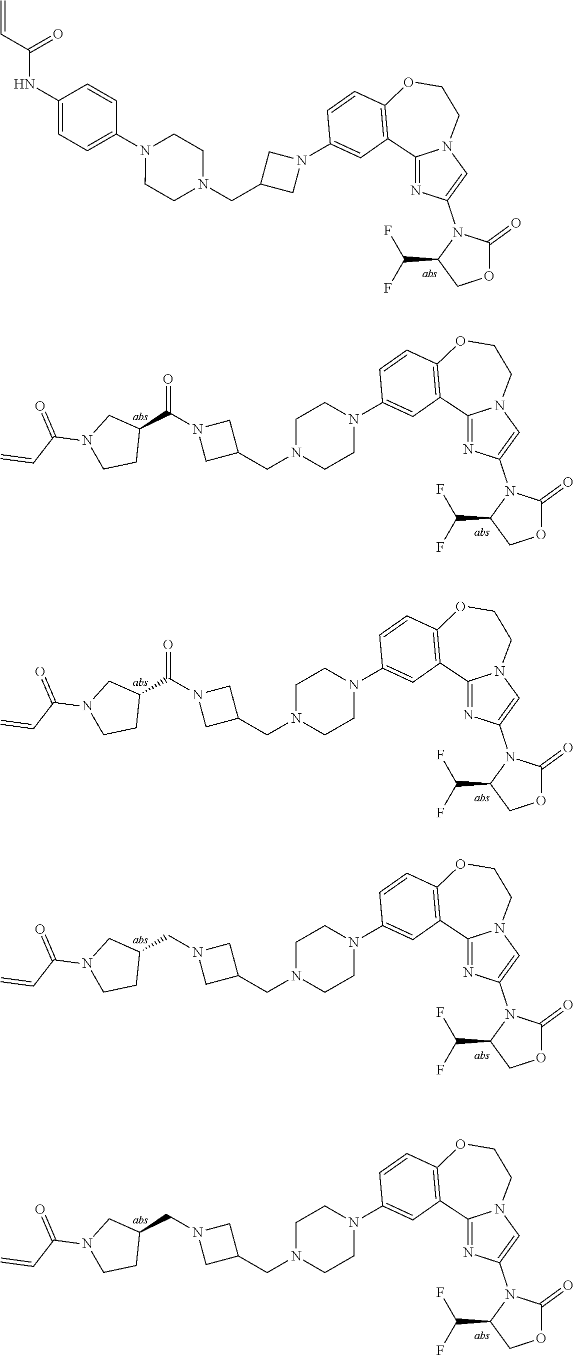 [SMILES: C=CC(=O)Nc1ccc(N2CCN(CC3CN(c4ccc5c(c4)-c4nc(N6C(=O)OC[C@H]6C(F)F)cn4CCO5)C3)CC2)cc1 ; C=CC(=O)N1CC[C@H](C(=O)N2CC(CN3CCN(c4ccc5c(c4)-c4nc(N6C(=O)OC[C@H]6C(F)F)cn4CCO5)CC3)C2)C1 ; C=CC(=O)N1CC[C@@H](C(=O)N2CC(CN3CCN(c4ccc5c(c4)-c4nc(N6C(=O)OC[C@H]6C(F)F)cn4CCO5)CC3)C2)C1 ; C=CC(=O)N1CC[C@@H](CN2CC(CN3CCN(c4ccc5c(c4)-c4nc(N6C(=O)OC[C@H]6C(F)F)cn4CCO5)CC3)C2)C1 ; C=CC(=O)N1CC[C@H](CN2CC(CN3CCN(c4ccc5c(c4)-c4nc(N6C(=O)OC[C@H]6C(F)F)cn4CCO5)CC3)C2)C1]

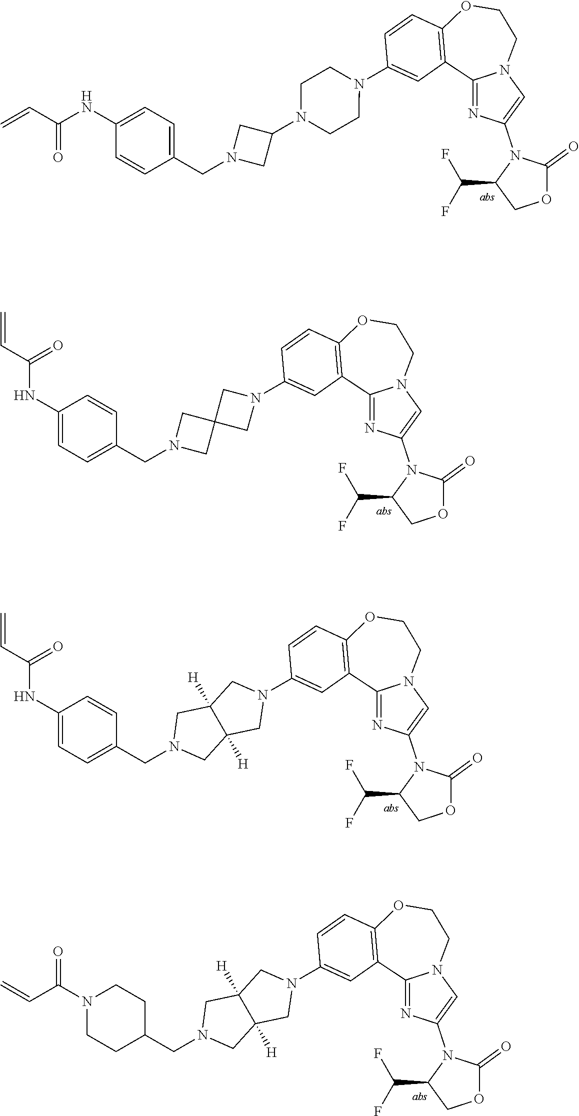 [SMILES: C=CC(=O)Nc1ccc(CN2CC(N3CCN(c4ccc5c(c4)-c4nc(N6C(=O)OC[C@H]6C(F)F)cn4CCO5)CC3)C2)cc1 ; C=CC(=O)Nc1ccc(CN2CC3(C2)CN(c2ccc4c(c2)-c2nc(N5C(=O)OC[C@H]5C(F)F)cn2CCO4)C3)cc1 ; C=CC(=O)Nc1ccc(CN2C[C@@H]3CN(c4ccc5c(c4)-c4nc(N6C(=O)OC[C@H]6C(F)F)cn4CCO5)C[C@@H]3C2)cc1 ; C=CC(=O)N1CCC(CN2C[C@@H]3CN(c4ccc5c(c4)-c4nc(N6C(=O)OC[C@H]6C(F)F)cn4CCO5)C[C@@H]3C2)CC1]

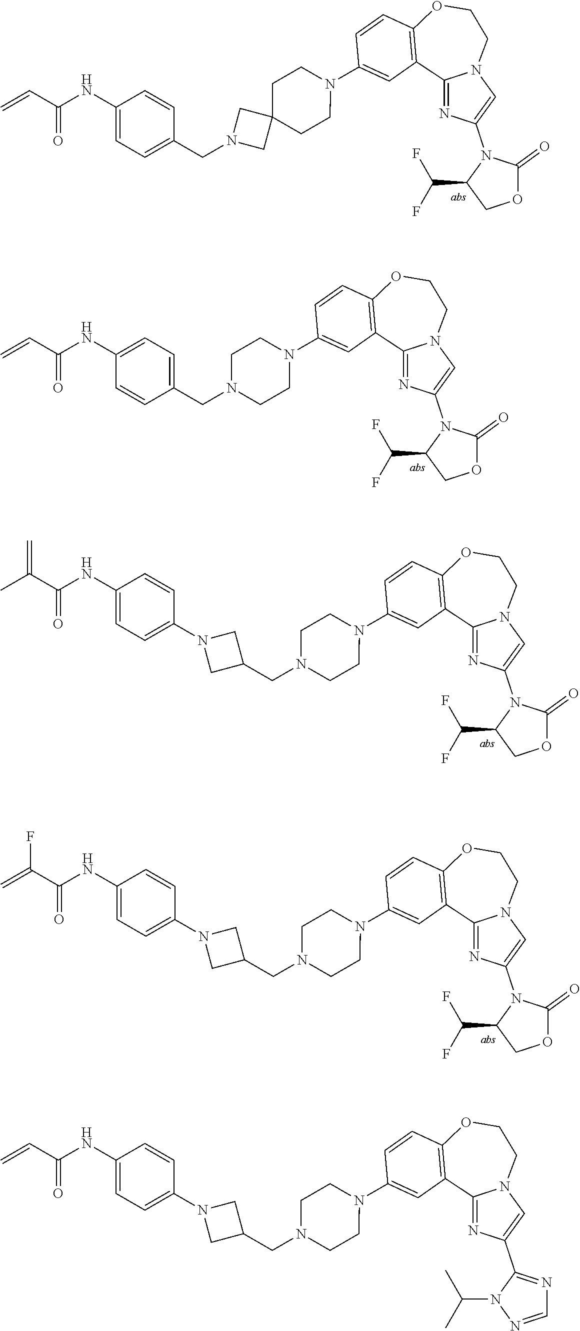 [SMILES: C=CC(=O)Nc1ccc(CN2CC3(CCN(c4ccc5c(c4)-c4nc(N6C(=O)OC[C@H]6C(F)F)cn4CCO5)CC3)C2)cc1 ; C=CC(=O)Nc1ccc(CN2CCN(c3ccc4c(c3)-c3nc(N5C(=O)OC[C@H]5C(F)F)cn3CCO4)CC2)cc1 ; C=C(C)C(=O)Nc1ccc(N2CC(CN3CCN(c4ccc5c(c4)-c4nc(N6C(=O)OC[C@H]6C(F)F)cn4CCO5)CC3)C2)cc1 ; C=C(F)C(=O)Nc1ccc(N2CC(CN3CCN(c4ccc5c(c4)-c4nc(N6C(=O)OC[C@H]6C(F)F)cn4CCO5)CC3)C2)cc1 ; C=CC(=O)Nc1ccc(N2CC(CN3CCN(c4ccc5c(c4)-c4nc(-c6ncnn6C(C)C)cn4CCO5)CC3)C2)cc1]

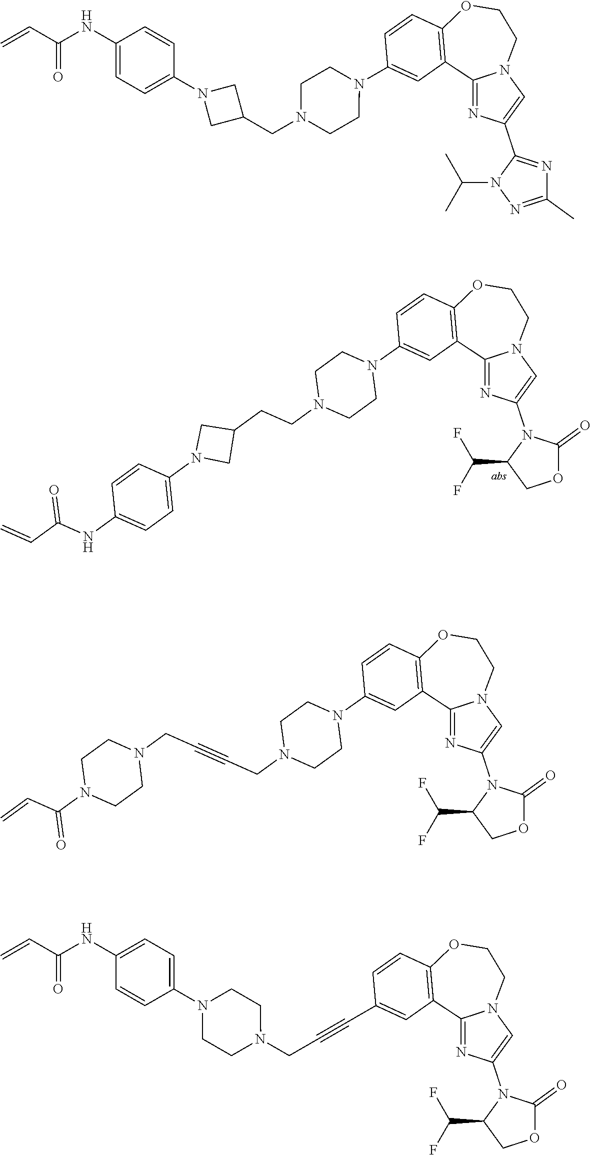

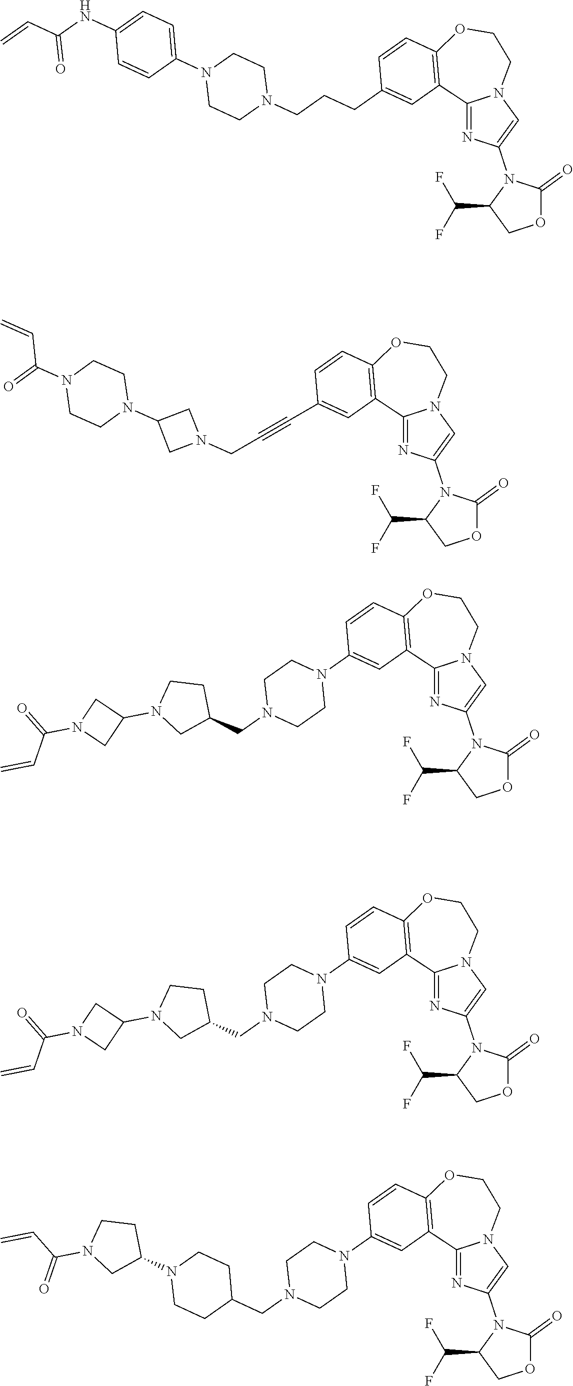 [SMILES: C=CC(=O)Nc1ccc(N2CCN(CCCc3ccc4c(c3)-c3nc(N5C(=O)OC[C@H]5C(F)F)cn3CCO4)CC2)cc1 ; C=CC(=O)N1CCN(C2CN(CC#Cc3ccc4c(c3)-c3nc(N5C(=O)OC[C@H]5C(F)F)cn3CCO4)C2)CC1 ; C=CC(=O)N1CC(N2CC[C@@H](CN3CCN(c4ccc5c(c4)-c4nc(N6C(=O)OC[C@H]6C(F)F)cn4CCO5)CC3)C2)C1 ; C=CC(=O)N1CC(N2CC[C@H](CN3CCN(c4ccc5c(c4)-c4nc(N6C(=O)OC[C@H]6C(F)F)cn4CCO5)CC3)C2)C1 ; C=CC(=O)N1CC[C@H](N2CCC(CN3CCN(c4ccc5c(c4)-c4nc(N6C(=O)OC[C@H]6C(F)F)cn4CCO5)CC3)CC2)C1]

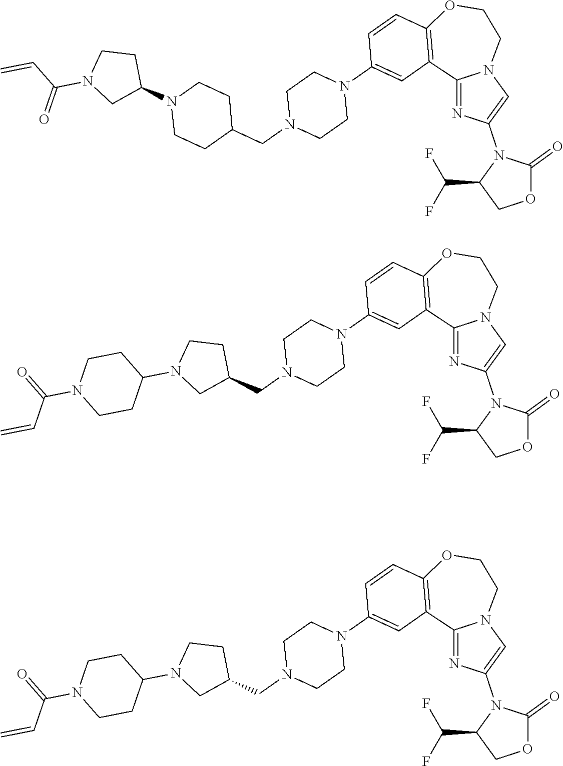

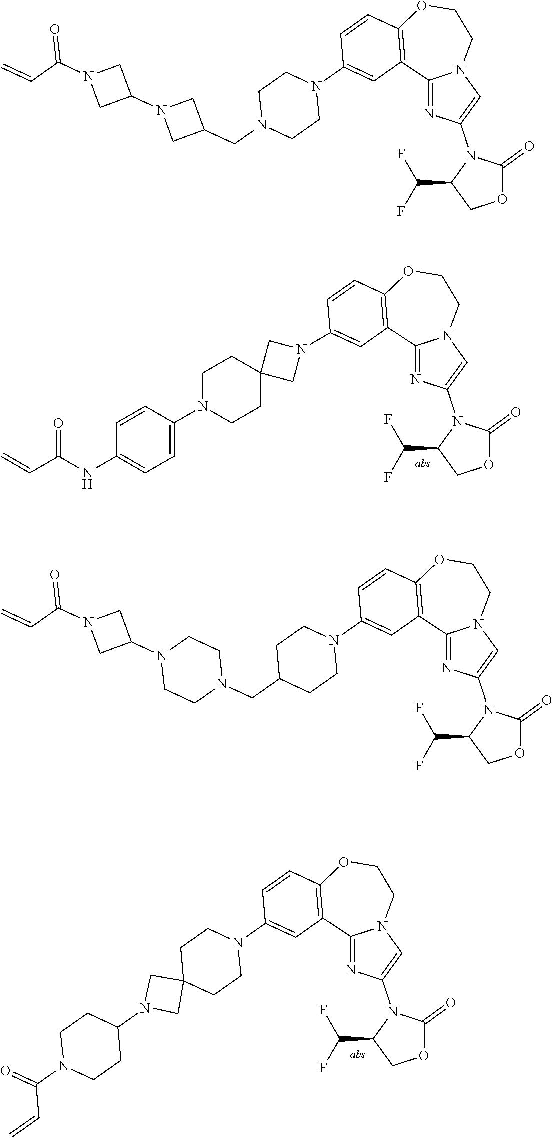 [SMILES: C=CC(=O)N1CC(N2CC(CN3CCN(c4ccc5c(c4)-c4nc(N6C(=O)OC[C@H]6C(F)F)cn4CCO5)CC3)C2)C1 ; C=CC(=O)Nc1ccc(N2CCC3(CC2)CN(c2ccc4c(c2)-c2nc(N5C(=O)OC[C@H]5C(F)F)cn2CCO4)C3)cc1 ; C=CC(=O)N1CC(N2CCN(CC3CCN(c4ccc5c(c4)-c4nc(N6C(=O)OC[C@H]6C(F)F)cn4CCO5)CC3)CC2)C1 ; C=CC(=O)N1CCC(N2CC3(CCN(c4ccc5c(c4)-c4nc(N6C(=O)OC[C@H]6C(F)F)cn4CCO5)CC3)C2)CC1]

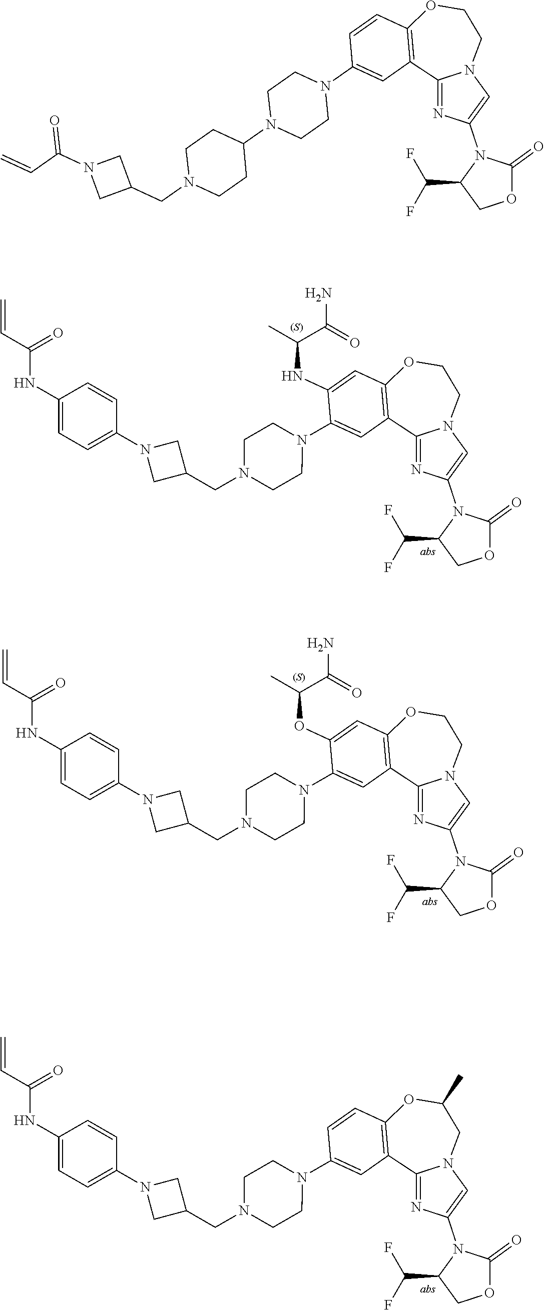

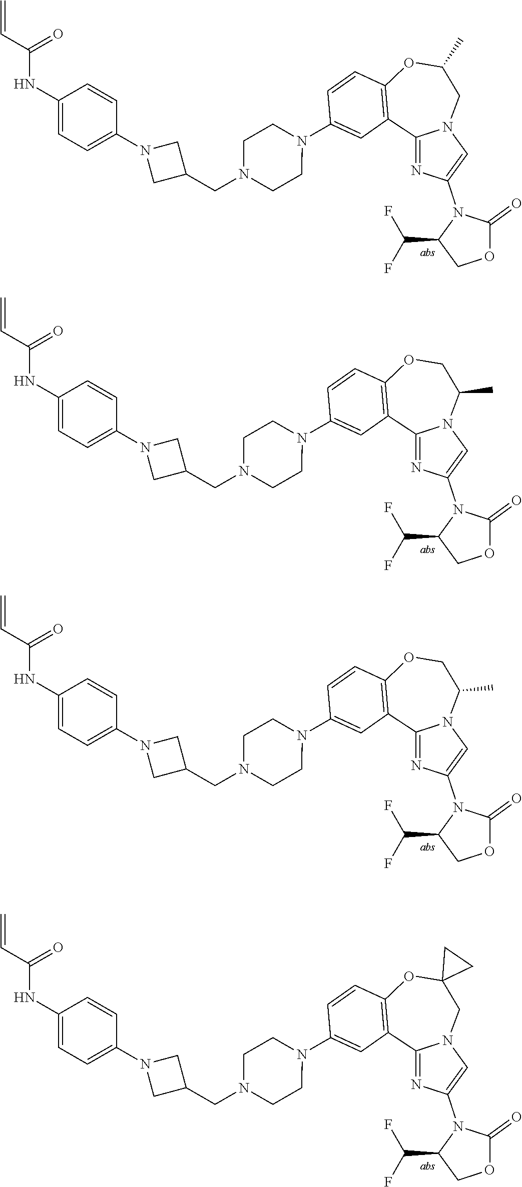 [SMILES: C=CC(=O)Nc1ccc(N2CC(CN3CCN(c4ccc5c(c4)-c4nc(N6C(=O)OC[C@H]6C(F)F)cn4C[C@@H](C)O5)CC3)C2)cc1 ; C=CC(=O)Nc1ccc(N2CC(CN3CCN(c4ccc5c(c4)-c4nc(N6C(=O)OC[C@H]6C(F)F)cn4[C@H](C)CO5)CC3)C2)cc1 ; C=CC(=O)Nc1ccc(N2CC(CN3CCN(c4ccc5c(c4)-c4nc(N6C(=O)OC[C@H]6C(F)F)cn4[C@@H](C)CO5)CC3)C2)cc1 ; C=CC(=O)Nc1ccc(N2CC(CN3CCN(c4ccc5c(c4)-c4nc(N6C(=O)OC[C@H]6C(F)F)cn4CC4(CC4)O5)CC3)C2)cc1]

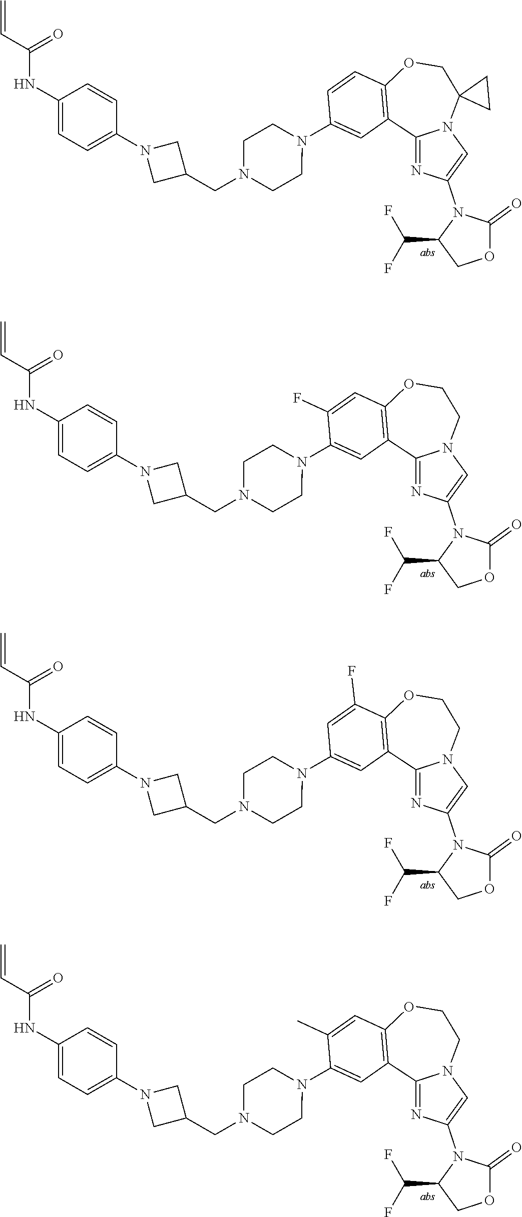 [SMILES: C=CC(=O)Nc1ccc(N2CC(CN3CCN(c4ccc5c(c4)-c4nc(N6C(=O)OC[C@H]6C(F)F)cn4C4(CC4)CO5)CC3)C2)cc1 ; C=CC(=O)Nc1ccc(N2CC(CN3CCN(c4cc5c(cc4F)OCCn4cc(N6C(=O)OC[C@H]6C(F)F)nc4-5)CC3)C2)cc1 ; C=CC(=O)Nc1ccc(N2CC(CN3CCN(c4cc(F)c5c(c4)-c4nc(N6C(=O)OC[C@H]6C(F)F)cn4CCO5)CC3)C2)cc1 ; C=CC(=O)Nc1ccc(N2CC(CN3CCN(c4cc5c(cc4C)OCCn4cc(N6C(=O)OC[C@H]6C(F)F)nc4-5)CC3)C2)cc1]

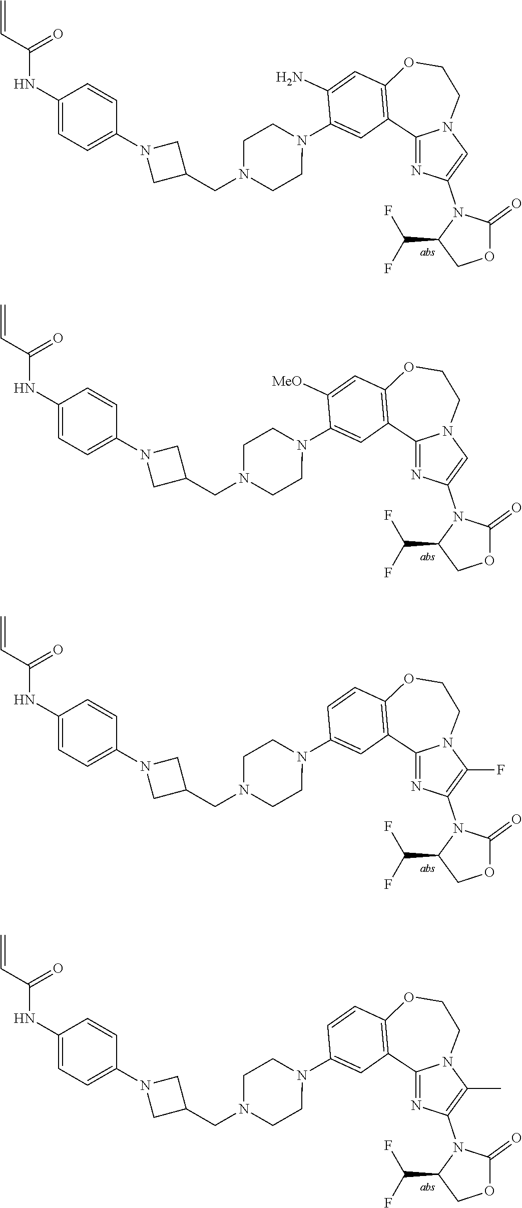 [SMILES: C=CC(=O)Nc1ccc(N2CC(CN3CCN(c4cc5c(cc4N)OCCn4cc(N6C(=O)OC[C@H]6C(F)F)nc4-5)CC3)C2)cc1 ; C=CC(=O)Nc1ccc(N2CC(CN3CCN(c4cc5c(cc4OC)OCCn4cc(N6C(=O)OC[C@H]6C(F)F)nc4-5)CC3)C2)cc1 ; C=CC(=O)Nc1ccc(N2CC(CN3CCN(c4ccc5c(c4)-c4nc(N6C(=O)OC[C@H]6C(F)F)c(F)n4CCO5)CC3)C2)cc1 ; C=CC(=O)Nc1ccc(N2CC(CN3CCN(c4ccc5c(c4)-c4nc(N6C(=O)OC[C@H]6C(F)F)c(C)n4CCO5)CC3)C2)cc1]

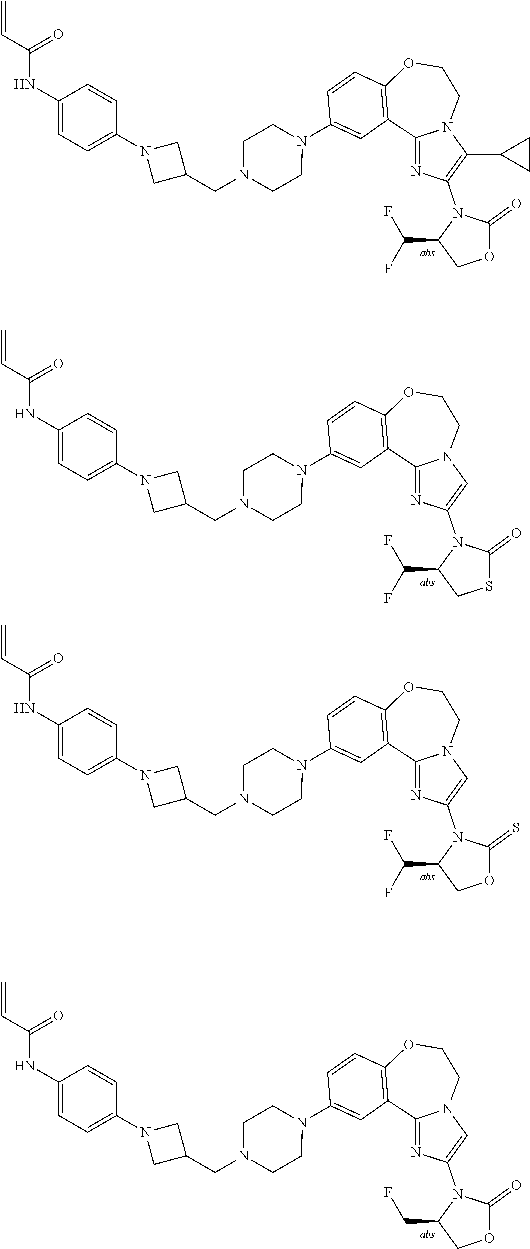 [SMILES: C=CC(=O)Nc1ccc(N2CC(CN3CCN(c4ccc5c(c4)-c4nc(N6C(=O)OC[C@H]6C(F)F)c(C6CC6)n4CCO5)CC3)C2)cc1 ; C=CC(=O)Nc1ccc(N2CC(CN3CCN(c4ccc5c(c4)-c4nc(N6C(=O)SC[C@H]6C(F)F)cn4CCO5)CC3)C2)cc1 ; C=CC(=O)Nc1ccc(N2CC(CN3CCN(c4ccc5c(c4)-c4nc(N6C(=S)OC[C@H]6C(F)F)cn4CCO5)CC3)C2)cc1 ; C=CC(=O)Nc1ccc(N2CC(CN3CCN(c4ccc5c(c4)-c4nc(N6C(=O)OC[C@H]6CF)cn4CCO5)CC3)C2)cc1]

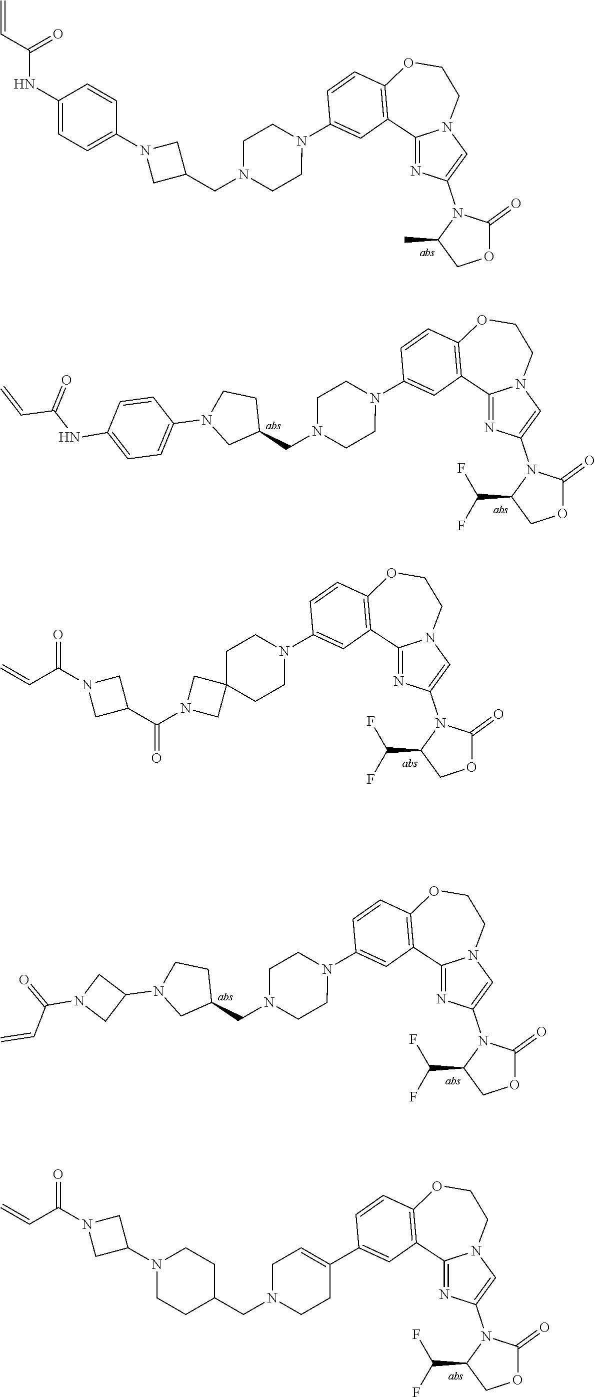 [SMILES: C=CC(=O)Nc1ccc(N2CC(CN3CCN(c4ccc5c(c4)-c4nc(N6C(=O)OC[C@H]6C)cn4CCO5)CC3)C2)cc1 ; C=CC(=O)Nc1ccc(N2CC[C@@H](CN3CCN(c4ccc5c(c4)-c4nc(N6C(=O)OC[C@H]6C(F)F)cn4CCO5)CC3)C2)cc1 ; C=CC(=O)N1CC(C(=O)N2CC3(CCN(c4ccc5c(c4)-c4nc(N6C(=O)OC[C@H]6C(F)F)cn4CCO5)CC3)C2)C1 ; C=CC(=O)N1CC(N2CC[C@@H](CN3CCN(c4ccc5c(c4)-c4nc(N6C(=O)OC[C@H]6C(F)F)cn4CCO5)CC3)C2)C1 ; C=CC(=O)N1CC(N2CCC(CN3CC=C(c4ccc5c(c4)-c4nc(N6C(=O)OC[C@H]6C(F)F)cn4CCO5)CC3)CC2)C1]

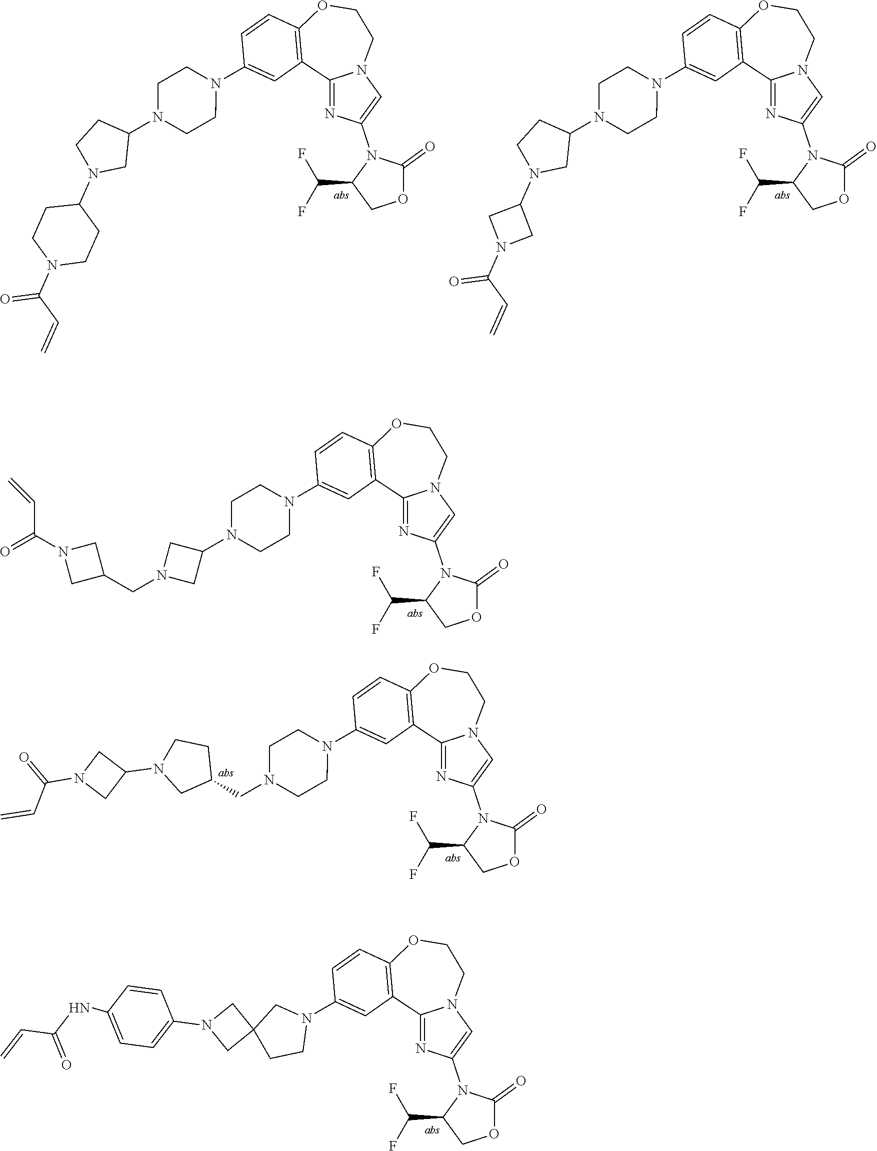

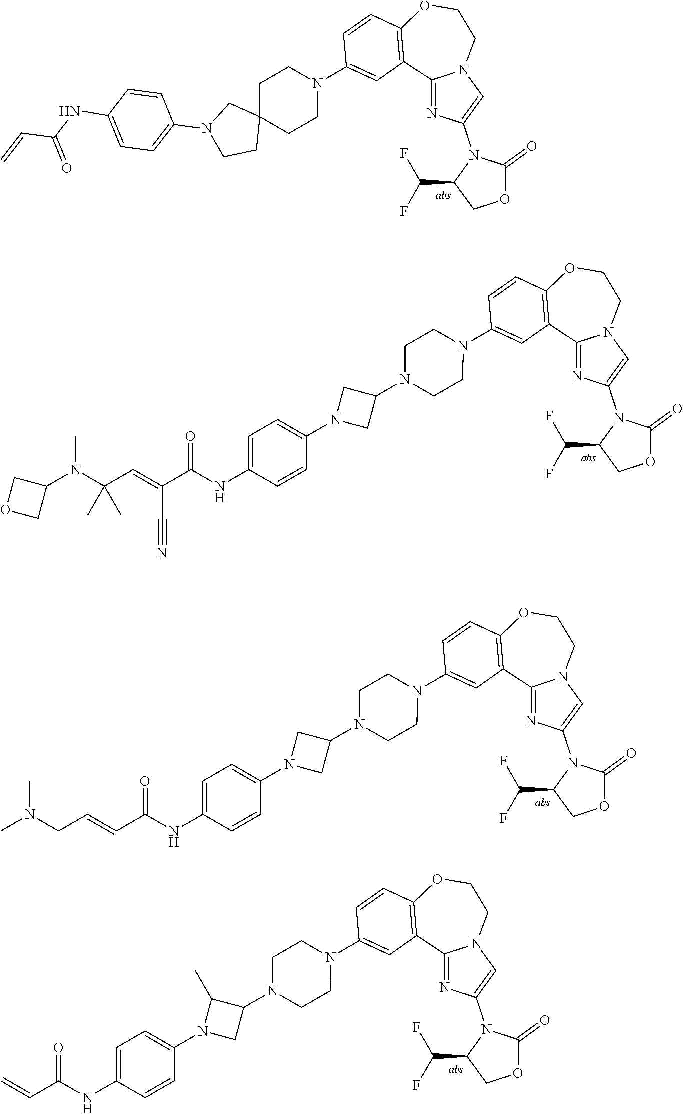 [SMILES: C=CC(=O)Nc1ccc(N2CCC3(CCN(c4ccc5c(c4)-c4nc(N6C(=O)OC[C@H]6C(F)F)cn4CCO5)CC3)C2)cc1 ; CN(C1COC1)C(C)(C)/C=C(\C#N)C(=O)Nc1ccc(N2CC(N3CCN(c4ccc5c(c4)-c4nc(N6C(=O)OC[C@H]6C(F)F)cn4CCO5)CC3)C2)cc1 ; CN(C)C/C=C/C(=O)Nc1ccc(N2CC(N3CCN(c4ccc5c(c4)-c4nc(N6C(=O)OC[C@H]6C(F)F)cn4CCO5)CC3)C2)cc1 ; C=CC(=O)Nc1ccc(N2CC(N3CCN(c4ccc5c(c4)-c4nc(N6C(=O)OC[C@H]6C(F)F)cn4CCO5)CC3)C2C)cc1]

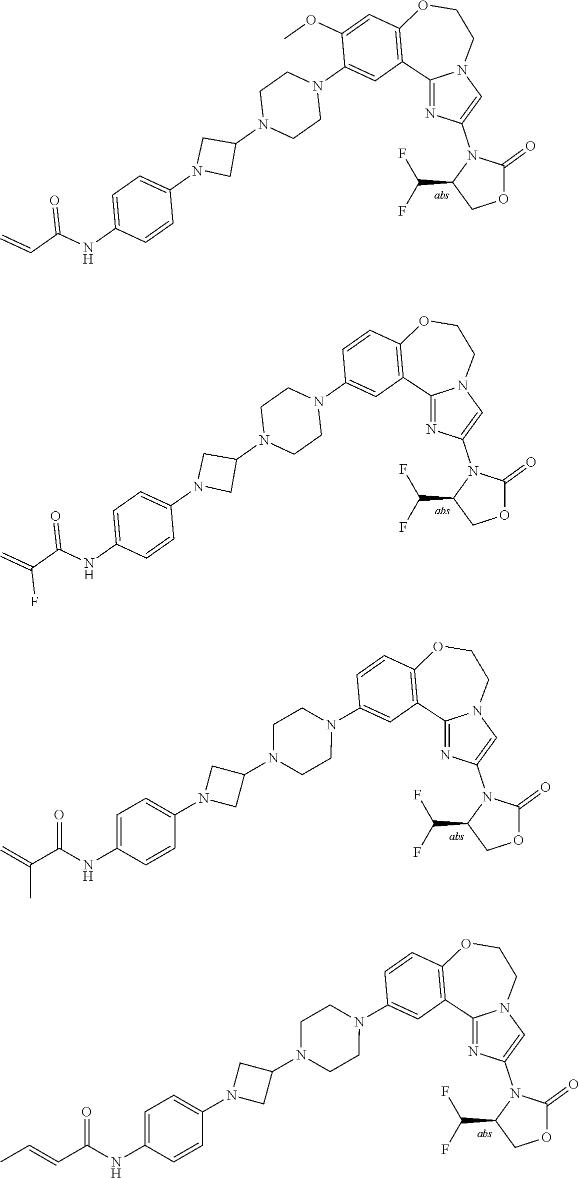 [SMILES: C=CC(=O)Nc1ccc(N2CC(N3CCN(c4cc5c(cc4OC)OCCn4cc(N6C(=O)OC[C@H]6C(F)F)nc4-5)CC3)C2)cc1 ; C=C(F)C(=O)Nc1ccc(N2CC(N3CCN(c4ccc5c(c4)-c4nc(N6C(=O)OC[C@H]6C(F)F)cn4CCO5)CC3)C2)cc1 ; C=C(C)C(=O)Nc1ccc(N2CC(N3CCN(c4ccc5c(c4)-c4nc(N6C(=O)OC[C@H]6C(F)F)cn4CCO5)CC3)C2)cc1 ; C/C=C/C(=O)Nc1ccc(N2CC(N3CCN(c4ccc5c(c4)-c4nc(N6C(=O)OC[C@H]6C(F)F)cn4CCO5)CC3)C2)cc1]

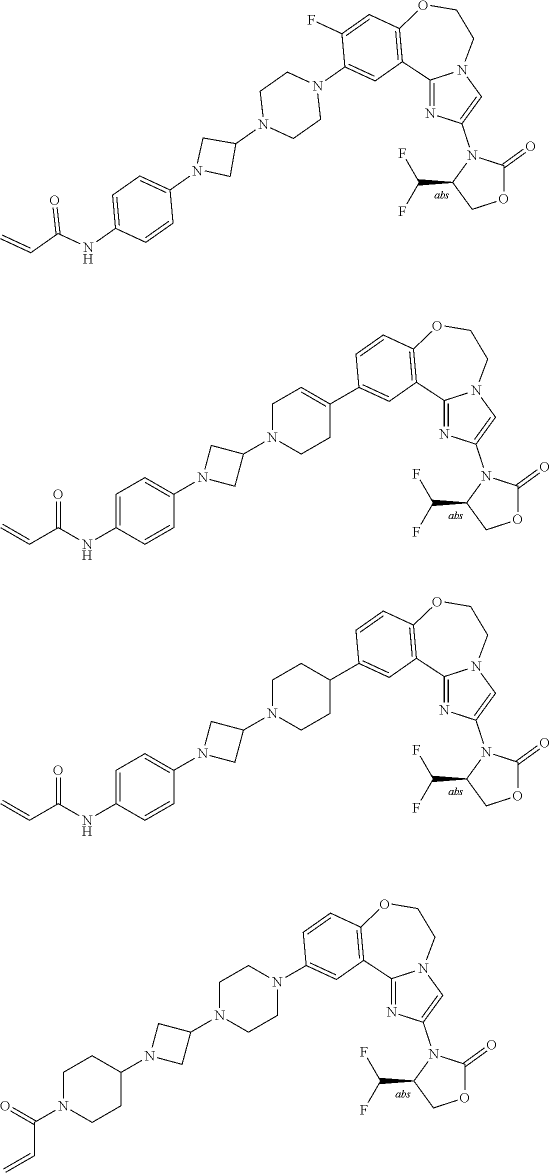 [SMILES: C=CC(=O)Nc1ccc(N2CC(N3CCN(c4cc5c(cc4F)OCCn4cc(N6C(=O)OC[C@H]6C(F)F)nc4-5)CC3)C2)cc1 ; C=CC(=O)Nc1ccc(N2CC(N3CC=C(c4ccc5c(c4)-c4nc(N6C(=O)OC[C@H]6C(F)F)cn4CCO5)CC3)C2)cc1 ; C=CC(=O)Nc1ccc(N2CC(N3CCC(c4ccc5c(c4)-c4nc(N6C(=O)OC[C@H]6C(F)F)cn4CCO5)CC3)C2)cc1 ; C=CC(=O)N1CCC(N2CC(N3CCN(c4ccc5c(c4)-c4nc(N6C(=O)OC[C@H]6C(F)F)cn4CCO5)CC3)C2)CC1]

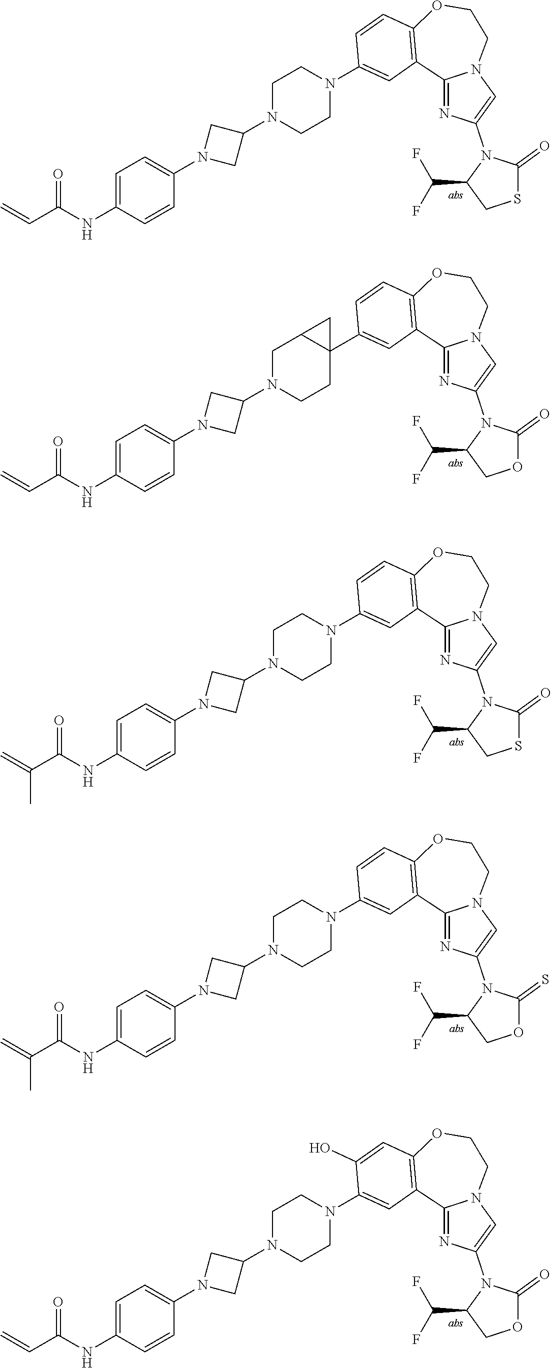 [SMILES: C=CC(=O)Nc1ccc(N2CC(N3CCN(c4ccc5c(c4)-c4nc(N6C(=O)SC[C@H]6C(F)F)cn4CCO5)CC3)C2)cc1 ; C=CC(=O)Nc1ccc(N2CC(N3CCC4(c5ccc6c(c5)-c5nc(N7C(=O)OC[C@H]7C(F)F)cn5CCO6)CC4C3)C2)cc1 ; C=C(C)C(=O)Nc1ccc(N2CC(N3CCN(c4ccc5c(c4)-c4nc(N6C(=O)SC[C@H]6C(F)F)cn4CCO5)CC3)C2)cc1 ; C=C(C)C(=O)Nc1ccc(N2CC(N3CCN(c4ccc5c(c4)-c4nc(N6C(=S)OC[C@H]6C(F)F)cn4CCO5)CC3)C2)cc1 ; C=CC(=O)Nc1ccc(N2CC(N3CCN(c4cc5c(cc4O)OCCn4cc(N6C(=O)OC[C@H]6C(F)F)nc4-5)CC3)C2)cc1]

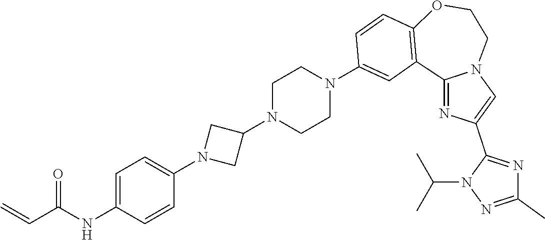 [SMILES: C=CC(=O)Nc1ccc(N2CC(N3CCN(c4ccc5c(c4)-c4nc(-c6nc(C)nn6C(C)C)cn4CCO5)CC3)C2)cc1]

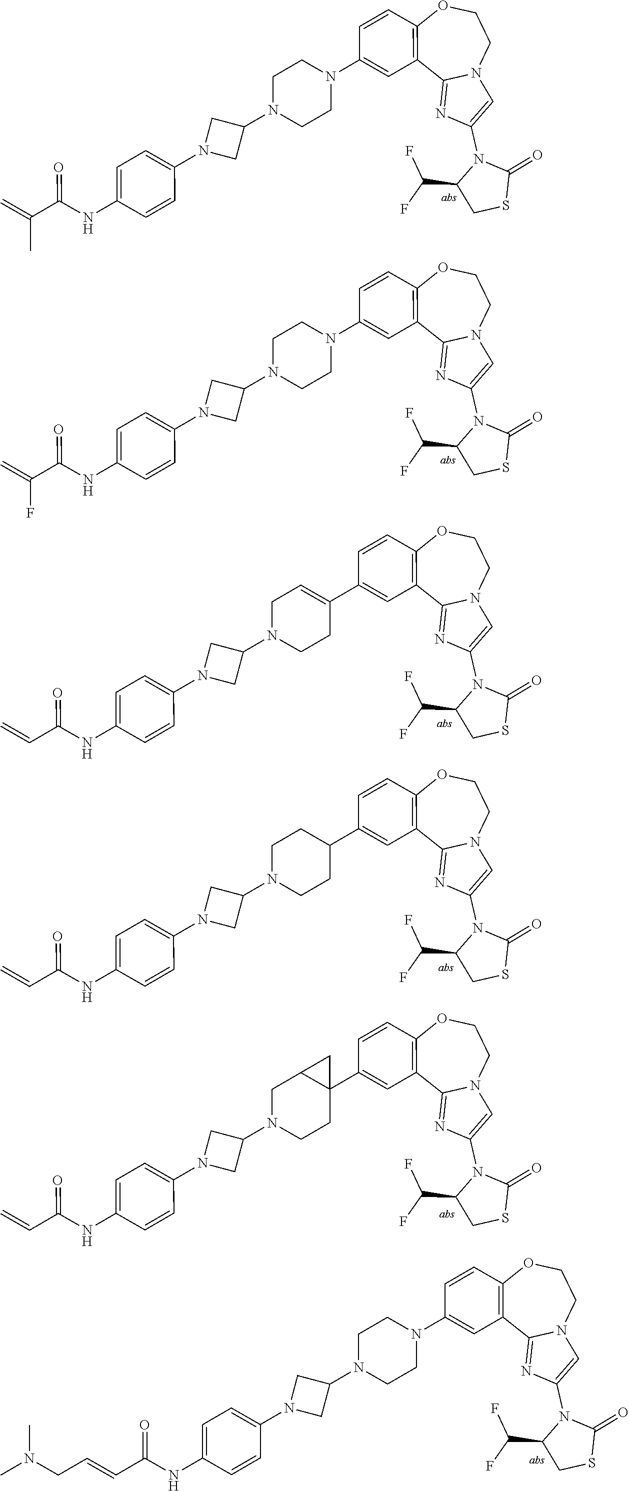

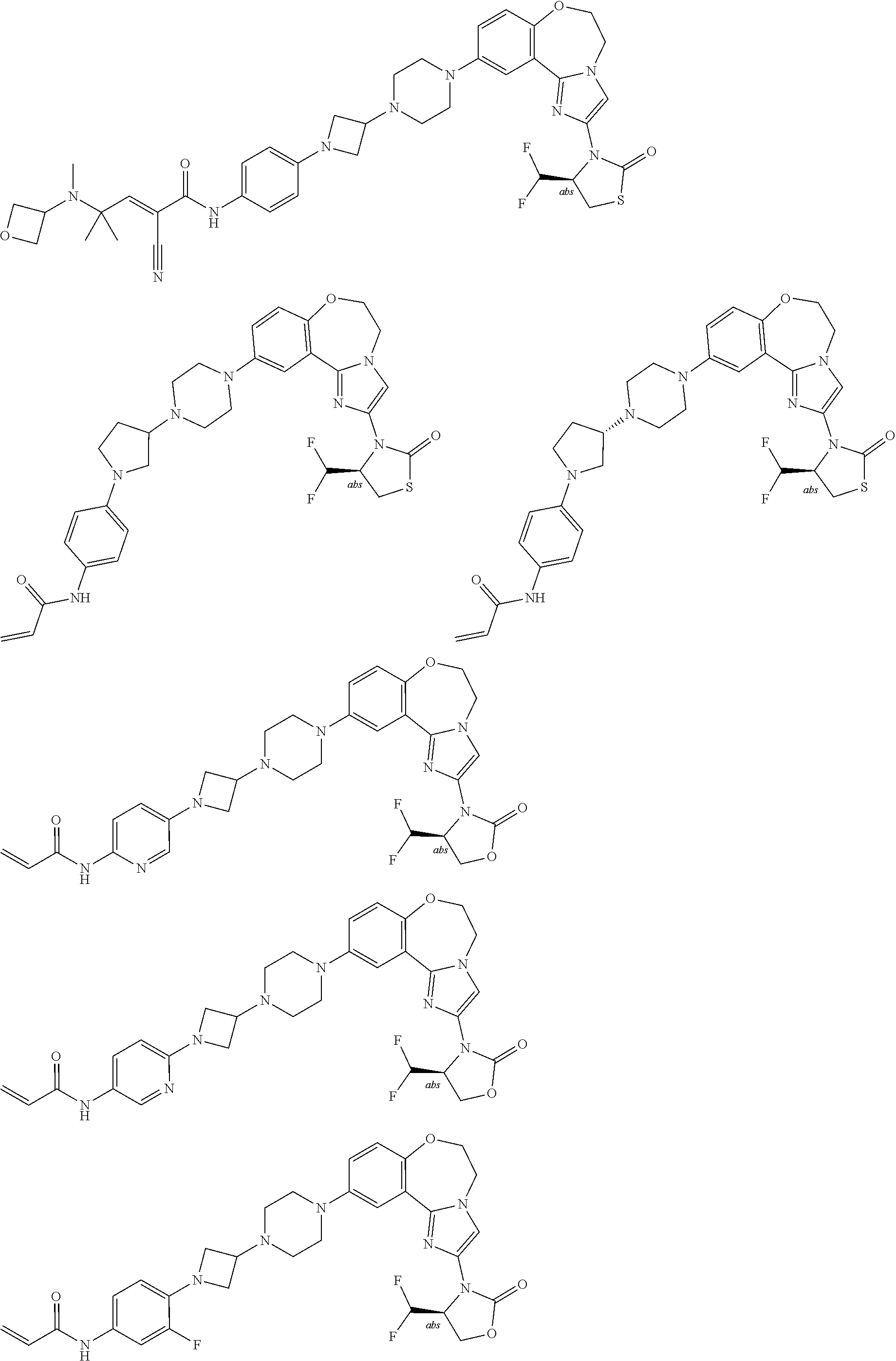

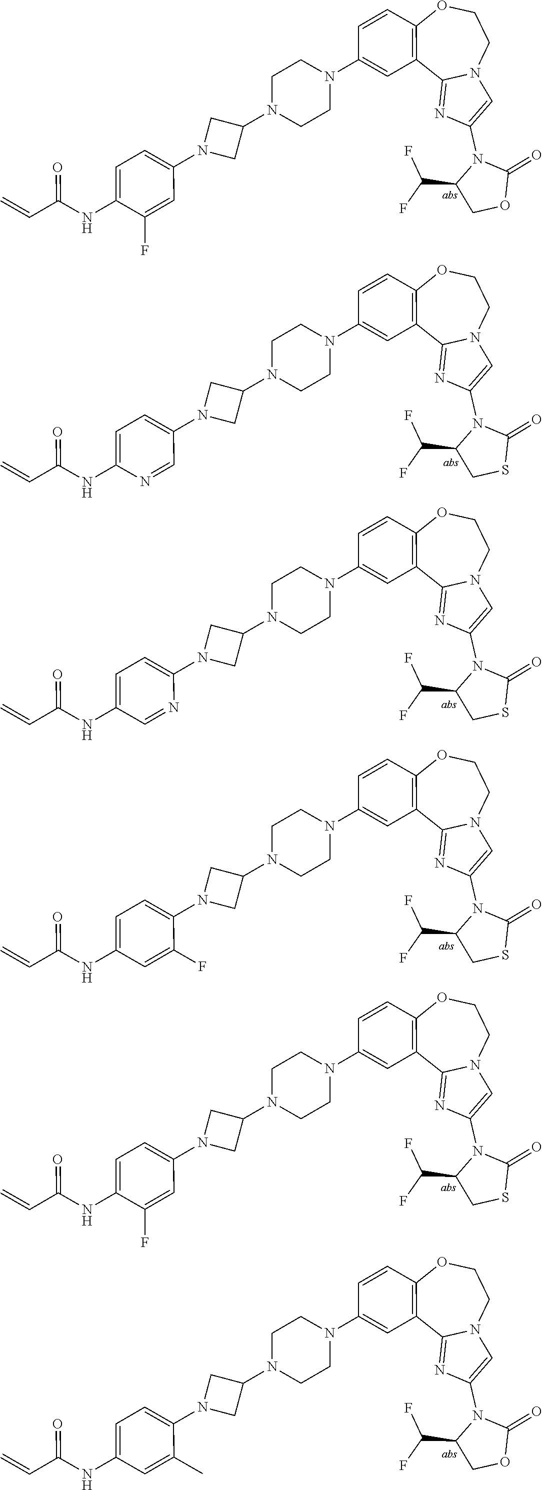

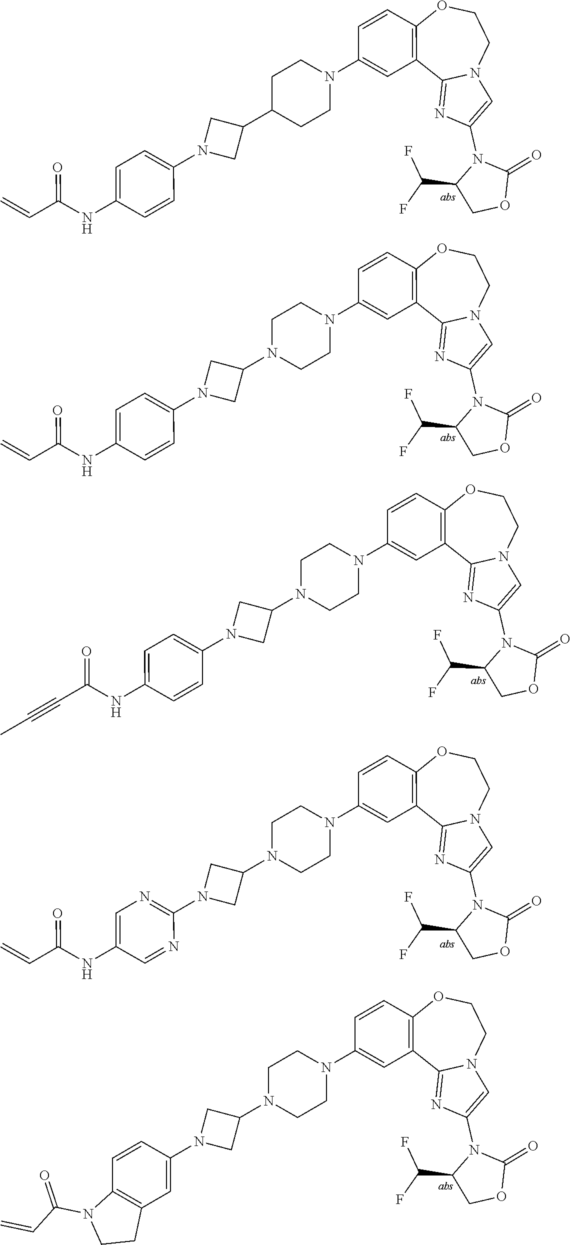

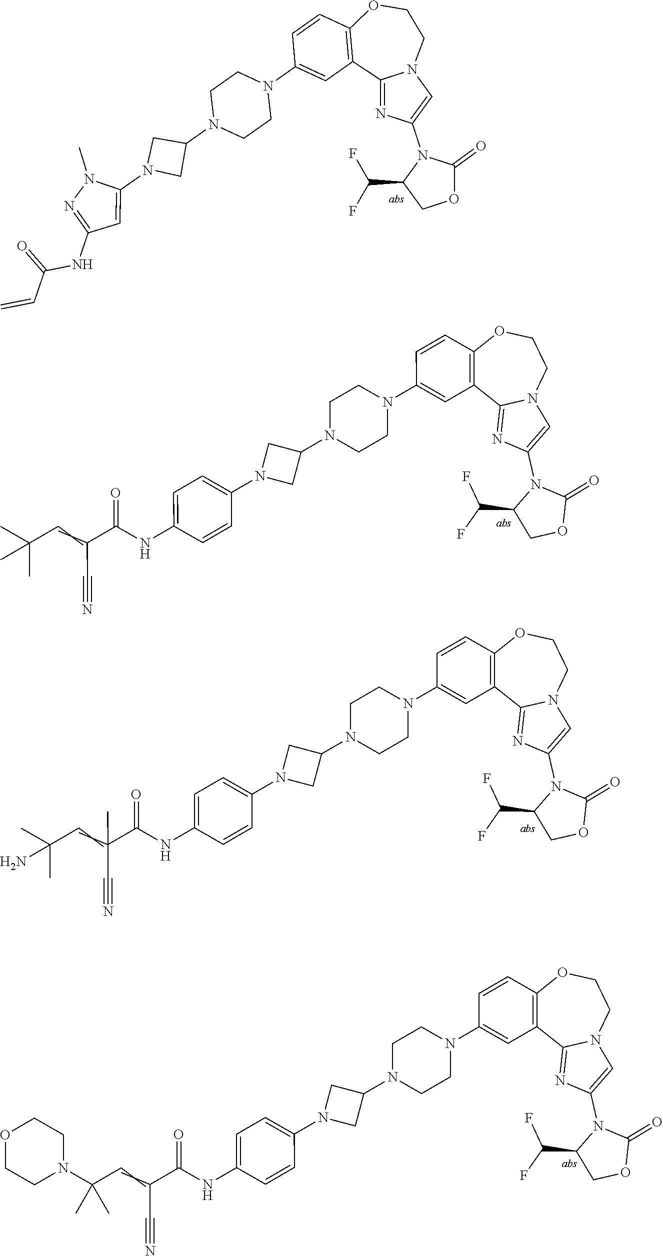

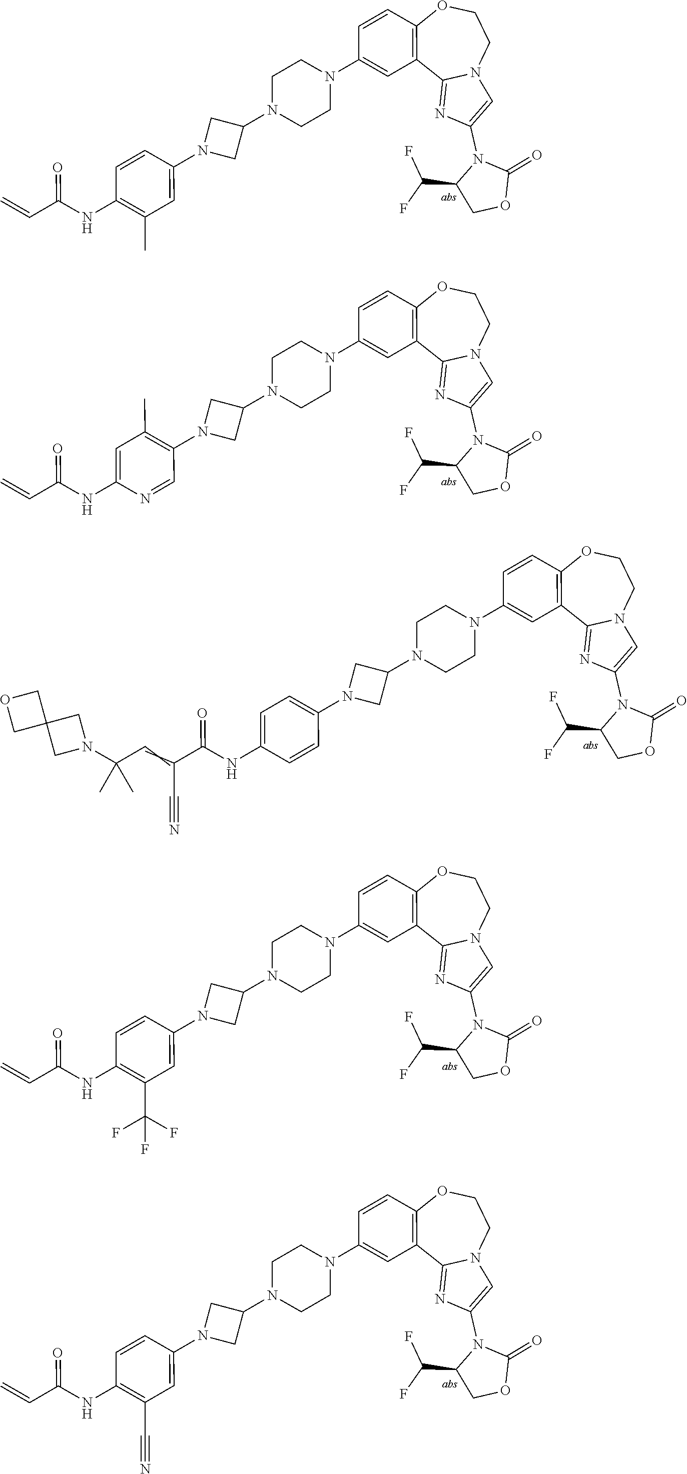 [SMILES: C=CC(=O)Nc1ccc(N2CC(N3CCN(c4ccc5c(c4)-c4nc(N6C(=O)OC[C@H]6C(F)F)cn4CCO5)CC3)C2)cc1C ; C=CC(=O)Nc1cc(C)c(N2CC(N3CCN(c4ccc5c(c4)-c4nc(N6C(=O)OC[C@H]6C(F)F)cn4CCO5)CC3)C2)cn1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)N1CC2(COC2)C1 ; C=CC(=O)Nc1ccc(N2CC(N3CCN(c4ccc5c(c4)-c4nc(N6C(=O)OC[C@H]6C(F)F)cn4CCO5)CC3)C2)cc1C(F)(F)F ; C=CC(=O)Nc1ccc(N2CC(N3CCN(c4ccc5c(c4)-c4nc(N6C(=O)OC[C@H]6C(F)F)cn4CCO5)CC3)C2)cc1C#N]

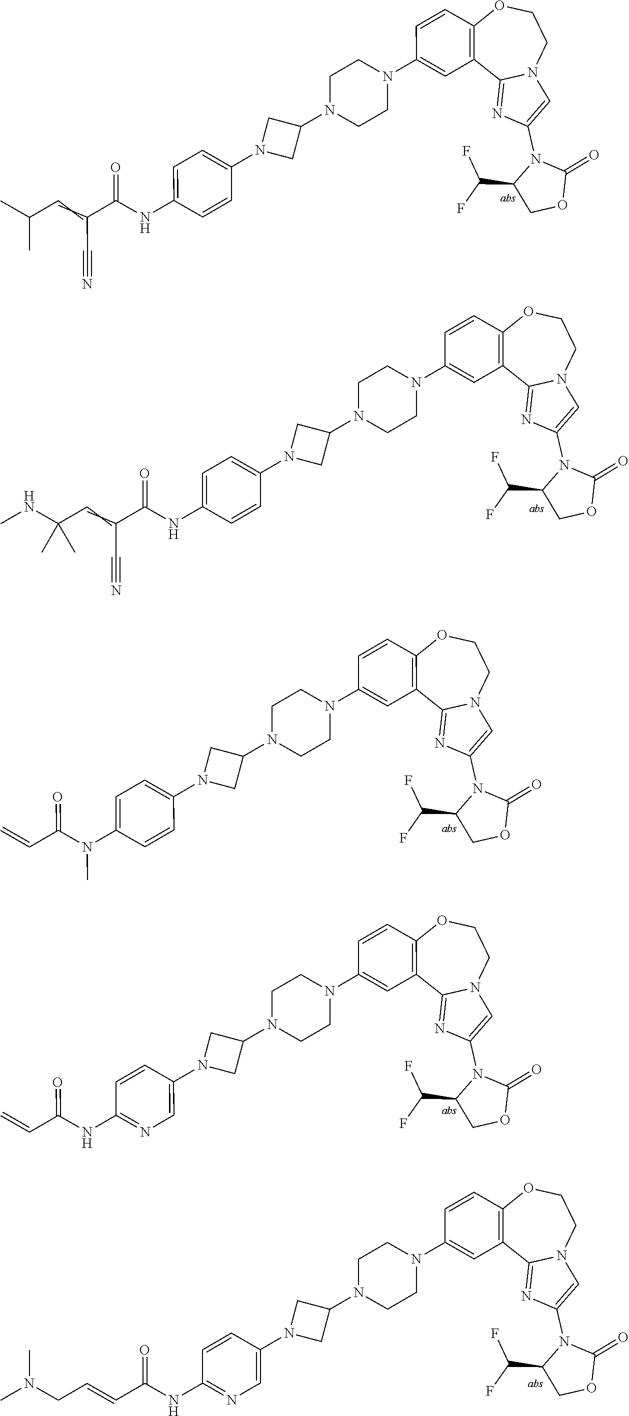 [SMILES: CC(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1 ; CNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1 ; C=CC(=O)N(C)c1ccc(N2CC(N3CCN(c4ccc5c(c4)-c4nc(N6C(=O)OC[C@H]6C(F)F)cn4CCO5)CC3)C2)cc1 ; C=CC(=O)Nc1ccc(N2CC(N3CCN(c4ccc5c(c4)-c4nc(N6C(=O)OC[C@H]6C(F)F)cn4CCO5)CC3)C2)cn1 ; CN(C)C/C=C/C(=O)Nc1ccc(N2CC(N3CCN(c4ccc5c(c4)-c4nc(N6C(=O)OC[C@H]6C(F)F)cn4CCO5)CC3)C2)cn1]

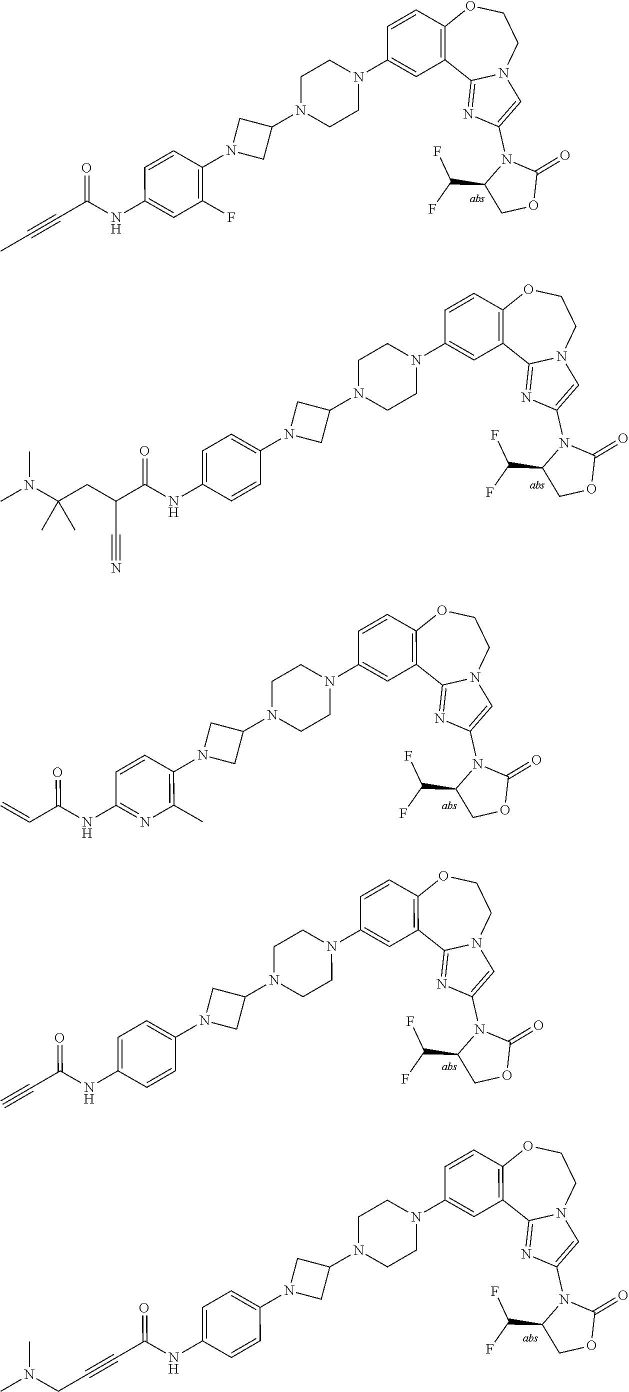 [SMILES: CC#CC(=O)Nc1ccc(N2CC(N3CCN(c4ccc5c(c4)-c4nc(N6C(=O)OC[C@H]6C(F)F)cn4CCO5)CC3)C2)c(F)c1 ; CN(C)C(C)(C)CC(C#N)C(=O)Nc1ccc(N2CC(N3CCN(c4ccc5c(c4)-c4nc(N6C(=O)OC[C@H]6C(F)F)cn4CCO5)CC3)C2)cc1 ; C=CC(=O)Nc1ccc(N2CC(N3CCN(c4ccc5c(c4)-c4nc(N6C(=O)OC[C@H]6C(F)F)cn4CCO5)CC3)C2)c(C)n1 ; C#CC(=O)Nc1ccc(N2CC(N3CCN(c4ccc5c(c4)-c4nc(N6C(=O)OC[C@H]6C(F)F)cn4CCO5)CC3)C2)cc1 ; CN(C)CC#CC(=O)Nc1ccc(N2CC(N3CCN(c4ccc5c(c4)-c4nc(N6C(=O)OC[C@H]6C(F)F)cn4CCO5)CC3)C2)cc1]

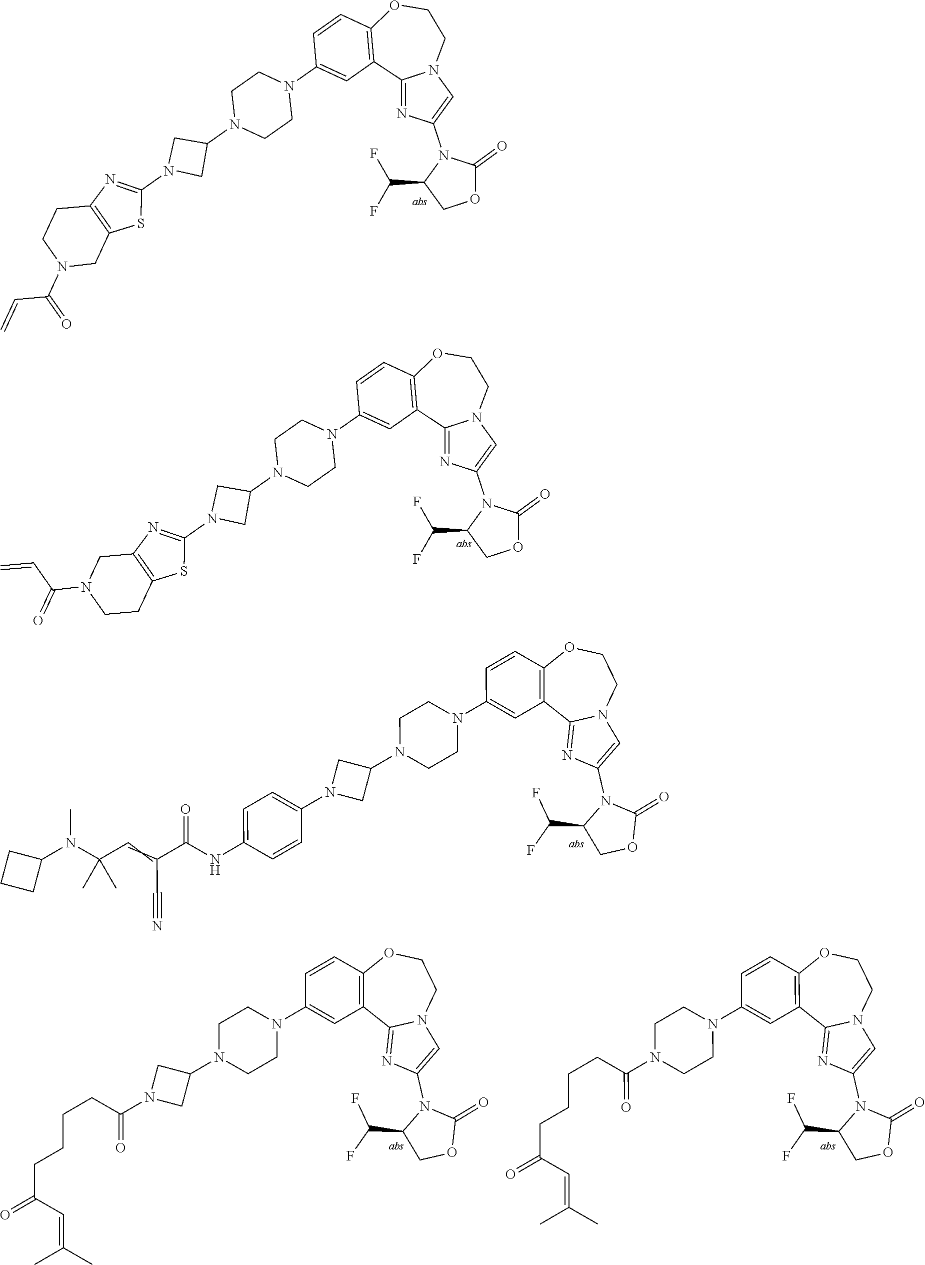 [SMILES: C=CC(=O)N1CCc2nc(N3CC(N4CCN(c5ccc6c(c5)-c5nc(N7C(=O)OC[C@H]7C(F)F)cn5CCO6)CC4)C3)sc2C1 ; C=CC(=O)N1CCc2sc(N3CC(N4CCN(c5ccc6c(c5)-c5nc(N7C(=O)OC[C@H]7C(F)F)cn5CCO6)CC4)C3)nc2C1 ; CN(C1CCC1)C(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1 ; CC(C)=CC(=O)CCCCC(=O)N1CC(N2CCN(c3ccc4c(c3)-c3nc(N5C(=O)OC[C@H]5C(F)F)cn3CCO4)CC2)C1 ; CC(C)=CC(=O)CCCCC(=O)N1CCN(c2ccc3c(c2)-c2nc(N4C(=O)OC[C@H]4C(F)F)cn2CCO3)CC1]

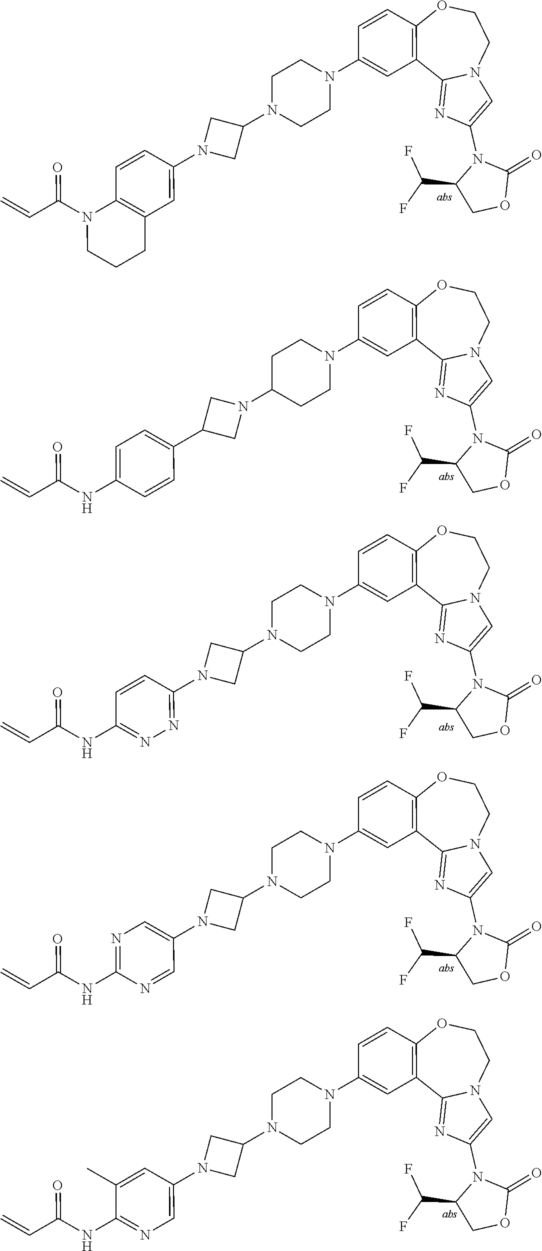

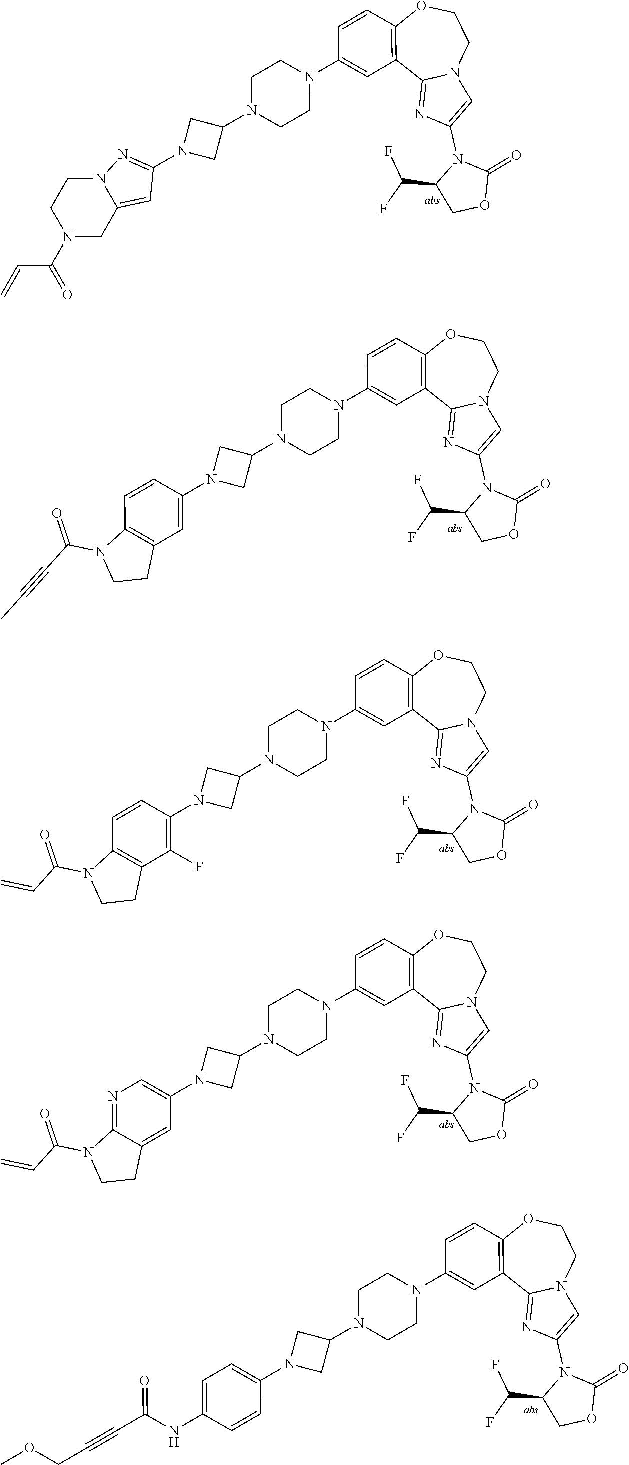 [SMILES: C=CC(=O)N1CCn2nc(N3CC(N4CCN(c5ccc6c(c5)-c5nc(N7C(=O)OC[C@H]7C(F)F)cn5CCO6)CC4)C3)cc2C1 ; CC#CC(=O)N1CCc2cc(N3CC(N4CCN(c5ccc6c(c5)-c5nc(N7C(=O)OC[C@H]7C(F)F)cn5CCO6)CC4)C3)ccc21 ; C=CC(=O)N1CCc2c1ccc(N1CC(N3CCN(c4ccc5c(c4)-c4nc(N6C(=O)OC[C@H]6C(F)F)cn4CCO5)CC3)C1)c2F ; C=CC(=O)N1CCc2cc(N3CC(N4CCN(c5ccc6c(c5)-c5nc(N7C(=O)OC[C@H]7C(F)F)cn5CCO6)CC4)C3)cnc21 ; COCC#CC(=O)Nc1ccc(N2CC(N3CCN(c4ccc5c(c4)-c4nc(N6C(=O)OC[C@H]6C(F)F)cn4CCO5)CC3)C2)cc1]

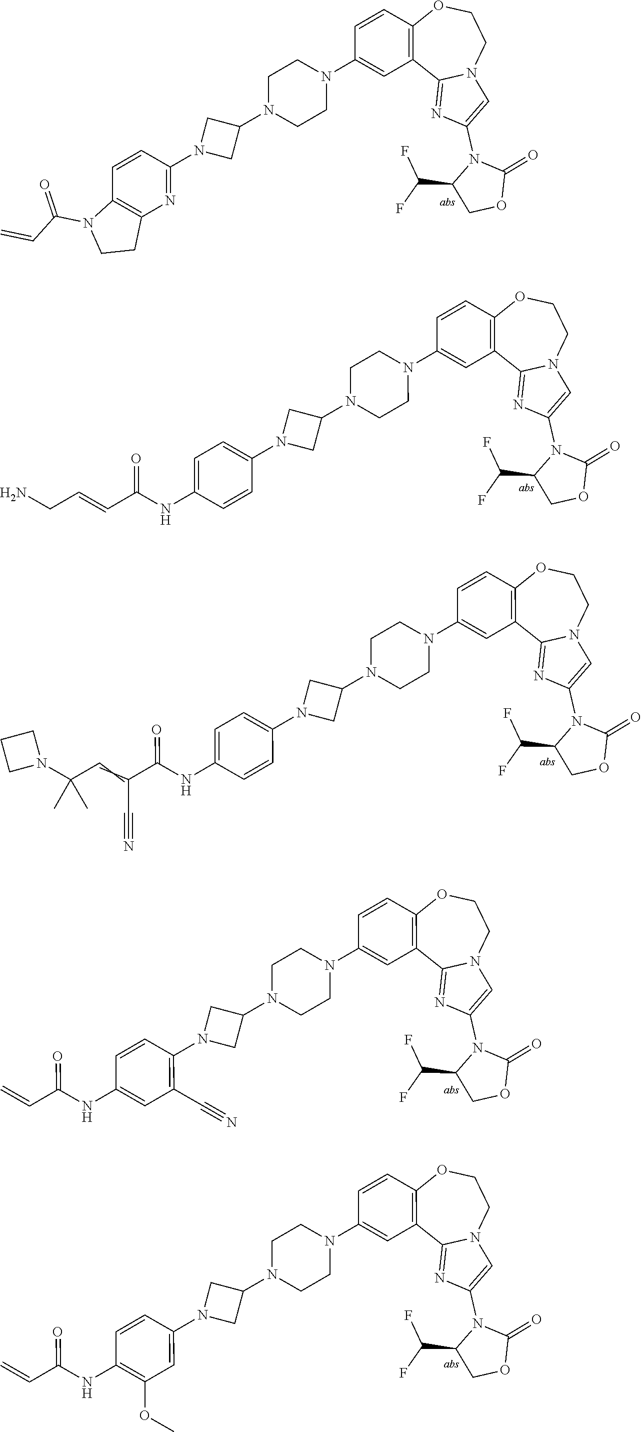 [SMILES: C=CC(=O)N1CCc2nc(N3CC(N4CCN(c5ccc6c(c5)-c5nc(N7C(=O)OC[C@H]7C(F)F)cn5CCO6)CC4)C3)ccc21 ; NC/C=C/C(=O)Nc1ccc(N2CC(N3CCN(c4ccc5c(c4)-c4nc(N6C(=O)OC[C@H]6C(F)F)cn4CCO5)CC3)C2)cc1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)N1CCC1 ; C=CC(=O)Nc1ccc(N2CC(N3CCN(c4ccc5c(c4)-c4nc(N6C(=O)OC[C@H]6C(F)F)cn4CCO5)CC3)C2)c(C#N)c1 ; C=CC(=O)Nc1ccc(N2CC(N3CCN(c4ccc5c(c4)-c4nc(N6C(=O)OC[C@H]6C(F)F)cn4CCO5)CC3)C2)cc1OC]

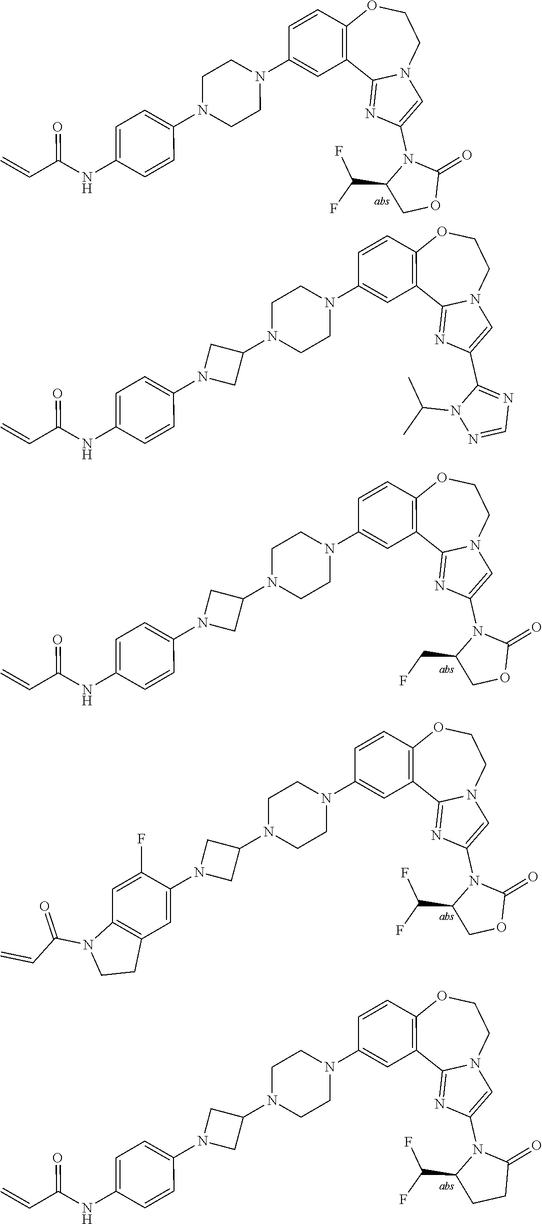

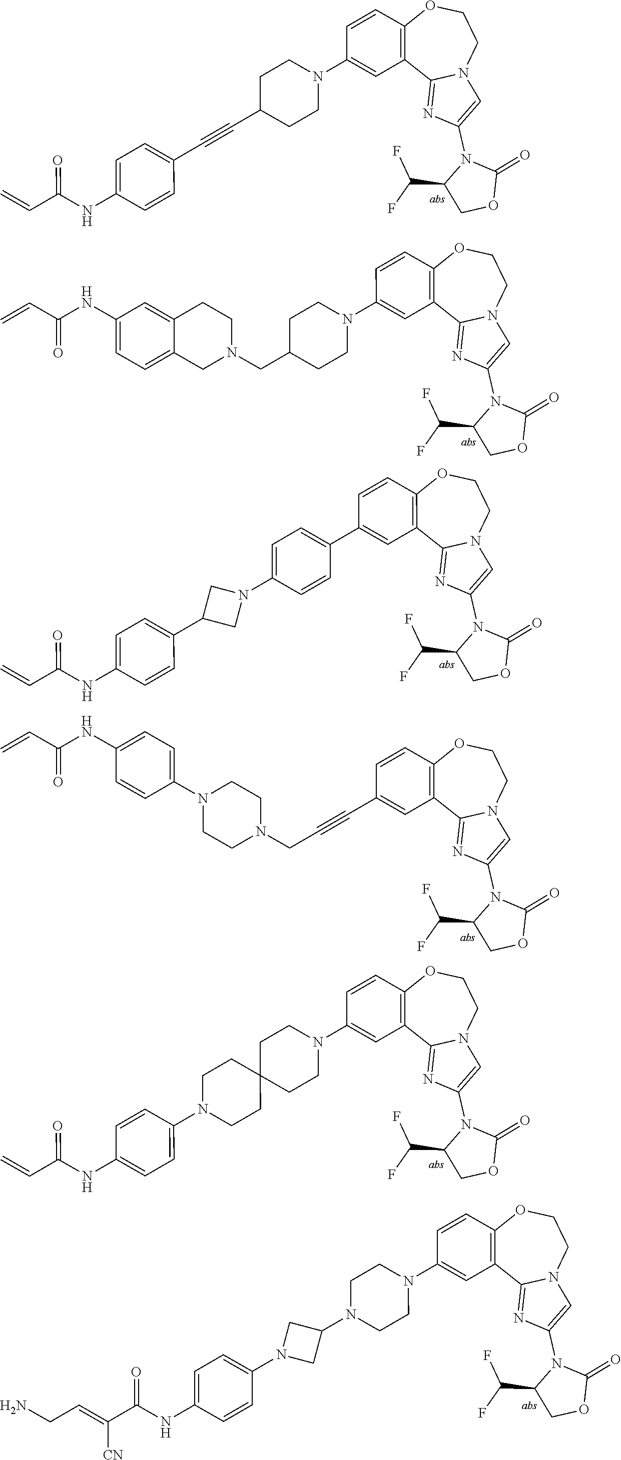

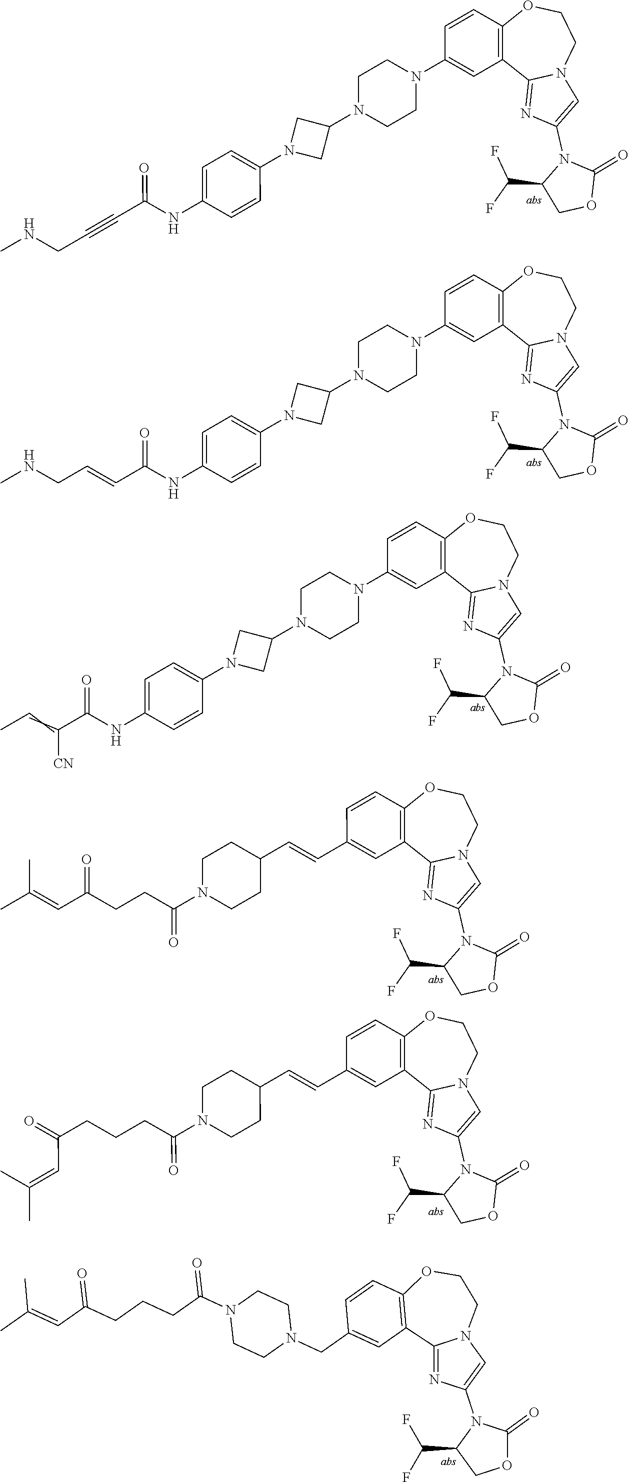

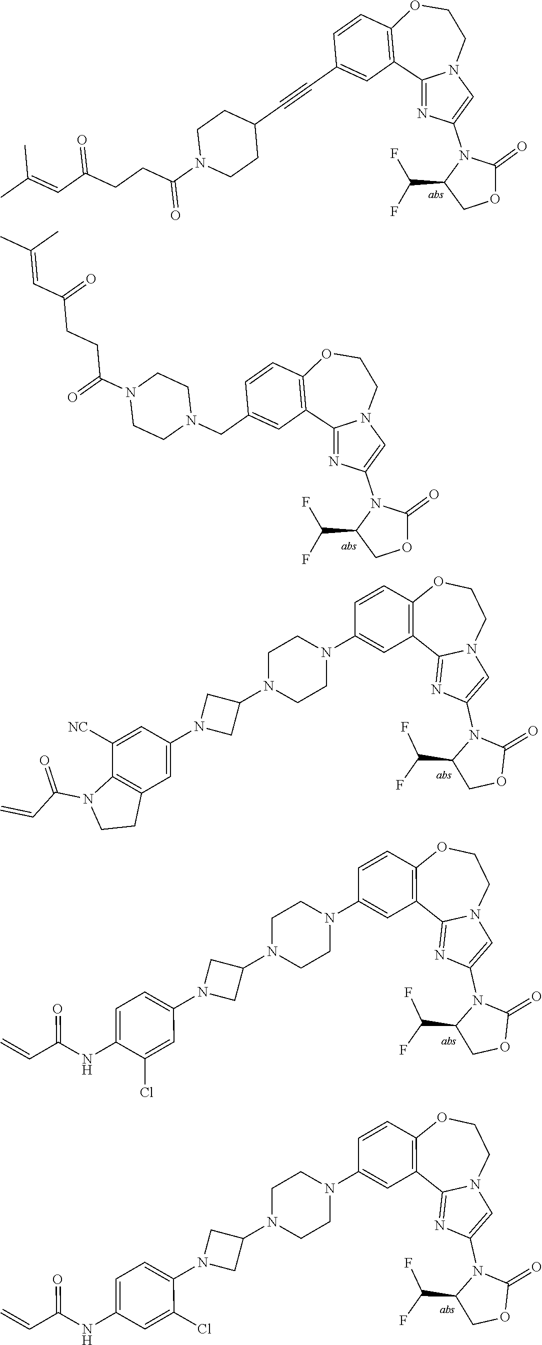

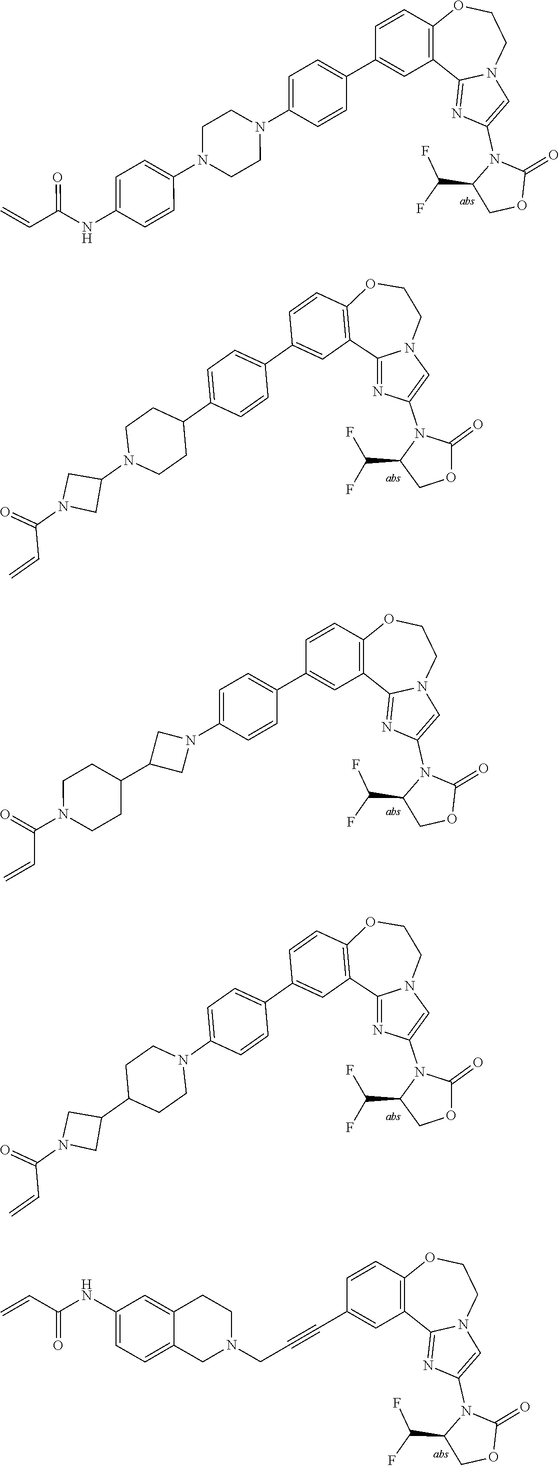 [SMILES: C=CC(=O)Nc1ccc(N2CCN(c3ccc(-c4ccc5c(c4)-c4nc(N6C(=O)OC[C@H]6C(F)F)cn4CCO5)cc3)CC2)cc1 ; C=CC(=O)N1CC(N2CCC(c3ccc(-c4ccc5c(c4)-c4nc(N6C(=O)OC[C@H]6C(F)F)cn4CCO5)cc3)CC2)C1 ; C=CC(=O)N1CCC(C2CN(c3ccc(-c4ccc5c(c4)-c4nc(N6C(=O)OC[C@H]6C(F)F)cn4CCO5)cc3)C2)CC1 ; C=CC(=O)N1CC(C2CCN(c3ccc(-c4ccc5c(c4)-c4nc(N6C(=O)OC[C@H]6C(F)F)cn4CCO5)cc3)CC2)C1 ; C=CC(=O)Nc1ccc2c(c1)CCN(CC#Cc1ccc3c(c1)-c1nc(N4C(=O)OC[C@H]4C(F)F)cn1CCO3)C2]

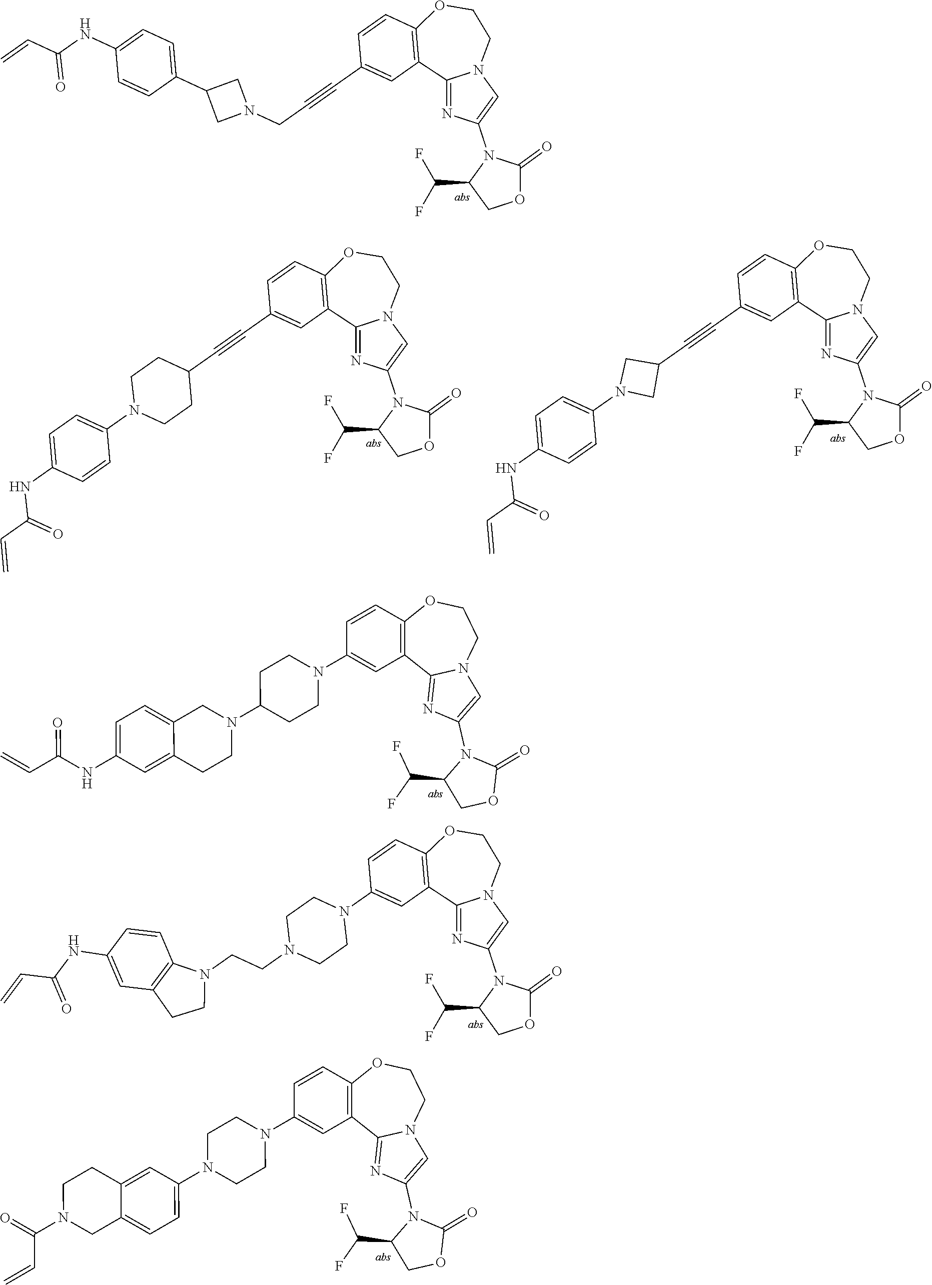

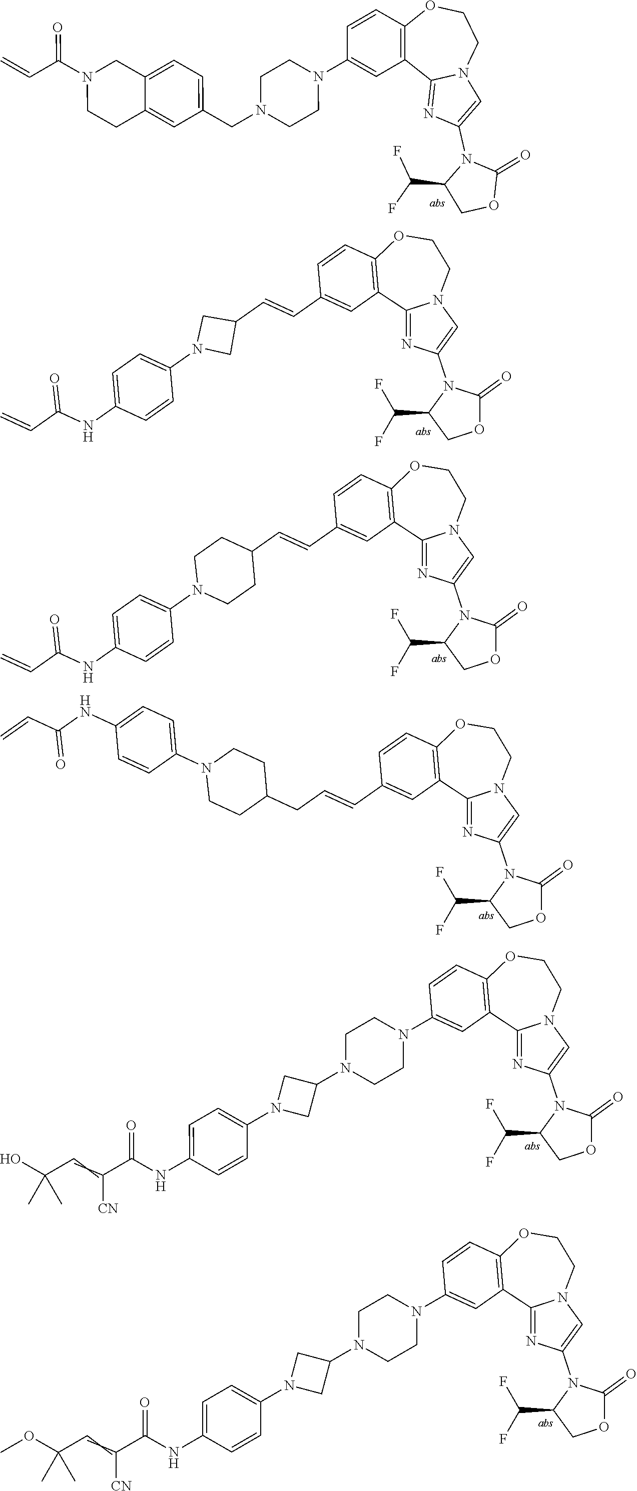

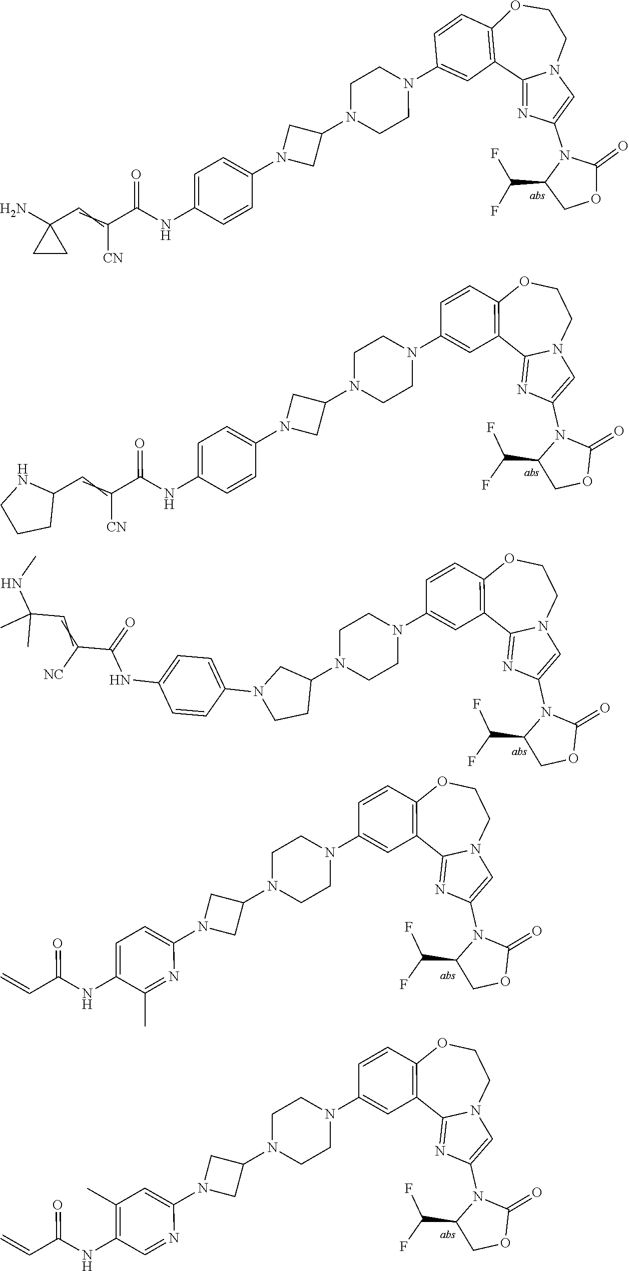

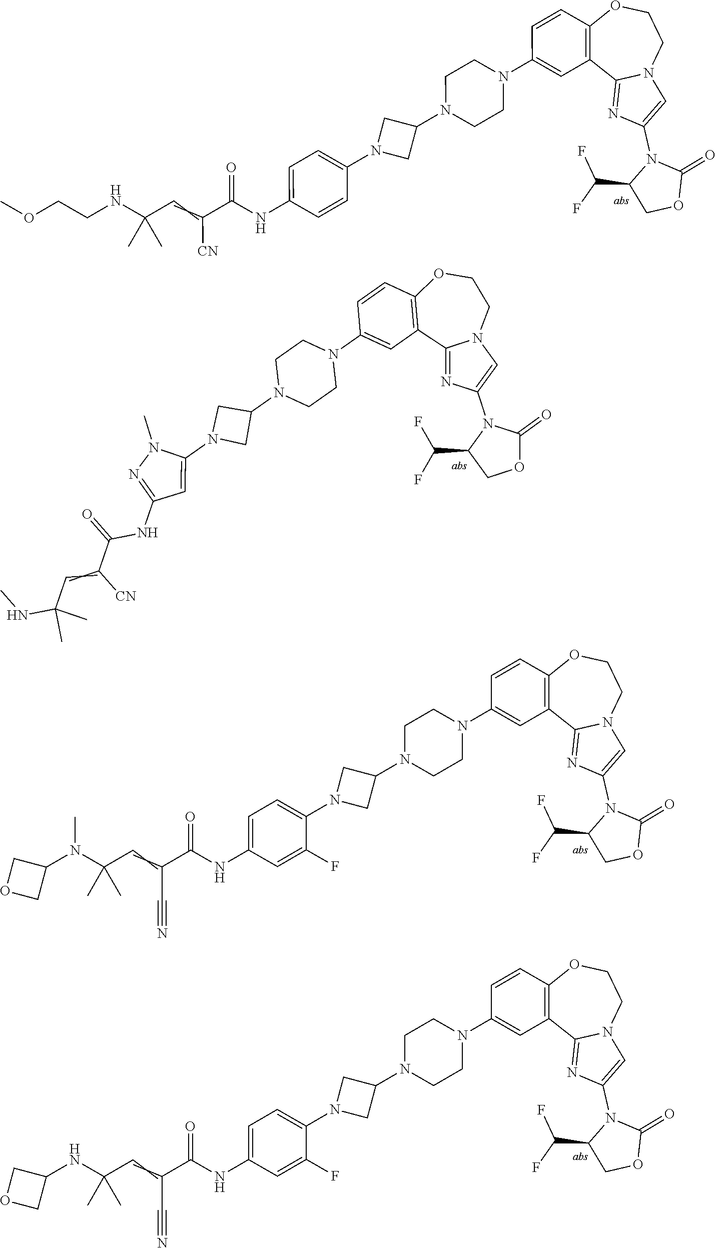 [SMILES: COCCNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1 ; CNC(C)(C)C=C(C#N)C(=O)Nc1cc(N2CC(N3CCN(c4ccc5c(c4)-c4nc(N6C(=O)OC[C@H]6C(F)F)cn4CCO5)CC3)C2)n(C)n1 ; CN(C1COC1)C(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1)NC1COC1]

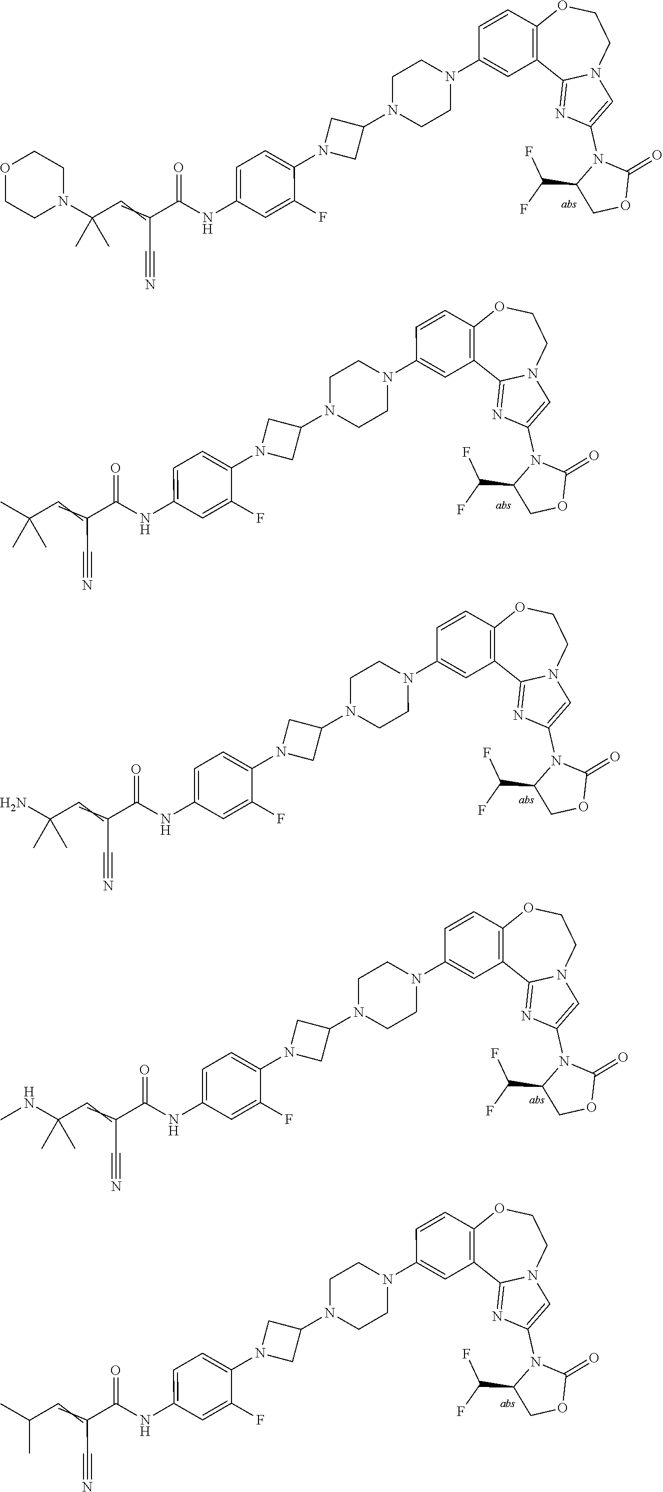

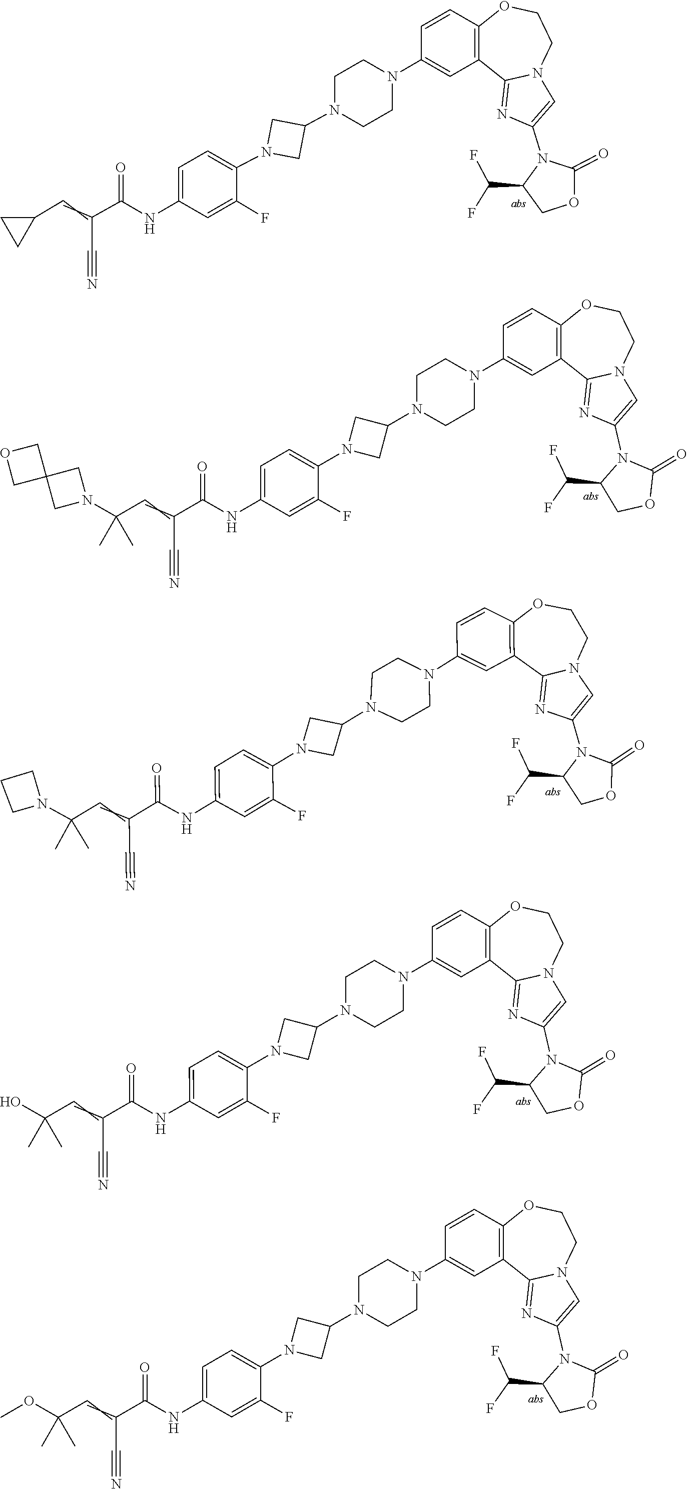

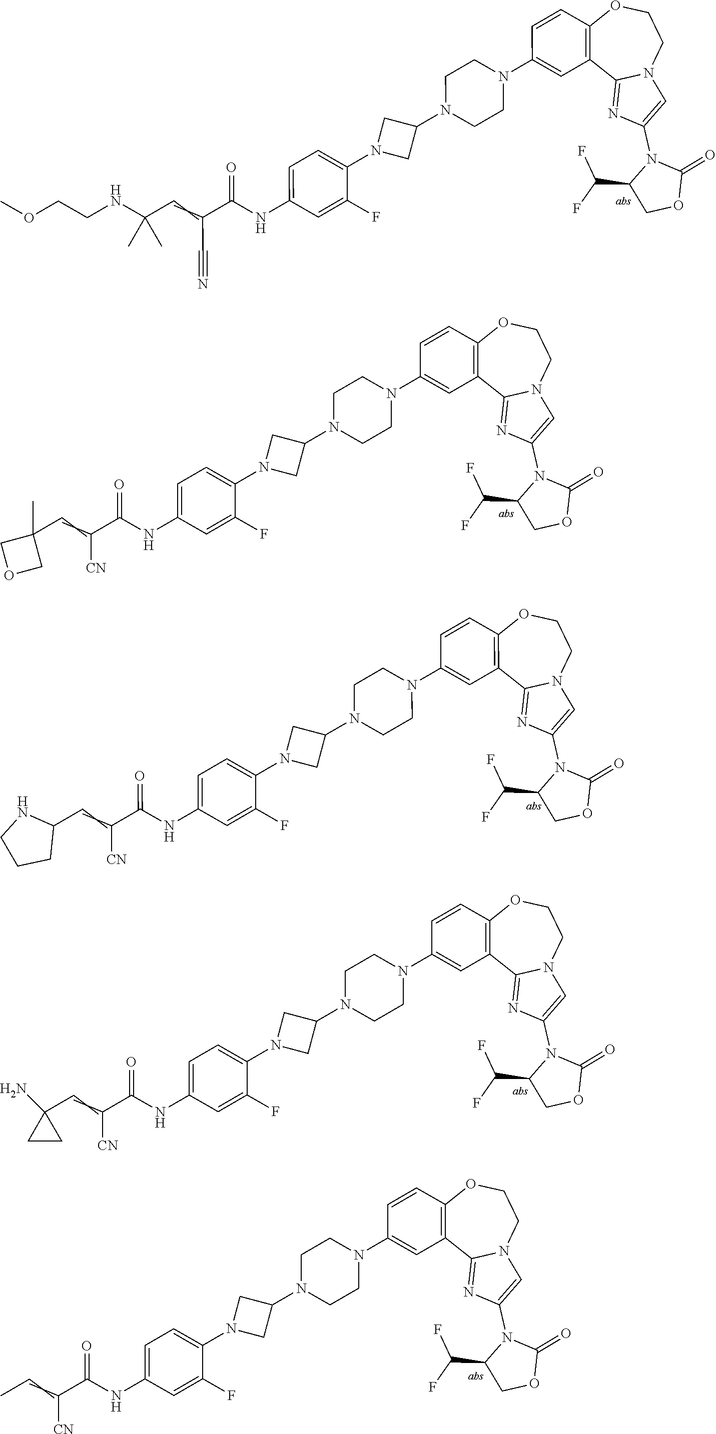 [SMILES: COCCNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1 ; CC1(C=C(C#N)C(=O)Nc2ccc(N3CC(N4CCN(c5ccc6c(c5)-c5nc(N7C(=O)OC[C@H]7C(F)F)cn5CCO6)CC4)C3)c(F)c2)COC1 ; N#CC(=CC1CCCN1)C(=O)Nc1ccc(N2CC(N3CCN(c4ccc5c(c4)-c4nc(N6C(=O)OC[C@H]6C(F)F)cn4CCO5)CC3)C2)c(F)c1 ; N#CC(=CC1(N)CC1)C(=O)Nc1ccc(N2CC(N3CCN(c4ccc5c(c4)-c4nc(N6C(=O)OC[C@H]6C(F)F)cn4CCO5)CC3)C2)c(F)c1 ; CC=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1]

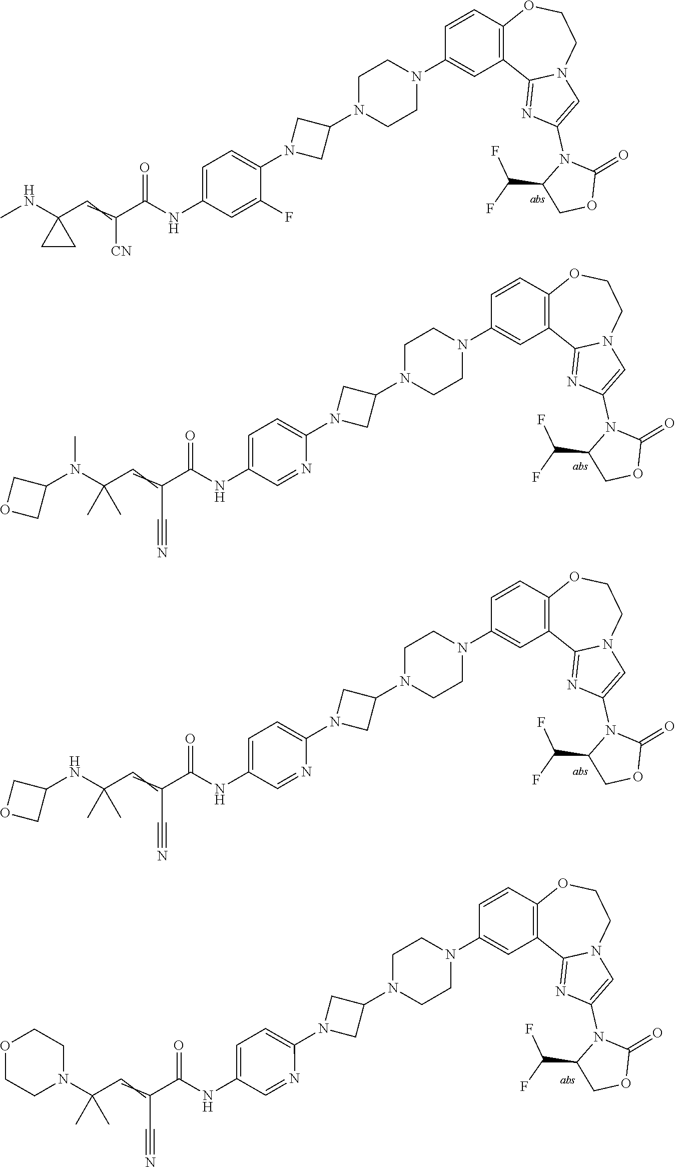 [SMILES: CNC1(C=C(C#N)C(=O)Nc2ccc(N3CC(N4CCN(c5ccc6c(c5)-c5nc(N7C(=O)OC[C@H]7C(F)F)cn5CCO6)CC4)C3)c(F)c2)CC1 ; CN(C1COC1)C(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)nc1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)nc1)NC1COC1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)nc1)N1CCOCC1]

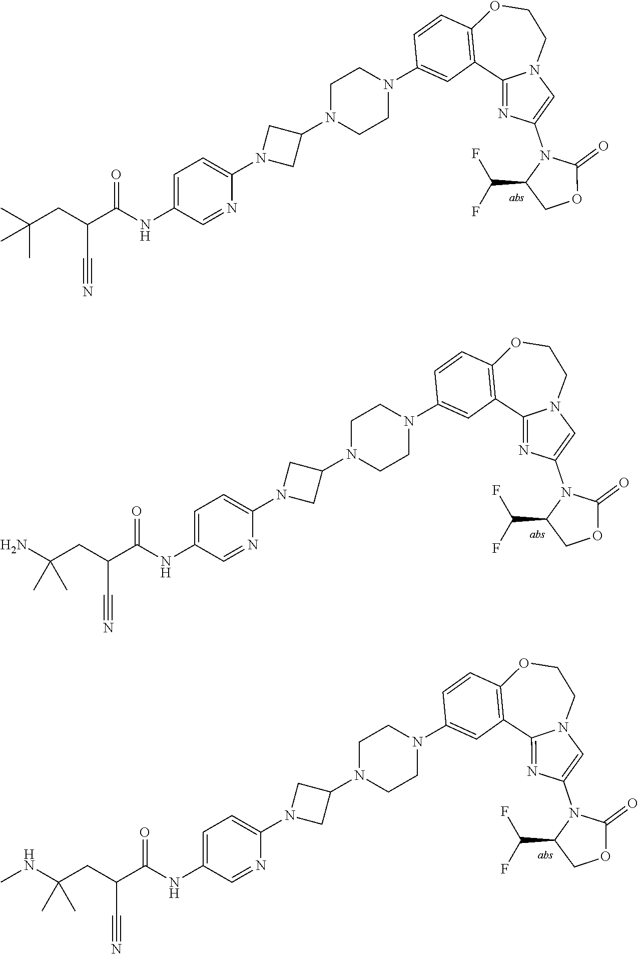 [SMILES: CC(C)(C)CC(C#N)C(=O)Nc1ccc(N2CC(N3CCN(c4ccc5c(c4)-c4nc(N6C(=O)OC[C@H]6C(F)F)cn4CCO5)CC3)C2)nc1 ; CC(C)(N)CC(C#N)C(=O)Nc1ccc(N2CC(N3CCN(c4ccc5c(c4)-c4nc(N6C(=O)OC[C@H]6C(F)F)cn4CCO5)CC3)C2)nc1 ; CNC(C)(C)CC(C#N)C(=O)Nc1ccc(N2CC(N3CCN(c4ccc5c(c4)-c4nc(N6C(=O)OC[C@H]6C(F)F)cn4CCO5)CC3)C2)nc1]

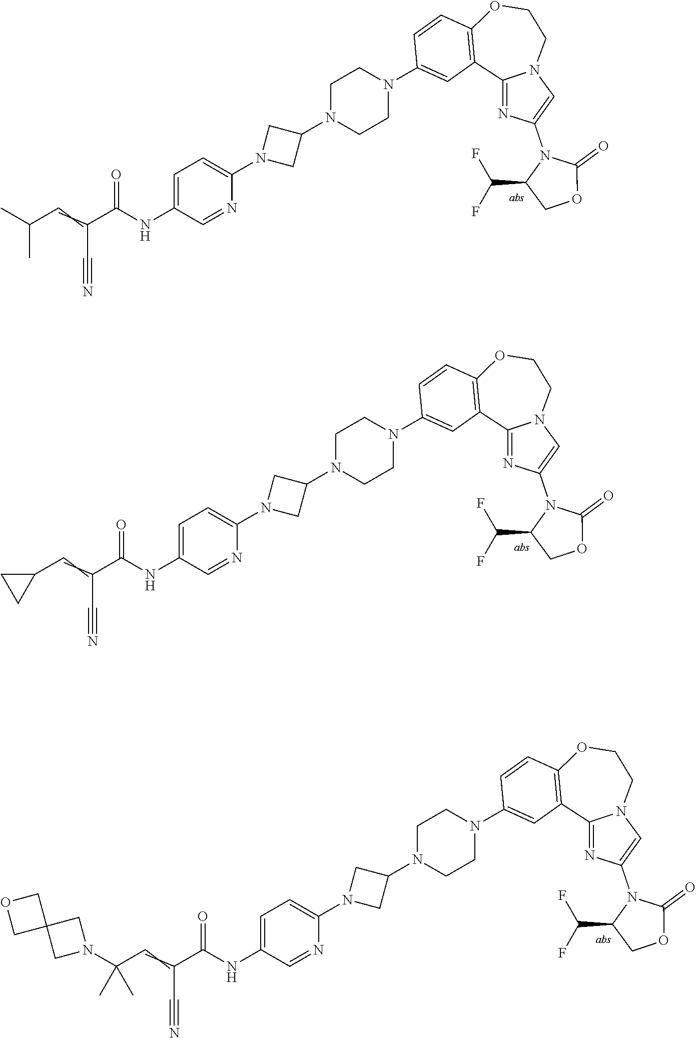 [SMILES: CC(C)C=C(C#N)C(=O)Nc1ccc(N2CC(N3CCN(c4ccc5c(c4)-c4nc(N6C(=O)OC[C@H]6C(F)F)cn4CCO5)CC3)C2)nc1 ; N#CC(=CC1CC1)C(=O)Nc1ccc(N2CC(N3CCN(c4ccc5c(c4)-c4nc(N6C(=O)OC[C@H]6C(F)F)cn4CCO5)CC3)C2)nc1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)nc1)N1CC2(COC2)C1]

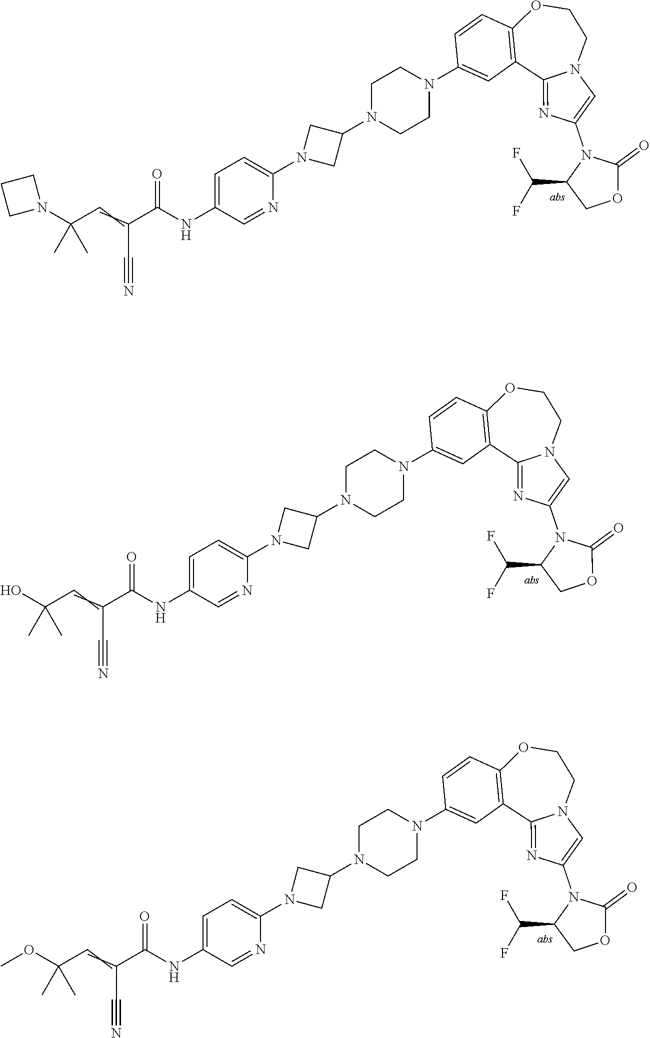 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)nc1)N1CCC1 ; CC(C)(O)C=C(C#N)C(=O)Nc1ccc(N2CC(N3CCN(c4ccc5c(c4)-c4nc(N6C(=O)OC[C@H]6C(F)F)cn4CCO5)CC3)C2)nc1 ; COC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)nc1]

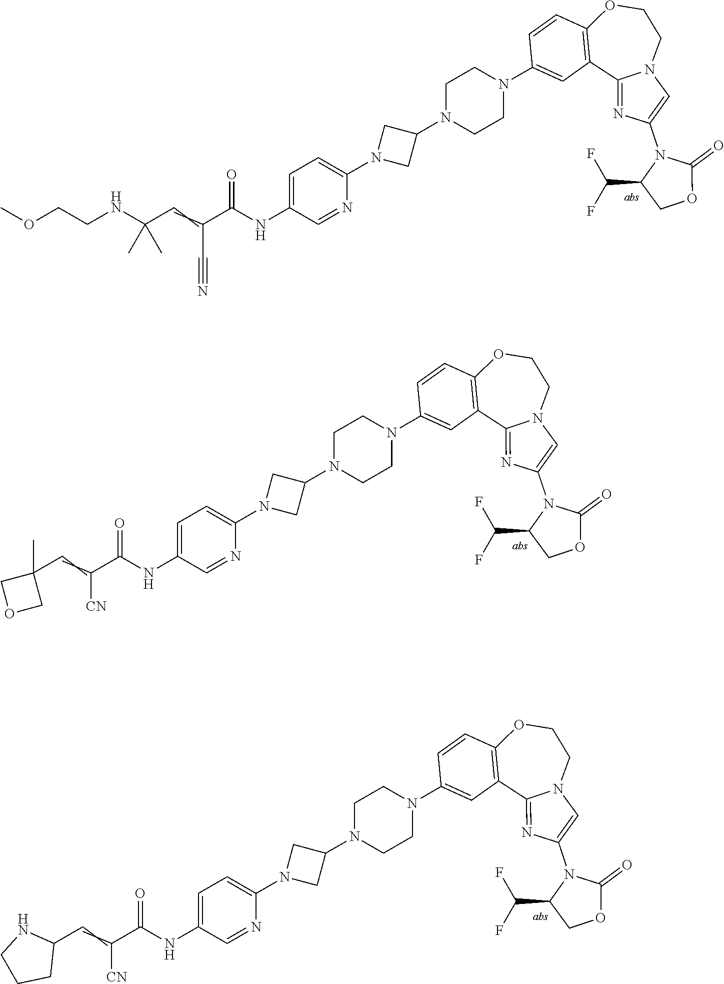 [SMILES: COCCNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)nc1 ; CC1(C=C(C#N)C(=O)Nc2ccc(N3CC(N4CCN(c5ccc6c(c5)-c5nc(N7C(=O)OC[C@H]7C(F)F)cn5CCO6)CC4)C3)nc2)COC1 ; N#CC(=CC1CCCN1)C(=O)Nc1ccc(N2CC(N3CCN(c4ccc5c(c4)-c4nc(N6C(=O)OC[C@H]6C(F)F)cn4CCO5)CC3)C2)nc1]

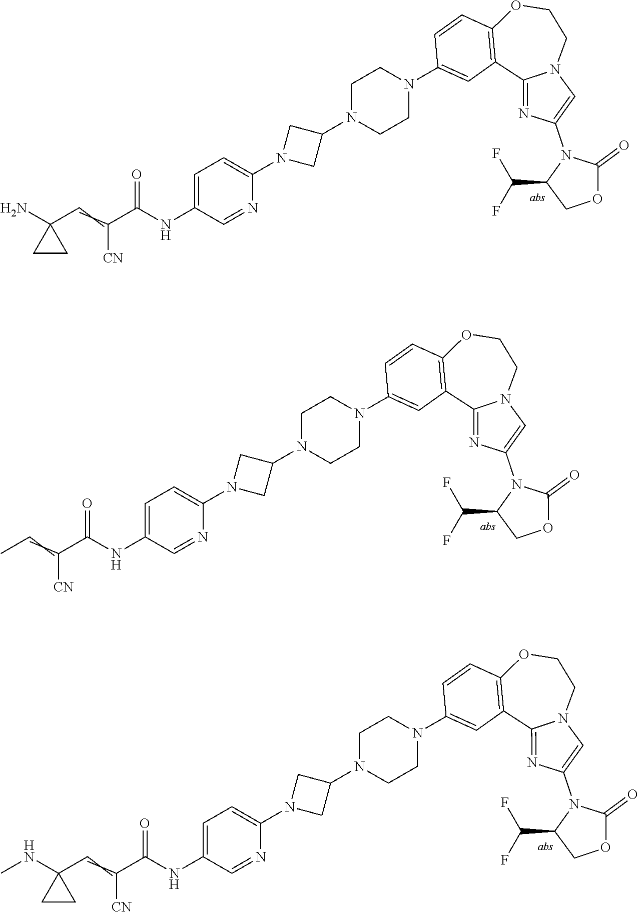 [SMILES: N#CC(=CC1(N)CC1)C(=O)Nc1ccc(N2CC(N3CCN(c4ccc5c(c4)-c4nc(N6C(=O)OC[C@H]6C(F)F)cn4CCO5)CC3)C2)nc1 ; CC=C(C#N)C(=O)Nc1ccc(N2CC(N3CCN(c4ccc5c(c4)-c4nc(N6C(=O)OC[C@H]6C(F)F)cn4CCO5)CC3)C2)nc1 ; CNC1(C=C(C#N)C(=O)Nc2ccc(N3CC(N4CCN(c5ccc6c(c5)-c5nc(N7C(=O)OC[C@H]7C(F)F)cn5CCO6)CC4)C3)nc2)CC1]

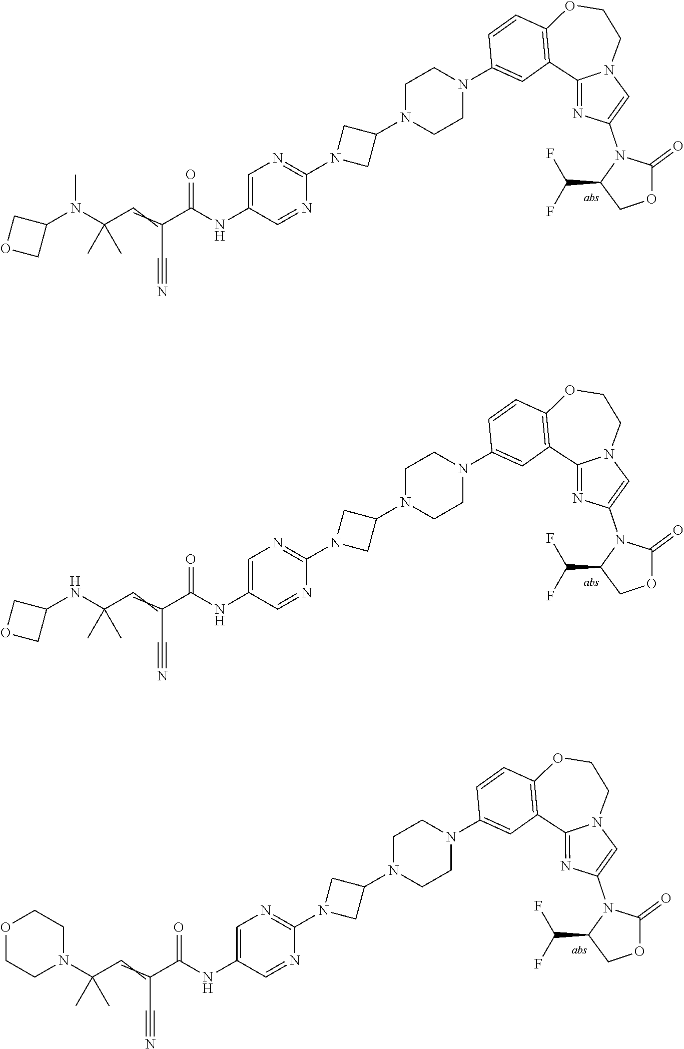 [SMILES: CN(C1COC1)C(C)(C)C=C(C#N)C(=O)Nc1cnc(N2CC(N3CCN(c4ccc5c(c4)-c4nc(N6C(=O)OC[C@H]6C(F)F)cn4CCO5)CC3)C2)nc1 ; CC(C)(C=C(C#N)C(=O)Nc1cnc(N2CC(N3CCN(c4ccc5c(c4)-c4nc(N6C(=O)OC[C@H]6C(F)F)cn4CCO5)CC3)C2)nc1)NC1COC1 ; CC(C)(C=C(C#N)C(=O)Nc1cnc(N2CC(N3CCN(c4ccc5c(c4)-c4nc(N6C(=O)OC[C@H]6C(F)F)cn4CCO5)CC3)C2)nc1)N1CCOCC1]

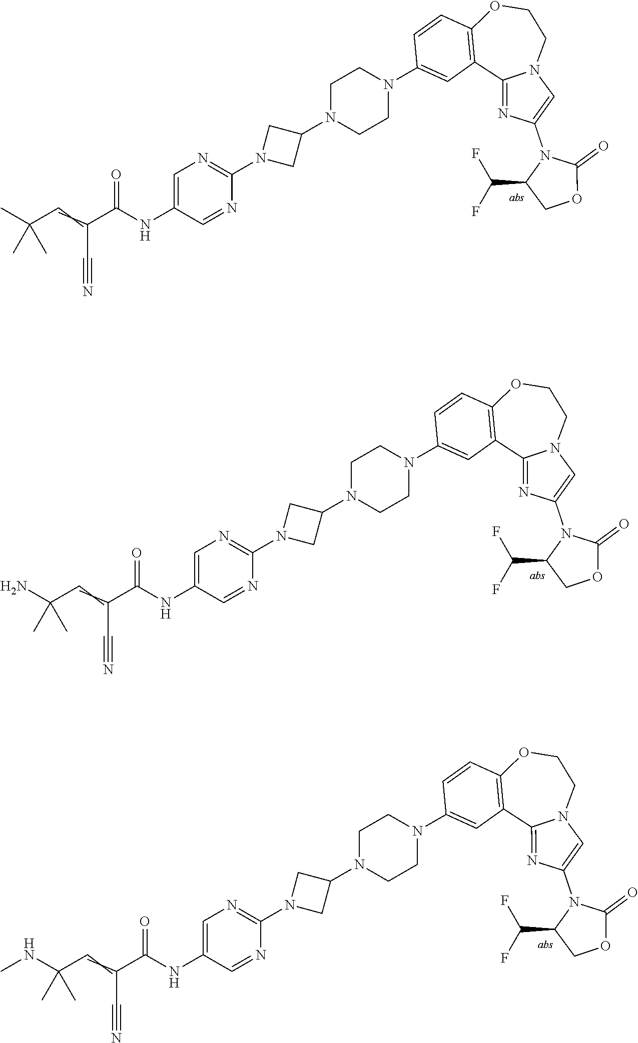 [SMILES: CC(C)(C)C=C(C#N)C(=O)Nc1cnc(N2CC(N3CCN(c4ccc5c(c4)-c4nc(N6C(=O)OC[C@H]6C(F)F)cn4CCO5)CC3)C2)nc1 ; CC(C)(N)C=C(C#N)C(=O)Nc1cnc(N2CC(N3CCN(c4ccc5c(c4)-c4nc(N6C(=O)OC[C@H]6C(F)F)cn4CCO5)CC3)C2)nc1 ; CNC(C)(C)C=C(C#N)C(=O)Nc1cnc(N2CC(N3CCN(c4ccc5c(c4)-c4nc(N6C(=O)OC[C@H]6C(F)F)cn4CCO5)CC3)C2)nc1]

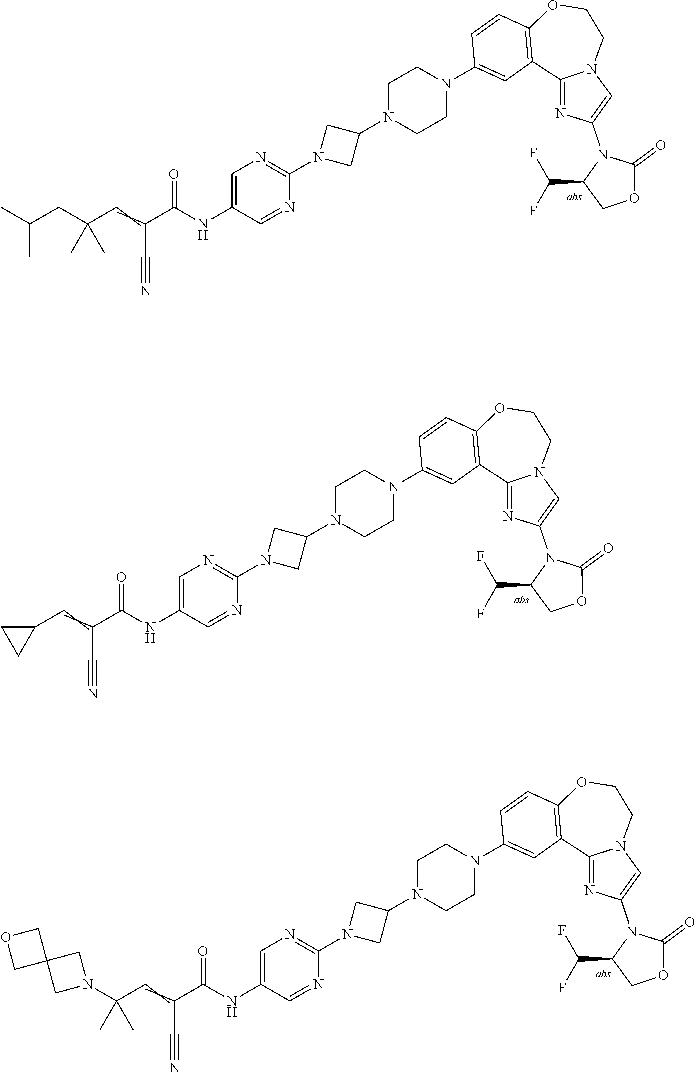 [SMILES: CC(C)CC(C)(C)C=C(C#N)C(=O)Nc1cnc(N2CC(N3CCN(c4ccc5c(c4)-c4nc(N6C(=O)OC[C@H]6C(F)F)cn4CCO5)CC3)C2)nc1 ; N#CC(=CC1CC1)C(=O)Nc1cnc(N2CC(N3CCN(c4ccc5c(c4)-c4nc(N6C(=O)OC[C@H]6C(F)F)cn4CCO5)CC3)C2)nc1 ; CC(C)(C=C(C#N)C(=O)Nc1cnc(N2CC(N3CCN(c4ccc5c(c4)-c4nc(N6C(=O)OC[C@H]6C(F)F)cn4CCO5)CC3)C2)nc1)N1CC2(COC2)C1]

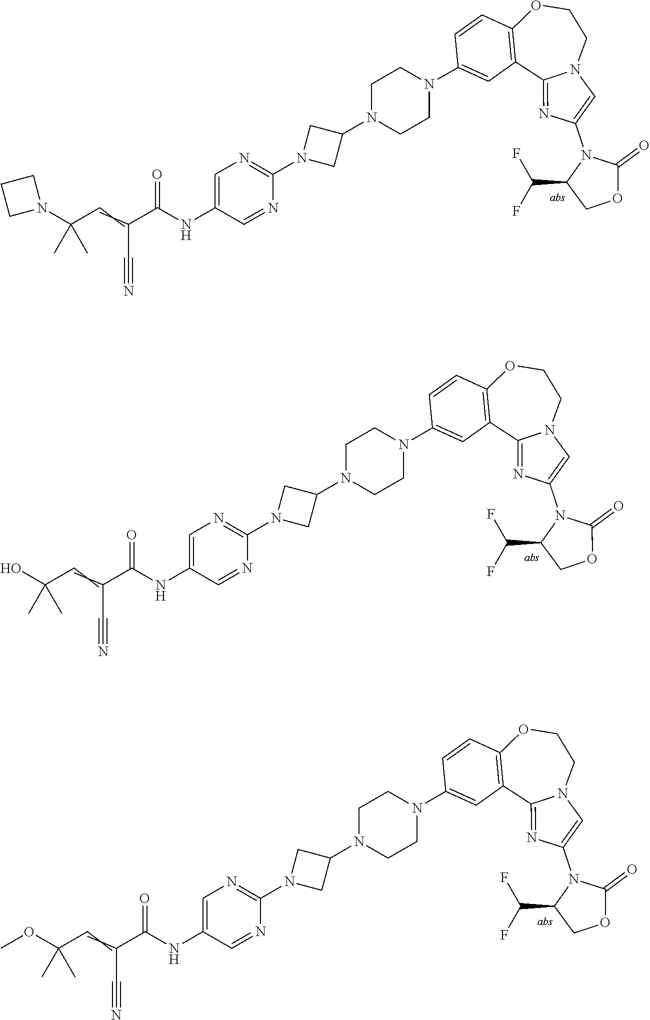 [SMILES: CC(C)(C=C(C#N)C(=O)Nc1cnc(N2CC(N3CCN(c4ccc5c(c4)-c4nc(N6C(=O)OC[C@H]6C(F)F)cn4CCO5)CC3)C2)nc1)N1CCC1 ; CC(C)(O)C=C(C#N)C(=O)Nc1cnc(N2CC(N3CCN(c4ccc5c(c4)-c4nc(N6C(=O)OC[C@H]6C(F)F)cn4CCO5)CC3)C2)nc1 ; COC(C)(C)C=C(C#N)C(=O)Nc1cnc(N2CC(N3CCN(c4ccc5c(c4)-c4nc(N6C(=O)OC[C@H]6C(F)F)cn4CCO5)CC3)C2)nc1]

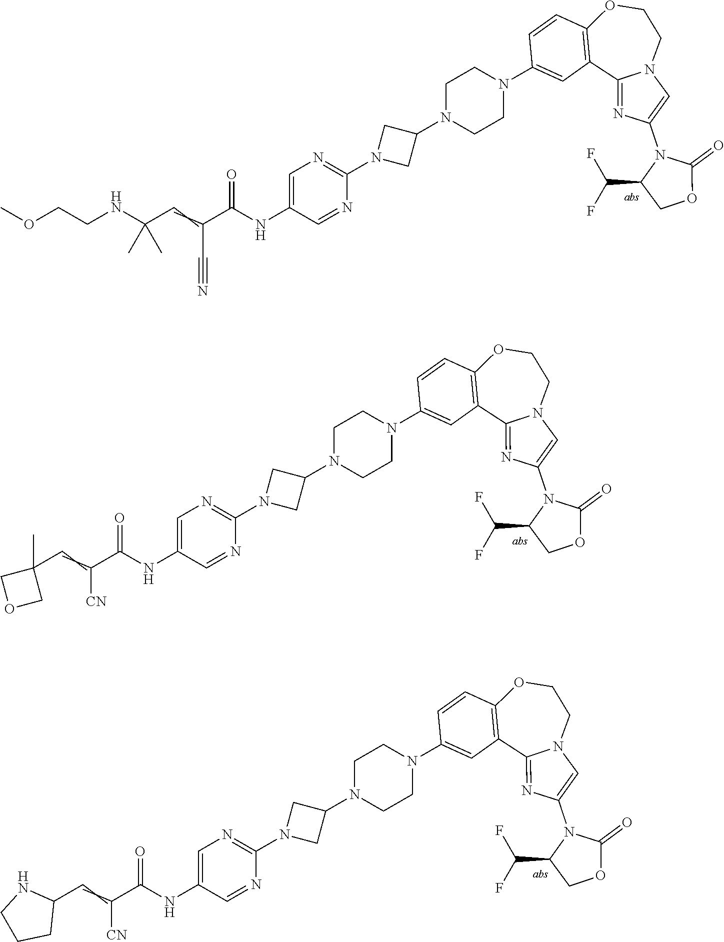 [SMILES: COCCNC(C)(C)C=C(C#N)C(=O)Nc1cnc(N2CC(N3CCN(c4ccc5c(c4)-c4nc(N6C(=O)OC[C@H]6C(F)F)cn4CCO5)CC3)C2)nc1 ; CC1(C=C(C#N)C(=O)Nc2cnc(N3CC(N4CCN(c5ccc6c(c5)-c5nc(N7C(=O)OC[C@H]7C(F)F)cn5CCO6)CC4)C3)nc2)COC1 ; N#CC(=CC1CCCN1)C(=O)Nc1cnc(N2CC(N3CCN(c4ccc5c(c4)-c4nc(N6C(=O)OC[C@H]6C(F)F)cn4CCO5)CC3)C2)nc1]

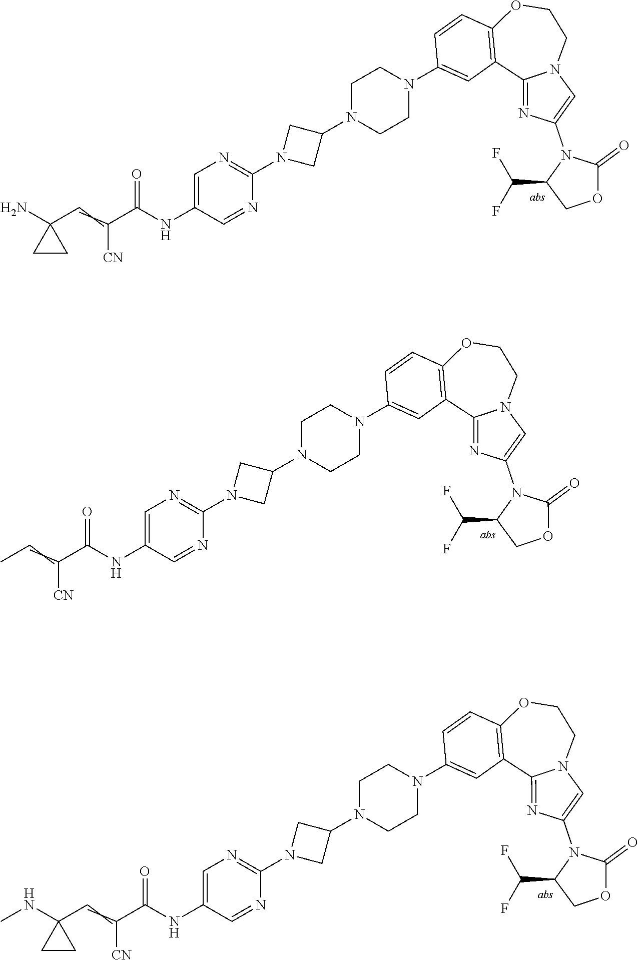 [SMILES: N#CC(=CC1(N)CC1)C(=O)Nc1cnc(N2CC(N3CCN(c4ccc5c(c4)-c4nc(N6C(=O)OC[C@H]6C(F)F)cn4CCO5)CC3)C2)nc1 ; CC=C(C#N)C(=O)Nc1cnc(N2CC(N3CCN(c4ccc5c(c4)-c4nc(N6C(=O)OC[C@H]6C(F)F)cn4CCO5)CC3)C2)nc1 ; CNC1(C=C(C#N)C(=O)Nc2cnc(N3CC(N4CCN(c5ccc6c(c5)-c5nc(N7C(=O)OC[C@H]7C(F)F)cn5CCO6)CC4)C3)nc2)CC1]

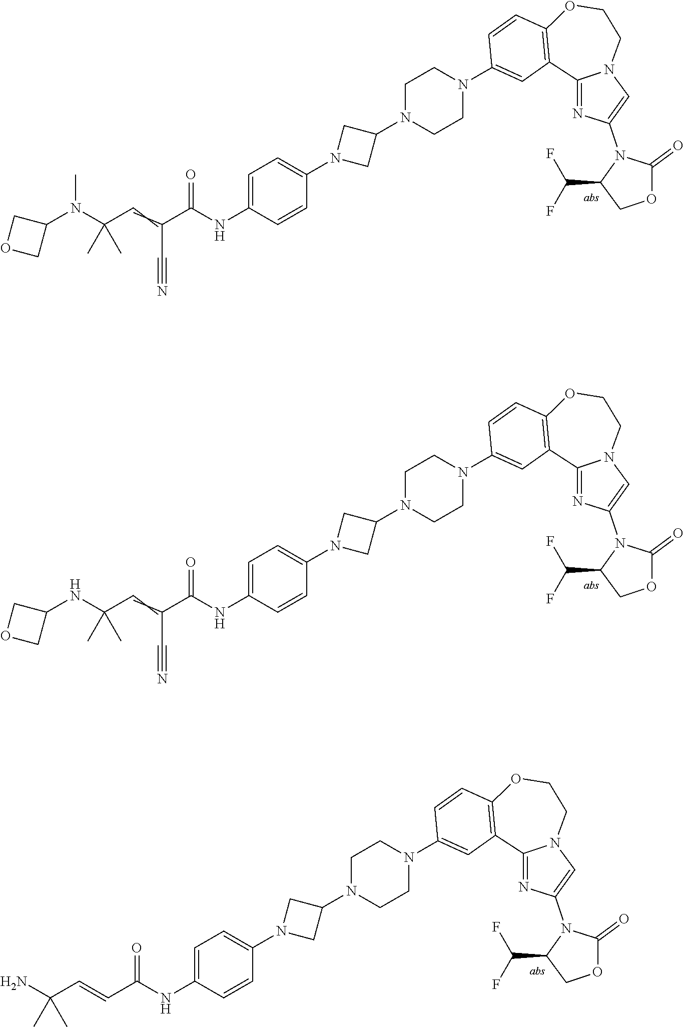 [SMILES: CN(C1COC1)C(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)NC1COC1 ; CC(C)(N)/C=C/C(=O)Nc1ccc(N2CC(N3CCN(c4ccc5c(c4)-c4nc(N6C(=O)OC[C@H]6C(F)F)cn4CCO5)CC3)C2)cc1]

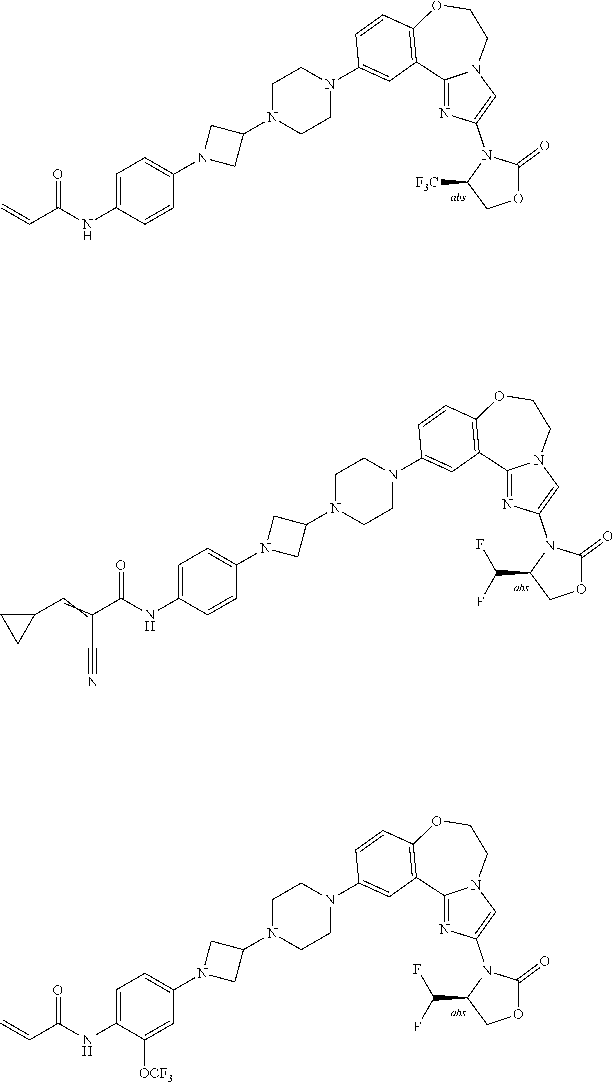 [SMILES: C=CC(=O)Nc1ccc(N2CC(N3CCN(c4ccc5c(c4)-c4nc(N6C(=O)OC[C@H]6C(F)(F)F)cn4CCO5)CC3)C2)cc1 ; N#CC(=CC1CC1)C(=O)Nc1ccc(N2CC(N3CCN(c4ccc5c(c4)-c4nc(N6C(=O)OC[C@H]6C(F)F)cn4CCO5)CC3)C2)cc1 ; C=CC(=O)Nc1ccc(N2CC(N3CCN(c4ccc5c(c4)-c4nc(N6C(=O)OC[C@H]6C(F)F)cn4CCO5)CC3)C2)cc1OC(F)(F)F]

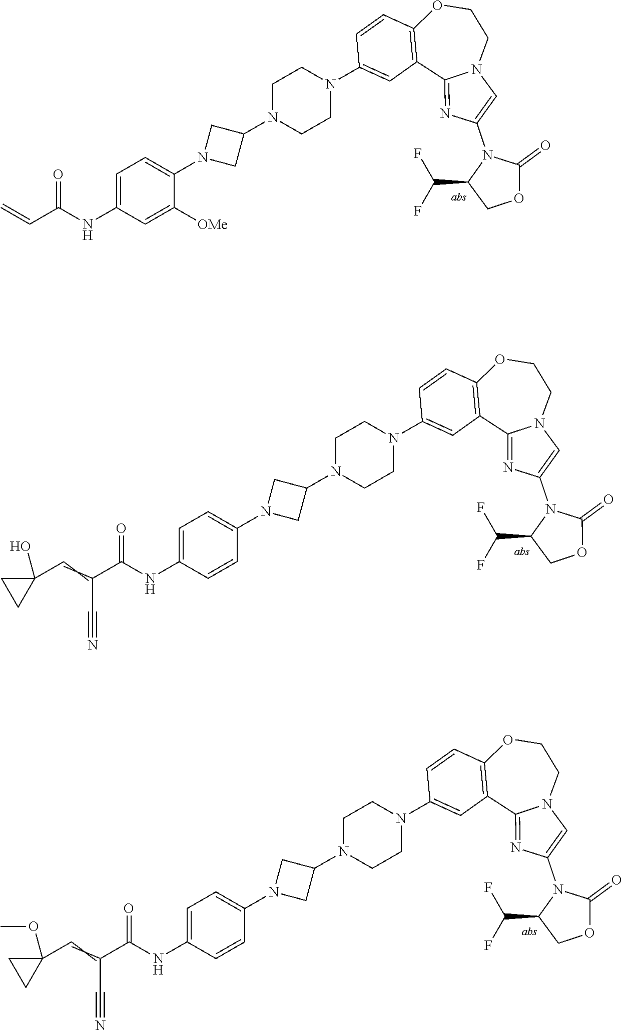 [SMILES: C=CC(=O)Nc1ccc(N2CC(N3CCN(c4ccc5c(c4)-c4nc(N6C(=O)OC[C@H]6C(F)F)cn4CCO5)CC3)C2)c(OC)c1 ; N#CC(=CC1(O)CC1)C(=O)Nc1ccc(N2CC(N3CCN(c4ccc5c(c4)-c4nc(N6C(=O)OC[C@H]6C(F)F)cn4CCO5)CC3)C2)cc1 ; COC1(C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CC1]

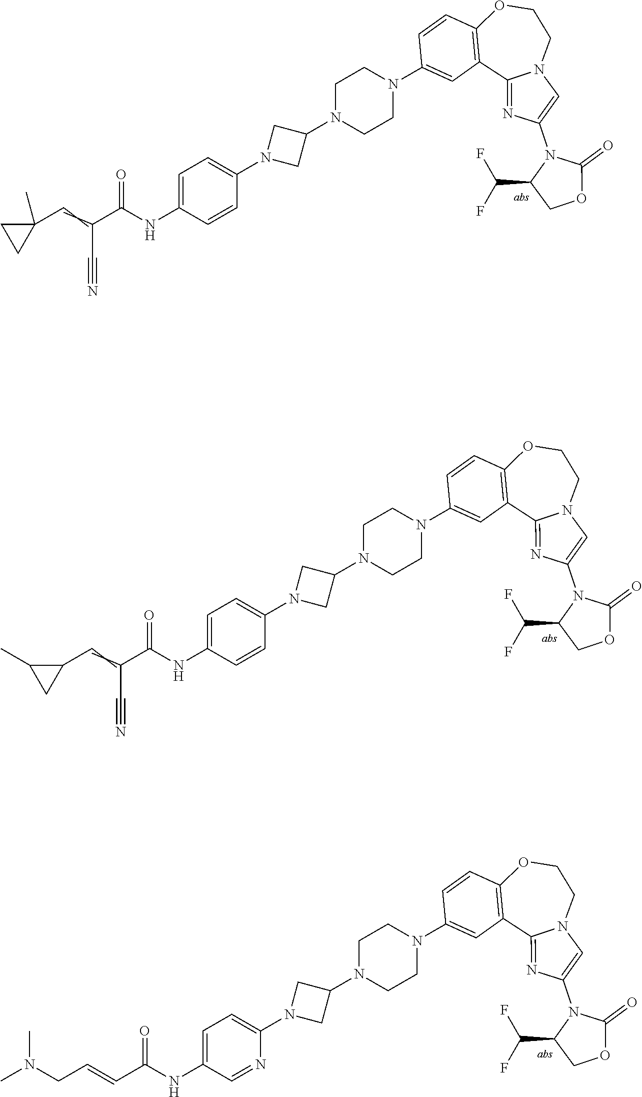 [SMILES: CC1(C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CC1 ; CC1CC1C=C(C#N)C(=O)Nc1ccc(N2CC(N3CCN(c4ccc5c(c4)-c4nc(N6C(=O)OC[C@H]6C(F)F)cn4CCO5)CC3)C2)cc1 ; CN(C)C/C=C/C(=O)Nc1ccc(N2CC(N3CCN(c4ccc5c(c4)-c4nc(N6C(=O)OC[C@H]6C(F)F)cn4CCO5)CC3)C2)nc1]

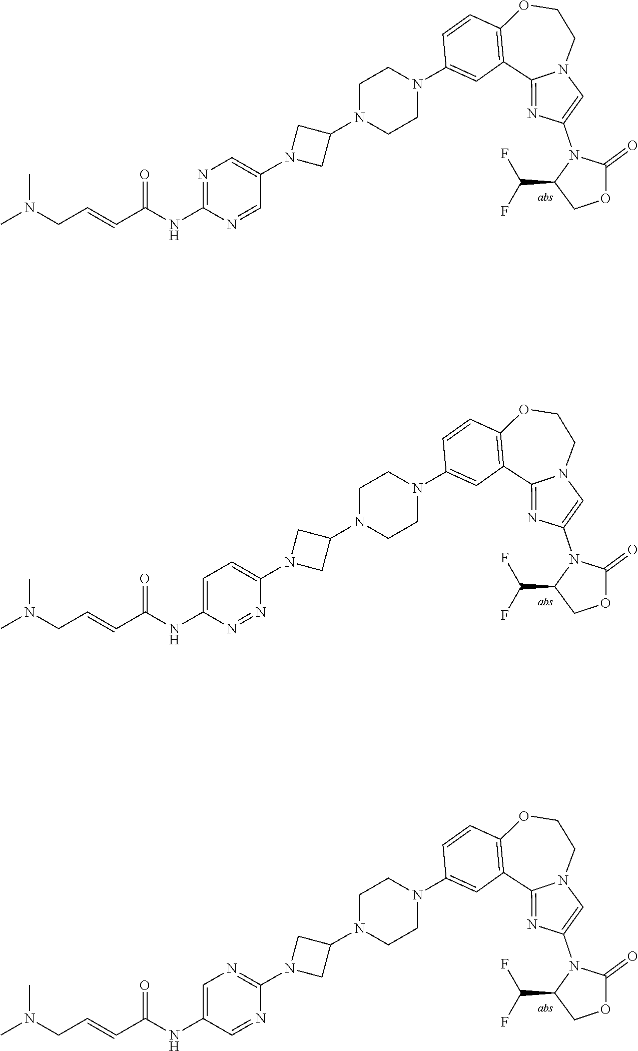 [SMILES: CN(C)C/C=C/C(=O)Nc1ncc(N2CC(N3CCN(c4ccc5c(c4)-c4nc(N6C(=O)OC[C@H]6C(F)F)cn4CCO5)CC3)C2)cn1 ; CN(C)C/C=C/C(=O)Nc1ccc(N2CC(N3CCN(c4ccc5c(c4)-c4nc(N6C(=O)OC[C@H]6C(F)F)cn4CCO5)CC3)C2)nn1 ; CN(C)C/C=C/C(=O)Nc1cnc(N2CC(N3CCN(c4ccc5c(c4)-c4nc(N6C(=O)OC[C@H]6C(F)F)cn4CCO5)CC3)C2)nc1]

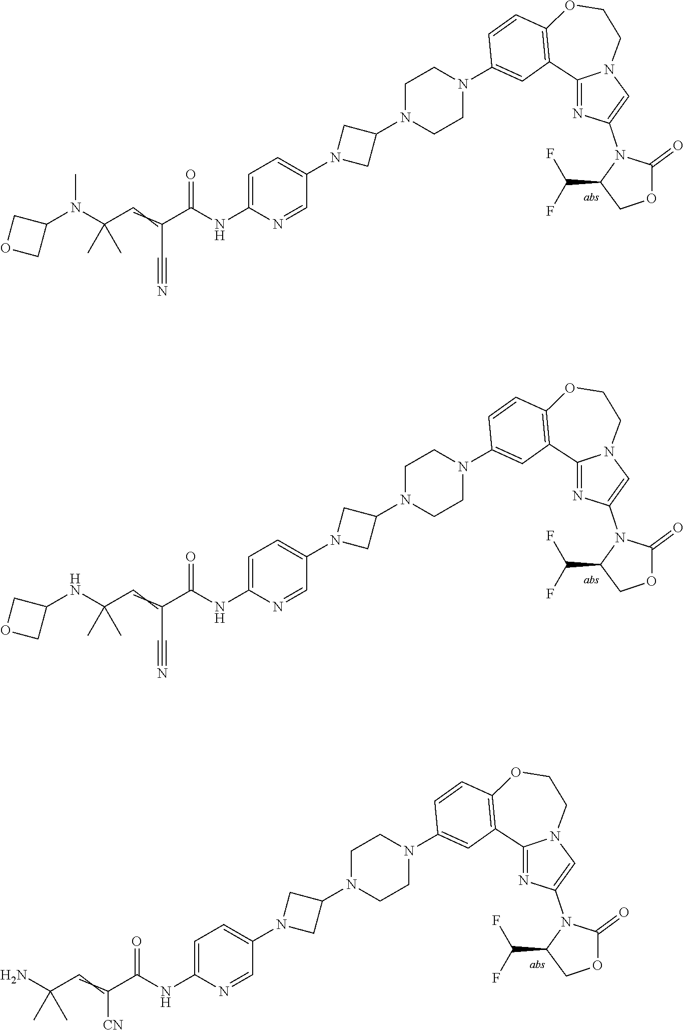 [SMILES: CN(C1COC1)C(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cn1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cn1)NC1COC1 ; CC(C)(N)C=C(C#N)C(=O)Nc1ccc(N2CC(N3CCN(c4ccc5c(c4)-c4nc(N6C(=O)OC[C@H]6C(F)F)cn4CCO5)CC3)C2)cn1]

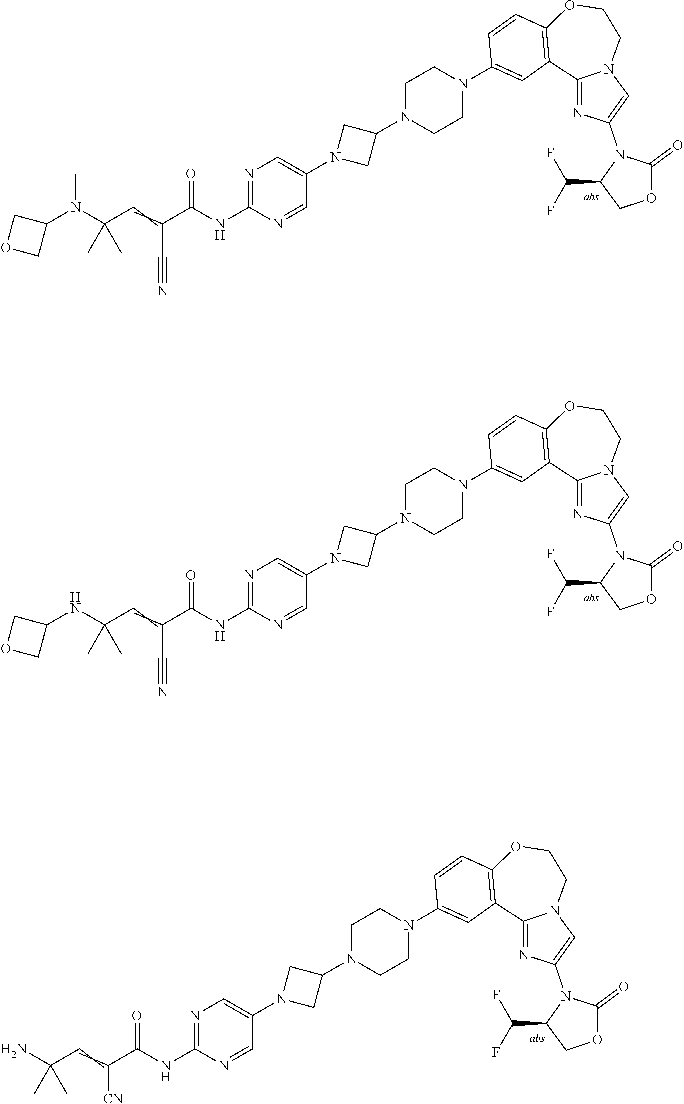 [SMILES: CN(C1COC1)C(C)(C)C=C(C#N)C(=O)Nc1ncc(N2CC(N3CCN(c4ccc5c(c4)-c4nc(N6C(=O)OC[C@H]6C(F)F)cn4CCO5)CC3)C2)cn1 ; CC(C)(C=C(C#N)C(=O)Nc1ncc(N2CC(N3CCN(c4ccc5c(c4)-c4nc(N6C(=O)OC[C@H]6C(F)F)cn4CCO5)CC3)C2)cn1)NC1COC1 ; CC(C)(N)C=C(C#N)C(=O)Nc1ncc(N2CC(N3CCN(c4ccc5c(c4)-c4nc(N6C(=O)OC[C@H]6C(F)F)cn4CCO5)CC3)C2)cn1]

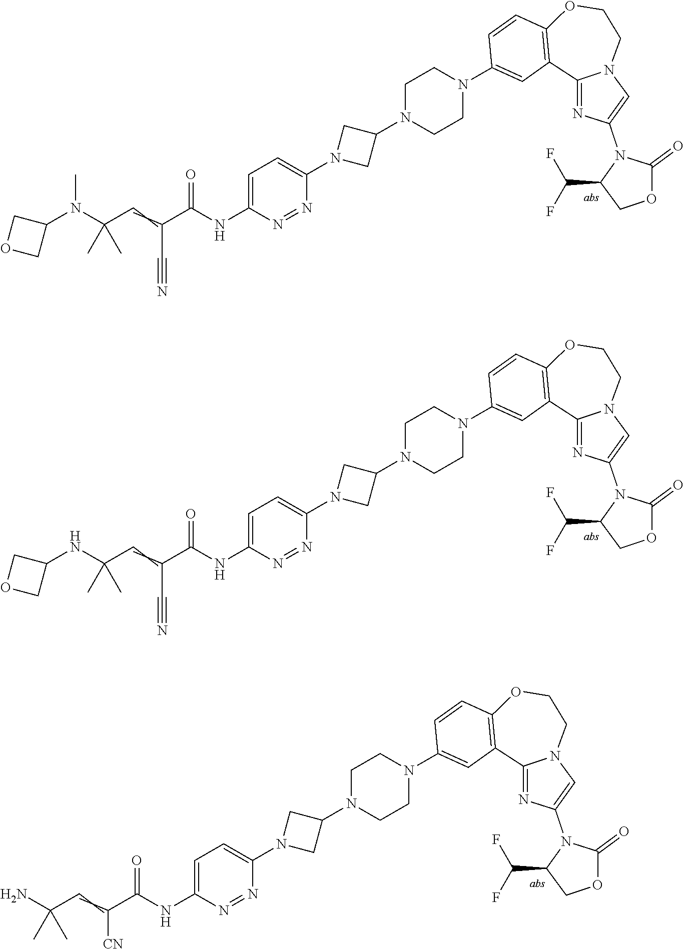 [SMILES: CN(C1COC1)C(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)nn1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)nn1)NC1COC1 ; CC(C)(N)C=C(C#N)C(=O)Nc1ccc(N2CC(N3CCN(c4ccc5c(c4)-c4nc(N6C(=O)OC[C@H]6C(F)F)cn4CCO5)CC3)C2)nn1]

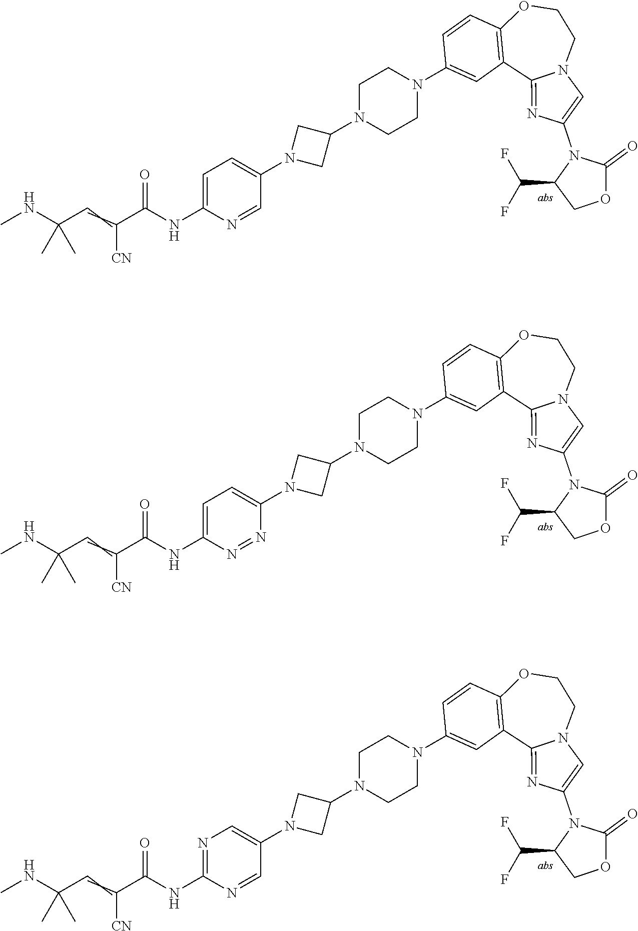 [SMILES: CNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cn1 ; CNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)nn1 ; CNC(C)(C)C=C(C#N)C(=O)Nc1ncc(N2CC(N3CCN(c4ccc5c(c4)-c4nc(N6C(=O)OC[C@H]6C(F)F)cn4CCO5)CC3)C2)cn1]

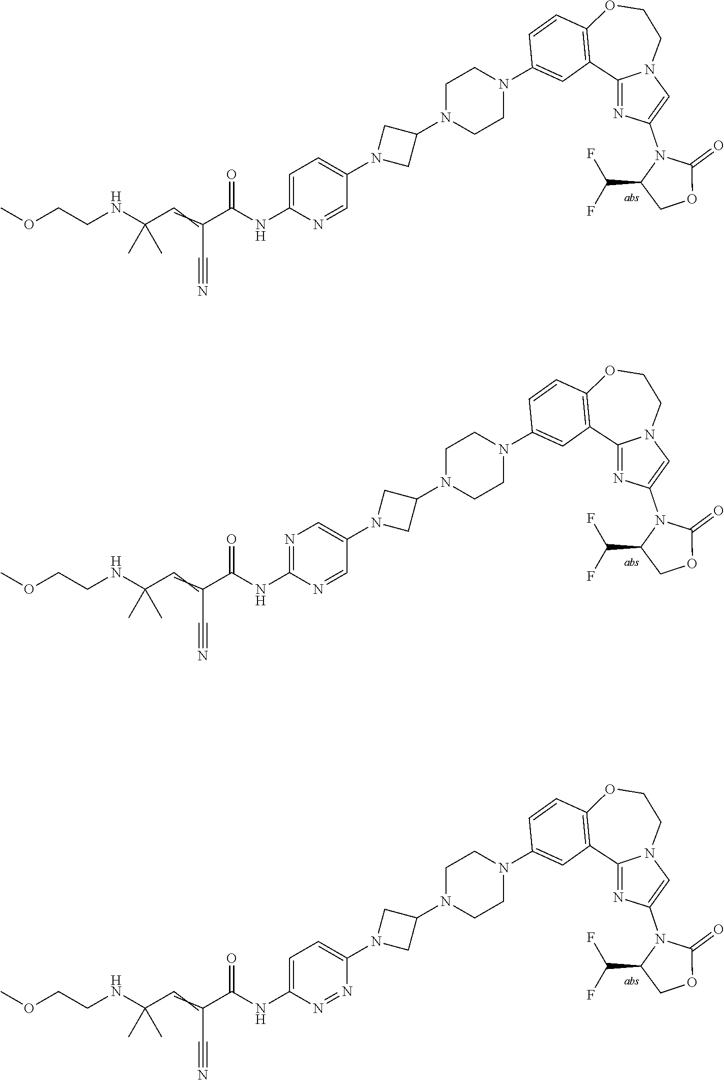 [SMILES: COCCNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cn1 ; COCCNC(C)(C)C=C(C#N)C(=O)Nc1ncc(N2CC(N3CCN(c4ccc5c(c4)-c4nc(N6C(=O)OC[C@H]6C(F)F)cn4CCO5)CC3)C2)cn1 ; COCCNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)nn1]

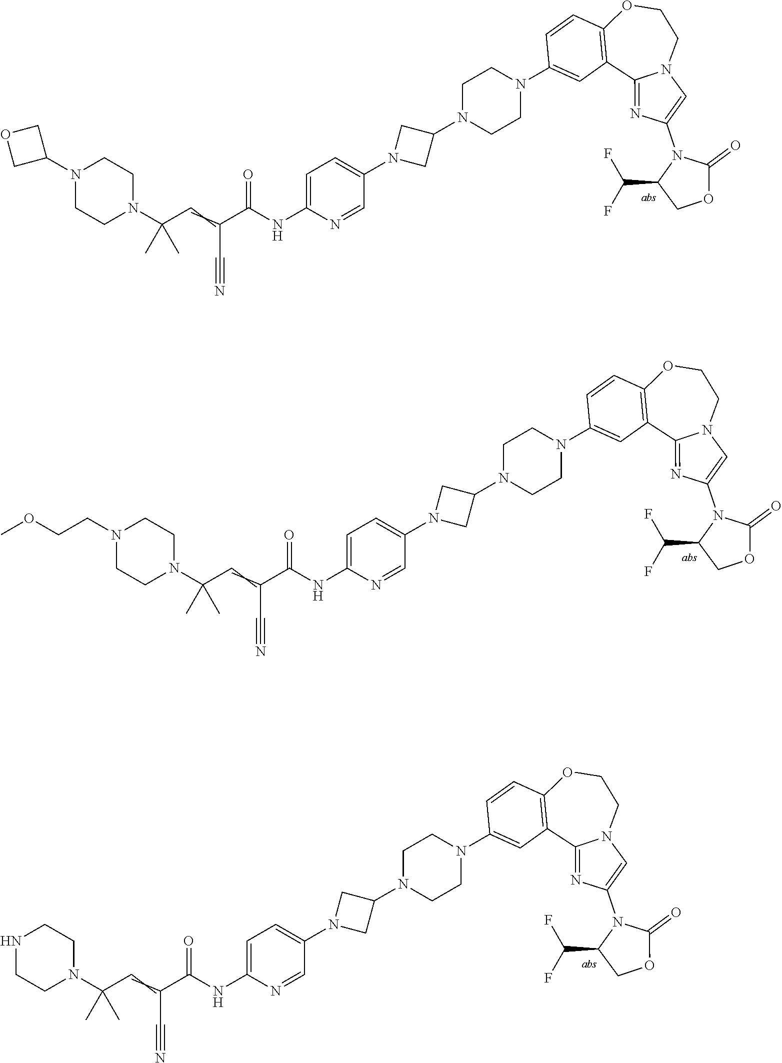 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cn1)N1CCN(C2COC2)CC1 ; COCCN1CCN(C(C)(C)C=C(C#N)C(=O)Nc2ccc(N3CC(N4CCN(c5ccc6c(c5)-c5nc(N7C(=O)OC[C@H]7C(F)F)cn5CCO6)CC4)C3)cn2)CC1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cn1)N1CCNCC1]

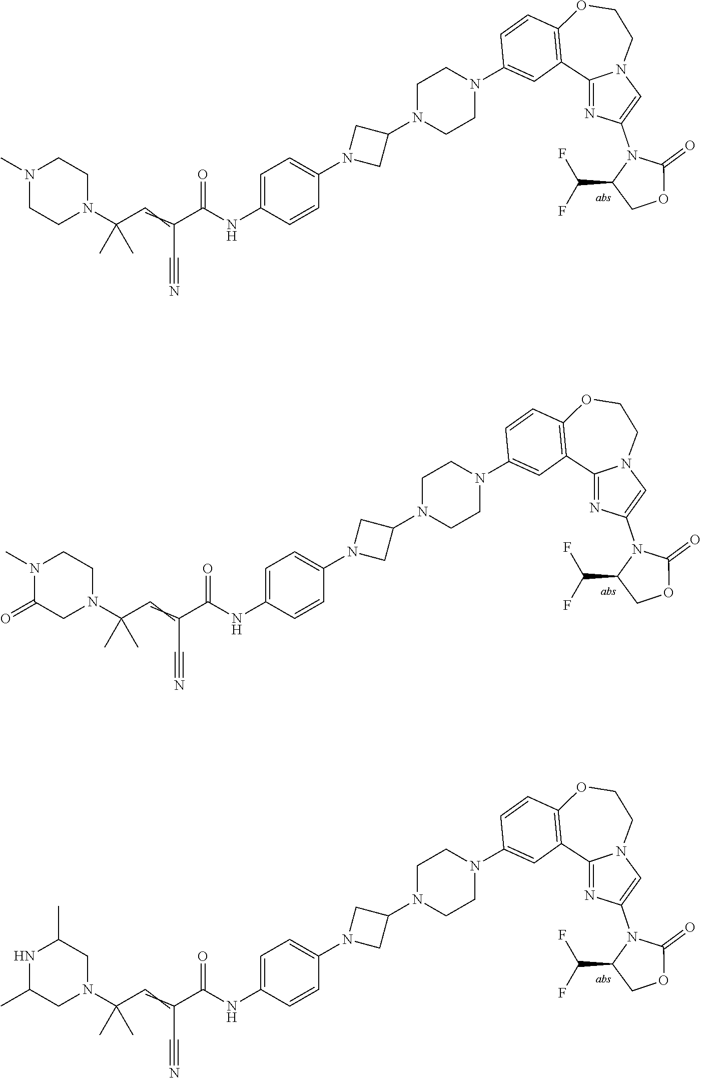 [SMILES: CN1CCN(C(C)(C)C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CC1 ; CN1CCN(C(C)(C)C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CC1=O ; CC1CN(C(C)(C)C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CC(C)N1]

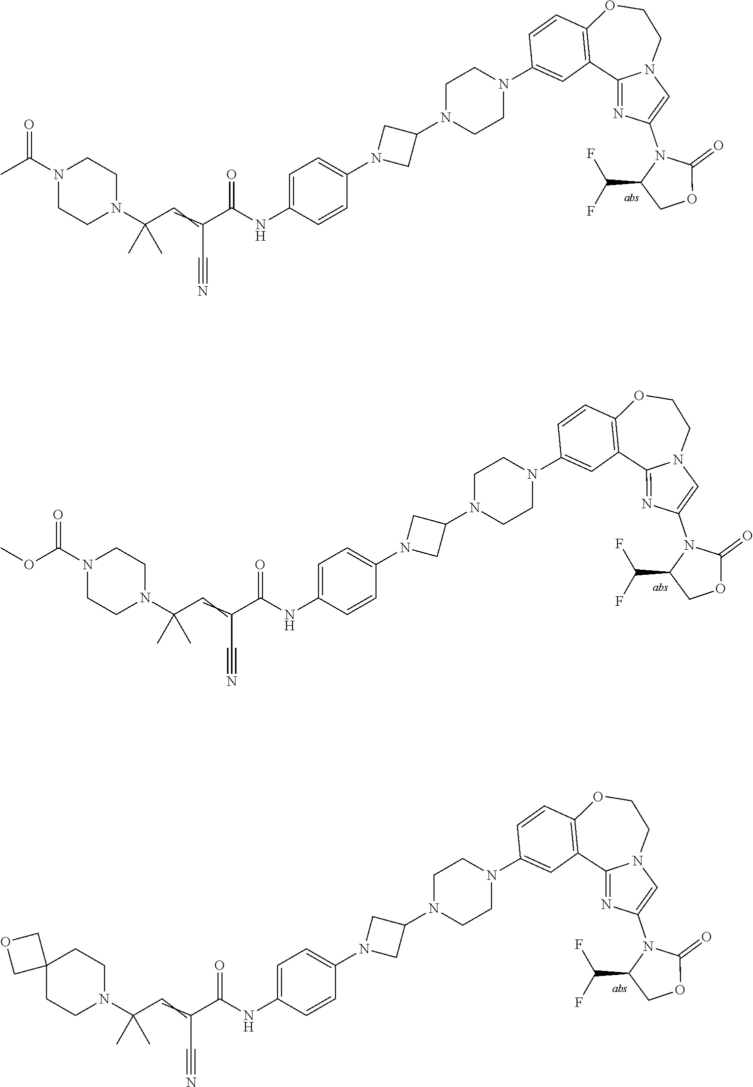 [SMILES: CC(=O)N1CCN(C(C)(C)C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CC1 ; COC(=O)N1CCN(C(C)(C)C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CC1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)N1CCC2(CC1)COC2]

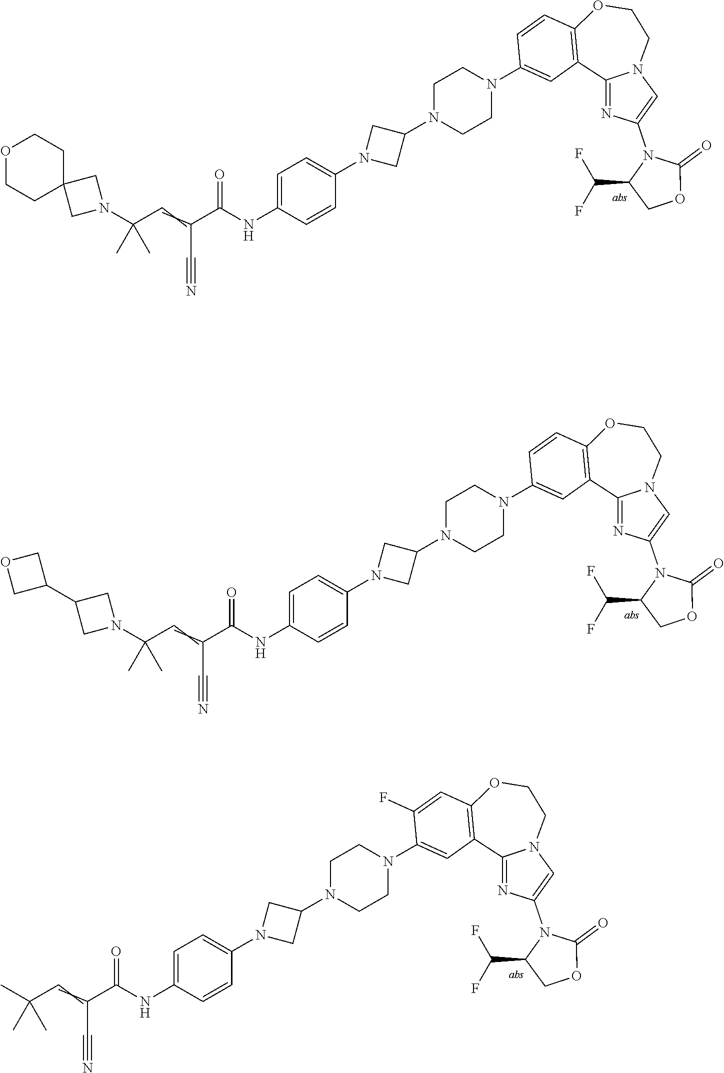 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)N1CC2(CCOCC2)C1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)N1CC(C2COC2)C1 ; CC(C)(C)C=C(C#N)C(=O)Nc1ccc(N2CC(N3CCN(c4cc5c(cc4F)OCCn4cc(N6C(=O)OC[C@H]6C(F)F)nc4-5)CC3)C2)cc1]

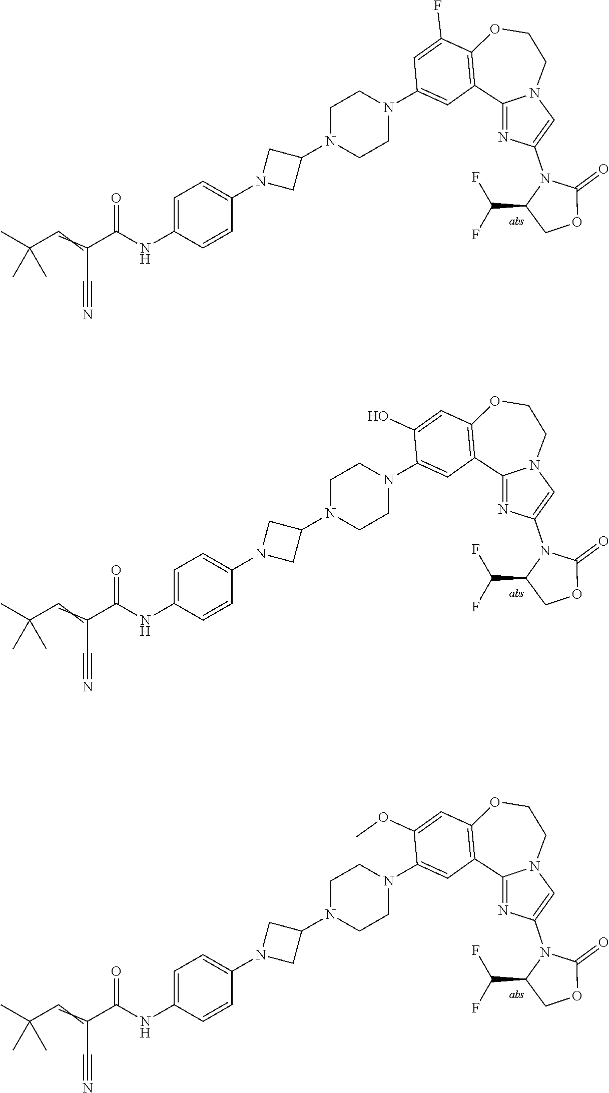 [SMILES: CC(C)(C)C=C(C#N)C(=O)Nc1ccc(N2CC(N3CCN(c4cc(F)c5c(c4)-c4nc(N6C(=O)OC[C@H]6C(F)F)cn4CCO5)CC3)C2)cc1 ; CC(C)(C)C=C(C#N)C(=O)Nc1ccc(N2CC(N3CCN(c4cc5c(cc4O)OCCn4cc(N6C(=O)OC[C@H]6C(F)F)nc4-5)CC3)C2)cc1 ; COc1cc2c(cc1N1CCN(C3CN(c4ccc(NC(=O)C(C#N)=CC(C)(C)C)cc4)C3)CC1)-c1nc(N3C(=O)OC[C@H]3C(F)F)cn1CCO2]

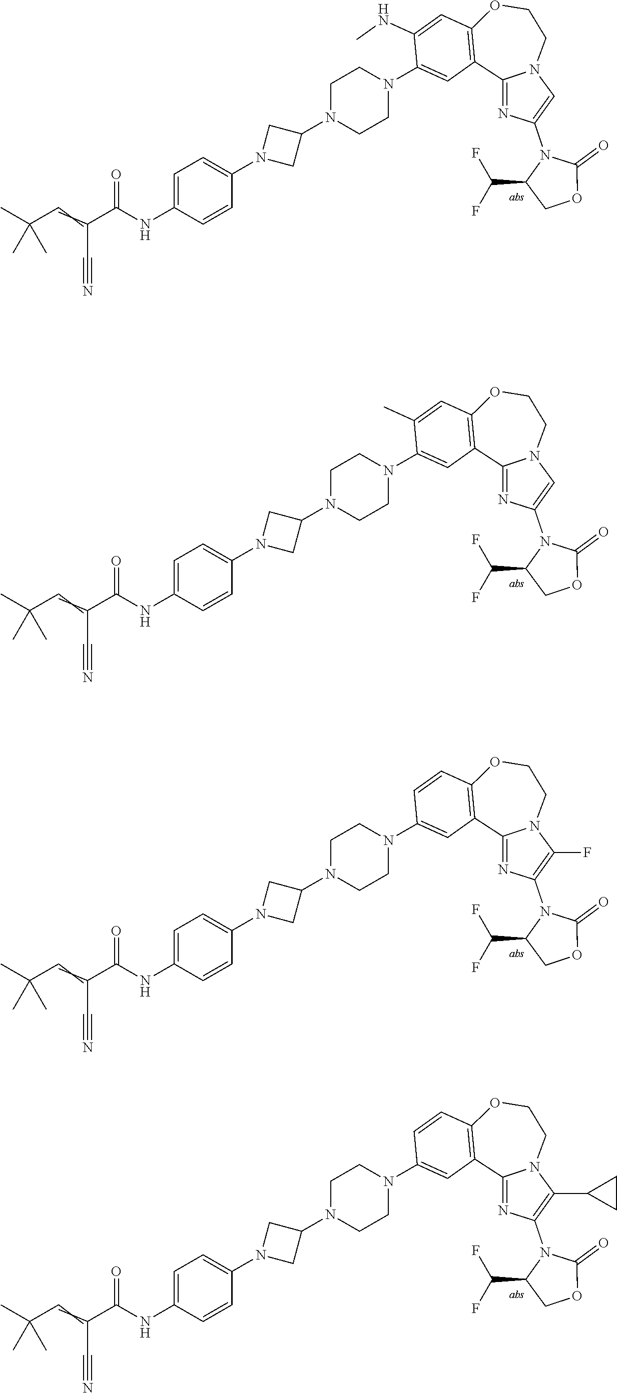 [SMILES: CNc1cc2c(cc1N1CCN(C3CN(c4ccc(NC(=O)C(C#N)=CC(C)(C)C)cc4)C3)CC1)-c1nc(N3C(=O)OC[C@H]3C(F)F)cn1CCO2 ; Cc1cc2c(cc1N1CCN(C3CN(c4ccc(NC(=O)C(C#N)=CC(C)(C)C)cc4)C3)CC1)-c1nc(N3C(=O)OC[C@H]3C(F)F)cn1CCO2 ; CC(C)(C)C=C(C#N)C(=O)Nc1ccc(N2CC(N3CCN(c4ccc5c(c4)-c4nc(N6C(=O)OC[C@H]6C(F)F)c(F)n4CCO5)CC3)C2)cc1 ; CC(C)(C)C=C(C#N)C(=O)Nc1ccc(N2CC(N3CCN(c4ccc5c(c4)-c4nc(N6C(=O)OC[C@H]6C(F)F)c(C6CC6)n4CCO5)CC3)C2)cc1]

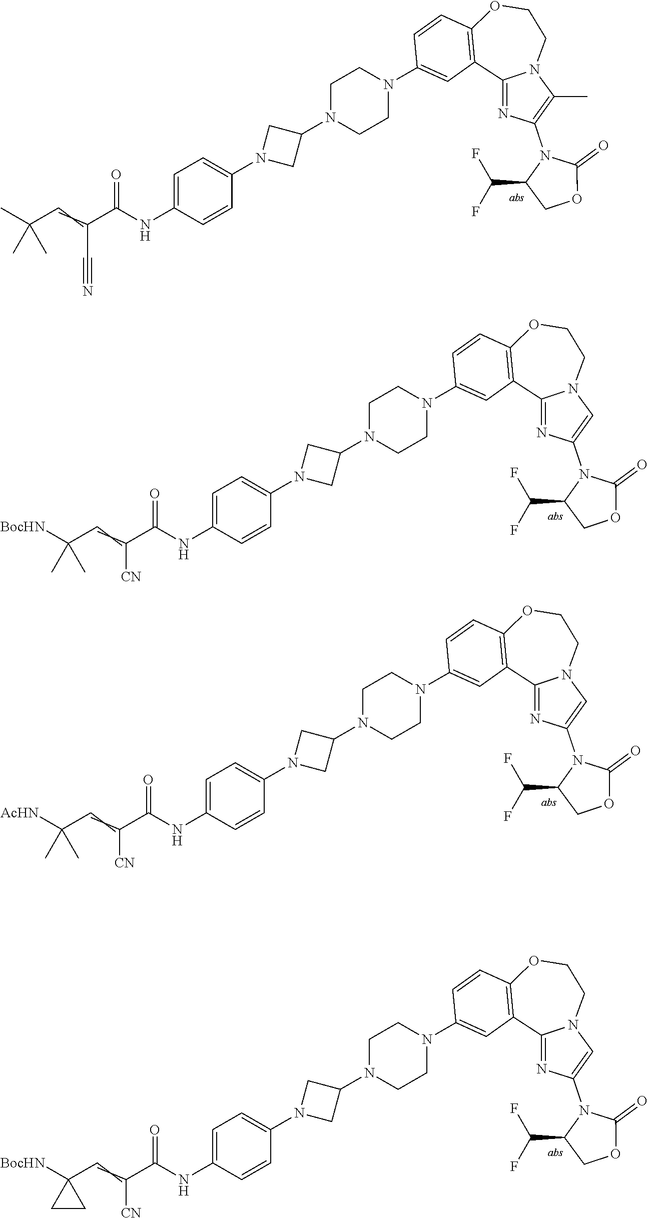 [SMILES: Cc1c(N2C(=O)OC[C@H]2C(F)F)nc2n1CCOc1ccc(N3CCN(C4CN(c5ccc(NC(=O)C(C#N)=CC(C)(C)C)cc5)C4)CC3)cc1-2 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)NC(=O)OC(C)(C)C ; CC(=O)NC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1 ; CC(C)(C)OC(=O)NC1(C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CC1]

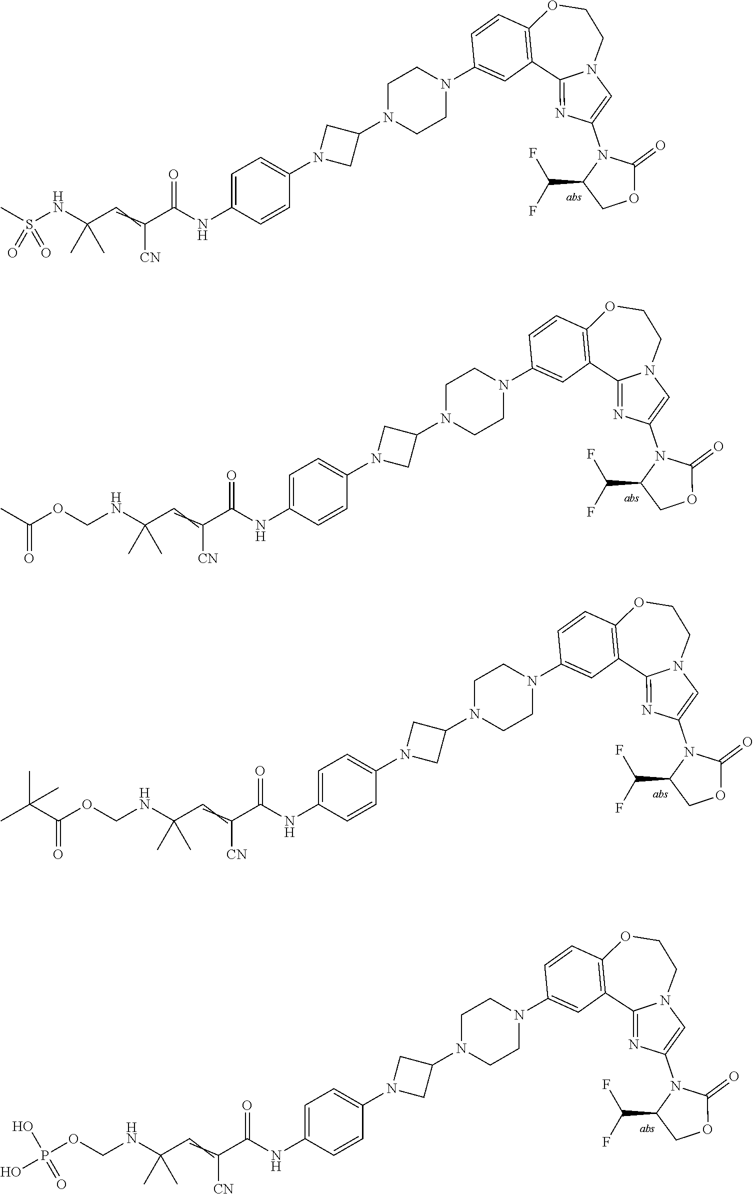 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)NS(C)(=O)=O ; CC(=O)OCNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)NCOC(=O)C(C)(C)C ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)NCOP(=O)(O)O]

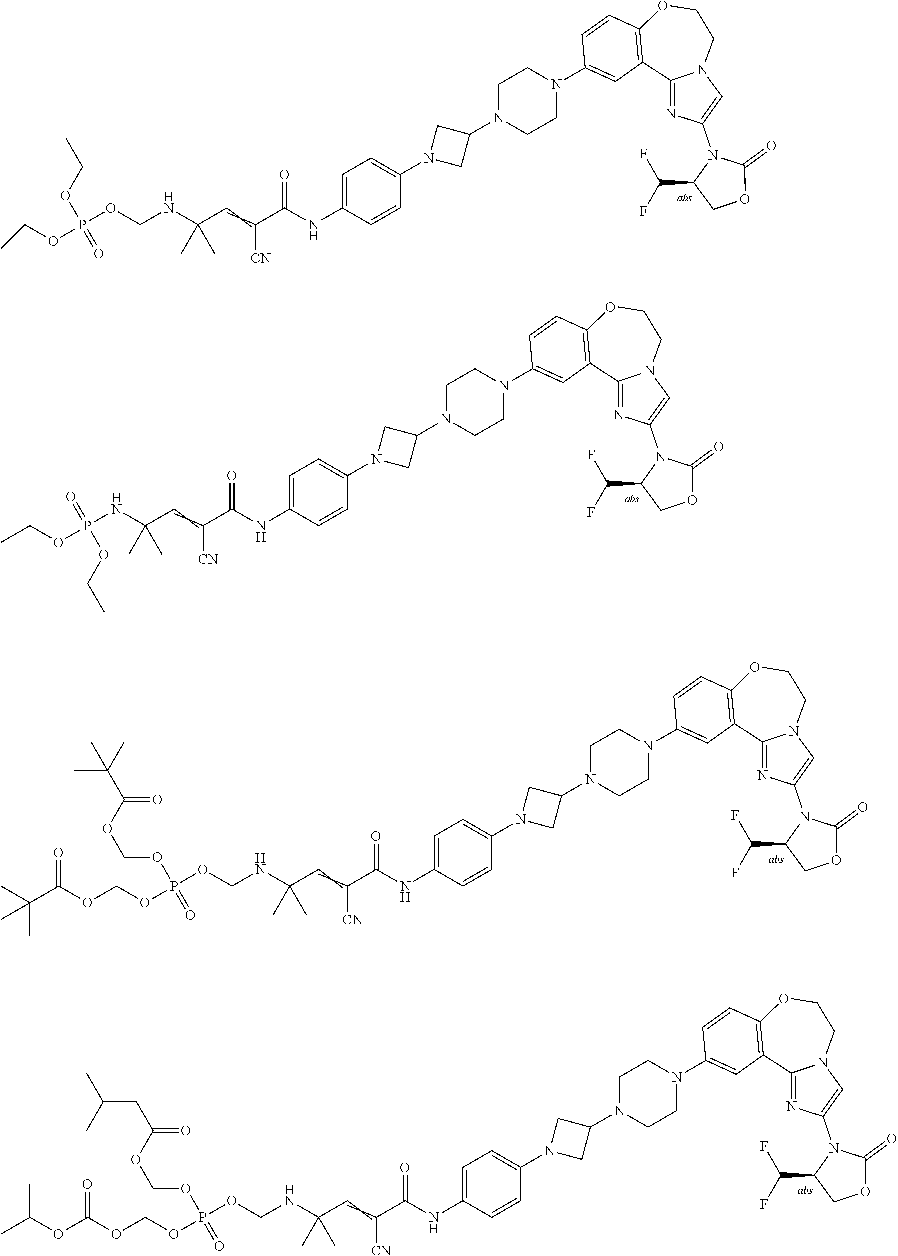 [SMILES: CCOP(=O)(OCC)OCNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1 ; CCOP(=O)(NC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1)OCC ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)NCOP(=O)(OCOC(=O)C(C)(C)C)OCOC(=O)C(C)(C)C ; CC(C)CC(=O)OCOP(=O)(OCNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1)OCOC(=O)OC(C)C]

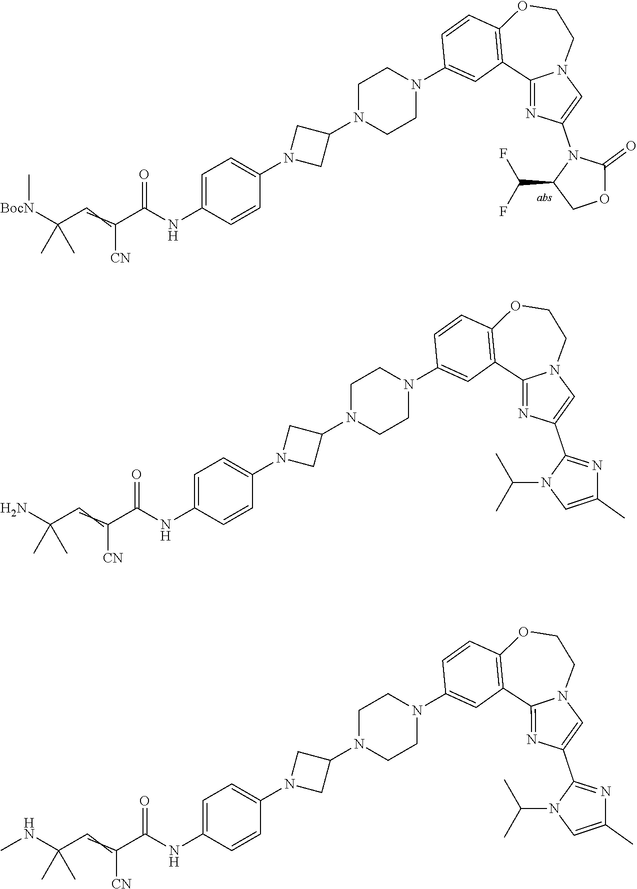

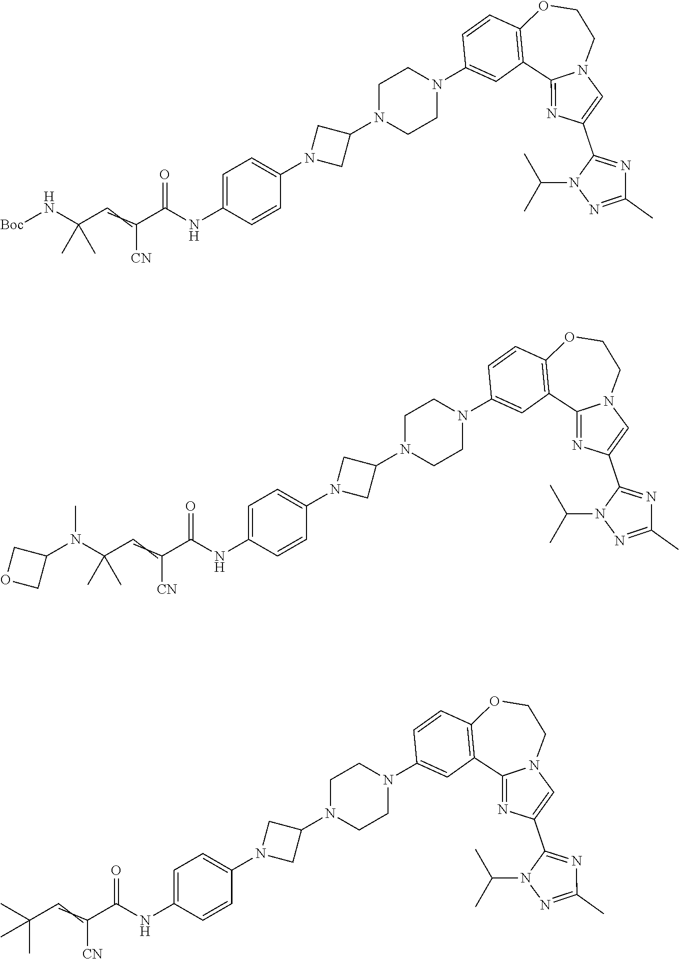

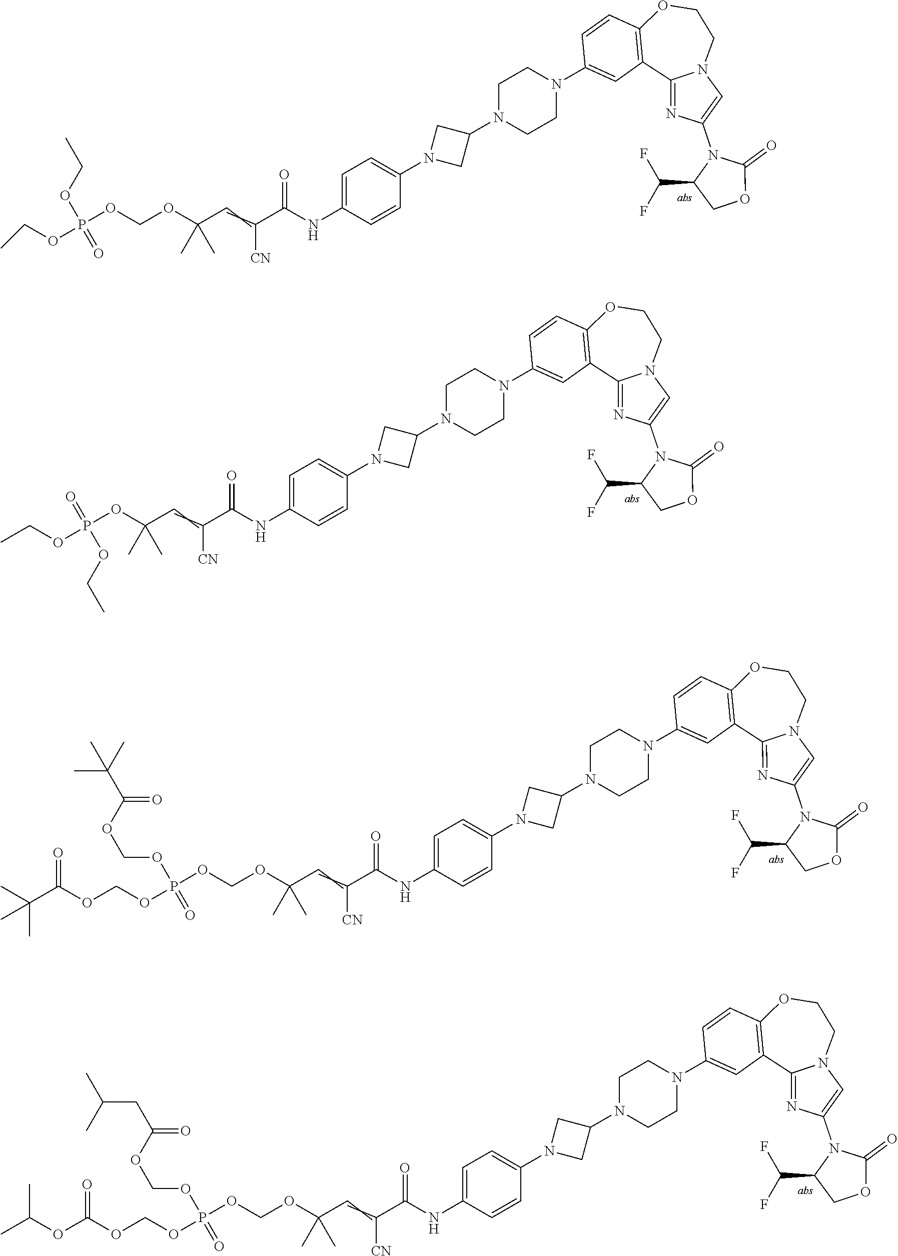 [SMILES: CCOP(=O)(OCC)OCOC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1 ; CCOP(=O)(OCC)OC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)OCOP(=O)(OCOC(=O)C(C)(C)C)OCOC(=O)C(C)(C)C ; CC(C)CC(=O)OCOP(=O)(OCOC(=O)OC(C)C)OCOC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1]

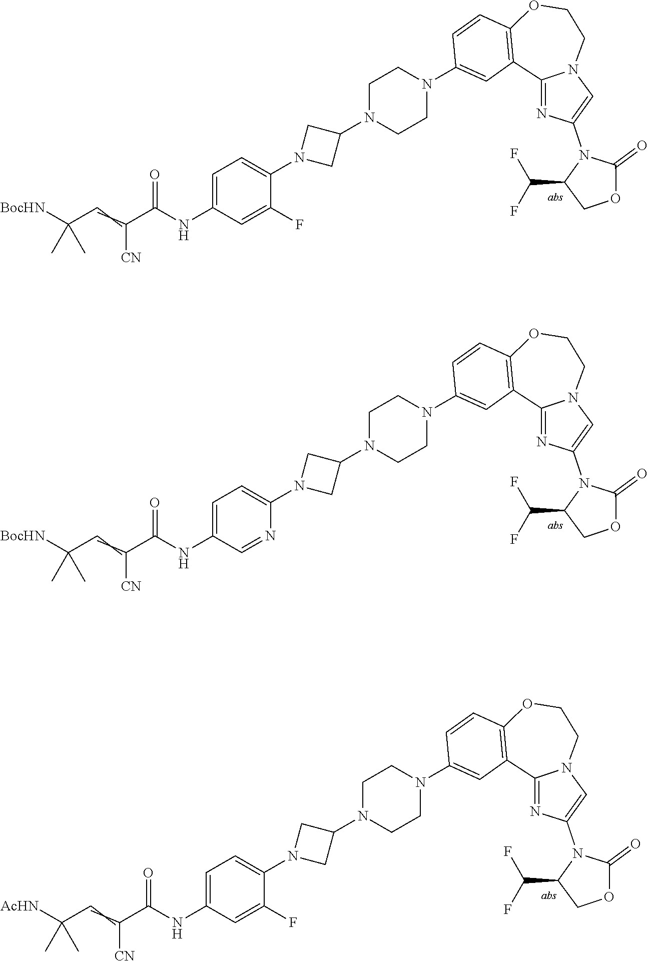 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1)NC(=O)OC(C)(C)C ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)nc1)NC(=O)OC(C)(C)C ; CC(=O)NC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1]

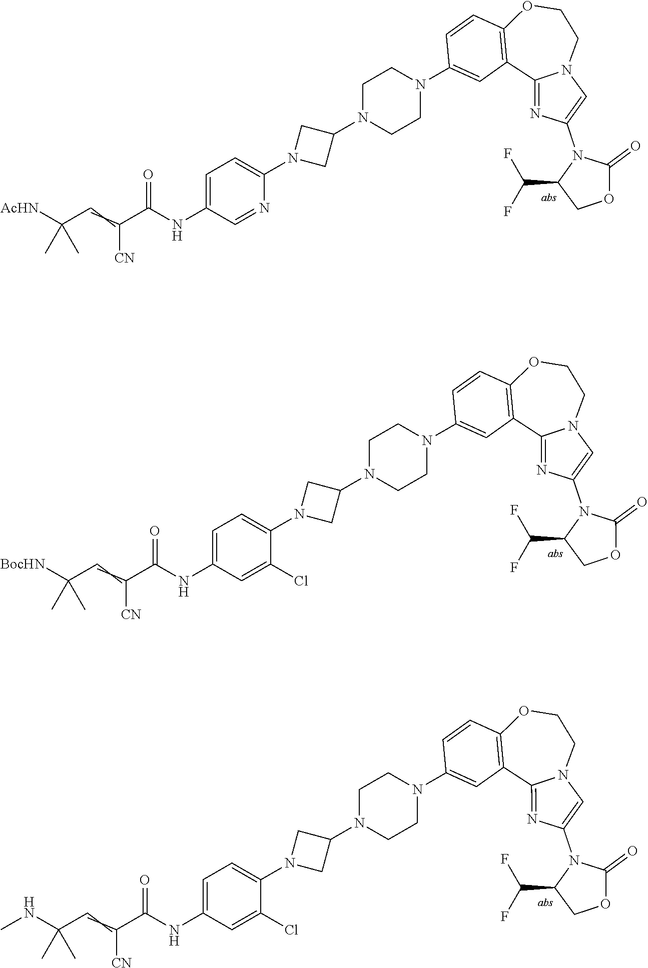 [SMILES: CC(=O)NC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)nc1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)c(Cl)c1)NC(=O)OC(C)(C)C ; CNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)c(Cl)c1]

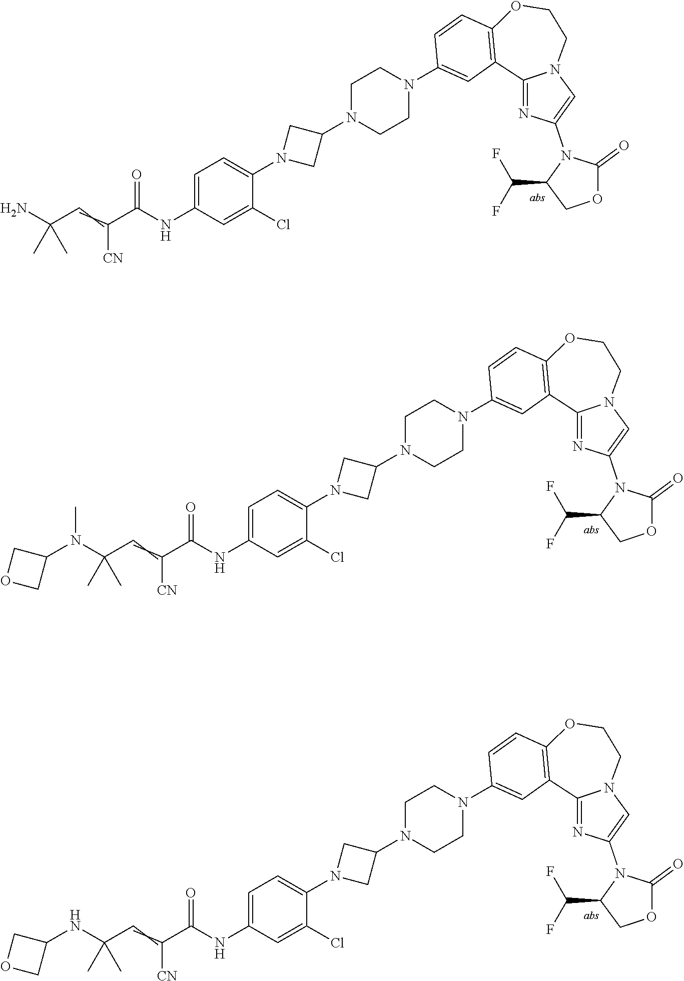 [SMILES: CC(C)(N)C=C(C#N)C(=O)Nc1ccc(N2CC(N3CCN(c4ccc5c(c4)-c4nc(N6C(=O)OC[C@H]6C(F)F)cn4CCO5)CC3)C2)c(Cl)c1 ; CN(C1COC1)C(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)c(Cl)c1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)c(Cl)c1)NC1COC1]

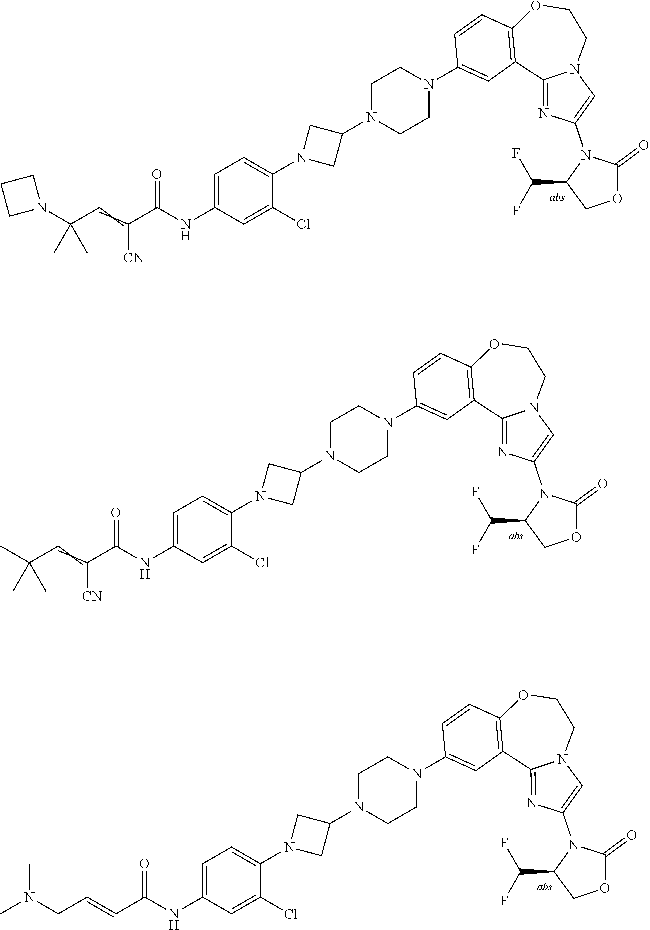 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)c(Cl)c1)N1CCC1 ; CC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)c(Cl)c1 ; CN(C)C/C=C/C(=O)Nc1ccc(N2CC(N3CCN(c4ccc5c(c4)-c4nc(N6C(=O)OC[C@H]6C(F)F)cn4CCO5)CC3)C2)c(Cl)c1]

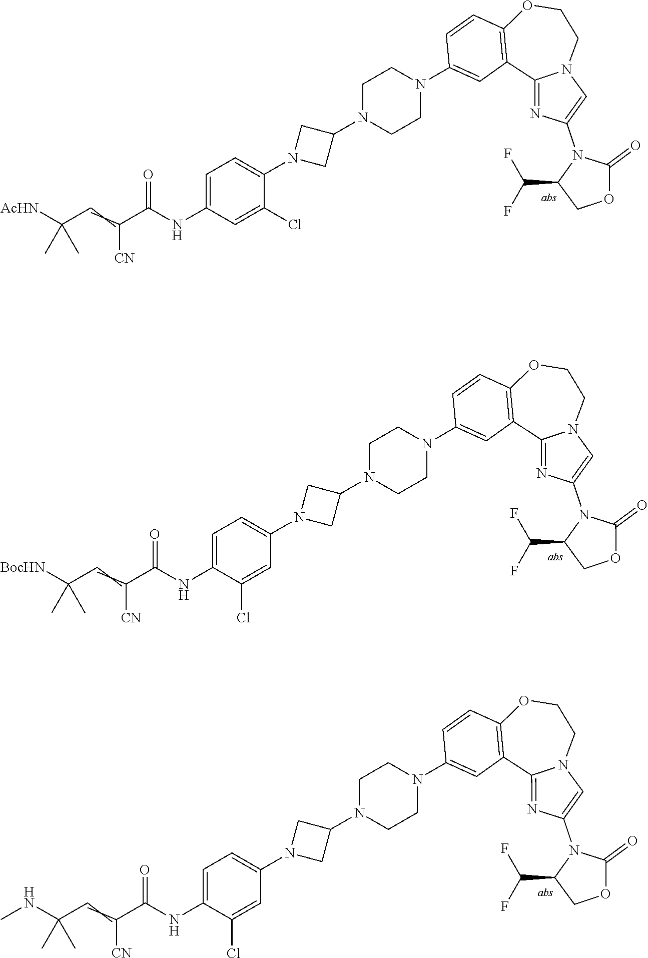 [SMILES: CC(=O)NC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)c(Cl)c1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1Cl)NC(=O)OC(C)(C)C ; CNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1Cl]

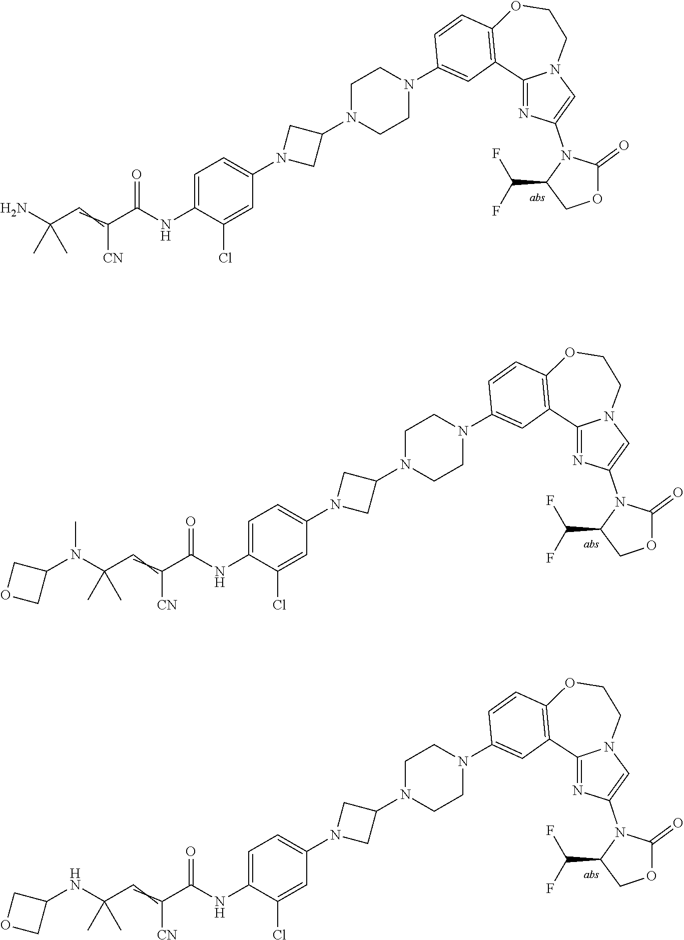 [SMILES: CC(C)(N)C=C(C#N)C(=O)Nc1ccc(N2CC(N3CCN(c4ccc5c(c4)-c4nc(N6C(=O)OC[C@H]6C(F)F)cn4CCO5)CC3)C2)cc1Cl ; CN(C1COC1)C(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1Cl ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1Cl)NC1COC1]

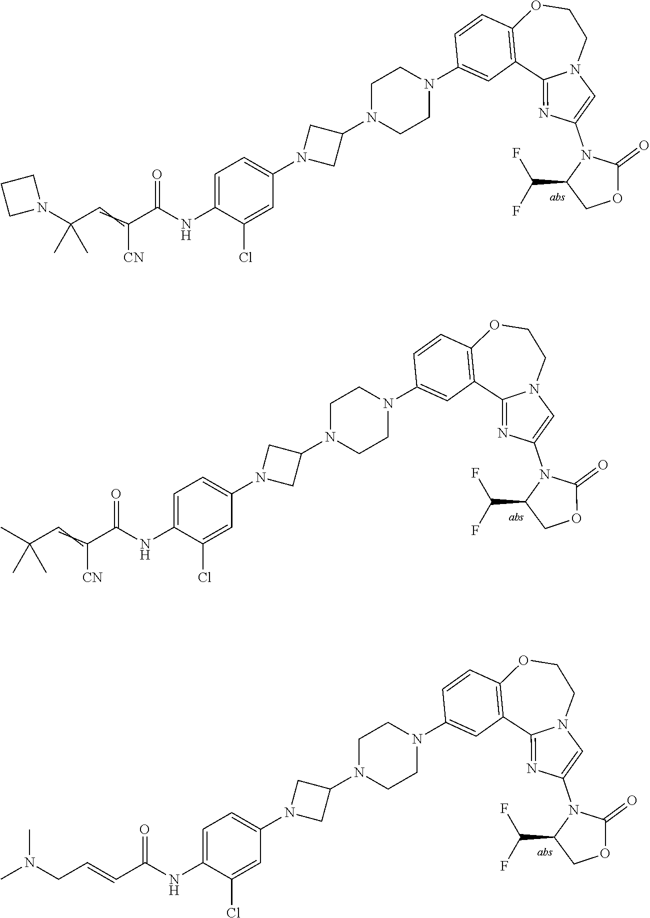 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1Cl)N1CCC1 ; CC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1Cl ; CN(C)C/C=C/C(=O)Nc1ccc(N2CC(N3CCN(c4ccc5c(c4)-c4nc(N6C(=O)OC[C@H]6C(F)F)cn4CCO5)CC3)C2)cc1Cl]

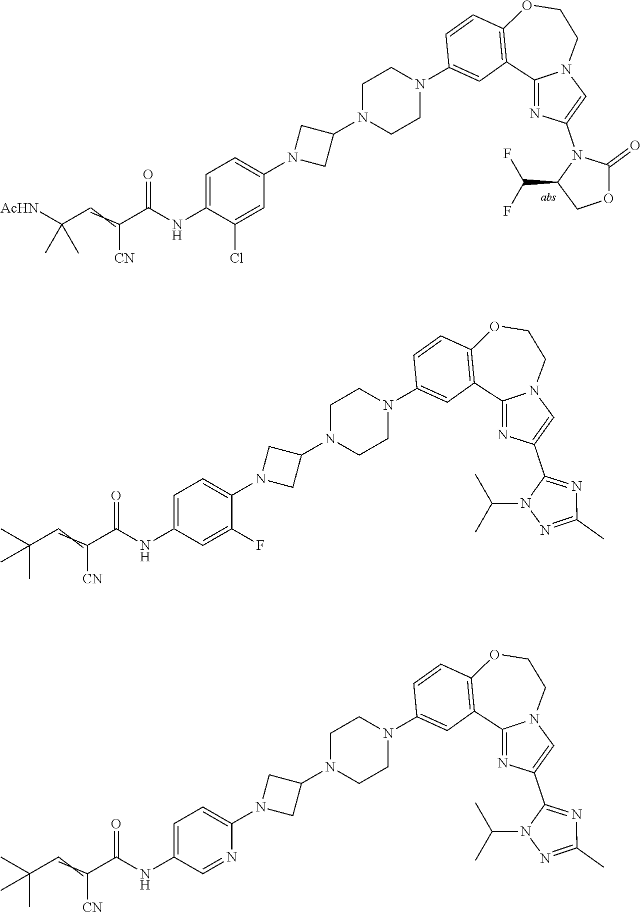

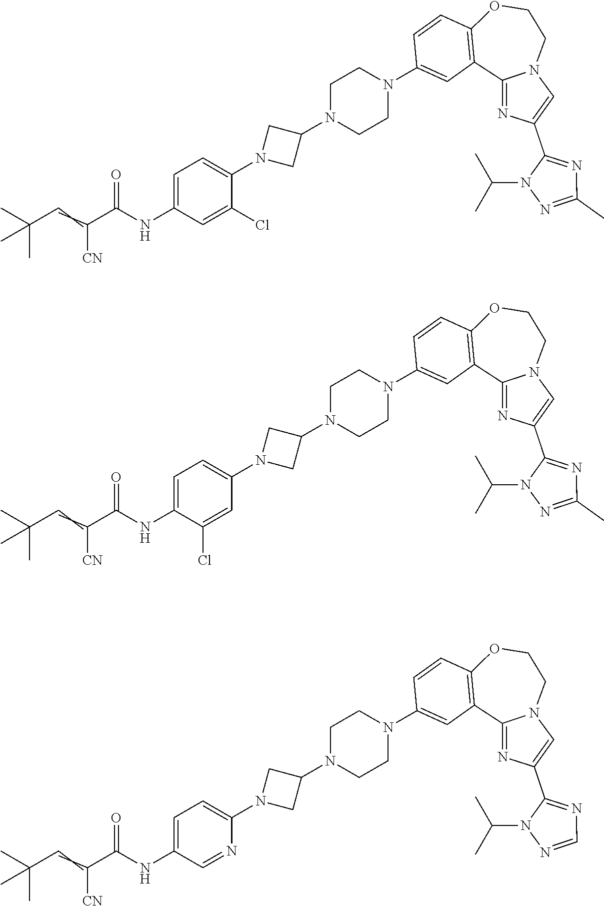

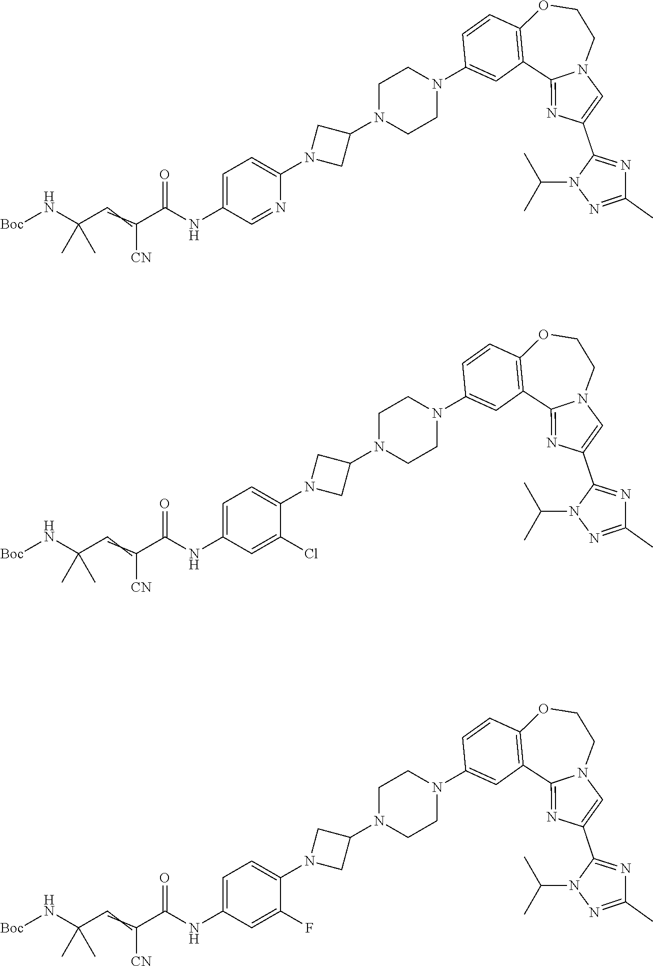 [SMILES: Cc1nc(-c2cn3c(n2)-c2cc(N4CCN(C5CN(c6ccc(NC(=O)C(C#N)=CC(C)(C)NC(=O)OC(C)(C)C)cn6)C5)CC4)ccc2OCC3)n(C(C)C)n1 ; Cc1nc(-c2cn3c(n2)-c2cc(N4CCN(C5CN(c6ccc(NC(=O)C(C#N)=CC(C)(C)NC(=O)OC(C)(C)C)cc6Cl)C5)CC4)ccc2OCC3)n(C(C)C)n1 ; Cc1nc(-c2cn3c(n2)-c2cc(N4CCN(C5CN(c6ccc(NC(=O)C(C#N)=CC(C)(C)NC(=O)OC(C)(C)C)cc6F)C5)CC4)ccc2OCC3)n(C(C)C)n1]

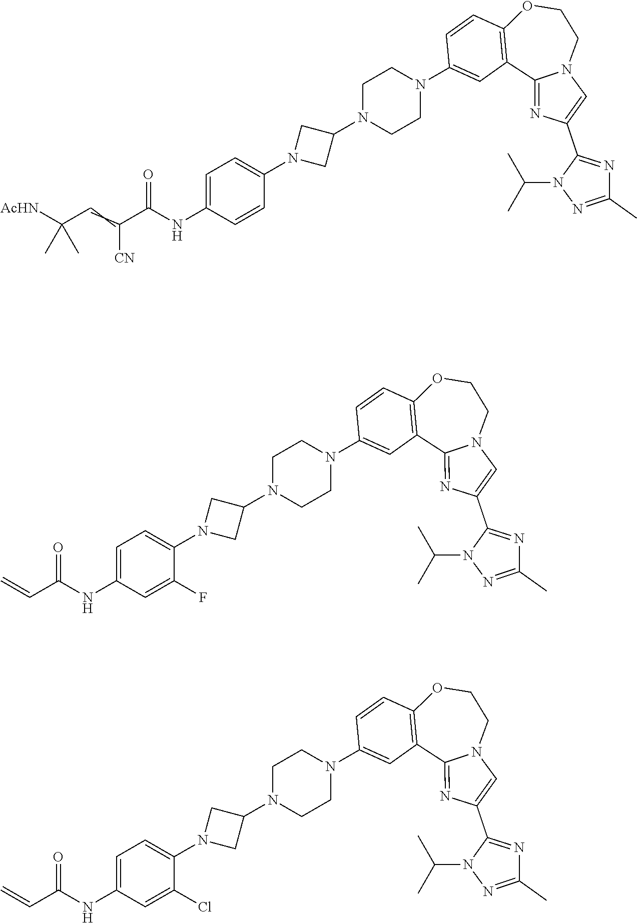

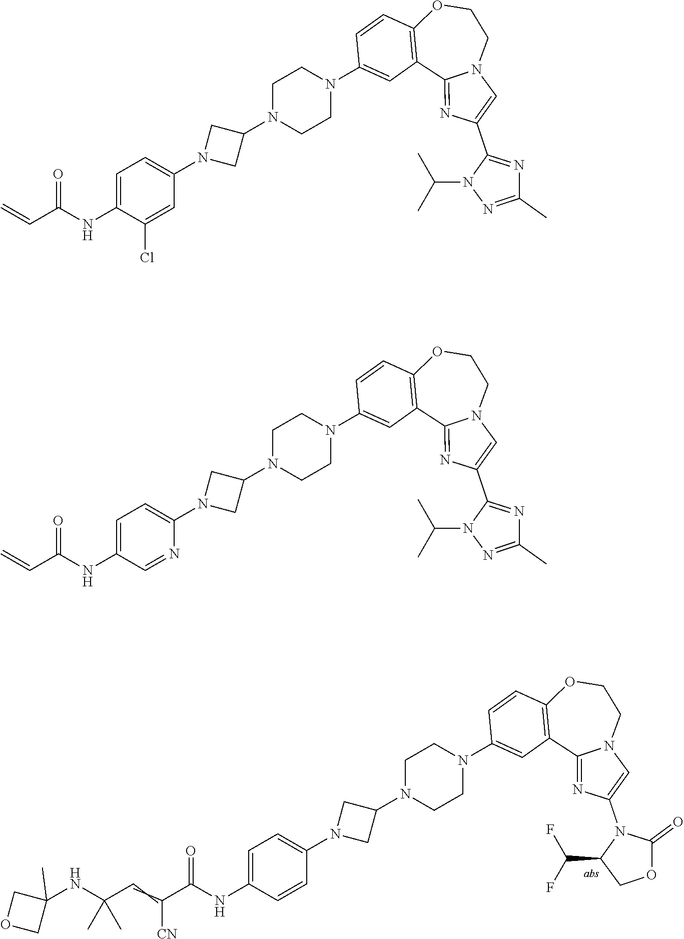

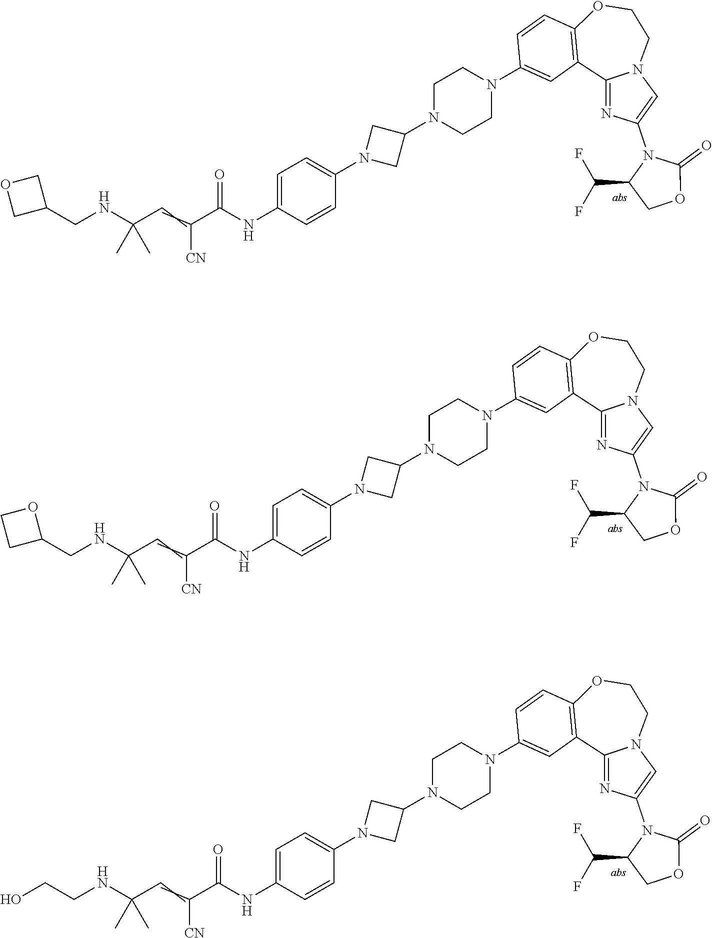 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)NCC1COC1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)NCC1CCO1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)NCCO]

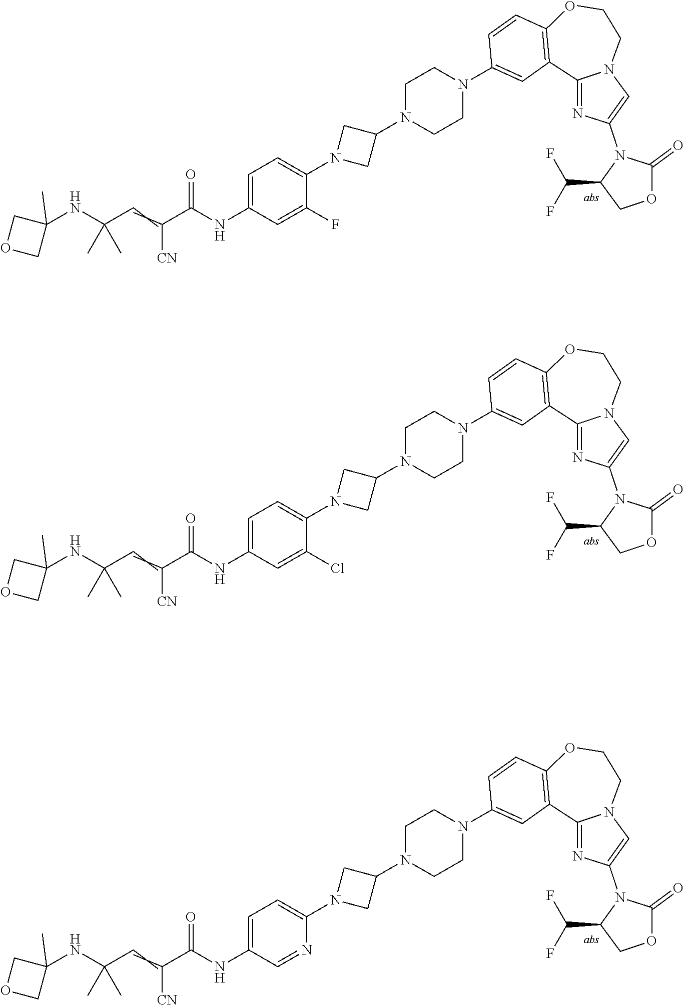 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1)NC1(C)COC1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)c(Cl)c1)NC1(C)COC1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)nc1)NC1(C)COC1]

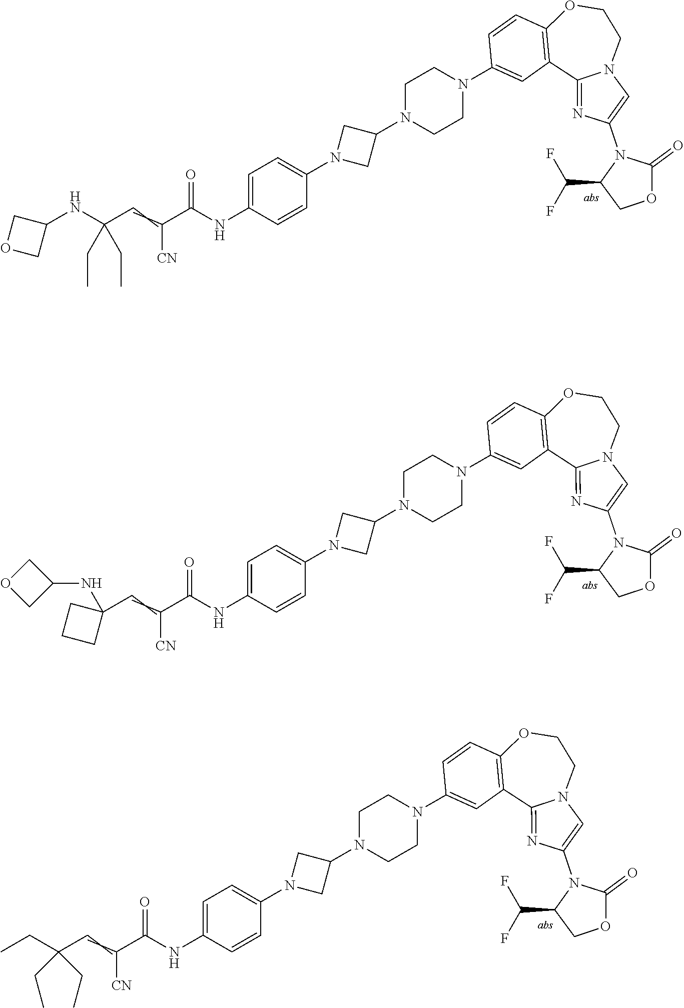 [SMILES: CCC(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)(CC)NC1COC1 ; N#CC(=CC1(NC2COC2)CCC1)C(=O)Nc1ccc(N2CC(N3CCN(c4ccc5c(c4)-c4nc(N6C(=O)OC[C@H]6C(F)F)cn4CCO5)CC3)C2)cc1 ; CCC(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)(CC)CC]

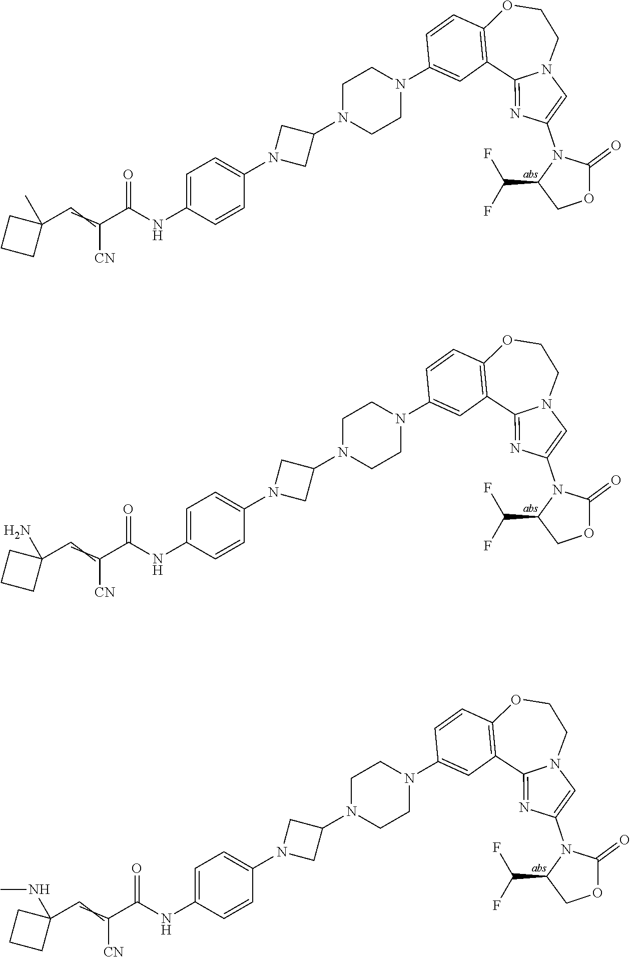 [SMILES: CC1(C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CCC1 ; N#CC(=CC1(N)CCC1)C(=O)Nc1ccc(N2CC(N3CCN(c4ccc5c(c4)-c4nc(N6C(=O)OC[C@H]6C(F)F)cn4CCO5)CC3)C2)cc1 ; CNC1(C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CCC1]

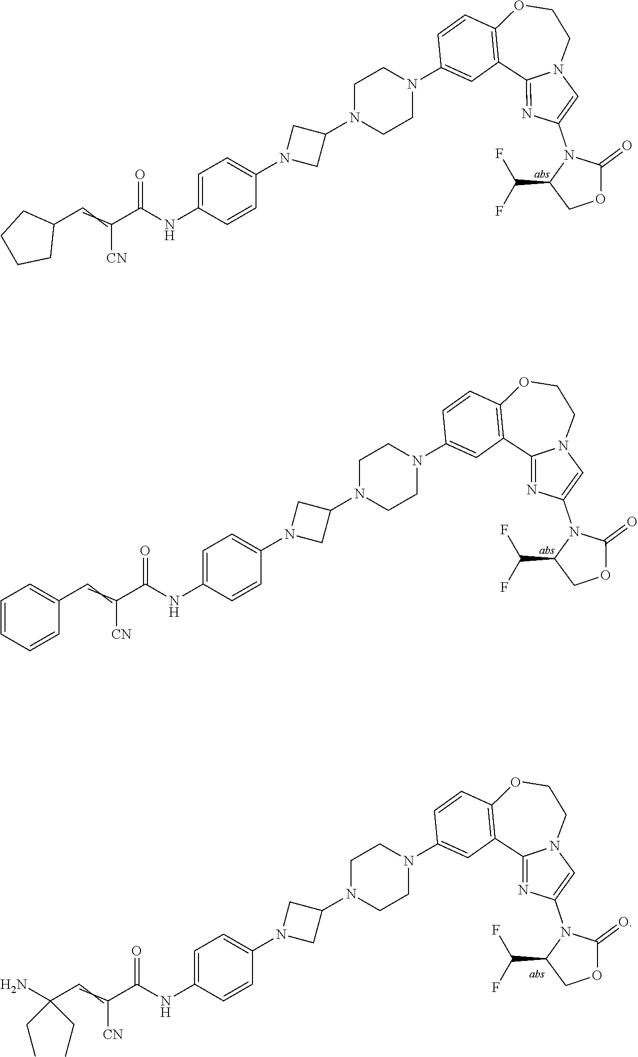 [SMILES: N#CC(=CC1CCCC1)C(=O)Nc1ccc(N2CC(N3CCN(c4ccc5c(c4)-c4nc(N6C(=O)OC[C@H]6C(F)F)cn4CCO5)CC3)C2)cc1 ; N#CC(=Cc1ccccc1)C(=O)Nc1ccc(N2CC(N3CCN(c4ccc5c(c4)-c4nc(N6C(=O)OC[C@H]6C(F)F)cn4CCO5)CC3)C2)cc1 ; CCC(N)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)CC]

Another objective of the present disclosure is to provide a pharmaceutical composition comprising a pharmaceutically acceptable carrier, diluent, and/or excipient, as well as the compound of general formula (1) or the isomer thereof, the crystalline form thereof, the pharmaceutically acceptable salt thereof, the hydrate thereof, or the solvate thereof of the present disclosure as an active ingredient.

Still another objective of the present disclosure provides use of the compound of general formula (1) or the isomer thereof, the crystalline form thereof, the pharmaceutically acceptable salt thereof, the hydrate thereof, or the solvate thereof of the present disclosure, or the pharmaceutical composition described above in preparing a medicament for treating, regulating, or preventing a disease related to PI3Kα. wherein the disease is preferably cancer, and the cancer is a hematologic cancer or a solid tumor.

Still another objective of the present disclosure further provides a method for treating, regulating, or preventing a related disease mediated by PI3Kα, which comprises administering to a subject a therapeutically effective amount of the compound of general formula (1) or the isomer thereof, the crystalline form thereof, the pharmaceutically acceptable salt thereof, the hydrate thereof, or the solvate thereof of the present disclosure, or the pharmaceutical composition described above.

It should be understood that both the aforementioned general description and the following detailed description of the present disclosure are exemplary and explanatory, and are intended to provide further explanation of the present disclosure claimed.

### Synthesis of Compounds

Methods for preparing the compound of general formula (1) of the present disclosure are specifically described below, but these specific methods do not limit the present disclosure in any way.

The compound of general formula (1) described above may be synthesized using standard synthetic techniques or well-known techniques in combination with the methods described herein. In addition, the solvents, temperatures, and other reaction conditions mentioned herein may vary. Starting materials for the synthesis of the compounds may be obtained synthetically or commercially. The compounds described herein and other related compounds with different substituents may be synthesized using well-known techniques and starting materials, including the methods found in March, ADVANCED ORGANIC CHEMISTRY, 4^{th} Ed., (Wiley 1992); Carey and Sundberg, ADVANCED ORGANIC CHEMISTRY, 4^{th} Ed., Vols. A and B (Plenum 2000, 2001); and Green and Wuts, PROTECTIVE GROUPS IN ORGANIC SYNTHESIS, 3^{rd} Ed., (Wiley 1999). General methods for preparing the compounds may be altered by using appropriate reagents and conditions for introducing different groups into the molecular formulas provided herein.

In one aspect, the compounds described herein are prepared according to methods well known in the art. However, the conditions of the methods, such as reactants, solvents, bases, the amount of the compound used, reaction temperature, and time required for the reaction are not limited to the following explanation. The compounds of the present disclosure may also be conveniently prepared by optionally combining various synthetic methods described herein or known in the art, and such combinations may be easily determined by those skilled in the art to which the present disclosure pertains. In one aspect, the present disclosure further provides a method for preparing the compound of general formula (1), which is prepared using general reaction scheme 1 below:

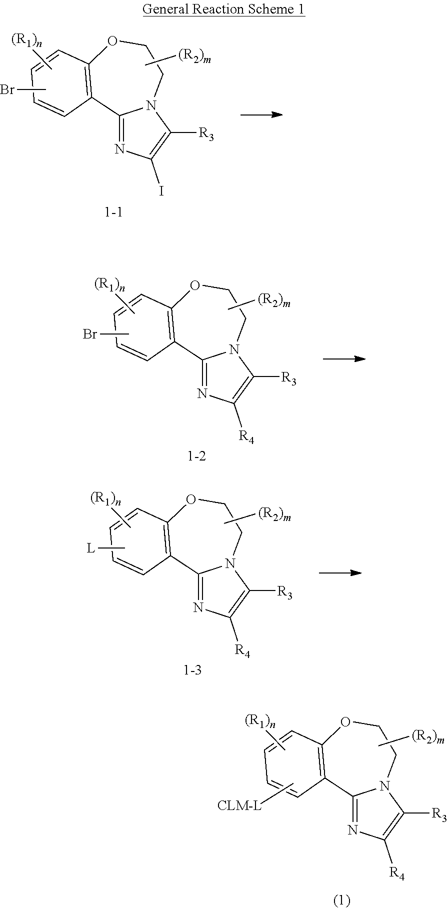

The compound of general formula (1) can be prepared according to general reaction scheme 1, wherein R₁, R₂, R₃, R₄, L, CLM, n, and m are as defined above. As shown in general reaction scheme 1, compound 1-1 is subjected to a coupling reaction with an Rₐ intermediate (NH or boron ester) to generate compound 1-2, compound 1-2 is then sequentially subjected to coupling reactions with each structural unit of L (L₁ to L₅), followed by stepwise structural elongation to give compound 1-3, and finally, compound 1-3 reacts with a corresponding acyl chloride or acid to generate the target general formula compound (1).

### Further Forms of Compounds

“Pharmaceutically acceptable” herein refers to a substance, such as a carrier or diluent, which will not lead to loss of biological activity or properties of a compound and is relatively non-toxic. For example, when an individual is given a substance, the substance will not cause undesired biological effects or interact with any component contained therein in a deleterious manner.

The term “pharmaceutically acceptable salt” refers to a form of a compound that does not cause significant irritation to the organism receiving the administration or eliminate the biological activity and properties of the compound. In certain specific aspects, the pharmaceutically acceptable salt is obtained by subjecting the compound of the general formula to a reaction with acids or bases, wherein the acids or bases include, but are not limited to, those found in Stahl and Wermuth, Handbook of Pharmaceutical Salts: Properties, Selection, and Use, 1^{st} Ed., (Wiley, 2002).

It should be understood that references to pharmaceutically acceptable salts include solvent addition forms or crystalline forms, especially solvates or polymorphs. A solvate contains either stoichiometric or non-stoichiometric amount of solvent and is selectively formed during crystallization in a pharmaceutically acceptable solvent such as water and ethanol. Hydrates are formed when the solvent is water, or alcoholates are formed when the solvent is ethanol. The solvates of the compound of general formula (1) are conveniently prepared or formed according to the methods described herein. For example, hydrates of the compound of general formula (1) are conveniently prepared by recrystallization in a mixed solvent of water/organic solvent, wherein the organic solvent used includes, but is not limited to, tetrahydrofuran, acetone, ethanol, or methanol. Furthermore, the compounds described herein may be present in either a non-solvated form or a solvated form. In general, the solvated forms are considered equivalent to the non-solvated forms for purposes of the compounds and methods provided herein.

In other specific examples, the compound of general formula (1) is prepared in different forms including, but not limited to, amorphous, pulverized, and nanoparticle forms. In addition, the compound of general formula (1) includes crystalline forms, and may also be polymorphs. Polymorphs include different lattice arrangements of the same elements of a compound. Polymorphs generally have different X-ray diffraction spectra, infrared spectra, melting points, density, hardness, crystalline forms, optical and electrical properties, stability, and solubility. Different factors such as a recrystallization solvent, crystallization rate, and storage temperature may lead to a single dominant crystalline form.

In another aspect, the compound of general formula (1) may have a chiral center and/or axial chirality, and thus may be present in the form of a racemate, a racemic mixture, a single enantiomer, a diastereomeric compound, a single diastereomer, and a cis-trans isomer. Each chiral center or axial chirality will independently produce two optical isomers, and all possible optical isomers, diastereomeric mixtures, and pure or partially pure compounds are included within the scope of the present disclosure. The present disclosure is meant to include all such isomeric forms of these compounds.

The compound of the present disclosure may contain unnatural proportions of atomic isotopes at one or more of the atoms that constitute the compound. For example, the compound may be labeled with radioactive isotopes, such as tritium (³H), iodine-125 (¹²⁵I), and C-14 (¹⁴C). For another example, deuterium can be used to substitute a hydrogen atom to form a deuterated compound. The bond formed by deuterium and carbon is stronger than that formed by ordinary hydrogen and carbon. Compared with an undeuterated medicament, the deuterated medicament generally has the advantages of reduced toxic and side effects, increased pharmaceutical stability, enhanced efficacy, prolonged pharmaceutical in vivo half-life, and the like. All isotopic variations of the compound of the present disclosure, whether radioactive or not, are contained within the scope of the present disclosure.

Any atom of the compound of the present disclosure, unless otherwise specified, refers to an isotope of the atom in stable state of the compound. Unless otherwise specified, when a site in a molecular structure is selected as “H” or “hydrogen”, the site should be understood as having the natural abundance of the hydrogen isotope. Similarly, unless otherwise specified, when a site is selected as “D” or “deuterium”, the site should be understood to have a deuterium isotopic abundance that is at least 3000 times its natural abundance (the natural abundance of the deuterium isotope is 0.015%).

More preferably, each deuterated site of the deuterated compounds of the present disclosure has a deuterium atom abundance that is at least 3500 times its natural abundance (52.2% deuterium atom enrichment). More preferably, the deuterium atom abundance is at least 4500 times the natural abundance (67.5% deuterium atom enrichment). More preferably, the deuterium atom abundance is at least 5000 times the natural abundance (75% deuterium atom enrichment). More preferably, the deuterium atom abundance is at least 6000 times the natural abundance (90% deuterium atom enrichment). More preferably, the deuterium atom abundance is at least 6333 times the natural abundance (95% deuterium atom enrichment). More preferably, the deuterium atom abundance is at least 6466.7 times the natural abundance (97% deuterium atom enrichment). More preferably, the deuterium atom abundance is at least 6600 times the natural abundance (99% deuterium atom enrichment). More preferably, the deuterium atom abundance is at least 6633.3 times the natural abundance (99.5% deuterium atom enrichment).

### Terminology

Unless otherwise stated, the terms used in the present application, including those in the specification and claims, are defined as follows. It must be noted that in the specification and the appended claims, the singular forms “a” and “an” include plural meanings unless clearly indicated otherwise. Unless otherwise stated, conventional methods for mass spectrometry, nuclear magnetic resonance spectroscopy, HPLC, protein chemistry, biochemistry, recombinant DNA techniques, and pharmacology are used. As used herein, “or” or “and” refers to “and/or” unless otherwise stated.

Unless otherwise specified, “alkyl” refers to a saturated aliphatic hydrocarbon group, including linear and branched groups containing 1 to 6 carbon atoms. Lower alkyl groups containing 1 to 4 carbon atoms, such as methyl, ethyl, propyl, 2-propyl, n-butyl, isobutyl, and tert-butyl, are preferred. Lower alkyl groups containing 1 to 3 carbon atoms, such as methyl, ethyl, propyl, and 2-propyl, are further preferred. As used herein, “alkyl” includes unsubstituted and substituted alkyl, particularly alkyl substituted with one or more halogens. Preferred alkyl is selected from CH₃, CH₃CH₂, CF₃, CHF₂, CF₃CH₂, CF₃(CH₃)CH, ⁱPr, ⁿPr, ⁱBu, ⁿBu, and ^{t}Bu.

Unless otherwise specified, “alkylene” refers to a divalent alkyl as defined above. Examples of alkylene include, but are not limited to, methylene and ethylene.

Unless otherwise specified, “alkenyl” refers to an unsaturated aliphatic hydrocarbon group containing carbon-carbon double bonds, including linear or branched groups containing 1 to 14 carbon atoms. Lower alkenyl groups containing 1 to 4 carbon atoms, such as vinyl, 1-propenyl, 1-butenyl, or 2-methylpropenyl, are preferred. Lower alkenyl groups containing 1 to 2 carbon atoms are further preferred.

Unless otherwise specified, “alkenylene” refers to a divalent alkenyl as defined above.

Unless otherwise specified, “alkynyl” refers to an unsaturated aliphatic hydrocarbon group containing carbon-carbon triple bonds, including linear and branched groups containing 1 to 14 carbon atoms. Lower alkynyl groups containing 1 to 4 carbon atoms, such as ethynyl, 1-propynyl, or 1-butynyl, are preferred. Lower alkynyl groups containing 1 to 2 carbon atoms are further preferred.

Unless otherwise specified, “alkynylene” refers to a divalent alkynyl as defined above.

Unless otherwise specified, “cycloalkyl” refers to a non-aromatic hydrocarbon ring system (monocyclic, bicyclic, or polycyclic), and is preferably a non-aromatic hydrocarbon ring system containing 3 to 14 ring carbon atoms (C₃₋₁₄ cycloalkyl). In some embodiments, cycloalkyl has 3 to 10 ring carbon atoms (C₃₋₁₀ cycloalkyl). In some embodiments, cycloalkyl has 3 to 8 ring carbon atoms (C₃₋₈ cycloalkyl). In some embodiments, cycloalkyl has 3 to 7 ring carbon atoms (C₃₋₇ cycloalkyl). In some embodiments, cycloalkyl has 3 to 6 ring carbon atoms (C₃₋₆ cycloalkyl). In some embodiments, cycloalkyl has 4 to 6 ring carbon atoms (C₄₋₆ cycloalkyl). In some embodiments, cycloalkyl has 5 to 6 ring carbon atoms (C₅₋₆ cycloalkyl). In some embodiments, cycloalkyl has 5 to 10 ring carbon atoms (C₅₋₁₀ cycloalkyl). For cycloalkyl, partially unsaturated cycloalkyl may be referred to as “cycloalkenyl” if the carbocyclic ring contains at least one double bond, or “cycloalkynyl” if the carbocyclic ring contains at least one triple bond. Cycloalkyl may include monocyclic or polycyclic groups (e.g., having 2, 3, or 4 fused rings) and spiro rings. In some embodiments, cycloalkyl is monocyclic. In some embodiments, cycloalkyl is bicyclic. In some embodiments, cycloalkyl is monocyclic or bicyclic. In some embodiments, cycloalkyl is tricyclic. The ring carbon atoms of cycloalkyl may optionally be oxidized to form an oxo or thio group. Cycloalkyl further includes cycloalkylene. In some embodiments, cycloalkyl contains 0, 1, or 2 double bonds. In some embodiments, cycloalkyl contains 1 or 2 double bonds (partially unsaturated cycloalkyl). In some embodiments, cycloalkyl may be fused to aryl, heteroaryl, cycloalkyl, and heterocycloalkyl. In some embodiments, cycloalkyl may be fused to aryl, cycloalkyl, and heterocycloalkyl. In some embodiments, cycloalkyl may be fused to aryl and heterocycloalkyl. In some embodiments, cycloalkyl may be fused to aryl and cycloalkyl. Examples of cycloalkyl include cyclopropyl, cyclobutyl, cyclopentyl, cyclohexyl, cycloheptyl, cyclopentenyl, cyclohexenyl, cyclohexadienyl, cycloheptatrienyl, norcamphanyl, norpinanyl, norcarnyl, bicyclo[1.1.1]pentyl, bicyclo[2.1.1]hexyl, and the like.

Unless otherwise specified, “cycloalkylene” refers to a divalent cycloalkyl as defined above.

Unless otherwise specified, “alkoxy” refers to an alkyl group that bonds to the rest of the molecule through an ether oxygen atom. Representative alkoxy groups are those having 1 to 6 carbon atoms, such as methoxy, ethoxy, propoxy, isopropoxy, butoxy, isobutoxy, sec-butoxy, and tert-butoxy. As used herein, “alkoxy” includes unsubstituted and substituted alkoxy, particularly alkoxy substituted with one or more halogens. Preferred alkoxy is selected from OCH₃, OCF₃, CHF₂O, CF₃CH₂O, ⁱ⁻PrO, ⁿ⁻PrO, ⁱ⁻BuO, ⁿ⁻BuO, and ^{t-}BuO.

Unless otherwise specified, “aryl” refers to an aromatic hydrocarbon group, which is monocyclic or polycyclic; for example, a monocyclic aryl ring is fused to one or more carbocyclic aromatic groups. Examples of aryl include, but are not limited to, phenyl, naphthyl, and phenanthryl.

Unless otherwise specified, “aryloxy” refers to an aryl group that bonds to the rest of the molecule through an ether oxygen atom. Examples of aryloxy include, but are not limited to, phenoxy and naphthoxy.

Unless otherwise specified, “arylene” refers to a divalent aryl as defined above. Examples of arylene include, but are not limited to, phenylene, naphthylene, and phenanthrylene.

Unless otherwise specified, “heteroaryl” refers to a substituted or unsubstituted aromatic group containing one or more heteroatoms independently selected from O, N, and S, wherein the number of heteroatoms is preferably 1, 2, 3, or 4; preferably, the heteroaryl is a 5- to 14-membered aromatic group containing 1 to 4 heteroatoms selected from oxygen, sulfur, and nitrogen; more preferably, the heteroaryl is a 5- to 9-membered aromatic group containing 1 to 2 heteroatoms optionally selected from oxygen, sulfur, and nitrogen; more preferably, the heteroaryl is a 5- to 6-membered aromatic group containing 1 to 3 heteroatoms optionally selected from oxygen, sulfur, and nitrogen. Heteroaryl is monocyclic or polycyclic. Monocyclic heteroaryl is preferably a 5- to 6-membered aromatic group containing 1 to 3 heteroatoms optionally selected from oxygen, nitrogen, and sulfur. More preferably, monocyclic heteroaryl is a 5- to 6-membered aromatic group containing 1 to 2 heteroatoms optionally selected from oxygen, nitrogen, and sulfur. More preferably, monocyclic heteroaryl is a 5- to 6-membered aromatic group containing 1 heteroatom optionally selected from oxygen, nitrogen, and sulfur. In some embodiments, a monocyclic heteroaryl ring is fused to one or more carbocyclic aromatic groups or other monocyclic heterocycloalkyl groups. Examples of heteroaryl include, but are not limited to, pyridinyl, pyridazinyl, imidazolyl, pyrimidinyl, pyrazolyl, triazolyl, pyrazinyl, quinolinyl, isoquinolinyl, furanyl, thienyl, isoxazolyl, thiazolyl, oxazolyl, 1,2,4-oxadiazolyl, 1,3,4-oxadiazolyl, 1,2,5-oxadiazolyl, isothiazolyl, pyrrolyl, indolyl, benzimidazolyl, benzofuranyl, benzothiazolyl, benzothienyl, benzoxazolyl, benzopyridinyl, pyrrolopyrimidinyl, 1H-pyrrolo[3,2-b]pyridinyl, 1H-pyrrolo[2,3-c]pyridinyl, 1H-pyrrolo[3,2-c]pyridinyl, 1H-pyrrolo[2,3-b]pyridinyl,

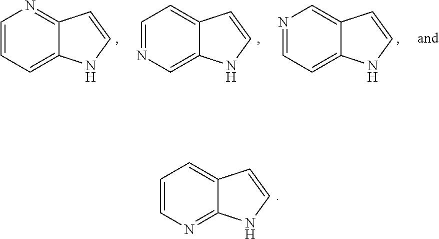

Unless otherwise specified, “heteroarylene” refers to a divalent heteroaryl as defined above.

Unless otherwise specified, “heterocycloalkyl” refers to a non-aromatic ring or ring system, which may optionally contain one or more alkenylene as part of the ring structure, having at least one heteroatom ring member independently selected from boron, phosphorus, nitrogen, sulfur, oxygen, and selenium; heterocycloalkyl is preferably a saturated or partially unsaturated ring containing 1 to 4 heteroatoms selected from oxygen, sulfur, and nitrogen, more preferably a saturated or partially unsaturated ring containing 1 to 2 heteroatoms selected from oxygen, sulfur, and nitrogen. In some embodiments, heterocycloalkyl is a 5- to 8-membered non-aromatic ring containing ring carbon atoms and 1 to 4 ring heteroatoms, and each heteroatom is independently and optionally selected from nitrogen, oxygen, and sulfur (5- to 8-membered heterocycloalkyl). Heterocycloalkyl is a 5- to 6-membered non-aromatic ring containing ring carbon atoms and 1 to 4 ring heteroatoms, and each heteroatom is independently and optionally selected from nitrogen, oxygen, and sulfur (5- to 6-membered heterocycloalkyl). In some embodiments, 5- to 6-membered heterocycloalkyl contains 1 to 3 ring heteroatoms independently selected from nitrogen, oxygen, and sulfur. In some embodiments, 5- to 6-membered heterocycloalkyl contains 1 to 2 ring heteroatoms independently selected from nitrogen, oxygen, and sulfur. In some embodiments, 5- to 6-membered heterocycloalkyl contains 1 ring heteroatom independently selected from nitrogen, oxygen, and sulfur. Partially unsaturated heterocycloalkyl may be referred to as “heterocycloalkenyl” if heterocycloalkyl contains at least one double bond, or “heterocycloalkynyl” if the heterocycloalkyl contains at least one triple bond. Heterocycloalkyl may include monocyclic, bicyclic, spiro ring, or polycyclic (e.g., having two fused or bridged rings) systems. In some embodiments, heterocycloalkyl is a monocyclic group having 1, 2, or 3 heteroatoms independently selected from nitrogen, sulfur, and oxygen. The ring carbon atoms and heteroatoms of heterocycloalkyl may optionally be oxidized to form oxo or thio groups or other oxidized bonds (e.g., C(O), S(O), C(S) or S(O)₂, and N-oxides), or the nitrogen atoms may be quaternized. Heterocycloalkyl may be attached via a ring carbon atom or a ring heteroatom. In some embodiments, heterocycloalkyl contains 0 to 3 double bonds. In some embodiments, heterocycloalkyl contains 0 to 2 double bonds. The definition of heterocycloalkyl further includes moieties (also referred to as partially unsaturated heterocyclic rings) having one or more aromatic rings fused to (i.e., sharing a bond with) the heterocycloalkyl ring, for example, benzo-derivatives of piperidine, morpholine, azepin, and thienyl. Heterocycloalkyl containing a fused aromatic ring may be attached via any ring atom, including ring atoms of the fused aromatic ring. Examples of heterocycloalkyl include, but are not limited to, azetidinyl, azepinyl, dihydrobenzofuranyl, dihydrofuranyl, dihydropyranyl, N-morpholinyl, 3-oxa-9-azaspiro[5.5]undecyl, 1-oxa-8-azaspiro[4.5]decyl, piperidinyl, piperazinyl, oxopiperazinyl, pyranyl, pyrrolidinyl, quininyl, tetrahydrofuranyl, tetrahydropyranyl, 1,2,3,4-tetrahydroquinolinyl, tropanyl, 4,5,6,7-tetrahydrothiazolo[5,4-c]pyridinyl, 4,5,6,7-tetrahydro-1H-imidazo[4,5-c]pyridine, N-methylpiperidinyl, tetrahydroimidazolyl, pyrazolidinyl, butyrolactam, valerolactam, imidazolidinonyl, hydantoinyl, dioxolanyl, phthalimidyl, pyrimidine-2,4(1H,3H)-dione, 1,4-dioxanyl, morpholinyl, thiomorpholinyl, thiomorpholinyl-S-oxide, thiomorpholinyl-S,S-oxide, piperazinyl, pyranyl, pyridonyl, 3-pyrrolinyl, thiopyranyl, pyronyl, tetrahydrothienyl, 2-azaspiro[3.3]heptanyl, indolinyl,

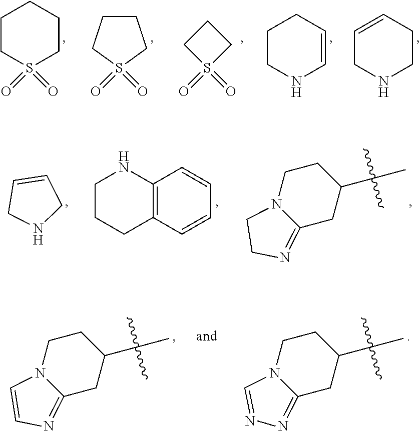

Unless otherwise specified, “heterocycloalkylene” refers to a divalent heterocycloalkyl as defined above. Unless otherwise specified, “heterocyclic spirocycloalkyl”/“spiro heterocycloalkyl”/“spiro heterocyclyl” refers to polycyclic hydrocarbyl formed by sharing one carbon atom (referred to as a spiro atom) between two or more saturated or partially unsaturated monocyclic rings, wherein one or more (e.g., 1, 2 or 3) ring atoms are heteroatoms selected from nitrogen, oxygen, and S(O)ₚ (where p is 0, 1, or 2), and the remaining ring atoms are carbon. When the heteroatom is a nitrogen atom, the nitrogen atom may be substituted or unsubstituted (i.e., N or NR, R being hydrogen or other substituents already defined herein). Each monocyclic ring may contain one or more double bonds, but none of the rings has a fully conjugated π-electron system. According to the number of spiro atoms shared among the rings, the spiro heterocyclyl may be mono-spiro heterocyclyl, di-spiro heterocyclyl, or poly-spiro heterocyclyl. The term “(5- to 15-membered) heterocyclic spirocycloalkyl” refers to a heterocyclic spirocycloalkyl having 5 to 15 ring atoms, wherein the monocyclic ring that shares a spiro atom is a 3- to 8-membered monocyclic ring, and at least 1 monocyclic ring is a heterocycloalkyl ring. Preferred is (6- to 18-membered) heterocyclic spirocycloalkyl having 6 to 18 ring atoms, of which 1-3 ring atoms are heteroatoms. More preferred is (7- to 15-membered) heterocyclic spirocycloalkyl having 7 to 15 ring atoms, of which 1-3 ring atoms are heteroatoms. Most preferred is 9-membered (4-membered monocyclic (heterocyclyl) ring/6-membered monocyclic (heterocyclyl) ring, 5-membered monocyclic (heterocyclyl) ring/5-membered monocyclic (heterocyclyl) ring) mono-spiro heterocyclyl, 10-membered (5-membered monocyclic (heterocyclyl) ring/6-membered monocyclic (heterocyclyl) ring) mono-spiro heterocyclyl, or 11-membered (6-membered monocyclic (heterocyclyl) ring/6-membered monocyclic (heterocyclyl) ring) mono-spiro heterocyclyl. Specific examples of heterocyclic spirocycloalkyl include, but are not limited to,

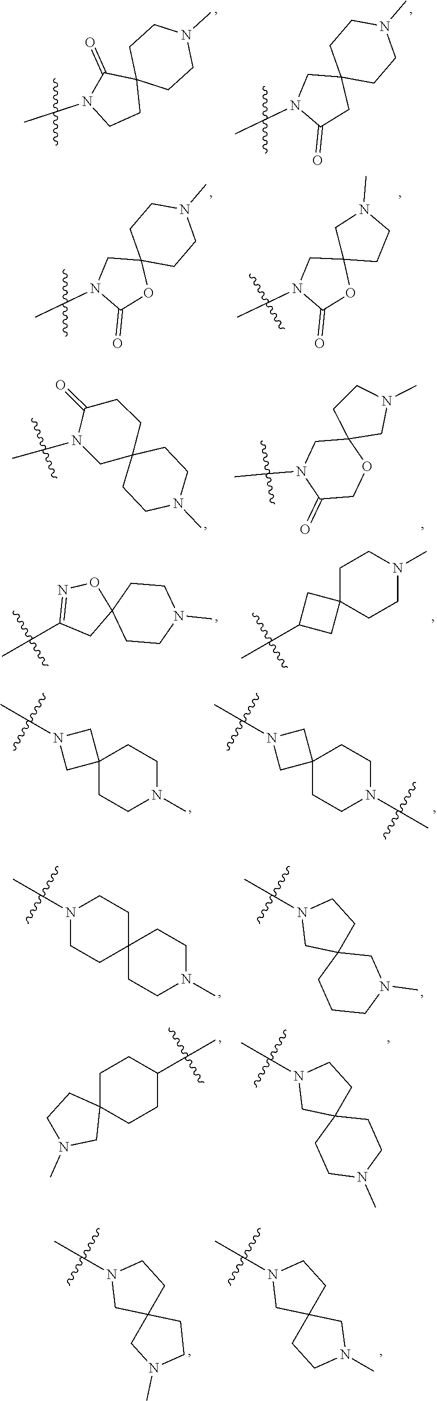

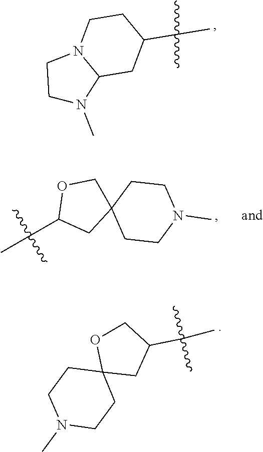

Unless otherwise specified, “bridged heterocycloalkyl”/“bridged heterocyclyl” refers to a 5- to 14-membered polycyclic heterocyclic group, in which any two rings share two non-directly connected atoms, and the group may contain one or more double bonds but has no ring with a fully conjugated π-electron system, wherein one or more ring atoms are heteroatoms selected from nitrogen, oxygen, and S(O)m (where m is an integer of 0 to 2), and the remaining ring atoms are carbon. Preferred is 6- to 14-membered, and more preferred is 7- to 10-membered. According to the number of constituent rings, it may be classified as bicyclic, tricyclic, tetracyclic, or polycyclic bridged heterocyclyl, preferably bicyclic, tricyclic, or tetracyclic, and more preferably bicyclic or tricyclic.

Unless otherwise specified, “oxo” refers to ═O; for example, a group formed by substitution of carbon with one oxo is “carbonyl

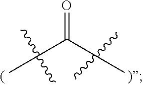 [SMILES: *C(*)=O]

a group formed by substitution of sulfur with one oxo is “sulfinyl

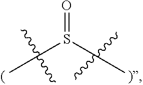 [SMILES: *S(*)=O]

and a group formed by substitution of sulfur with two oxo is “sulfonyl

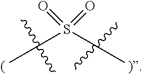 [SMILES: *S(*)(=O)=O]

Unless otherwise specified, “thio” refers to ═S.

Unless otherwise specified, “halogen” (or halo) refers to fluorine, chlorine, bromine, or iodine. The term “halo” (or “halogenated”) before a group name indicates that the group is partially or fully halogenated, that is, substituted in any combination with F, Cl, Br, or I, preferably with F or Cl.

Unless otherwise specified, the term “substituted” means that one or more hydrogen atoms on a designated atom or group are substituted with one or more substituents other than hydrogen without exceeding the normal valence of the designated atom. For example, one or more hydrogens of alkyl, alkylene, alkenyl, alkynyl, hydroxy, amino, or the like may be substituted with one or more substituents. The substituents include, but are not limited to, alkyl, alkenyl, alkynyl, alkoxy, acyl, amino, amido, amidino, aryl, azido, carbamoyl, carboxy, carboxylate, cyano, guanidino, halogen, haloalkyl, heteroalkyl, heteroaryl, heterocyclyl, hydroxy, hydrazino, imino, oxo, nitro, alkylsulfinyl, sulfonic acid, alkylsulfonyl, thiocyanate, thiol, thione, or combinations thereof. The definition of “substituted” does not include analogous indeterminate structures obtained by defining substituents having further substituents attached to infinity (e.g., substituted aryl having substituted alkyl is itself substituted with substituted aryl, which is further substituted with substituted heteroalkyl, and the like). Unless otherwise specified, the maximum number of consecutive substitutions in the compound described herein is three. For example, the consecutive substitutions of substituted aryl with two other substituted aryls are limited to ((substituted aryl)substituted aryl)substituted aryl. Similarly, the definitions described above do not include impermissible substitution patterns (e.g., methyl substituted with 5 fluorines or heteroaryl having two adjacent oxygen ring atoms). Such impermissible substitution patterns are well known to those skilled in the art. When used to modify a chemical group, “substituted” may describe other chemical groups as defined herein. For example, the term “substituted aryl” includes, but is not limited to, “alkylaryl”. Unless otherwise specified, if a group is described as optionally substituted, any substituent of the group is itself unsubstituted. “Optional” or “optionally” means that the subsequently described event or circumstance may, but does not necessarily, occur, and the description includes instances where the event or circumstance occurs and instances where it does not.

Unless otherwise specified, it will be understood that the word “comprise” or variations thereof such as “comprises” or “comprising” refers to the inclusion of a stated element or integer or a group of elements or integers, but not the exclusion of any other element or integer or a group of elements or integers.

The substituent “—O—CH₂—O—” means that two oxygen atoms in the substituent are attached to two adjacent carbon atoms in the heterocycloalkyl, aryl, or heteroaryl, for example:

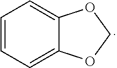

When the number of a linker group is 0, such as —(CH2)₀—, it means that the linker group is a single bond.

When one of the variables is selected from a chemical bond, it means that the two groups attached via this variable are linked directly. For example, when L in X-L-Y represents a chemical bond, it means that the structure is actually X—Y.

The term “membered ring” includes any cyclic structure. The term “membered” is intended to refer to the number of backbone atoms that form a ring. For example, cyclohexyl, pyridinyl, pyranyl, and thiopyranyl are six-membered rings, and cyclopentyl, pyrrolyl, furanyl, and thienyl are five-membered rings.

The term “moiety” refers to a specific portion or functional group of a molecule. A chemical moiety is generally considered to be a chemical entity contained in or attached to a molecule.

The term “isomer” refers to any tautomer, stereoisomer, atropisomer, isotopic isomer, enantiomer, or diastereomer of any compound of the present disclosure. The compound of the present disclosure may have one or more chiral centers or double bonds, and thus exists in the form of stereoisomers, e.g., double bond isomers (i.e., E/Z geometric isomers), or diastereomers (e.g., enantiomers (i.e., (+) or (−)) or cis/trans isomers). The compound of the present disclosure therefore encompasses all corresponding stereoisomers, i.e., stereoisomerically pure (e.g., geometrically pure, enantiomerically pure, or diastereomerically pure) forms, as well as mixtures of enantiomers and stereoisomers, e.g., racemates. The mixtures of enantiomers and stereoisomers of the compound of the present disclosure may be resolved into their component enantiomers or stereoisomers by well-known methods, such as chiral gas chromatography and chiral high-performance liquid chromatography, and by crystallization of the compound in the form of chiral salt complexes or in chiral solvents. Enantiomers and stereoisomers may also be obtained from stereomerically pure or enantiomerically pure intermediates, reagents, and catalysts by well-known asymmetric synthetic methods.

The term “isotopic isomer” refers to distinct molecules that differ in structure only by their isotopic composition and are identical in the remaining structure.

The term “atropisomer” refers to a conformational stereoisomer that results when rotation about a single bond within a molecule is hindered or greatly slowed due to the steric interaction with other parts of the molecule, and the substituents at both ends of the single bond are asymmetric, i.e., the atropisomer does not require a stereocenter. In the case of sufficiently high rotational hindrance around the single bond and sufficiently slow interconversion between conformations, the separation of individual isomers may be allowed (LaPlante et al., J. Med. Chem. 2011, 54, 20, 7005), preferably by a chiral resolution method.

Unless otherwise stated, the absolute configuration of a stereogenic center is represented by a wedged solid bond ( [symbol] ) and a wedged dashed bond ( [symbol] ), and the relative configuration of a stereogenic center is represented by a straight solid bond ( [symbol] ) and a straight dashed bond ( [symbol] ). A wavy line ( [symbol] ) represents a wedged solid bond ( [symbol] ) or a wedged dashed bond ( [symbol] ), or a wavy line ( [symbol] ) represents a straight solid bond ( [symbol] ) or a straight dashed bond ( [symbol] ).

Unless otherwise stated, a single bond or a double bond is represented by [symbol] .

Unless otherwise stated, ( [symbol] ) indicates the E- or Z-configuration of a carbon-carbon double bond, or a mixture of the two. For example,

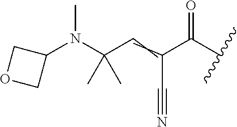 [SMILES: *C(=O)C(C#N)=CC(C)(C)N(C)C1COC1]

represents

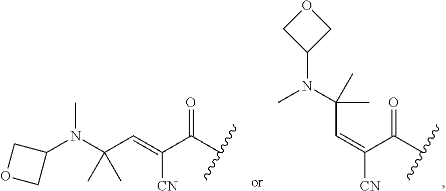 [SMILES: *C(=O)/C(C#N)=C/C(C)(C)N(C)C1COC1 ; *C(=O)/C(C#N)=C\C(C)(C)N(C)C1COC1]

or a mixture of the two.

### Specific Pharmaceutical and Medical Terminology

The term “acceptable”, as used herein, means that a formula component or an active ingredient does not unduly and adversely affect a general therapeutic target's health.

The terms “treatment”, “treatment course”, and “therapy”, as used herein, include alleviating, inhibiting, or ameliorating a symptom or condition of a disease; inhibiting the development of complications; ameliorating or preventing underlying metabolic syndrome; inhibiting the development of a disease or symptom, e.g., controlling the progression of a disease or condition; alleviating a disease or symptom; leading to disease or symptom regression; and alleviating a complication caused by a disease or symptom, or preventing or treating a sign caused by a disease or symptom. As used herein, a compound or pharmaceutical composition, when administered, can ameliorate a disease, symptom, or condition, which particularly refers to ameliorating the severity, delaying the onset, slowing the progression, or reducing the duration of the disease. Fixed or temporary administration, or continuous or intermittent administration, may be attributed to or associated with the administration.

“Active ingredient” refers to the compound of general formula (1), and pharmaceutically acceptable inorganic or organic salts of the compound of general formula (1). The compound of the present disclosure may contain one or more asymmetric centers (chiral center or axial chirality) and thus occurs in the forms of a racemate, a racemic mixture, a single enantiomer, a diastereomeric compound, and a single diastereomer. Asymmetric centers that may be present depend on the nature of the various substituents on the molecule. Each of such asymmetric centers will independently produce two optical isomers, and all possible optical isomers, diastereomeric mixtures and pure or partially pure compounds are included within the scope of the present disclosure. The present disclosure is meant to include all such isomeric forms of these compounds.

The terms such as “compound”, “composition”, “agent”, or “medicine or medicament” are used interchangeably herein and all refer to a compound or composition that, when administered to an individual (human or animal), is capable of inducing a desired pharmacological and/or physiological response by local and/or systemic action.

The term “administered, administering, or administration” refers herein to the direct administration of the compound or composition, or the administration of a prodrug, derivative, analog, or the like of the active compound.

Although the numerical ranges and parameters defining the broad scope of the present disclosure are approximations, the related numerical values set forth in the specific examples have been presented herein as precisely as possible. Any numerical value, however, inherently contains a standard deviation necessarily resulting from certain methods of testing. Herein, “about” generally means that the actual numerical value is within a particular numerical value or range ±10%, 5%, 1%, or 0.5%. Alternatively, the term “about” indicates that the actual numerical value falls within the acceptable standard error of a mean, as considered by those skilled in the art. All ranges, quantities, numerical values, and percentages used herein (e.g., to describe an amount of a material, a length of time, a temperature, an operating condition, a quantitative ratio, and the like) are to be understood as being modified by the word “about”, except in the experimental examples or where otherwise explicitly indicated. Accordingly, unless otherwise contrarily stated, the numerical parameters set forth in the specification and the appended claims are all approximations that may vary as desired. At least, these numerical parameters should be understood as the significant digits indicated or the numerical values obtained using conventional rounding rules.

Unless otherwise defined in the specification, the scientific and technical terms used herein have the same meaning as commonly understood by those skilled in the art. Furthermore, nouns in their singular forms used in the specification encompass their plural forms, unless contradicted by context; nouns in their plural forms used also encompass their singular forms.

### Therapeutic Use

The present disclosure provides a method for treating a disease, including but not limited to a condition involving PI3Kalpha (e.g., cancer), with the compound of general formula (1) or the pharmaceutical composition of the present disclosure.

In some embodiments, a method for treating cancer is provided, the method comprising administering to an individual in need thereof an effective amount of any of the aforementioned pharmaceutical compositions comprising the compound of structural general formula (1). In some embodiments, the cancer is mediated by PI3Kalpha. In other embodiments, the cancer is a hematologic cancer and a solid tumor, including but not limited to, leukemia, breast cancer, lung cancer, pancreatic cancer, colon cancer, bladder cancer, brain cancer, urothelial cancer, prostate cancer, liver cancer, ovarian cancer, head and neck cancer, gastric cancer, mesothelioma, or all cancer metastases.

### Route of Administration

The compound and the pharmaceutically acceptable salt thereof of the present disclosure can be made into various formulations comprising a safe and effective amount of the compound or the pharmaceutically acceptable salt thereof of the present disclosure, and a pharmaceutically acceptable excipient or carrier. The “safe and effective amount” means that the amount of the compound is sufficient to significantly improve the condition without causing serious side effects. The safe and effective amount of the compound is determined according to the age, condition, course of treatment, and other specific conditions of a treated subject.

The “pharmaceutically acceptable excipient or carrier” refers to one or more compatible solid or liquid fillers or gel substances that are suitable for human use and must be of sufficient purity and sufficiently low toxicity. “Compatible” herein means that the components of the composition are capable of intermixing with the compound of the present disclosure and with each other, without significantly diminishing the pharmaceutical efficacy of the compound. Examples of pharmaceutically acceptable excipients or carriers include cellulose and derivatives thereof (e.g., sodium carboxymethylcellulose, sodium ethylcellulose, or cellulose acetate), gelatin, talc, solid lubricants (e.g., stearic acid or magnesium stearate), calcium sulfate, vegetable oil (e.g., soybean oil, sesame oil, peanut oil, or olive oil), polyols (e.g., propylene glycol, glycerol, mannitol, or sorbitol), emulsifiers (e.g., Tween®), wetting agents (e.g., sodium lauryl sulfate), colorants, flavoring agents, stabilizers, antioxidants, preservatives, pyrogen-free water, etc.

When the compound of the present disclosure is administered, it may be administered orally, rectally, parenterally (intravenously, intramuscularly, or subcutaneously), or topically.

Solid dosage forms for oral administration include capsules, tablets, pills, pulvises, and granules. In these solid dosage forms, the active compound is mixed with at least one conventional inert excipient (or carrier), such as sodium citrate or dicalcium phosphate, or with the following ingredients: (a) fillers or extenders, such as starch, lactose, sucrose, glucose, mannitol, and silicic acid; (b) binders, such as hydroxymethyl cellulose, alginate, gelatin, polyvinylpyrrolidone, sucrose, and acacia; (c) humectants, such as glycerol; (d) disintegrants, such as agar, calcium carbonate, potato or tapioca starch, alginic acid, certain complex silicates, and sodium carbonate; (e) solution retarders, such as paraffin; (f) absorption accelerators, such as quaternary ammonium compounds; (g) wetting agents, such as cetyl alcohol and glycerol monostearate; (h) adsorbents, such as kaolin; and (i) lubricants, such as talc, calcium stearate, magnesium stearate, solid polyethylene glycol and sodium lauryl sulfate, or mixtures thereof. In the case of capsules, tablets, and pills, the dosage forms may further include buffers.

Solid dosage forms such as tablets, dragees, capsules, pills, and granules can be prepared using coatings and shells such as enteric coatings and other materials well known in the art. They may include opacifying agents, and the active compound or compound in such a composition may be released in a certain part of the digestive tract in a delayed manner. Examples of embedding components that can be used are polymeric substances and wax-based substances. If necessary, the active compound can also be in microcapsule form with one or more of the excipients described above.

Liquid dosage forms for oral administration include pharmaceutically acceptable emulsions, solutions, suspensions, syrups, and elixirs. In addition to the active compound, the liquid dosage form may include inert diluents commonly used in the art, such as water or other solvents, solubilizers and emulsifiers, for example, ethanol, isopropanol, ethyl carbonate, ethyl acetate, propylene glycol, 1,3-butanediol, dimethylformamide, and oils, especially cottonseed oil, peanut oil, corn germ oil, olive oil, castor oil and sesame oil, or mixtures of these substances.

Besides such inert diluents, the composition may further include adjuvants, such as wetting agents, emulsifiers, suspending agents, sweeteners, flavoring agents, and perfuming agents.

In addition to the active compound, suspensions may include suspending agents, such as ethoxylated isostearyl alcohols, polyoxyethylene sorbitol and sorbitan esters, microcrystalline cellulose, aluminum methylate and agar, or mixtures of these substances.

Compositions for parenteral injection may include physiologically acceptable sterile aqueous or anhydrous solutions, dispersions, suspensions, or emulsions, and sterile powders for redissolving into sterile injectable solutions or dispersions. Suitable aqueous and non-aqueous carriers, diluents, solvents, or excipients include water, ethanol, polyols, and suitable mixtures thereof.

Dosage forms for topical administration of the compound of the present disclosure include ointments, pulvises, patches, sprays, and inhalants. The active ingredient is mixed under sterile conditions with a physiologically acceptable carrier and any preservatives, buffers, or propellants that may be required if necessary.

The compound of the present disclosure may be administered alone or in combination with other pharmaceutically acceptable compounds. When the pharmaceutical composition is used, a safe and effective amount of the compound of the present disclosure is administered to a mammal (such as a human) to be treated, wherein the dose of administration is a pharmaceutically effective dose. For a human of 60 kg, the daily dose of administration is usually 1-2000 mg, preferably 50-1000 mg. In determining a specific dose, such factors as the route of administration, the health condition of the patient, and the like will also be considered, which are well-known to skilled physicians.

The above features mentioned in the present disclosure or those mentioned in the examples may be combined arbitrarily. All the features disclosed in this specification may be used with any composition form and the various features disclosed in this specification may be replaced with any alternative features that provide the same, equivalent, or similar purpose. Thus, unless otherwise specified, the features disclosed herein are merely general examples of equivalent or similar features.

### DETAILED DESCRIPTION

Various specific aspects, features, and advantages of the compounds, methods, and pharmaceutical compositions described above will be set forth in detail in the following description, which will make the content of the present disclosure very clear. It should be understood that the detailed description and examples below describe specific examples for reference only. After reading the description of the present disclosure, those skilled in the art can make various changes or modifications to the present disclosure, and such equivalents also fall within the scope of the present application defined herein.

In all the examples, ¹H-NMR spectra were recorded with a Varian Mercury 400 nuclear magnetic resonance spectrometer, and chemical shifts are represented by δ (ppm); silica gel for separation was 200-300 mesh silica gel if not specified, and the ratio of the eluents was a volume ratio.

### Example 1. Synthesis of Compound 1

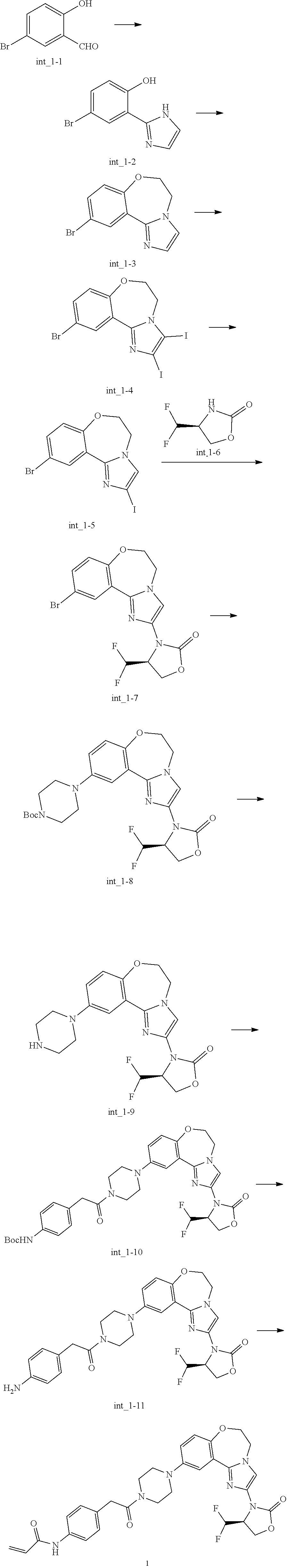

Step 1: Synthesis of Compound int_1-2:

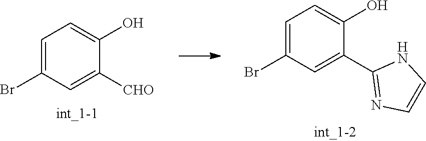 [SMILES: O=Cc1cc(Br)ccc1O ; Oc1ccc(Br)cc1-c1ncc[nH]1]

Compound 5-bromo-2-hydroxybenzaldehyde (20.0 g, 99.5 mmol) was dissolved in methanol (200 mL), followed by addition of glyoxal (72.2 g, 1.24 mol) and ammonia water (89.7 g, 716 mmol). The reaction solution was stirred at 30° C. for 12 h. The reaction solution was filtered, and the filtrate was concentrated under reduced pressure to give crude compound int_1-2 as a black solid.

LCMS m/z (ESI): 238.9 [M+H]⁺.

Step 2: Synthesis of Compound int_1-3:

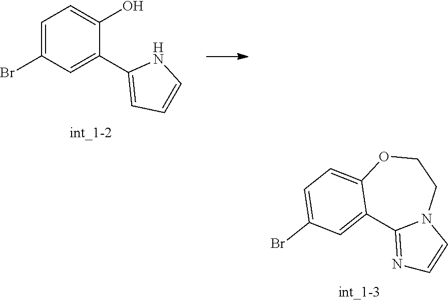 [SMILES: Oc1ccc(Br)cc1-c1ccc[nH]1 ; Brc1ccc2c(c1)-c1nccn1CCO2]

Compound int_1-2 (31.0 g, 130 mmol) was dissolved in N,N-dimethylformamide (300 mL), followed by addition of 1,2-dibromoethane (97.4 g, 519 mmol) and cesium carbonate (169 g, 519 mmol). The reaction solution was stirred at 85° C. for 12 h. The reaction solution was filtered, and the filtrate was concentrated to dryness under reduced pressure. The residue was then separated and purified by silica gel chromatography to give intermediate int_1-3.

LCMS m/z (ESI): 265.1 [M+H]⁺.

Step 3: Synthesis of Compound int_1-4:

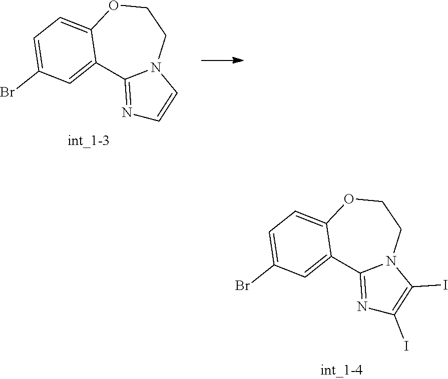 [SMILES: Brc1ccc2c(c1)-c1nccn1CCO2 ; Brc1ccc2c(c1)-c1nc(I)c(I)n1CCO2]

Intermediate int_1-3 (19.0 g, 71.7 mmol) was dissolved in N,N-dimethylformamide (200 mL), followed by addition of N-iodosuccinimide (48.4 g, 215 mmol) at 25° C. The reaction solution was stirred at 60° C. for 12 h. Water (500 mL) was added to the reaction solution, and a large amount of solid precipitated during this process. The mixture was subjected to suction filtration under reduced pressure, and the filter cake was dissolved in ethyl acetate (1.0 L). The resulting mixture was washed with a saturated aqueous sodium hydroxide solution (1.0 L×2) and then with saturated ammonium chloride (1.0 L×2). The organic phases were combined, dried over anhydrous magnesium sulfate, and filtered, and the filtrate was concentrated to dryness under reduced pressure to give crude product int_1-4 as a yellow solid.

LCMS m/z (ESI): 516.9 [M+H]⁺.

¹H NMR (400 MHz, DMSO-d₆) δ 8.36 (d, J=2.6 Hz, 1H), 7.44 (dd, J=2.6, 8.7 Hz, 1H), 7.02-6.96 (m, 1H), 4.49-4.44 (m, 2H), 4.38-4.34 (m, 2H).

Step 4: Synthesis of Compound int_1-5:

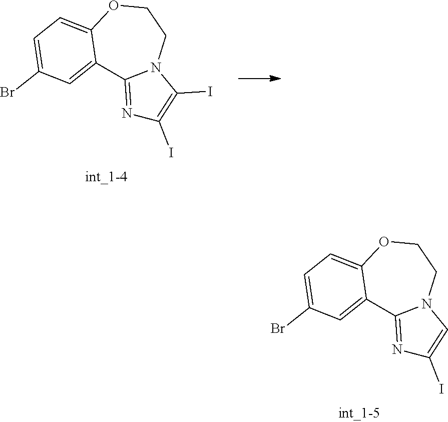 [SMILES: Brc1ccc2c(c1)-c1nc(I)c(I)n1CCO2 ; Brc1ccc2c(c1)-c1nc(I)cn1CCO2]

Compound int_1-4 (28.0 g, 54.2 mmol) was dissolved in anhydrous tetrahydrofuran (280 mL), followed by addition of ethylmagnesium bromide (3 M, 27.1 mL) at −20° C. The reaction solution was stirred at −20° C. for 3 h. The reaction was quenched by adding a saturated aqueous ammonium chloride solution (300 mL) to the reaction solution, and the mixture was extracted with ethyl acetate (300 mL×3). The organic phases were combined, dried over anhydrous magnesium sulfate, and filtered, and the filtrate was concentrated to dryness under reduced pressure. The residue was then separated and purified by silica gel chromatography to give intermediate int_1-5.

LCMS m/z (ESI): 392.1 [M+H]⁺.

¹H NMR (400 MHz, DMSO-d₆) δ 8.38 (d, J=2.5 Hz, 1H), 7.57 (s, 1H), 7.43 (dd, J=2.6, 8.7 Hz, 1H), 6.99 (d, J=8.5 Hz, 1H), 4.43 (s, 4H).

Step 5: Synthesis of Compound int_1-7:

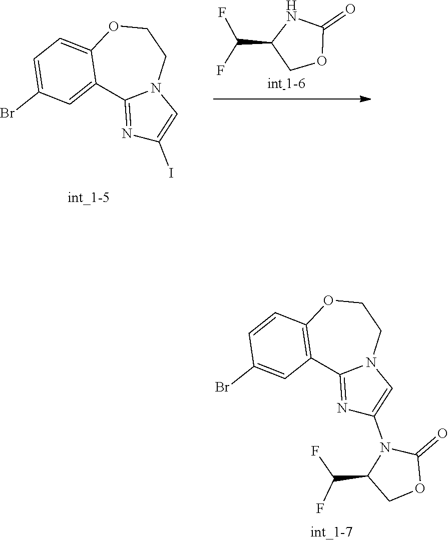 [SMILES: Brc1ccc2c(c1)-c1nc(I)cn1CCO2 ; O=C1N[C@H](C(F)F)CO1 ; O=C1OC[C@@H](C(F)F)N1c1cn2c(n1)-c1cc(Br)ccc1OCC2]

Compound int_1-5 (1.43 g, 3.65 mmol) and (4S)-4-(difluoromethyl)-1,3-oxazolidin-2-one (500 mg, 3.65 mmol) were dissolved in 2-methyltetrahydrofuran (30 mL), followed by addition of copper acetate (132.50 mg, 729.48 μmol), N,N-dimethyl-1,2-cyclohexanediamine (156.06 mg, 1.10 mmol), and cesium carbonate (2.38 g, 7.31 mmol) under nitrogen atmosphere. The reaction solution was stirred at 80° C. for 16 h. After cooling, a saturated aqueous ammonium chloride solution (100 mL) was added to the reaction solution, and the mixed solution was extracted with ethyl acetate (100 mL×3). The organic phases were combined, dried over anhydrous magnesium sulfate, and subjected to suction filtration, and the filtrate was concentrated to dryness under reduced pressure. The residue was then separated and purified by silica gel chromatography to give intermediate int_1-7.

LCMS m/z (ESI): 399.9 [M+H]⁺.

¹H NMR (400 MHz, DMSO-d₆) δ 8.41 (d, J=2.5 Hz, 1H), 7.45-7.39 (m, 2H), 7.00 (d, J=8.8 Hz, 1H), 6.85-6.49 (m, 1H), 5.15-4.97 (m, 1H), 4.66-4.55 (m, 2H), 4.46 (s, 4H).

Step 6: Synthesis of Compound int_1-8:

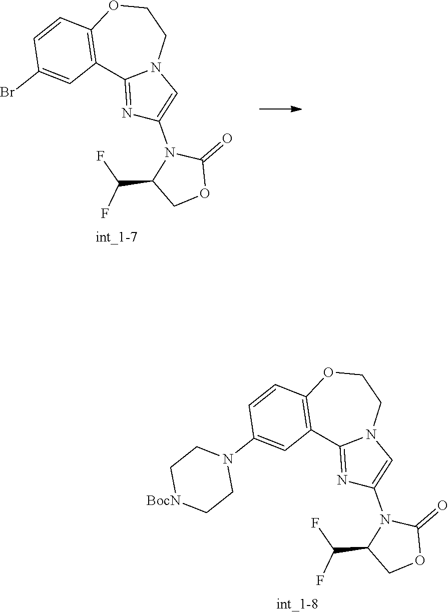 [SMILES: O=C1OC[C@@H](C(F)F)N1c1cn2c(n1)-c1cc(Br)ccc1OCC2 ; CC(C)(C)OC(=O)N1CCN(c2ccc3c(c2)-c2nc(N4C(=O)OC[C@H]4C(F)F)cn2CCO3)CC1]

Compound int_1-7 (300 mg, 749.67 μmol) and N—BOC-piperazine (418.88 mg, 2.25 mmol) were dissolved in tetrahydrofuran (8 mL), followed by addition of cesium carbonate (732.77 mg, 2.25 mmol) and (2-dicyclohexylphosphino-2′,6′-diisopropoxy-1,1′-biphenyl)[2-(2′-amino-1,1′-biphenyl)]palladium(II) methanesulfonate (62.70 mg, 74.97 μmol) under nitrogen atmosphere. The reaction solution was stirred at 80° C. for 16 h. After cooling, water (100 mL) was added to the reaction solution, and the mixed solution was extracted with ethyl acetate (100 mL×3). The organic phases were combined, dried over anhydrous magnesium sulfate, and subjected to suction filtration, and the filtrate was concentrated to dryness under reduced pressure. The residue was then separated and purified by silica gel chromatography to give intermediate int_1-8.

LCMS m/z (ESI): 506.1 [M+H]⁺.

Step 7: Synthesis of Compound int_1-9:

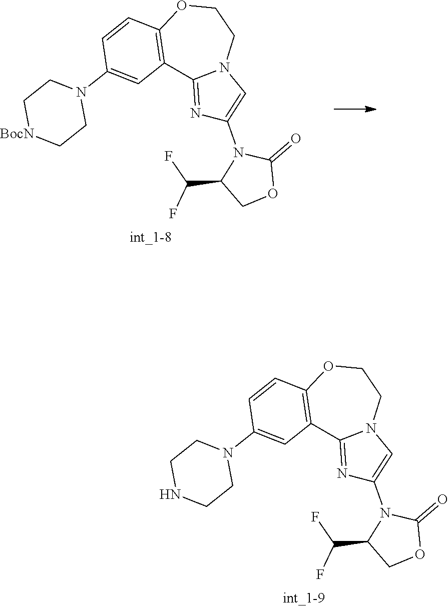 [SMILES: CC(C)(C)OC(=O)N1CCN(c2ccc3c(c2)-c2nc(N4C(=O)OC[C@H]4C(F)F)cn2CCO3)CC1 ; O=C1OC[C@@H](C(F)F)N1c1cn2c(n1)-c1cc(N3CCNCC3)ccc1OCC2]

Compound int_1-8 (330 mg, 652.80 μmol) was dissolved m dichloromethane (12 mL), followed by addition of trifluoroacetic acid (1.5 mL, 15.98 mmol). The reaction solution was stirred at 30° C. for 1 h. N,N-Diisopropylethylamine (2 mL) was slowly added to the reaction solution to adjust the pH to 8 at 0° C., and the mixture was then concentrated to dryness under reduced pressure to give crude product int_1-9 as a yellow solid.

LCMS m/z (ESI): 406.1 [M+H]⁺.

Step 8: Synthesis of Compound int_1-10:

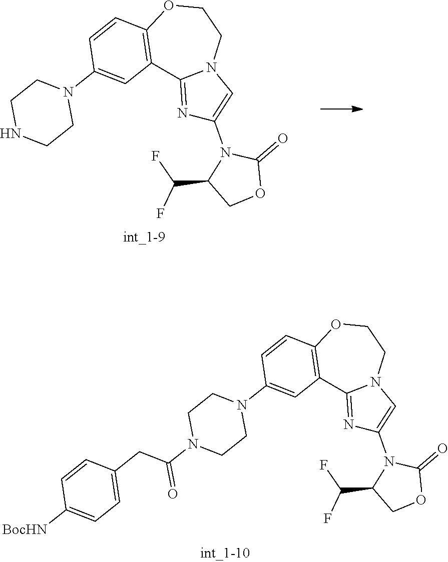 [SMILES: O=C1OC[C@@H](C(F)F)N1c1cn2c(n1)-c1cc(N3CCNCC3)ccc1OCC2 ; CC(C)(C)OC(=O)Nc1ccc(CC(=O)N2CCN(c3ccc4c(c3)-c3nc(N5C(=O)OC[C@H]5C(F)F)cn3CCO4)CC2)cc1]

Compound int_1-9 (80.0 mg, 197 μmol) and 4-Boc-aminophenylacetic acid (59.5 mg, 237 μmol) were dissolved in N,N-dimethylformamide (3 mL), followed by addition of N,N-diisopropylethylamine (76.51 mg, 592 μmol) and O-(7-azabenzotriazol-1-yl)-N,N,N′,N′-tetramethyluronium hexafluorophosphate (90.0 mg, 237 μmol). The reaction solution was stirred at 25° C. for 1 h. The reaction solution was diluted with water (6 mL), and the mixed solution was extracted with ethyl acetate (6 mL×3). The organic phases were combined, dried over anhydrous magnesium sulfate, and subjected to suction filtration, and the filtrate was concentrated to dryness under reduced pressure. The residue was then separated and purified by silica gel chromatography to give intermediate int_1-10.

LCMS m/z (ESI): 639.2 [M+H]⁺.

Step 9: Synthesis of Compound int_1-11:

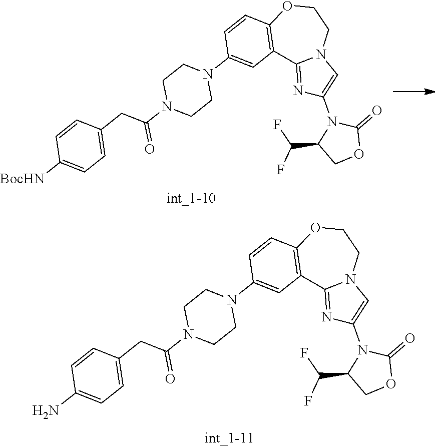 [SMILES: CC(C)(C)OC(=O)Nc1ccc(CC(=O)N2CCN(c3ccc4c(c3)-c3nc(N5C(=O)OC[C@H]5C(F)F)cn3CCO4)CC2)cc1 ; Nc1ccc(CC(=O)N2CCN(c3ccc4c(c3)-c3nc(N5C(=O)OC[C@H]5C(F)F)cn3CCO4)CC2)cc1]

Compound int_1-10 (120 mg, 188 μmol) was dissolved in anhydrous dichloromethane (3 mL), followed by addition of trifluoroacetic acid (643 mg, 5.64 mmol). The reaction solution was stirred at 25° C. for 1 h. The reaction solution was concentrated to dryness under reduced pressure to give crude compound int_1-11 as a brown oil.

LCMS m/z (ESI): 539.1 [M+H]⁺.

### Step 10: Synthesis of Compound 1:

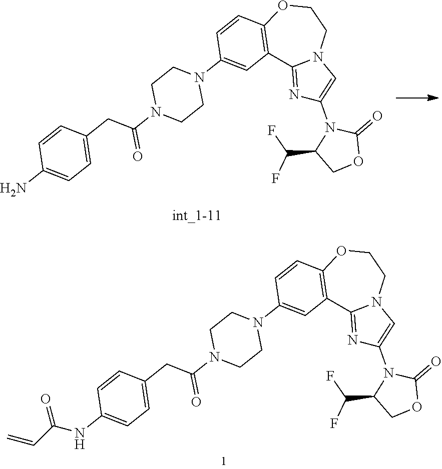 [SMILES: Nc1ccc(CC(=O)N2CCN(c3ccc4c(c3)-c3nc(N5C(=O)OC[C@H]5C(F)F)cn3CCO4)CC2)cc1 ; C=CC(=O)Nc1ccc(CC(=O)N2CCN(c3ccc4c(c3)-c3nc(N5C(=O)OC[C@H]5C(F)F)cn3CCO4)CC2)cc1]

Compound int_1-11 (100 mg, 186 μmol) was dissolved in anhydrous dichloromethane (3 mL), followed by addition of N,N-diisopropylethylamine (56.1 mg, 434 μmol). At 0° C., acryloyl chloride (16.8 mg, 186 μmol) was added. The reaction solution was stirred at 25° C. for 0.2 h. The reaction solution was diluted with a saturated aqueous sodium bicarbonate solution (6 mL), and the mixed solution was extracted with ethyl acetate (6 mL×3). The organic phases were combined, dried over anhydrous magnesium sulfate, and subjected to suction filtration, and the filtrate was concentrated to dryness under reduced pressure. The residue was purified by preparative liquid chromatography to give compound 1 as a white solid.

LCMS m/z (ESI): 593.5 [M+H]⁺.

¹H NMR (400 MHz, DMSO-d₆) δ 10.11 (s, 1H), 7.76 (d, J=2.8 Hz, 1H), 7.59 (d, J=8.5 Hz, 2H), 7.34 (s, 1H), 7.19 (d, J=8.5 Hz, 2H), 7.01-6.88 (m, 2H), 6.85-6.50 (m, 1H), 6.48-6.36 (m, 1H), 6.31-6.18 (m, 1H), 5.78-5.69 (m, 1H), 5.09-4.93 (m, 1H), 4.68-4.52 (m, 2H), 4.42-4.36 (m, 4H), 3.72 (s, 2H), 3.65 (br s, 4H), 3.05-4.97 (m, 4H).

### Example 2. Synthesis of Compound 2

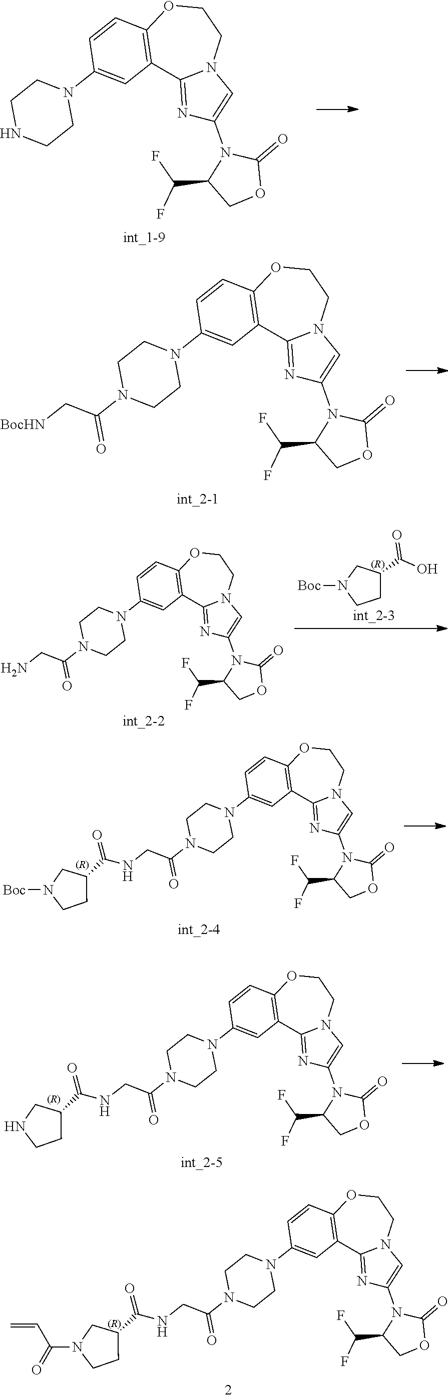

Step 1: Synthesis of Compound int_2-1:

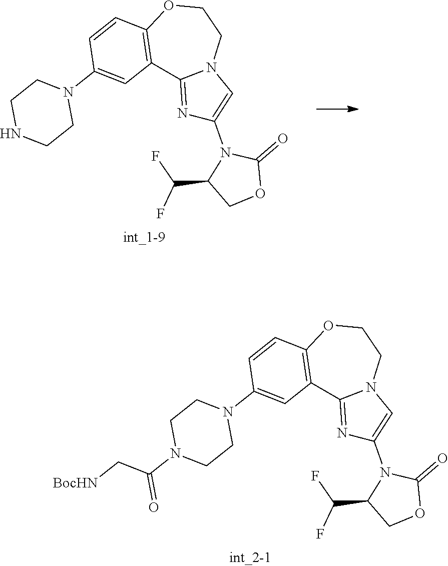 [SMILES: O=C1OC[C@@H](C(F)F)N1c1cn2c(n1)-c1cc(N3CCNCC3)ccc1OCC2 ; CC(C)(C)OC(=O)NCC(=O)N1CCN(c2ccc3c(c2)-c2nc(N4C(=O)OC[C@H]4C(F)F)cn2CCO3)CC1]

Compound int_1-9 (148.30 mg) was dissolved in N,N-dimethylformamide (8 mL), followed by addition of 0-(7-azabenzotriazol-1-yl)-N,N,N′,N′-tetramethyluronium hexafluorophosphate (371.42 mg, 976.82 μmol) and tert-butyl (2-oxo-2-(piperazin-1-yl)ethyl)carbamate (264 mg), and finally, N,N-diisopropylethylamine (907 μL, 5.21 mmol) was added. The reaction solution was stirred at 30° C. for 2 h. Water (100 mL) was added to the reaction solution, and the mixed solution was extracted with ethyl acetate (100 mL×3). The organic phases were combined, dried over anhydrous magnesium sulfate, and subjected to suction filtration, and the filtrate was concentrated to dryness under reduced pressure. The residue was then separated and purified by silica gel chromatography to give intermediate int_2-1.

LCMS m/z (ESI): 563.1 [M+H]⁺.

Step 2: Synthesis of Compound int_2-2:

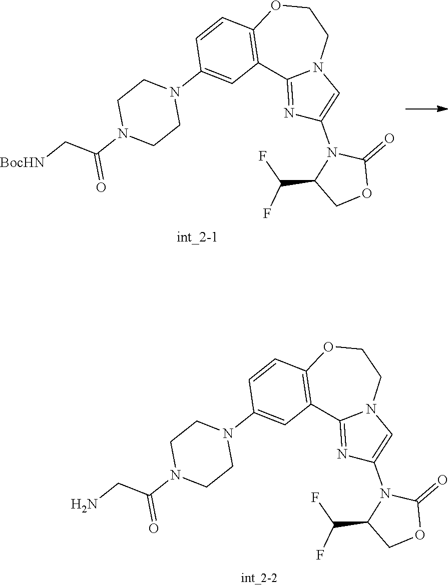 [SMILES: CC(C)(C)OC(=O)NCC(=O)N1CCN(c2ccc3c(c2)-c2nc(N4C(=O)OC[C@H]4C(F)F)cn2CCO3)CC1 ; NCC(=O)N1CCN(c2ccc3c(c2)-c2nc(N4C(=O)OC[C@H]4C(F)F)cn2CCO3)CC1]

Compound int_2-1 (321 mg, 570.60 μmol) was dissolved in dichloromethane (12 mL), followed by addition of trifluoroacetic acid (1.3 mL, 17.12 mmol). The reaction solution was stirred at 30° C. for 1 h. N,N-Diisopropylethylamine (2 mL) was slowly added to the reaction solution to adjust the pH to 8 at 0° C., and the mixture was then concentrated to dryness under reduced pressure to give crude compound int_2-2 as a yellow solid.

LCMS m/z (ESI): 463.1 [M+H]⁺.

Step 3: Synthesis of Compound int_2-4:

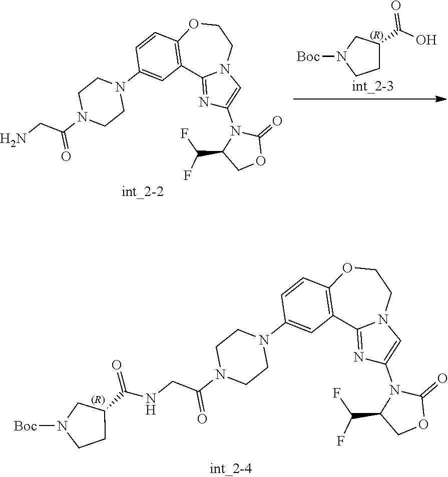

(R)-1-N—BOC-β-proline (27.9 mg, 129.7 μmol) was dissolved in N,N-dimethylformamide (2 mL), followed by addition of O-(7-azabenzotriazol-1-yl)-N,N,N′,N′-tetramethyluronium hexafluorophosphate (61.7 mg, 162.2 μmol), N,N-diisopropylethylamine (41.9 mg, 324.4 μmol), and compound int_2-2 (50.0 mg, 108.1 μmol). The reaction solution was stirred at 25° C. for 2 h. Water (5 mL) was added to the reaction solution, and the mixture was extracted with ethyl acetate (5 mL×3). The organic phases were combined, dried over anhydrous magnesium sulfate, and subjected to suction filtration, and the filtrate was concentrated to dryness under reduced pressure to give crude compound int_2-4 as a yellow oil.

LCMS m/z (ESI): 660.2 [M+H]⁺.

Step 4: Synthesis of Compound int_2-5:

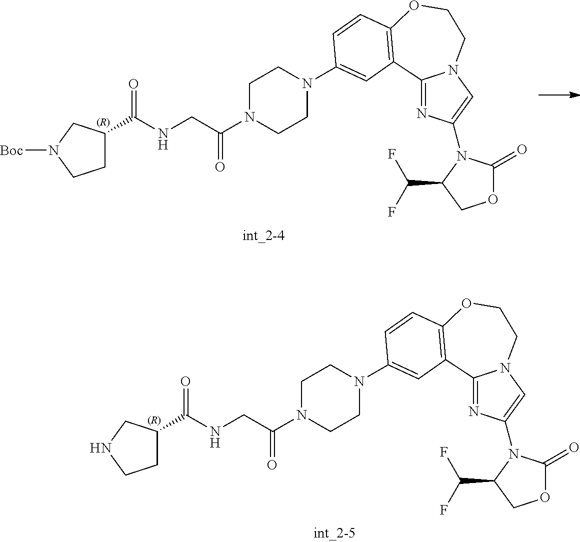

Compound int_2-4 (60.0 mg, 91.0 μmol) was dissolved in dichloromethane (1.5 mL), followed by addition of trifluoroacetic acid (622.3 mg, 5.46 mmol). The reaction solution was stirred at 30° C. for 1 h. The reaction solution was concentrated to dryness under reduced pressure to give compound int_2-5 as a yellow oil.

LCMS m/z (ESI): 560.1 [M+H]⁺.

### Step 5: Synthesis of Compound 2:

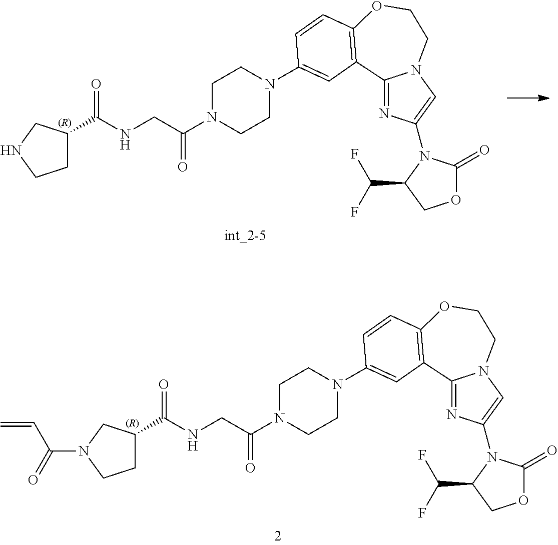

Compound int_2-5 (50.0 mg, 89.4 μmol) was dissolved in dichloromethane (2 mL), followed by addition of N,N-diisopropylethylamine (34.7 mg, 268.1 μmol) and acryloyl chloride (8.1 mg, 89.4 μmol). The reaction solution was shaken with ultrasound at 25° C. for 5 min. A saturated aqueous sodium carbonate solution (5 mL) was added to the reaction solution, and the mixture was extracted with dichloromethane (5 mL×3). The organic phases were combined, dried over anhydrous magnesium sulfate, and subjected to suction filtration, and the filtrate was concentrated to dryness under reduced pressure. The residue was then purified by preparative liquid chromatography to give compound 2 as a white solid.

LCMS m/z (ESI): 614.3 [M+H]⁺.

¹H NMR (400 MHz, DMSO-d₆) δ 8.12-8.28 (m, 1H) 7.79 (d, J=2.76 Hz, 1H) 7.35 (s, 1H) 6.91-7.03 (m, 2H) 6.52-6.85 (m, 2H) 6.08-6.17 (m, 1H) 5.61-5.68 (m, 1H) 4.96-5.09 (m, 1H) 4.51-4.71 (m, 2H) 4.31-4.45 (m, 4H) 4.02 (br d, J=5.52 Hz, 2H) 3.42-3.81 (m, 8H) 3.00-3.15 (m, 5H) 1.88-2.14 (m, 2H).

### Example 3. Synthesis of Compound 3

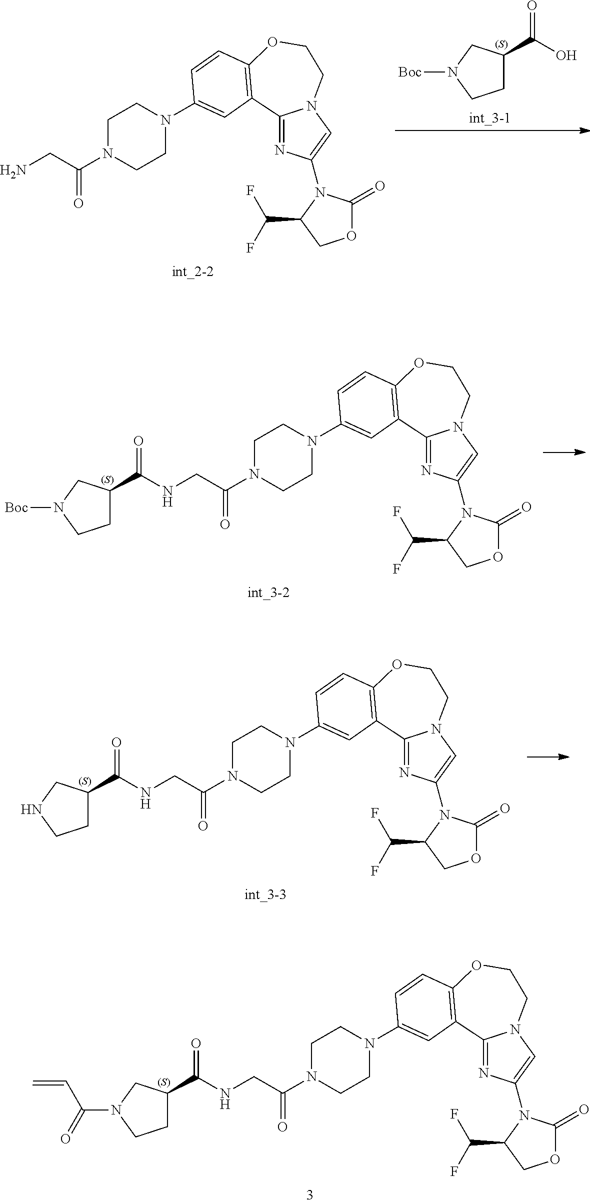

Step 1: Synthesis of Compound int_3-2:

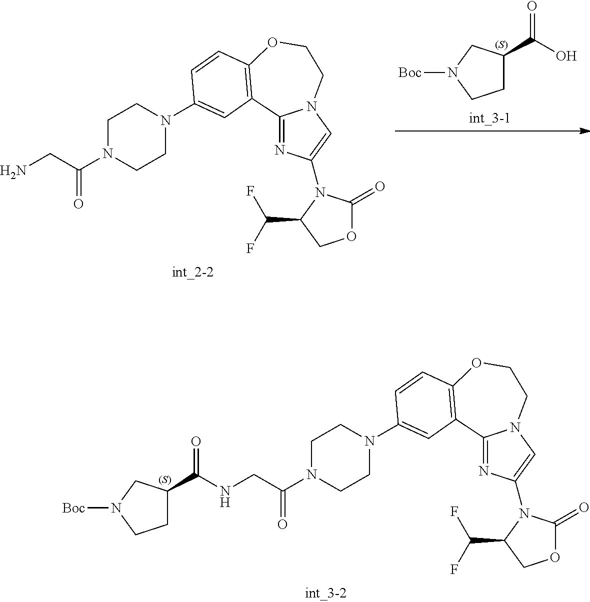

S-1-Boc-pyrrolidine-3-carboxylic acid (22.3 mg, 103.8 μmol) was dissolved in N,N-dimethylformamide (1 mL), followed by addition of O-(7-azabenzotriazol-1-yl)-N,N,N′,N′-tetramethyluronium hexafluorophosphate (49.3 mg, 129.7 μmol), N,N-diisopropylethylamine (33.5 mg, 259.5 μmol), and compound int_2-2 (40.0 mg, 86.5 μmol). The reaction solution was stirred at 25° C. for 2 h. Water (5 mL) was added to the reaction solution, and the mixture was extracted with ethyl acetate (5 mL×3). The organic phases were combined, dried over anhydrous magnesium sulfate, and subjected to suction filtration, and the filtrate was concentrated to dryness under reduced pressure to give crude compound int_3-2 as a yellow oil. LCMS m/z (ESI): 660.2 [M+H]⁺.

Step 2: Synthesis of Compound int_3-3:

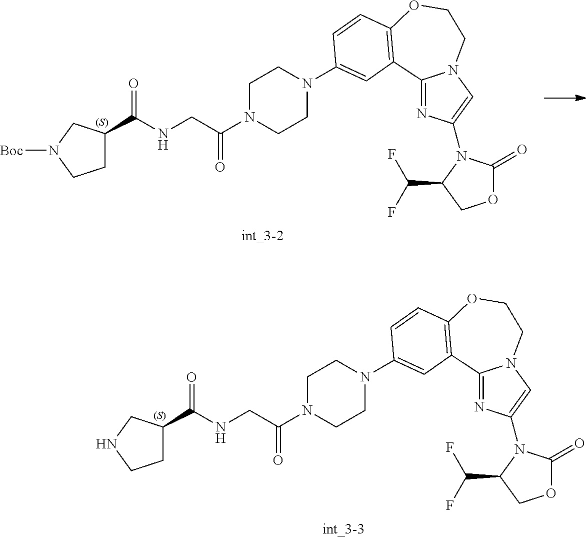

Compound int_3-2 (60.0 mg, 91.0 μmol) was dissolved in dichloromethane (2 mL), followed by addition of trifluoroacetic acid (622.3 mg, 5.46 mmol). The reaction solution was stirred at 30° C. for 1 h. The reaction solution was concentrated under reduced pressure to give crude compound int_3-3 as a yellow oil.

LCMS m/z (ESI): 560.1 [M+H]⁺.

### Step 3: Synthesis of Compound 3:

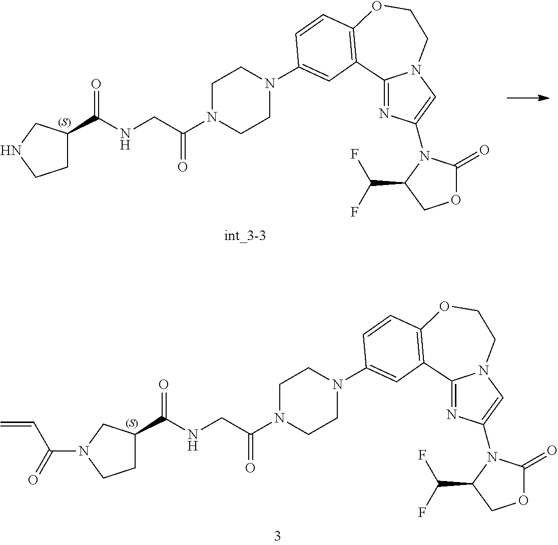

Compound int_3-3 (50.0 mg, 89.4 μmol) was dissolved in dichloromethane (2 mL), followed by addition of N,N-diisopropylethylamine (34.7 mg, 268.1 μmol) and acryloyl chloride (8.1 mg, 89.4 μmol). The reaction solution was shaken with ultrasound at 25° C. for 5 min. A saturated sodium carbonate solution (5 mL) was added to the reaction solution, and the mixture was extracted with dichloromethane (5 mL×3). The organic phases were combined, dried over anhydrous magnesium sulfate, and subjected to suction filtration, and the filtrate was concentrated to dryness under reduced pressure. The residue was then purified by preparative liquid chromatography to give compound 3 as a white solid.

LCMS m/z (ESI): 614.1 [M+H]⁺.

¹H NMR (400 MHz, DMSO-d₆) δ 8.19 (br d, J=2.76 Hz, 1H) 7.79 (d, J=2.51 Hz, 1H) 7.35 (s, 1H) 6.92-7.02 (m, 2H) 6.51-6.83 (m, 2H) 6.12 (br d, J=16.56 Hz, 1H) 5.65 (br d, J=11.29 Hz, 1H) 4.96-5.08 (m, 1H) 4.54-4.67 (m, 2H) 4.35-4.44 (m, 4H) 4.02 (br d, J=5.52 Hz, 2H) 3.47-3.79 (m, 8H) 3.00-3.14 (m, 5H) 1.89-2.12 (m, 2H).

### Example 4. Synthesis of Compound 4

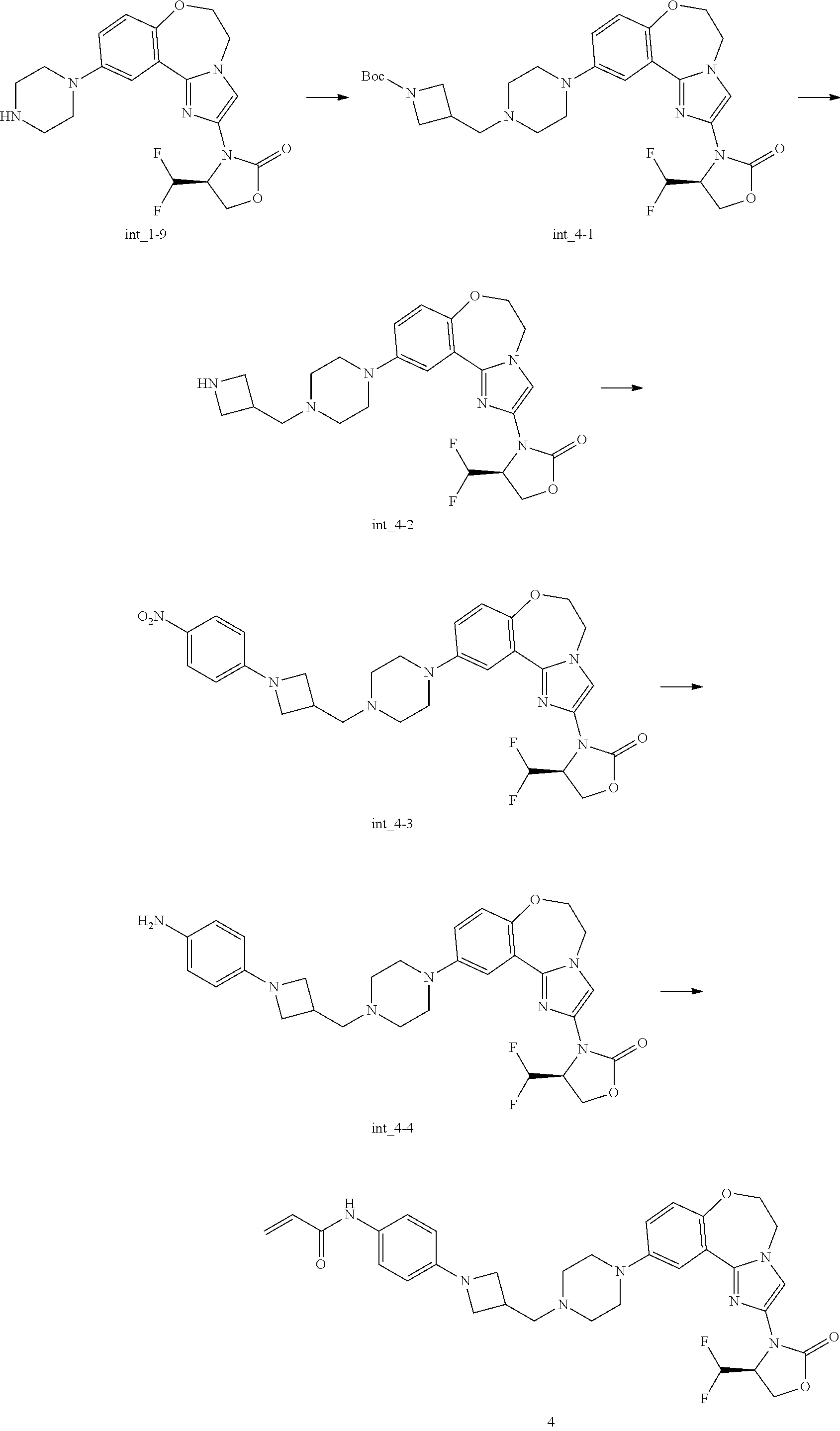 [SMILES: O=C1OC[C@@H](C(F)F)N1c1cn2c(n1)-c1cc(N3CCNCC3)ccc1OCC2 ; CC(C)(C)OC(=O)N1CC(CN2CCN(c3ccc4c(c3)-c3nc(N5C(=O)OC[C@H]5C(F)F)cn3CCO4)CC2)C1 ; O=C1OC[C@@H](C(F)F)N1c1cn2c(n1)-c1cc(N3CCN(CC4CNC4)CC3)ccc1OCC2 ; O=C1OC[C@@H](C(F)F)N1c1cn2c(n1)-c1cc(N3CCN(CC4CN(c5ccc([N+](=O)[O-])cc5)C4)CC3)ccc1OCC2 ; Nc1ccc(N2CC(CN3CCN(c4ccc5c(c4)-c4nc(N6C(=O)OC[C@H]6C(F)F)cn4CCO5)CC3)C2)cc1 ; C=CC(=O)Nc1ccc(N2CC(CN3CCN(c4ccc5c(c4)-c4nc(N6C(=O)OC[C@H]6C(F)F)cn4CCO5)CC3)C2)cc1]

Step 1: Synthesis of Compound int_4-1:

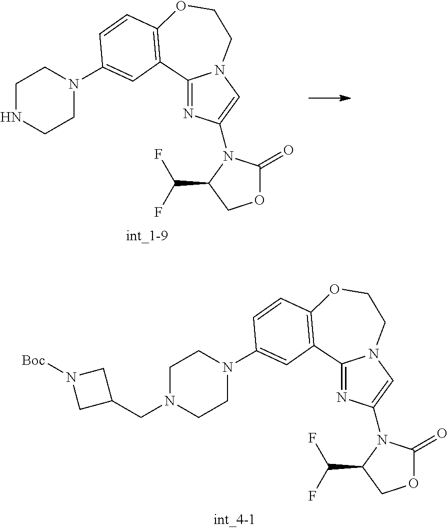 [SMILES: O=C1OC[C@@H](C(F)F)N1c1cn2c(n1)-c1cc(N3CCNCC3)ccc1OCC2 ; CC(C)(C)OC(=O)N1CC(CN2CCN(c3ccc4c(c3)-c3nc(N5C(=O)OC[C@H]5C(F)F)cn3CCO4)CC2)C1]

Compound int_1-9 (200 mg, 493 μmol) and N-Boc-3-formylazetidine (137 mg, 740 μmol) were dissolved in anhydrous tetrahydrofuran (4 mL), followed by addition of triethylamine (150 mg, 1.48 mmol) and sodium triacetoxyborohydride (209 mg, 987 μmol). The reaction solution was stirred at 25° C. for 1 h. The reaction solution was diluted with water (6 mL), and the mixed solution was extracted with ethyl acetate (6 mL×3). The organic phases were combined, dried over anhydrous magnesium sulfate, and subjected to suction filtration, and the filtrate was concentrated to dryness under reduced pressure. The residue was then separated and purified by silica gel chromatography to give intermediate int_4-1.

LCMS m/z (ESI): 575.2 [M+H]⁺.

Step 2: Synthesis of Compound int_4-2:

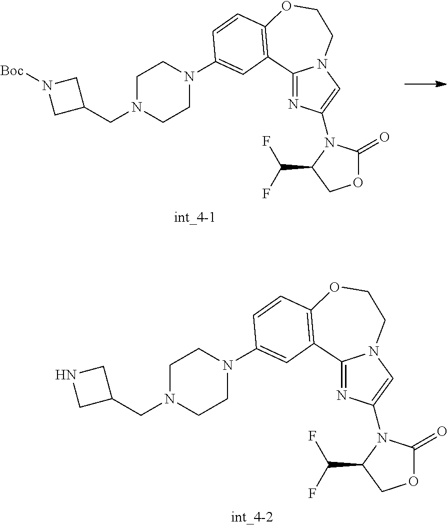 [SMILES: CC(C)(C)OC(=O)N1CC(CN2CCN(c3ccc4c(c3)-c3nc(N5C(=O)OC[C@H]5C(F)F)cn3CCO4)CC2)C1 ; O=C1OC[C@@H](C(F)F)N1c1cn2c(n1)-c1cc(N3CCN(CC4CNC4)CC3)ccc1OCC2]

Compound int_4-1 (230 mg, 400 μmol) was dissolved in anhydrous dichloromethane (3 mL), followed by addition of trifluoroacetic acid (1.37 g, 12.0 mmol). The reaction solution was stirred at 25° C. for 1 h. The reaction solution was concentrated to dryness under reduced pressure to give crude compound int_4-2 as a brown oil.

LCMS m/z (ESI): 475.1 [M+H]⁺.

Step 3: Synthesis of Compound int_4-3:

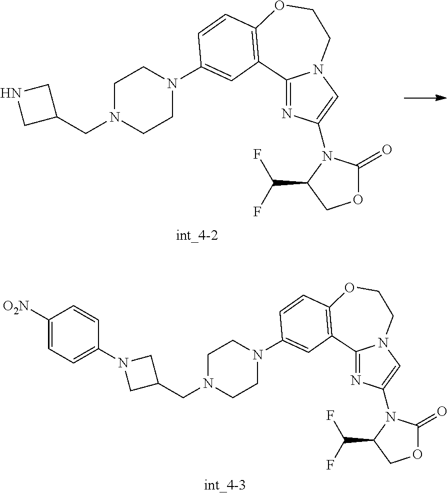 [SMILES: O=C1OC[C@@H](C(F)F)N1c1cn2c(n1)-c1cc(N3CCN(CC4CNC4)CC3)ccc1OCC2 ; O=C1OC[C@@H](C(F)F)N1c1cn2c(n1)-c1cc(N3CCN(CC4CN(c5ccc([N+](=O)[O-])cc5)C4)CC3)ccc1OCC2]

Compound int_4-2 (100 mg, 211 μmol) and 4-fluoronitrobenzene (59.5 mg, 421 μmol) were dissolved in NN-dimethylformamide (3 mL), followed by addition of potassium carbonate (87.4 mg, 632 μmol). The reaction solution was stirred at 100° C. for 1 h. The reaction solution was diluted with water (6 mL), and the mixed solution was extracted with ethyl acetate (6 mL×3). The organic phases were combined, dried over anhydrous magnesium sulfate, and subjected to suction filtration, and the filtrate was concentrated to dryness under reduced pressure. The residue was then separated and purified by silica gel chromatography to give intermediate int_4-3.

LCMS m/z (ESI): 596.1 [M+H]⁺.

Step 4: Synthesis of Compound int_4-4:

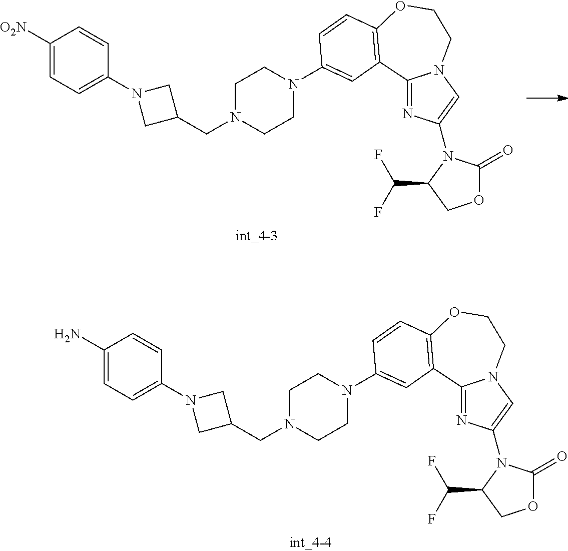 [SMILES: O=C1OC[C@@H](C(F)F)N1c1cn2c(n1)-c1cc(N3CCN(CC4CN(c5ccc([N+](=O)[O-])cc5)C4)CC3)ccc1OCC2 ; Nc1ccc(N2CC(CN3CCN(c4ccc5c(c4)-c4nc(N6C(=O)OC[C@H]6C(F)F)cn4CCO5)CC3)C2)cc1]

Compound int_4-3 (100 mg, 168 μmol) was dissolved in anhydrous ethanol (5 mL) and water (1 mL), followed by addition of ammonium chloride (135 mg, 2.52 mmol) and iron powder (93.8 mg, 1.68 mmol). The reaction solution was stirred at 60° C. for 1 h. The reaction solution was filtered, and the filtrate was concentrated to dryness under reduced pressure. The reaction solution was diluted with a saturated aqueous sodium bicarbonate solution (6 mL), and the mixed solution was extracted with ethyl acetate (6 mL×3). The organic phases were combined, dried over anhydrous magnesium sulfate, and subjected to suction filtration, and the filtrate was concentrated to dryness under reduced pressure to give compound int_4-4 as a yellow solid. LCMS m/z (ESI): 565.1 [M+H]⁺.

### Step: Synthesis of Compound 4:

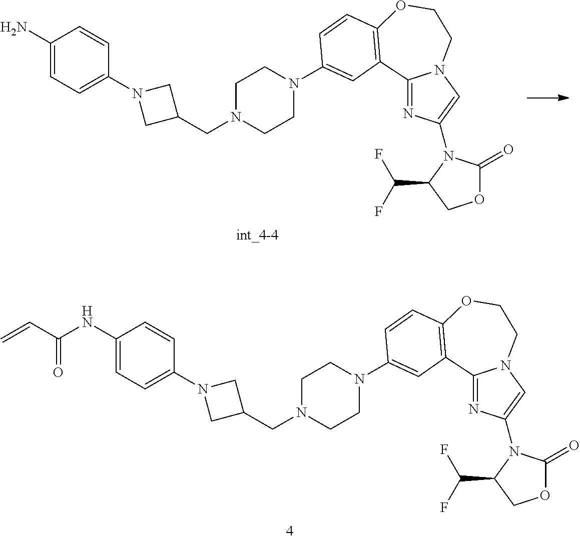 [SMILES: Nc1ccc(N2CC(CN3CCN(c4ccc5c(c4)-c4nc(N6C(=O)OC[C@H]6C(F)F)cn4CCO5)CC3)C2)cc1 ; C=CC(=O)Nc1ccc(N2CC(CN3CCN(c4ccc5c(c4)-c4nc(N6C(=O)OC[C@H]6C(F)F)cn4CCO5)CC3)C2)cc1]

Compound int_4-4 (90 mg, 159 μmol) was dissolved in anhydrous dichloromethane (3 mL), followed by addition of N,N-diisopropylethylamine (61.7 mg, 477 μmol). At 0° C., acryloyl chloride (14.4 mg, 159.1 μmol) was added. The reaction solution was stirred at 25° C. for 0.2 h. The reaction solution was diluted with a saturated aqueous sodium bicarbonate solution (6 mL), and the mixed solution was extracted with ethyl acetate (6 mL×3). The organic phases were combined, dried over anhydrous magnesium sulfate, and subjected to suction filtration, and the filtrate was concentrated to dryness under reduced pressure. The residue was purified by preparative liquid chromatography to give compound 4 as a white solid.

LCMS m/z (ESI): 620.3 [M+H]⁺.

¹H NMR (400 MHz, DMSO-d₆) δ 9.85 (s, 1H), 7.77 (d, J=2.8 Hz, 1H), 7.46 (d, J=8.8 Hz, 2H), 7.34 (s, 1H), 6.98-6.88 (m, 2H), 6.84-6.51 (m, 1H), 6.43-6.33 (m, 3H), 6.23-6.13 (m, 1H), 5.72-5.62 (m, 1H), 5.11-4.96 (m, 1H), 4.66-4.53 (m, 2H), 4.45-4.33 (m, 4H), 3.90 (t, J=7.4 Hz, 2H), 3.49-3.39 (m, 4H), 3.08 (br s, 4H), 2.99-2.88 (m, 1H), 2.69-2.56 (m, 4H).

### Example 5. Synthesis of Compound 5

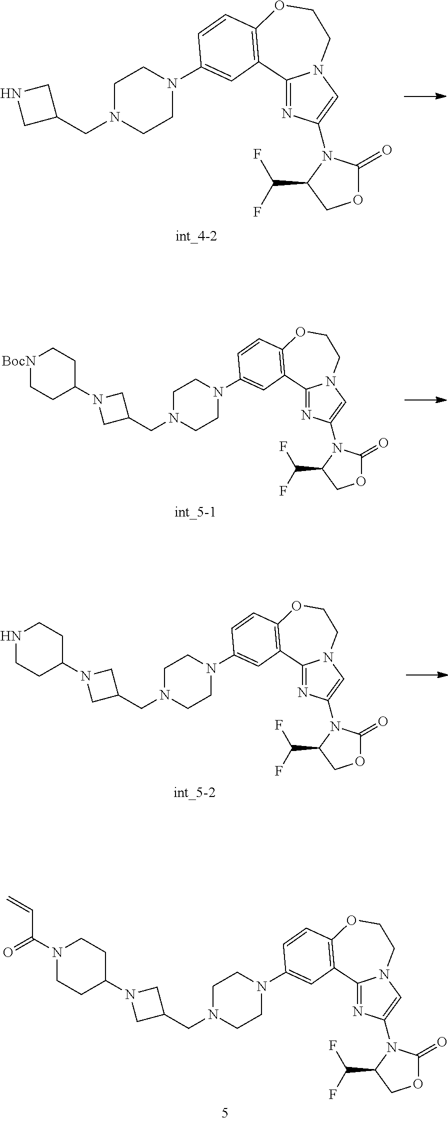 [SMILES: O=C1OC[C@@H](C(F)F)N1c1cn2c(n1)-c1cc(N3CCN(CC4CNC4)CC3)ccc1OCC2 ; CC(C)(C)OC(=O)N1CCC(N2CC(CN3CCN(c4ccc5c(c4)-c4nc(N6C(=O)OC[C@H]6C(F)F)cn4CCO5)CC3)C2)CC1 ; O=C1OC[C@@H](C(F)F)N1c1cn2c(n1)-c1cc(N3CCN(CC4CN(C5CCNCC5)C4)CC3)ccc1OCC2 ; C=CC(=O)N1CCC(N2CC(CN3CCN(c4ccc5c(c4)-c4nc(N6C(=O)OC[C@H]6C(F)F)cn4CCO5)CC3)C2)CC1]

Step 1: Synthesis of Compound int_5-1:

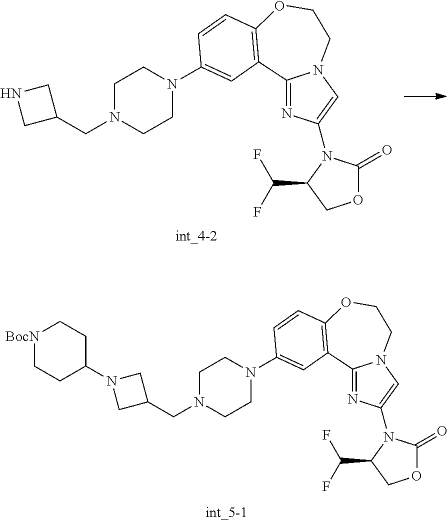 [SMILES: O=C1OC[C@@H](C(F)F)N1c1cn2c(n1)-c1cc(N3CCN(CC4CNC4)CC3)ccc1OCC2 ; CC(C)(C)OC(=O)N1CCC(N2CC(CN3CCN(c4ccc5c(c4)-c4nc(N6C(=O)OC[C@H]6C(F)F)cn4CCO5)CC3)C2)CC1]

Compound int_4-2 (100 mg, 211 μmol) and N-tert-butoxycarbonyl-4-piperidone (63.0 mg, 316 μmol) were dissolved in anhydrous tetrahydrofuran (3 mL), followed by addition of N,N-diisopropylethylamine (81.7 mg, 632 μmol) and sodium triacetoxyborohydride (89.3 mg, 422 μmol). The reaction solution was stirred at 25° C. for 1 h. The reaction solution was diluted with water (6 mL), and the mixed solution was extracted with ethyl acetate (6 mL×3). The organic phases were combined, dried over anhydrous magnesium sulfate, and subjected to suction filtration, and the filtrate was concentrated to dryness under reduced pressure. The residue was then separated and purified by silica gel chromatography to give intermediate int_5-1. LCMS m/z (ESI): 658.3 [M+H]⁺.

Step 2: Synthesis of Compound int_5-2:

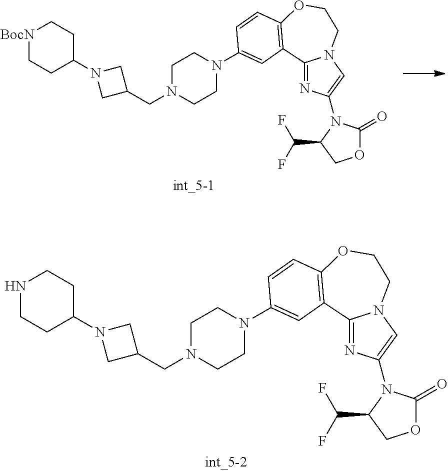 [SMILES: CC(C)(C)OC(=O)N1CCC(N2CC(CN3CCN(c4ccc5c(c4)-c4nc(N6C(=O)OC[C@H]6C(F)F)cn4CCO5)CC3)C2)CC1 ; O=C1OC[C@@H](C(F)F)N1c1cn2c(n1)-c1cc(N3CCN(CC4CN(C5CCNCC5)C4)CC3)ccc1OCC2]

Compound int_5-1 (130 mg, 198 μmol) was dissolved in anhydrous dichloromethane (3 mL), followed by addition of trifluoroacetic acid (451 mg, 3.95 mmol). The reaction solution was stirred at 25° C. for 1 h. The reaction solution was concentrated to dryness under reduced pressure to give crude compound int_5-2 as a brown oil.

LCMS m/z (ESI): 558.2 [M+H]⁺.

### Step 3: Synthesis of Compound 5:

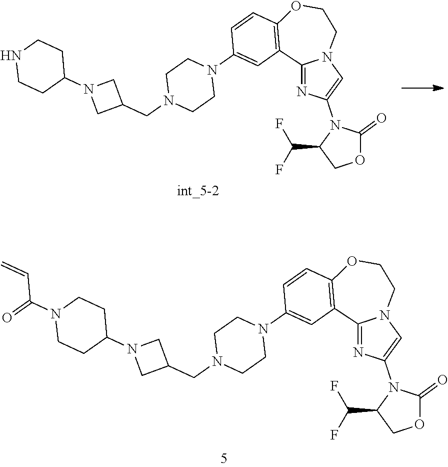 [SMILES: O=C1OC[C@@H](C(F)F)N1c1cn2c(n1)-c1cc(N3CCN(CC4CN(C5CCNCC5)C4)CC3)ccc1OCC2 ; C=CC(=O)N1CCC(N2CC(CN3CCN(c4ccc5c(c4)-c4nc(N6C(=O)OC[C@H]6C(F)F)cn4CCO5)CC3)C2)CC1]

Compound int_5-2 (110 mg, 197 μmol) was dissolved in anhydrous dichloromethane (3 mL), followed by addition of N,N-diisopropylethylamine (76.5 mg, 592 μmol). At 0° C., acryloyl chloride (17.9 mg, 197 μmol) was added. The reaction solution was stirred at 25° C. for 0.2 h. The reaction solution was diluted with a saturated aqueous sodium bicarbonate solution (6 mL), and the mixed solution was extracted with ethyl acetate (6 mL×3). The organic phases were combined, dried over anhydrous magnesium sulfate, and subjected to suction filtration, and the filtrate was concentrated to dryness under reduced pressure. The residue was purified by preparative liquid chromatography to give compound 5 as a white solid.

LCMS m/z (ESI): 612.3 [M+H]⁺.

¹H NMR (400 MHz, DMSO-d₆) δ 7.75 (d, J=2.5 Hz, 1H), 7.34 (s, 1H), 6.96-6.88 (m, 2H), 6.82-6.50 (m, 2H), 6.05 (dd, J=2.4, 16.7 Hz, 1H), 5.66-5.60 (m, 1H), 5.09-4.95 (m, 1H), 4.66-4.53 (m, 2H), 4.43-4.33 (m, 4H), 4.04-3.75 (m, 2H), 3.34-3.29 (m, 7H), 3.22-2.90 (m, 6H), 2.73 (br t, J=6.4 Hz, 2H), 2.59-2.53 (m, 2H), 2.21 (br s, 1H), 1.59 (br s, 2H), 1.05 (br s, 2H).

### Example 6. Synthesis of Compound 6

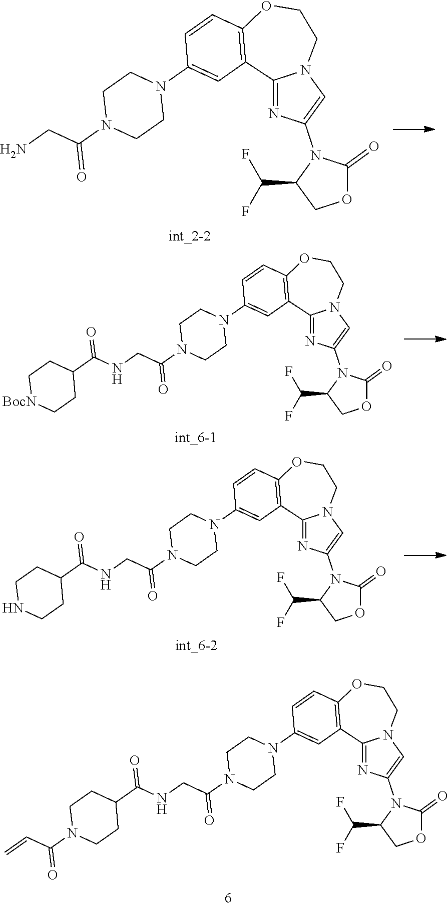 [SMILES: NCC(=O)N1CCN(c2ccc3c(c2)-c2nc(N4C(=O)OC[C@H]4C(F)F)cn2CCO3)CC1 ; CC(C)(C)OC(=O)N1CCC(C(=O)NCC(=O)N2CCN(c3ccc4c(c3)-c3nc(N5C(=O)OC[C@H]5C(F)F)cn3CCO4)CC2)CC1 ; O=C(NCC(=O)N1CCN(c2ccc3c(c2)-c2nc(N4C(=O)OC[C@H]4C(F)F)cn2CCO3)CC1)C1CCNCC1 ; C=CC(=O)N1CCC(C(=O)NCC(=O)N2CCN(c3ccc4c(c3)-c3nc(N5C(=O)OC[C@H]5C(F)F)cn3CCO4)CC2)CC1]

Step 1: Synthesis of Compound int_6-1:

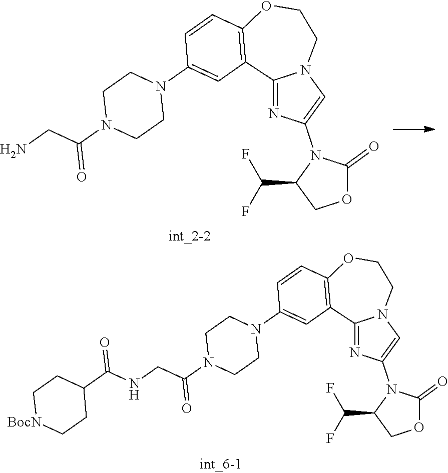 [SMILES: NCC(=O)N1CCN(c2ccc3c(c2)-c2nc(N4C(=O)OC[C@H]4C(F)F)cn2CCO3)CC1 ; CC(C)(C)OC(=O)N1CCC(C(=O)NCC(=O)N2CCN(c3ccc4c(c3)-c3nc(N5C(=O)OC[C@H]5C(F)F)cn3CCO4)CC2)CC1]

1-Boc-4-piperidinecarboxylic acid (169.51 mg, 739.32 μmol) was dissolved in N,N-dimethylformamide (5 mL), followed by addition of O-(7-azabenzotriazol-1-yl)-N,N,N′,N′-tetramethyluronium hexafluorophosphate (324.36 mg, 853.07 μmol) and compound int_2-2 (263 mg, 568.71 μmol), and finally, N,N-diisopropylethylamine (792.47 μL, 5.21 mmol) was added. The reaction solution was stirred at 30° C. for 2 h. Water (100 mL) was added to the reaction solution, and the mixed solution was extracted with ethyl acetate (100 mL×3). The organic phases were combined, dried over anhydrous magnesium sulfate, and subjected to suction filtration, and the filtrate was concentrated to dryness under reduced pressure. The residue was then separated and purified by silica gel chromatography to give intermediate int_6-1.

LCMS m/z (ESI): 674.2 [M+H]⁺.

Step 2: Synthesis of Compound int_6-2:

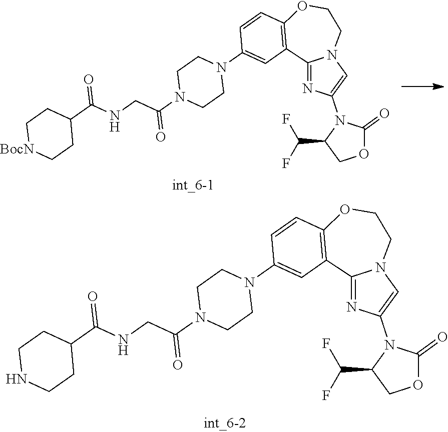 [SMILES: CC(C)(C)OC(=O)N1CCC(C(=O)NCC(=O)N2CCN(c3ccc4c(c3)-c3nc(N5C(=O)OC[C@H]5C(F)F)cn3CCO4)CC2)CC1 ; O=C(NCC(=O)N1CCN(c2ccc3c(c2)-c2nc(N4C(=O)OC[C@H]4C(F)F)cn2CCO3)CC1)C1CCNCC1]

Compound int_6-1 (170 mg, 252.34 μmol) was dissolved in dichloromethane (5 mL), followed by addition of trifluoroacetic acid (561 μL, 7.57 mmol). The reaction solution was stirred at 30° C. for 2 h. N,N-Diisopropylethylamine (2 mL) was slowly added to the reaction solution to adjust the pH to 8 at 0° C., and the mixture was then concentrated to dryness under reduced pressure to give crude compound int_6-2 as a yellow solid.

LCMS m/z (ESI): 574.2 [M+H]⁺.

### Step 3: Synthes of Compound 6:

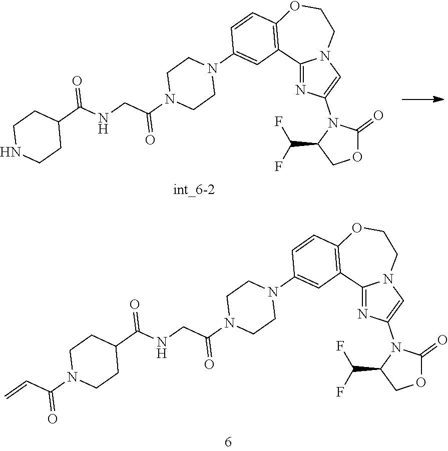 [SMILES: O=C(NCC(=O)N1CCN(c2ccc3c(c2)-c2nc(N4C(=O)OC[C@H]4C(F)F)cn2CCO3)CC1)C1CCNCC1 ; C=CC(=O)N1CCC(C(=O)NCC(=O)N2CCN(c3ccc4c(c3)-c3nc(N5C(=O)OC[C@H]5C(F)F)cn3CCO4)CC2)CC1]

Compound int_6-2 (145 mg, 252.79 μmol) was dissolved in dichloromethane (6 mL), followed by addition of N,N-diisopropylethylamine (132 μL, 758 μmol). Then, acryloyl chloride (21 μL, 253 μmol) was added at 0° C., and the reaction solution was stirred at 30° C. for 5 min. A saturated aqueous sodium carbonate solution (15 mL) was added to the reaction solution, and the mixed solution was extracted with dichloromethane (15 mL×3). The organic phases were combined, dried over anhydrous magnesium sulfate, and subjected to suction filtration, and the filtrate was concentrated to dryness under reduced pressure. The residue was purified by preparative liquid chromatography to give compound 6 as a white solid.

LCMS m/z (ESI): 628.2 [M+H]⁺.

¹H NMR (400 MHz, DMSO-d₆) δ 7.98 (t, J=5.4 Hz, 1H), 7.79 (d, J=2.8 Hz, 1H), 7.35 (s, 1H), 7.02-6.90 (m, 2H), 6.85-6.75 (m, 1H), 6.73-6.48 (m, 1H), 6.20-6.01 (m, 1H), 5.75-5.57 (m, 1H), 5.10-4.94 (m, 1H), 4.67-4.51 (m, 2H), 4.46-4.31 (m, 5H), 4.09-3.93 (m, 3H), 3.67-3.53 (m, 4H), 3.17-2.98 (m, 5H), 2.77-2.65 (m, 1H), 2.58-2.54 (m, 1H), 1.81-1.56 (m, 2H), 1.59-1.32 (m, 2H).

### Example 7. Synthesis of Compound 7

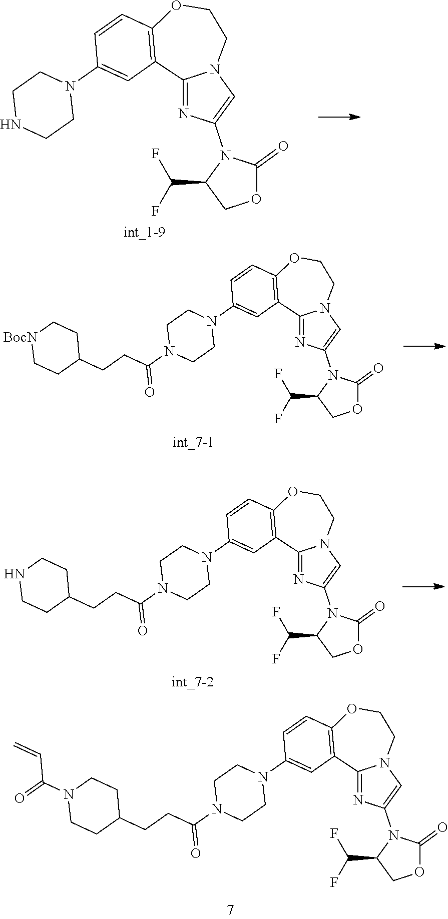 [SMILES: O=C1OC[C@@H](C(F)F)N1c1cn2c(n1)-c1cc(N3CCNCC3)ccc1OCC2 ; CC(C)(C)OC(=O)N1CCC(CCC(=O)N2CCN(c3ccc4c(c3)-c3nc(N5C(=O)OC[C@H]5C(F)F)cn3CCO4)CC2)CC1 ; O=C(CCC1CCNCC1)N1CCN(c2ccc3c(c2)-c2nc(N4C(=O)OC[C@H]4C(F)F)cn2CCO3)CC1 ; C=CC(=O)N1CCC(CCC(=O)N2CCN(c3ccc4c(c3)-c3nc(N5C(=O)OC[C@H]5C(F)F)cn3CCO4)CC2)CC1]

### Step 1: Synthesis of Compound Int_7-1:

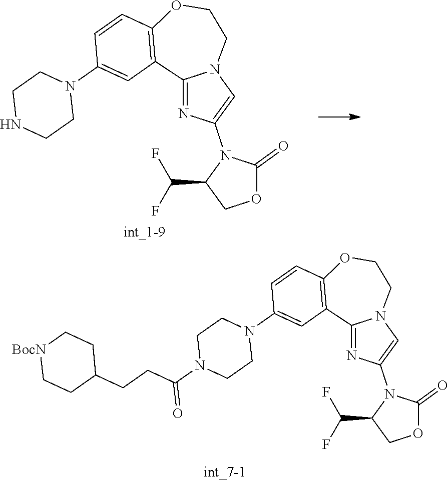 [SMILES: O=C1OC[C@@H](C(F)F)N1c1cn2c(n1)-c1cc(N3CCNCC3)ccc1OCC2 ; CC(C)(C)OC(=O)N1CCC(CCC(=O)N2CCN(c3ccc4c(c3)-c3nc(N5C(=O)OC[C@H]5C(F)F)cn3CCO4)CC2)CC1]

1-N—BOC-4-piperidinepropionic acid (67.03 mg, 260.48 μmol) was dissolved in N,N-dimethylformamide (3 mL), followed by addition of O-(7-azabenzotriazol-1-yl)-N,N,N′,N′-tetramethyluronium hexafluorophosphate (123.81 mg, 325.61 μmol) and compound int_1-9 (88 mg), and finally, N,N-diisopropylethylamine (113 μL, 651 μmol) was added. The reaction solution was stirred at 30° C. for 2 h. Water (30 mL) was added to the reaction solution, and the mixed solution was extracted with ethyl acetate (30 mL×3). The organic phases were combined, dried over anhydrous magnesium sulfate, and subjected to suction filtration, and the filtrate was concentrated to dryness under reduced pressure. The residue was then separated and purified by silica gel chromatography to give intermediate int_7-1.

LCMS m/z (ESI): 645.2 [M+H]⁺.

Step 2: Synthesis of Compound int_7-2:

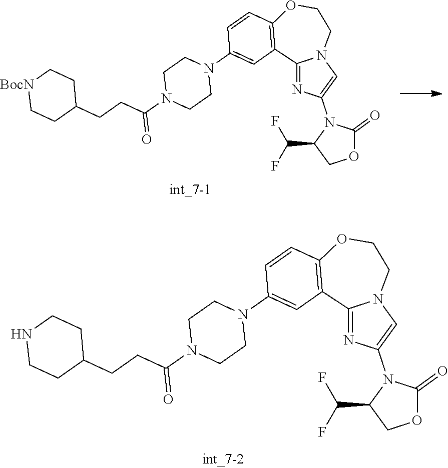 [SMILES: CC(C)(C)OC(=O)N1CCC(CCC(=O)N2CCN(c3ccc4c(c3)-c3nc(N5C(=O)OC[C@H]5C(F)F)cn3CCO4)CC2)CC1 ; O=C(CCC1CCNCC1)N1CCN(c2ccc3c(c2)-c2nc(N4C(=O)OC[C@H]4C(F)F)cn2CCO3)CC1]

Compound int_7-1 (120 mg, 186.13 μmol) was dissolved in dichloromethane (4 mL), followed by addition of trifluoroacetic acid (413 μL, 5.58 mmol). The reaction solution was stirred at 30° C. for 2 h. N,N-Diisopropylethylamine (2 mL) was slowly added to the reaction solution to adjust the pH to 8 at 0° C., and the mixture was then concentrated to dryness under reduced pressure to give crude compound int_7-2 as a yellow solid.

LCMS m/z (ESI): 545.2 [M+H]⁺.

### Step 3: Synthesis of Compound 7:

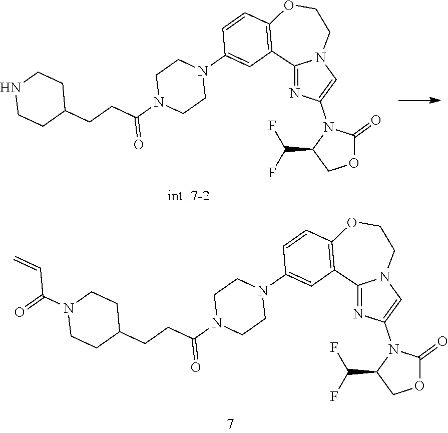 [SMILES: O=C(CCC1CCNCC1)N1CCN(c2ccc3c(c2)-c2nc(N4C(=O)OC[C@H]4C(F)F)cn2CCO3)CC1 ; C=CC(=O)N1CCC(CCC(=O)N2CCN(c3ccc4c(c3)-c3nc(N5C(=O)OC[C@H]5C(F)F)cn3CCO4)CC2)CC1]

Compound int_7-2 (100 mg, 183.62 μmol) was dissolved in dichloromethane (3 mL), followed by addition of N,N-diisopropylethylamine (160 μL, 918 μmol). Then, acryloyl chloride (7 μL, 90 μmol) was added at 0° C., and the reaction solution was stirred at 30° C. for 5 min. A saturated aqueous sodium carbonate solution (15 mL) was added to the reaction solution, and the mixed solution was extracted with dichloromethane (15 mL×3). The organic phases were combined, dried over anhydrous magnesium sulfate, and subjected to suction filtration, and the filtrate was concentrated to dryness under reduced pressure. The residue was purified by preparative liquid chromatography to give compound 7 as a white solid.

LCMS m/z (ESI): 599.2 [M+H]⁺.

¹H NMR (400 MHz, DMSO-d₆) δ 7.79 (d, J=2.8 Hz, 1H), 7.35 (s, 1H), 7.07-6.90 (m, 2H), 6.85-6.77 (m, 1H), 6.76-6.51 (m, 1H), 6.20-6.01 (m, 1H), 5.70-5.63 (m, 1H), 5.12-4.93 (m, 1H), 4.68-4.53 (m, 2H), 4.48-4.31 (m, 5H), 4.02 (br d, J=13.7 Hz, 1H), 3.69-3.55 (m, 4H), 3.15-2.92 (m, 5H), 2.43-2.35 (m, 3H), 1.72 (br d, J=12.5 Hz, 2H), 1.58-1.39 (m, 3H), 1.10-0.90 (m, 2H).

### Example 8. Synthesis of Compound 11

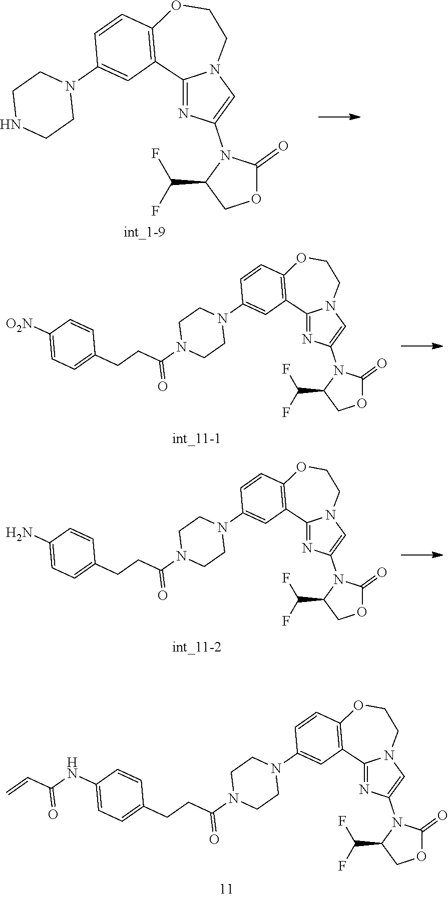 [SMILES: O=C1OC[C@@H](C(F)F)N1c1cn2c(n1)-c1cc(N3CCNCC3)ccc1OCC2 ; O=C(CCc1ccc([N+](=O)[O-])cc1)N1CCN(c2ccc3c(c2)-c2nc(N4C(=O)OC[C@H]4C(F)F)cn2CCO3)CC1 ; Nc1ccc(CCC(=O)N2CCN(c3ccc4c(c3)-c3nc(N5C(=O)OC[C@H]5C(F)F)cn3CCO4)CC2)cc1 ; C=CC(=O)Nc1ccc(CCC(=O)N2CCN(c3ccc4c(c3)-c3nc(N5C(=O)OC[C@H]5C(F)F)cn3CCO4)CC2)cc1]

Step 1: Synthesis of Compound int_11-1:

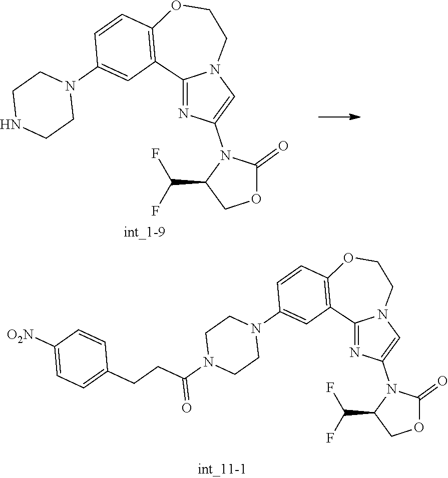 [SMILES: O=C1OC[C@@H](C(F)F)N1c1cn2c(n1)-c1cc(N3CCNCC3)ccc1OCC2 ; O=C(CCc1ccc([N+](=O)[O-])cc1)N1CCN(c2ccc3c(c2)-c2nc(N4C(=O)OC[C@H]4C(F)F)cn2CCO3)CC1]

Compound int_1-9 (80.0 mg, 197 μmol) and 4-nitrobenzoic acid (46.2 mg, 237 μmol) were dissolved in N,N-dimethylformamide (3 mL), followed by addition of N,N-diisopropylethylamine (51.0 mg, 395 μmol) and 0-(7-azabenzotriazol-1-yl)-N,N,N′,N′-tetramethyluronium hexafluorophosphate (90.0 mg, 237 μmol). The reaction solution was stirred at 25° C. for 2 h. The reaction solution was diluted with water (6 mL), and the mixed solution was extracted with ethyl acetate (6 mL×3). The organic phases were combined, dried over anhydrous magnesium sulfate, and subjected to suction filtration, and the filtrate was concentrated to dryness under reduced pressure. The residue was then separated and purified by silica gel chromatography to give intermediate int_11-1.

LCMS m/z (ESI): 583.1 [M+H]⁺.

Step 2: Synthesis of Compound int_11-2:

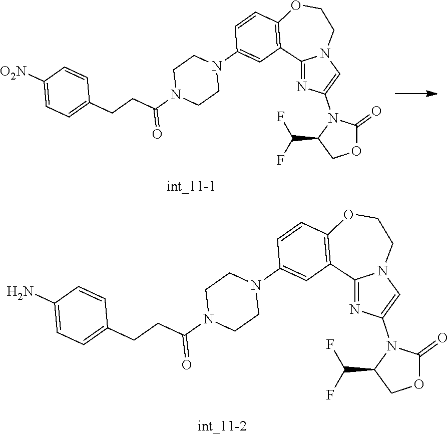 [SMILES: O=C(CCc1ccc([N+](=O)[O-])cc1)N1CCN(c2ccc3c(c2)-c2nc(N4C(=O)OC[C@H]4C(F)F)cn2CCO3)CC1 ; Nc1ccc(CCC(=O)N2CCN(c3ccc4c(c3)-c3nc(N5C(=O)OC[C@H]5C(F)F)cn3CCO4)CC2)cc1]

Compound int_11-1 (110 mg, 189 μmol) was dissolved in anhydrous ethanol (3 mL) and water (0.5 mL), followed by addition of ammonium chloride (152 mg, 2.83 mmol) and iron powder (158 mg, 2.83 mmol). The reaction solution was stirred at 60° C. for 1 h. After cooling, the reaction solution was filtered, and the filtrate was concentrated to dryness under reduced pressure. The reaction solution was diluted with a saturated aqueous sodium bicarbonate solution (6 mL), and the mixed solution was extracted with ethyl acetate (6 mL×3). The organic phases were combined, dried over anhydrous magnesium sulfate, and subjected to suction filtration, and the filtrate was concentrated to dryness under reduced pressure to give crude compound int_11-2 as a yellow solid.

LCMS m/z (ESI): 553.1 [M+H]⁺.

### Step 3: Synthesis of Compound 11:

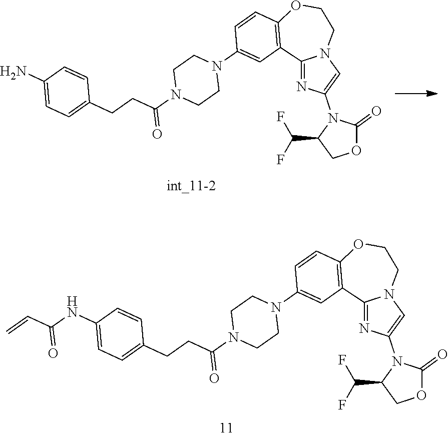 [SMILES: Nc1ccc(CCC(=O)N2CCN(c3ccc4c(c3)-c3nc(N5C(=O)OC[C@H]5C(F)F)cn3CCO4)CC2)cc1 ; C=CC(=O)Nc1ccc(CCC(=O)N2CCN(c3ccc4c(c3)-c3nc(N5C(=O)OC[C@H]5C(F)F)cn3CCO4)CC2)cc1]

Compound int_11-2 (100 mg, 181 μmol) was dissolved in anhydrous dichloromethane (2 mL), followed by addition of N,N-diisopropylethylamine (56.1 mg, 434 μmol). At 0° C., acryloyl chloride (16.4 mg, 181 μmol) was added. The reaction solution was stirred at 25° C. for 0.2 h. The reaction solution was diluted with a saturated aqueous sodium bicarbonate solution (6 mL), and the mixed solution was extracted with ethyl acetate (6 mL×3). The organic phases were combined, dried over anhydrous magnesium sulfate, and subjected to suction filtration, and the filtrate was concentrated to dryness under reduced pressure. The residue was purified by preparative liquid chromatography to give compound 11 as a white solid.

LCMS m/z (ESI): 607.2 [M+H]⁺.

¹H NMR (400 MHz, DMSO-d₆) δ 10.08 (br s, 1H), 7.83 (br s, 1H), 7.57 (d, J=8.3 Hz, 2H), 7.36 (s, 1H), 7.21 (d, J=8.3 Hz, 2H), 7.05-6.92 (m, 2H), 6.87-6.53 (m, 1H), 6.49-6.36 (m, 1H), 6.32-6.19 (m, 1H), 5.79-5.68 (m, 1H), 5.11-4.94 (m, 1H), 4.69-4.54 (m, 2H), 4.47-4.34 (m, 4H), 3.61 (br s, 4H), 3.04 (br s, 4H), 2.86-2.76 (m, 2H), 2.71-2.62 (m, 2H).

### Example 9. Synthesis of Compound 12

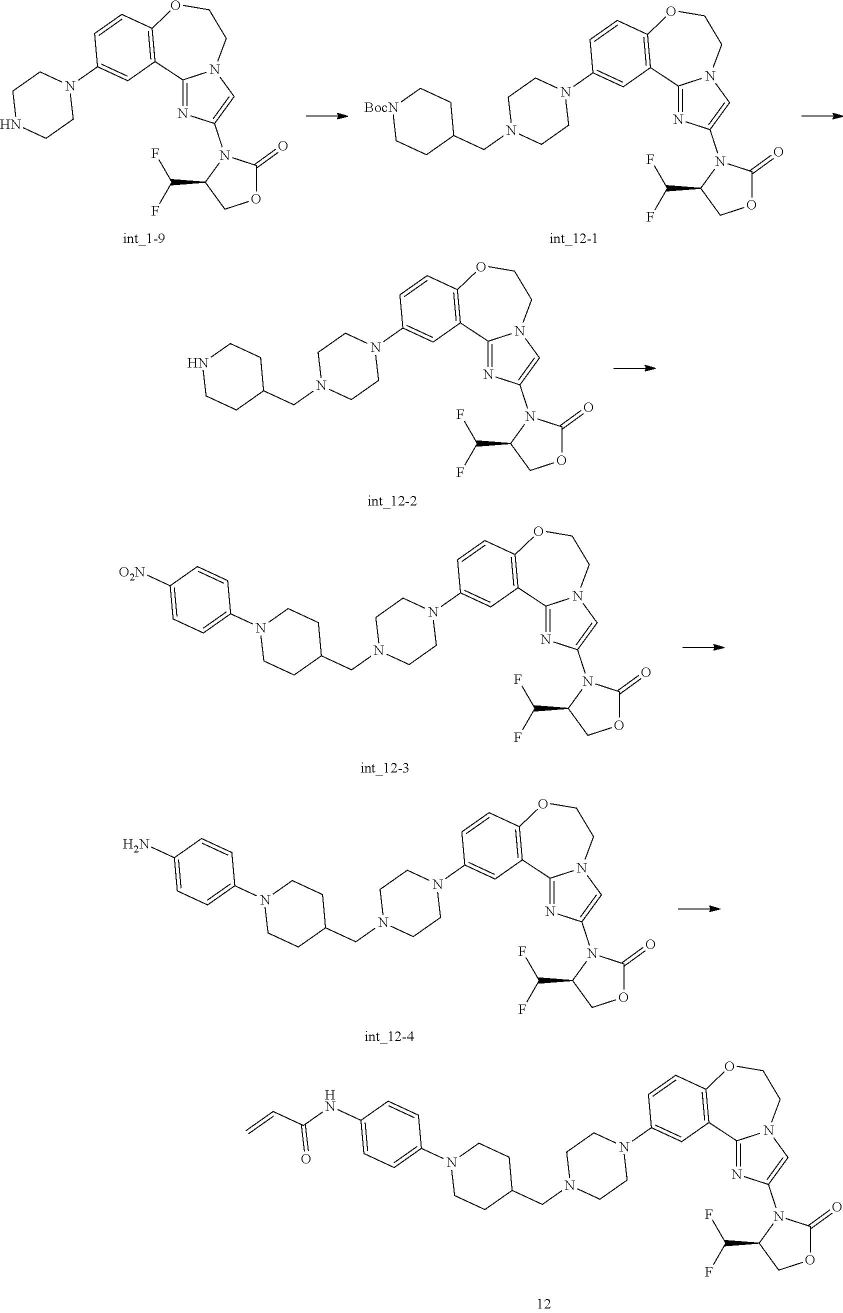 [SMILES: O=C1OC[C@@H](C(F)F)N1c1cn2c(n1)-c1cc(N3CCNCC3)ccc1OCC2 ; CC(C)(C)OC(=O)N1CCC(CN2CCN(c3ccc4c(c3)-c3nc(N5C(=O)OC[C@H]5C(F)F)cn3CCO4)CC2)CC1 ; O=C1OC[C@@H](C(F)F)N1c1cn2c(n1)-c1cc(N3CCN(CC4CCNCC4)CC3)ccc1OCC2 ; O=C1OC[C@@H](C(F)F)N1c1cn2c(n1)-c1cc(N3CCN(CC4CCN(c5ccc([N+](=O)[O-])cc5)CC4)CC3)ccc1OCC2 ; Nc1ccc(N2CCC(CN3CCN(c4ccc5c(c4)-c4nc(N6C(=O)OC[C@H]6C(F)F)cn4CCO5)CC3)CC2)cc1 ; C=CC(=O)Nc1ccc(N2CCC(CN3CCN(c4ccc5c(c4)-c4nc(N6C(=O)OC[C@H]6C(F)F)cn4CCO5)CC3)CC2)cc1]

Step 1: Synthesis of Compound int_12-1:

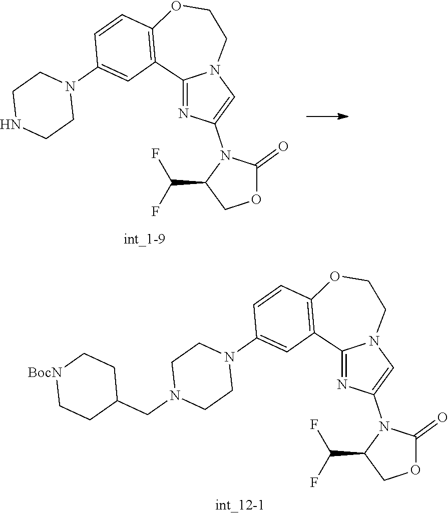 [SMILES: O=C1OC[C@@H](C(F)F)N1c1cn2c(n1)-c1cc(N3CCNCC3)ccc1OCC2 ; CC(C)(C)OC(=O)N1CCC(CN2CCN(c3ccc4c(c3)-c3nc(N5C(=O)OC[C@H]5C(F)F)cn3CCO4)CC2)CC1]

Compound int_1-9 (230 mg, 567.34 μmol) and 1-BOC-4-piperidinecarboxaldehyde (363.00 mg, 1.70 mmol) were dissolved in tetrahydrofuran (8 mL), followed by addition of sodium triacetoxyborohydride (1.20 g, 5.67 mmol) and triethylamine (790 μL, 5.67 mmol). The reaction solution was stirred at 30° C. for 1 h. Water (15 mL) was added to the reaction solution, and the mixed solution was extracted with ethyl acetate (15 mL×3). The organic phases were combined, dried over anhydrous magnesium sulfate, and subjected to suction filtration, and the filtrate was concentrated to dryness under reduced pressure. The residue was then separated and purified by silica gel chromatography to give intermediate int_12-1.

LCMS m/z (ESI): 603.2 [M+H]⁺.

Step 2: Synthesis of Compound int_12-2:

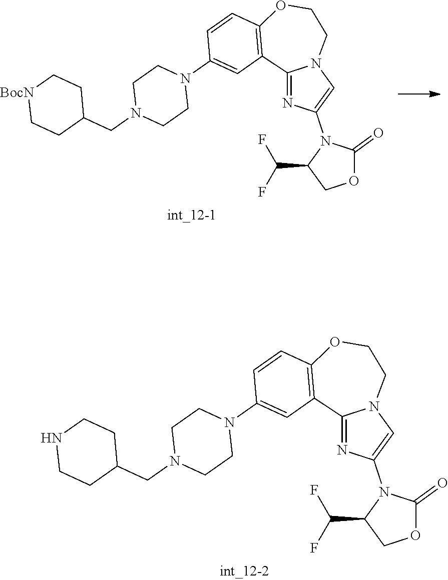 [SMILES: CC(C)(C)OC(=O)N1CCC(CN2CCN(c3ccc4c(c3)-c3nc(N5C(=O)OC[C@H]5C(F)F)cn3CCO4)CC2)CC1 ; O=C1OC[C@@H](C(F)F)N1c1cn2c(n1)-c1cc(N3CCN(CC4CCNCC4)CC3)ccc1OCC2]

Compound int_12-1 (200 mg, 331.86 μmol) was dissolved in dichloromethane (6 mL), followed by addition of trifluoroacetic acid (737 μL, 9.96 mmol). The reaction solution was stirred at 30° C. for 2 h. The reaction solution was concentrated to dryness under reduced pressure to give crude compound int_12-2 as a yellow oil. LCMS m/z (ESI): 503.2 [M+H]⁺.

Step 3: Synthesis of Compound int_12-3:

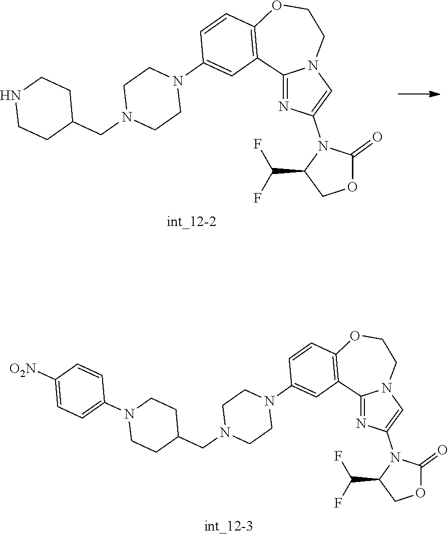 [SMILES: O=C1OC[C@@H](C(F)F)N1c1cn2c(n1)-c1cc(N3CCN(CC4CCNCC4)CC3)ccc1OCC2 ; O=C1OC[C@@H](C(F)F)N1c1cn2c(n1)-c1cc(N3CCN(CC4CCN(c5ccc([N+](=O)[O-])cc5)CC4)CC3)ccc1OCC2]

Compound int_12-2 (140.38 mg, 994.91 μmol) and 4-fluoronitrobenzene (100 mg, 198.98 μmol) were dissolved in N,N-dimethylformamide (3 mL), followed by addition of potassium carbonate (1.00 g, 7.24 mmol). The reaction solution was stirred at 100° C. for 1 h. Water (30 mL) was added to the reaction solution, and the mixed solution was extracted with ethyl acetate (30 mL×3). The organic phases were combined, dried over anhydrous magnesium sulfate, and subjected to suction filtration, and the filtrate was concentrated to dryness under reduced pressure. The residue was then separated and purified by silica gel chromatography to give intermediate int_12-3.

LCMS m/z (ESI): 624.2 [M+H]⁺.

Step 4: Synthesis of Compound int_12-4:

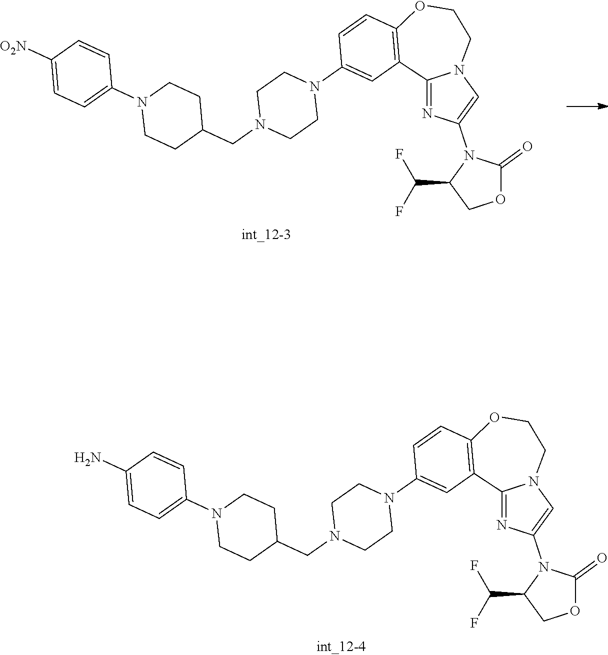 [SMILES: O=C1OC[C@@H](C(F)F)N1c1cn2c(n1)-c1cc(N3CCN(CC4CCN(c5ccc([N+](=O)[O-])cc5)CC4)CC3)ccc1OCC2 ; Nc1ccc(N2CCC(CN3CCN(c4ccc5c(c4)-c4nc(N6C(=O)OC[C@H]6C(F)F)cn4CCO5)CC3)CC2)cc1]

Compound int_12-3 (100 mg, 160.35 μmol) was dissolved in ethanol (5 mL) and water (1 mL), followed by addition of iron powder (89.55 mg, 1.60 mmol) and ammonium chloride (85.77 mg, 1.60 mmol). The reaction solution was stirred at 60° C. for 1 h. After cooling, the reaction solution was filtered, and the filtrate was concentrated to dryness under reduced pressure. A saturated aqueous sodium carbonate solution (15 mL) was added to the reaction solution, and the mixed solution was extracted with dichloromethane (15 mL×3). The organic phases were combined, dried over anhydrous magnesium sulfate, and subjected to suction filtration, and the filtrate was concentrated to dryness under reduced pressure to give crude compound int_12-4 as a yellow solid.

LCMS m/z (ESI): 594.2 [M+H]⁺.

Step 5: Synthesis of Compound int_12-5:

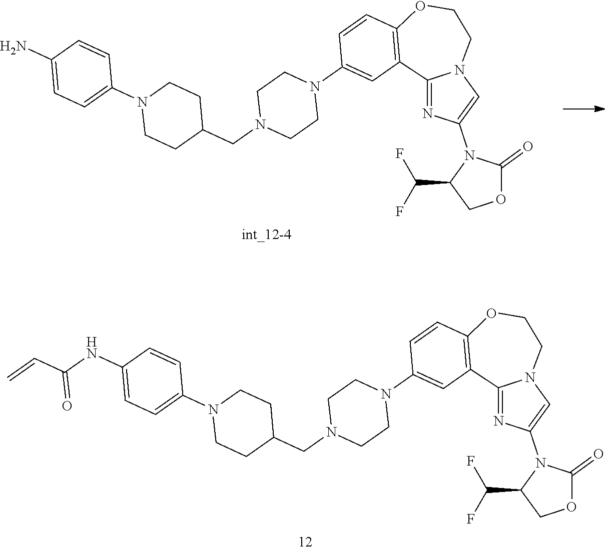 [SMILES: Nc1ccc(N2CCC(CN3CCN(c4ccc5c(c4)-c4nc(N6C(=O)OC[C@H]6C(F)F)cn4CCO5)CC3)CC2)cc1 ; C=CC(=O)Nc1ccc(N2CCC(CN3CCN(c4ccc5c(c4)-c4nc(N6C(=O)OC[C@H]6C(F)F)cn4CCO5)CC3)CC2)cc1]

Compound int_12-4 (50 mg, 84.22 μmol) was dissolved in dichloromethane (3 mL), followed by addition of N,N-diisopropylethylamine (74 μL, 421 μmol). Then, acryloyl chloride (7 μL, 84.22 μmol) was added at 0° C., and the reaction solution was stirred at 30° C. for 5 min. A saturated aqueous sodium carbonate solution (15 mL) was added to the reaction solution, and the mixed solution was extracted with dichloromethane (15 mL×3). The organic phases were combined, dried over anhydrous magnesium sulfate, and subjected to suction filtration, and the filtrate was concentrated to dryness under reduced pressure. The residue was purified by preparative liquid chromatography to give compound 12 as a white solid.

LCMS m/z (ESI): 648.2 [M+H]⁺.

¹H NMR (400 MHz, DMSO-d₆) δ 9.92 (s, 1H), 8.37 (s, 0.349H), 7.77 (br s, 1H), 7.50 (br d, J=9.0 Hz, 2H), 7.34 (s, 1H), 7.06-6.84 (m, 4H), 6.83-6.50 (m, 1H), 6.48-6.39 (m, 1H), 6.25-6.14 (m, 1H), 5.68 (br d, J=9.8 Hz, 1H), 5.12-4.95 (m, 1H), 4.68-4.52 (m, 2H), 4.49-4.32 (m, 4H), 3.62 (br d, J=11.0 Hz, 2H), 3.09 (br s, 4H), 2.61-2.52 (m, 5H), 2.60-2.30 (m, 3H), 1.85-1.75 (m, 2H), 1.70-1.60 (m, 1H), 1.25-1.20 (m, 2H).

### Example 10. Synthesis of Compound 15

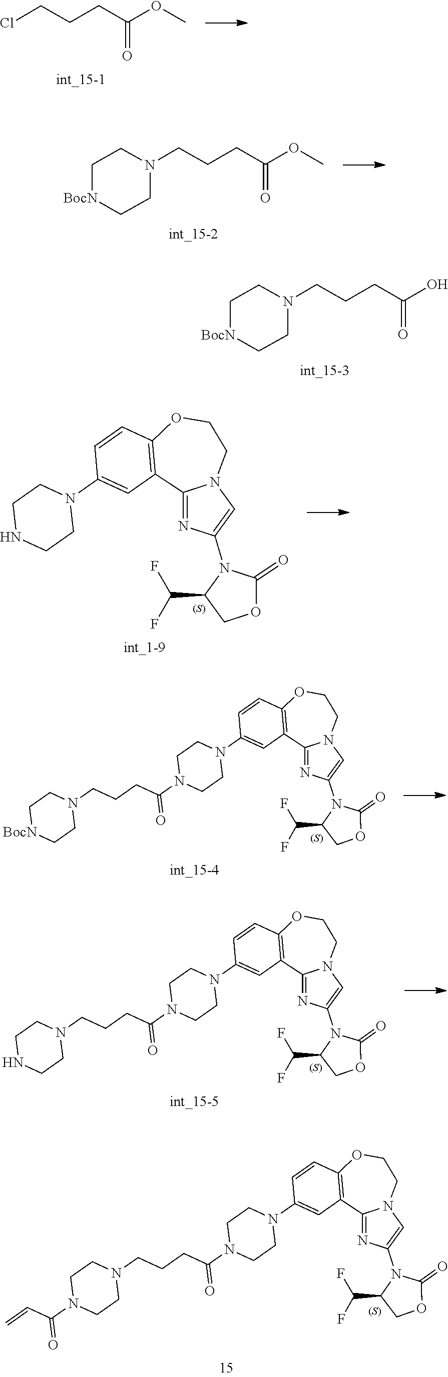

Step 1: Synthesis of Compound int_15-2:

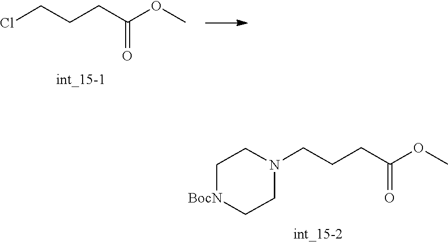

N-Boc-piperazine (10 g, 53.69 mmol) and N,N-diisopropylethylamine (13.85 g, 107.38 mmol) were dissolved in tetrahydrofuran (200 mL), followed by addition of methyl 4-chlorobutyrate (8.8 g, 64.43 mmol). The reaction solution was warmed to 60° C. and stirred for 12 h. After cooling to room temperature, the reaction solution was poured into ice water (100 mL), and the mixed solution was extracted with ethyl acetate (150 mL×3). The organic phases were combined, dried over anhydrous sodium sulfate, and subjected to suction filtration, and the filtrate was concentrated to dryness under reduced pressure. The residue was separated and purified by silica gel chromatography to give intermediate int_15-2.

¹H NMR (400 MHz, CDCl₃) δ 3.65 (s, 3H), 3.42-3.35 (m, 4H), 2.34 (td, J=7.2, 3.1 Hz, 8H), 1.79 (p, J=7.3 Hz, 2H), 1.68 (s, 2H), 1.44 (s, 9H).

Step 2: Synthesis of Compound int_15-3:

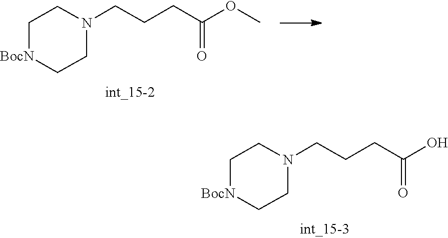 [SMILES: COC(=O)CCCN1CCN(C(=O)OC(C)(C)C)CC1 ; CC(C)(C)OC(=O)N1CCN(CCCC(=O)O)CC1]

Compound int_15-2 (1.7 g, 5.94 mmol) was dissolved in tetrahydrofuran (20 mL), followed by addition of an aqueous solution (10 mL) of lithium hydroxide (1.42 g, 59.36 mmol). The reaction solution was stirred at room temperature for 1 h. The reaction solution was concentrated to remove tetrahydrofuran from the system, and water (20 mL) was added. The mixture was extracted with dichloromethane (20 mL×2), and the aqueous phase was retained. The pH of the aqueous phase was adjusted to 5 to 6 with hydrochloric acid (0.1 N), and the system was extracted with ethyl acetate (50 mL×3). The organic phases were combined, dried over anhydrous sodium sulfate, and subjected to suction filtration, and the filtrate was concentrated to dryness under reduced pressure to give compound int_15-3 as a yellow solid.

Step 3: Synthesis of Compound int_15-4:

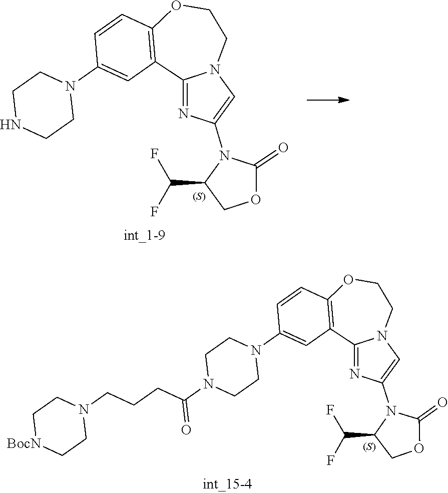

Compound int_1-9 (150 mg, 370 μmol) and compound int_15-3 (151.15 mg, 555 μmol) were dissolved in N,N-dimethylformamide (50 mL), followed by addition of O-(7-azabenzotriazol-1-yl)-N,N,N′-tetramethyluronium hexafluorophosphate (168.72 mg, 444 μmol) and N,N-diisopropylethylamine (143.19 mg, 1.11 mmol). The reaction solution was stirred at room temperature for 2 h. The reaction solution was poured into ice water (50 mL), and the mixed solution was extracted with ethyl acetate (50 mL×3). The organic phases were combined, dried over anhydrous sodium sulfate, and subjected to suction filtration, and the filtrate was concentrated to dryness under reduced pressure. The residue was then separated and purified by silica gel chromatography to give intermediate int_15-4.

LCMS m/z (ESI): 660.4 [M+H]⁺.

Step 4: Synthesis of Compound int_15-5:

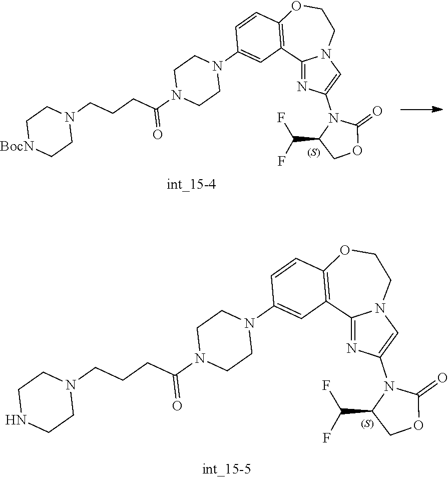

Compound int_15-4 (110 mg, 166.73 μmol) was dissolved in dichloromethane (3 mL), followed by addition of a solution of hydrogen chloride in dioxane (2 mL, 4 N). The reaction solution was stirred at room temperature for 2 h. The reaction solution was concentrated to dryness under reduced pressure to give crude compound int_15-5 as a yellow solid.

### Step 5: Synthesis of Compound 15:

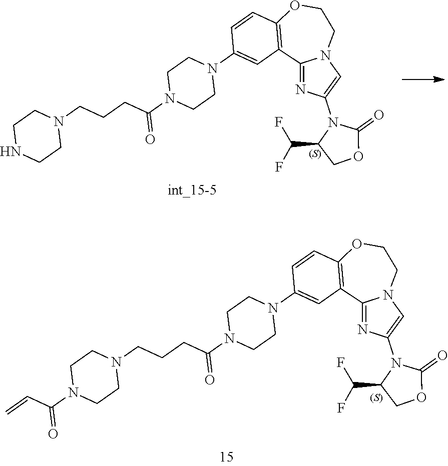

Compound int_15-5 (80 mg) was dissolved in dichloromethane (5 mL), followed by addition of N,N-diisopropylethylamine (36.88 mg, 285.9 μmol). The mixture was cooled to 0° C. in an ice-water bath, and acryloyl chloride (14.23 mg, 157.25 μmol) was added. The ice bath was removed, and the system was stirred for 0.1 h. The reaction solution was poured into a saturated aqueous sodium bicarbonate solution (10 mL), and the mixture was extracted with ethyl acetate (50 mL×3). The organic phases were combined, dried over anhydrous sodium sulfate, and subjected to suction filtration, and the filtrate was concentrated to dryness under reduced pressure. The crude product was purified by preparative liquid chromatography to give compound 15 as a white solid.

LCMS m/z (ESI): 614.3 [M+H]⁺.

¹H NMR (400 MHz, DMSO-d₆) δ 7.82-783 (d, J=4.0 Hz 1H), 7.26 (s, 1H), 6.95-6.97 (br d, J=8.0 Hz, 1H), 6.87-6.90 (dd, J=8.0 Hz, 2H), 6.46-6.61 (m, 2H), 6.26-6.30 (m, 1H), 5.69-5.72 (br d, J=12.0 Hz, 1H), 4.85-4.95 (m, 1H), 4.70-4.74 (m, 1H), 4.51-4.56 (brt, J=16.0 Hz, 1H), 4.39-4.42 (m, 2H), 4.33-4.35 (m, 2H), 3.74-3.80 (m, 4H), 3.63-3.66 (m, 4H), 3.08-3.13 (m, 4H), 2.53-2.60 (m, 5H), 2.42-2.45 (br t, J=12.0 Hz, 2H), 1.87-1.95 (m, 2H).

### Example 11. Synthesis of Compound 16

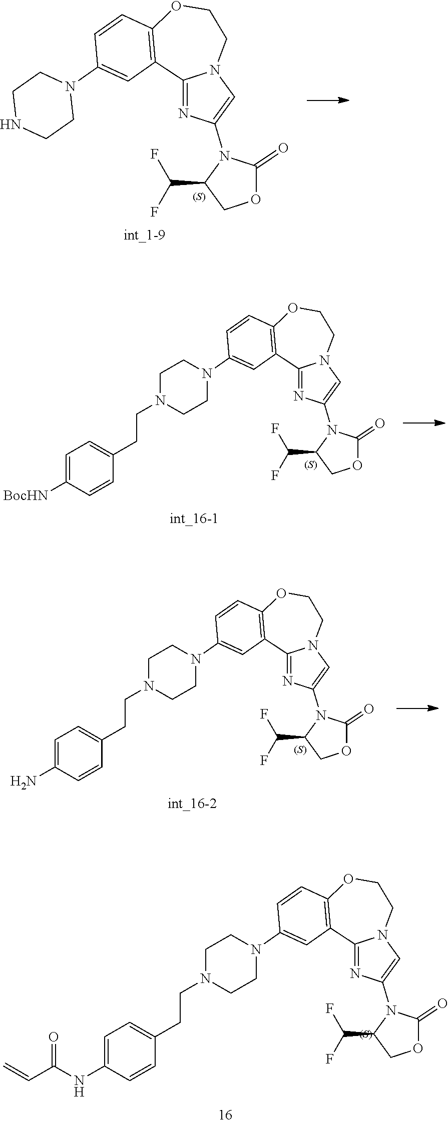

Step 1: Synthesis of Compound int_16-1:

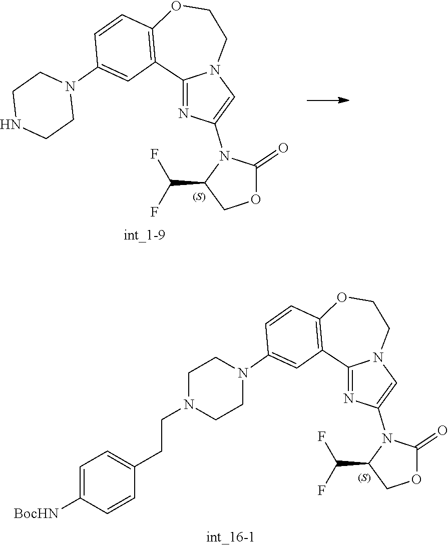

At room temperature, compound int_1-9 (200 mg) and 4-(Boc-amino)phenylacetaldehyde (139.25 mg, 0.592 mmol) were dissolved in a mixed solvent of dichloromethane and methanol (1:1, 5 mL), followed by addition of triethylamine (50.0 mg, 0.494 mmol). The reaction solution was stirred at 25° C. for 0.5 h, and then sodium cyanoborohydride (93.13 mg, 1.482 mmol) was added. The reaction solution was stirred at 25° C. for another 2 h. The reaction solution was poured into ice water (8 mL), and the mixture was extracted with ethyl acetate (30 mL×3). The organic phases were combined, dried over anhydrous sodium sulfate, and subjected to suction filtration, and the filtrate was concentrated to dryness under reduced pressure. The residue was then separated and purified by silica gel chromatography to give intermediate int_16-1.

LCMS m/z (ESI): 625.40 [M+H]⁺.

### Step 2: Synthesis of Compound Int_16-2:

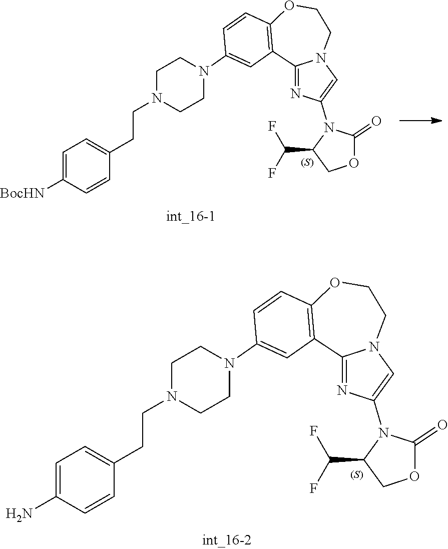

At room temperature, compound int_16-1 (220 mg, 0.55 mmol) was dissolved in dichloromethane (5 mL), followed by addition of a solution of hydrogen chloride in 1,4-dioxane (4 N, 3 mL). The reaction solution was stirred at 25° C. for 2 h. The reaction solution was concentrated to dryness under reduced pressure to give crude compound int_16-2 as a yellow solid.

LCMS m/z (ESI): 525.30 [M+H]⁺.

Step 3: Synthesis of Compound int_16-3:

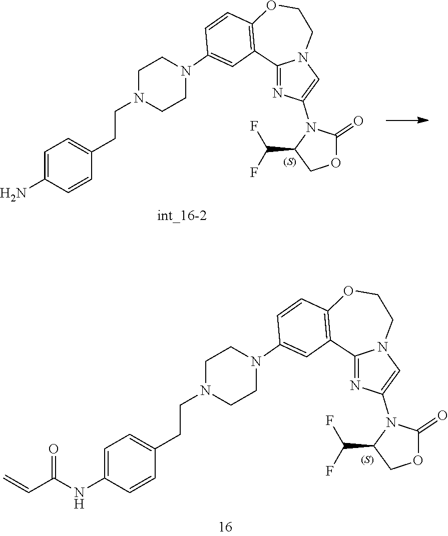

Compound int_16-2 (150 mg) was dissolved in anhydrous dichloromethane (3 mL), followed by addition of N,N-diisopropylethylamine (69.0 mg, 0.534 mmol). At 0° C., acryloyl chloride (24.2 mg, 0.267 mmol) was added. The reaction solution was stirred at 25° C. for 0.1 h. The reaction solution was diluted with a saturated aqueous sodium bicarbonate solution (6 mL), and the mixed solution was extracted with ethyl acetate (10 mL×3). The organic phases were combined, dried over anhydrous sodium sulfate, and subjected to suction filtration, and the filtrate was concentrated to dryness under reduced pressure. The residue was separated and purified by silica gel chromatography to give compound 16.

LCMS m/z (ESI): 579 [M+H]⁺.

¹H NMR (400 MHz, CDCl₃) δ 7.85 (d, J=2.9 Hz, 1H), 7.50 (d, J=8.1 Hz, 2H), 7.25 (m, 1H), 7.19 (d, J=8.3 Hz, 3H), 6.95 (d, J=8.9 Hz, 1H), 6.89 (dd, J=8.9, 2.8 Hz, 1H), 6.51-6.37 (m, 1H), 6.22 (dd, J=16.8, 10.2 Hz, 1H), 5.76 (dd, J=10.2, 1.3 Hz, 1H), 4.93 (d, J=6.5 Hz, 1H), 4.87 (d, J=8.9 Hz, 1H), 4.72 (dd, J=9.3, 4.0 Hz, 1H), 4.52 (t, J=9.3 Hz, 1H), 4.43-4.37 (m, 2H), 4.36-4.30 (m, 2H), 3.22 (t, J=5.0 Hz, 4H), 2.89-2.81 (m, 2H), 2.80-2.73 (m, 4H), 2.73-2.65 (m, 2H).

### Example 12. Synthesis of Compound 27

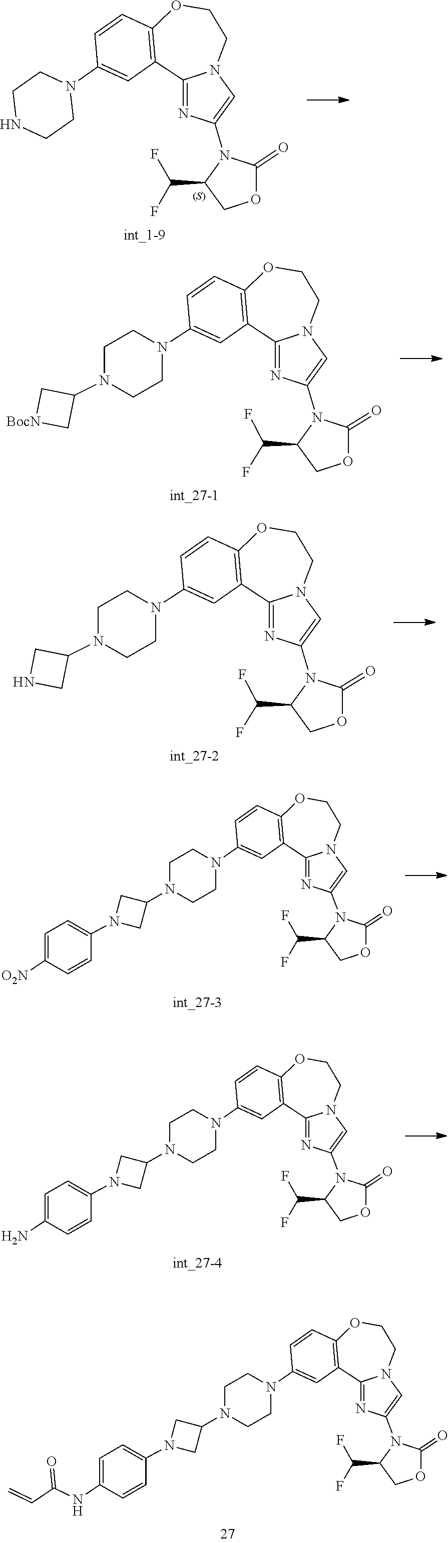

Step 1: Synthesis of Compound int_27-1:

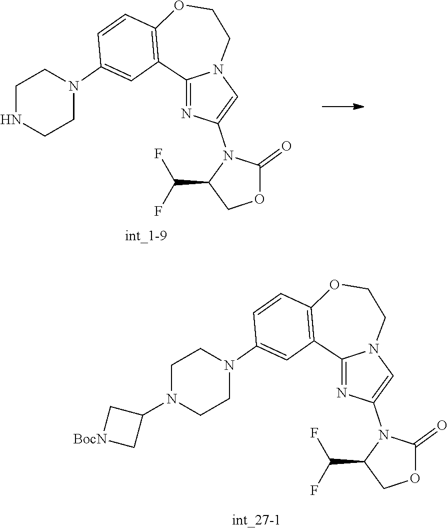 [SMILES: O=C1OC[C@@H](C(F)F)N1c1cn2c(n1)-c1cc(N3CCNCC3)ccc1OCC2 ; CC(C)(C)OC(=O)N1CC(N2CCN(c3ccc4c(c3)-c3nc(N5C(=O)OC[C@H]5C(F)F)cn3CCO4)CC2)C1]

Compound int_1-9 (600 mg) and 1-Boc-3-azetidinone (930 mg, 5.43 mmol) were dissolved in dichloromethane (25 mL), followed by addition of sodium triacetoxyborohydride (1.15 g, 5.43 mmol). The reaction solution was stirred at 25° C. for 16 h. The reaction solution was quenched with a saturated aqueous ammonium chloride solution (200 mL) and extracted with dichloromethane (30 mL×3). The organic phases were combined, dried over anhydrous sodium sulfate, and subjected to suction filtration, and the filtrate was concentrated to dryness under reduced pressure. The residue was then separated and purified by silica gel chromatography to give compound int_27-1.

LCMS m/z (ESI): 561.3 [M+H]⁺.

Step 2: Synthesis of Compound int_27-2:

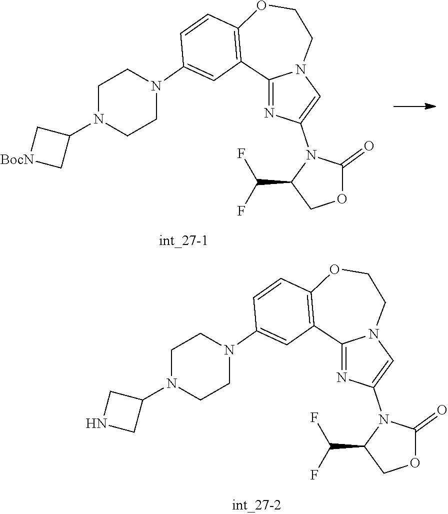 [SMILES: CC(C)(C)OC(=O)N1CC(N2CCN(c3ccc4c(c3)-c3nc(N5C(=O)OC[C@H]5C(F)F)cn3CCO4)CC2)C1 ; O=C1OC[C@@H](C(F)F)N1c1cn2c(n1)-c1cc(N3CCN(C4CNC4)CC3)ccc1OCC2]

Compound int_27-1 (559 mg, 1.00 mmol) was dissolved in dichloromethane (10 mL), followed by addition of trifluoroacetic acid (10 mL). The reaction solution was stirred at 25° C. for 2 h. A saturated aqueous sodium bicarbonate solution was added to the reaction solution to adjust the pH to 9, and the mixed solution was extracted with dichloromethane (50 mL×3). The organic phases were combined, dried over anhydrous sodium sulfate, and subjected to suction filtration, and the filtrate was concentrated to dryness under reduced pressure to give crude yellow oil int_27-2.

LCMS m/z (ESI): 461.0 [M+H]⁺.

Step 3: Synthesis of Compound int_27-3:

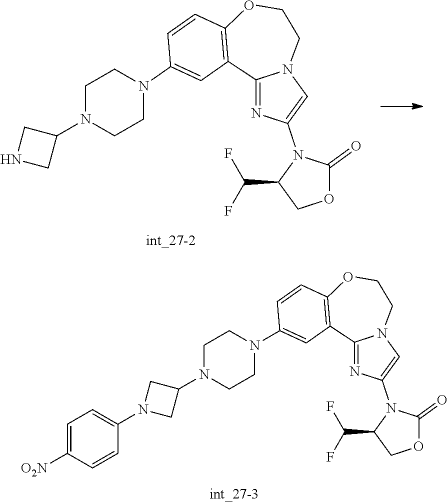 [SMILES: O=C1OC[C@@H](C(F)F)N1c1cn2c(n1)-c1cc(N3CCN(C4CNC4)CC3)ccc1OCC2 ; O=C1OC[C@@H](C(F)F)N1c1cn2c(n1)-c1cc(N3CCN(C4CN(c5ccc([N+](=O)[O-])cc5)C4)CC3)ccc1OCC2]

Compound int_27-2 (310 mg), 4-fluoronitrobenzene (190 mg, 1.35 mmol), and anhydrous potassium carbonate (279 mg, 2.02 mmol) were dissolved in N,N-dimethylformamide (25 mL). The reaction solution was stirred at 100° C. for 1 h. After cooling, the reaction solution was quenched with ice water (200 mL) and extracted with ethyl acetate (30 mL×3). The organic phases were combined, washed with saturated brine (30 mL×3), dried over anhydrous sodium sulfate, and subjected to suction filtration, and the filtrate was concentrated to dryness under reduced pressure. The residue was separated and purified by silica gel chromatography to give compound int_27-3.

LCMS m/z (ESI): 582.1 [M+H]⁺.

Step 4: Synthesis of Compound int_27-4:

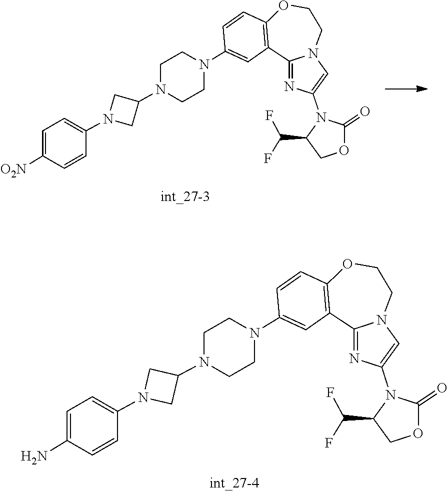 [SMILES: O=C1OC[C@@H](C(F)F)N1c1cn2c(n1)-c1cc(N3CCN(C4CN(c5ccc([N+](=O)[O-])cc5)C4)CC3)ccc1OCC2 ; Nc1ccc(N2CC(N3CCN(c4ccc5c(c4)-c4nc(N6C(=O)OC[C@H]6C(F)F)cn4CCO5)CC3)C2)cc1]

Compound int_27-3 (386 mg, 0.66 mmol), reduced iron powder (394 mg, 6.64 mmol), and ammonium chloride (355 mg, 6.64 mmol) were dissolved in a mixture of ethanol and water (6:1, 100 mL). The reaction solution was stirred at 60° C. for 2 h. The reaction solution was hot-filtered through diatomite and rinsed with ethanol, and then the filtrate was concentrated to dryness under reduced pressure. A saturated aqueous sodium bicarbonate solution was added until the pH reached 9, and the mixed solution was extracted with ethyl acetate (50 mL×3). The organic phases were combined, dried over anhydrous sodium sulfate, and subjected to suction filtration, and the filtrate was concentrated to dryness under reduced pressure to give compound int_27-4 as a yellow solid.

LCMS m/z (ESI): 551.8 [M+H]⁺.

¹H NMR (400 MHz, DMSO-d₆) δ 7.73 (d, J=2.9 Hz, 1H), 7.30 (s, 1H), 6.96-6.84 (m, 2H), 6.88-6.50 (m, 1H), 6.47-6.40 (m, 2H), 6.23-6.17 (m, 2H), 5.02-4.94 (m, 1H), 4.62-4.49 (m, 2H), 4.39-4.29 (m, 4H), 3.78 (t, J=6.8 Hz, 2H), 3.39 (t, J=6.3 Hz, 2H), 3.20 (m, 5H), 3.06 (m, 4H).

Step 5: Synthesis of Compound int_27:

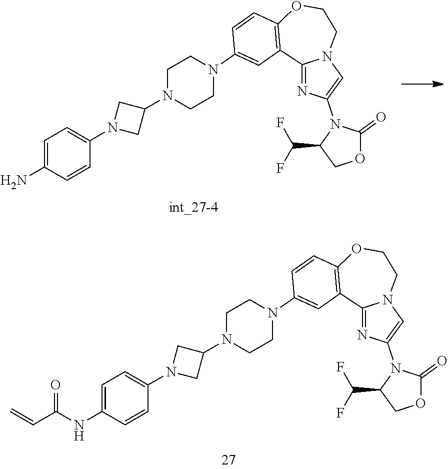 [SMILES: Nc1ccc(N2CC(N3CCN(c4ccc5c(c4)-c4nc(N6C(=O)OC[C@H]6C(F)F)cn4CCO5)CC3)C2)cc1 ; C=CC(=O)Nc1ccc(N2CC(N3CCN(c4ccc5c(c4)-c4nc(N6C(=O)OC[C@H]6C(F)F)cn4CCO5)CC3)C2)cc1]

Compound int_27-4 (360 mg, 0.65 mmol) was dissolved in dichloromethane (20 mL), followed by addition of N,N-diisopropylethylamine (169 mg, 1.31 mmol). The reaction solution was cooled to 0° C., and then acryloyl chloride (59 mg, 0.65 mmol) was slowly added. The reaction solution was stirred at 0° C. for 15 min. The reaction solution was quenched with a saturated aqueous sodium bicarbonate solution (50 mL) and extracted with dichloromethane (50 mL×3). The organic phases were combined, dried over anhydrous sodium sulfate, and subjected to suction filtration, and the filtrate was concentrated to dryness under reduced pressure. The residue was purified by slurrying with ethyl acetate (20 mL) to give white compound 27.

LCMS m/z (ESI): 605.8 [M+H]⁺.

¹H NMR (400 MHz, DMSO-d₆) δ 9.84 (s, 1H), 7.73 (d, J=2.9 Hz, 1H), 7.47-7.41 (m, 2H), 7.31 (s, 1H), 6.96-6.84 (m, 2H), 6.78-6.50 (m, 1H), 6.41-6.29 (m, 3H), 6.15 (dd, J=17.0, 2.2 Hz, 1H), 5.64 (dd, J=10.1, 2.2 Hz, 1H), 4.96 (m, 1H), 4.65-4.47 (m, 2H), 4.40-4.29 (m, 4H), 3.88 (m, 2H), 3.59-3.52 (m, 2H), 3.25 (m, 5H), 3.06 (m, 4H).

### Example 13. Synthesis of Compound 28 and Compound 29

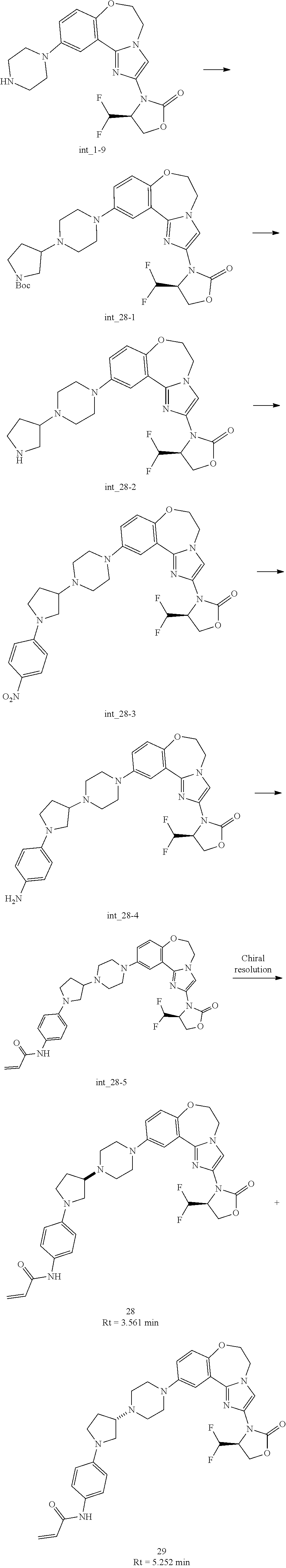

Step 1: Synthesis of Compound int_28-1:

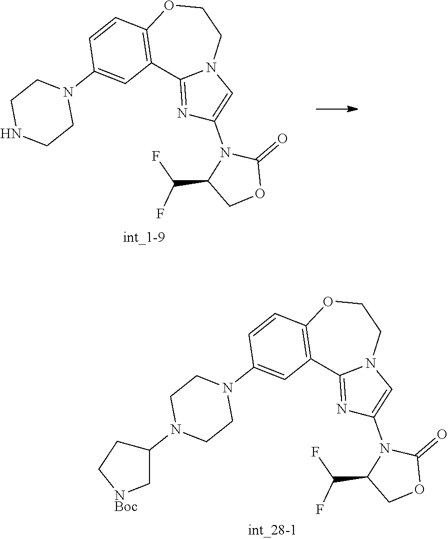 [SMILES: O=C1OC[C@@H](C(F)F)N1c1cn2c(n1)-c1cc(N3CCNCC3)ccc1OCC2 ; CC(C)(C)OC(=O)N1CCC(N2CCN(c3ccc4c(c3)-c3nc(N5C(=O)OC[C@H]5C(F)F)cn3CCO4)CC2)C1]

Compound int_1-9 (3.30 g) and 1-tert-butoxycarbonyl-3-pyrrolidinone (2.07 g, 11.2 mmol) were dissolved in anhydrous methanol (100 mL), followed by addition of triethylamine (776 mg, 7.47 mmol) and glacial acetic acid (45 mg, 0.75 mmol). The reaction solution was stirred at 40° C. for 1 h, and then sodium cyanoborohydride (1.41 g, 22.41 mmol) was added. The reaction solution was stirred at 40° C. for 2 h. The reaction solution was quenched with a saturated aqueous ammonium chloride solution (200 mL) and extracted with dichloromethane (50 mL×3). The organic phases were combined, dried over anhydrous sodium sulfate, and subjected to suction filtration, and the filtrate was concentrated to dryness under reduced pressure. The residue was then separated and purified by silica gel chromatography to give compound int_28-1.

LCMS m/z (ESI): 575.3 [M+H]⁺.

Step 2: Synthesis of Compound int_28-2:

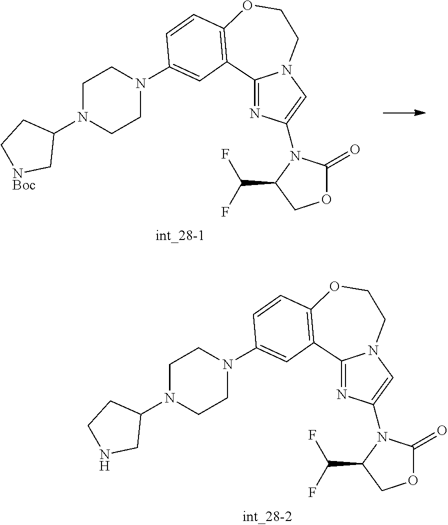 [SMILES: CC(C)(C)OC(=O)N1CCC(N2CCN(c3ccc4c(c3)-c3nc(N5C(=O)OC[C@H]5C(F)F)cn3CCO4)CC2)C1 ; O=C1OC[C@@H](C(F)F)N1c1cn2c(n1)-c1cc(N3CCN(C4CCNC4)CC3)ccc1OCC2]

Compound int_28-1 (3.9 g, 6.79 mmol) was dissolved in dichloromethane (200 mL), followed by addition of trifluoroacetic acid (10 mL). The reaction solution was stirred at 25° C. for 1 h. A saturated aqueous sodium bicarbonate solution was added to the reaction solution until the pH reached 9, and the mixed solution was extracted with dichloromethane (50 mL×3). The organic phases were combined, dried over anhydrous sodium sulfate, and subjected to suction filtration, and the filtrate was concentrated to dryness under reduced pressure to give crude compound int_28-2 as a yellow oil.

LCMS m/z (ESI): 475.2 [M+H]⁺.

Step 3: Synthesis of Compound int_28-3:

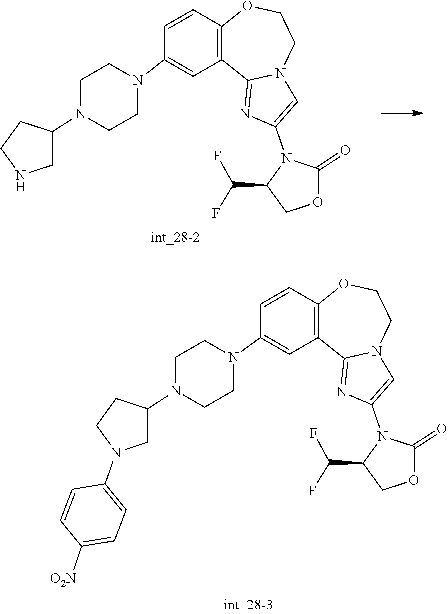 [SMILES: O=C1OC[C@@H](C(F)F)N1c1cn2c(n1)-c1cc(N3CCN(C4CCNC4)CC3)ccc1OCC2 ; O=C1OC[C@@H](C(F)F)N1c1cn2c(n1)-c1cc(N3CCN(C4CCN(c5ccc([N+](=O)[O-])cc5)C4)CC3)ccc1OCC2]

Compound int_28-2 (3.2 g), 4-fluoronitrobenzene (1.90 g, 13.49 mmol), and anhydrous potassium carbonate (2.80 g, 20.23 mmol) were dissolved in N,N-dimethylformamide (250 mL). The reaction solution was stirred at 100° C. for 1 h. After the reaction solution was cooled, water (2 L) was added, and the mixture was extracted with ethyl acetate (200 mL×3). The organic phases were combined, washed with saturated brine (200 mL×3), dried over anhydrous sodium sulfate, and subjected to suction filtration, and the filtrate was concentrated to dryness under reduced pressure. The residue was separated and purified by silica gel chromatography to give compound int_28-3.

LCMS m/z (ESI): 596.2 [M+H]⁺.

Step 4: Synthesis of Compound int_28-4:

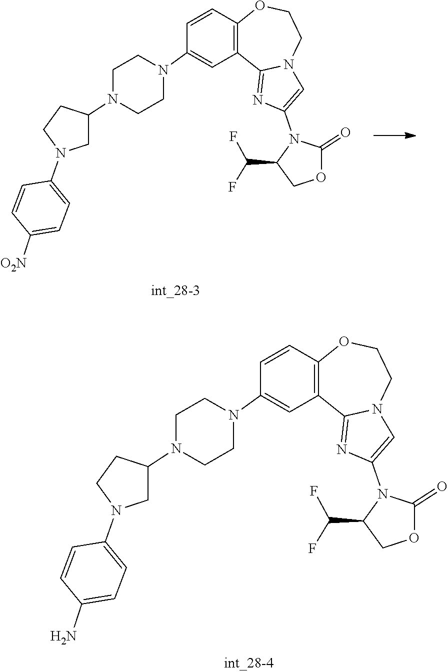 [SMILES: O=C1OC[C@@H](C(F)F)N1c1cn2c(n1)-c1cc(N3CCN(C4CCN(c5ccc([N+](=O)[O-])cc5)C4)CC3)ccc1OCC2 ; Nc1ccc(N2CCC(N3CCN(c4ccc5c(c4)-c4nc(N6C(=O)OC[C@H]6C(F)F)cn4CCO5)CC3)C2)cc1]

Compound int_28-3 (2 g, 3.36 mmol), reduced iron powder (1.88 g, 33.58 mmol), and ammonium chloride (1.80 g, 33.58 mmol) were dissolved in a mixture of ethanol and water (6:1, 500 mL). The reaction solution was stirred at 60° C. for 2 h. The reaction solution was hot-filtered through diatomite and rinsed with ethanol, and then the filtrate was concentrated to dryness under reduced pressure. A saturated aqueous sodium bicarbonate solution was added until the pH reached 9, and the mixed solution was extracted with ethyl acetate (100 mL×3). The organic phases were combined, dried over anhydrous sodium sulfate, and subjected to suction filtration, and the filtrate was concentrated to dryness under reduced pressure to give compound int_28-4 as a yellow solid.

LCMS m/z (ESI): 566.20 [M+H]⁺.

### Step 5: Synthesis of Compound Int_28-5:

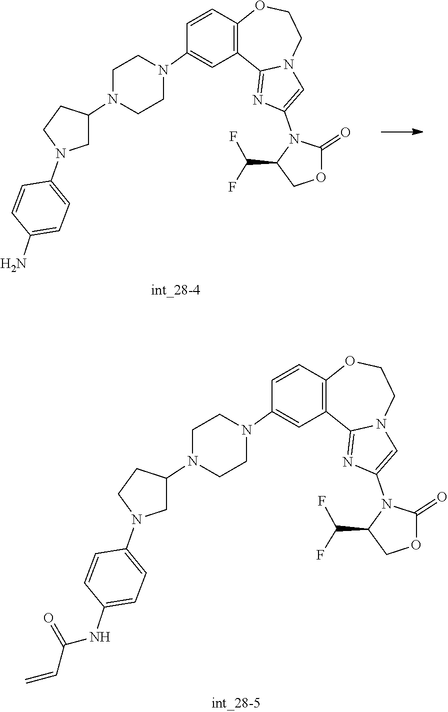 [SMILES: Nc1ccc(N2CCC(N3CCN(c4ccc5c(c4)-c4nc(N6C(=O)OC[C@H]6C(F)F)cn4CCO5)CC3)C2)cc1 ; C=CC(=O)Nc1ccc(N2CCC(N3CCN(c4ccc5c(c4)-c4nc(N6C(=O)OC[C@H]6C(F)F)cn4CCO5)CC3)C2)cc1]

Compound int_28-4 (1.69 g, 2.75 mmol) was dissolved in dichloromethane (50 mL), followed by addition of N,N-diisopropylethylamine (709.71 mg, 5.49 mmol). The reaction solution was cooled to 0° C., and acryloyl chloride (254 mg, 2.75 mmol) was slowly added. The reaction solution was stirred at 0° C. for 0.5 h. The reaction solution was quenched with a saturated aqueous sodium bicarbonate solution (50 mL) and extracted with dichloromethane (50 mL×3). The organic phases were combined, dried over anhydrous sodium sulfate, and subjected to suction filtration, and the filtrate was concentrated to dryness under reduced pressure. The residue was purified by slurrying with ethyl acetate (20 mL) to give white compound int_28-5.

LCMS m/z (ESI): 620.3 [M+H]⁺.

¹H NMR (400 MHz, DMSO-d₆) δ 9.78 (s, 1H), 7.75 (d, J=2.8 Hz, 1H), 7.48-7.41 (m, 2H), 7.31 (s, 1H), 6.96-6.85 (m, 2H), 6.79-6.52 (m, 1H), 6.50-6.46 (m, 2H), 6.35 (dd, J=17.0, 10.1 Hz, 1H), 6.14 (dd, J=17.0, 2.2 Hz, 1H), 5.63 (dd, J=10.1, 2.2 Hz, 1H), 5.00 (m, 1H), 4.60 (d, J=9.3 Hz, 1H), 4.56-4.50 (m, 1H), 4.40-4.29 (m, 4H), 3.44 (dd, J=9.0, 7.1 Hz, 1H), 3.36-3.26 (m, 1H), 3.18 (m, 1H), 3.06 (m, 5H), 2.94 (m, 1H), 2.61 (m, 4H), 2.17 (dd, J=11.4, 5.7 Hz, 1H), 1.82 (m, 1H).

### Step 6: Synthesis of Compound 28 and Compound 29:

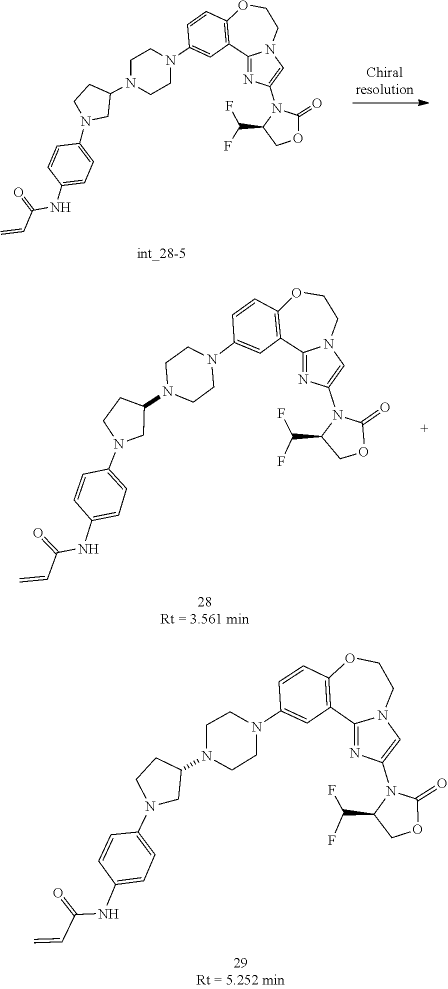 [SMILES: C=CC(=O)Nc1ccc(N2CCC(N3CCN(c4ccc5c(c4)-c4nc(N6C(=O)OC[C@H]6C(F)F)cn4CCO5)CC3)C2)cc1 ; C=CC(=O)Nc1ccc(N2CC[C@@H](N3CCN(c4ccc5c(c4)-c4nc(N6C(=O)OC[C@H]6C(F)F)cn4CCO5)CC3)C2)cc1 ; C=CC(=O)Nc1ccc(N2CC[C@H](N3CCN(c4ccc5c(c4)-c4nc(N6C(=O)OC[C@H]6C(F)F)cn4CCO5)CC3)C2)cc1]

Compound int_28-5 was subjected to chiral resolution to give compound 28 (Rt=3.561 min) and compound 29 (Rt=5.252 min).

### Chiral Analysis Method:

Instrument: Waters UPCC with PDA Detector and QDa Detector
Column: Chiralcel OJ-3, 100×4.6 mm I.D., 3 μm
Mobile phase: A: carbon dioxide; B: methanol (0.05% diethylamine)
Elution gradient: 40% B
Flow rate: 2.8 mL/min
Column temperature: 35° C.
Back pressure: 1500 psi
Detection wavelength: 254 nm

### Example 14. Synthesis of Compound 99

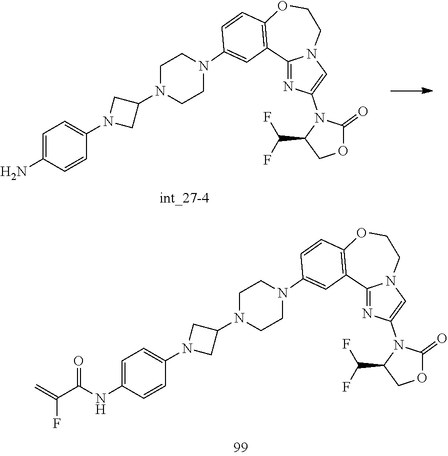 [SMILES: Nc1ccc(N2CC(N3CCN(c4ccc5c(c4)-c4nc(N6C(=O)OC[C@H]6C(F)F)cn4CCO5)CC3)C2)cc1 ; C=C(F)C(=O)Nc1ccc(N2CC(N3CCN(c4ccc5c(c4)-c4nc(N6C(=O)OC[C@H]6C(F)F)cn4CCO5)CC3)C2)cc1]

Compound int_27-4 (130 mg, 0.236 mmol) and 2-fluoroacrylic acid (32 mg, 0.353 mmol) were dissolved in N,N-dimethylacetamide (5 mL), followed by addition of N,N,N′,N′-tetramethyl-O-(7-azabenzotriazol-1-yl)uronium hexafluorophosphate (134 mg, 0.353 mmol) and N,N-diisopropylethylamine (91 mg, 0.708 mmol). The reaction solution was stirred at 25° C. for 2 h. Water (5 mL) was added to the reaction solution, and the mixture was extracted with ethyl acetate (20 mL×3). The organic phases were combined, sequentially washed with saturated brine (20 mL×3), dried over anhydrous sodium sulfate, and subjected to suction filtration, and the filtrate was concentrated to dryness under reduced pressure. The residue was then slurried with N,N-dimethylformamide to give compound 99 as a yellow solid.

LCMS m/z (ESI): 624.0 [M+H]⁺.

¹H NMR (400 MHz, DMSO-d₆) δ 10.07-9.97 (m, 1H), 7.77 (d, J=2.8 Hz, 1H), 7.54-7.46 (m, 2H), 7.34 (s, 1H), 6.98-6.89 (m, 2H), 6.85-6.51 (m, 1H), 6.47-6.38 (m, 2H), 5.77-5.54 (m, 1H), 5.34 (dd, J=15.7, 3.5 Hz, 1H), 5.09-4.94 (m, 1H), 4.66-4.53 (m, 2H), 4.43-4.35 (m, 4H), 3.93 (t, J=7.1 Hz, 2H), 3.61 (dd, J=7.5, 5.4 Hz, 2H), 3.32 (m, 5H), 3.11 (s, 4H).

### Example 15. Synthesis of Compound 100

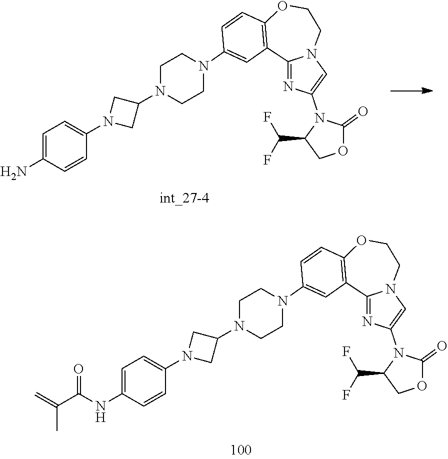 [SMILES: Nc1ccc(N2CC(N3CCN(c4ccc5c(c4)-c4nc(N6C(=O)OC[C@H]6C(F)F)cn4CCO5)CC3)C2)cc1 ; C=C(C)C(=O)Nc1ccc(N2CC(N3CCN(c4ccc5c(c4)-c4nc(N6C(=O)OC[C@H]6C(F)F)cn4CCO5)CC3)C2)cc1]

Compound int_27-4 (130 mg, 0.235 mmol) was dissolved in dichloromethane (5 mL), followed by addition of N,N-diisopropylethylamine (91 mg, 0.707 mmol). The mixture was cooled to 0° C. in an ice-water bath, and methacryloyl chloride (25 mg, 0.235 mmol) was added. The reaction solution was stirred for 1 h. The reaction solution was poured into a saturated aqueous sodium bicarbonate solution (25 mL), and the mixture was extracted with dichloromethane (20 mL×3). The organic phases were combined, dried over anhydrous sodium sulfate, and subjected to suction filtration, and the filtrate was concentrated to dryness under reduced pressure. The residue was then purified by preparative liquid chromatography to give compound 100 as a white solid.

LCMS m/z (ESI): 620.0 [M+H]⁺.

¹H NMR (400 MHz, DMSO-d₆) δ 9.48 (s, 1H), 7.77 (d, J=2.8 Hz, 1H), 7.45 (d, J=8.3 Hz, 2H), 7.34 (s, 1H), 6.98-6.89 (m, 2H), 6.82-6.52 (m, 1H), 6.40 (d, J=8.3 Hz, 2H), 5.74 (s, 1H), 5.43 (s, 1H), 5.07-4.96 (m, 1H), 4.64-4.54 (m, 2H), 4.40 (d, J=5.6 Hz, 2H), 4.37 (d, J=5.2 Hz, 2H), 3.92 (t, J=7.0 Hz, 2H), 3.59 (t, J=6.4 Hz, 2H), 3.30 (t, J=6.2 Hz, 5H), 3.13-3.08 (m, 4H), 1.93 (s, 3H).

### Example 16. Synthesis of Compound 106

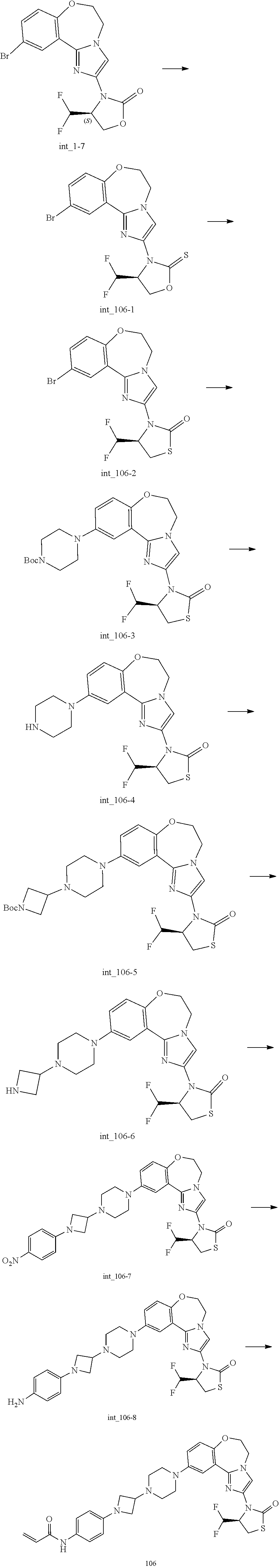

Step 1: Synthesis of Compound int_106-1:

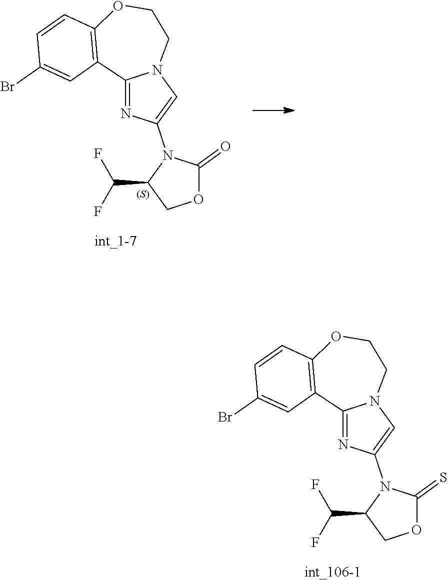

Intermediate int_1-7 (2.00 g, 5.00 mmol) was dissolved in anhydrous methylbenzene (70 mL), followed by addition of Lawesson's reagent (20.21 g, 49.98 mmol). The reaction solution was stirred at 130° C. for 12 h. The reaction solution was concentrated to give a pale yellow crude product, which was separated and purified by silica gel chromatography to give compound int_106-1.

Step 2: Synthesis of Compound int_106-2:

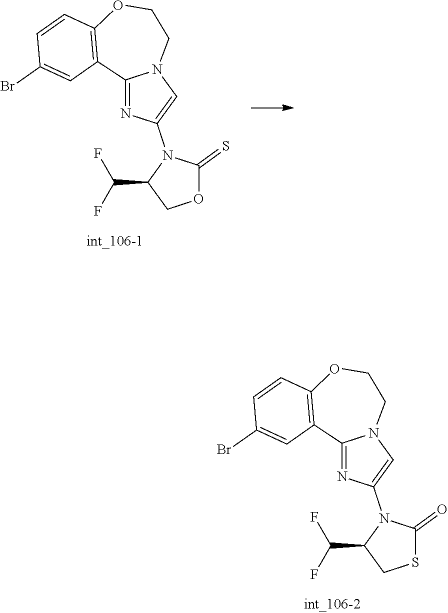 [SMILES: FC(F)[C@@H]1COC(=S)N1c1cn2c(n1)-c1cc(Br)ccc1OCC2 ; O=C1SC[C@@H](C(F)F)N1c1cn2c(n1)-c1cc(Br)ccc1OCC2]

Compound int_106-1 (150 mg, 0.360 mmol) was dissolved in anhydrous methylbenzene (8 mL), followed by addition of 2-dicyclohexylphosphino-2′,6′-dimethoxybiphenyl (45 mg, 0.109 mmol) and dichloro(p-cymene)ruthenium(II) dimer (67 mg, 0.109 mmol). The reaction solution was stirred at 110° C. for 12 h. The reaction solution was concentrated to dryness to give a black crude product, which was separated and purified by silica gel chromatography to give compound int_106-2.

Step 3: Synthesis of Compound int_106-3:

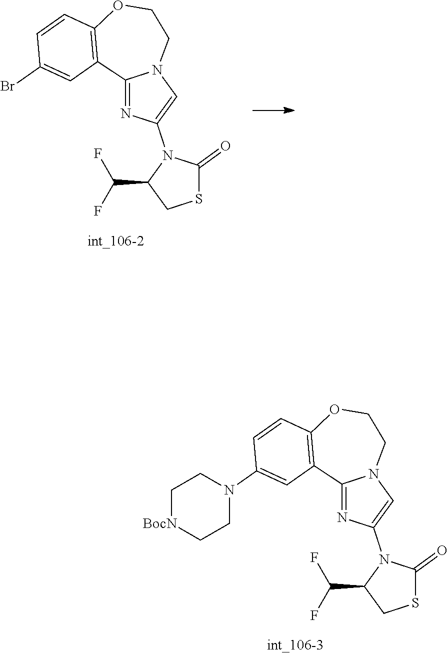 [SMILES: O=C1SC[C@@H](C(F)F)N1c1cn2c(n1)-c1cc(Br)ccc1OCC2 ; CC(C)(C)OC(=O)N1CCN(c2ccc3c(c2)-c2nc(N4C(=O)SC[C@H]4C(F)F)cn2CCO3)CC1]

Compound int_106-2 (450 mg, 1.08 mmol) was dissolved in 1,4-dioxane (10 mL), followed by addition of tert-butyl piperazine-1-carboxylate (403 mg, 2.16 mmol), cesium carbonate (704 mg, 2.16 mmol), and (2-dicyclohexylphosphino-2′,6′-diisopropoxy-1,1′-biphenyl)[2-(2′-amino-1,1′-biphenyl)]palladium(II) methanesulfonate (136 mg, 0.324 mmol). The reaction solution was stirred at 105° C. for 16 h. The reaction solution was concentrated to dryness to give a brown crude product, which was separated and purified by silica gel chromatography to give compound int_106-3.

LCMS m/z (ESI): 522.2 [M+H]⁺.

Step 4: Synthesis of Compound int_106-4:

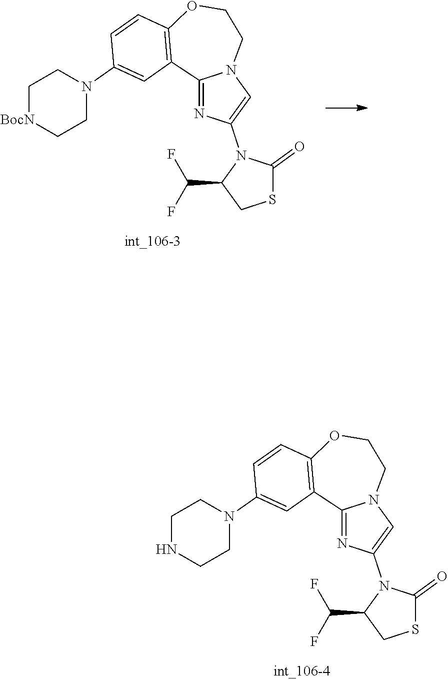 [SMILES: CC(C)(C)OC(=O)N1CCN(c2ccc3c(c2)-c2nc(N4C(=O)SC[C@H]4C(F)F)cn2CCO3)CC1 ; O=C1SC[C@@H](C(F)F)N1c1cn2c(n1)-c1cc(N3CCNCC3)ccc1OCC2]

Compound int_106-3 (375 mg, 0.72 mmol) was dissolved in dichloromethane (5 mL), followed by addition of a solution of hydrogen chloride in dioxane (4 M, 5 mL). The reaction solution was stirred at 25° C. for 1 h. The reaction solution was concentrated to dryness under reduced pressure to give crude compound int_106-4 as a white solid.

LCMS m/z (ESI): 421.90 [M+H]⁺.

Step 5: Synthesis of Compound int_106-5:

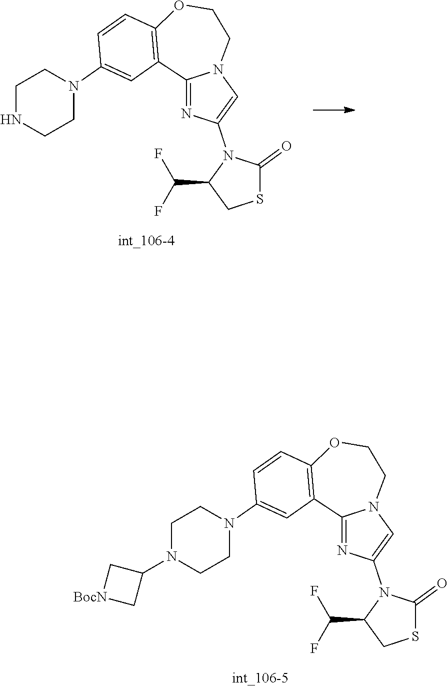 [SMILES: O=C1SC[C@@H](C(F)F)N1c1cn2c(n1)-c1cc(N3CCNCC3)ccc1OCC2 ; CC(C)(C)OC(=O)N1CC(N2CCN(c3ccc4c(c3)-c3nc(N5C(=O)SC[C@H]5C(F)F)cn3CCO4)CC2)C1]

Compound int_106-4 (312 mg) and 1-Boc-3-azetidinone (507 mg, 2.96 mmol) were dissolved in dichloromethane (25 mL), followed by addition of sodium triacetoxyborohydride (628 mg, 2.96 mmol). The reaction solution was stirred at 25° C. for 2 h. The reaction solution was quenched with water (25 mL) and extracted with dichloromethane (25 mL×3). The organic phases were combined, dried over anhydrous sodium sulfate, and filtered, and the filtrate was concentrated to dryness under reduced pressure. The residue was separated and purified by silica gel chromatography to give compound int_106-5.

LCMS m/z (ESI): 576.90[M+H]⁺.

Step 6: Synthesis of Compound int_106-6:

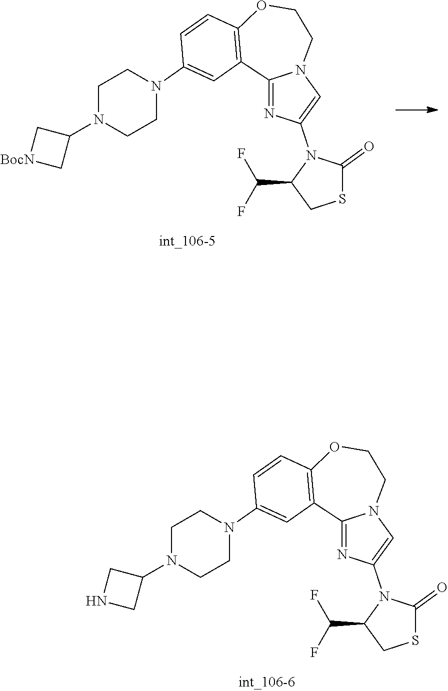 [SMILES: CC(C)(C)OC(=O)N1CC(N2CCN(c3ccc4c(c3)-c3nc(N5C(=O)SC[C@H]5C(F)F)cn3CCO4)CC2)C1 ; O=C1SC[C@@H](C(F)F)N1c1cn2c(n1)-c1cc(N3CCN(C4CNC4)CC3)ccc1OCC2]

Compound int_106-5 (290 mg, 0.506 mmol) was dissolved in dichloromethane (10 mL), followed by dropwise addition of trifluoroacetic acid (3 mL). The reaction solution was stirred at 25° C. for 1 h. The reaction solution was concentrated to dryness under reduced pressure to give crude compound int_106-6 as a white solid.

LCMS m/z (ESI): 476.90[M+H]⁺.

Step 7: Synthes of Compound int_106-7:

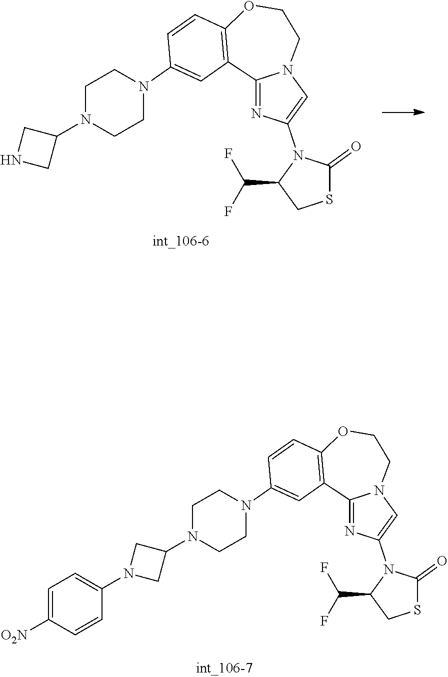 [SMILES: O=C1SC[C@@H](C(F)F)N1c1cn2c(n1)-c1cc(N3CCN(C4CNC4)CC3)ccc1OCC2 ; O=C1SC[C@@H](C(F)F)N1c1cn2c(n1)-c1cc(N3CCN(C4CN(c5ccc([N+](=O)[O-])cc5)C4)CC3)ccc1OCC2]

Compound int_106-6 (220 mg), 4-fluoronitrobenzene (130 mg, 0.824 mmol), and potassium phosphate (197 mg, 1.428 mmol) were dissolved in N—N-dimethylformamide (25 mL). The reaction solution was heated to 100° C. and stirred for 2 h. After the reaction solution was cooled to room temperature, water (25 mL) was added, and the mixture was extracted with ethyl acetate (25 mL×3). The organic phases were combined, dried over anhydrous sodium sulfate, and subjected to suction filtration, and the filtrate was concentrated to dryness under reduced pressure. The residue was separated and purified by silica gel chromatography to give compound int_106-7.

LCMS m/z (ESI): 597.90 [M+H]⁺.

Step 8: Synthesis of Compound int_106-8:

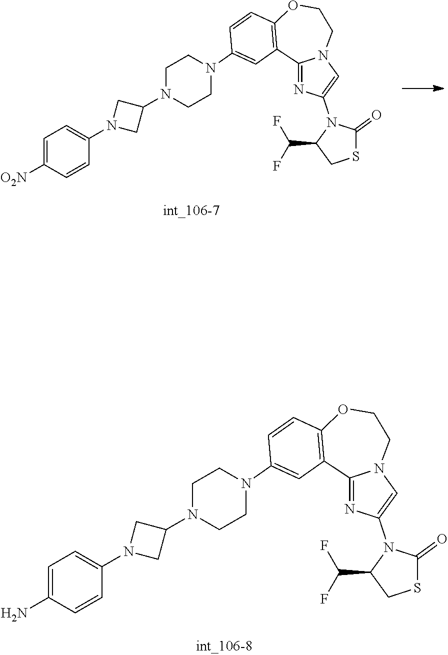 [SMILES: O=C1SC[C@@H](C(F)F)N1c1cn2c(n1)-c1cc(N3CCN(C4CN(c5ccc([N+](=O)[O-])cc5)C4)CC3)ccc1OCC2 ; Nc1ccc(N2CC(N3CCN(c4ccc5c(c4)-c4nc(N6C(=O)SC[C@H]6C(F)F)cn4CCO5)CC3)C2)cc1]

Compound int_106-7 (240 mg, 0.402 mmol), reduced iron powder (224 mg, 4.02 mmol), and ammonium chloride (215 mg, 4.02 mmol) were mixed in water (2 mL) and ethanol (12 mL). The reaction solution was heated to 80° C. and stirred for 1 h. The reaction solution was cooled to room temperature and then filtered, and the filtrate was concentrated to dryness under reduced pressure to give crude compound int_106-8 as a white solid.

LCMS m/z (ESI): 567.9[M+H]⁺.

### Ste 9: Synthesis of Compound 106:

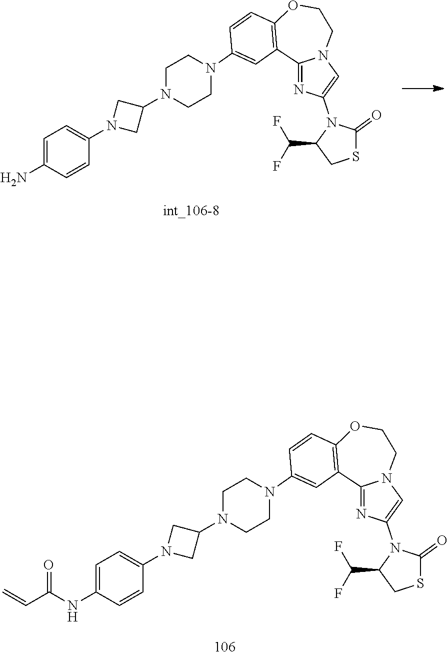 [SMILES: Nc1ccc(N2CC(N3CCN(c4ccc5c(c4)-c4nc(N6C(=O)SC[C@H]6C(F)F)cn4CCO5)CC3)C2)cc1 ; C=CC(=O)Nc1ccc(N2CC(N3CCN(c4ccc5c(c4)-c4nc(N6C(=O)SC[C@H]6C(F)F)cn4CCO5)CC3)C2)cc1]

Compound int_106-8 (180 mg) was dissolved in dichloromethane (10 mL), followed by addition of N,N-diisopropylethylamine (0.5 mL). At 0° C., acryloyl chloride (29 mg, 0.322 mmol) was added dropwise with stirring. The reaction solution was stirred at 25° C. for 1 h. Water (25 mL) was added, and the mixture was extracted with dichloromethane (10 mL×3). The organic phases were combined, dried over anhydrous sodium sulfate, and subjected to suction filtration, and the filtrate was concentrated to dryness under reduced pressure. The residue was separated and purified by silica gel chromatography to give compound 106.

LCMS m/z (ESI): 622.0 [M+H]⁺.

¹H NMR (400 MHz, DMSO-d₆) δ 9.86 (d, J=2.5 Hz, 1H), 7.77 (t, J=2.8 Hz, 1H), 7.51-7.44 (m, 3H), 6.99-6.89 (m, 2H), 6.73-6.34 (m, 4H), 6.22-6.14 (m, 1H), 5.77-5.64 (m, 1H), 5.26 (d, J=22.2 Hz, 1H), 4.42-4.33 (m, 4H), 3.95-3.84 (m, 3H), 3.62-3.50 (m, 3H), 3.31-3.28 (m, 1H), 3.10 (s, 4H), 2.72-2.52 (m, 4H).

### Example 17. Synthesis of Compound 95

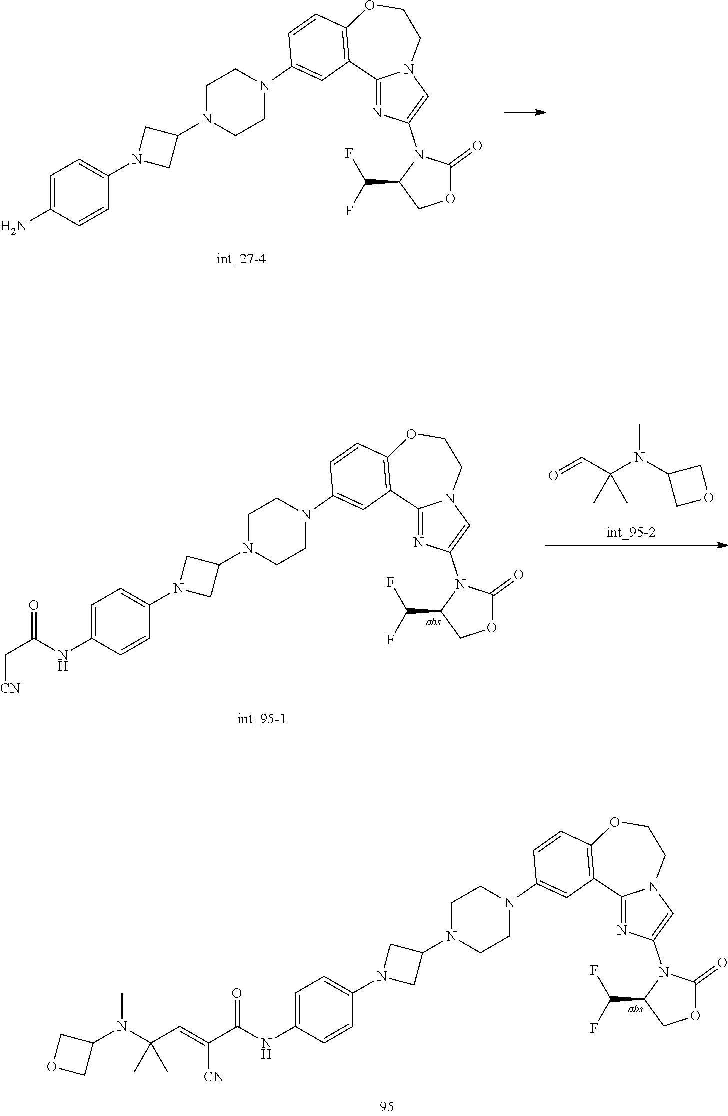 [SMILES: Nc1ccc(N2CC(N3CCN(c4ccc5c(c4)-c4nc(N6C(=O)OC[C@H]6C(F)F)cn4CCO5)CC3)C2)cc1 ; N#CCC(=O)Nc1ccc(N2CC(N3CCN(c4ccc5c(c4)-c4nc(N6C(=O)OC[C@H]6C(F)F)cn4CCO5)CC3)C2)cc1 ; CN(C1COC1)C(C)(C)C=O ; CN(C1COC1)C(C)(C)/C=C(\C#N)C(=O)Nc1ccc(N2CC(N3CCN(c4ccc5c(c4)-c4nc(N6C(=O)OC[C@H]6C(F)F)cn4CCO5)CC3)C2)cc1]

Step 1: Synthesis of Compound int_95-1:

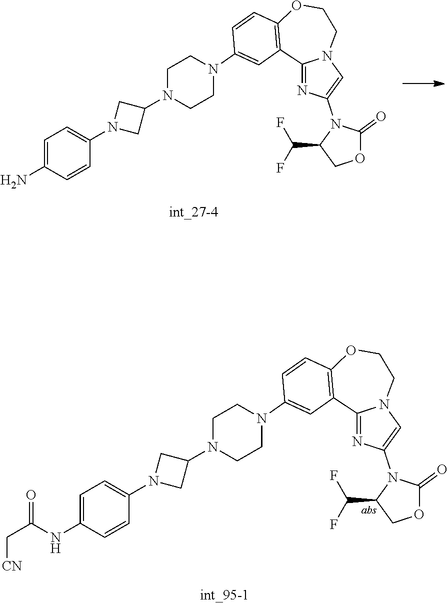 [SMILES: Nc1ccc(N2CC(N3CCN(c4ccc5c(c4)-c4nc(N6C(=O)OC[C@H]6C(F)F)cn4CCO5)CC3)C2)cc1 ; N#CCC(=O)Nc1ccc(N2CC(N3CCN(c4ccc5c(c4)-c4nc(N6C(=O)OC[C@H]6C(F)F)cn4CCO5)CC3)C2)cc1]

Compound int_27-4 (400 mg), N,N,N′,N′-tetramethyl-O-(7-azabenzotriazol-1-yl)uronium hexafluorophosphate (331 mg, 0.87 mmol), and cyanoacetic acid (68 mg, 0.80 mmol) were dissolved in N,N-dimethylformamide (30 mL), followed by addition of diisopropylethylamine (281 mg, 2.18 mmol). The reaction solution was stirred at 25° C. for 16 h. Water (300 mL) was added to the reaction solution, and the mixture was extracted with ethyl acetate (50 mL×3). The organic phases were combined, washed with saturated brine (50 mL×3), dried over anhydrous sodium sulfate, and subjected to suction filtration, and the filtrate was concentrated to dryness under reduced pressure. The residue was purified by slurrying with ethyl acetate (20 mL) to give compound int_95-1 as a white solid.

LCMS nm/z (ESI): 619.3 [M+H]⁺.

### Step 2: Synthesis of Compound 95:

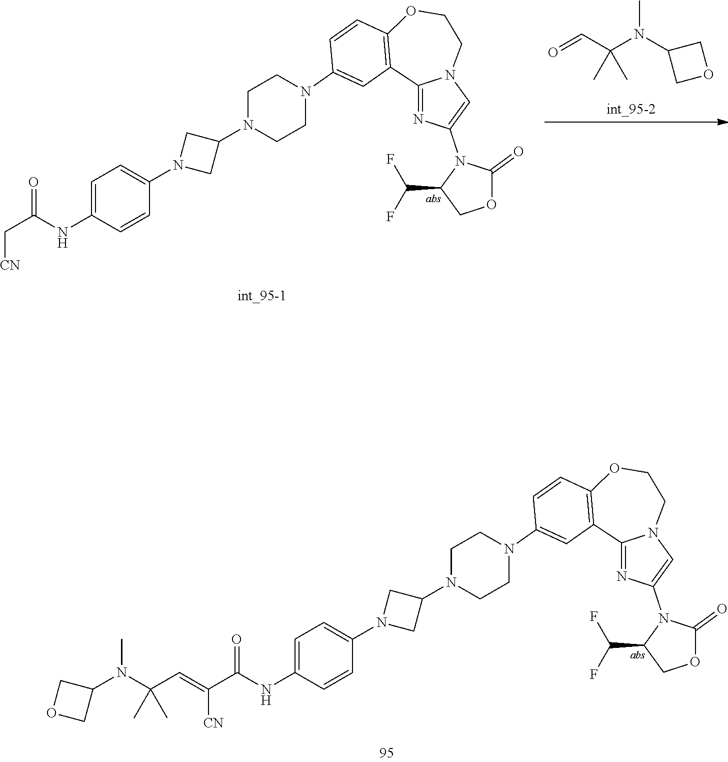 [SMILES: N#CCC(=O)Nc1ccc(N2CC(N3CCN(c4ccc5c(c4)-c4nc(N6C(=O)OC[C@H]6C(F)F)cn4CCO5)CC3)C2)cc1 ; CN(C1COC1)C(C)(C)C=O ; CN(C1COC1)C(C)(C)/C=C(\C#N)C(=O)Nc1ccc(N2CC(N3CCN(c4ccc5c(c4)-c4nc(N6C(=O)OC[C@H]6C(F)F)cn4CCO5)CC3)C2)cc1]

2-Methyl-2-(methyl(oxetan-3-yl)amino)propanal (508 mg, 3.23 mmol) was dissolved in dichloromethane (30 mL). After nitrogen purging, the reaction solution was cooled to 0° C., followed by addition of pyrrolidine (0.4 mL) and trimethylsilyl chloride (0.4 mL). The reaction solution was warmed to 25° C. and stirred for 1 h, and then compound int_95-1 (400 mg, 0.65 mmol) was added. The reaction solution was stirred at 25° C. for another 1 h. The reaction solution was quenched with water (30 mL) and extracted with dichloromethane (30 mL×3). The organic phases were combined, dried over anhydrous sodium sulfate, and subjected to suction filtration, and the filtrate was concentrated to dryness under reduced pressure. The residue was then purified by preparative liquid chromatography to give compound 95 as a white solid.

LCMS m/z (ESI): 758.4 [M+H]⁺.

¹H NMR (400 MHz, DMSO-d₆) δ 9.90 (s, 1H), 7.77 (d, J=2.9 Hz, 1H), 7.40 (d, J=8.6 Hz, 2H), 7.34 (s, 1H), 7.30 (s, 1H), 6.99-6.89 (m, 2H), 6.83-6.52 (m, 1H), 6.43 (d, J=8.8 Hz, 2H), 5.07-4.96 (m, 1H), 4.67-4.59 (m, 3H), 4.56 (dd, J=9.4, 3.8 Hz, 1H), 4.51 (t, J=6.9 Hz, 2H), 4.40-4.37 (m, 4H), 4.19-4.09 (m, 1H), 3.94 (t, J=7.0 Hz, 2H), 3.61 (t, J=6.5 Hz, 2H), 3.29 (m, 1H), 3.11 (s, 4H), 2.53 (m, 4H), 2.33 (s, 3H), 1.18 (s, 6H).

### Example 18. Synthesis of Compound 96

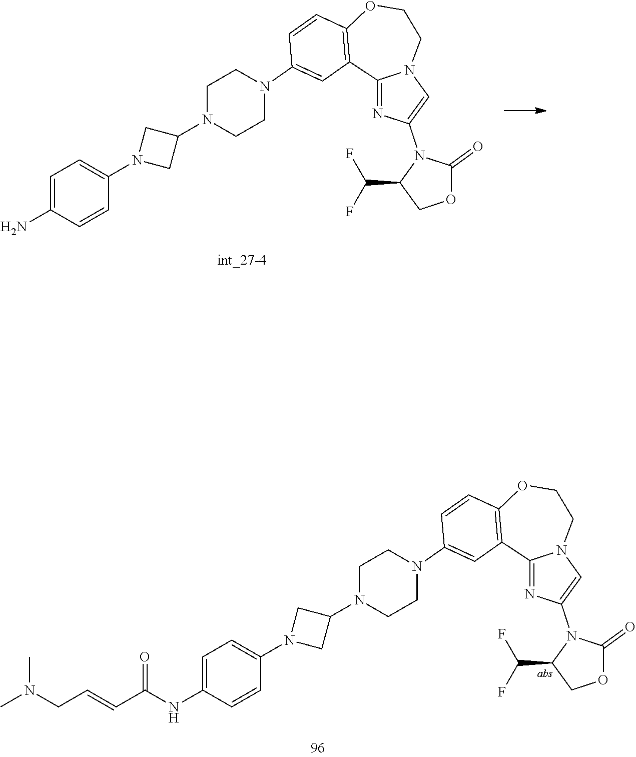 [SMILES: Nc1ccc(N2CC(N3CCN(c4ccc5c(c4)-c4nc(N6C(=O)OC[C@H]6C(F)F)cn4CCO5)CC3)C2)cc1 ; CN(C)C/C=C/C(=O)Nc1ccc(N2CC(N3CCN(c4ccc5c(c4)-c4nc(N6C(=O)OC[C@H]6C(F)F)cn4CCO5)CC3)C2)cc1]

Compound int_27-4 (200 mg), N,N,N′,N′-tetramethyl-O-(7-azabenzotriazol-1-yl)uronium hexafluorophosphate (166 mg, 0.44 mmol), and trans-4-dimethylaminocrotonic acid hydrochloride (66 mg, 0.40 mmol) were dissolved in N,N-dimethylformamide (30 mL), followed by addition of diisopropylethylamine (187.45 mg, 1.45 mmol). The reaction solution was stirred at 25° C. for 16 h. Water (300 mL) was added to the reaction solution, and the mixture was extracted with ethyl acetate (50 mL×3). The organic phases were combined, washed with saturated brine (50 mL×3), dried over anhydrous sodium sulfate, and subjected to suction filtration, and the filtrate was concentrated to dryness under reduced pressure. The residue was then slurried with ethyl acetate to give compound 96 as a white solid.

LCMS m/z (ESI): 663.4 [M+H]+.

¹H NMR (400 MHz, DMSO-d₆) δ 9.76 (s, 1H), 7.79-7.69 (m, 1H), 7.45-7.43 (m, 2H), 7.34-7.28 (m, 1H), 6.96-6.84 (m, 2H), 6.81-6.48 (m, 2H), 6.42-6.33 (m, 2H), 6.19 (m, 1H), 4.98 (m, 1H), 4.63-4.51 (in, 2H), 4.34 (m, 4H), 3.88 (m, 2H), 3.60-3.53 (m, 2H), 3.26 (m, 2H), 3.23-3.02 (m, 5H), 2.52 (m, 4H), 2.21 (s, 6H).

The target compounds 8-344 in Table 1 could be obtained by synthetic methods similar to those in Examples 1-18 described above using different starting materials.

TABLE 1

| | | MS |
|---|---|---|
| Compound | Compound structure | (M + H)⁺ |
| 8 | 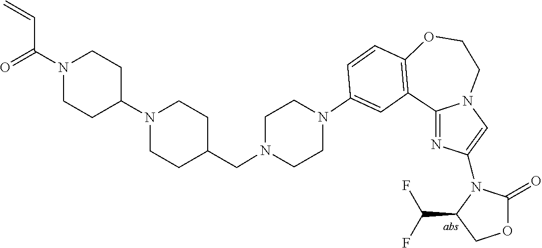 [SMILES: C=CC(=O)N1CCC(N2CCC(CN3CCN(c4ccc5c(c4)-c4nc(N6C(=O)OC[C@H]6C(F)F)cn4CCO5)CC3)CC2)CC1] | 640 |
| 9 | 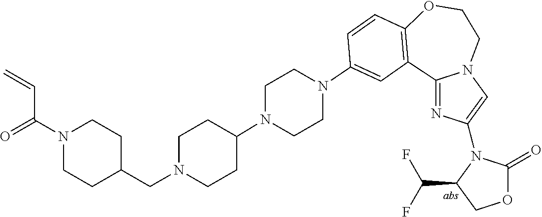 [SMILES: C=CC(=O)N1CCC(CN2CCC(N3CCN(c4ccc5c(c4)-c4nc(N6C(=O)OC[C@H]6C(F)F)cn4CCO5)CC3)CC2)CC1] | 640 |
| 10 | 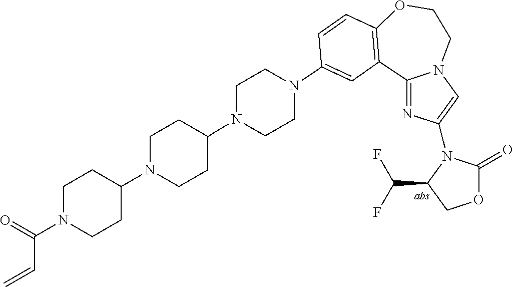 [SMILES: C=CC(=O)N1CCC(N2CCC(N3CCN(c4ccc5c(c4)-c4nc(N6C(=O)OC[C@H]6C(F)F)cn4CCO5)CC3)CC2)CC1] | 626 |
| 13 | 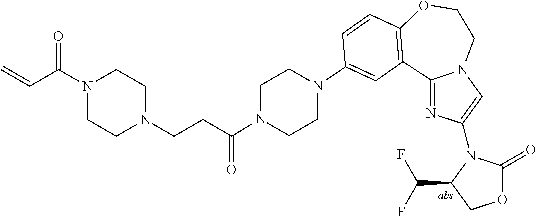 [SMILES: C=CC(=O)N1CCN(CCC(=O)N2CCN(c3ccc4c(c3)-c3nc(N5C(=O)OC[C@H]5C(F)F)cn3CCO4)CC2)CC1] | 600 |
| 14 | 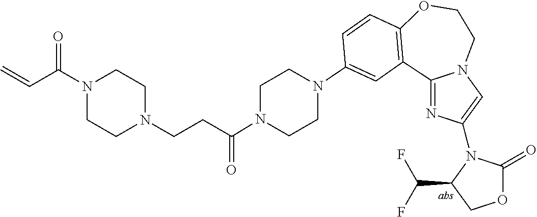 [SMILES: C=CC(=O)N1CCN(CCC(=O)N2CCN(c3ccc4c(c3)-c3nc(N5C(=O)OC[C@H]5C(F)F)cn3CCO4)CC2)CC1] | 597 |
| 17 | 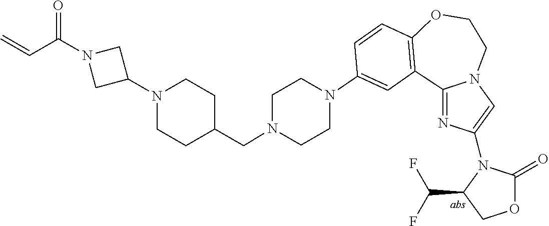 [SMILES: C=CC(=O)N1CC(N2CCC(CN3CCN(c4ccc5c(c4)-c4nc(N6C(=O)OC[C@H]6C(F)F)cn4CCO5)CC3)CC2)C1] | 612 |
| 18 | 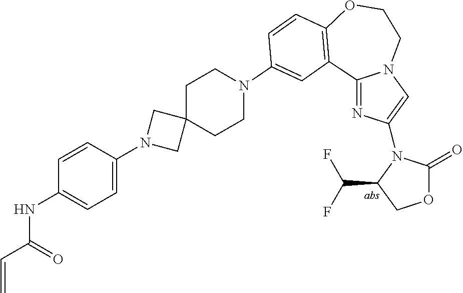 [SMILES: C=CC(=O)Nc1ccc(N2CC3(CCN(c4ccc5c(c4)-c4nc(N6C(=O)OC[C@H]6C(F)F)cn4CCO5)CC3)C2)cc1] | 591 |
| 19 | 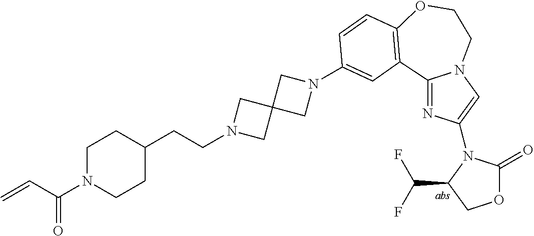 [SMILES: C=CC(=O)N1CCC(CCN2CC3(C2)CN(c2ccc4c(c2)-c2nc(N5C(=O)OC[C@H]5C(F)F)cn2CCO4)C3)CC1] | 583 |
| 20 | 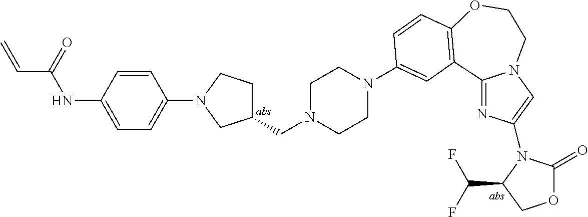 [SMILES: C=CC(=O)Nc1ccc(N2CC[C@H](CN3CCN(c4ccc5c(c4)-c4nc(N6C(=O)OC[C@H]6C(F)F)cn4CCO5)CC3)C2)cc1] | 634 |
| 21 | 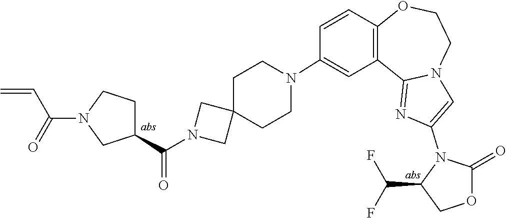 [SMILES: C=CC(=O)N1CC[C@@H](C(=O)N2CC3(CCN(c4ccc5c(c4)-c4nc(N6C(=O)OC[C@H]6C(F)F)cn4CCO5)CC3)C2)C1] | 597 |
| 22 | 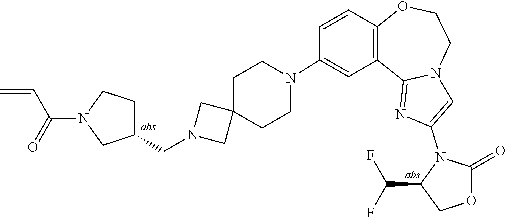 [SMILES: C=CC(=O)N1CC[C@H](CN2CC3(CCN(c4ccc5c(c4)-c4nc(N6C(=O)OC[C@H]6C(F)F)cn4CCO5)CC3)C2)C1] | 583 |
| 23 | 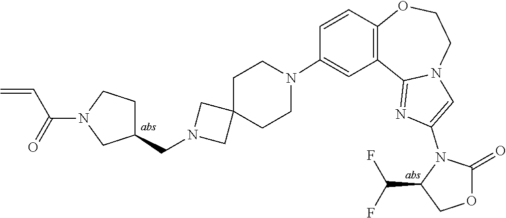 [SMILES: C=CC(=O)N1CC[C@@H](CN2CC3(CCN(c4ccc5c(c4)-c4nc(N6C(=O)OC[C@H]6C(F)F)cn4CCO5)CC3)C2)C1] | 583 |
| 24 | 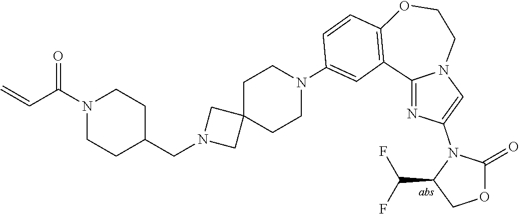 [SMILES: C=CC(=O)N1CCC(CN2CC3(CCN(c4ccc5c(c4)-c4nc(N6C(=O)OC[C@H]6C(F)F)cn4CCO5)CC3)C2)CC1] | 597 |
| 25 | 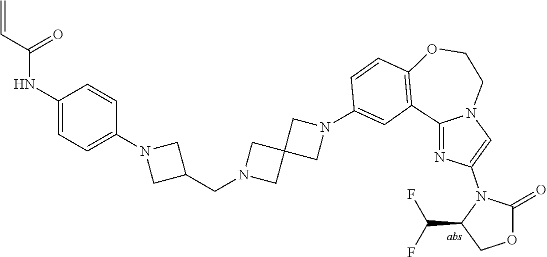 [SMILES: C=CC(=O)Nc1ccc(N2CC(CN3CC4(C3)CN(c3ccc5c(c3)-c3nc(N6C(=O)OC[C@H]6C(F)F)cn3CCO5)C4)C2)cc1] | 632 |
| 26 | 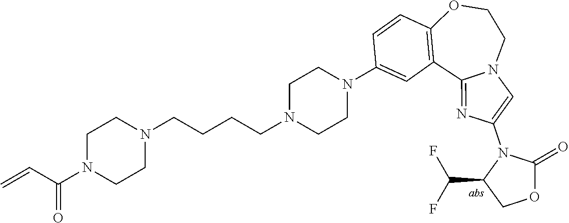 [SMILES: C=CC(=O)N1CCN(CCCCN2CCN(c3ccc4c(c3)-c3nc(N5C(=O)OC[C@H]5C(F)F)cn3CCO4)CC2)CC1] | 600 |
| 27 | 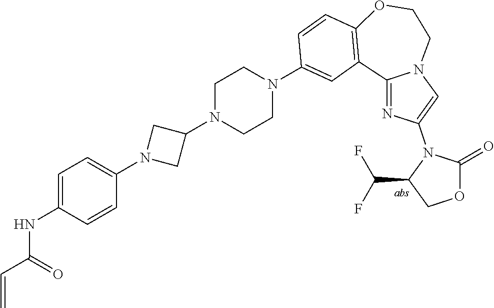 [SMILES: C=CC(=O)Nc1ccc(N2CC(N3CCN(c4ccc5c(c4)-c4nc(N6C(=O)OC[C@H]6C(F)F)cn4CCO5)CC3)C2)cc1] | 606 |
| 28 | 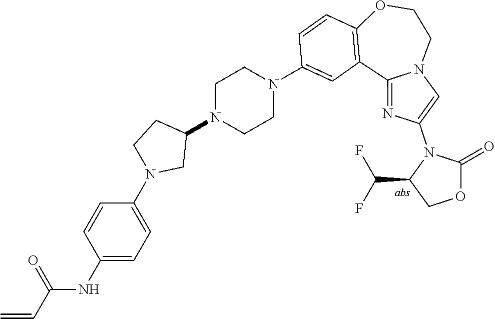 | 620 |
| | Rt = 3.561 min | |
| 29 | 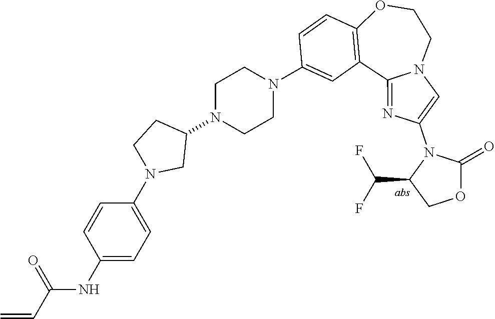 | 620 |
| | Rt = 5.252 min | |
| 30 | 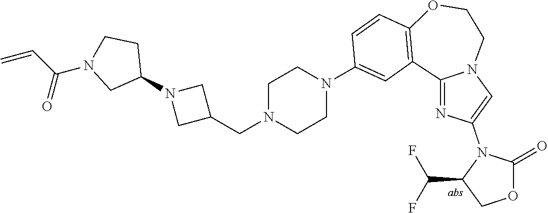 [SMILES: C=CC(=O)N1CC[C@@H](N2CC(CN3CCN(c4ccc5c(c4)-c4nc(N6C(=O)OC[C@H]6C(F)F)cn4CCO5)CC3)C2)C1] | 598 |
| 31 | 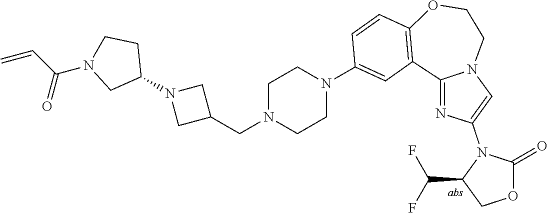 [SMILES: C=CC(=O)N1CC[C@H](N2CC(CN3CCN(c4ccc5c(c4)-c4nc(N6C(=O)OC[C@H]6C(F)F)cn4CCO5)CC3)C2)C1] | 598 |
| 32 | 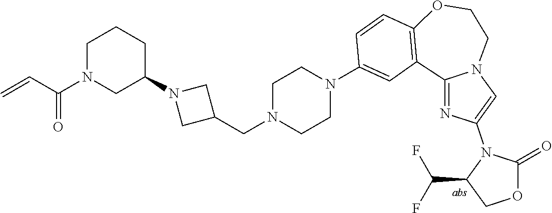 [SMILES: C=CC(=O)N1CCC[C@@H](N2CC(CN3CCN(c4ccc5c(c4)-c4nc(N6C(=O)OC[C@H]6C(F)F)cn4CCO5)CC3)C2)C1] | 612 |
| 33 | 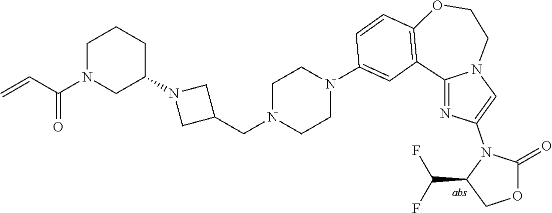 [SMILES: C=CC(=O)N1CCC[C@H](N2CC(CN3CCN(c4ccc5c(c4)-c4nc(N6C(=O)OC[C@H]6C(F)F)cn4CCO5)CC3)C2)C1] | 612 |
| 34 | 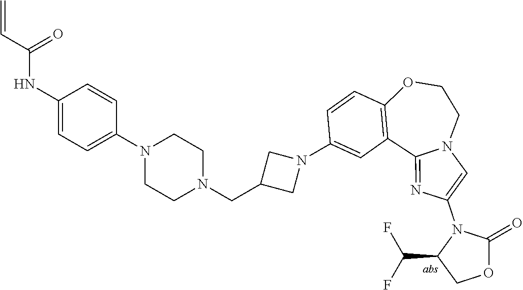 [SMILES: C=CC(=O)Nc1ccc(N2CCN(CC3CN(c4ccc5c(c4)-c4nc(N6C(=O)OC[C@H]6C(F)F)cn4CCO5)C3)CC2)cc1] | 620 |
| 35 | 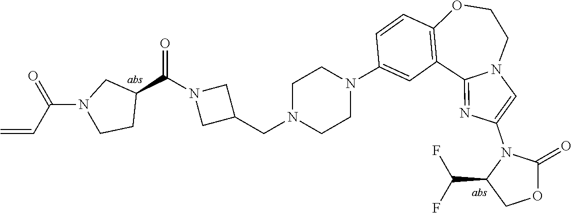 [SMILES: C=CC(=O)N1CC[C@H](C(=O)N2CC(CN3CCN(c4ccc5c(c4)-c4nc(N6C(=O)OC[C@H]6C(F)F)cn4CCO5)CC3)C2)C1] | 626 |
| 36 | 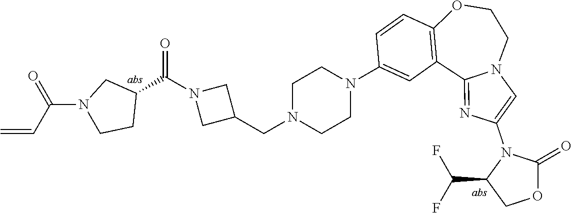 [SMILES: C=CC(=O)N1CC[C@@H](C(=O)N2CC(CN3CCN(c4ccc5c(c4)-c4nc(N6C(=O)OC[C@H]6C(F)F)cn4CCO5)CC3)C2)C1] | 626 |
| 37 | 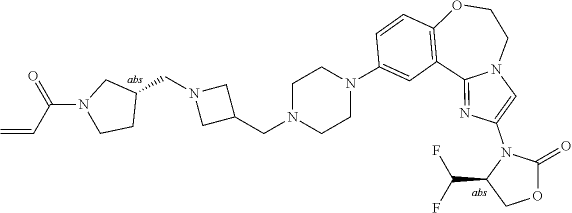 [SMILES: C=CC(=O)N1CC[C@@H](CN2CC(CN3CCN(c4ccc5c(c4)-c4nc(N6C(=O)OC[C@H]6C(F)F)cn4CCO5)CC3)C2)C1] | 612 |
| 38 | 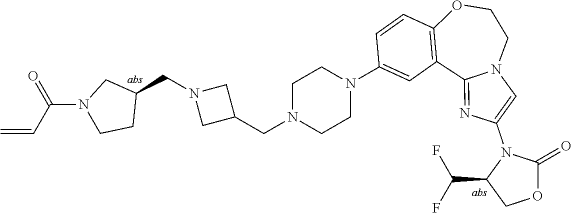 [SMILES: C=CC(=O)N1CC[C@H](CN2CC(CN3CCN(c4ccc5c(c4)-c4nc(N6C(=O)OC[C@H]6C(F)F)cn4CCO5)CC3)C2)C1] | 612 |
| 39 | 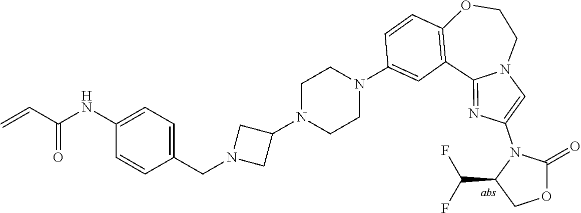 [SMILES: C=CC(=O)Nc1ccc(CN2CC(N3CCN(c4ccc5c(c4)-c4nc(N6C(=O)OC[C@H]6C(F)F)cn4CCO5)CC3)C2)cc1] | 620 |
| 40 | 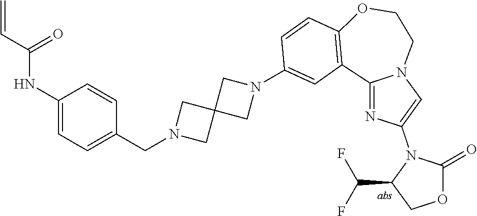 [SMILES: C=CC(=O)Nc1ccc(CN2CC3(C2)CN(c2ccc4c(c2)-c2nc(N5C(=O)OC[C@H]5C(F)F)cn2CCO4)C3)cc1] | 577 |
| 41 | 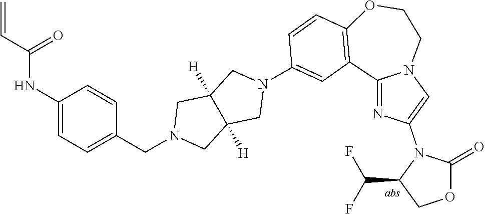 [SMILES: C=CC(=O)Nc1ccc(CN2C[C@@H]3CN(c4ccc5c(c4)-c4nc(N6C(=O)OC[C@H]6C(F)F)cn4CCO5)C[C@@H]3C2)cc1] | 591 |
| 42 | 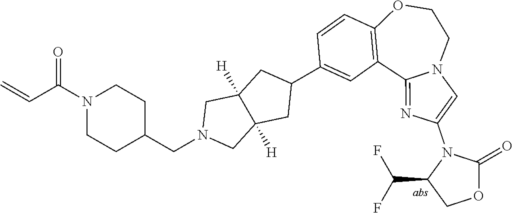 [SMILES: C=CC(=O)N1CCC(CN2C[C@H]3CC(c4ccc5c(c4)-c4nc(N6C(=O)OC[C@H]6C(F)F)cn4CCO5)C[C@H]3C2)CC1] | 582 |
| 43 | 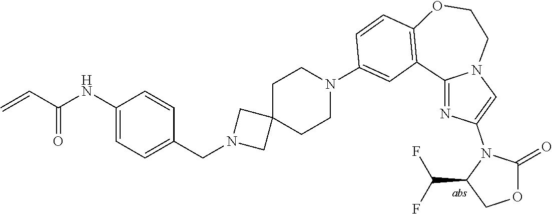 [SMILES: C=CC(=O)Nc1ccc(CN2CC3(CCN(c4ccc5c(c4)-c4nc(N6C(=O)OC[C@H]6C(F)F)cn4CCO5)CC3)C2)cc1] | 605 |
| 44 | 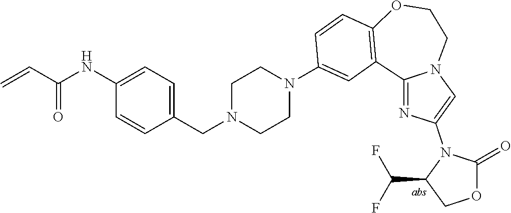 [SMILES: C=CC(=O)Nc1ccc(CN2CCN(c3ccc4c(c3)-c3nc(N5C(=O)OC[C@H]5C(F)F)cn3CCO4)CC2)cc1] | 565 |
| 45 | 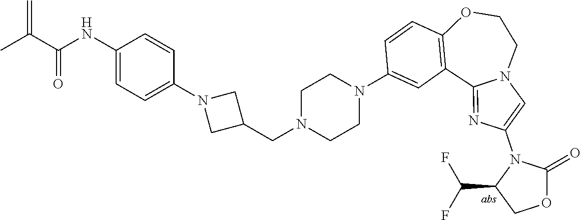 [SMILES: C=C(C)C(=O)Nc1ccc(N2CC(CN3CCN(c4ccc5c(c4)-c4nc(N6C(=O)OC[C@H]6C(F)F)cn4CCO5)CC3)C2)cc1] | 634 |
| 46 | 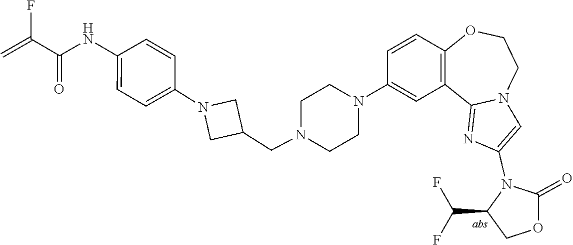 [SMILES: C=C(F)C(=O)Nc1ccc(N2CC(CN3CCN(c4ccc5c(c4)-c4nc(N6C(=O)OC[C@H]6C(F)F)cn4CCO5)CC3)C2)cc1] | 638 |
| 47 | 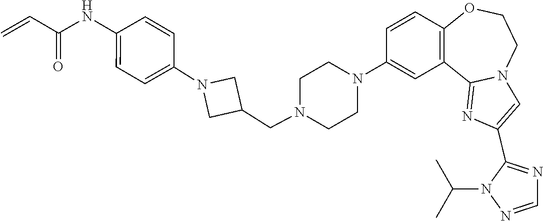 [SMILES: C=CC(=O)Nc1ccc(N2CC(CN3CCN(c4ccc5c(c4)-c4nc(-c6ncnn6C(C)C)cn4CCO5)CC3)C2)cc1] | 594 |
| 48 | 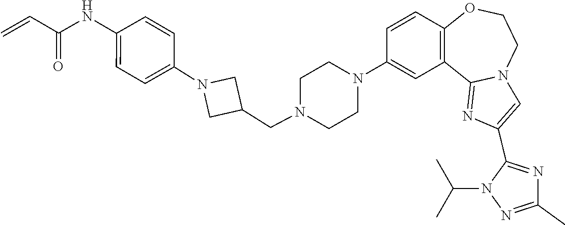 [SMILES: C=CC(=O)Nc1ccc(N2CC(CN3CCN(c4ccc5c(c4)-c4nc(-c6nc(C)nn6C(C)C)cn4CCO5)CC3)C2)cc1] | 608 |
| 49 | 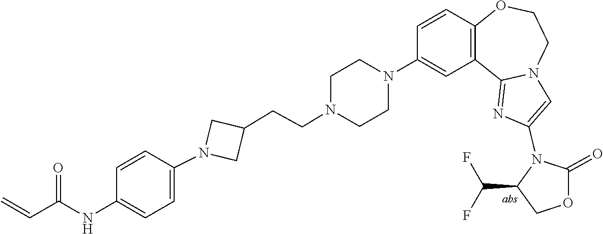 [SMILES: C=CC(=O)Nc1ccc(N2CC(CCN3CCN(c4ccc5c(c4)-c4nc(N6C(=O)OC[C@H]6C(F)F)cn4CCO5)CC3)C2)cc1] | 634 |
| 50 | 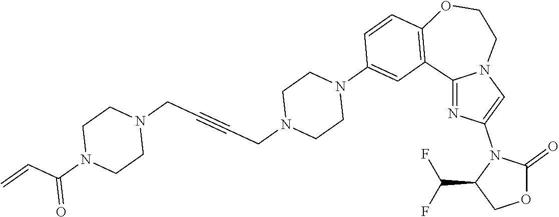 [SMILES: C=CC(=O)N1CCN(CC#CCN2CCN(c3ccc4c(c3)-c3nc(N5C(=O)OC[C@H]5C(F)F)cn3CCO4)CC2)CC1] | 596 |
| 51 | 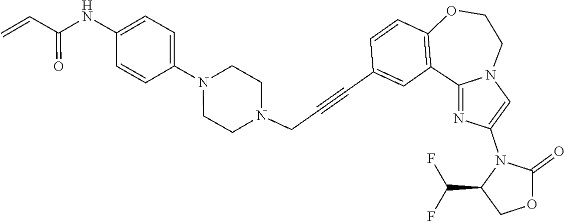 [SMILES: C=CC(=O)Nc1ccc(N2CCN(CC#Cc3ccc4c(c3)-c3nc(N5C(=O)OC[C@H]5C(F)F)cn3CCO4)CC2)cc1] | 589 |
| 52 | 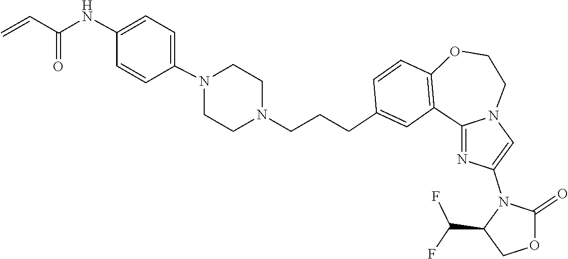 [SMILES: C=CC(=O)Nc1ccc(N2CCN(CCCc3ccc4c(c3)-c3nc(N5C(=O)OC[C@H]5C(F)F)cn3CCO4)CC2)cc1] | 593 |
| 53 | 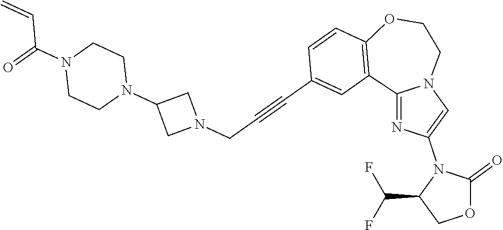 [SMILES: C=CC(=O)N1CCN(C2CN(CC#Cc3ccc4c(c3)-c3nc(N5C(=O)OC[C@H]5C(F)F)cn3CCO4)C2)CC1] | 553 |
| 54 | 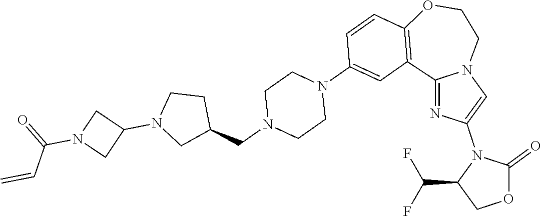 [SMILES: C=CC(=O)N1CC(N2CC[C@@H](CN3CCN(c4ccc5c(c4)-c4nc(N6C(=O)OC[C@H]6C(F)F)cn4CCO5)CC3)C2)C1] | 598 |
| 55 | 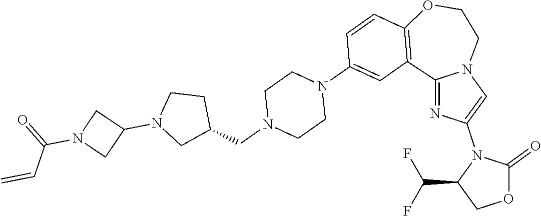 [SMILES: C=CC(=O)N1CC(N2CC[C@H](CN3CCN(c4ccc5c(c4)-c4nc(N6C(=O)OC[C@H]6C(F)F)cn4CCO5)CC3)C2)C1] | 598 |
| 56 | 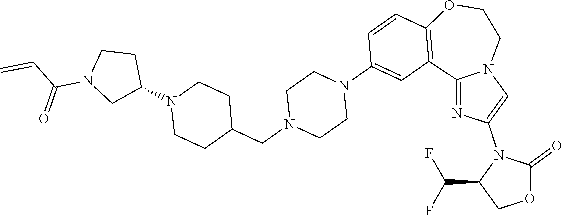 [SMILES: C=CC(=O)N1CC[C@H](N2CCC(CN3CCN(c4ccc5c(c4)-c4nc(N6C(=O)OC[C@H]6C(F)F)cn4CCO5)CC3)CC2)C1] | 626 |
| 57 | 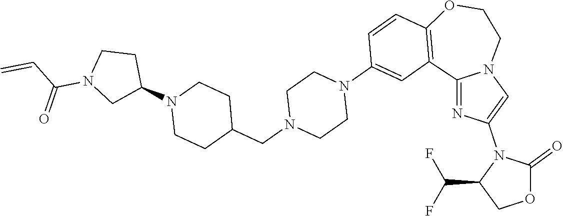 [SMILES: C=CC(=O)N1CC[C@@H](N2CCC(CN3CCN(c4ccc5c(c4)-c4nc(N6C(=O)OC[C@H]6C(F)F)cn4CCO5)CC3)CC2)C1] | 626 |
| 58 | 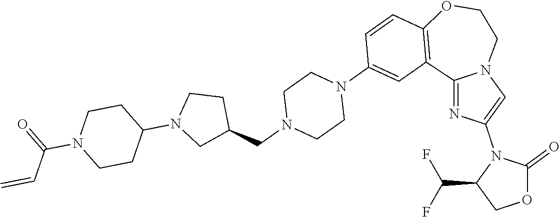 [SMILES: C=CC(=O)N1CCC(N2CC[C@@H](CN3CCN(c4ccc5c(c4)-c4nc(N6C(=O)OC[C@H]6C(F)F)cn4CCO5)CC3)C2)CC1] | 626 |
| 59 | 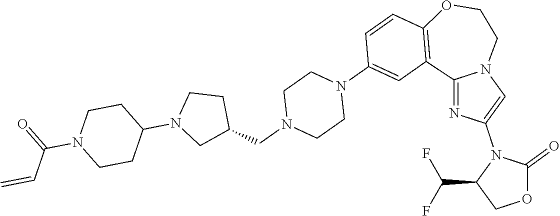 [SMILES: C=CC(=O)N1CCC(N2CC[C@H](CN3CCN(c4ccc5c(c4)-c4nc(N6C(=O)OC[C@H]6C(F)F)cn4CCO5)CC3)C2)CC1] | 626 |
| 60 | 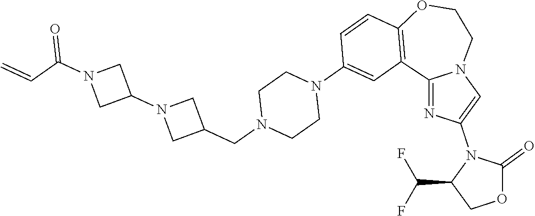 [SMILES: C=CC(=O)N1CC(N2CC(CN3CCN(c4ccc5c(c4)-c4nc(N6C(=O)OC[C@H]6C(F)F)cn4CCO5)CC3)C2)C1] | 584 |
| 61 | 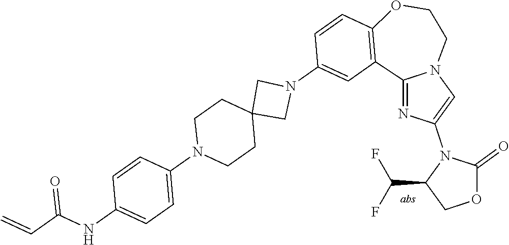 [SMILES: C=CC(=O)Nc1ccc(N2CCC3(CC2)CN(c2ccc4c(c2)-c2nc(N5C(=O)OC[C@H]5C(F)F)cn2CCO4)C3)cc1] | 591 |
| 62 | 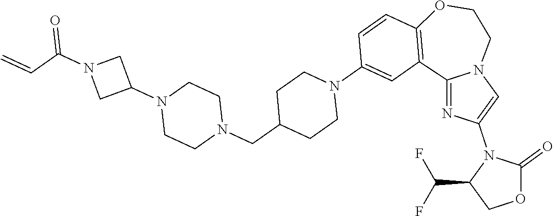 [SMILES: C=CC(=O)N1CC(N2CCN(CC3CCN(c4ccc5c(c4)-c4nc(N6C(=O)OC[C@H]6C(F)F)cn4CCO5)CC3)CC2)C1] | 612 |
| 63 | 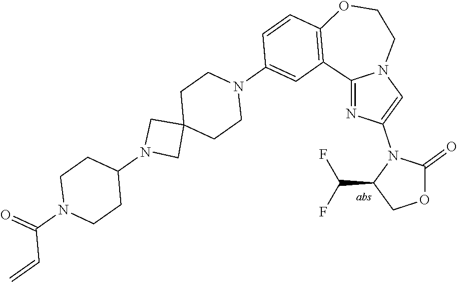 [SMILES: C=CC(=O)N1CCC(N2CC3(CCN(c4ccc5c(c4)-c4nc(N6C(=O)OC[C@H]6C(F)F)cn4CCO5)CC3)C2)CC1] | 583 |
| 64 | 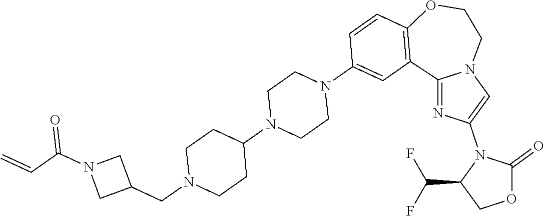 [SMILES: C=CC(=O)N1CC(CN2CCC(N3CCN(c4ccc5c(c4)-c4nc(N6C(=O)OC[C@H]6C(F)F)cn4CCO5)CC3)CC2)C1] | 612 |
| 65 | 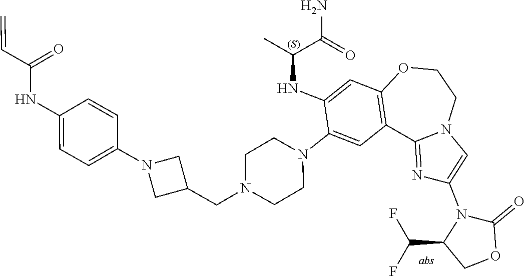 | 706 |
| 66 | 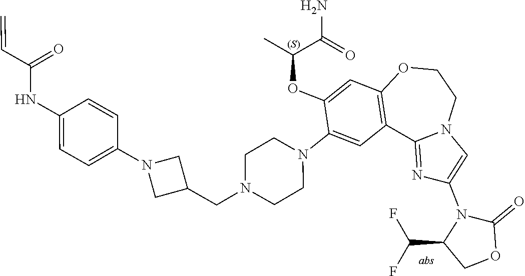 | 707 |
| 67 | 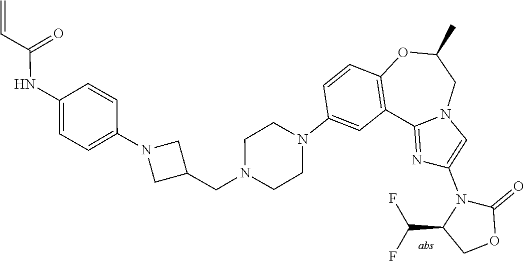 [SMILES: C=CC(=O)Nc1ccc(N2CC(CN3CCN(c4ccc5c(c4)-c4nc(N6C(=O)OC[C@H]6C(F)F)cn4C[C@H](C)O5)CC3)C2)cc1] | 634 |
| 68 | 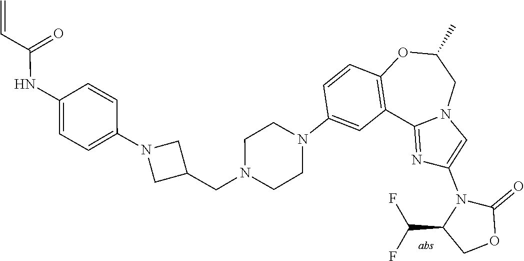 [SMILES: C=CC(=O)Nc1ccc(N2CC(CN3CCN(c4ccc5c(c4)-c4nc(N6C(=O)OC[C@H]6C(F)F)cn4C[C@@H](C)O5)CC3)C2)cc1] | 634 |
| 69 | 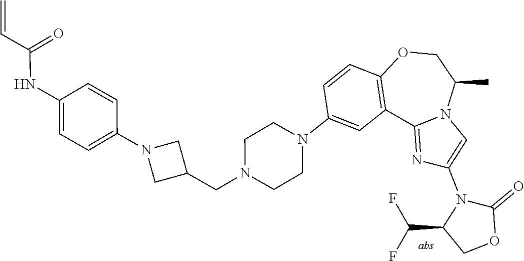 [SMILES: C=CC(=O)Nc1ccc(N2CC(CN3CCN(c4ccc5c(c4)-c4nc(N6C(=O)OC[C@H]6C(F)F)cn4[C@H](C)CO5)CC3)C2)cc1] | 634 |
| 70 | 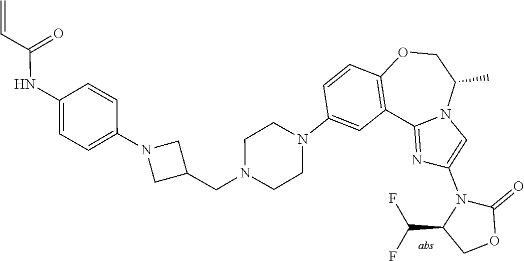 [SMILES: C=CC(=O)Nc1ccc(N2CC(CN3CCN(c4ccc5c(c4)-c4nc(N6C(=O)OC[C@H]6C(F)F)cn4[C@@H](C)CO5)CC3)C2)cc1] | 634 |
| 71 | 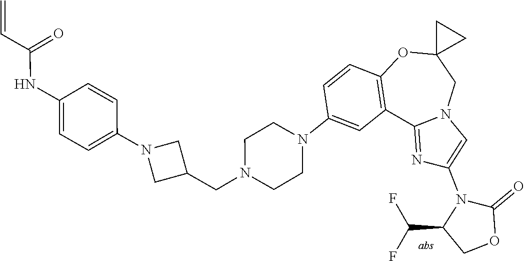 [SMILES: C=CC(=O)Nc1ccc(N2CC(CN3CCN(c4ccc5c(c4)-c4nc(N6C(=O)OC[C@H]6C(F)F)cn4CC4(CC4)O5)CC3)C2)cc1] | 646 |
| 72 | 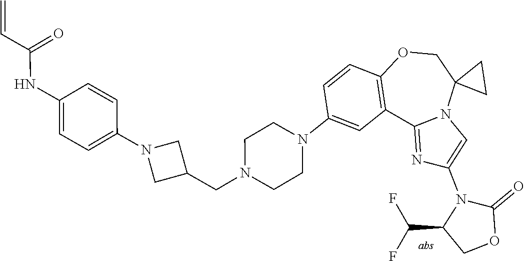 [SMILES: C=CC(=O)Nc1ccc(N2CC(CN3CCN(c4ccc5c(c4)-c4nc(N6C(=O)OC[C@H]6C(F)F)cn4C4(CC4)CO5)CC3)C2)cc1] | 646 |
| 73 | 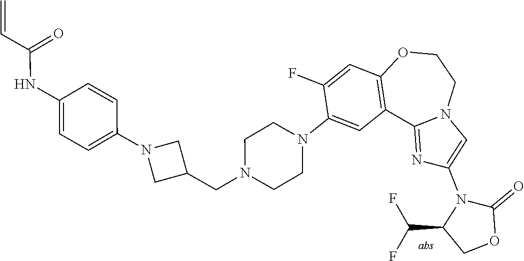 [SMILES: C=CC(=O)Nc1ccc(N2CC(CN3CCN(c4cc5c(cc4F)OCCn4cc(N6C(=O)OC[C@H]6C(F)F)nc4-5)CC3)C2)cc1] | 638 |
| 74 | 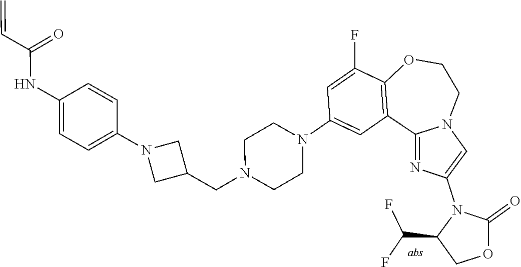 [SMILES: C=CC(=O)Nc1ccc(N2CC(CN3CCN(c4cc(F)c5c(c4)-c4nc(N6C(=O)OC[C@H]6C(F)F)cn4CCO5)CC3)C2)cc1] | 638 |
| 75 | 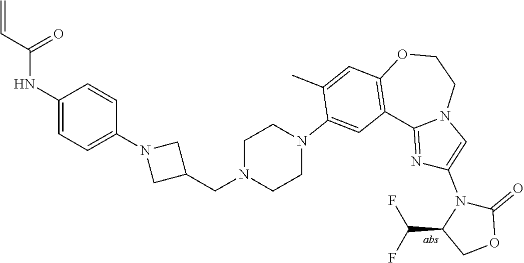 [SMILES: C=CC(=O)Nc1ccc(N2CC(CN3CCN(c4cc5c(cc4C)OCCn4cc(N6C(=O)OC[C@H]6C(F)F)nc4-5)CC3)C2)cc1] | 634 |
| 76 | 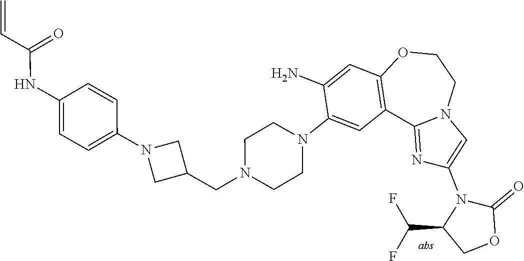 [SMILES: C=CC(=O)Nc1ccc(N2CC(CN3CCN(c4cc5c(cc4N)OCCn4cc(N6C(=O)OC[C@H]6C(F)F)nc4-5)CC3)C2)cc1] | 635 |
| 77 | 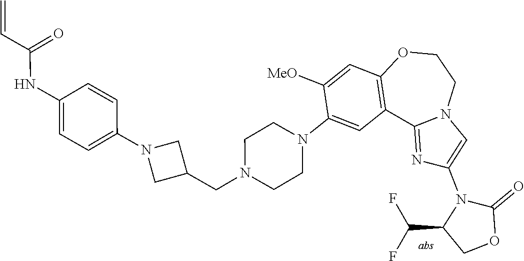 [SMILES: C=CC(=O)Nc1ccc(N2CC(CN3CCN(c4cc5c(cc4OC)OCCn4cc(N6C(=O)OC[C@H]6C(F)F)nc4-5)CC3)C2)cc1] | 650 |
| 78 | 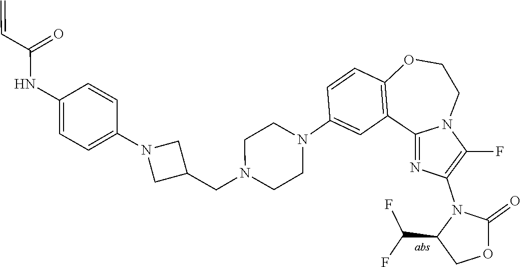 [SMILES: C=CC(=O)Nc1ccc(N2CC(CN3CCN(c4ccc5c(c4)-c4nc(N6C(=O)OC[C@H]6C(F)F)c(F)n4CCO5)CC3)C2)cc1] | 638 |
| 79 | 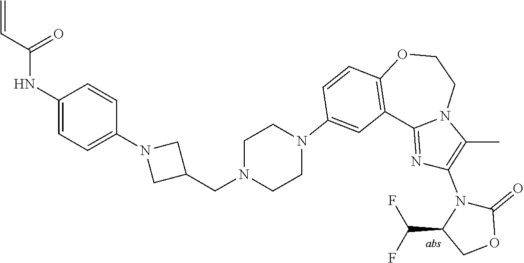 [SMILES: C=CC(=O)Nc1ccc(N2CC(CN3CCN(c4ccc5c(c4)-c4nc(N6C(=O)OC[C@H]6C(F)F)c(C)n4CCO5)CC3)C2)cc1] | 634 |
| 80 | 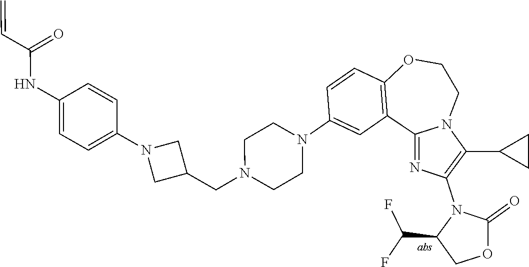 [SMILES: C=CC(=O)Nc1ccc(N2CC(CN3CCN(c4ccc5c(c4)-c4nc(N6C(=O)OC[C@H]6C(F)F)c(C6CC6)n4CCO5)CC3)C2)cc1] | 660 |
| 81 | 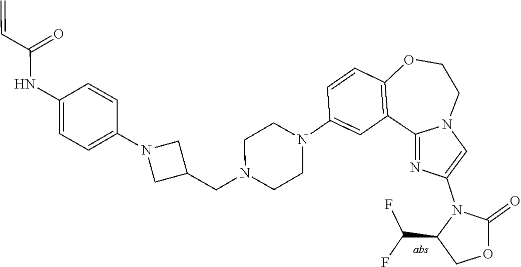 [SMILES: C=CC(=O)Nc1ccc(N2CC(CN3CCN(c4ccc5c(c4)-c4nc(N6C(=O)OC[C@H]6C(F)F)cn4CCO5)CC3)C2)cc1] | 636 |
| 82 | 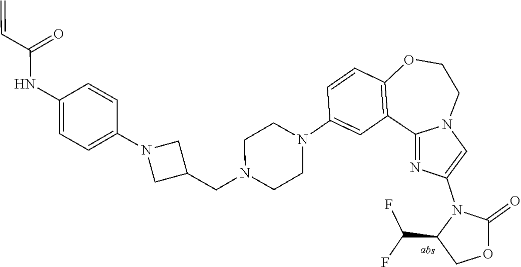 [SMILES: C=CC(=O)Nc1ccc(N2CC(CN3CCN(c4ccc5c(c4)-c4nc(N6C(=O)OC[C@H]6C(F)F)cn4CCO5)CC3)C2)cc1] | 636 |
| 83 | 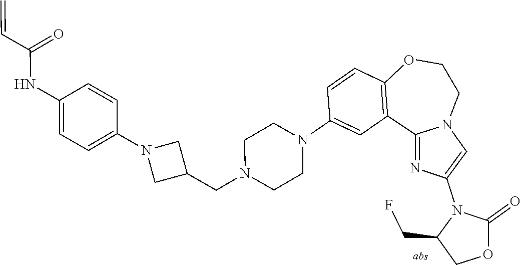 [SMILES: C=CC(=O)Nc1ccc(N2CC(CN3CCN(c4ccc5c(c4)-c4nc(N6C(=O)OC[C@H]6CF)cn4CCO5)CC3)C2)cc1] | 602 |
| 84 | 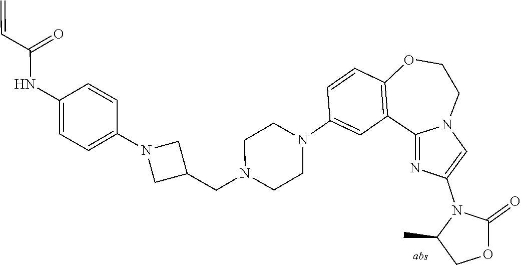 [SMILES: C=CC(=O)Nc1ccc(N2CC(CN3CCN(c4ccc5c(c4)-c4nc(N6C(=O)OC[C@H]6C)cn4CCO5)CC3)C2)cc1] | 584 |
| 85 | 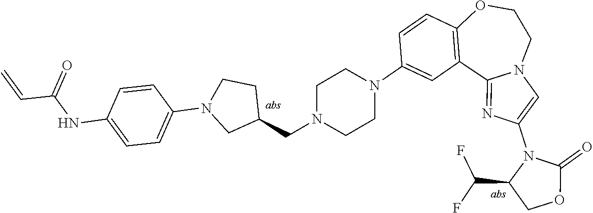 [SMILES: C=CC(=O)Nc1ccc(N2CC[C@@H](CN3CCN(c4ccc5c(c4)-c4nc(N6C(=O)OC[C@H]6C(F)F)cn4CCO5)CC3)C2)cc1] | 634 |
| 86 | 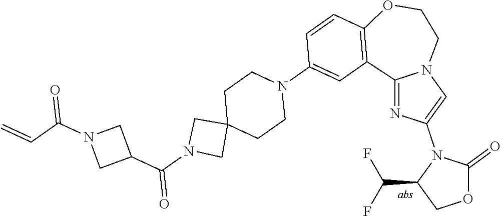 [SMILES: C=CC(=O)N1CC(C(=O)N2CC3(CCN(c4ccc5c(c4)-c4nc(N6C(=O)OC[C@H]6C(F)F)cn4CCO5)CC3)C2)C1] | 583 |
| 87 | 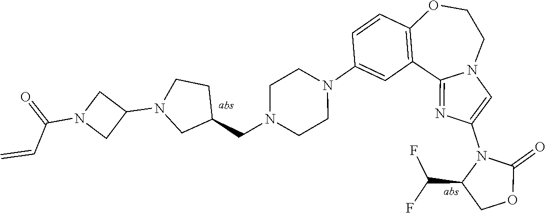 [SMILES: C=CC(=O)N1CC(N2CC[C@@H](CN3CCN(c4ccc5c(c4)-c4nc(N6C(=O)OC[C@H]6C(F)F)cn4CCO5)CC3)C2)C1] | 598 |
| 88 | 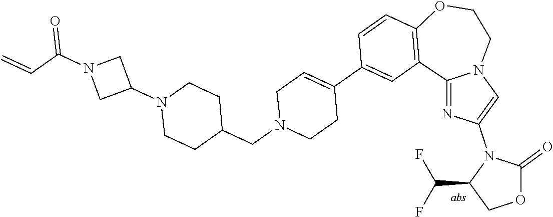 [SMILES: C=CC(=O)N1CC(N2CCC(CN3CC=C(c4ccc5c(c4)-c4nc(N6C(=O)OC[C@H]6C(F)F)cn4CCO5)CC3)CC2)C1] | 609 |
| 89 | 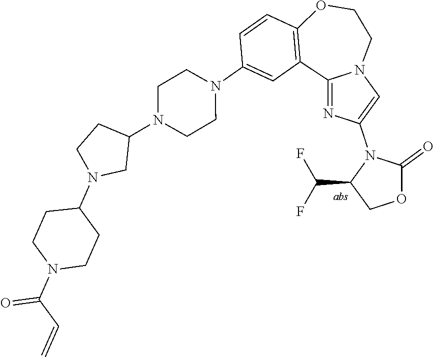 [SMILES: C=CC(=O)N1CCC(N2CCC(N3CCN(c4ccc5c(c4)-c4nc(N6C(=O)OC[C@H]6C(F)F)cn4CCO5)CC3)C2)CC1] | 612 |
| 90 | 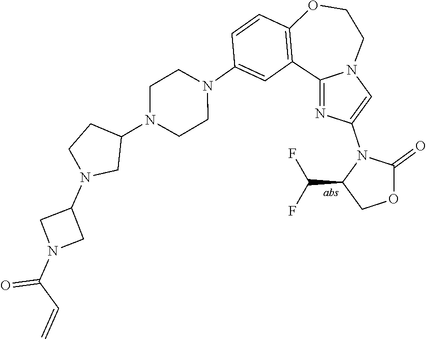 [SMILES: C=CC(=O)N1CC(N2CCC(N3CCN(c4ccc5c(c4)-c4nc(N6C(=O)OC[C@H]6C(F)F)cn4CCO5)CC3)C2)C1] | 584 |
| 91 | 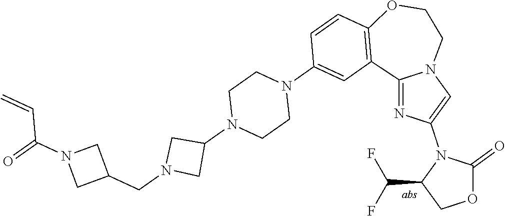 [SMILES: C=CC(=O)N1CC(CN2CC(N3CCN(c4ccc5c(c4)-c4nc(N6C(=O)OC[C@H]6C(F)F)cn4CCO5)CC3)C2)C1] | 584 |
| 92 | 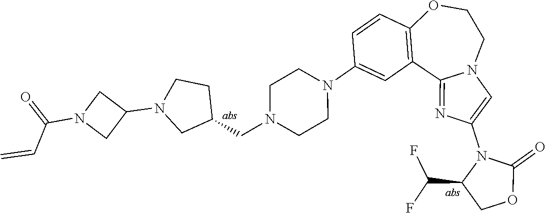 [SMILES: C=CC(=O)N1CC(N2CC[C@H](CN3CCN(c4ccc5c(c4)-c4nc(N6C(=O)OC[C@H]6C(F)F)cn4CCO5)CC3)C2)C1] | 598 |
| 93 | 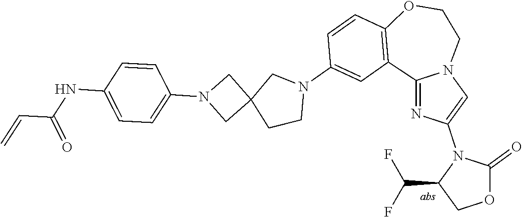 [SMILES: C=CC(=O)Nc1ccc(N2CC3(CCN(c4ccc5c(c4)-c4nc(N6C(=O)OC[C@H]6C(F)F)cn4CCO5)C3)C2)cc1] | 577 |
| 94 | 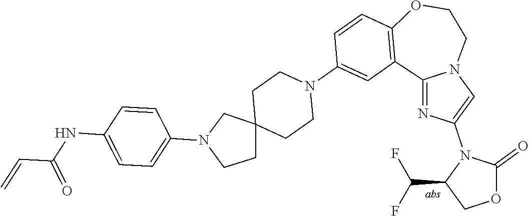 [SMILES: C=CC(=O)Nc1ccc(N2CCC3(CCN(c4ccc5c(c4)-c4nc(N6C(=O)OC[C@H]6C(F)F)cn4CCO5)CC3)C2)cc1] | 605 |
| 96 | 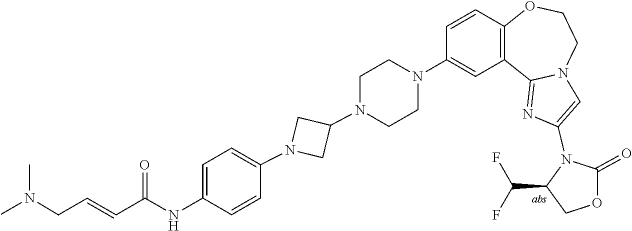 [SMILES: CN(C)C/C=C/C(=O)Nc1ccc(N2CC(N3CCN(c4ccc5c(c4)-c4nc(N6C(=O)OC[C@H]6C(F)F)cn4CCO5)CC3)C2)cc1] | 663 |
| 97 | 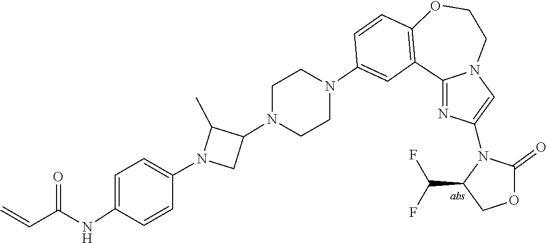 [SMILES: C=CC(=O)Nc1ccc(N2CC(N3CCN(c4ccc5c(c4)-c4nc(N6C(=O)OC[C@H]6C(F)F)cn4CCO5)CC3)C2C)cc1] | 619 |
| 98 | 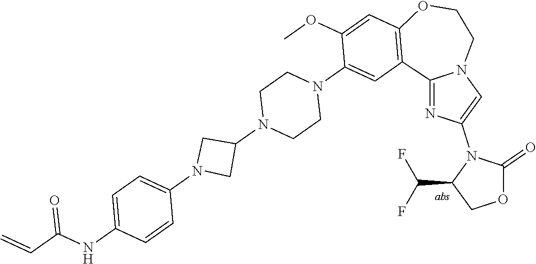 [SMILES: C=CC(=O)Nc1ccc(N2CC(N3CCN(c4cc5c(cc4OC)OCCn4cc(N6C(=O)OC[C@H]6C(F)F)nc4-5)CC3)C2)cc1] | 636 |
| 99 | 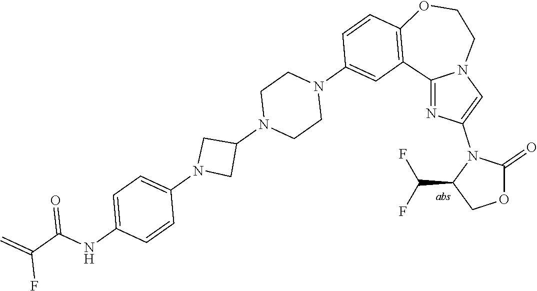 [SMILES: C=C(F)C(=O)Nc1ccc(N2CC(N3CCN(c4ccc5c(c4)-c4nc(N6C(=O)OC[C@H]6C(F)F)cn4CCO5)CC3)C2)cc1] | 624 |
| 100 | 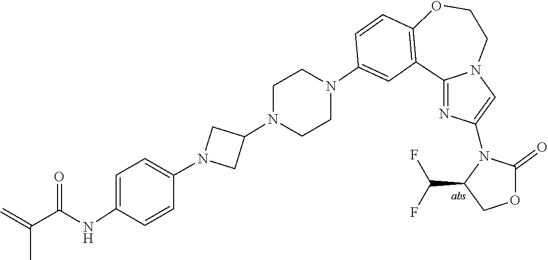 [SMILES: C=C(C)C(=O)Nc1ccc(N2CC(N3CCN(c4ccc5c(c4)-c4nc(N6C(=O)OC[C@H]6C(F)F)cn4CCO5)CC3)C2)cc1] | 620 |
| 101 | 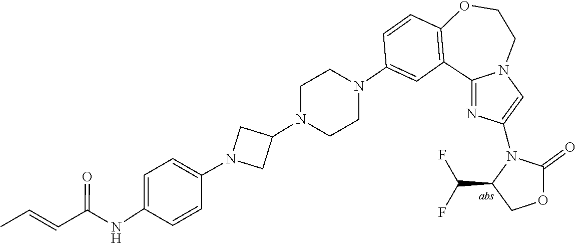 [SMILES: C/C=C/C(=O)Nc1ccc(N2CC(N3CCN(c4ccc5c(c4)-c4nc(N6C(=O)OC[C@H]6C(F)F)cn4CCO5)CC3)C2)cc1] | 620 |
| 102 | 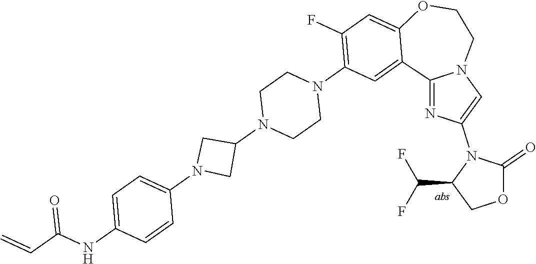 [SMILES: C=CC(=O)Nc1ccc(N2CC(N3CCN(c4cc5c(cc4F)OCCn4cc(N6C(=O)OC[C@H]6C(F)F)nc4-5)CC3)C2)cc1] | 624 |
| 103 | 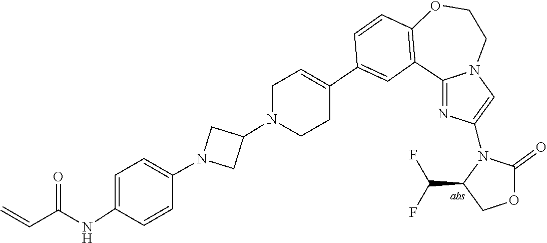 [SMILES: C=CC(=O)Nc1ccc(N2CC(N3CC=C(c4ccc5c(c4)-c4nc(N6C(=O)OC[C@H]6C(F)F)cn4CCO5)CC3)C2)cc1] | 603 |
| 104 | 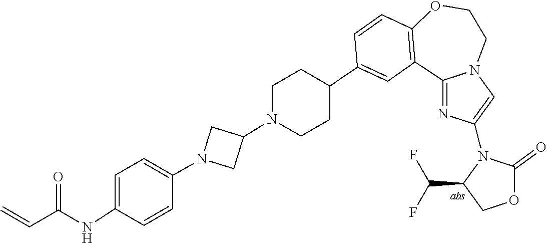 [SMILES: C=CC(=O)Nc1ccc(N2CC(N3CCC(c4ccc5c(c4)-c4nc(N6C(=O)OC[C@H]6C(F)F)cn4CCO5)CC3)C2)cc1] | 605 |
| 105 | 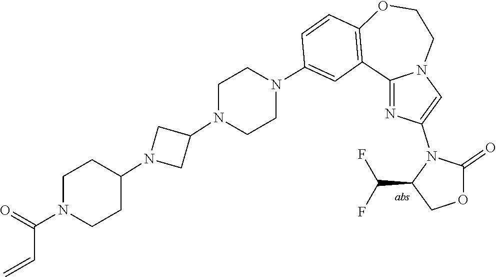 [SMILES: C=CC(=O)N1CCC(N2CC(N3CCN(c4ccc5c(c4)-c4nc(N6C(=O)OC[C@H]6C(F)F)cn4CCO5)CC3)C2)CC1] | 598 |
| 107 | 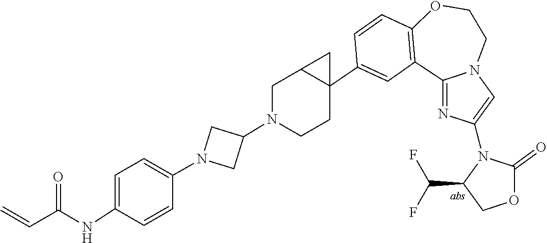 [SMILES: C=CC(=O)Nc1ccc(N2CC(N3CCC4(c5ccc6c(c5)-c5nc(N7C(=O)OC[C@H]7C(F)F)cn5CCO6)CC4C3)C2)cc1] | 617 |
| 108 | 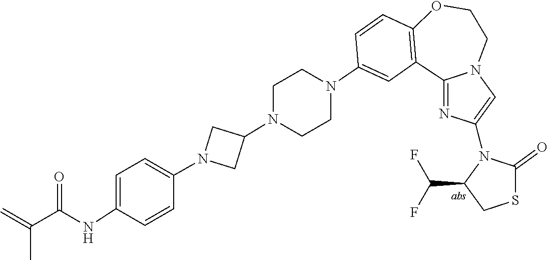 [SMILES: C=C(C)C(=O)Nc1ccc(N2CC(N3CCN(c4ccc5c(c4)-c4nc(N6C(=O)SC[C@H]6C(F)F)cn4CCO5)CC3)C2)cc1] | 636 |
| 109 | 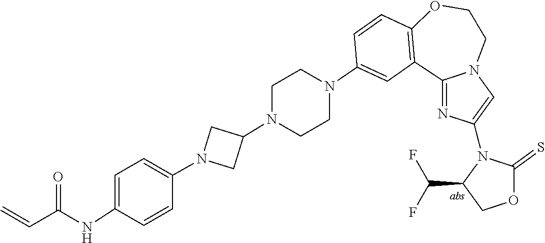 [SMILES: C=CC(=O)Nc1ccc(N2CC(N3CCN(c4ccc5c(c4)-c4nc(N6C(=S)OC[C@H]6C(F)F)cn4CCO5)CC3)C2)cc1] | 622 |
| 110 | 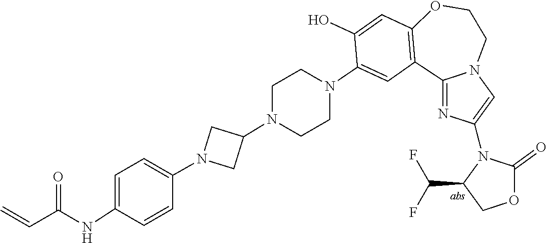 [SMILES: C=CC(=O)Nc1ccc(N2CC(N3CCN(c4cc5c(cc4O)OCCn4cc(N6C(=O)OC[C@H]6C(F)F)nc4-5)CC3)C2)cc1] | 622 |
| 111 | 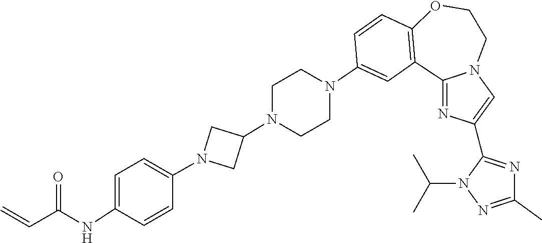 [SMILES: C=CC(=O)Nc1ccc(N2CC(N3CCN(c4ccc5c(c4)-c4nc(-c6nc(C)nn6C(C)C)cn4CCO5)CC3)C2)cc1] | 594 |
| 112 | 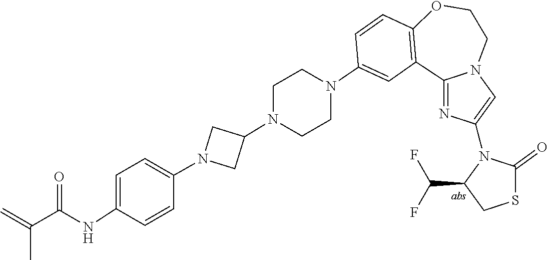 [SMILES: C=C(C)C(=O)Nc1ccc(N2CC(N3CCN(c4ccc5c(c4)-c4nc(N6C(=O)SC[C@H]6C(F)F)cn4CCO5)CC3)C2)cc1] | 636 |
| 113 | 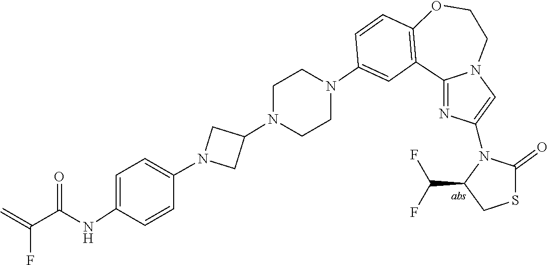 [SMILES: C=C(F)C(=O)Nc1ccc(N2CC(N3CCN(c4ccc5c(c4)-c4nc(N6C(=O)SC[C@H]6C(F)F)cn4CCO5)CC3)C2)cc1] | 640 |
| 114 | 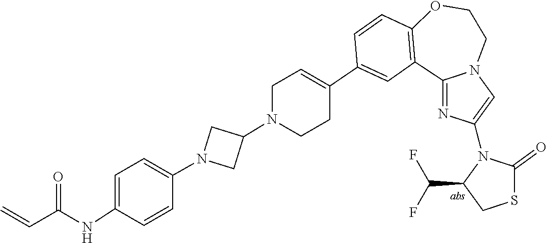 [SMILES: C=CC(=O)Nc1ccc(N2CC(N3CC=C(c4ccc5c(c4)-c4nc(N6C(=O)SC[C@H]6C(F)F)cn4CCO5)CC3)C2)cc1] | 619 |
| 115 | 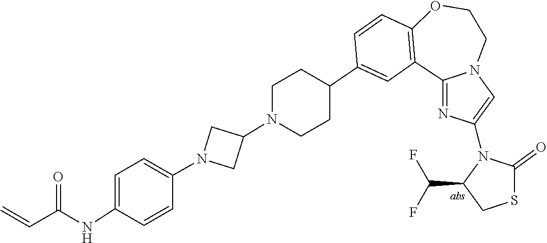 [SMILES: C=CC(=O)Nc1ccc(N2CC(N3CCC(c4ccc5c(c4)-c4nc(N6C(=O)SC[C@H]6C(F)F)cn4CCO5)CC3)C2)cc1] | 621 |
| 116 | 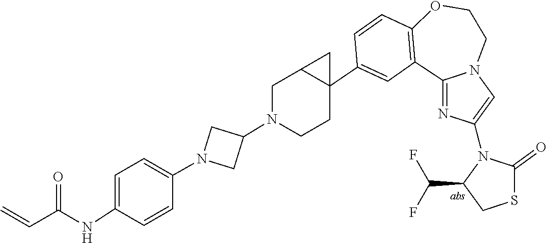 [SMILES: C=CC(=O)Nc1ccc(N2CC(N3CCC4(c5ccc6c(c5)-c5nc(N7C(=O)SC[C@H]7C(F)F)cn5CCO6)CC4C3)C2)cc1] | 633 |
| 117 | 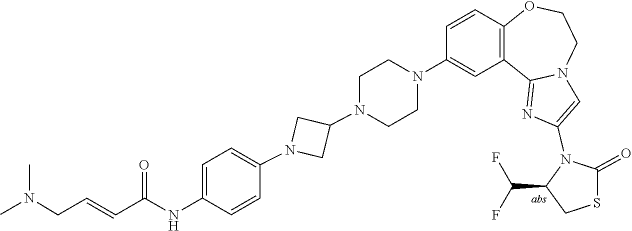 [SMILES: CN(C)C/C=C/C(=O)Nc1ccc(N2CC(N3CCN(c4ccc5c(c4)-c4nc(N6C(=O)SC[C@H]6C(F)F)cn4CCO5)CC3)C2)cc1] | 679 |
| 118 | 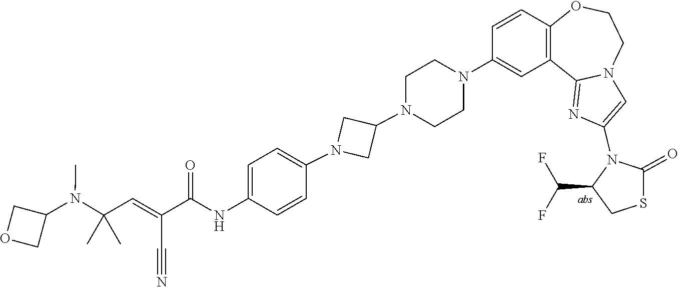 [SMILES: CN(C1COC1)C(C)(C)/C=C(\C#N)C(=O)Nc1ccc(N2CC(N3CCN(c4ccc5c(c4)-c4nc(N6C(=O)SC[C@H]6C(F)F)cn4CCO5)CC3)C2)cc1] | 774 |
| 119 | 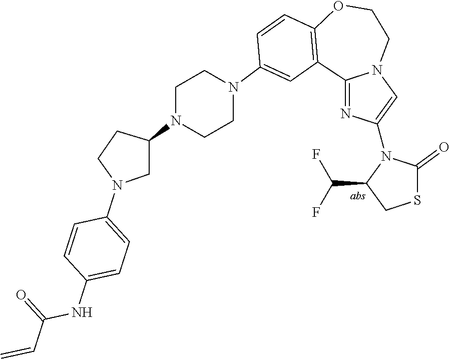 [SMILES: C=CC(=O)Nc1ccc(N2CC[C@@H](N3CCN(c4ccc5c(c4)-c4nc(N6C(=O)SC[C@H]6C(F)F)cn4CCO5)CC3)C2)cc1] | 636 |
| 120 | 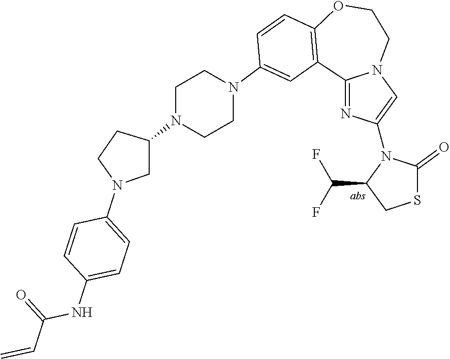 [SMILES: C=CC(=O)Nc1ccc(N2CC[C@H](N3CCN(c4ccc5c(c4)-c4nc(N6C(=O)SC[C@H]6C(F)F)cn4CCO5)CC3)C2)cc1] | 636 |
| 121 | 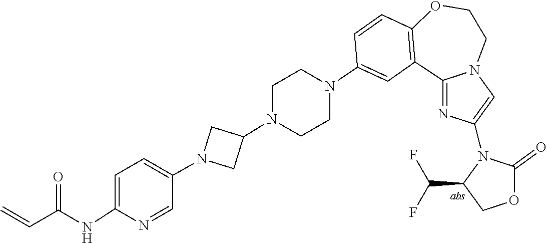 [SMILES: C=CC(=O)Nc1ccc(N2CC(N3CCN(c4ccc5c(c4)-c4nc(N6C(=O)OC[C@H]6C(F)F)cn4CCO5)CC3)C2)cn1] | 607 |
| 122 | 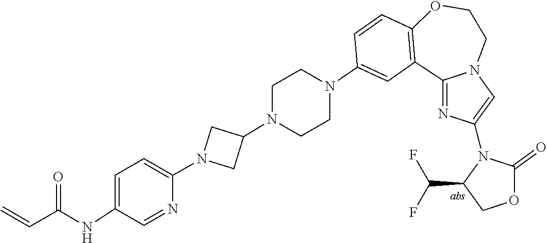 [SMILES: C=CC(=O)Nc1ccc(N2CC(N3CCN(c4ccc5c(c4)-c4nc(N6C(=O)OC[C@H]6C(F)F)cn4CCO5)CC3)C2)nc1] | 607 |
| 123 | 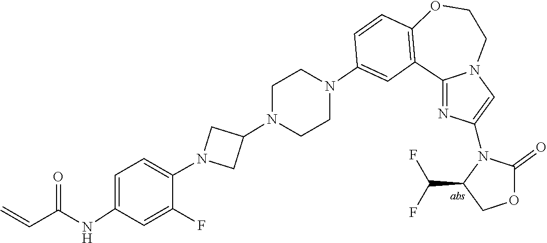 [SMILES: C=CC(=O)Nc1ccc(N2CC(N3CCN(c4ccc5c(c4)-c4nc(N6C(=O)OC[C@H]6C(F)F)cn4CCO5)CC3)C2)c(F)c1] | 624 |
| 124 | 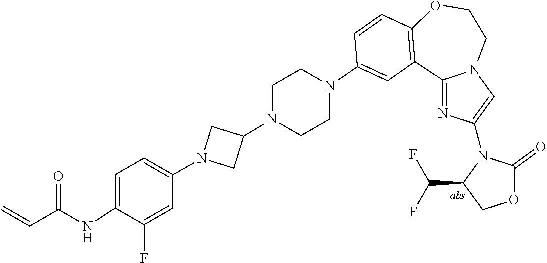 [SMILES: C=CC(=O)Nc1ccc(N2CC(N3CCN(c4ccc5c(c4)-c4nc(N6C(=O)OC[C@H]6C(F)F)cn4CCO5)CC3)C2)cc1F] | 624 |
| 125 | 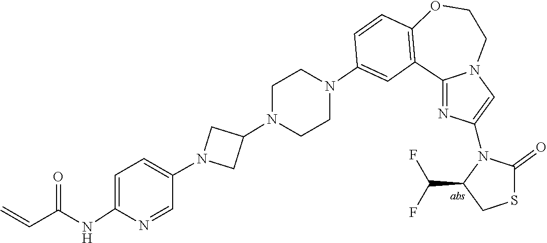 [SMILES: C=CC(=O)Nc1ccc(N2CC(N3CCN(c4ccc5c(c4)-c4nc(N6C(=O)SC[C@H]6C(F)F)cn4CCO5)CC3)C2)cn1] | 623 |
| 126 | 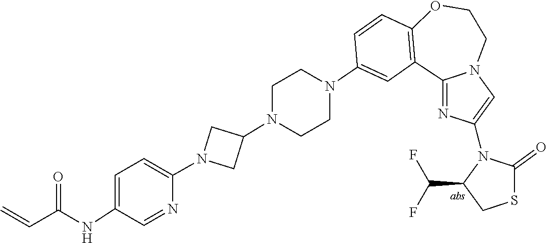 [SMILES: C=CC(=O)Nc1ccc(N2CC(N3CCN(c4ccc5c(c4)-c4nc(N6C(=O)SC[C@H]6C(F)F)cn4CCO5)CC3)C2)nc1] | 623 |
| 127 | 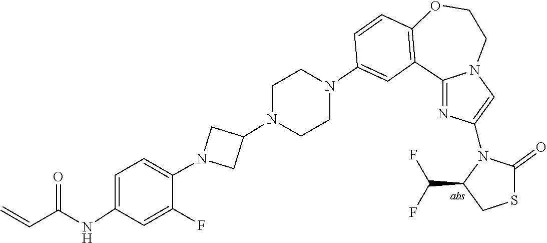 [SMILES: C=CC(=O)Nc1ccc(N2CC(N3CCN(c4ccc5c(c4)-c4nc(N6C(=O)SC[C@H]6C(F)F)cn4CCO5)CC3)C2)c(F)c1] | 640 |
| 128 | 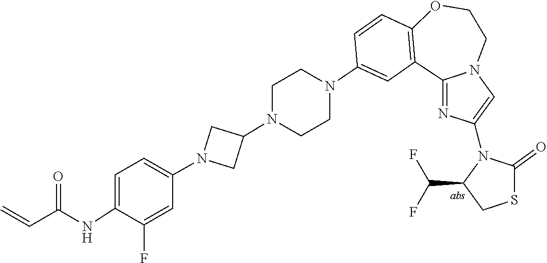 [SMILES: C=CC(=O)Nc1ccc(N2CC(N3CCN(c4ccc5c(c4)-c4nc(N6C(=O)SC[C@H]6C(F)F)cn4CCO5)CC3)C2)cc1F] | 640 |
| 129 | 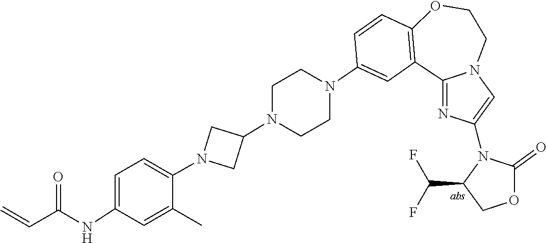 [SMILES: C=CC(=O)Nc1ccc(N2CC(N3CCN(c4ccc5c(c4)-c4nc(N6C(=O)OC[C@H]6C(F)F)cn4CCO5)CC3)C2)c(C)c1] | 620 |
| 130 | 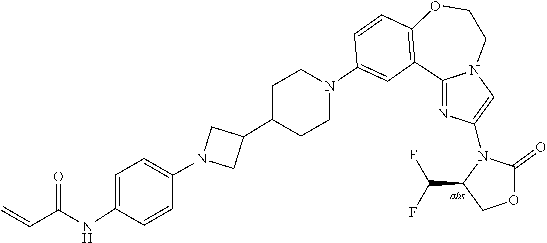 [SMILES: C=CC(=O)Nc1ccc(N2CC(C3CCN(c4ccc5c(c4)-c4nc(N6C(=O)OC[C@H]6C(F)F)cn4CCO5)CC3)C2)cc1] | 605 |
| 131 | 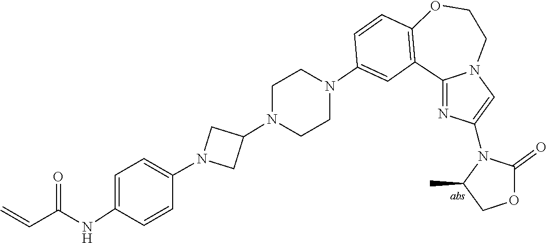 [SMILES: C=CC(=O)Nc1ccc(N2CC(N3CCN(c4ccc5c(c4)-c4nc(N6C(=O)OC[C@H]6C)cn4CCO5)CC3)C2)cc1] | 570 |
| 132 | 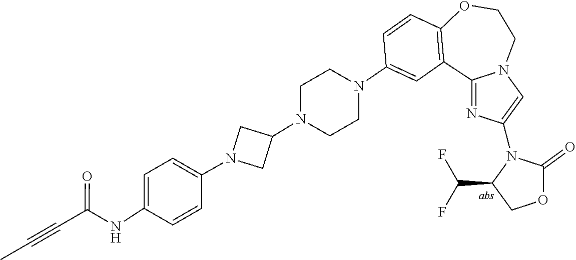 [SMILES: CC#CC(=O)Nc1ccc(N2CC(N3CCN(c4ccc5c(c4)-c4nc(N6C(=O)OC[C@H]6C(F)F)cn4CCO5)CC3)C2)cc1] | 618 |
| 133 | 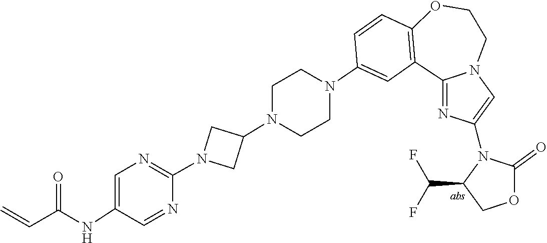 [SMILES: C=CC(=O)Nc1cnc(N2CC(N3CCN(c4ccc5c(c4)-c4nc(N6C(=O)OC[C@H]6C(F)F)cn4CCO5)CC3)C2)nc1] | 608 |
| 134 | 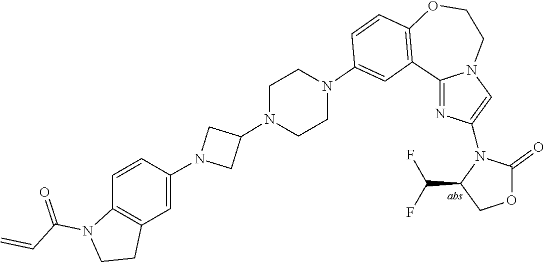 [SMILES: C=CC(=O)N1CCc2cc(N3CC(N4CCN(c5ccc6c(c5)-c5nc(N7C(=O)OC[C@H]7C(F)F)cn5CCO6)CC4)C3)ccc21] | 632 |
| 135 | 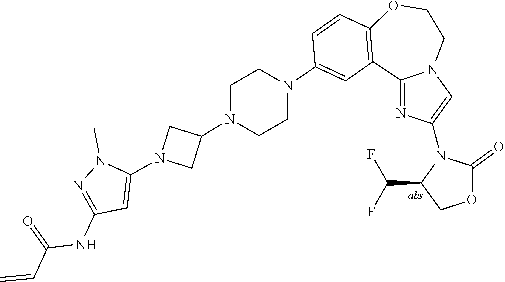 [SMILES: C=CC(=O)Nc1cc(N2CC(N3CCN(c4ccc5c(c4)-c4nc(N6C(=O)OC[C@H]6C(F)F)cn4CCO5)CC3)C2)n(C)n1] | 610 |
| 136 | 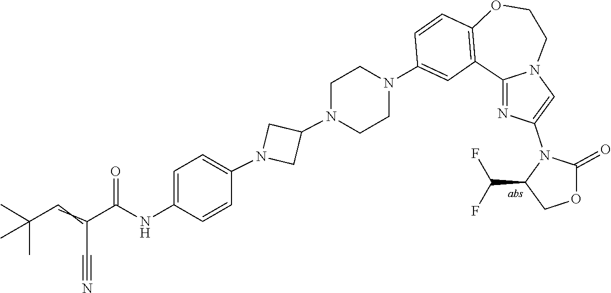 [SMILES: CC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1] | 687 |
| 137 | 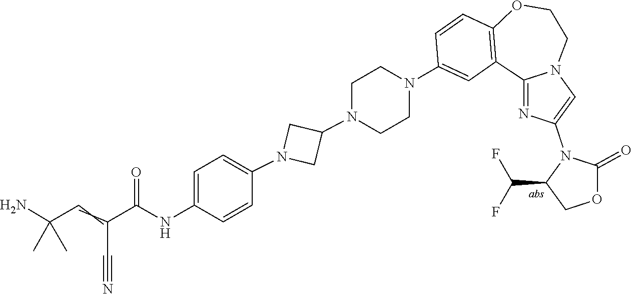 [SMILES: CC(C)(N)C=C(C#N)C(=O)Nc1ccc(N2CC(N3CCN(c4ccc5c(c4)-c4nc(N6C(=O)OC[C@H]6C(F)F)cn4CCO5)CC3)C2)cc1] | 688 |
| 138 | 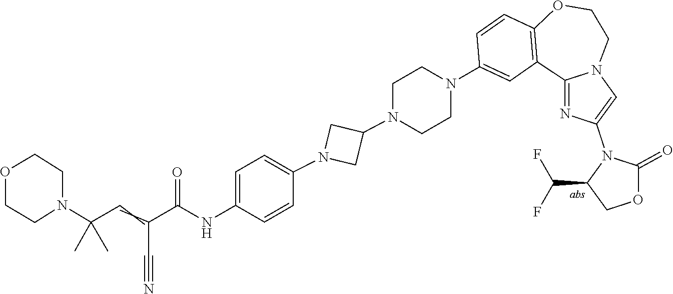 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)N1CCOCC1] | 758 |
| 139 | 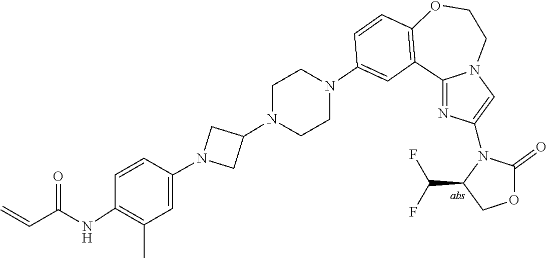 [SMILES: C=CC(=O)Nc1ccc(N2CC(N3CCN(c4ccc5c(c4)-c4nc(N6C(=O)OC[C@H]6C(F)F)cn4CCO5)CC3)C2)cc1C] | 620 |
| 140 | 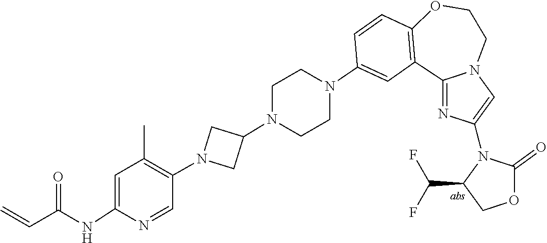 [SMILES: C=CC(=O)Nc1cc(C)c(N2CC(N3CCN(c4ccc5c(c4)-c4nc(N6C(=O)OC[C@H]6C(F)F)cn4CCO5)CC3)C2)cn1] | 621 |
| 141 | 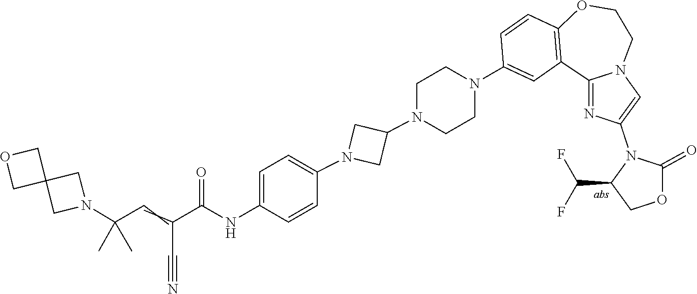 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)N1CC2(COC2)C1] | 770 |
| 142 | 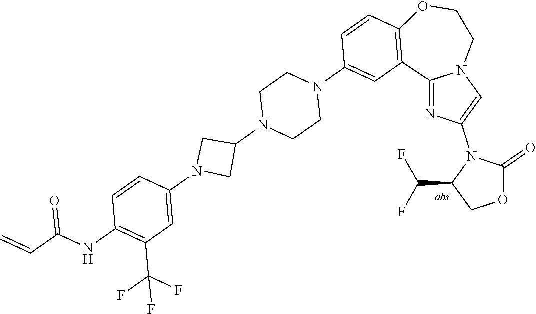 [SMILES: C=CC(=O)Nc1ccc(N2CC(N3CCN(c4ccc5c(c4)-c4nc(N6C(=O)OC[C@H]6C(F)F)cn4CCO5)CC3)C2)cc1C(F)(F)F] | 674 |
| 143 | 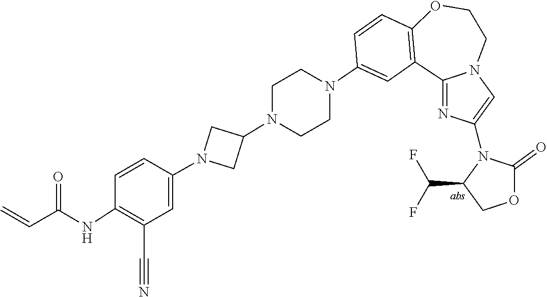 [SMILES: C=CC(=O)Nc1ccc(N2CC(N3CCN(c4ccc5c(c4)-c4nc(N6C(=O)OC[C@H]6C(F)F)cn4CCO5)CC3)C2)cc1C#N] | 631 |
| 144 | 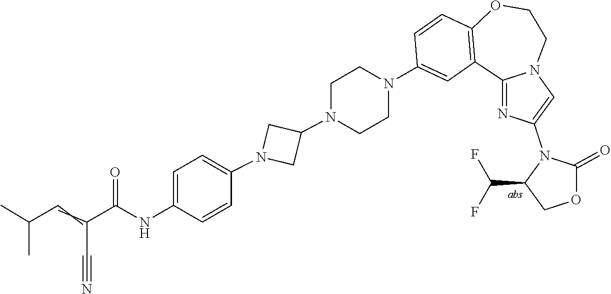 [SMILES: CC(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1] | 673 |
| 145 | 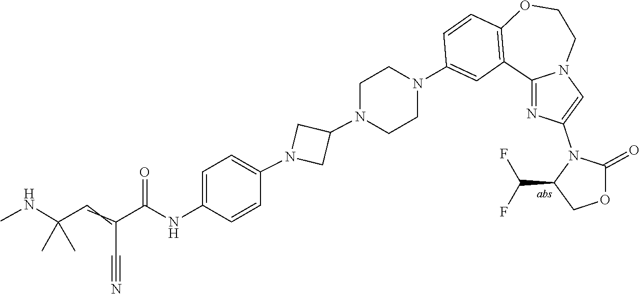 [SMILES: CNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1] | 702 |
| 146 | 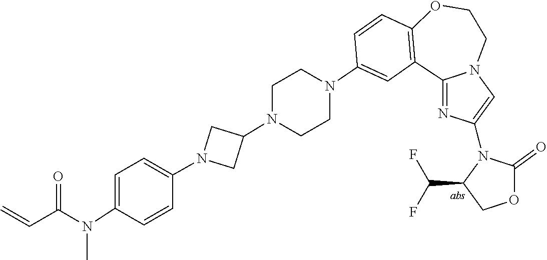 [SMILES: C=CC(=O)N(C)c1ccc(N2CC(N3CCN(c4ccc5c(c4)-c4nc(N6C(=O)OC[C@H]6C(F)F)cn4CCO5)CC3)C2)cc1] | 620 |
| 147 | 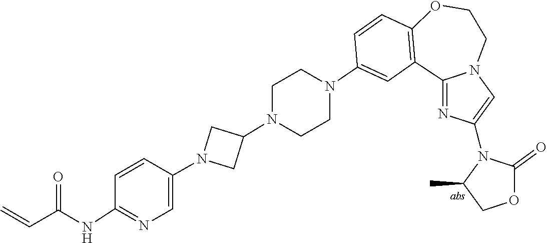 [SMILES: C=CC(=O)Nc1ccc(N2CC(N3CCN(c4ccc5c(c4)-c4nc(N6C(=O)OC[C@H]6C)cn4CCO5)CC3)C2)cn1] | 571 |
| 148 | 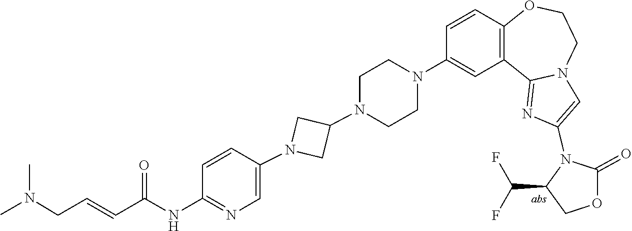 [SMILES: CN(C)C/C=C/C(=O)Nc1ccc(N2CC(N3CCN(c4ccc5c(c4)-c4nc(N6C(=O)OC[C@H]6C(F)F)cn4CCO5)CC3)C2)cn1] | 664 |
| 149 | 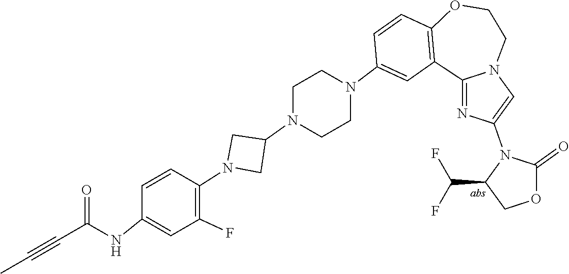 [SMILES: CC#CC(=O)Nc1ccc(N2CC(N3CCN(c4ccc5c(c4)-c4nc(N6C(=O)OC[C@H]6C(F)F)cn4CCO5)CC3)C2)c(F)c1] | 636 |
| 150 | 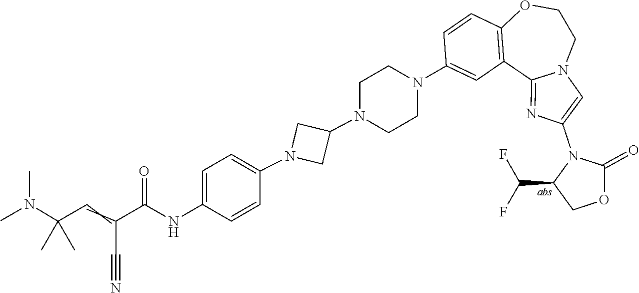 [SMILES: CN(C)C(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1] | 716 |
| 151 | 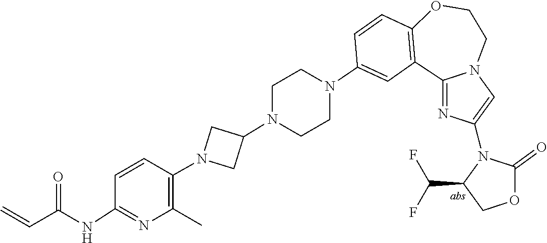 [SMILES: C=CC(=O)Nc1ccc(N2CC(N3CCN(c4ccc5c(c4)-c4nc(N6C(=O)OC[C@H]6C(F)F)cn4CCO5)CC3)C2)c(C)n1] | 621 |
| 152 | 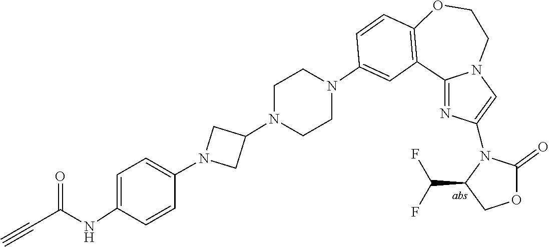 [SMILES: C#CC(=O)Nc1ccc(N2CC(N3CCN(c4ccc5c(c4)-c4nc(N6C(=O)OC[C@H]6C(F)F)cn4CCO5)CC3)C2)cc1] | 604 |
| 153 | 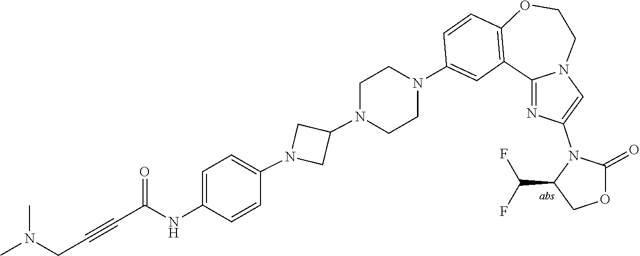 [SMILES: CN(C)CC#CC(=O)Nc1ccc(N2CC(N3CCN(c4ccc5c(c4)-c4nc(N6C(=O)OC[C@H]6C(F)F)cn4CCO5)CC3)C2)cc1] | 661 |
| 154 | 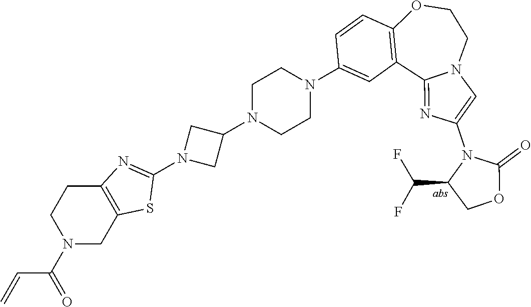 [SMILES: C=CC(=O)N1CCc2nc(N3CC(N4CCN(c5ccc6c(c5)-c5nc(N7C(=O)OC[C@H]7C(F)F)cn5CCO6)CC4)C3)sc2C1] | 653 |
| 155 | 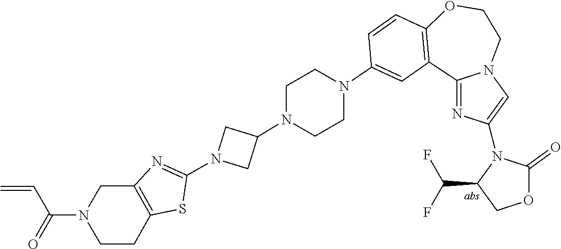 [SMILES: C=CC(=O)N1CCc2sc(N3CC(N4CCN(c5ccc6c(c5)-c5nc(N7C(=O)OC[C@H]7C(F)F)cn5CCO6)CC4)C3)nc2C1] | 653 |
| 156 | 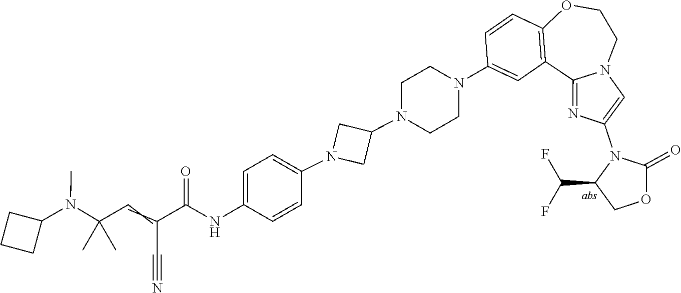 [SMILES: CN(C1CCC1)C(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1] | 756 |
| 157 | 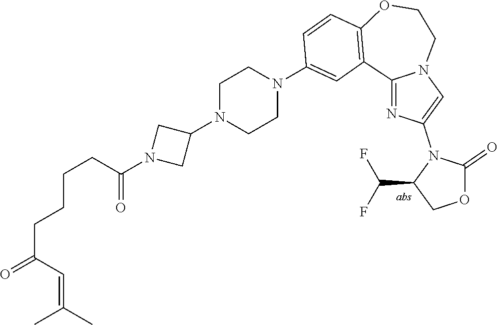 [SMILES: CC(C)=CC(=O)CCCCC(=O)N1CC(N2CCN(c3ccc4c(c3)-c3nc(N5C(=O)OC[C@H]5C(F)F)cn3CCO4)CC2)C1] | 627 |
| 158 | 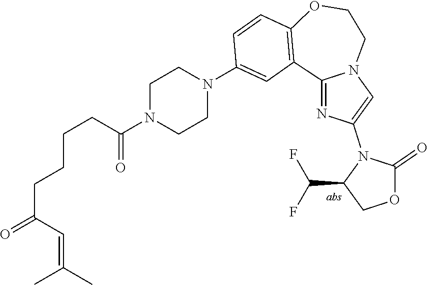 [SMILES: CC(C)=CC(=O)CCCCC(=O)N1CCN(c2ccc3c(c2)-c2nc(N4C(=O)OC[C@H]4C(F)F)cn2CCO3)CC1] | 572 |
| 159 | 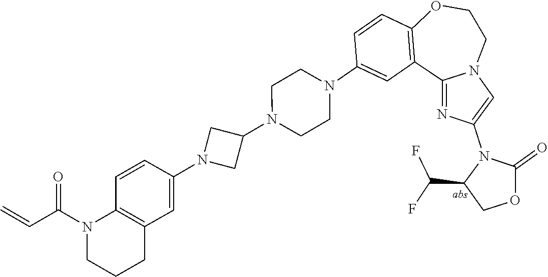 [SMILES: C=CC(=O)N1CCCc2cc(N3CC(N4CCN(c5ccc6c(c5)-c5nc(N7C(=O)OC[C@H]7C(F)F)cn5CCO6)CC4)C3)ccc21] | 646 |
| 160 | 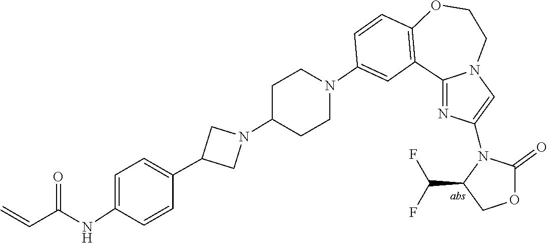 [SMILES: C=CC(=O)Nc1ccc(C2CN(C3CCN(c4ccc5c(c4)-c4nc(N6C(=O)OC[C@H]6C(F)F)cn4CCO5)CC3)C2)cc1] | 605 |
| 161 | 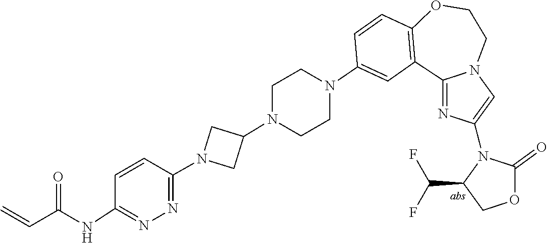 [SMILES: C=CC(=O)Nc1ccc(N2CC(N3CCN(c4ccc5c(c4)-c4nc(N6C(=O)OC[C@H]6C(F)F)cn4CCO5)CC3)C2)nn1] | 608 |
| 162 | 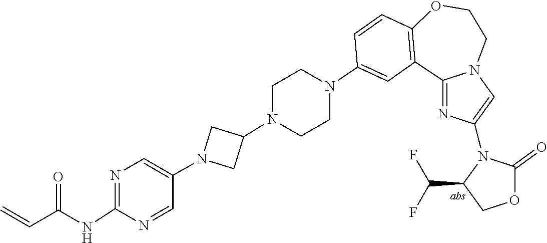 [SMILES: C=CC(=O)Nc1ncc(N2CC(N3CCN(c4ccc5c(c4)-c4nc(N6C(=O)OC[C@H]6C(F)F)cn4CCO5)CC3)C2)cn1] | 608 |
| 163 | 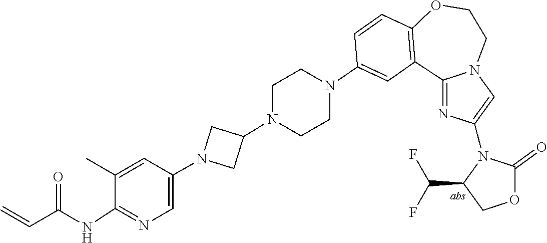 [SMILES: C=CC(=O)Nc1ncc(N2CC(N3CCN(c4ccc5c(c4)-c4nc(N6C(=O)OC[C@H]6C(F)F)cn4CCO5)CC3)C2)cc1C] | 621 |
| 164 | 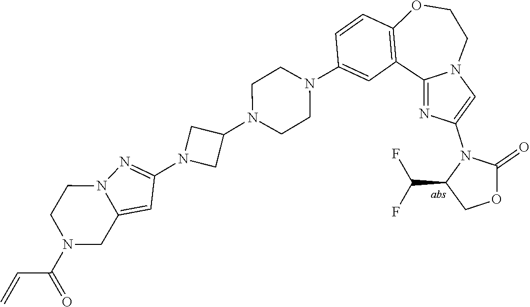 [SMILES: C=CC(=O)N1CCn2nc(N3CC(N4CCN(c5ccc6c(c5)-c5nc(N7C(=O)OC[C@H]7C(F)F)cn5CCO6)CC4)C3)cc2C1] | 636 |
| 165 | 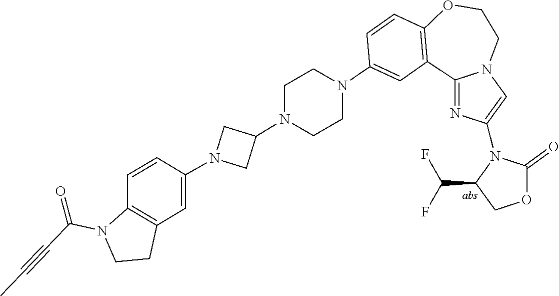 [SMILES: CC#CC(=O)N1CCc2cc(N3CC(N4CCN(c5ccc6c(c5)-c5nc(N7C(=O)OC[C@H]7C(F)F)cn5CCO6)CC4)C3)ccc21] | 644 |
| 166 | 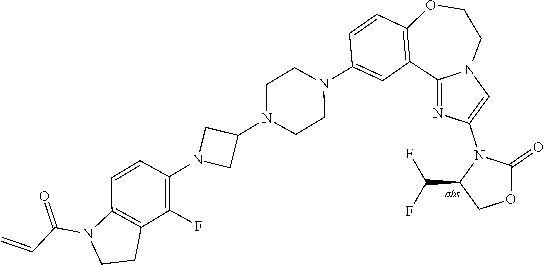 [SMILES: C=CC(=O)N1CCc2c1ccc(N1CC(N3CCN(c4ccc5c(c4)-c4nc(N6C(=O)OC[C@H]6C(F)F)cn4CCO5)CC3)C1)c2F] | 650 |
| 167 | 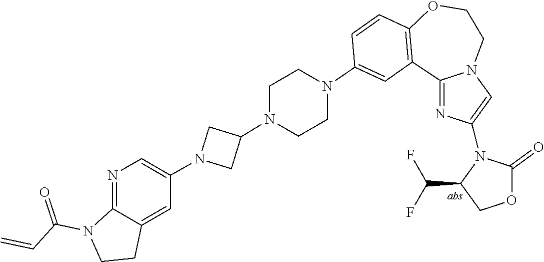 [SMILES: C=CC(=O)N1CCc2cc(N3CC(N4CCN(c5ccc6c(c5)-c5nc(N7C(=O)OC[C@H]7C(F)F)cn5CCO6)CC4)C3)cnc21] | 633 |
| 168 | 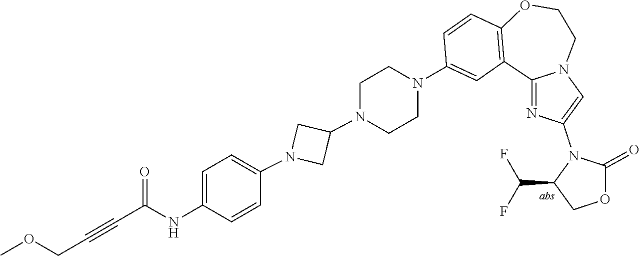 [SMILES: COCC#CC(=O)Nc1ccc(N2CC(N3CCN(c4ccc5c(c4)-c4nc(N6C(=O)OC[C@H]6C(F)F)cn4CCO5)CC3)C2)cc1] | 648 |
| 169 | 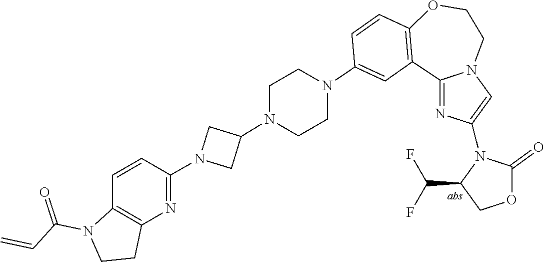 [SMILES: C=CC(=O)N1CCc2nc(N3CC(N4CCN(c5ccc6c(c5)-c5nc(N7C(=O)OC[C@H]7C(F)F)cn5CCO6)CC4)C3)ccc21] | 633 |
| 170 | 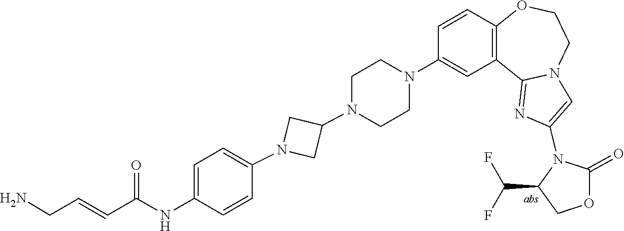 [SMILES: NC/C=C/C(=O)Nc1ccc(N2CC(N3CCN(c4ccc5c(c4)-c4nc(N6C(=O)OC[C@H]6C(F)F)cn4CCO5)CC3)C2)cc1] | 635 |
| 171 | 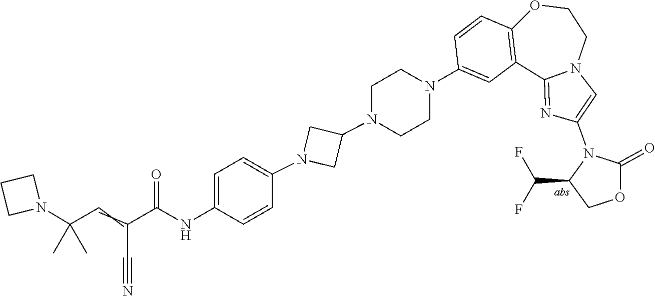 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)N1CCC1] | 728 |
| 172 | 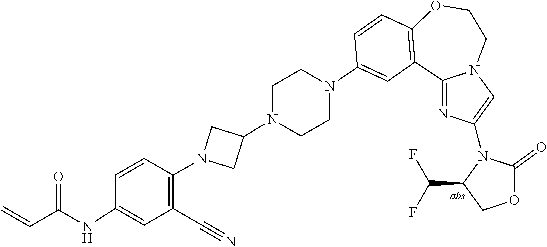 [SMILES: C=CC(=O)Nc1ccc(N2CC(N3CCN(c4ccc5c(c4)-c4nc(N6C(=O)OC[C@H]6C(F)F)cn4CCO5)CC3)C2)c(C#N)c1] | 631 |
| 173 | 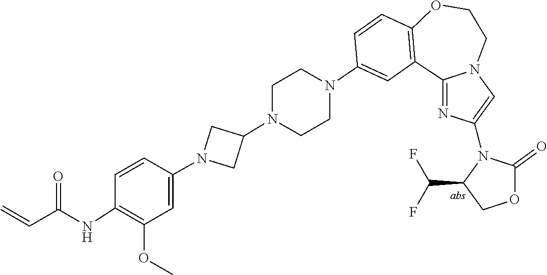 [SMILES: C=CC(=O)Nc1ccc(N2CC(N3CCN(c4ccc5c(c4)-c4nc(N6C(=O)OC[C@H]6C(F)F)cn4CCO5)CC3)C2)cc1OC] | 636 |
| 174 | 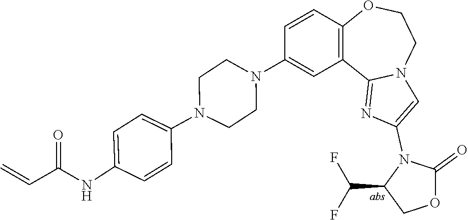 [SMILES: C=CC(=O)Nc1ccc(N2CCN(c3ccc4c(c3)-c3nc(N5C(=O)OC[C@H]5C(F)F)cn3CCO4)CC2)cc1] | 551 |
| 175 | 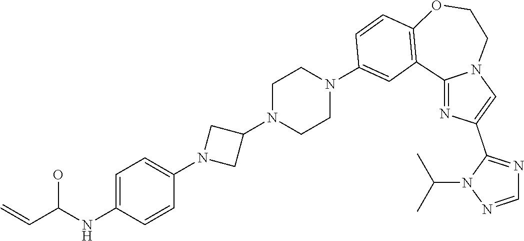 [SMILES: C=CC(O)Nc1ccc(N2CC(N3CCN(c4ccc5c(c4)-c4nc(-c6ncnn6C(C)C)cn4CCO5)CC3)C2)cc1] | 580 |
| 176 | 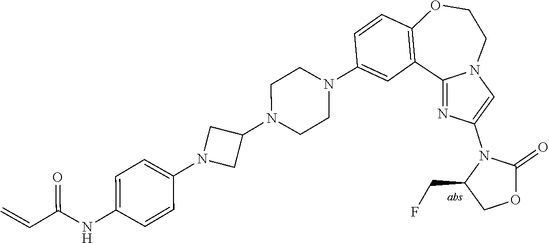 [SMILES: C=CC(=O)Nc1ccc(N2CC(N3CCN(c4ccc5c(c4)-c4nc(N6C(=O)OC[C@H]6CF)cn4CCO5)CC3)C2)cc1] | 588 |
| 177 | 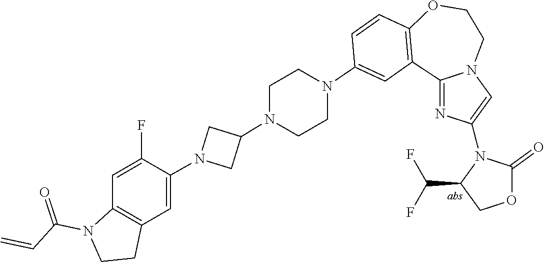 [SMILES: C=CC(=O)N1CCc2cc(N3CC(N4CCN(c5ccc6c(c5)-c5nc(N7C(=O)OC[C@H]7C(F)F)cn5CCO6)CC4)C3)c(F)cc21] | 650 |
| 178 | 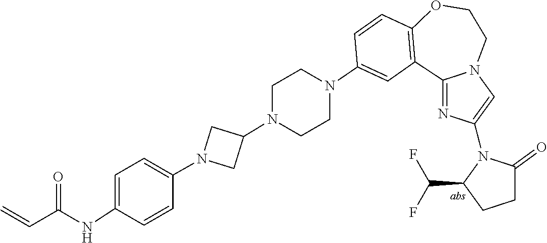 [SMILES: C=CC(=O)Nc1ccc(N2CC(N3CCN(c4ccc5c(c4)-c4nc(N6C(=O)CC[C@H]6C(F)F)cn4CCO5)CC3)C2)cc1] | 604 |
| 179 | 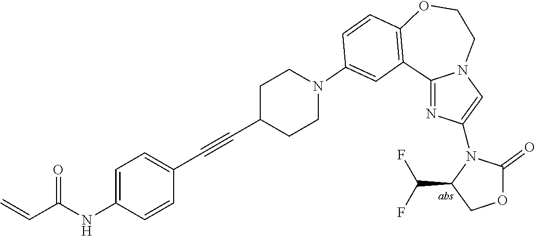 [SMILES: C=CC(=O)Nc1ccc(C#CC2CCN(c3ccc4c(c3)-c3nc(N5C(=O)OC[C@H]5C(F)F)cn3CCO4)CC2)cc1] | 574 |
| 180 | 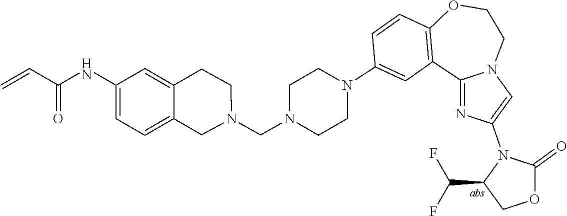 [SMILES: C=CC(=O)Nc1ccc2c(c1)CCN(CN1CCN(c3ccc4c(c3)-c3nc(N5C(=O)OC[C@H]5C(F)F)cn3CCO4)CC1)C2] | 619 |
| 181 | 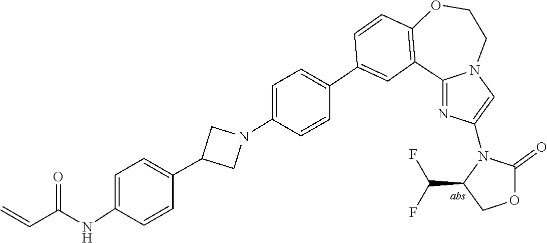 [SMILES: C=CC(=O)Nc1ccc(C2CN(c3ccc(-c4ccc5c(c4)-c4nc(N6C(=O)OC[C@H]6C(F)F)cn4CCO5)cc3)C2)cc1] | 598 |
| 182 | 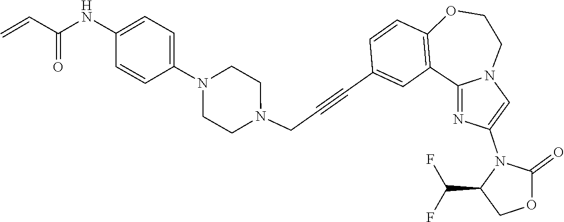 [SMILES: C=CC(=O)Nc1ccc(N2CCN(CC#Cc3ccc4c(c3)-c3nc(N5C(=O)OC[C@H]5C(F)F)cn3CCO4)CC2)cc1] | 589 |
| 183 | 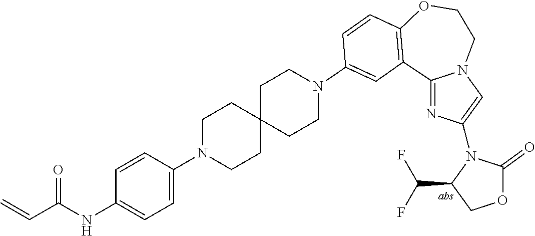 [SMILES: C=CC(=O)Nc1ccc(N2CCC3(CC2)CCN(c2ccc4c(c2)-c2nc(N5C(=O)OC[C@H]5C(F)F)cn2CCO4)CC3)cc1] | 619 |
| 184 | 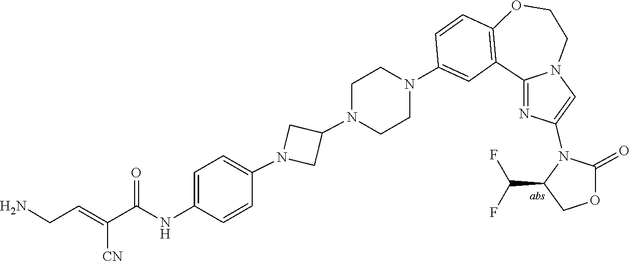 [SMILES: N#C/C(=C\CN)C(=O)Nc1ccc(N2CC(N3CCN(c4ccc5c(c4)-c4nc(N6C(=O)OC[C@H]6C(F)F)cn4CCO5)CC3)C2)cc1] | 660 |
| 185 | 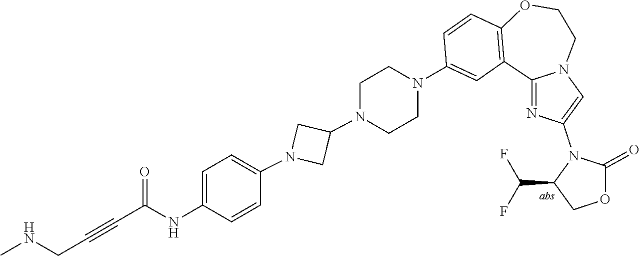 [SMILES: CNCC#CC(=O)Nc1ccc(N2CC(N3CCN(c4ccc5c(c4)-c4nc(N6C(=O)OC[C@H]6C(F)F)cn4CCO5)CC3)C2)cc1] | 647 |
| 186 | 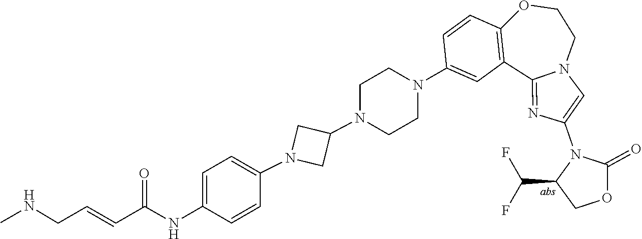 [SMILES: CNC/C=C/C(=O)Nc1ccc(N2CC(N3CCN(c4ccc5c(c4)-c4nc(N6C(=O)OC[C@H]6C(F)F)cn4CCO5)CC3)C2)cc1] | 649 |
| 187 | 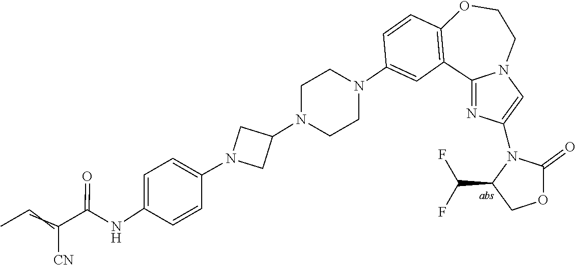 [SMILES: CC=C(C#N)C(=O)Nc1ccc(N2CC(N3CCN(c4ccc5c(c4)-c4nc(N6C(=O)OC[C@H]6C(F)F)cn4CCO5)CC3)C2)cc1] | 645 |
| 188 | 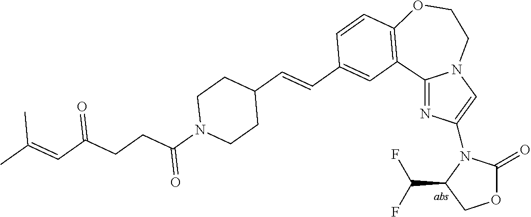 [SMILES: CC(C)=CC(=O)CCC(=O)N1CCC(/C=C/c2ccc3c(c2)-c2nc(N4C(=O)OC[C@H]4C(F)F)cn2CCO3)CC1] | 669 |
| 189 | 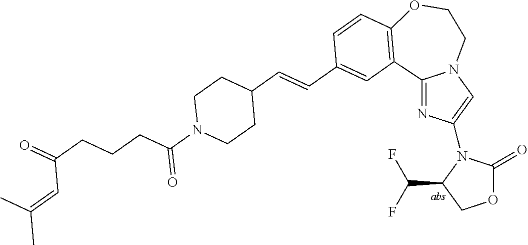 [SMILES: CC(C)=CC(=O)CCCC(=O)N1CCC(/C=C/c2ccc3c(c2)-c2nc(N4C(=O)OC[C@H]4C(F)F)cn2CCO3)CC1] | 583 |
| 190 | 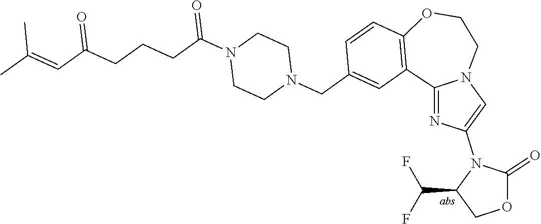 [SMILES: CC(C)=CC(=O)CCCC(=O)N1CCN(Cc2ccc3c(c2)-c2nc(N4C(=O)OC[C@H]4C(F)F)cn2CCO3)CC1] | 572 |
| 191 | 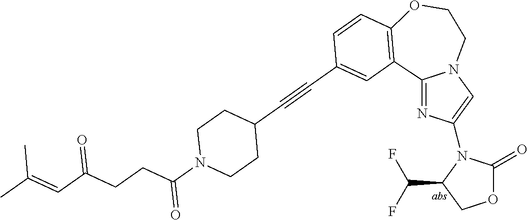 [SMILES: CC(C)=CC(=O)CCC(=O)N1CCC(C#Cc2ccc3c(c2)-c2nc(N4C(=O)OC[C@H]4C(F)F)cn2CCO3)CC1] | 567 |
| 192 | 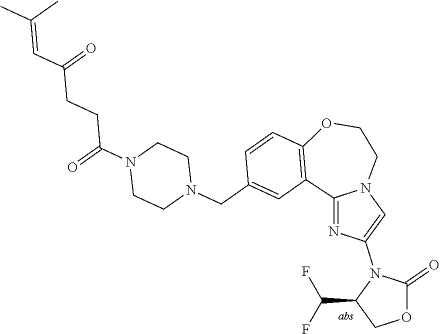 [SMILES: CC(C)=CC(=O)CCC(=O)N1CCN(Cc2ccc3c(c2)-c2nc(N4C(=O)OC[C@H]4C(F)F)cn2CCO3)CC1] | 558 |
| 193 | 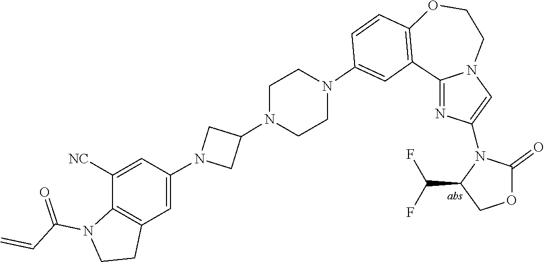 [SMILES: C=CC(=O)N1CCc2cc(N3CC(N4CCN(c5ccc6c(c5)-c5nc(N7C(=O)OC[C@H]7C(F)F)cn5CCO6)CC4)C3)cc(C#N)c21] | 657 |
| 194 | 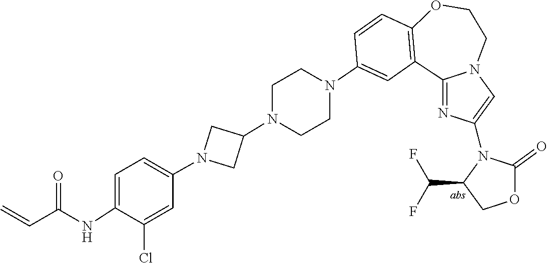 [SMILES: C=CC(=O)Nc1ccc(N2CC(N3CCN(c4ccc5c(c4)-c4nc(N6C(=O)OC[C@H]6C(F)F)cn4CCO5)CC3)C2)cc1Cl] | 640 |
| 195 | 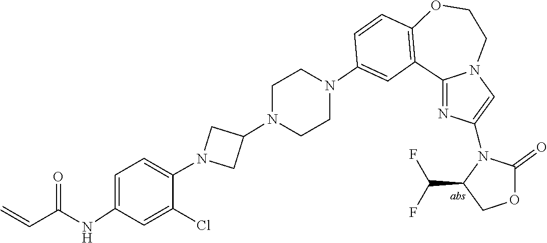 [SMILES: C=CC(=O)Nc1ccc(N2CC(N3CCN(c4ccc5c(c4)-c4nc(N6C(=O)OC[C@H]6C(F)F)cn4CCO5)CC3)C2)c(Cl)c1] | 640 |
| 196 | 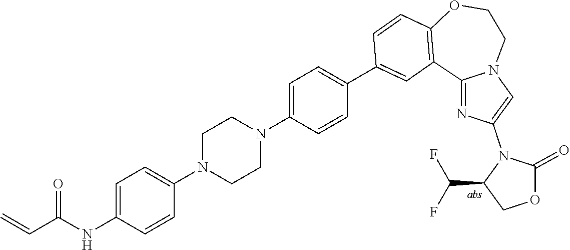 [SMILES: C=CC(=O)Nc1ccc(N2CCN(c3ccc(-c4ccc5c(c4)-c4nc(N6C(=O)OC[C@H]6C(F)F)cn4CCO5)cc3)CC2)cc1] | 627 |
| 197 | 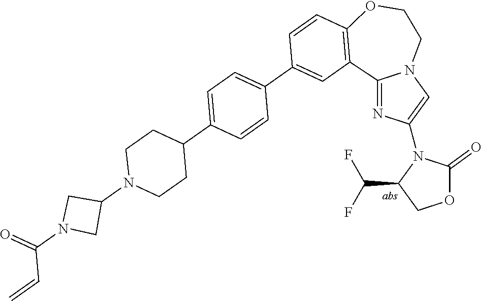 [SMILES: C=CC(=O)N1CC(N2CCC(c3ccc(-c4ccc5c(c4)-c4nc(N6C(=O)OC[C@H]6C(F)F)cn4CCO5)cc3)CC2)C1] | 590 |
| 198 | 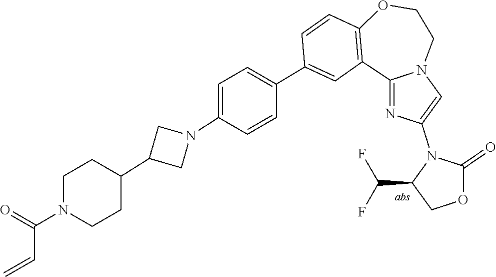 [SMILES: C=CC(=O)N1CCC(C2CN(c3ccc(-c4ccc5c(c4)-c4nc(N6C(=O)OC[C@H]6C(F)F)cn4CCO5)cc3)C2)CC1] | 590 |
| 199 | 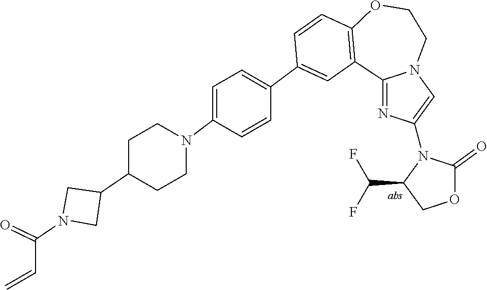 [SMILES: C=CC(=O)N1CC(C2CCN(c3ccc(-c4ccc5c(c4)-c4nc(N6C(=O)OC[C@H]6C(F)F)cn4CCO5)cc3)CC2)C1] | 590 |
| 200 | 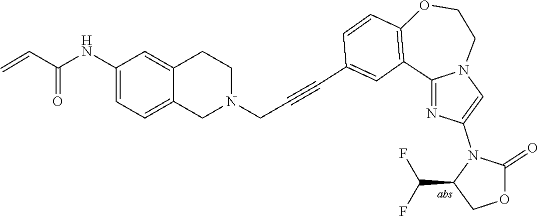 [SMILES: C=CC(=O)Nc1ccc2c(c1)CCN(CC#Cc1ccc3c(c1)-c1nc(N4C(=O)OC[C@H]4C(F)F)cn1CCO3)C2] | 560 |
| 201 | 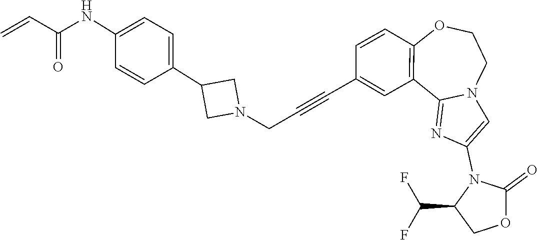 [SMILES: C=CC(=O)Nc1ccc(C2CN(CC#Cc3ccc4c(c3)-c3nc(N5C(=O)OC[C@H]5C(F)F)cn3CCO4)C2)cc1] | 560 |
| 202 | 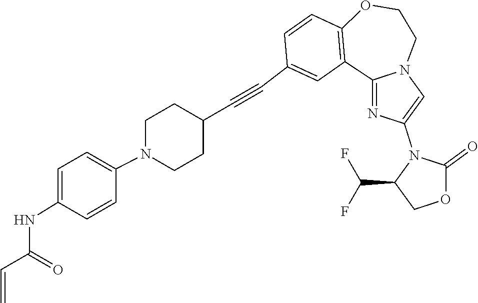 [SMILES: C=CC(=O)Nc1ccc(N2CCC(C#Cc3ccc4c(c3)-c3nc(N5C(=O)OC[C@H]5C(F)F)cn3CCO4)CC2)cc1] | 574 |
| 203 | 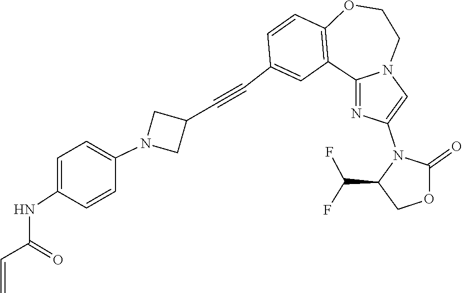 [SMILES: C=CC(=O)Nc1ccc(N2CC(C#Cc3ccc4c(c3)-c3nc(N5C(=O)OC[C@H]5C(F)F)cn3CCO4)C2)cc1] | 546 |
| 204 | 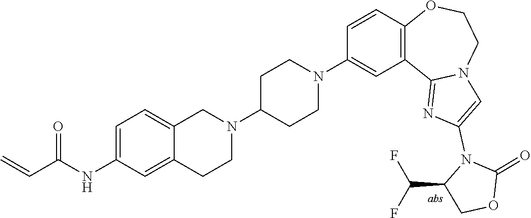 [SMILES: C=CC(=O)Nc1ccc2c(c1)CCN(C1CCN(c3ccc4c(c3)-c3nc(N5C(=O)OC[C@H]5C(F)F)cn3CCO4)CC1)C2] | 605 |
| 205 | 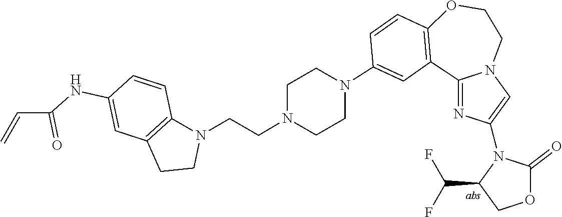 [SMILES: C=CC(=O)Nc1ccc2c(c1)CCN2CCN1CCN(c2ccc3c(c2)-c2nc(N4C(=O)OC[C@H]4C(F)F)cn2CCO3)CC1] | 620 |
| 206 | 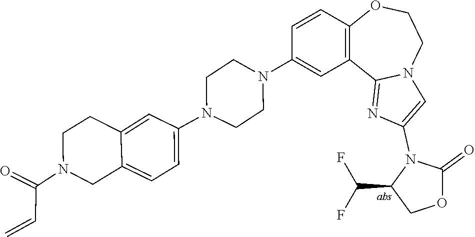 [SMILES: C=CC(=O)N1CCc2cc(N3CCN(c4ccc5c(c4)-c4nc(N6C(=O)OC[C@H]6C(F)F)cn4CCO5)CC3)ccc2C1] | 591 |
| 207 | 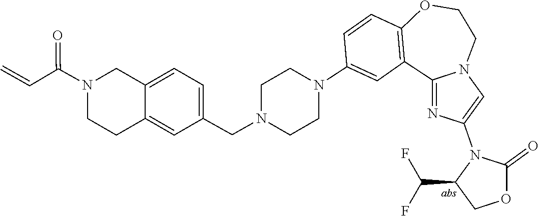 [SMILES: C=CC(=O)N1CCc2cc(CN3CCN(c4ccc5c(c4)-c4nc(N6C(=O)OC[C@H]6C(F)F)cn4CCO5)CC3)ccc2C1] | 605 |
| 208 | 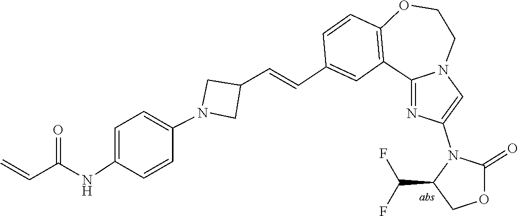 [SMILES: C=CC(=O)Nc1ccc(N2CC(/C=C/c3ccc4c(c3)-c3nc(N5C(=O)OC[C@H]5C(F)F)cn3CCO4)C2)cc1] | 548 |
| 209 | 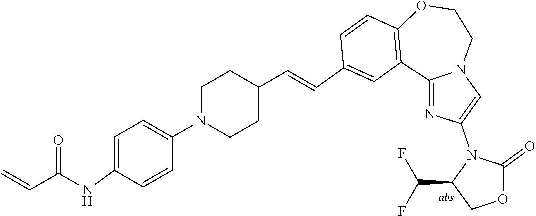 [SMILES: C=CC(=O)Nc1ccc(N2CCC(/C=C/c3ccc4c(c3)-c3nc(N5C(=O)OC[C@H]5C(F)F)cn3CCO4)CC2)cc1] | 576 |
| 210 | 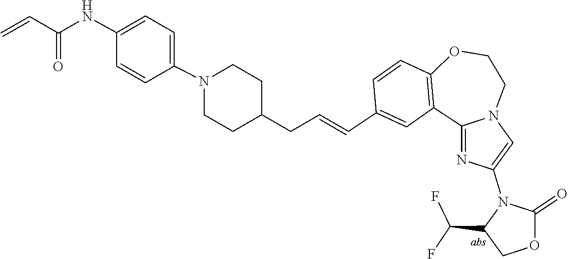 | 590 |
| 211 | 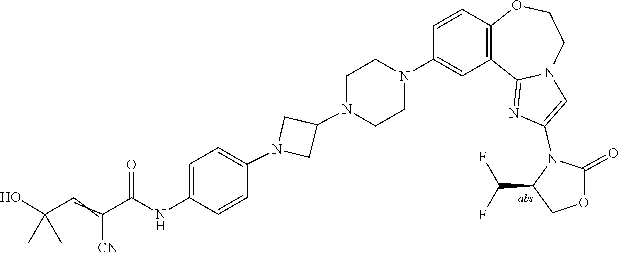 [SMILES: CC(C)(O)C=C(C#N)C(=O)Nc1ccc(N2CC(N3CCN(c4ccc5c(c4)-c4nc(N6C(=O)OC[C@H]6C(F)F)cn4CCO5)CC3)C2)cc1] | 689 |
| 212 | 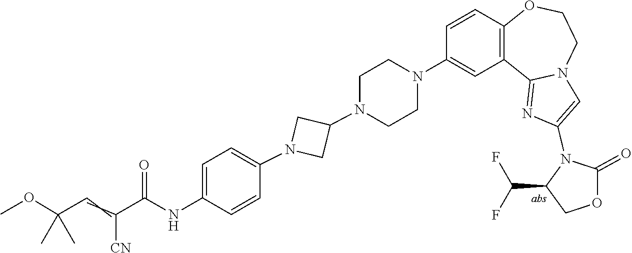 [SMILES: COC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1] | 703 |
| 213 | 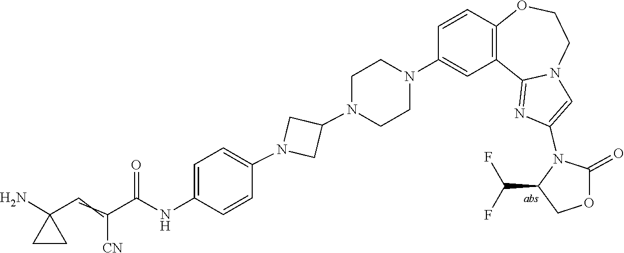 [SMILES: N#CC(=CC1(N)CC1)C(=O)Nc1ccc(N2CC(N3CCN(c4ccc5c(c4)-c4nc(N6C(=O)OC[C@H]6C(F)F)cn4CCO5)CC3)C2)cc1] | 686 |
| 214 | 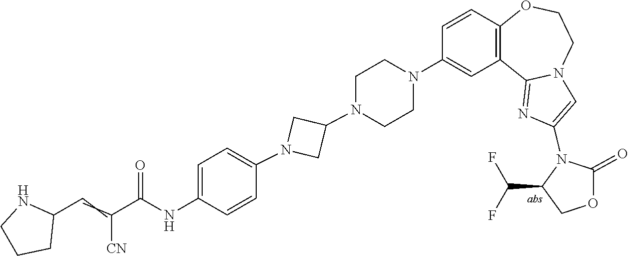 [SMILES: N#CC(=CC1CCCN1)C(=O)Nc1ccc(N2CC(N3CCN(c4ccc5c(c4)-c4nc(N6C(=O)OC[C@H]6C(F)F)cn4CCO5)CC3)C2)cc1] | 700 |
| 215 | 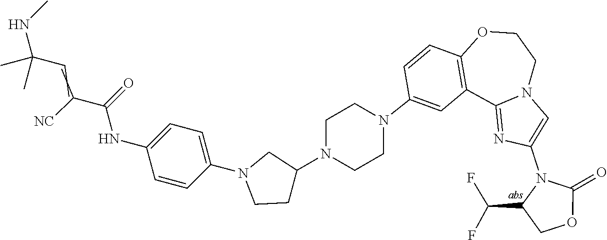 | 716 |
| 216 | 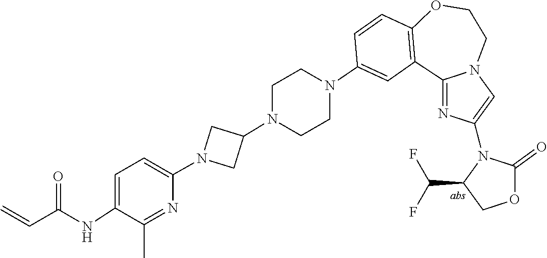 [SMILES: C=CC(=O)Nc1ccc(N2CC(N3CCN(c4ccc5c(c4)-c4nc(N6C(=O)OC[C@H]6C(F)F)cn4CCO5)CC3)C2)nc1C] | 621 |
| 217 | 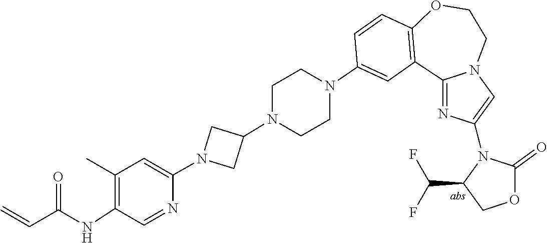 [SMILES: C=CC(=O)Nc1cnc(N2CC(N3CCN(c4ccc5c(c4)-c4nc(N6C(=O)OC[C@H]6C(F)F)cn4CCO5)CC3)C2)cc1C] | 621 |
| 218 | 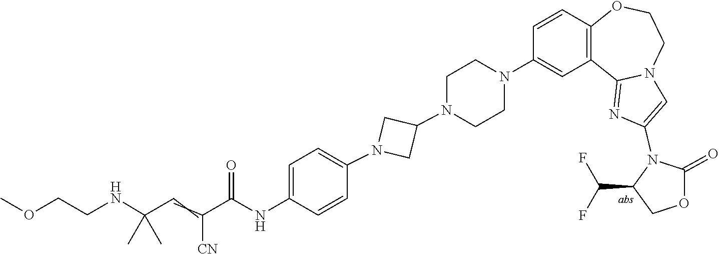 [SMILES: COCCNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1] | 746 |
| 219 | 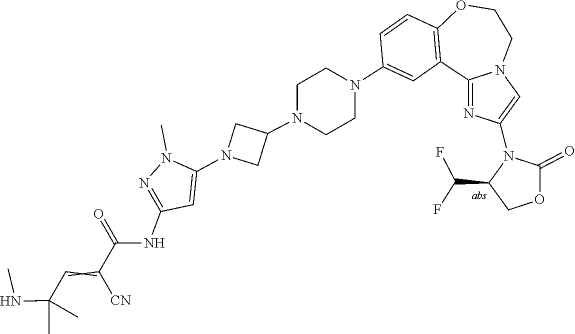 [SMILES: CNC(C)(C)C=C(C#N)C(=O)Nc1cc(N2CC(N3CCN(c4ccc5c(c4)-c4nc(N6C(=O)OC[C@H]6C(F)F)cn4CCO5)CC3)C2)n(C)n1] | 706 |
| 220 | 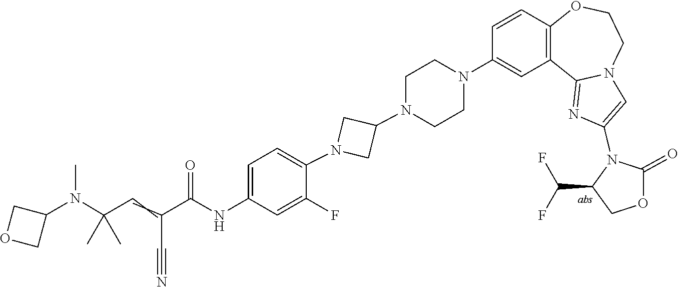 [SMILES: CN(C1COC1)C(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1] | 776 |
| 221 | 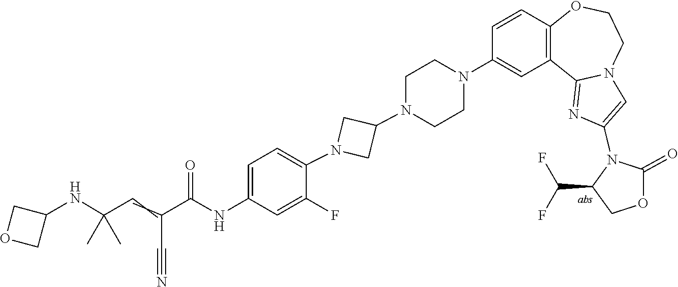 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1)NC1COC1] | 762 |
| 222 | 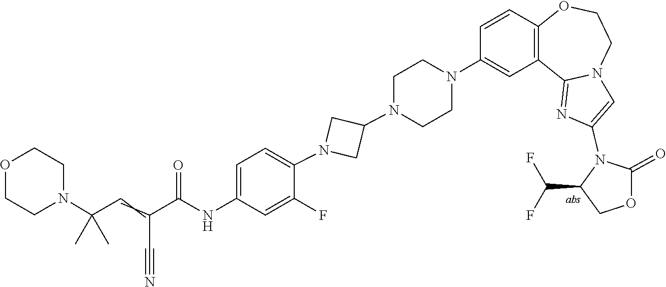 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1)N1CCOCC1] | 776 |
| 223 | 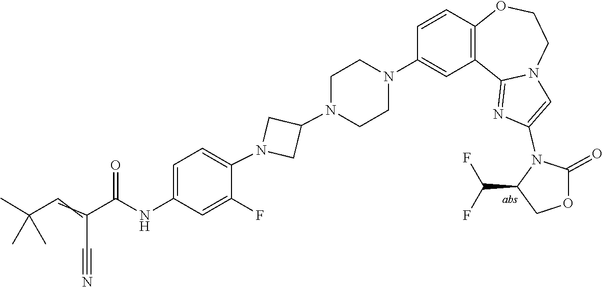 [SMILES: CC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1] | 705 |
| 224 | 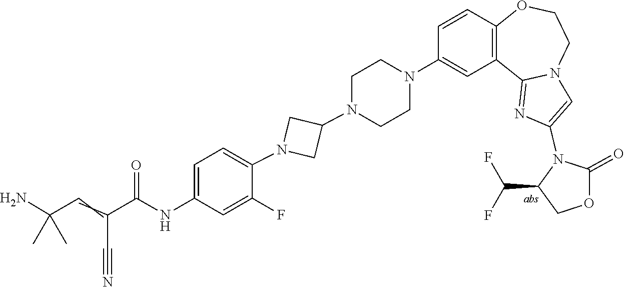 [SMILES: CC(C)(N)C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1] | 706 |
| 225 | 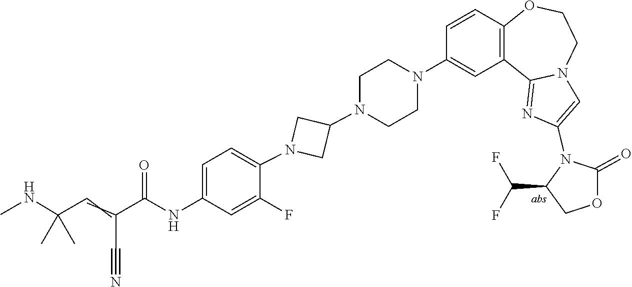 [SMILES: CNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1] | 720 |
| 226 | 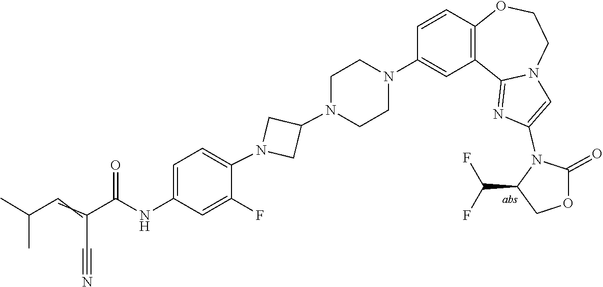 [SMILES: CC(C)C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1] | 691 |
| 227 | 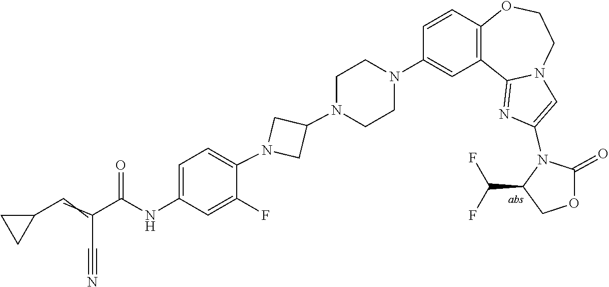 [SMILES: N#CC(=CC1CC1)C(=O)Nc1ccc(N2CC(N3CCN(c4ccc5c(c4)-c4nc(N6C(=O)OC[C@H]6C(F)F)cn4CCO5)CC3)C2)c(F)c1] | 689 |
| 228 | 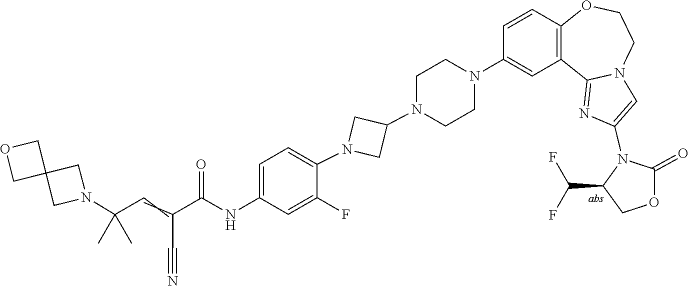 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1)N1CC2(COC2)C1] | 788 |
| 229 | 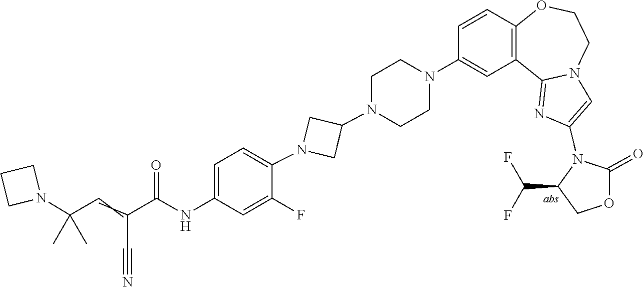 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1)N1CCC1] | 746 |
| 230 | 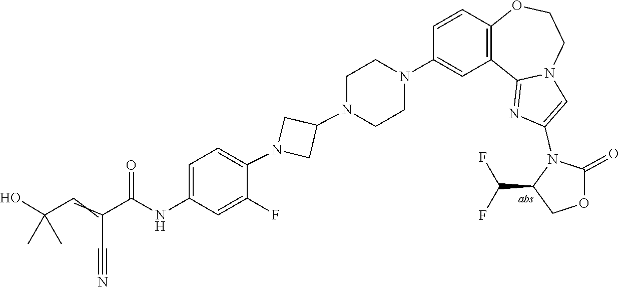 [SMILES: CC(C)(O)C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1] | 707 |
| 231 | 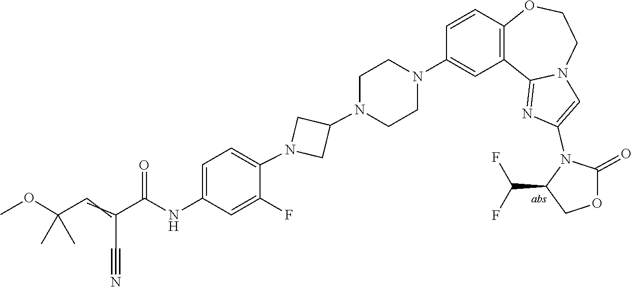 [SMILES: COC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1] | 721 |
| 232 | 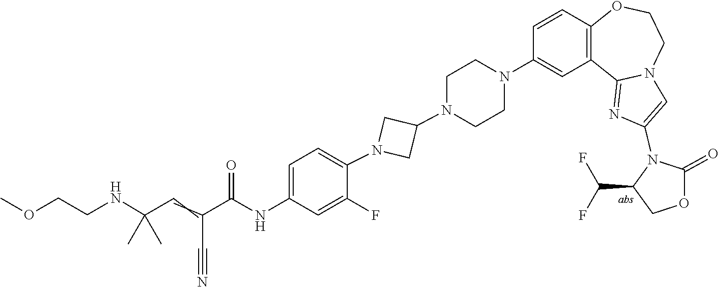 [SMILES: COCCNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1] | 764 |
| 233 | 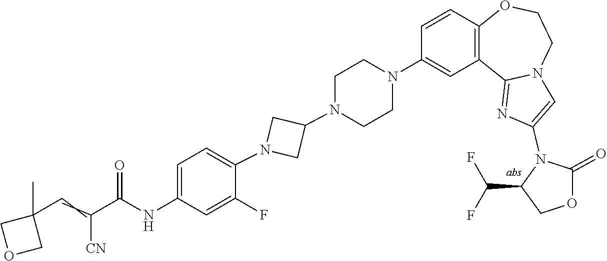 [SMILES: CC1(C=C(C#N)C(=O)Nc2ccc(N3CC(N4CCN(c5ccc6c(c5)-c5nc(N7C(=O)OC[C@H]7C(F)F)cn5CCO6)CC4)C3)c(F)c2)COC1] | 719 |
| 234 | 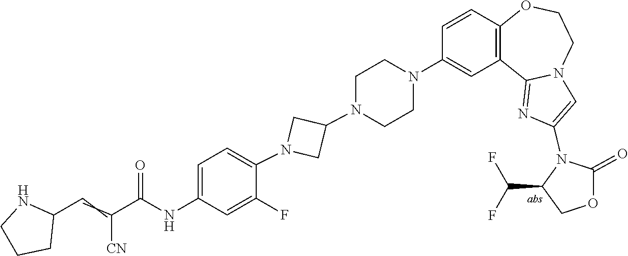 [SMILES: N#CC(=CC1CCCN1)C(=O)Nc1ccc(N2CC(N3CCN(c4ccc5c(c4)-c4nc(N6C(=O)OC[C@H]6C(F)F)cn4CCO5)CC3)C2)c(F)c1] | 718 |
| 235 | 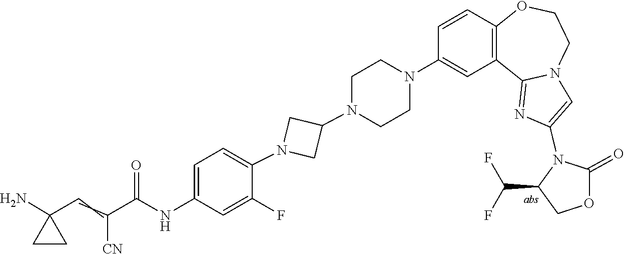 [SMILES: N#CC(=CC1(N)CC1)C(=O)Nc1ccc(N2CC(N3CCN(c4ccc5c(c4)-c4nc(N6C(=O)OC[C@H]6C(F)F)cn4CCO5)CC3)C2)c(F)c1] | 704 |
| 236 | 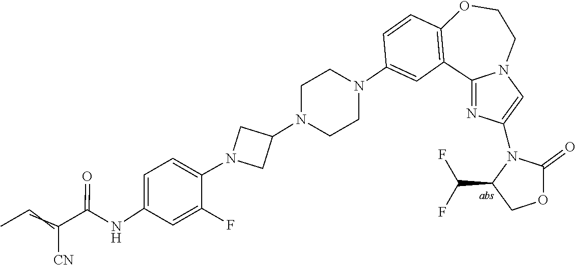 [SMILES: CC=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1] | 663 |
| 237 | 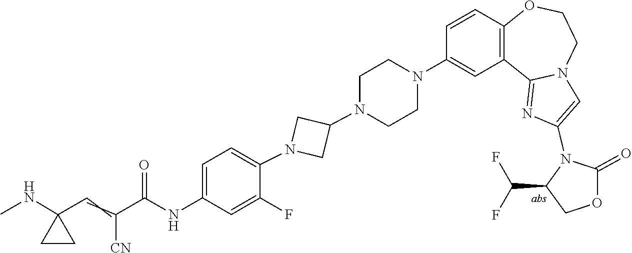 [SMILES: CNC1(C=C(C#N)C(=O)Nc2ccc(N3CC(N4CCN(c5ccc6c(c5)-c5nc(N7C(=O)OC[C@H]7C(F)F)cn5CCO6)CC4)C3)c(F)c2)CC1] | 718 |
| 238 | 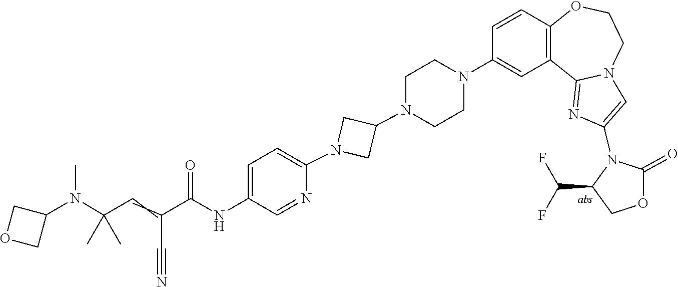 [SMILES: CN(C1COC1)C(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)nc1] | 759 |
| 239 | 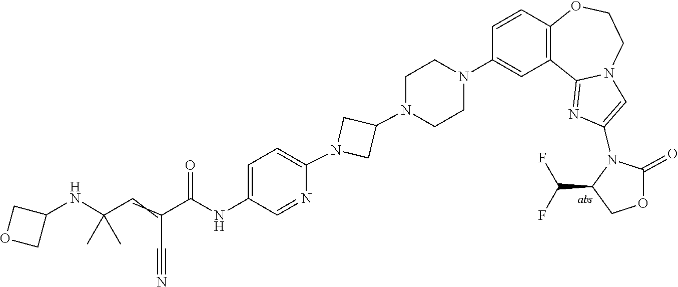 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)nc1)NC1COC1] | 745 |
| 240 | 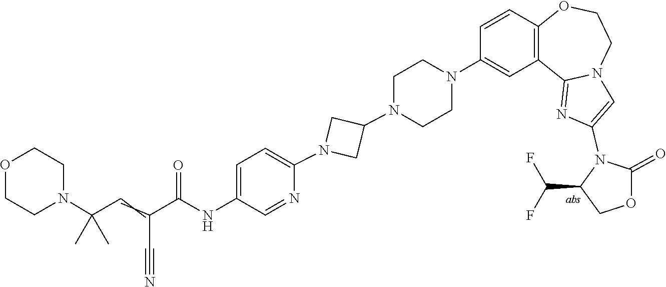 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)nc1)N1CCOCC1] | 759 |
| 241 | 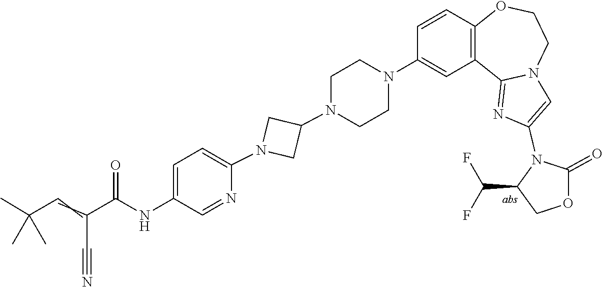 [SMILES: CC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)nc1] | 688 |
| 242 | 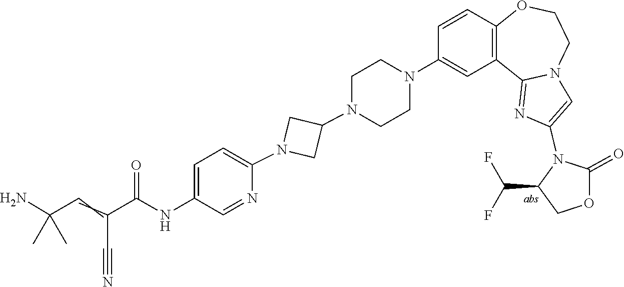 [SMILES: CC(C)(N)C=C(C#N)C(=O)Nc1ccc(N2CC(N3CCN(c4ccc5c(c4)-c4nc(N6C(=O)OC[C@H]6C(F)F)cn4CCO5)CC3)C2)nc1] | 689 |
| 243 | 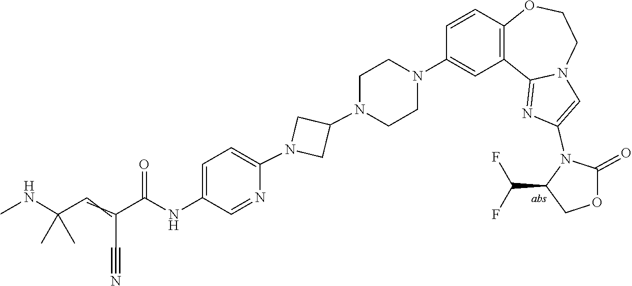 [SMILES: CNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)nc1] | 703 |
| 244 | 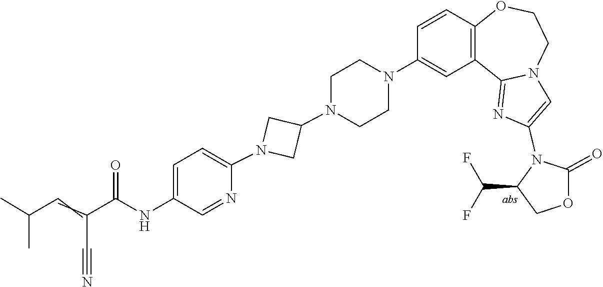 [SMILES: CC(C)C=C(C#N)C(=O)Nc1ccc(N2CC(N3CCN(c4ccc5c(c4)-c4nc(N6C(=O)OC[C@H]6C(F)F)cn4CCO5)CC3)C2)nc1] | 674 |
| 245 | 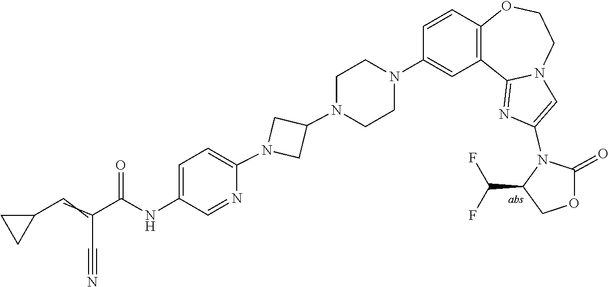 [SMILES: N#CC(=CC1CC1)C(=O)Nc1ccc(N2CC(N3CCN(c4ccc5c(c4)-c4nc(N6C(=O)OC[C@H]6C(F)F)cn4CCO5)CC3)C2)nc1] | 672 |
| 246 | 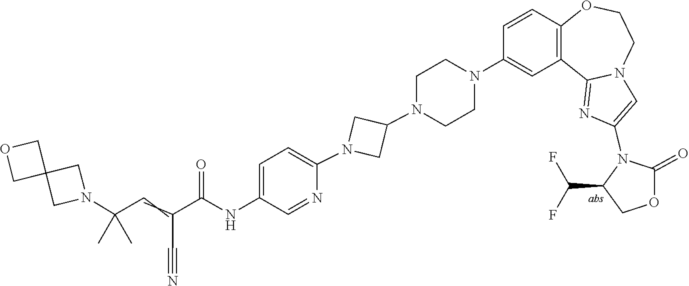 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)nc1)N1CC2(COC2)C1] | 771 |
| 247 | 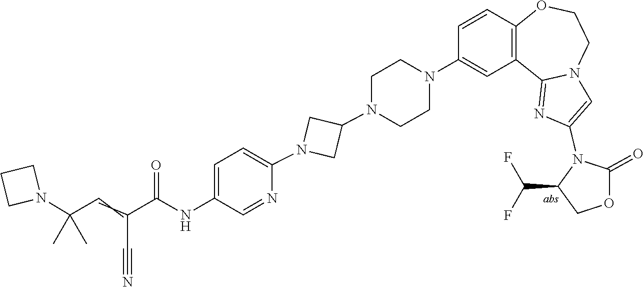 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)nc1)N1CCC1] | 729 |
| 248 | 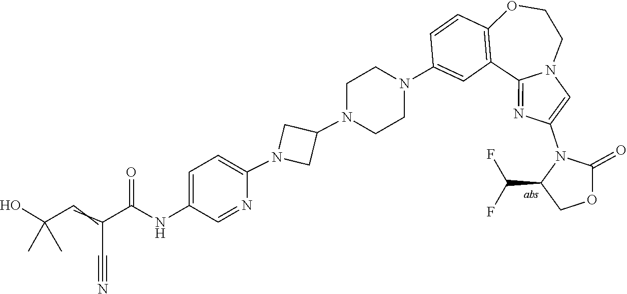 [SMILES: CC(C)(O)C=C(C#N)C(=O)Nc1ccc(N2CC(N3CCN(c4ccc5c(c4)-c4nc(N6C(=O)OC[C@H]6C(F)F)cn4CCO5)CC3)C2)nc1] | 690 |
| 249 | 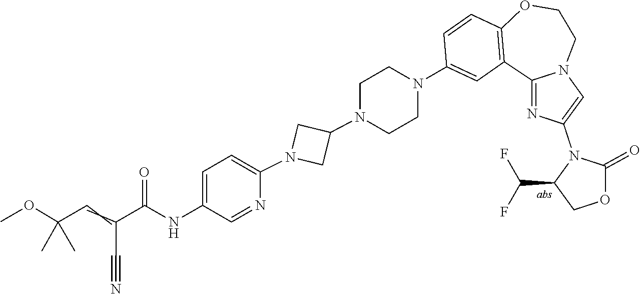 [SMILES: COC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)nc1] | 704 |
| 250 | 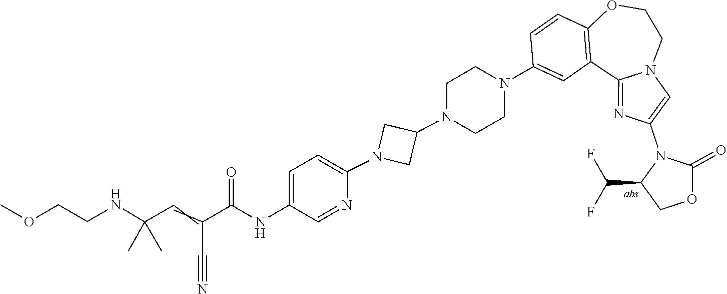 [SMILES: COCCNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)nc1] | 747 |
| 251 | 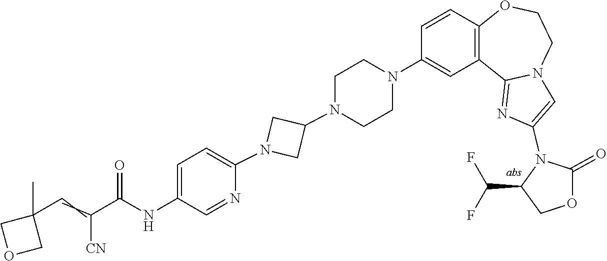 [SMILES: CC1(C=C(C#N)C(=O)Nc2ccc(N3CC(N4CCN(c5ccc6c(c5)-c5nc(N7C(=O)OC[C@H]7C(F)F)cn5CCO6)CC4)C3)nc2)COC1] | 702 |
| 252 | 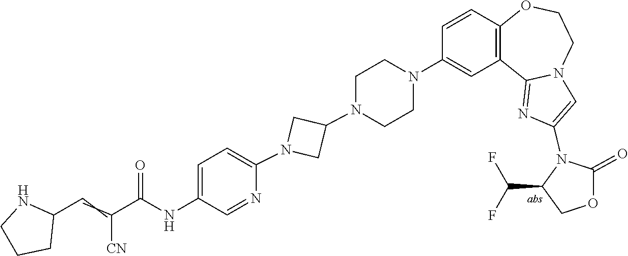 [SMILES: N#CC(=CC1CCCN1)C(=O)Nc1ccc(N2CC(N3CCN(c4ccc5c(c4)-c4nc(N6C(=O)OC[C@H]6C(F)F)cn4CCO5)CC3)C2)nc1] | 701 |
| 253 | 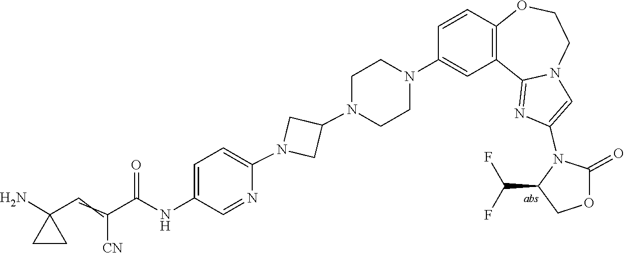 [SMILES: N#CC(=CC1(N)CC1)C(=O)Nc1ccc(N2CC(N3CCN(c4ccc5c(c4)-c4nc(N6C(=O)OC[C@H]6C(F)F)cn4CCO5)CC3)C2)nc1] | 687 |
| 254 | 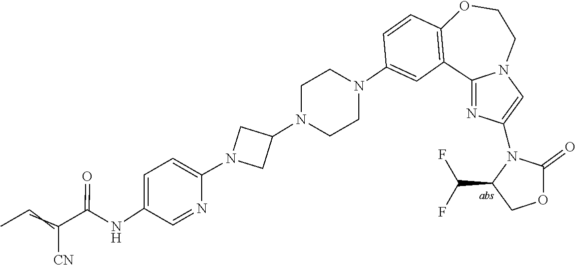 [SMILES: CC=C(C#N)C(=O)Nc1ccc(N2CC(N3CCN(c4ccc5c(c4)-c4nc(N6C(=O)OC[C@H]6C(F)F)cn4CCO5)CC3)C2)nc1] | 646 |
| 255 | 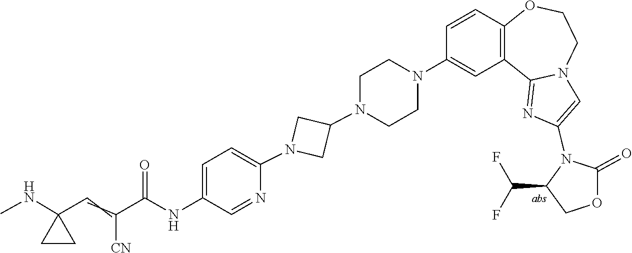 [SMILES: CNC1(C=C(C#N)C(=O)Nc2ccc(N3CC(N4CCN(c5ccc6c(c5)-c5nc(N7C(=O)OC[C@H]7C(F)F)cn5CCO6)CC4)C3)nc2)CC1] | 701 |
| 256 | 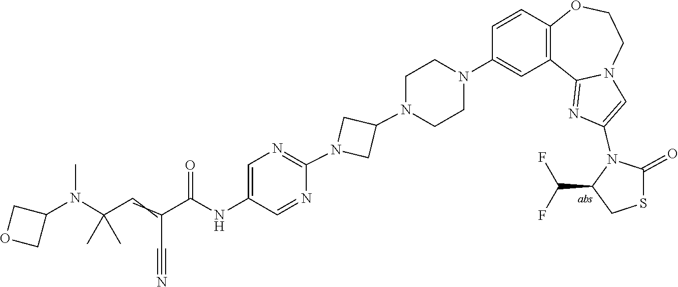 [SMILES: CN(C1COC1)C(C)(C)C=C(C#N)C(=O)Nc1cnc(N2CC(N3CCN(c4ccc5c(c4)-c4nc(N6C(=O)SC[C@H]6C(F)F)cn4CCO5)CC3)C2)nc1] | 760 |
| 257 | 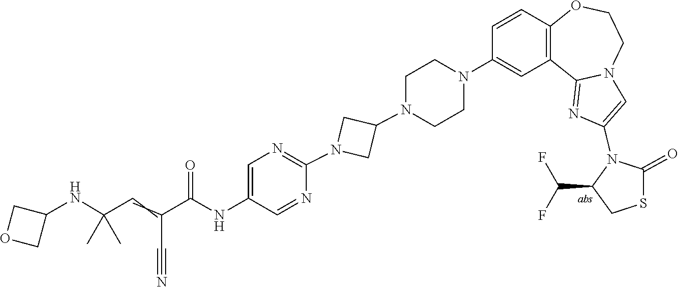 [SMILES: CC(C)(C=C(C#N)C(=O)Nc1cnc(N2CC(N3CCN(c4ccc5c(c4)-c4nc(N6C(=O)SC[C@H]6C(F)F)cn4CCO5)CC3)C2)nc1)NC1COC1] | 746 |
| 258 | 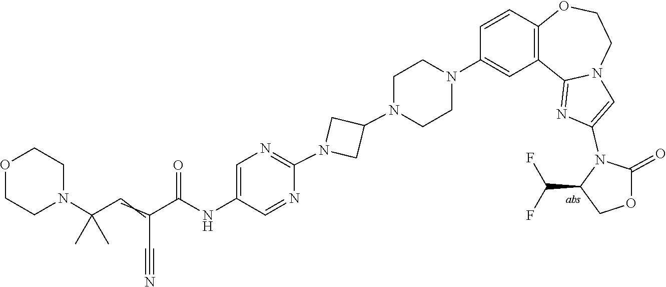 [SMILES: CC(C)(C=C(C#N)C(=O)Nc1cnc(N2CC(N3CCN(c4ccc5c(c4)-c4nc(N6C(=O)OC[C@H]6C(F)F)cn4CCO5)CC3)C2)nc1)N1CCOCC1] | 760 |
| 259 | 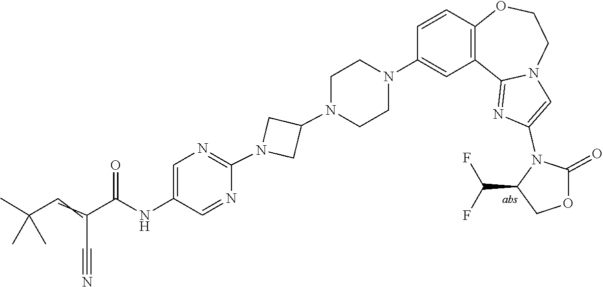 [SMILES: CC(C)(C)C=C(C#N)C(=O)Nc1cnc(N2CC(N3CCN(c4ccc5c(c4)-c4nc(N6C(=O)OC[C@H]6C(F)F)cn4CCO5)CC3)C2)nc1] | 689 |
| 260 | 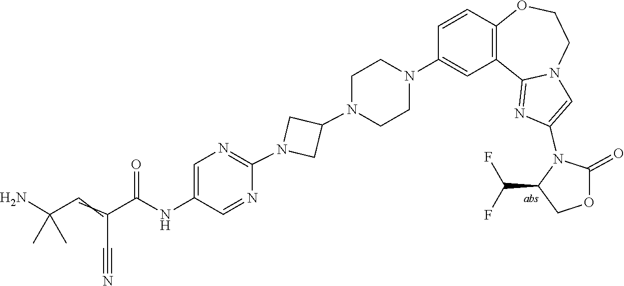 [SMILES: CC(C)(N)C=C(C#N)C(=O)Nc1cnc(N2CC(N3CCN(c4ccc5c(c4)-c4nc(N6C(=O)OC[C@H]6C(F)F)cn4CCO5)CC3)C2)nc1] | 690 |
| 261 | 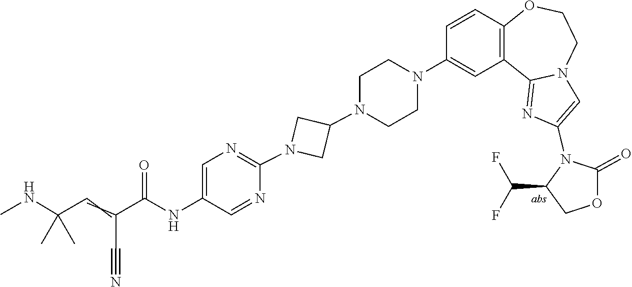 [SMILES: CNC(C)(C)C=C(C#N)C(=O)Nc1cnc(N2CC(N3CCN(c4ccc5c(c4)-c4nc(N6C(=O)OC[C@H]6C(F)F)cn4CCO5)CC3)C2)nc1] | 704 |
| 262 | 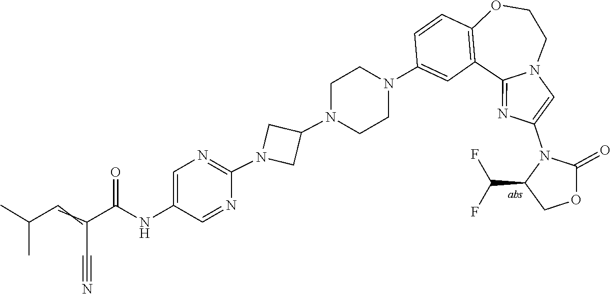 [SMILES: CC(C)C=C(C#N)C(=O)Nc1cnc(N2CC(N3CCN(c4ccc5c(c4)-c4nc(N6C(=O)OC[C@H]6C(F)F)cn4CCO5)CC3)C2)nc1] | 675 |
| 263 | 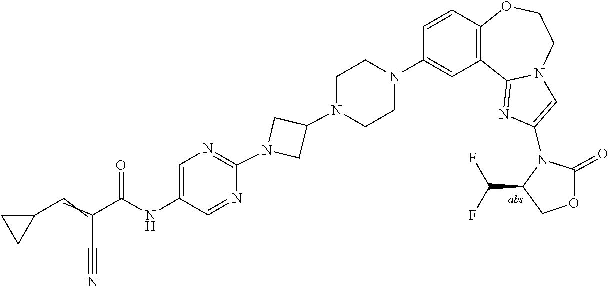 [SMILES: N#CC(=CC1CC1)C(=O)Nc1cnc(N2CC(N3CCN(c4ccc5c(c4)-c4nc(N6C(=O)OC[C@H]6C(F)F)cn4CCO5)CC3)C2)nc1] | 673 |
| 264 | 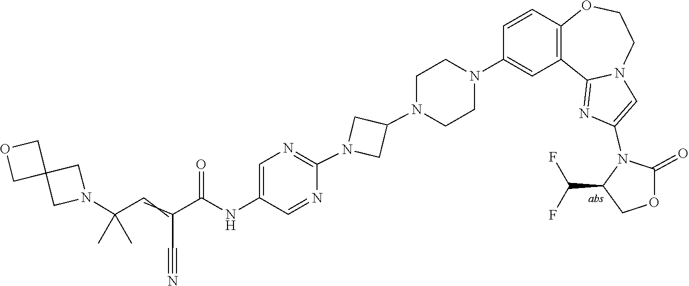 [SMILES: CC(C)(C=C(C#N)C(=O)Nc1cnc(N2CC(N3CCN(c4ccc5c(c4)-c4nc(N6C(=O)OC[C@H]6C(F)F)cn4CCO5)CC3)C2)nc1)N1CC2(COC2)C1] | 772 |
| 265 | 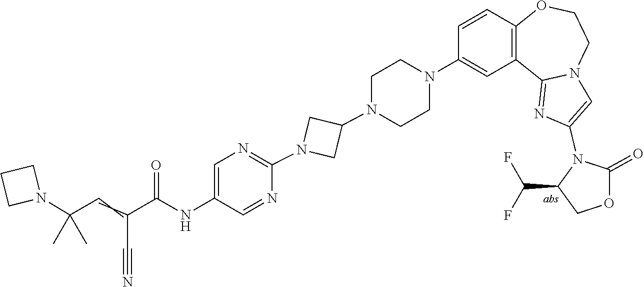 [SMILES: CC(C)(C=C(C#N)C(=O)Nc1cnc(N2CC(N3CCN(c4ccc5c(c4)-c4nc(N6C(=O)OC[C@H]6C(F)F)cn4CCO5)CC3)C2)nc1)N1CCC1] | 730 |
| 266 | 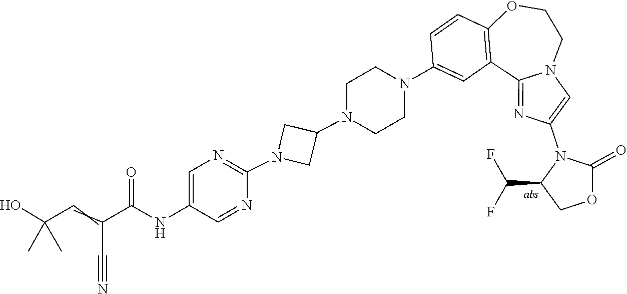 [SMILES: CC(C)(O)C=C(C#N)C(=O)Nc1cnc(N2CC(N3CCN(c4ccc5c(c4)-c4nc(N6C(=O)OC[C@H]6C(F)F)cn4CCO5)CC3)C2)nc1] | 691 |
| 267 | 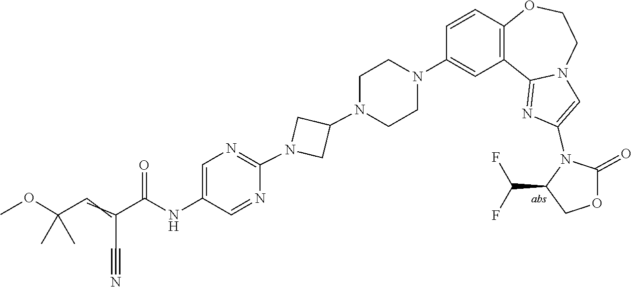 [SMILES: COC(C)(C)C=C(C#N)C(=O)Nc1cnc(N2CC(N3CCN(c4ccc5c(c4)-c4nc(N6C(=O)OC[C@H]6C(F)F)cn4CCO5)CC3)C2)nc1] | 705 |
| 268 | 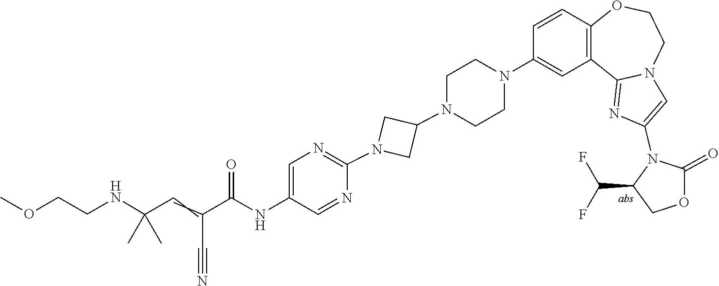 [SMILES: COCCNC(C)(C)C=C(C#N)C(=O)Nc1cnc(N2CC(N3CCN(c4ccc5c(c4)-c4nc(N6C(=O)OC[C@H]6C(F)F)cn4CCO5)CC3)C2)nc1] | 748 |
| 269 | 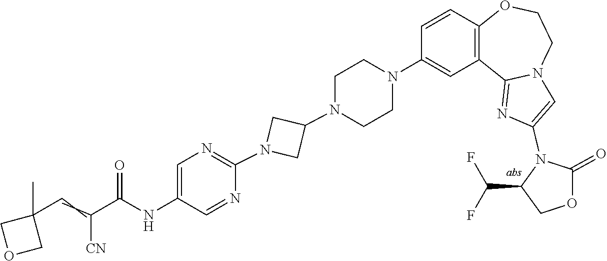 [SMILES: CC1(C=C(C#N)C(=O)Nc2cnc(N3CC(N4CCN(c5ccc6c(c5)-c5nc(N7C(=O)OC[C@H]7C(F)F)cn5CCO6)CC4)C3)nc2)COC1] | 703 |
| 270 | 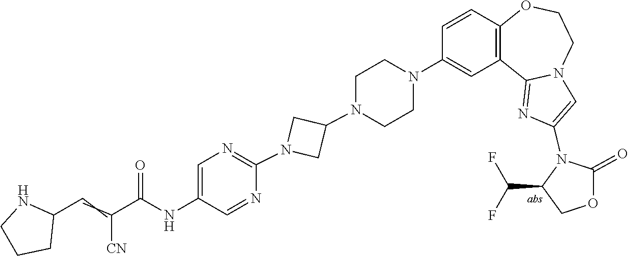 [SMILES: N#CC(=CC1CCCN1)C(=O)Nc1cnc(N2CC(N3CCN(c4ccc5c(c4)-c4nc(N6C(=O)OC[C@H]6C(F)F)cn4CCO5)CC3)C2)nc1] | 702 |
| 271 | 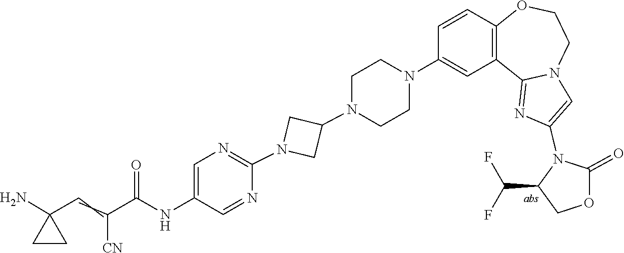 [SMILES: N#CC(=CC1(N)CC1)C(=O)Nc1cnc(N2CC(N3CCN(c4ccc5c(c4)-c4nc(N6C(=O)OC[C@H]6C(F)F)cn4CCO5)CC3)C2)nc1] | 688 |
| 272 | 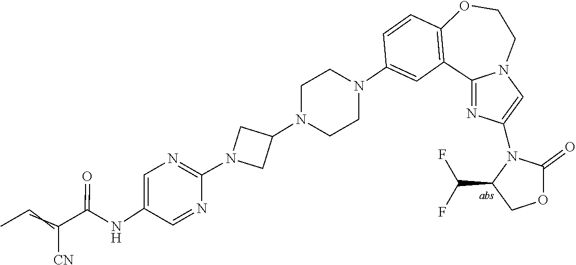 [SMILES: CC=C(C#N)C(=O)Nc1cnc(N2CC(N3CCN(c4ccc5c(c4)-c4nc(N6C(=O)OC[C@H]6C(F)F)cn4CCO5)CC3)C2)nc1] | 647 |
| 273 | 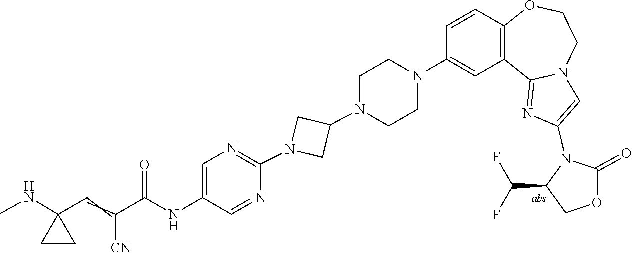 [SMILES: CNC1(C=C(C#N)C(=O)Nc2cnc(N3CC(N4CCN(c5ccc6c(c5)-c5nc(N7C(=O)OC[C@H]7C(F)F)cn5CCO6)CC4)C3)nc2)CC1] | 702 |
| 274 | 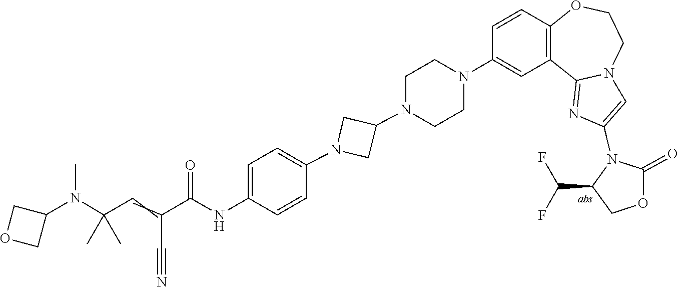 [SMILES: CN(C1COC1)C(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1] | 758 |
| 275A | 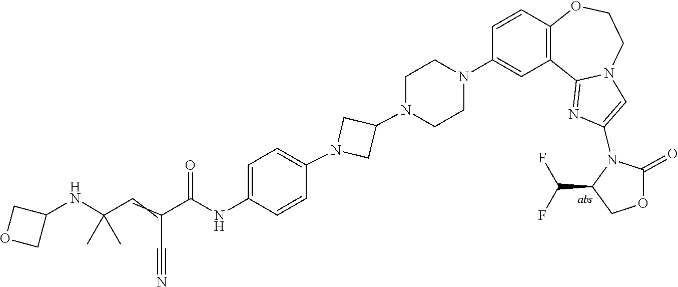 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)NC1COC1] | 744 |
| 275B | 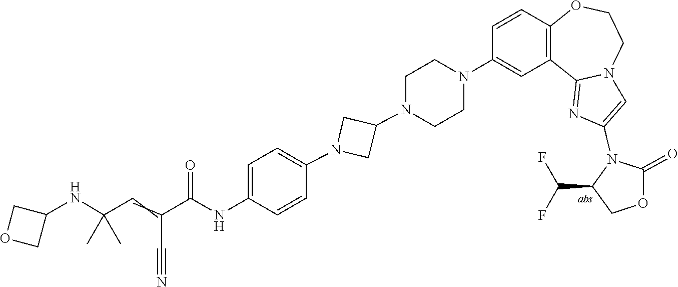 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)NC1COC1] | 744 |
| 276 | 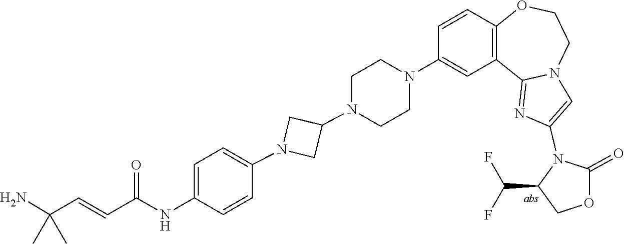 [SMILES: CC(C)(N)/C=C/C(=O)Nc1ccc(N2CC(N3CCN(c4ccc5c(c4)-c4nc(N6C(=O)OC[C@H]6C(F)F)cn4CCO5)CC3)C2)cc1] | 663 |
| 277 | 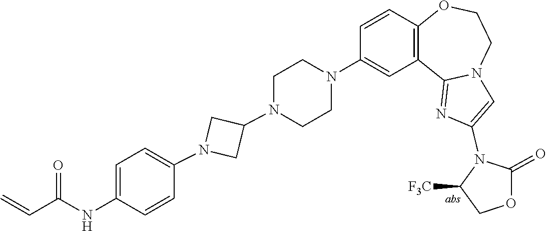 [SMILES: C=CC(=O)Nc1ccc(N2CC(N3CCN(c4ccc5c(c4)-c4nc(N6C(=O)OC[C@H]6C(F)(F)F)cn4CCO5)CC3)C2)cc1] | 624 |
| 278 | 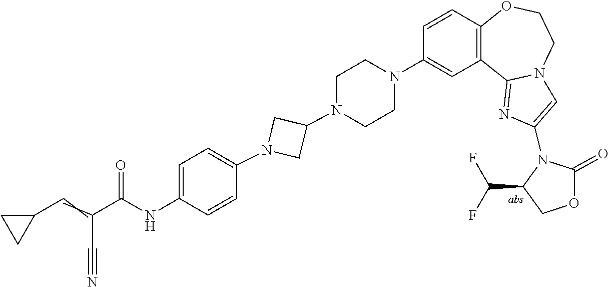 [SMILES: N#CC(=CC1CC1)C(=O)Nc1ccc(N2CC(N3CCN(c4ccc5c(c4)-c4nc(N6C(=O)OC[C@H]6C(F)F)cn4CCO5)CC3)C2)cc1] | 671 |
| 279 | 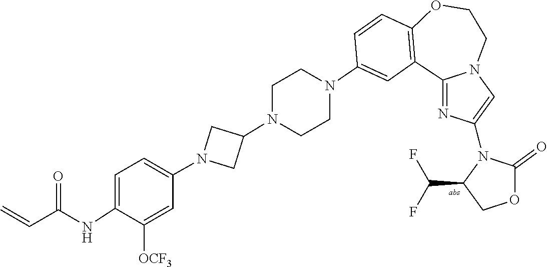 [SMILES: C=CC(=O)Nc1ccc(N2CC(N3CCN(c4ccc5c(c4)-c4nc(N6C(=O)OC[C@H]6C(F)F)cn4CCO5)CC3)C2)cc1OC(F)(F)F] | 690 |
| 280 | 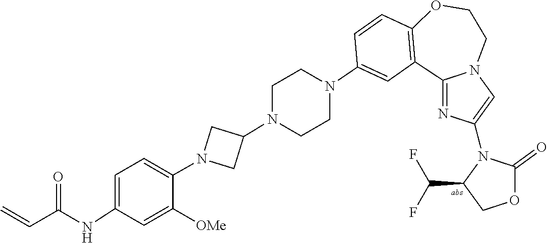 [SMILES: C=CC(=O)Nc1ccc(N2CC(N3CCN(c4ccc5c(c4)-c4nc(N6C(=O)OC[C@H]6C(F)F)cn4CCO5)CC3)C2)c(OC)c1] | 636 |
| 281 | 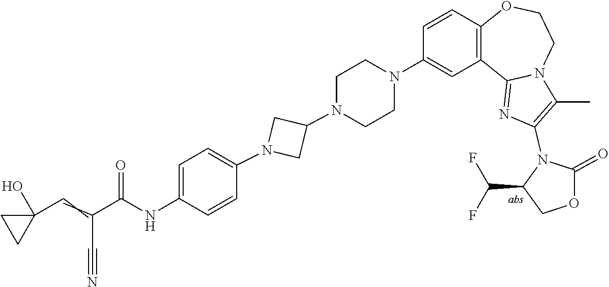 [SMILES: Cc1c(N2C(=O)OC[C@H]2C(F)F)nc2n1CCOc1ccc(N3CCN(C4CN(c5ccc(NC(=O)C(C#N)=CC6(O)CC6)cc5)C4)CC3)cc1-2] | 687 |
| 282 | 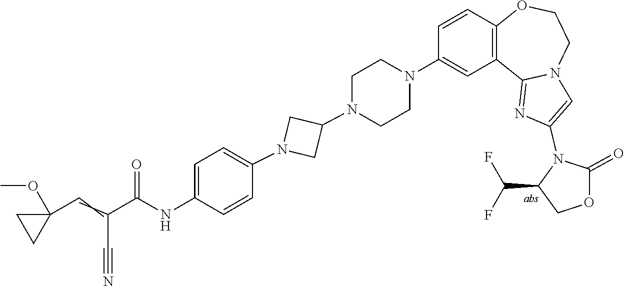 [SMILES: COC1(C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CC1] | 701 |
| 283 | 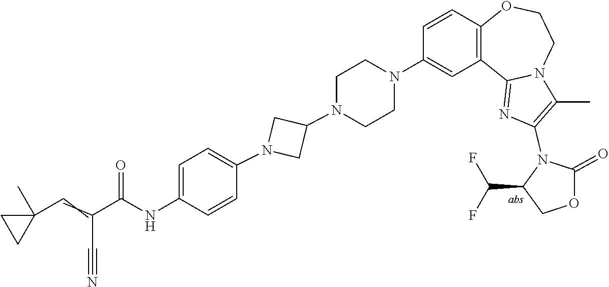 [SMILES: Cc1c(N2C(=O)OC[C@H]2C(F)F)nc2n1CCOc1ccc(N3CCN(C4CN(c5ccc(NC(=O)C(C#N)=CC6(C)CC6)cc5)C4)CC3)cc1-2] | 685 |
| 284 | 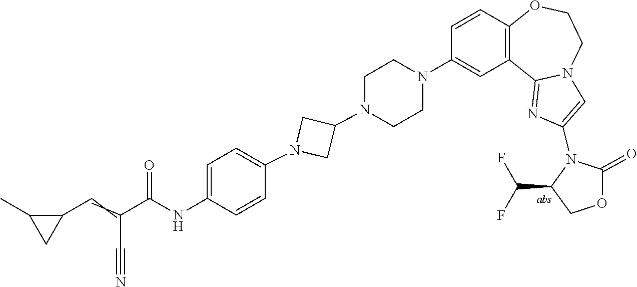 [SMILES: CC1CC1C=C(C#N)C(=O)Nc1ccc(N2CC(N3CCN(c4ccc5c(c4)-c4nc(N6C(=O)OC[C@H]6C(F)F)cn4CCO5)CC3)C2)cc1] | 685 |
| 285 | 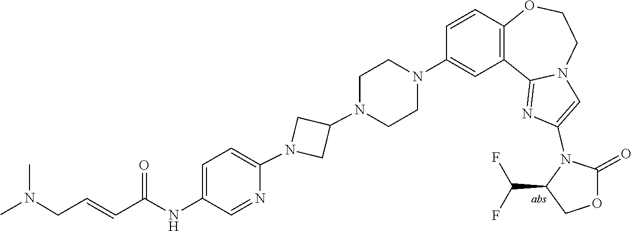 [SMILES: CN(C)C/C=C/C(=O)Nc1ccc(N2CC(N3CCN(c4ccc5c(c4)-c4nc(N6C(=O)OC[C@H]6C(F)F)cn4CCO5)CC3)C2)nc1] | 664 |
| 286 | 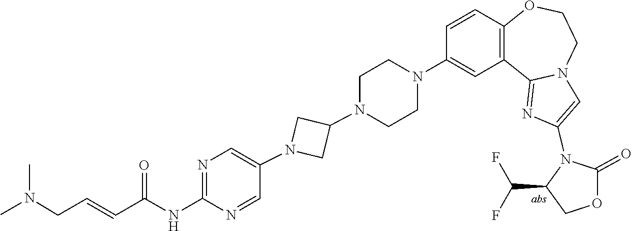 [SMILES: CN(C)C/C=C/C(=O)Nc1ncc(N2CC(N3CCN(c4ccc5c(c4)-c4nc(N6C(=O)OC[C@H]6C(F)F)cn4CCO5)CC3)C2)cn1] | 665 |
| 287 | 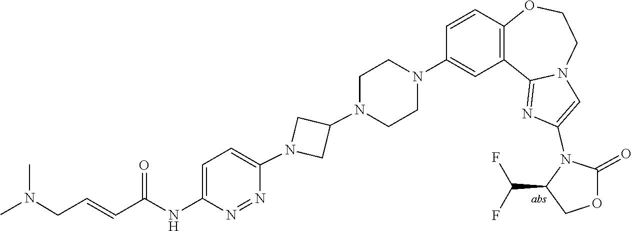 [SMILES: CN(C)C/C=C/C(=O)Nc1ccc(N2CC(N3CCN(c4ccc5c(c4)-c4nc(N6C(=O)OC[C@H]6C(F)F)cn4CCO5)CC3)C2)nn1] | 665 |
| 288 | 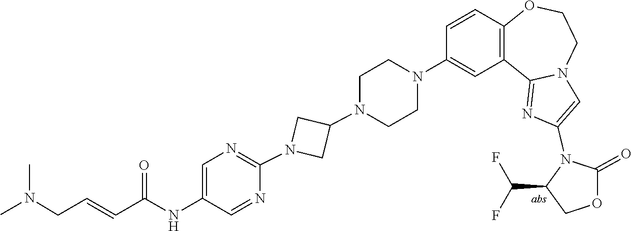 [SMILES: CN(C)C/C=C/C(=O)Nc1cnc(N2CC(N3CCN(c4ccc5c(c4)-c4nc(N6C(=O)OC[C@H]6C(F)F)cn4CCO5)CC3)C2)nc1] | 665 |
| 289 | 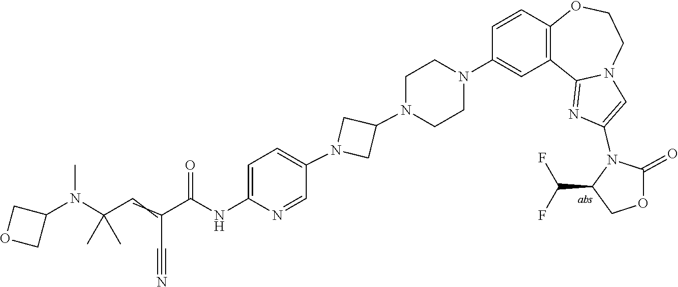 [SMILES: CN(C1COC1)C(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cn1] | 759 |
| 290 | 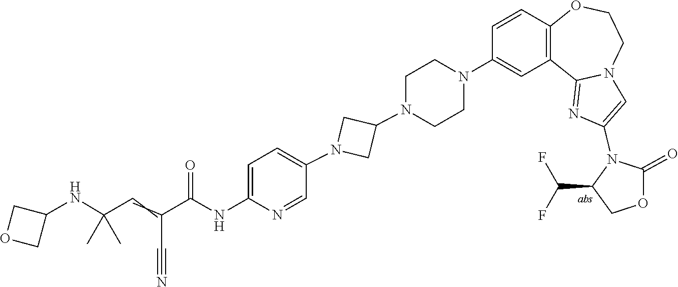 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cn1)NC1COC1] | 745 |
| 291 | 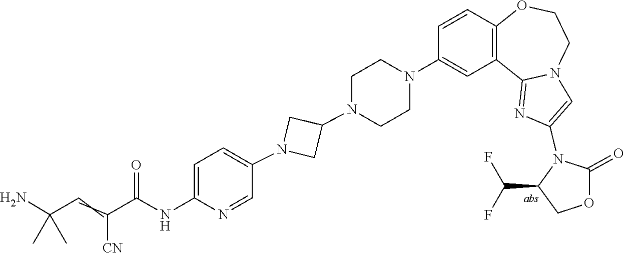 [SMILES: CC(C)(N)C=C(C#N)C(=O)Nc1ccc(N2CC(N3CCN(c4ccc5c(c4)-c4nc(N6C(=O)OC[C@H]6C(F)F)cn4CCO5)CC3)C2)cn1] | 689 |
| 292 | 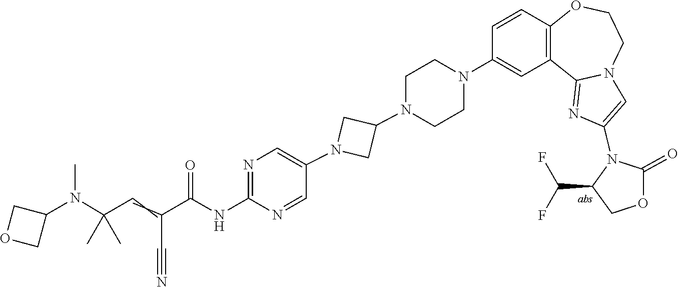 [SMILES: CN(C1COC1)C(C)(C)C=C(C#N)C(=O)Nc1ncc(N2CC(N3CCN(c4ccc5c(c4)-c4nc(N6C(=O)OC[C@H]6C(F)F)cn4CCO5)CC3)C2)cn1] | 760 |
| 293 | 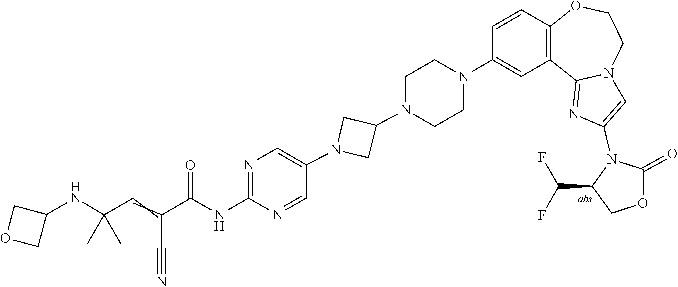 [SMILES: CC(C)(C=C(C#N)C(=O)Nc1ncc(N2CC(N3CCN(c4ccc5c(c4)-c4nc(N6C(=O)OC[C@H]6C(F)F)cn4CCO5)CC3)C2)cn1)NC1COC1] | 746 |
| 294 | 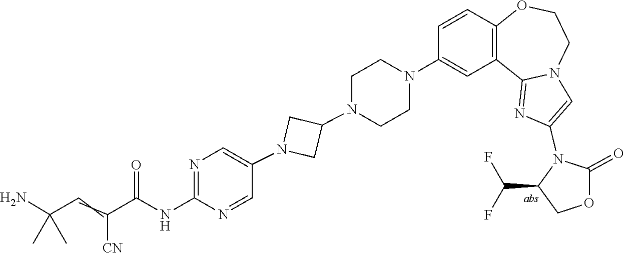 [SMILES: CC(C)(N)C=C(C#N)C(=O)Nc1ncc(N2CC(N3CCN(c4ccc5c(c4)-c4nc(N6C(=O)OC[C@H]6C(F)F)cn4CCO5)CC3)C2)cn1] | 690 |
| 295 | 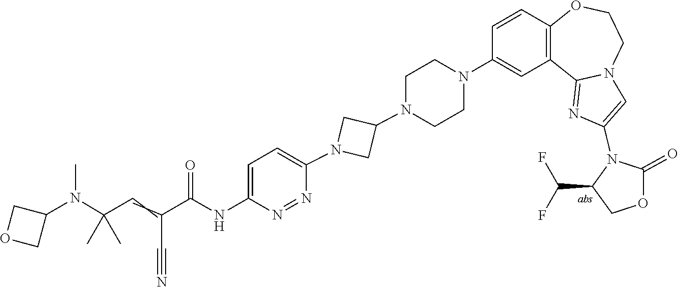 [SMILES: CN(C1COC1)C(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)nn1] | 760 |
| 296 | 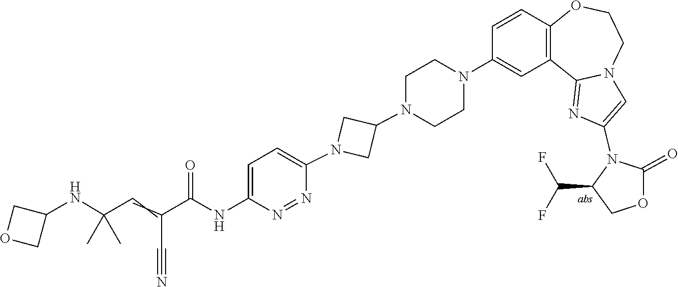 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)nn1)NC1COC1] | 746 |
| 297 | 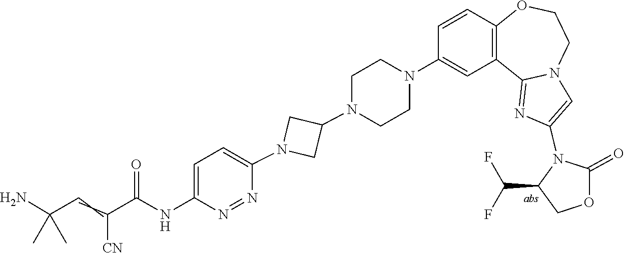 [SMILES: CC(C)(N)C=C(C#N)C(=O)Nc1ccc(N2CC(N3CCN(c4ccc5c(c4)-c4nc(N6C(=O)OC[C@H]6C(F)F)cn4CCO5)CC3)C2)nn1] | 690 |
| 298 | 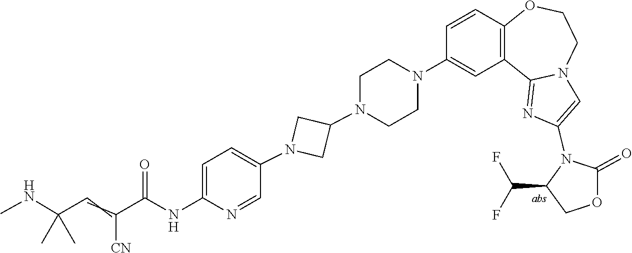 [SMILES: CNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cn1] | 703 |
| 299 | 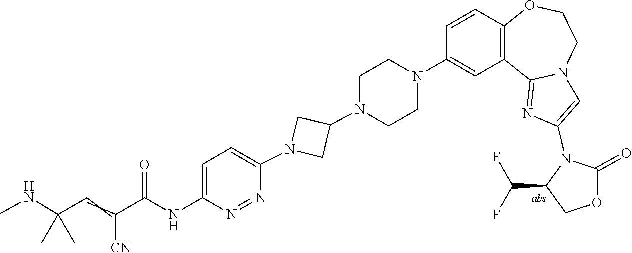 [SMILES: CNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)nn1] | 704 |
| 300 | 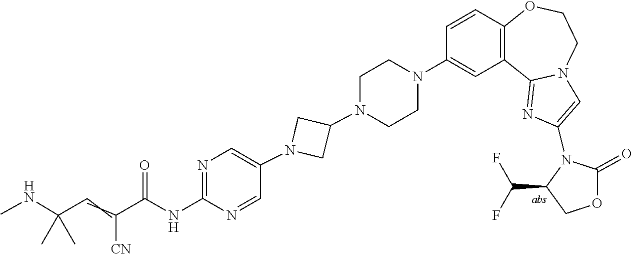 [SMILES: CNC(C)(C)C=C(C#N)C(=O)Nc1ncc(N2CC(N3CCN(c4ccc5c(c4)-c4nc(N6C(=O)OC[C@H]6C(F)F)cn4CCO5)CC3)C2)cn1] | 704 |
| 301 | 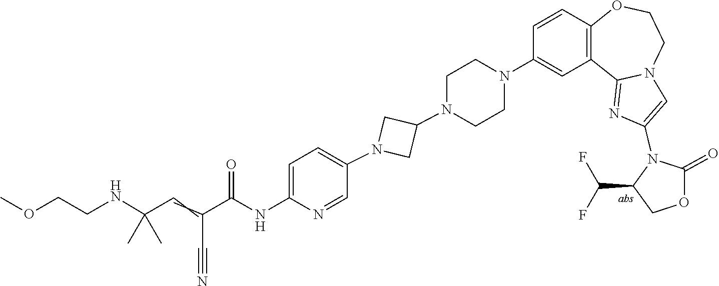 [SMILES: COCCNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cn1] | 747 |
| 302 | 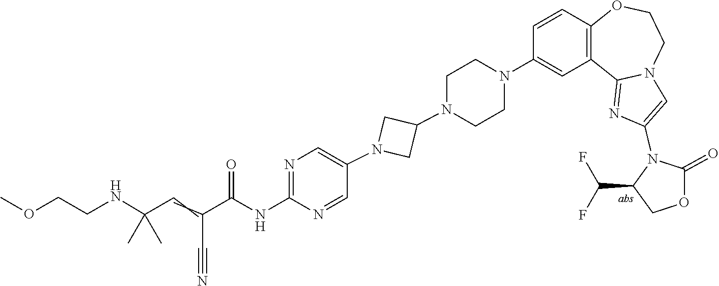 [SMILES: COCCNC(C)(C)C=C(C#N)C(=O)Nc1ncc(N2CC(N3CCN(c4ccc5c(c4)-c4nc(N6C(=O)OC[C@H]6C(F)F)cn4CCO5)CC3)C2)cn1] | 748 |
| 303 | 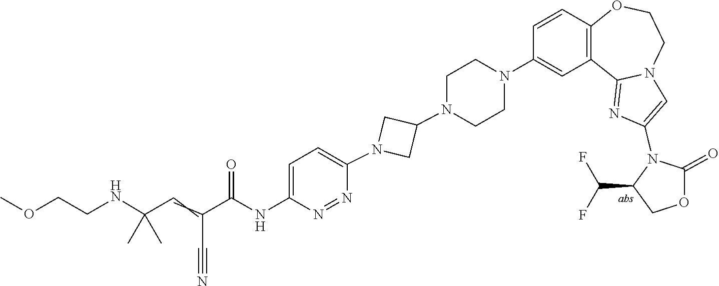 [SMILES: COCCNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)nn1] | 748 |
| 304 | 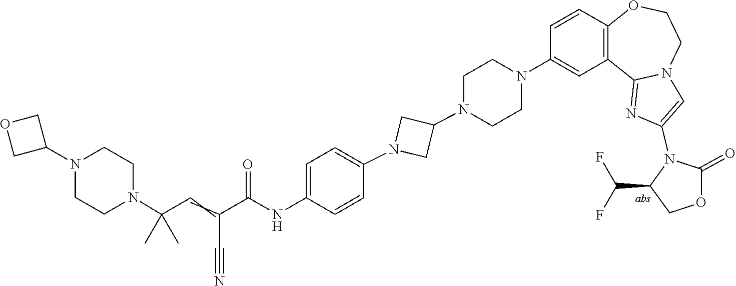 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)N1CCN(C2COC2)CC1] | 813 |
| 305 | 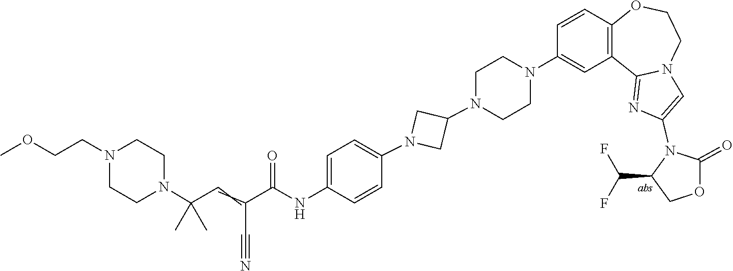 [SMILES: COCCN1CCN(C(C)(C)C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CC1] | 815 |
| 306 | 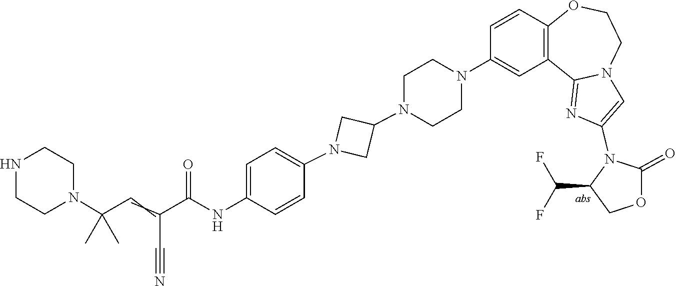 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)N1CCNCC1] | 757 |
| 307 | 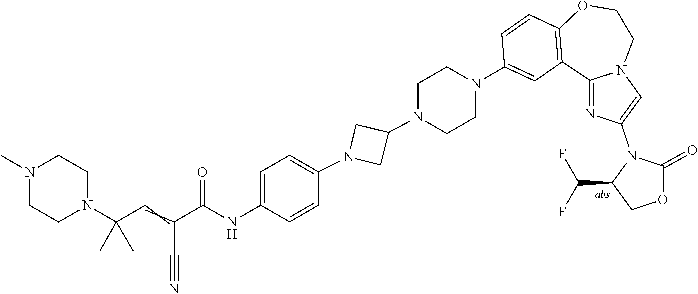 [SMILES: CN1CCN(C(C)(C)C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CC1] | 771 |
| 308 | 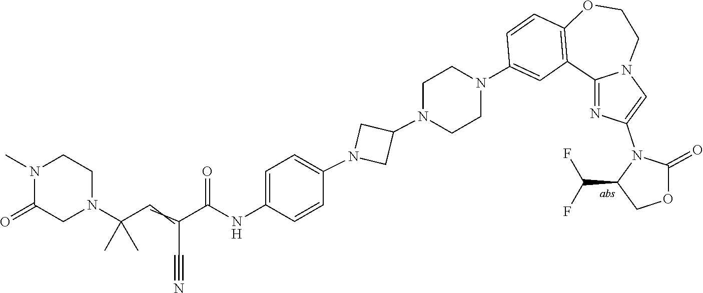 [SMILES: CN1CCN(C(C)(C)C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CC1=O] | 785 |
| 309 | 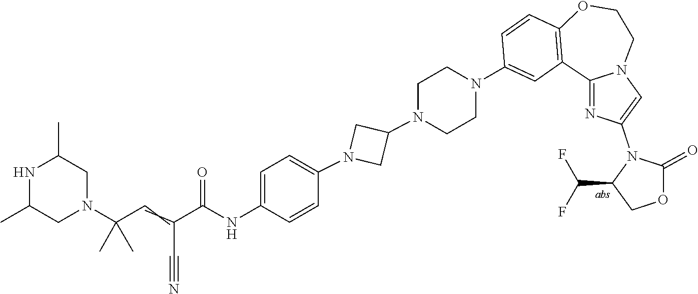 [SMILES: CC1CN(C(C)(C)C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CC(C)N1] | 785 |
| 310 | 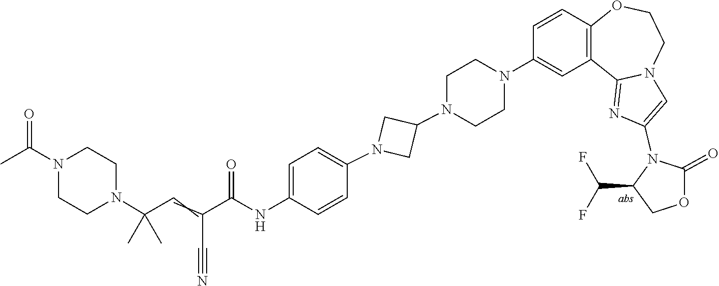 [SMILES: CC(=O)N1CCN(C(C)(C)C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CC1] | 799 |
| 311 | 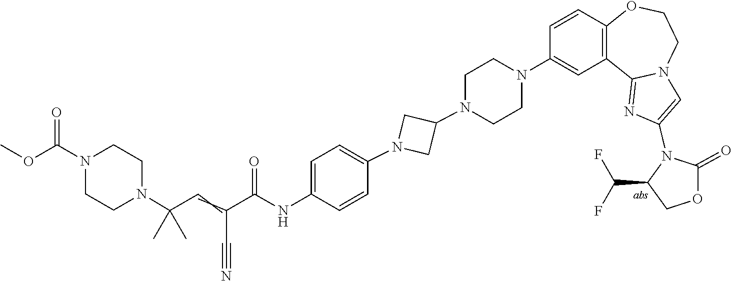 [SMILES: COC(=O)N1CCN(C(C)(C)C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CC1] | 815 |
| 312 | 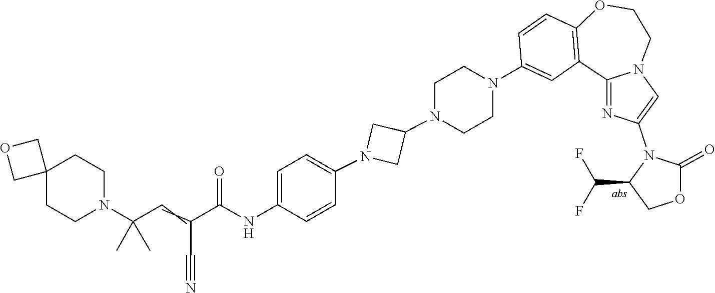 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)N1CCC2(CC1)COC2] | 798 |
| 313 | 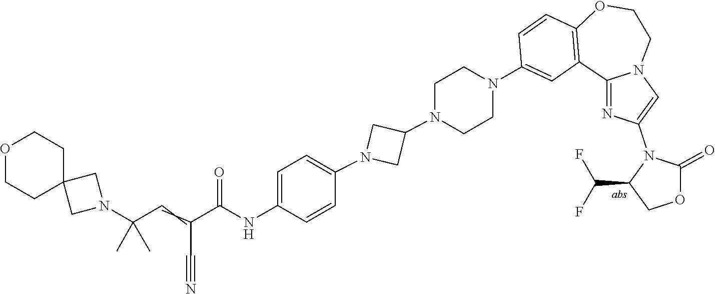 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)N1CC2(CCOCC2)C1] | 798 |
| 314 | 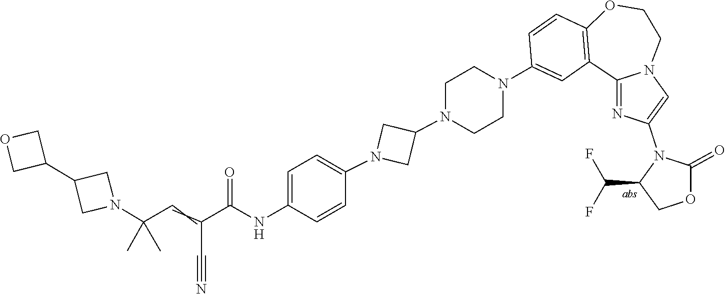 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)N1CC(C2COC2)C1] | 784 |
| 315 | 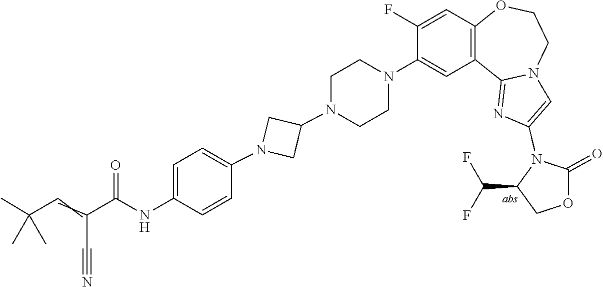 [SMILES: CC(C)(C)C=C(C#N)C(=O)Nc1ccc(N2CC(N3CCN(c4cc5c(cc4F)OCCn4cc(N6C(=O)OC[C@H]6C(F)F)nc4-5)CC3)C2)cc1] | 705 |
| 316 | 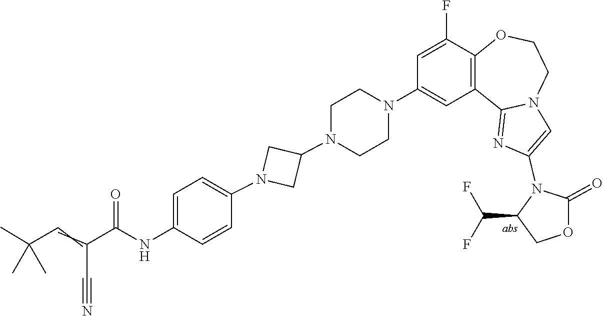 [SMILES: CC(C)(C)C=C(C#N)C(=O)Nc1ccc(N2CC(N3CCN(c4cc(F)c5c(c4)-c4nc(N6C(=O)OC[C@H]6C(F)F)cn4CCO5)CC3)C2)cc1] | 705 |
| 317 | 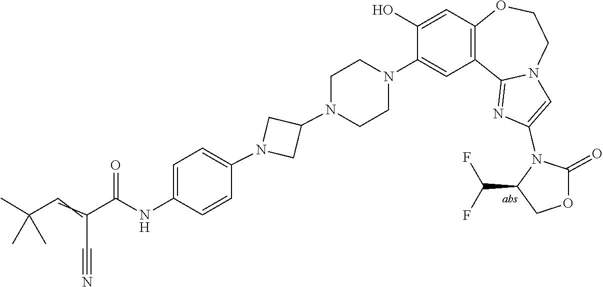 [SMILES: CC(C)(C)C=C(C#N)C(=O)Nc1ccc(N2CC(N3CCN(c4cc5c(cc4O)OCCn4cc(N6C(=O)OC[C@H]6C(F)F)nc4-5)CC3)C2)cc1] | 703 |
| 318 | 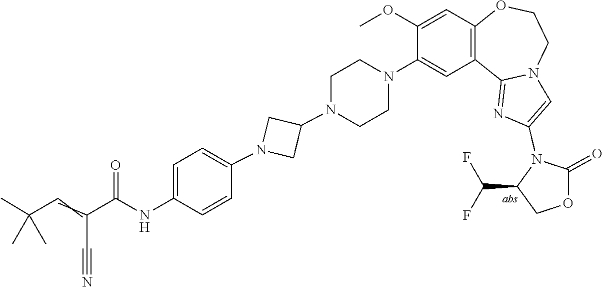 [SMILES: COc1cc2c(cc1N1CCN(C3CN(c4ccc(NC(=O)C(C#N)=CC(C)(C)C)cc4)C3)CC1)-c1nc(N3C(=O)OC[C@H]3C(F)F)cn1CCO2] | 717 |
| 319 | 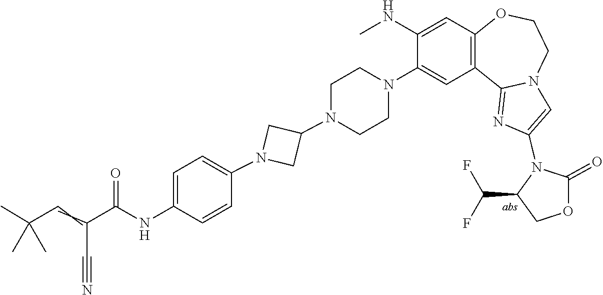 [SMILES: CNc1cc2c(cc1N1CCN(C3CN(c4ccc(NC(=O)C(C#N)=CC(C)(C)C)cc4)C3)CC1)-c1nc(N3C(=O)OC[C@H]3C(F)F)cn1CCO2] | 716 |
| 320 | 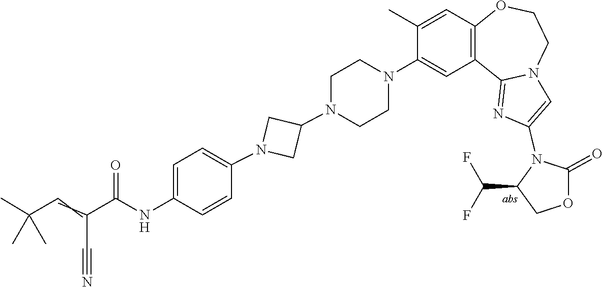 [SMILES: Cc1cc2c(cc1N1CCN(C3CN(c4ccc(NC(=O)C(C#N)=CC(C)(C)C)cc4)C3)CC1)-c1nc(N3C(=O)OC[C@H]3C(F)F)cn1CCO2] | 701 |
| 321 | 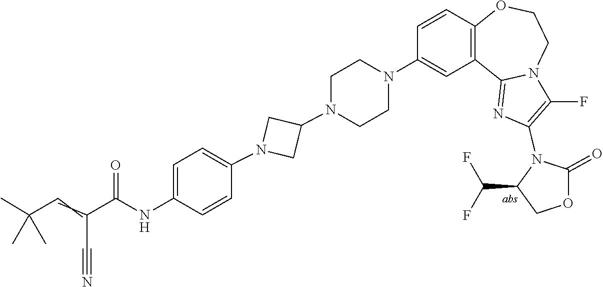 [SMILES: CC(C)(C)C=C(C#N)C(=O)Nc1ccc(N2CC(N3CCN(c4ccc5c(c4)-c4nc(N6C(=O)OC[C@H]6C(F)F)c(F)n4CCO5)CC3)C2)cc1] | 705 |
| 322 | 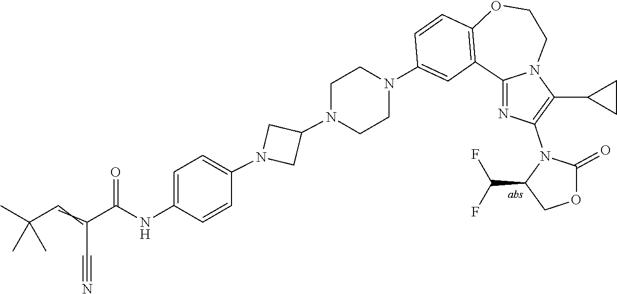 [SMILES: CC(C)(C)C=C(C#N)C(=O)Nc1ccc(N2CC(N3CCN(c4ccc5c(c4)-c4nc(N6C(=O)OC[C@H]6C(F)F)c(C6CC6)n4CCO5)CC3)C2)cc1] | 727 |
| 323 | 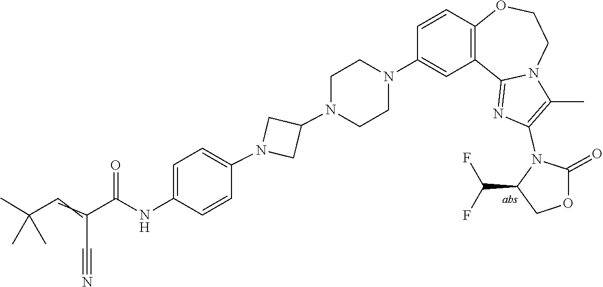 [SMILES: Cc1c(N2C(=O)OC[C@H]2C(F)F)nc2n1CCOc1ccc(N3CCN(C4CN(c5ccc(NC(=O)C(C#N)=CC(C)(C)C)cc5)C4)CC3)cc1-2] | 701 |
| 324A | 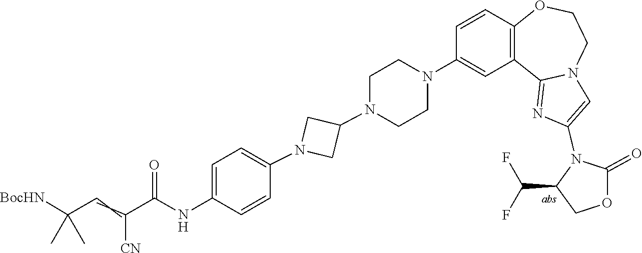 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)NC(=O)OC(C)(C)C] | 788 |
| | [symbol] | |
| 324B | 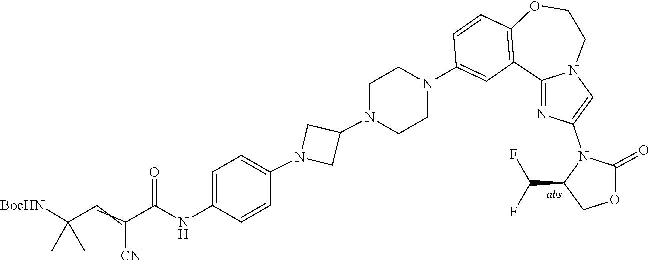 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)NC(=O)OC(C)(C)C] | 788 |
| | [symbol] | |
| 325 | 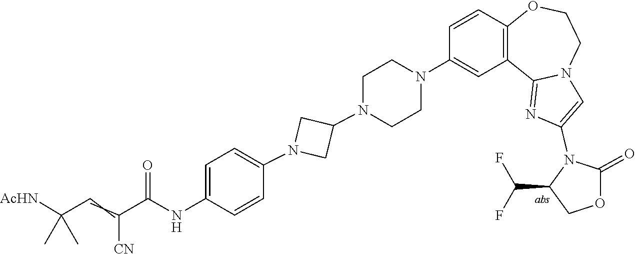 [SMILES: CC(=O)NC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1] | 730 |
| 326 | 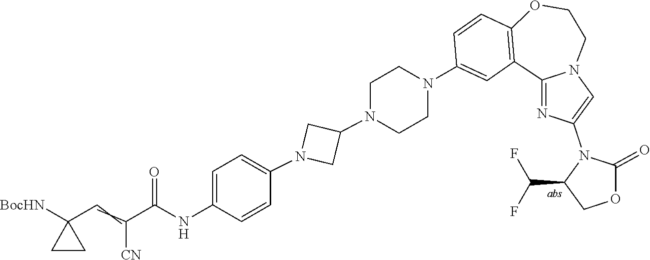 [SMILES: CC(C)(C)OC(=O)NC1(C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CC1] | 786 |
| 327 | 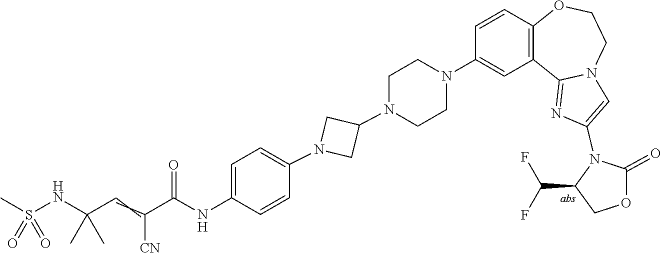 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)NS(C)(=O)=O] | 766 |
| 328 | 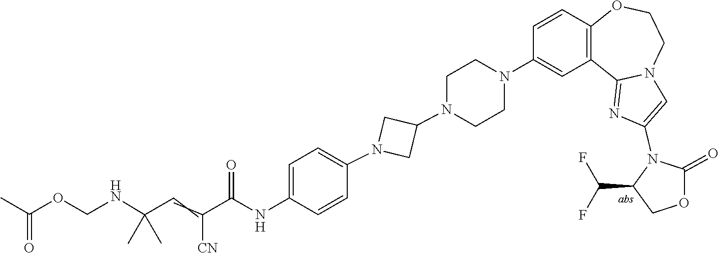 [SMILES: CC(=O)OCNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1] | 760 |
| 329 | 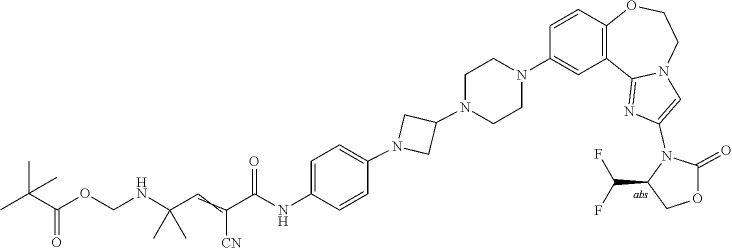 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)NCOC(=O)C(C)(C)C] | 802 |
| 330 | 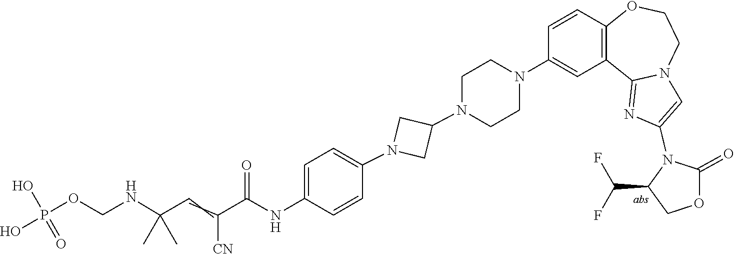 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)NCOP(=O)(O)O] | 780 |
| 331 | 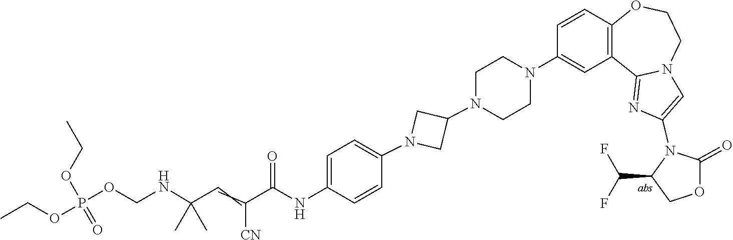 [SMILES: CCOP(=O)(OCC)OCNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1] | 854 |
| 332 | 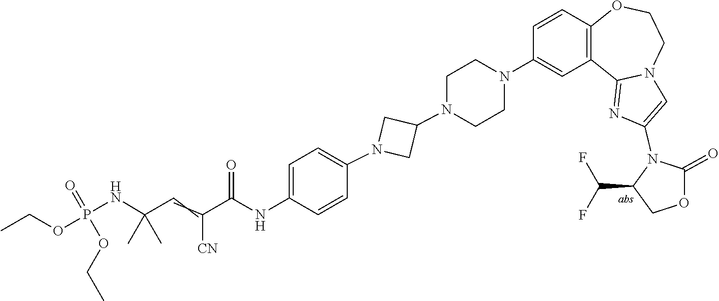 [SMILES: CCOP(=O)(NC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1)OCC] | 824 |
| 333 | 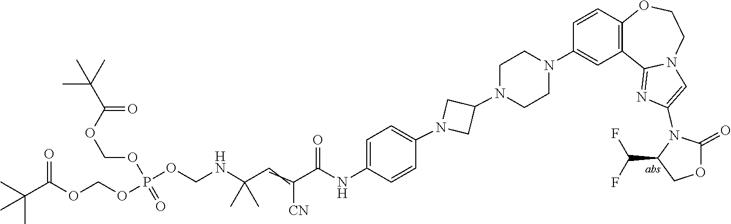 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)NCOP(=O)(OCOC(=O)C(C)(C)C)OCOC(=O)C(C)(C)C] | 1026 |
| 334 | 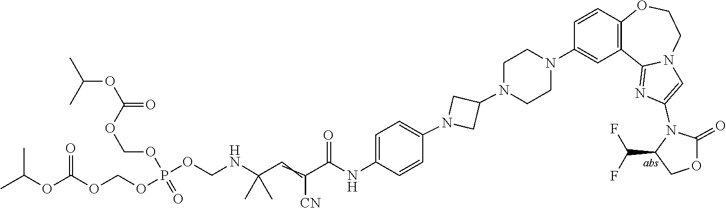 [SMILES: CC(C)OC(=O)OCOP(=O)(OCNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1)OCOC(=O)OC(C)C] | 1030 |
| 335 | 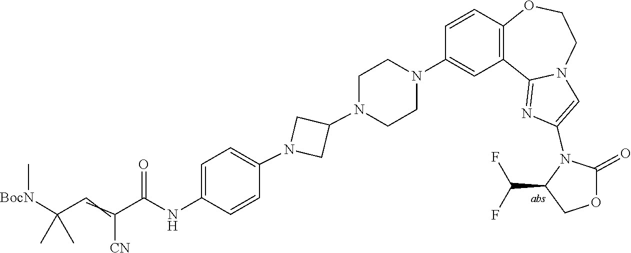 [SMILES: CN(C(=O)OC(C)(C)C)C(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1] | 802 |
| 336 | 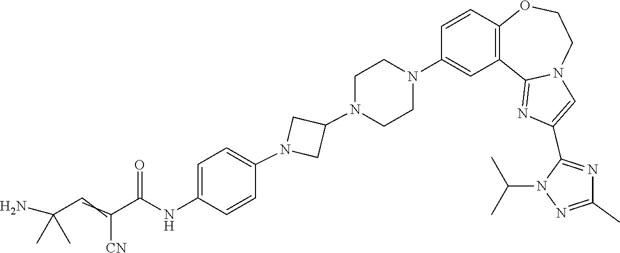 [SMILES: Cc1nc(-c2cn3c(n2)-c2cc(N4CCN(C5CN(c6ccc(NC(=O)C(C#N)=CC(C)(C)N)cc6)C5)CC4)ccc2OCC3)n(C(C)C)n1] | 676 |
| 337 | 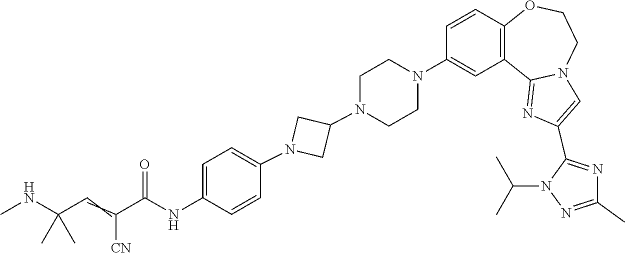 | 690 |
| 338 | 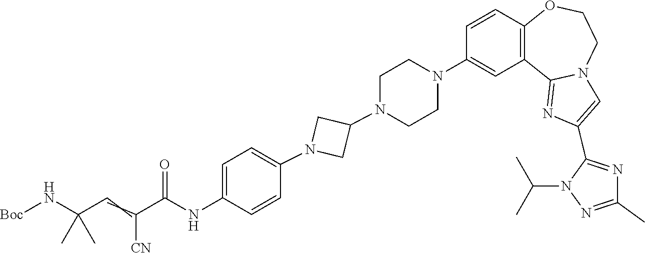 [SMILES: Cc1nc(-c2cn3c(n2)-c2cc(N4CCN(C5CN(c6ccc(NC(=O)C(C#N)=CC(C)(C)NC(=O)OC(C)(C)C)cc6)C5)CC4)ccc2OCC3)n(C(C)C)n1] | 776 |
| 339 | 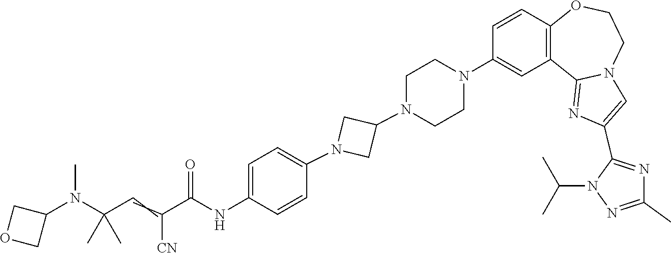 [SMILES: Cc1nc(-c2cn3c(n2)-c2cc(N4CCN(C5CN(c6ccc(NC(=O)C(C#N)=CC(C)(C)N(C)C7COC7)cc6)C5)CC4)ccc2OCC3)n(C(C)C)n1] | 746 |
| 340 | 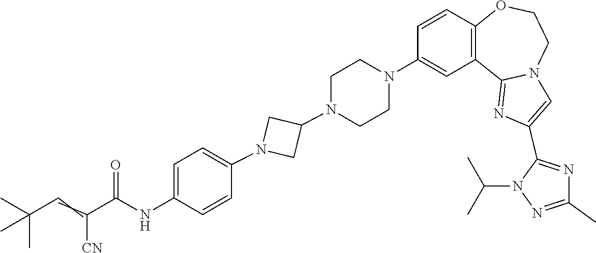 [SMILES: Cc1nc(-c2cn3c(n2)-c2cc(N4CCN(C5CN(c6ccc(NC(=O)C(C#N)=CC(C)(C)C)cc6)C5)CC4)ccc2OCC3)n(C(C)C)n1] | 675 |
| 341 | 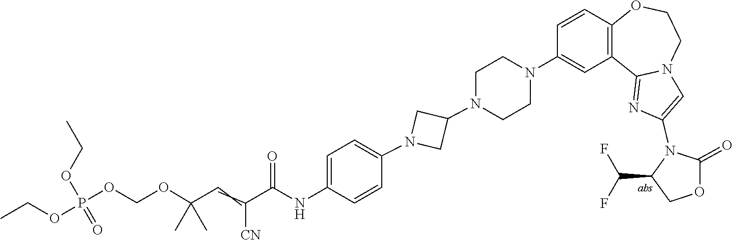 [SMILES: CCOP(=O)(OCC)OCOC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1] | 855 |
| 342 | 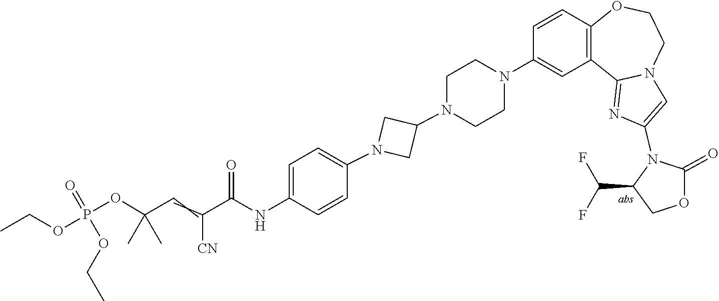 [SMILES: CCOP(=O)(OCC)OC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1] | 825 |
| 343 | 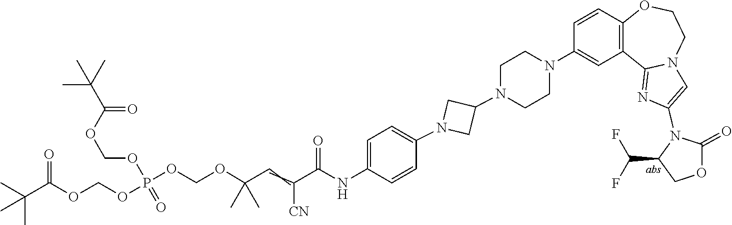 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)OCOP(=O)(OCOC(=O)C(C)(C)C)OCOC(=O)C(C)(C)C] | 1027 |
| 344 | 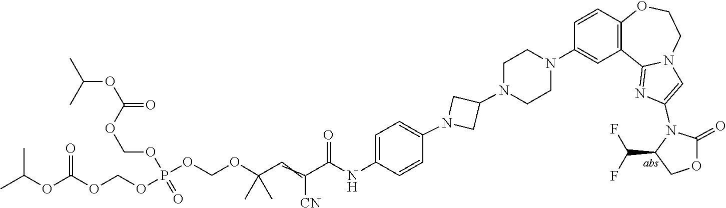 [SMILES: CC(C)OC(=O)OCOP(=O)(OCOC(=O)OC(C)C)OCOC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1] | 1031 |
| 345 | 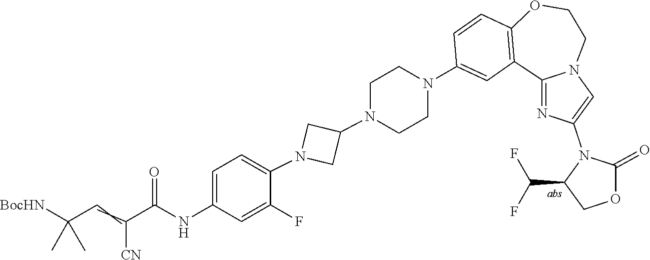 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1)NC(=O)OC(C)(C)C] | 806 |
| 346 | 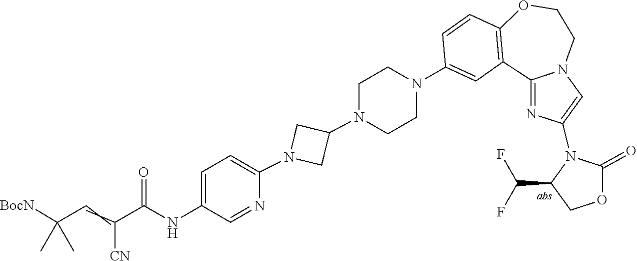 | 789 |
| 347 | 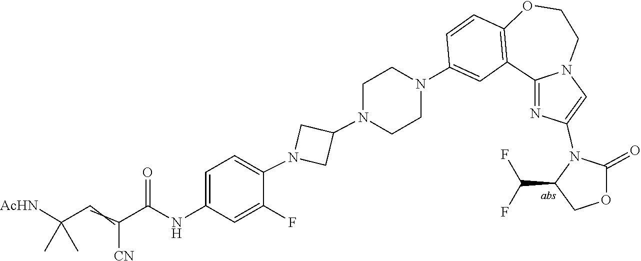 [SMILES: CC(=O)NC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1] | 748 |
| 348 | 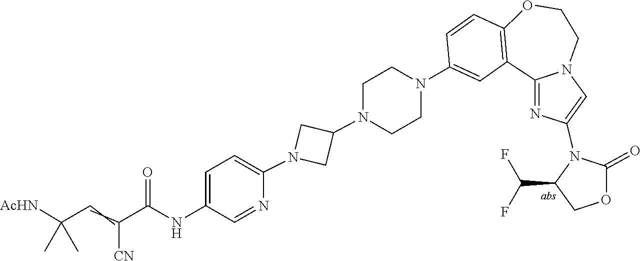 [SMILES: CC(=O)NC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)nc1] | 731 |
| 349 | 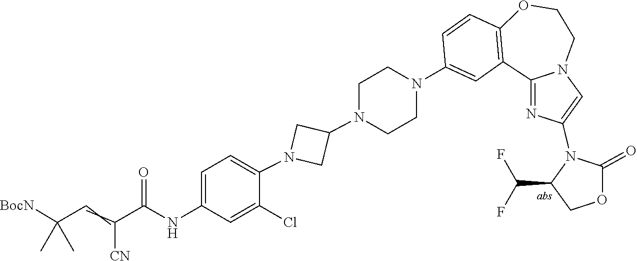 | 822 |
| 350 | 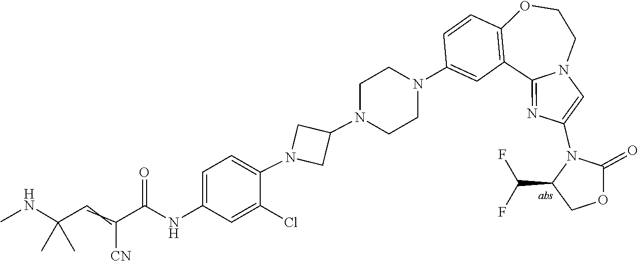 [SMILES: CNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)c(Cl)c1] | 736 |
| 351 | 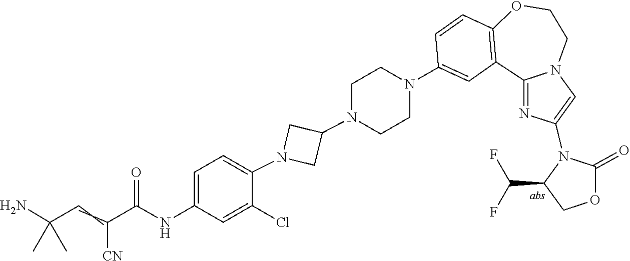 [SMILES: CC(C)(N)C=C(C#N)C(=O)Nc1ccc(N2CC(N3CCN(c4ccc5c(c4)-c4nc(N6C(=O)OC[C@H]6C(F)F)cn4CCO5)CC3)C2)c(Cl)c1] | 722 |
| 352 | 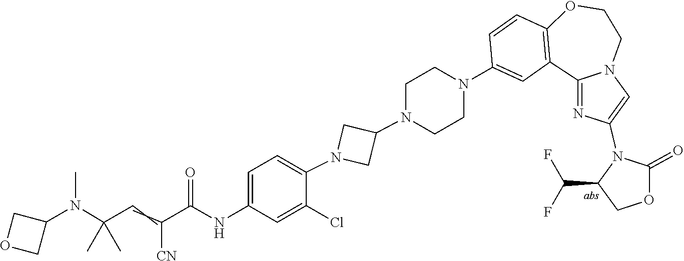 [SMILES: CN(C1COC1)C(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)c(Cl)c1] | 792 |
| 353 | 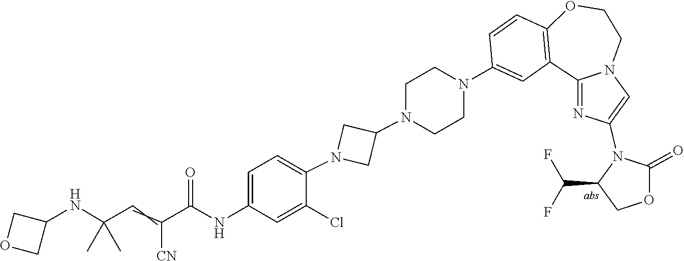 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)c(Cl)c1)NC1COC1] | 778 |
| 354 | 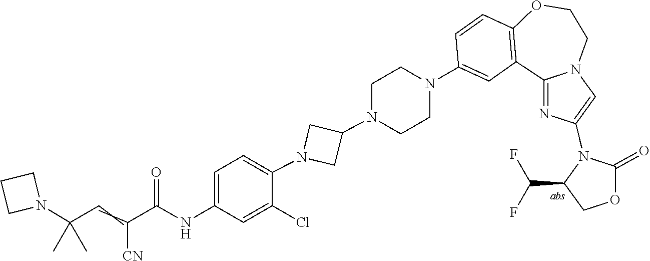 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)c(Cl)c1)N1CCC1] | 762 |
| 355 | 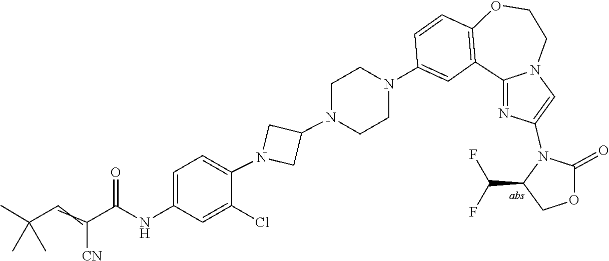 [SMILES: CC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)c(Cl)c1] | 721 |
| 356 | 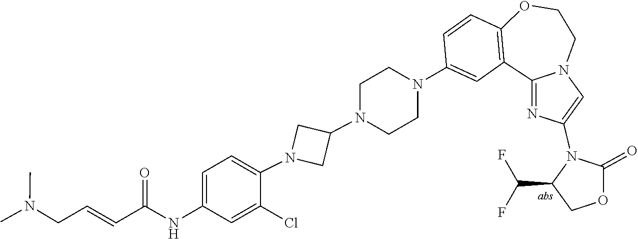 [SMILES: CN(C)C/C=C/C(=O)Nc1ccc(N2CC(N3CCN(c4ccc5c(c4)-c4nc(N6C(=O)OC[C@H]6C(F)F)cn4CCO5)CC3)C2)c(Cl)c1] | 697 |
| 357 | 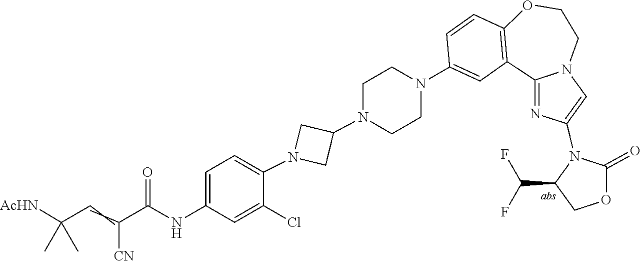 [SMILES: CC(=O)NC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)c(Cl)c1] | 764 |
| 358 | 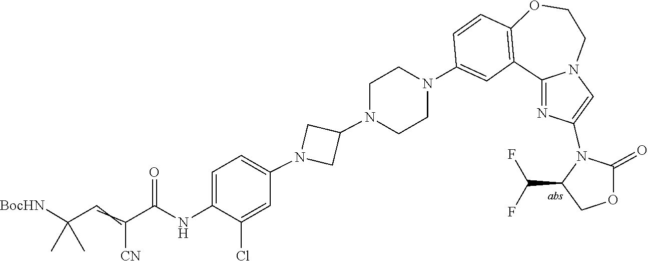 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1Cl)NC(=O)OC(C)(C)C] | 822 |
| 359 | 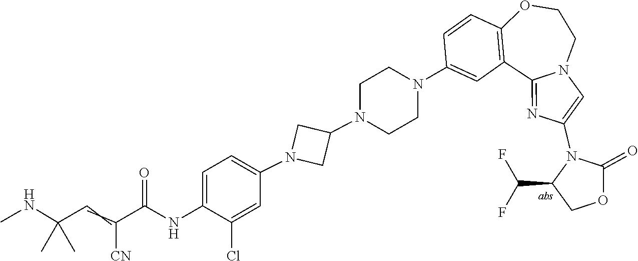 [SMILES: CNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1Cl] | 736 |
| 360 | 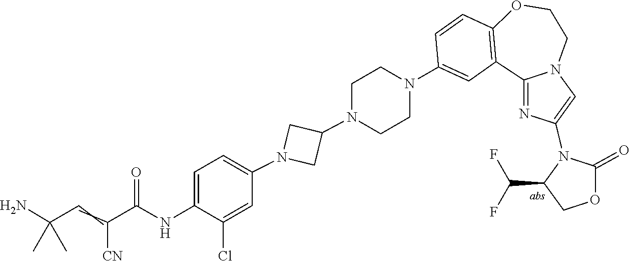 [SMILES: CC(C)(N)C=C(C#N)C(=O)Nc1ccc(N2CC(N3CCN(c4ccc5c(c4)-c4nc(N6C(=O)OC[C@H]6C(F)F)cn4CCO5)CC3)C2)cc1Cl] | 722 |
| 361 | 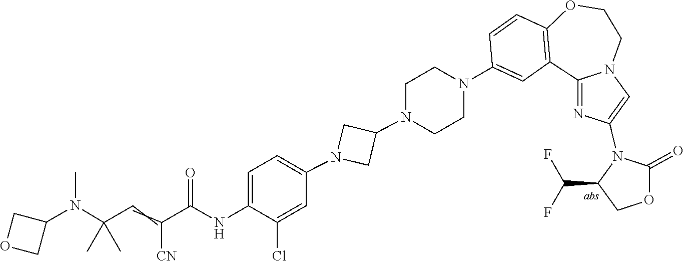 [SMILES: CN(C1COC1)C(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1Cl] | 792 |
| 362 | 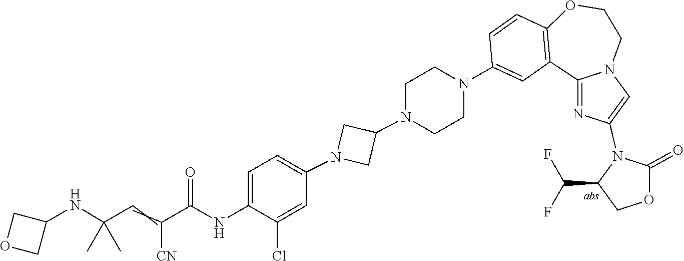 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1Cl)NC1COC1] | 778 |
| 363 | 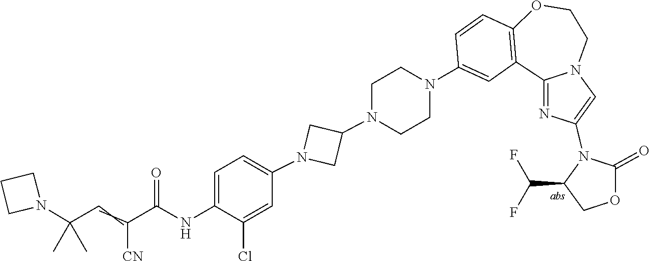 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1Cl)N1CCC1] | 762 |
| 364 | 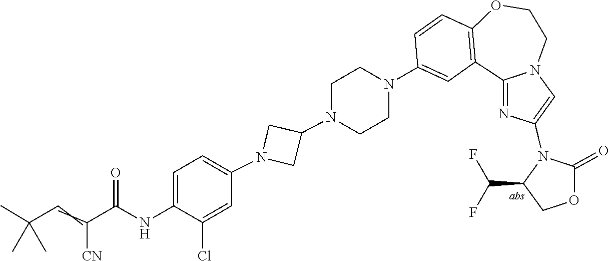 [SMILES: CC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1Cl] | 721 |
| 365 | 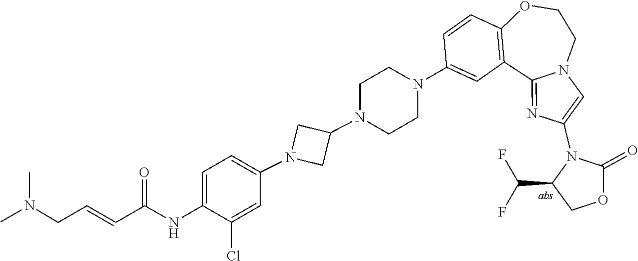 [SMILES: CN(C)C/C=C/C(=O)Nc1ccc(N2CC(N3CCN(c4ccc5c(c4)-c4nc(N6C(=O)OC[C@H]6C(F)F)cn4CCO5)CC3)C2)cc1Cl] | 697 |
| 366 | 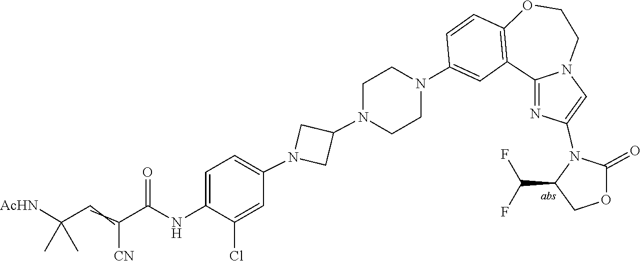 [SMILES: CC(=O)NC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1Cl] | 764 |
| 367 | 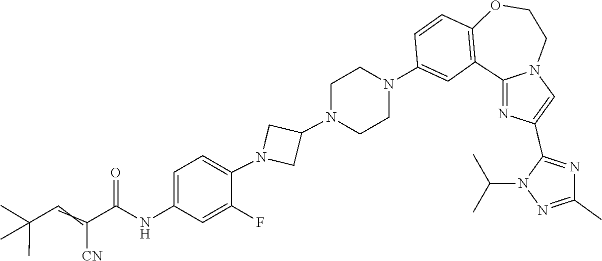 [SMILES: Cc1nc(-c2cn3c(n2)-c2cc(N4CCN(C5CN(c6ccc(NC(=O)C(C#N)=CC(C)(C)C)cc6F)C5)CC4)ccc2OCC3)n(C(C)C)n1] | 693 |
| 368 | 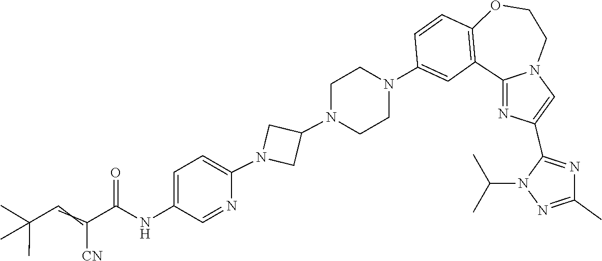 [SMILES: Cc1nc(-c2cn3c(n2)-c2cc(N4CCN(C5CN(c6ccc(NC(=O)C(C#N)=CC(C)(C)C)cn6)C5)CC4)ccc2OCC3)n(C(C)C)n1] | 676 |
| 369 | 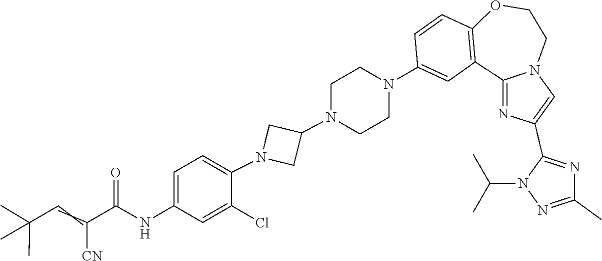 [SMILES: Cc1nc(-c2cn3c(n2)-c2cc(N4CCN(C5CN(c6ccc(NC(=O)C(C#N)=CC(C)(C)C)cc6Cl)C5)CC4)ccc2OCC3)n(C(C)C)n1] | 709 |
| 370 | 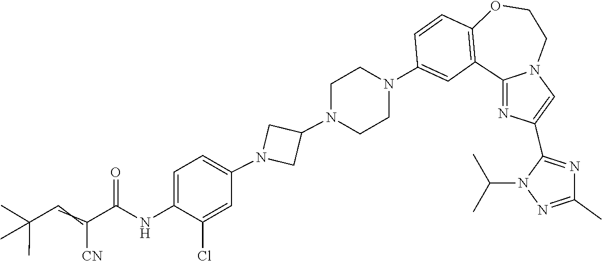 [SMILES: Cc1nc(-c2cn3c(n2)-c2cc(N4CCN(C5CN(c6ccc(NC(=O)C(C#N)=CC(C)(C)C)c(Cl)c6)C5)CC4)ccc2OCC3)n(C(C)C)n1] | 709 |
| 371 | 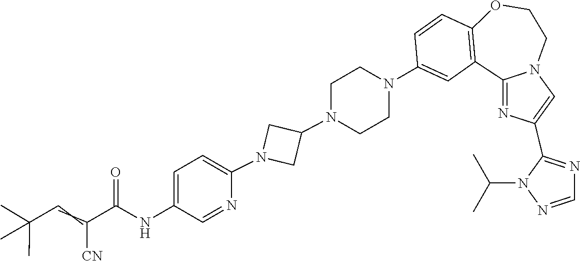 [SMILES: CC(C)n1ncnc1-c1cn2c(n1)-c1cc(N3CCN(C4CN(c5ccc(NC(=O)C(C#N)=CC(C)(C)C)cn5)C4)CC3)ccc1OCC2] | 662 |
| 372 | 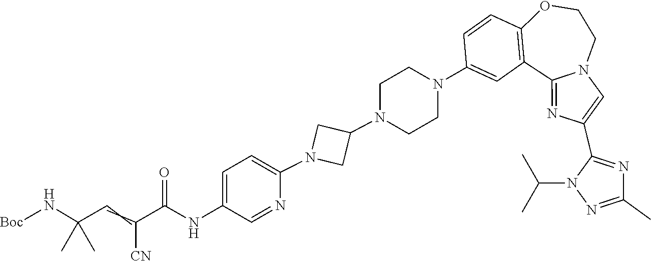 [SMILES: Cc1nc(-c2cn3c(n2)-c2cc(N4CCN(C5CN(c6ccc(NC(=O)C(C#N)=CC(C)(C)NC(=O)OC(C)(C)C)cn6)C5)CC4)ccc2OCC3)n(C(C)C)n1] | 777 |
| 373 | 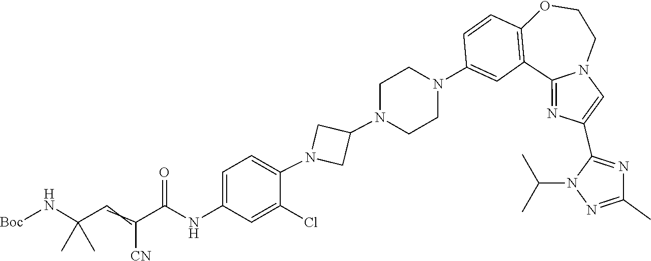 [SMILES: Cc1nc(-c2cn3c(n2)-c2cc(N4CCN(C5CN(c6ccc(NC(=O)C(C#N)=CC(C)(C)NC(=O)OC(C)(C)C)cc6Cl)C5)CC4)ccc2OCC3)n(C(C)C)n1] | 810 |
| 374 | 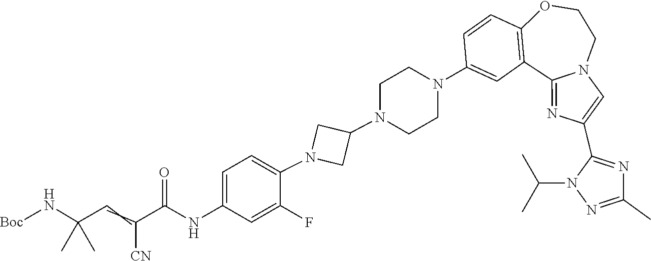 [SMILES: Cc1nc(-c2cn3c(n2)-c2cc(N4CCN(C5CN(c6ccc(NC(=O)C(C#N)=CC(C)(C)NC(=O)OC(C)(C)C)cc6F)C5)CC4)ccc2OCC3)n(C(C)C)n1] | 794 |
| 375 | 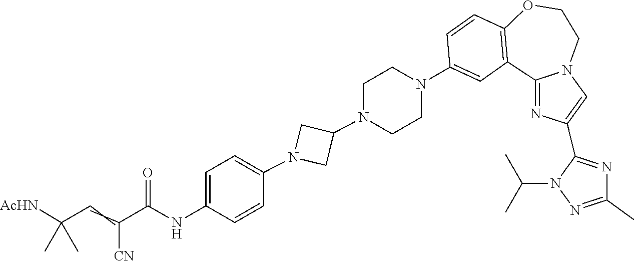 [SMILES: CC(=O)NC(C)(C)C=C(C#N)C(=O)Nc1ccc(N2CC(N3CCN(c4ccc5c(c4)-c4nc(-c6nc(C)nn6C(C)C)cn4CCO5)CC3)C2)cc1] | 718 |
| 376 | 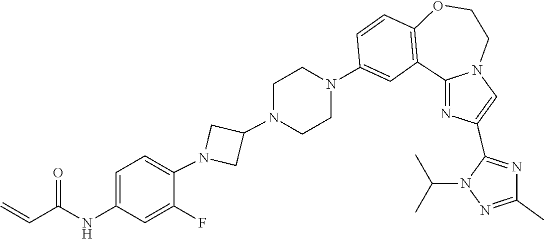 [SMILES: C=CC(=O)Nc1ccc(N2CC(N3CCN(c4ccc5c(c4)-c4nc(-c6nc(C)nn6C(C)C)cn4CCO5)CC3)C2)c(F)c1] | 612 |
| 377 | 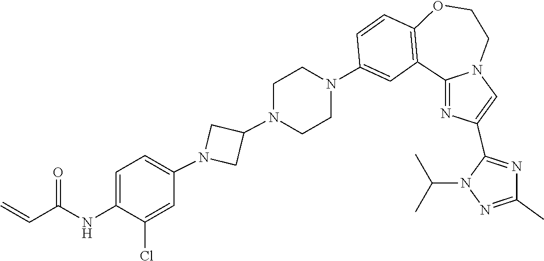 [SMILES: C=CC(=O)Nc1ccc(N2CC(N3CCN(c4ccc5c(c4)-c4nc(-c6nc(C)nn6C(C)C)cn4CCO5)CC3)C2)cc1Cl] | 628 |
| 378 | 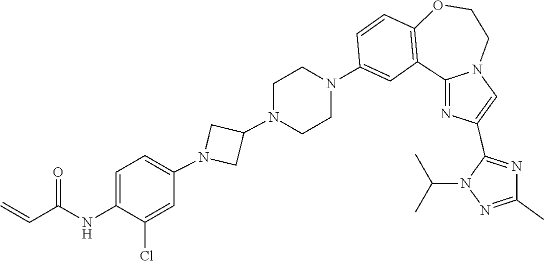 [SMILES: C=CC(=O)Nc1ccc(N2CC(N3CCN(c4ccc5c(c4)-c4nc(-c6nc(C)nn6C(C)C)cn4CCO5)CC3)C2)cc1Cl] | 628 |
| 379 | 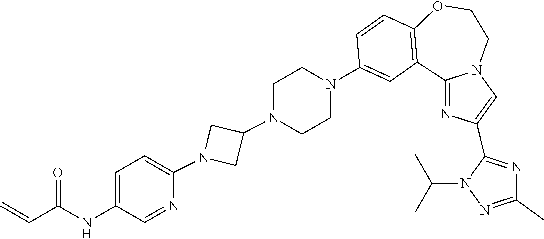 [SMILES: C=CC(=O)Nc1ccc(N2CC(N3CCN(c4ccc5c(c4)-c4nc(-c6nc(C)nn6C(C)C)cn4CCO5)CC3)C2)nc1] | 595 |
| 380 | 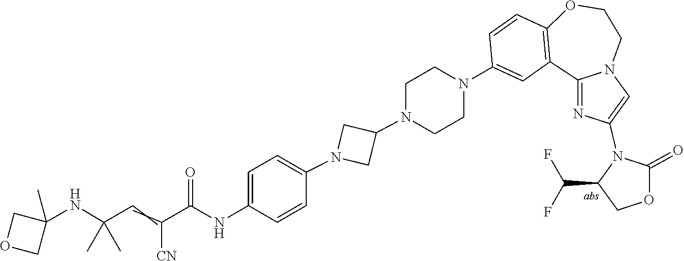 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)NC1(C)COC1] | 758 |
| 381 | 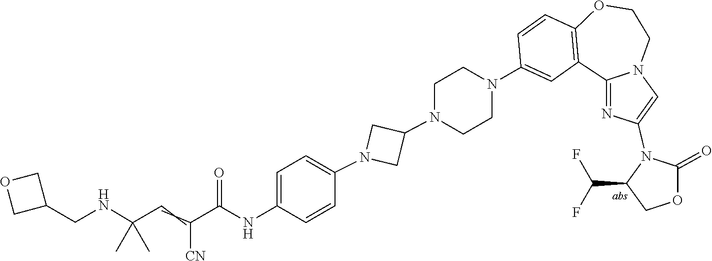 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)NCC1COC1] | 758 |
| 382 | 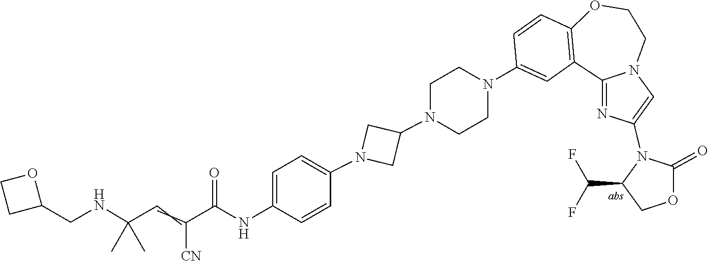 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)NCC1CCO1] | 758 |
| 383 | 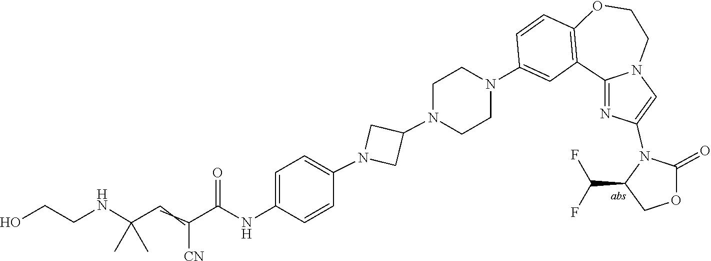 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)NCCO] | 732 |
| 384 | 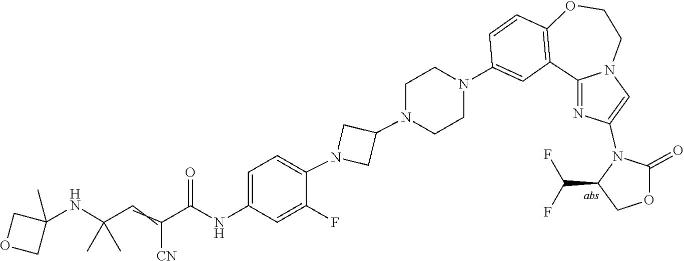 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1)NC1(C)COC1] | 776 |
| 385 | 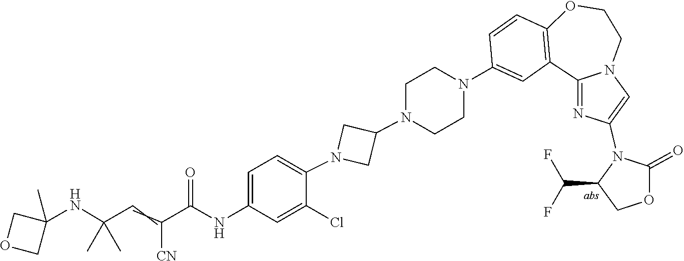 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)c(Cl)c1)NC1(C)COC1] | 792 |
| 386 | 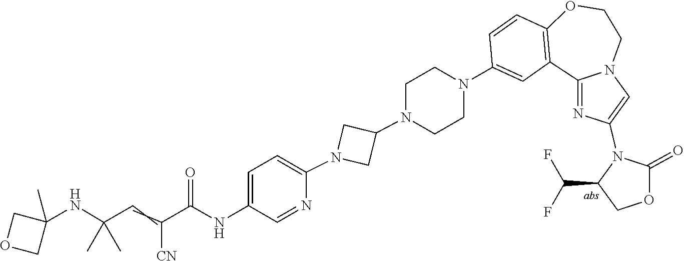 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)nc1)NC1(C)COC1] | 759 |
| 387 | 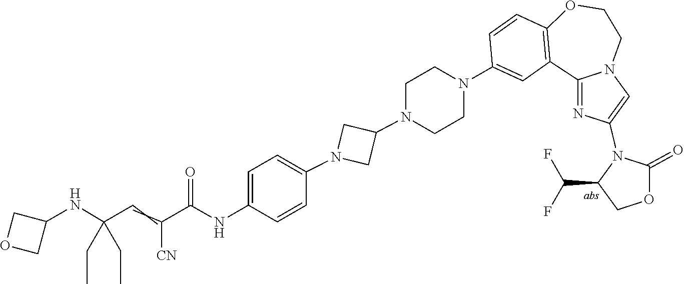 [SMILES: CCC(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)(CC)NC1COC1] | 772 |
| 388 | 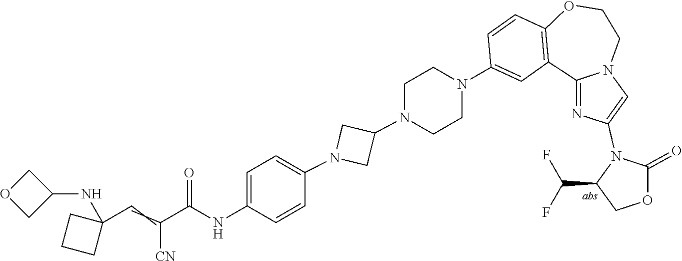 [SMILES: N#CC(=CC1(NC2COC2)CCC1)C(=O)Nc1ccc(N2CC(N3CCN(c4ccc5c(c4)-c4nc(N6C(=O)OC[C@H]6C(F)F)cn4CCO5)CC3)C2)cc1] | 756 |
| 389 | 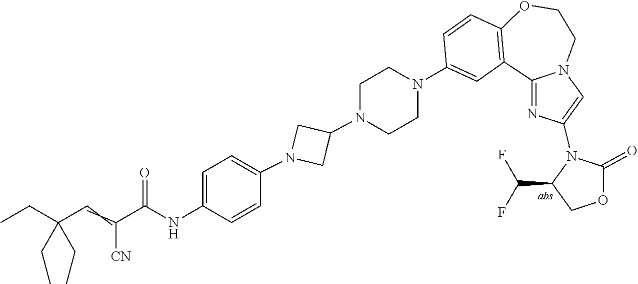 [SMILES: CCC(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)(CC)CC] | 729 |
| 390 | 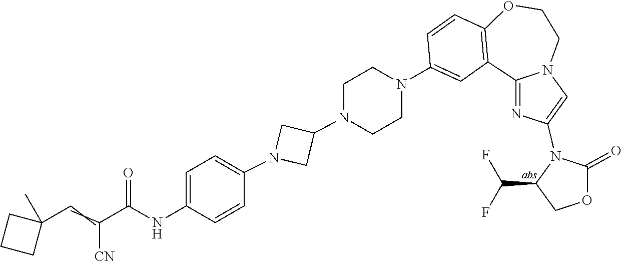 [SMILES: CC1(C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CCC1] | 699 |
| 391 | 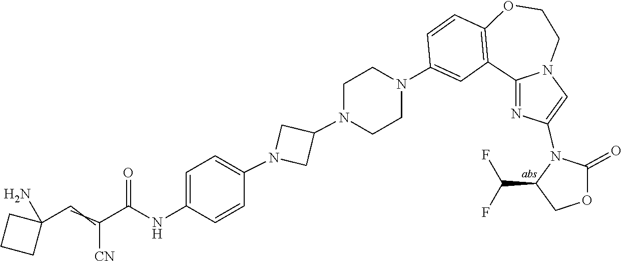 [SMILES: N#CC(=CC1(N)CCC1)C(=O)Nc1ccc(N2CC(N3CCN(c4ccc5c(c4)-c4nc(N6C(=O)OC[C@H]6C(F)F)cn4CCO5)CC3)C2)cc1] | 700 |
| 392 | 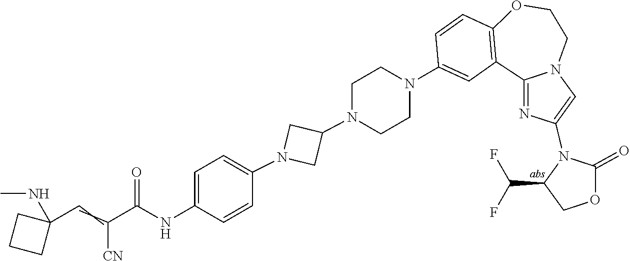 [SMILES: CNC1(C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CCC1] | 714 |
| 393 | 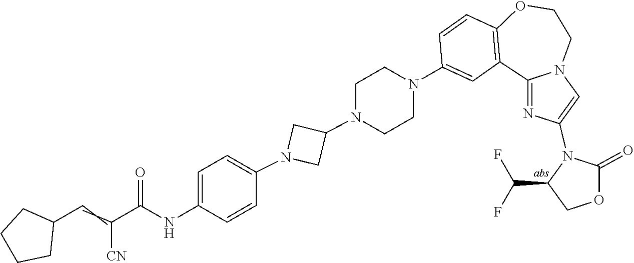 [SMILES: N#CC(=CC1CCCC1)C(=O)Nc1ccc(N2CC(N3CCN(c4ccc5c(c4)-c4nc(N6C(=O)OC[C@H]6C(F)F)cn4CCO5)CC3)C2)cc1] | 699 |
| 394 | 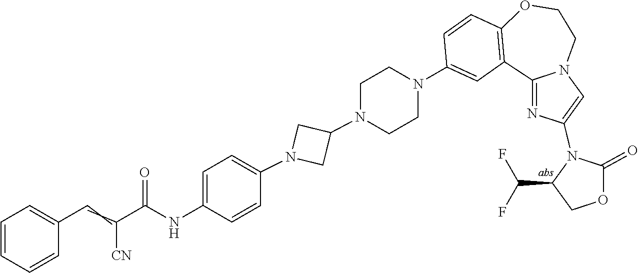 [SMILES: N#CC(=Cc1ccccc1)C(=O)Nc1ccc(N2CC(N3CCN(c4ccc5c(c4)-c4nc(N6C(=O)OC[C@H]6C(F)F)cn4CCO5)CC3)C2)cc1] | 707 |
| 395 | 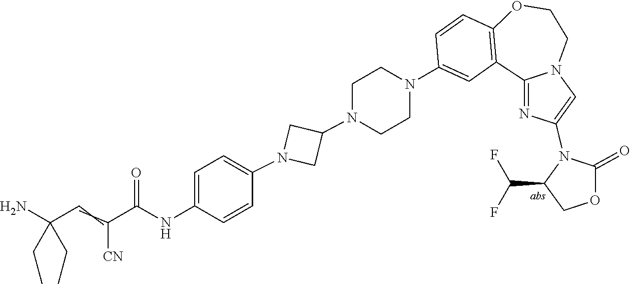 [SMILES: CCC(N)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)CC] | 716 |

TABLE 2
Nuclear magnetic resonance data of some of the compounds of the present disclosure

| Compound | Compound structure | ¹H NMR |
|---|---|---|
| 97 | 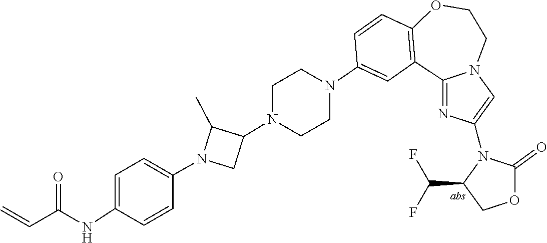 [SMILES: C=CC(=O)Nc1ccc(N2CC(N3CCN(c4ccc5c(c4)-c4nc(N6C(=O)OC[C@H]6C(F)F)cn4CCO5)CC3)C2C)cc1] | ¹H NMR (400 MHz, DMSO-d₆) δ 9.84 (s, 1H), 7.78 (d, J = 2.8 Hz, 1H), 7.46 (d, J = 8.7 Hz, 2H), 7.35 (s, 1H), 6.99-6.90 (m, 2H), 6.84- 6.52 (m, 1H), 6.38 (m, 3H), 6.18 (dd, J = 16.9, 2.2 Hz, 1H), 5.67 (dd, J = 10.1, 2.2 Hz, 1H), 5.10-4.95 (m, 1H), 4.65-4.54 (m, 2H), 4.40 (d, J = 5.5 Hz, 2H), 4.37 (dd, J = 6.2, 2.9 Hz, 2H), 4.32-4.23 (m, 1H), 3.76-3.61 (m, 2H), 3.27-3.20 (m, 1H), 3.13 (s, 4H), 2.52 (m, 4H), 1.27 (d, J = 6.3 Hz, 3H). |
| 98 | 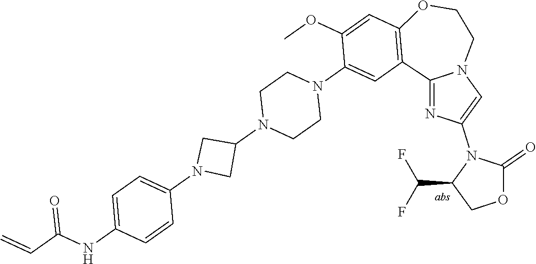 [SMILES: C=CC(=O)Nc1ccc(N2CC(N3CCN(c4cc5c(cc4OC)OCCn4cc(N6C(=O)OC[C@H]6C(F)F)nc4-5)CC3)C2)cc1] | ¹H NMR (400 MHz, DMSO-d₆) δ 9.81 (s, 1H), 8.16 (s, 1H) (HCOOH), 7.72 (s, 1H), 7.47-7.40 (m, 2H), 7.22 (s, 1H), 6.75-6.49 (m, 2H), 6.41-6.29 (m, 3H), 6.15 (dd, J = 17.0, 2.1 Hz, 1H), 5.64 (dd, J = 10.1, 2.1 Hz, 1H), 4.96 (m, 1H), 4.61-4.47 (m, 2H), 4.35 (s, 4H), 3.87 (t, J = 7.0 Hz, 2H), 3.75 (s, 3H), 3.54 (m, 2H), 3.28 (m, 1H), 3.11-2.93 (m, 4H), 2.53 (m, 4H). |
| 101 | 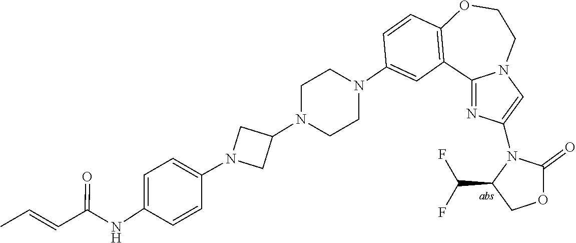 [SMILES: C/C=C/C(=O)Nc1ccc(N2CC(N3CCN(c4ccc5c(c4)-c4nc(N6C(=O)OC[C@H]6C(F)F)cn4CCO5)CC3)C2)cc1] | ¹H NMR (400 MHz, DMSO-d₆) δ 9.67 (s, 1H), 7.77 (d, J = 2.9 Hz, 1H), 7.48-7.42 (m, 2H), 7.34 (s, 1H), 6.99-6.89 (m, 2H), 6.82- 6.52 (m, 2H), 6.43-6.37 (m, 2H), 6.11-6.03 (m, 1H), 5.08-4.95 (m, 1H), 4.65-4.53 (m, 2H), 4.42-4.38 (m, 2H), 4.38-4.35 (m, 2H), 3.91 (t, J = 7.0 Hz, 2H), 3.58 (dd, J = 7.4, 5.4 Hz, 2H), 3.31 (m, 1H), 3.11 (d, J = 6.4 Hz, 4H), 2.53-2.51 (m, 4H), 1.84 (dd, J = 6.9, 1.7 Hz, 3H). |
| 102 | 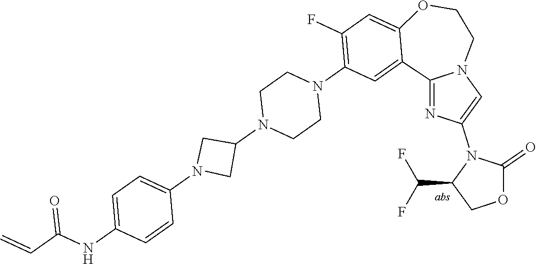 [SMILES: C=CC(=O)Nc1ccc(N2CC(N3CCN(c4cc5c(cc4F)OCCn4cc(N6C(=O)OC[C@H]6C(F)F)nc4-5)CC3)C2)cc1] | ¹H NMR (400 MHz, DMSO-d₆) δ 9.82 (s, 1H), 7.87 (d, J = 10.0 Hz, 1H), 7.47-7.41 (m, 2H), 7.30 (s, 1H), 6.85 (d, J = 12.9 Hz, 1H), 6.77-6.50 (m, 1H), 6.42-6.30 (m, 3H), 6.15 (dd, J = 17.0, 2.2 Hz, 1H), 5.64 (dd, J = 10.1, 2.2 Hz, 1H), 5.03-4.91 (m, 1H), 4.62-4.48 (m, 2H), 4.38 (s, 4H), 3.88 (t, J = 7.0 Hz, 2H), 3.60-3.50 (m, 2H), 3.06-2.89 (m, 5H), 2.53 (m, 4H). |
| 103 | 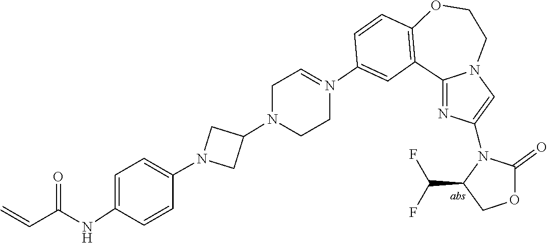 | ¹H NMR (400 MHz, DMSO-d₆) δ 9.85 (s, 1H), 8.31 (d, J = 2.5 Hz, 1H), 7.50-7.46 (m, 2H), 7.37 (d, J = 5.2 Hz, 2H), 6.99 (d, J = 8.6 Hz, 1H), 6.77-6.53 (m, 1H), 6.44-6.35 (m, 3H), 6.18 (dd, J = 17.0, 2.2 Hz, 1H), 6.13 (d, J = 3.7 Hz, 1H), 5.67 (dd, J = 10.1, 2.2 Hz, 1H), 5.08-4.99 (m, 1H), 4.64-4.56 (m, 2H), 4.44 (s, 4H), 3.95 (t, J = 7.0 Hz, 2H), 3.61 (t, J = 6.5 Hz, 2H), 3.39 (m, 1H), 3.08 (m, 2H), 2.61 (m, 4H). |
| 104 | 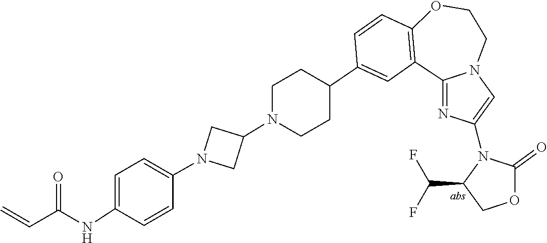 [SMILES: C=CC(=O)Nc1ccc(N2CC(N3CCC(c4ccc5c(c4)-c4nc(N6C(=O)OC[C@H]6C(F)F)cn4CCO5)CC3)C2)cc1] | ¹H NMR (400 MHz, DMSO-d₆) δ 9.82 (s, 1H), 8.10 (d, J = 2.3 Hz, 1H), 7.47-7.41 (m, 2H), 7.31 (s, 1H), 7.13 (dd, J = 8.5, 2.3 Hz, 1H), 6.91 (d, J = 8.4 Hz, 1H), 6.76-6.51 (m, 1H), 6.43-6.29 (m, 3H), 6.15 (dd, J = 16.9, 2.2 Hz, 1H), 5.64 (dd, J = 10.1, 2.2 Hz, 1H), 5.07- 4.94 (m, 1H), 4.63-4.48 (m, 2H), 4.37 (s, 4H), 3.88 (t, J = 7.0 Hz, 2H), 3.50 (dd, J = 7.4, 5.6 Hz, 2H), 3.21 (m, 1H), 2.87 (d, J = 10.7 Hz, 2H), 2.56-2.48 (m, 1H), 1.97-1.87 (m, 2H), 1.77-1.69 (m, 2H), 1.60 (m, 2H). |
| 105 | 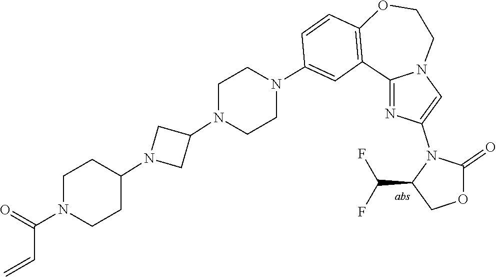 [SMILES: C=CC(=O)N1CCC(N2CC(N3CCN(c4ccc5c(c4)-c4nc(N6C(=O)OC[C@H]6C(F)F)cn4CCO5)CC3)C2)CC1] | ¹H NMR (400 MHz, DMSO-d₆) δ 7.72 (d, J = 2.8 Hz, 1H), 7.30 (s, 1H), 6.95-6.84 (m, 2H), 6.79-6.72 (m, 1H), 6.72-6.48 (m, 1H), 6.02 (dd, J = 16.7, 2.4 Hz, 1H), 5.60 (dd, J = 10.5, 2.4 Hz, 1H), 5.04- 4.91 (m, 1H), 4.58-4.53 (m, 2H), 4.40-4.29 (m, 4H), 3.95 (m, 1H), 3.80 (m, 1H), 3.13 (m, 1H), 3.04 (m, 4H), 2.86 (m, 4H), 2.38 (m, 4H), 2.25 (s, 1H), 1.59 (m, 2H), 1.03 (m, 2H). |
| 107 | 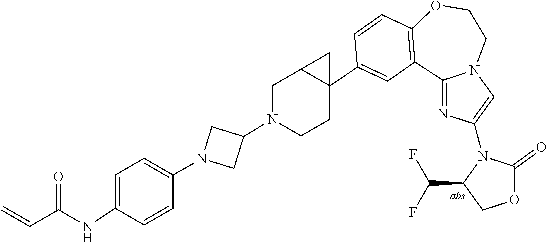 [SMILES: C=CC(=O)Nc1ccc(N2CC(N3CCC4(c5ccc6c(c5)-c5nc(N7C(=O)OC[C@H]7C(F)F)cn5CCO6)CC4C3)C2)cc1] | ¹H NMR (400 MHz, DMSO-d₆) δ 9.81 (s, 1H), 8.09 (t, J = 2.3 Hz, 1H), 7.43 (d, J = 8.5 Hz, 2H), 7.30 (s, 1H), 7.12 (d, J = 8.9 Hz, 1H), 6.90 (d, J = 8.4 Hz, 1H), 6.60 (t, J = 55.7 Hz, 1H), 6.35 (dd, J = 16.7, 9.7 Hz, 2H), 6.24-5.99 (m, 2H), 5.74-5.72 (m, 1H), 5.00 (m, 1H), 4.64-4.46 (m, 2H), 4.37 (s, 4H), 3.87 (m, 2H), 3.55-3.39 (m, 2H), 3.19 (m, 1H), 2.80-2.53 (m, 2H), 2.25-1.89 (m, 5H), 1.20 (s, 5H), 0.81 (t, J = 6.0 Hz, 2H). |
| 111 | 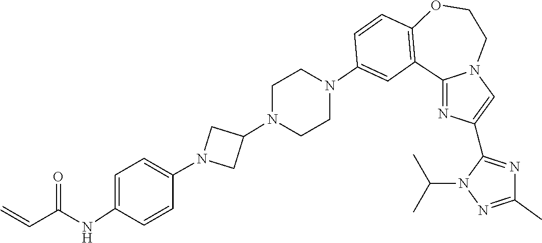 [SMILES: C=CC(=O)Nc1ccc(N2CC(N3CCN(c4ccc5c(c4)-c4nc(-c6nc(C)nn6C(C)C)cn4CCO5)CC3)C2)cc1] | ¹H NMR (400 MHz, DMSO-d₆) δ 9.90 (s, 1H), 7.91 (d, J = 3.0 Hz, 1H), 7.85 (s, 1H), 7.51-7.44 (m, 2H), 7.02 (dd, J = 9.0, 3.0 Hz, 1H), 6.94 (d, J = 8.9 Hz, 1H), 6.40 (dd, J = 17.0, 9.6 Hz, 3H), 6.18 (dd, J = 17.0, 2.2 Hz, 1H), 5.71-5.63 (m, 2H), 4.48 (dd, J = 5.5, 2.8 Hz, 2H), 4.43-4.39 (m, 2H), 3.92 (t, J = 7.0 Hz, 2H), 3.60 (dd, J = 7.4, 5.5 Hz, 2H), 3.25 (s, 1H), 3.13 (t, J = 4.8 Hz, 4H), 2.53 (d, J = 4.3 Hz, 4H), 2.25 (s, 3H), 1.47 (d, J = 6.6 Hz, 6H). |
| 121 | 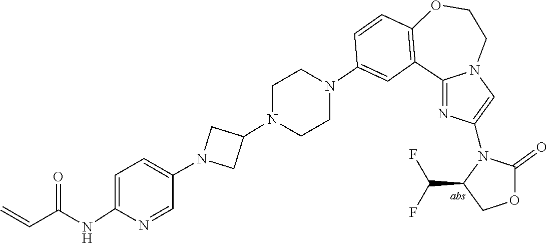 [SMILES: C=CC(=O)Nc1ccc(N2CC(N3CCN(c4ccc5c(c4)-c4nc(N6C(=O)OC[C@H]6C(F)F)cn4CCO5)CC3)C2)cn1] | ¹H NMR (400 MHz, DMSO-d₆) δ 10.39 (s, 1H), 8.02 (d, J = 8.8 Hz, 1H), 7.77 (d, J = 2.8 Hz, 1H), 7.60 (d, J = 2.9 Hz, 1H), 7.34 (s, 1H), 7.00-6.48 (m, 5H), 6.24 (dd, J = 17.0, 2.1 Hz, 1H), 5.77-5.65 (m, 1H), 5.07-4.95 (m, 1H), 4.65-4.54 (m, 2H), 4.40-4.37 (m, 4H), 3.99 (t, J = 7.1 Hz, 2H), 3.69 (t, J = 6.6 Hz, 2H), 3.39-3.37 (m, 1H), 3.12 (s, 4H), 2.56-2.52 (m, 4H). |
| 122 | 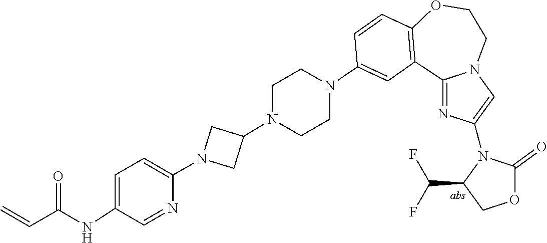 [SMILES: C=CC(=O)Nc1ccc(N2CC(N3CCN(c4ccc5c(c4)-c4nc(N6C(=O)OC[C@H]6C(F)F)cn4CCO5)CC3)C2)nc1] | ¹H NMR (400 MHz, DMSO-d₆) δ 9.93 (s, 1H), 8.27 (dd, J = 2.5, 0.7 Hz, 1H), 7.79 (dd, J = 8.9, 2.6 Hz, 1H), 7.73 (d, J = 2.9 Hz, 1H), 7.30 (s, 1H), 6.96-6.84 (m, 2H), 6.80-6.46 (m, 1H), 6.40-6.29 (m, 2H), 6.17 (dd, J = 17.0, 2.1 Hz, 1H), 5.68 (dd, J = 10.1, 2.1 Hz, 1H), 5.04- 4.92 (m, 1H), 4.62-4.49 (m, 2H), 4.38-4.31 (m, 4H), 4.00-3.92 (m, 2H), 3.73 (dd, J = 8.2, 5.2 Hz, 2H), 3.28 (m, 1H), 3.08-3.06 (m, 4H), 2.49 (s, 4H). |
| 123 | 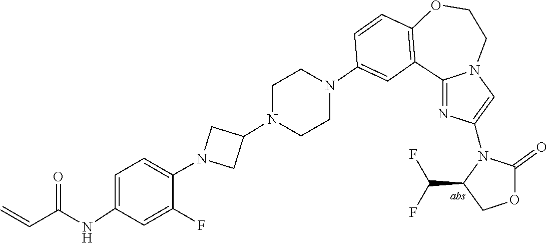 [SMILES: C=CC(=O)Nc1ccc(N2CC(N3CCN(c4ccc5c(c4)-c4nc(N6C(=O)OC[C@H]6C(F)F)cn4CCO5)CC3)C2)c(F)c1] | ¹H NMR (400 MHz, DMSO-d₆) δ 10.05 (s, 1H), 7.77 (d, J = 2.9 Hz, 1H), 7.56 (dd, J = 14.8, 2.3 Hz, 1H), 7.34 (s, 1H), 7.20 (dd, J = 8.5, 2.2 Hz, 1H), 6.98-6.90 (m, 2H), 6.67 (d, J = 2.9 Hz, 1H), 6.58-6.52 (m, 1H), 6.37 (dd, J = 16.9, 10.0 Hz, 1H), 6.21 (dd, J = 17.0, 2.2 Hz, 1H), 5.71 (dd, J = 10.0, 2.1 Hz, 1H), 5.08-4.95 (m, 1H), 4.62-4.56 (m, 2H), 4.42-4.36 (m, 4H), 4.02-3.97 (m, 2H), 3.69 (t, J = 6.6 Hz, 2H), 3.32 (m, 1H), 3.14-3.08 (m, 4H), 2.52 (m, 4H). |
| 124 | 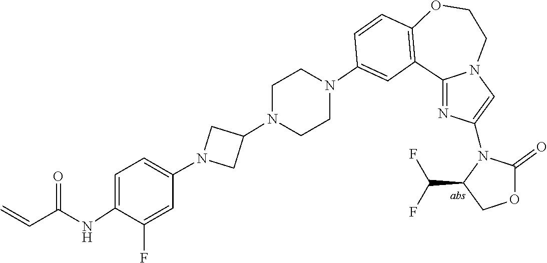 [SMILES: C=CC(=O)Nc1ccc(N2CC(N3CCN(c4ccc5c(c4)-c4nc(N6C(=O)OC[C@H]6C(F)F)cn4CCO5)CC3)C2)cc1F] | ¹H NMR (400 MHz, DMSO-d₆) δ 9.59 (s, 1H), 7.77 (d, J = 2.9 Hz, 1H), 7.54 (t, J = 8.7 Hz, 1H), 7.34 (s, 1H), 6.99-6.89 (m, 2H), 6.83- 6.44 (m, 2H), 6.32 (dd, J = 12.8, 2.5 Hz, 1H), 6.26-6.16 (m, 2H), 5.70 (dd, J = 10.2, 2.1 Hz, 1H), 5.08-4.95 (m, 1H), 4.67-4.52 (m, 2H), 4.42-4.34 (m, 4H), 3.94 (t, J = 7.2 Hz, 2H), 3.65 (dd, J = 7.7, 5.3 Hz, 2H), 3.32 (m, 1H), 3.11 (d, J = 4.1 Hz, 4H), 2.57-2.51 (m, 4H). |
| 129 | 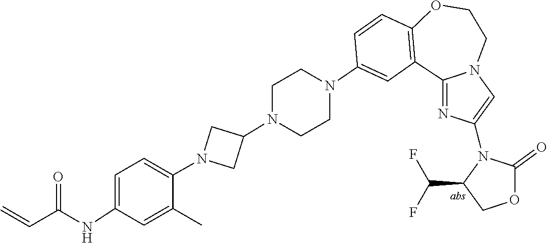 [SMILES: C=CC(=O)Nc1ccc(N2CC(N3CCN(c4ccc5c(c4)-c4nc(N6C(=O)OC[C@H]6C(F)F)cn4CCO5)CC3)C2)c(C)c1] | ¹H NMR (400 MHz, DMSO-d₆) δ 9.85 (s, 1H), 7.77 (d, J = 2.9 Hz, 1H), 7.38 (dd, J = 8.6, 2.5 Hz, 1H), 7.34 (s, 1H), 7.30 (d, J = 2.4 Hz, 1H), 6.96 (dd, J = 9.0, 2.9 Hz, 1H), 6.91 (d, J = 8.9 Hz, 1H), 6.85- 6.52 (m, 1H), 6.48-6.34 (m, 2H), 6.19 (dd, J = 16.9, 2.2 Hz, 1H), 5.68 (dd, J = 10.1, 2.2 Hz, 1H), 5.10-4.94 (m, 1H), 4.62-4.57 (m, 2H), 4.41-4.35 (m, 4H), 3.96 (t, J = 6.9 Hz, 2H), 3.62 (t, J = 6.6 Hz, 2H), 3.21 (t, J = 6.3 Hz, 1H), 3.13-3.06 (m, 4H), 2.54 (m, 4H), 2.14 (s, 3H). |
| 130 | 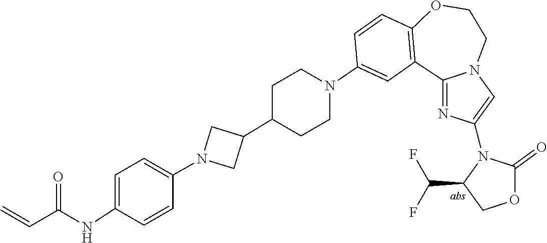 [SMILES: C=CC(=O)Nc1ccc(N2CC(C3CCN(c4ccc5c(c4)-c4nc(N6C(=O)OC[C@H]6C(F)F)cn4CCO5)CC3)C2)cc1] | ¹H NMR (400 MHz, DMSO-d₆) δ 9.80 (s, 1H), 7.74 (d, J = 3.0 Hz, 1H), 7.47-7.39 (m, 2H), 7.30 (s, 1H), 6.91 (dd, J = 9.0, 2.9 Hz, 1H), 6.86 (d, J = 8.9 Hz, 1H), 6.80-6.46 (m, 1H), 6.40-6.29 (m, 3H), 6.15 (dd, J = 17.0, 2.2 Hz, 1H), 5.63 (dd, J = 10.1, 2.2 Hz, 1H), 5.05- 4.91 (m, 1H), 4.62-4.50 (m, 2H), 4.40-4.29 (m, 4H), 3.83 (t, J = 7.5 Hz, 2H), 3.56 (s, 2H), 3.51-3.41 (m, 2H), 2.61-2.58 (m, 2H), 2.45 (m, 1H), 1.73 (d, J = 12.5 Hz, 2H), 1.59-1.49 (m, 1H), 1.23 (m, 2H). |
| 131 | 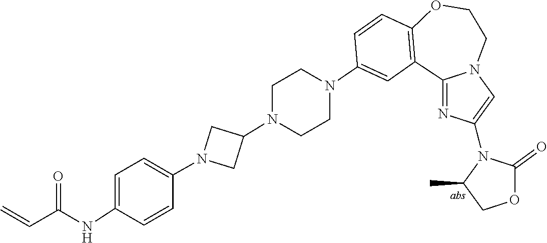 [SMILES: C=CC(=O)Nc1ccc(N2CC(N3CCN(c4ccc5c(c4)-c4nc(N6C(=O)OC[C@H]6C)cn4CCO5)CC3)C2)cc1] | ¹H NMR (400 MHz, DMSO-d₆) δ 9.83 (s, 1H), 8.26 (s, 1H), 7.75 (d, J = 2.8 Hz, 1H), 7.48-7.40 (m, 2H), 7.26 (s, 1H), 6.95-6.83 (m, 2H), 6.42-6.29 (m, 3H), 6.15 (dd, J = 16.9, 2.2 Hz, 1H), 5.63 (dd, J = 10.1, 2.1 Hz, 1H), 4.59-4.50 (m, 2H), 4.34-4.32 (m, 4H), 4.05- 3.99 (m, 1H), 3.88 (t, J = 7.0 Hz, 2H), 3.58-3.54 (m, 4H), 3.26 (t, J = 6.1 Hz, 2H), 3.05 (t, J = 4.9 Hz, 4H), 1.37 (d, J = 5.8 Hz, 3H). |
| 132 | 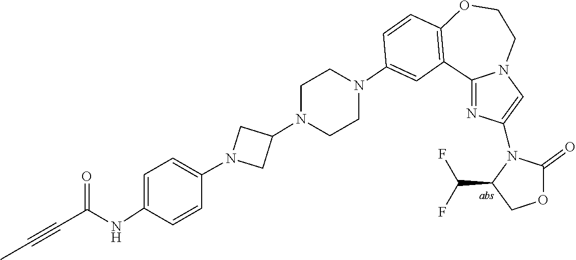 [SMILES: CC#CC(=O)Nc1ccc(N2CC(N3CCN(c4ccc5c(c4)-c4nc(N6C(=O)OC[C@H]6C(F)F)cn4CCO5)CC3)C2)cc1] | ¹H NMR (400 MHz, DMSO-d₆) δ 10.22 (s, 1H), 7.73 (d, J = 2.7 Hz, 1H), 7.37-7.33 (m, 2H), 7.30 (s, 1H), 6.94-6.86 (m, 2H), 6.63 (d, J = 2.6 Hz, 1H), 6.37-6.33 (m, 2H), 5.03-4.91 (m, 1H), 4.58-4.51 (m, 2H), 4.34 (q, J = 5.6 Hz, 4H), 3.87 (t, J = 7.0 Hz, 2H), 3.55 (t, J = 6.3 Hz, 2H), 3.27 (m, 1H), 3.07 (s, 4H), 1.97 (s, 3H). |
| 133 | 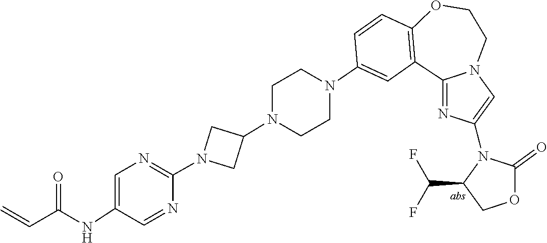 [SMILES: C=CC(=O)Nc1cnc(N2CC(N3CCN(c4ccc5c(c4)-c4nc(N6C(=O)OC[C@H]6C(F)F)cn4CCO5)CC3)C2)nc1] | ¹H NMR (400 MHz, DMSO-d₆) δ 10.05 (s, 1H), 8.55 (s, 2H), 7.73 (d, J = 2.9 Hz, 1H), 7.30 (s, 1H), 6.96-6.84 (m, 2H), 6.79-6.46 (m, 1H), 6.34 (dd, J = 17.0, 10.1 Hz, 1H), 6.20 (dd, J = 16.9, 2.1 Hz, 1H), 5.72 (dd, J = 10.0, 2.1 Hz, 1H), 5.05-4.91 (m, 1H), 4.64-4.49 (m, 2H), 4.40-4.30 (m, 4H), 4.05 (dd, J = 8.9, 7.0 Hz, 2H), 3.85 (dd, J = 9.0, 5.0 Hz, 2H), 3.29 (m, 1H), 3.07 (m, 4H), 2.50 (m, 4H). |
| 134 | 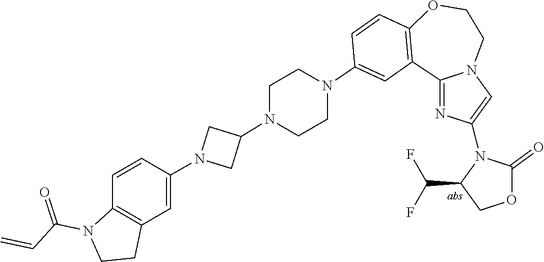 [SMILES: C=CC(=O)N1CCc2cc(N3CC(N4CCN(c5ccc6c(c5)-c5nc(N7C(=O)OC[C@H]7C(F)F)cn5CCO6)CC4)C3)ccc21] | ¹H NMR (400 MHz, DMSO-d₆) δ 7.97 (d, J = 8.6 Hz, 1H), 7.73 (d, J = 2.9 Hz, 1H), 7.30 (s, 1H), 6.99-6.84 (m, 2H), 6.82-6.44 (m, 2H), 6.41-6.29 (m, 1H), 6.27-6.14 (m, 2H), 5.70 (dd, J = 10.2, 2.4 Hz, 1H), 5.09-4.85 (m, 1H), 4.67-4.44 (m, 2H), 4.42-4.27 (m, 4H), 4.12 (t, J = 8.3 Hz, 2H), 3.88 (t, J = 7.0 Hz, 2H), 3.61-3.51 (m, 2H), 3.45 (m, 1H), 3.06 (m, 6H), 2.51 (m, 4H). |
| 135 | 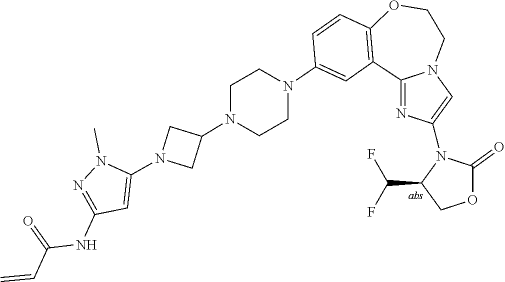 [SMILES: C=CC(=O)Nc1cc(N2CC(N3CCN(c4ccc5c(c4)-c4nc(N6C(=O)OC[C@H]6C(F)F)cn4CCO5)CC3)C2)n(C)n1] | ¹H NMR (400 MHz, DMSO-d₆) δ 10.40 (s, 1H), 7.77 (d, J = 2.9 Hz, 1H), 7.33 (s, 1H), 7.02-6.87 (m, 2H), 6.67 (dd, J = 57.1, 54.3 Hz, 1H), 6.42 (dd, J = 17.0, 10.1 Hz, 1H), 6.19 (dd, J = 17.0, 2.2 Hz, 1H), 5.92 (s, 1H), 5.66 (dd, J = 10.1, 2.2 Hz, 1H), 5.10-4.95 (m, 1H), 4.67-4.52 (m, 2H), 4.38 (m, 4H), 3.96 (t, J = 7.1 Hz, 2H), 3.70 (t, J = 6.6 Hz, 2H), 3.46 (s, 4H), 3.11 (m, 4H), 2.51 (m, 4H). |
| 136 | 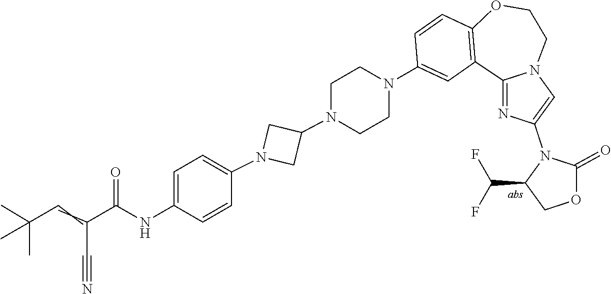 [SMILES: CC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1] | ¹H NMR (400 MHz, DMSO-d₆) δ 9.86 (s, 1H), 7.73 (d, J = 2.9 Hz, 1H), 7.42-7.28 (m, 4H), 6.96-6.84 (m, 2H), 6.77-6.49 (m, 1H), 6.43-6.34 (m, 2H), 5.04-4.92 (m, 1H), 4.63-4.49 (m, 2H), 4.39- 4.30 (m, 4H), 3.89 (t, J = 7.0 Hz, 2H), 3.61-3.54 (m, 2H), 3.29 (m, 1H), 3.07 (s, 4H), 2.51 (m, 4H), 1.23 (s, 9H). |
| 137 | 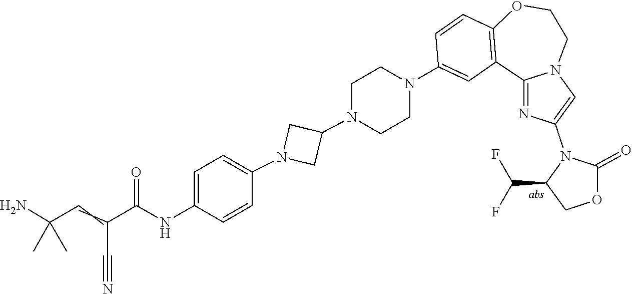 [SMILES: CC(C)(N)C=C(C#N)C(=O)Nc1ccc(N2CC(N3CCN(c4ccc5c(c4)-c4nc(N6C(=O)OC[C@H]6C(F)F)cn4CCO5)CC3)C2)cc1] | ¹H NMR (400 MHz, DMSO-d₆) δ 10.50 (s, 1H), 8.24 (s, 1H), 7.77 (d, J = 2.9 Hz, 1H), 7.53-7.46 (m, 2H), 7.34 (s, 1H), 6.99-6.90 (m, 2H), 6.83-6.52 (m, 1H), 6.48-6.41 (m, 2H), 5.07-4.95 (m, 1H), 4.62-4.56 (m, 2H), 4.42-4.39 (m, 2H), 4.38-4.35 (m, 2H), 3.94 (t, J = 7.1 Hz, 2H), 3.62 (dd, J = 7.5, 5.4 Hz, 2H), 3.33-3.27 (m, 1H), 3.14-3.09 (m, 4H), 2.53 (m, 4H), 1.44 (s, 6H). |
| 138 | 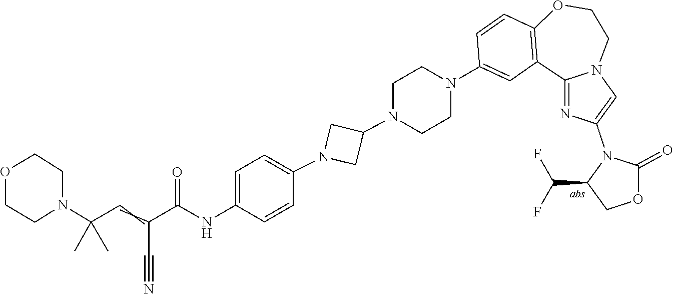 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)N1CCOCC1] | ¹H NMR (400 MHz, DMSO-d₆) δ 9.91 (s, 1H), 7.77 (d, J = 2.9 Hz, 1H), 7.40 (d, J = 8.5 Hz, 2H), 7.33 (d, J = 9.0 Hz, 2H), 7.00-6.88 (m, 2H), 6.83-6.54 (m, 1H), 6.44 (d, J = 8.5 Hz, 2H), 5.08-4.95 (m, 1H), 4.64-4.55 (m, 2H), 4.42-4.35 (m, 4H), 3.94 (s, 2H), 3.64 (m, 6H), 3.32 (m, 1H), 3.11 (s, 4H), 2.93 (m, 2H), 2.55-2.52 (m, 6H), 1.27 (s, 6H). |
| 139 | 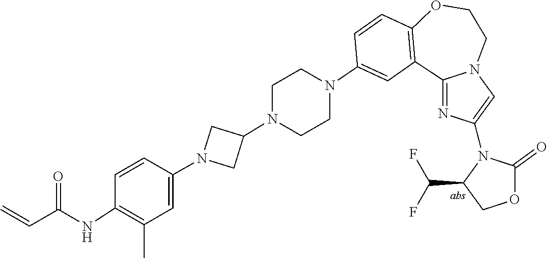 [SMILES: C=CC(=O)Nc1ccc(N2CC(N3CCN(c4ccc5c(c4)-c4nc(N6C(=O)OC[C@H]6C(F)F)cn4CCO5)CC3)C2)cc1C] | ¹H NMR (400 MHz, DMSO-d₆) δ 9.27 (s, 1H), 7.77 (d, J = 2.8 Hz, 1H), 7.34 (s, 1H), 7.15 (d, J = 8.4 Hz, 1H), 7.03-6.88 (m, 2H), 6.68 (t, J = 55.7 Hz, 1H), 6.46 (dd, J = 17.0, 10.2 Hz, 1H), 6.36-6.24 (m, 2H), 6.18 (dd, J = 17.0, 2.2 Hz, 1H), 5.68 (dd, J = 10.2, 2.1 Hz, 1H), 5.12-4.95 (m, 1H), 4.67-4.53 (m, 2H), 4.46-4.33 (m, 4H), 3.92 (t, J = 7.0 Hz, 2H), 3.67-3.56 (m, 2H), 3.29 (m, 1H), 3.18-3.05 (m, 4H), 2.70-2.60 (m, 4H), 2.12 (s, 3H). |
| 140 | 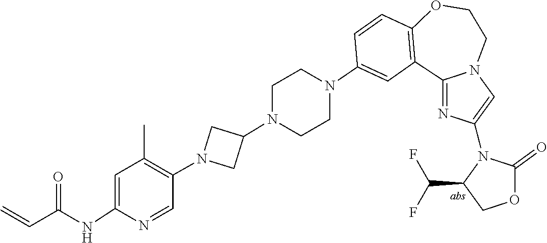 [SMILES: C=CC(=O)Nc1cc(C)c(N2CC(N3CCN(c4ccc5c(c4)-c4nc(N6C(=O)OC[C@H]6C(F)F)cn4CCO5)CC3)C2)cn1] | ¹H NMR (400 MHz, DMSO-d₆) δ 10.39 (s, 1H), 7.88 (s, 1H), 7.77 (d, J = 2.7 Hz, 1H), 7.53 (s, 1H), 7.34 (s, 1H), 6.99-6.89 (m, 2H), 6.60-6.50 (m, 1H), 6.35-6.19 (m, 1H), 5.69 (m, 1H), 5.08-4.96 (m, 1H), 4.66-4.53 (m, 2H), 4.44-4.33 (m, 4H), 4.04 (t, J = 7.0 Hz, 2H), 3.83-3.70 (m, 2H), 3.34 (m, 1H), 3.11 (m, 4H), 2.52 (m, 4H), 2.19 (s, 3H). |
| 141 | 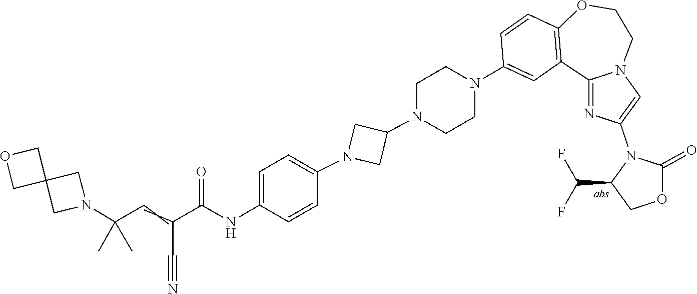 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)N1CC2(COC2)C1] | ¹H NMR (400 MHz, DMSO-d₆) δ 9.90 (s, 1H), 7.77 (d, J = 2.8 Hz, 1H), 7.40 (d, J = 8.7 Hz, 2H), 7.34 (s, 1H), 7.29 (s, 1H), 6.96 (dd, J = 9.0, 2.9 Hz, 1H), 6.91 (d, J = 8.8 Hz, 1H), 6.84-6.52 (m, 1H), 6.45- 6.41 (m, 2H), 5.08-4.95 (m, 1H), 4.64-4.55 (m, 6H), 4.42-4.39 (m, 2H), 4.38-4.35 (m, 2H), 3.93 (t, J = 7.0 Hz, 2H), 3.61 (dd, J = 7.5, 5.4 Hz, 2H), 3.36 (s, 4H), 3.29 (d, J = 2.5 Hz, 1H), 3.15-3.06 (m, 4H), 2.52 (m, 4H), 1.19 (s, 6H). |
| 142 | 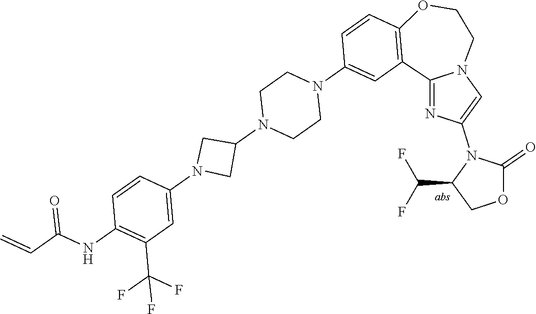 [SMILES: C=CC(=O)Nc1ccc(N2CC(N3CCN(c4ccc5c(c4)-c4nc(N6C(=O)OC[C@H]6C(F)F)cn4CCO5)CC3)C2)cc1C(F)(F)F] | ¹H NMR (400 MHz, DMSO-d₆) δ 9.44 (s, 1H), 7.73 (d, J = 2.9 Hz, 1H), 7.30 (s, 1H), 7.18 (d, J = 8.5 Hz, 1H), 6.96-6.84 (m, 2H), 6.81- 6.47 (m, 3H), 6.42 (dd, J = 17.0, 10.3 Hz, 1H), 6.14 (dd, J = 17.1, 2.1 Hz, 1H), 5.73-5.63 (m, 1H), 5.04-4.91 (m, 1H), 4.62-4.48 (m, 2H), 4.40-4.29 (m, 4H), 3.98 (t, J = 7.3 Hz, 2H), 3.69 (dd, J = 7.8, 5.2 Hz, 2H), 3.35-3.29 (m, 1H), 3.12-3.02 (m, 4H), 2.50 (d, J = 5.1 Hz, 4H). |
| 143 | 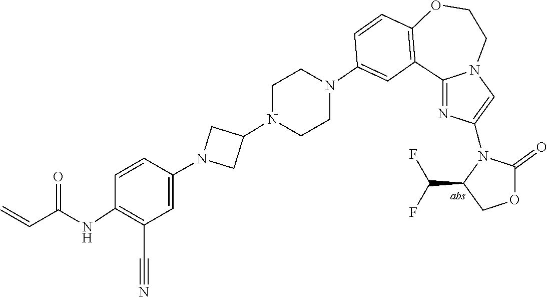 [SMILES: C=CC(=O)Nc1ccc(N2CC(N3CCN(c4ccc5c(c4)-c4nc(N6C(=O)OC[C@H]6C(F)F)cn4CCO5)CC3)C2)cc1C#N] | ¹H NMR (400 MHz, DMSO-d₆) δ 9.98 (s, 1H), 7.73 (d, J = 2.9 Hz, 1H), 7.29 (d, J = 7.5 Hz, 2H), 6.95-6.82 (m, 2H), 6.80-6.73 (m, 1H), 6.70 (dd, J = 8.8, 2.8 Hz, 1H), 6.66-6.34 (m, 2H), 6.21 (dd, J = 17.0, 2.1 Hz, 1H), 5.73 (dd, J = 10.1, 2.1 Hz, 1H), 4.98-4.96 (m, 1H), 4.67-4.45 (m, 2H), 4.39-4.29 (m, 4H), 3.94 (t, J = 7.3 Hz, 2H), 3.67 (dd, J = 7.8, 5.1 Hz, 2H), 3.30 (d, J = 6.2 Hz, 1H), 3.09- 3.07 (m, 4H), 2.48 (m, 4H). |
| 144 | 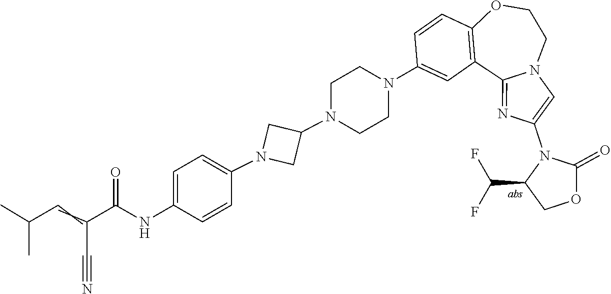 [SMILES: CC(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1] | ¹H NMR (400 MHz, DMSO-d₆) δ 9.92 (s, 1H), 7.77 (d, J = 2.9 Hz, 1H), 7.43-7.32 (m, 4H), 6.96 (dd, J = 9.0, 2.9 Hz, 1H), 6.91 (d, J = 9.0 Hz, 1H), 6.84-6.51 (m, 1H), 6.45-6.41 (m, 2H), 5.08-4.95 (m, 1H), 4.64-4.54 (m, 2H), 4.41-4.35 (m, 4H), 3.93 (t, J = 7.1 Hz, 2H), 3.61 (dd, J = 7.5, 5.4 Hz, 2H), 3.33-3.29 (m, 1H), 3.11 (dd, J = 7.0, 3.8 Hz, 4H), 2.88-2.77 (m, 1H), 2.53 (d, J = 5.9 Hz, 4H), 1.13 (d, J = 6.6 Hz, 6H). |
| 145 | 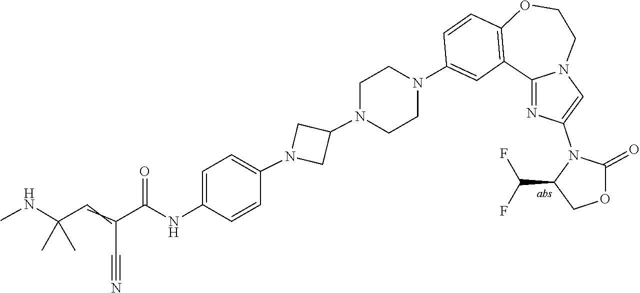 [SMILES: CNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1] | ¹H NMR (400 MHz, DMSO-d₆) δ 10.94 (s, 1H), 8.04 (s, 1H), 7.73 (d, J = 2.9 Hz, 1H), 7.46-7.42 (m, 2H), 7.30 (s, 1H), 6.93 (dd, J = 9.0, 2.9 Hz, 1H), 6.88 (d, J = 8.9 Hz, 1H), 6.78-6.49 (m, 1H), 6.44- 6.40 (m, 2H), 5.03-4.92 (m, 1H), 4.58 (t, J = 9.1 Hz, 1H), 4.52 (dd, J = 9.4, 3.8 Hz, 1H), 4.38-4.31 (m, 4H), 3.90 (t, J = 7.1 Hz, 2H), 3.58 (dd, J = 7.5, 5.4 Hz, 2H), 3.29 (m, 1H), 3.07 (s, 4H), 2.98 (s, 3H), 2.48 (m, 4H), 1.35 (s, 6H). |
| 146 | 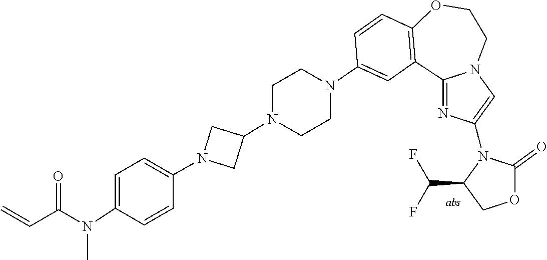 [SMILES: C=CC(=O)N(C)c1ccc(N2CC(N3CCN(c4ccc5c(c4)-c4nc(N6C(=O)OC[C@H]6C(F)F)cn4CCO5)CC3)C2)cc1] | ¹H NMR (400 MHz, DMSO-d₆) δ 7.77 (d, J = 2.8 Hz, 1H), 7.34 (s, 1H), 7.05 (d, J = 8.5 Hz, 2H), 7.00-6.89 (m, 2H), 6.86-6.53 (m, 1H), 6.47 (d, J = 8.3 Hz, 2H), 6.15-5.95 (m, 2H), 5.51 (d, J = 10.8 Hz, 1H), 5.04 (s, 1H), 4.66-4.52 (m, 2H), 4.44-4.33 (m, 4H), 3.97 (t, J = 7.2 Hz, 2H), 3.72-3.64 (m, 2H), 3.17 (s, 4H), 3.15-3.04 (m, 4H), 2.54 (m, 4H). |
| 147 | 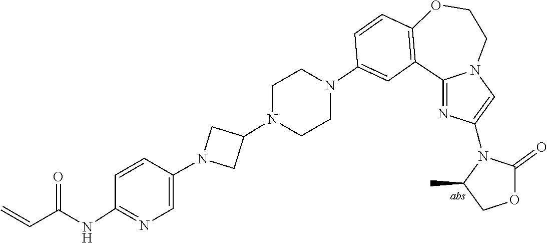 [SMILES: C=CC(=O)Nc1ccc(N2CC(N3CCN(c4ccc5c(c4)-c4nc(N6C(=O)OC[C@H]6C)cn4CCO5)CC3)C2)cn1] | ¹H NMR (400 MHz, DMSO-d₆) δ 10.40 (s, 1H), 8.02 (d, J = 8.8 Hz, 1H), 7.79 (d, J = 2.8 Hz, 1H), 7.60 (d, J = 2.9 Hz, 1H), 7.29 (s, 1H), 6.97-6.90 (m, 3H), 6.55 (dd, J = 17.0, 10.2 Hz, 1H), 6.24 (dd, J = 17.0, 2.1 Hz, 1H), 5.70 (dd, J = 10.1, 2.1 Hz, 1H), 4.60-4.53 (m, 2H), 4.37 (d, J = 3.7 Hz, 4H), 4.08-4.04 (m, 1H), 3.99 (t, J = 7.1 Hz, 2H), 3.68 (t, J = 6.6 Hz, 2H), 3.28 (s, 1H), 3.10 (s, 4H), 2.53 (s, 4H), 1.41 (d, J = 5.7 Hz, 3H). |
| 148 | 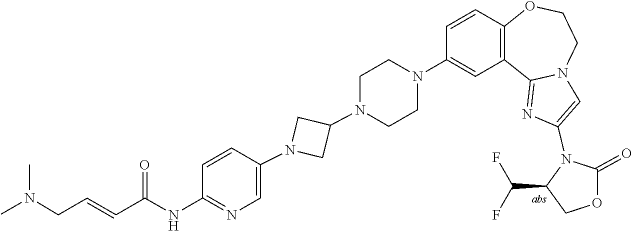 [SMILES: CN(C)C/C=C/C(=O)Nc1ccc(N2CC(N3CCN(c4ccc5c(c4)-c4nc(N6C(=O)OC[C@H]6C(F)F)cn4CCO5)CC3)C2)cn1] | ¹H NMR (400 MHz, DMSO-d₆) δ 10.28 (s, 1H), 8.00 (d, J = 8.9 Hz, 1H), 7.77 (d, J = 2.9 Hz, 1H), 7.59 (d, J = 2.9 Hz, 1H), 7.34 (s, 1H), 6.98-6.90 (m, 3H), 6.82-6.51 (m, 2H), 6.40-6.32 (m, 1H), 5.08- 4.95 (m, 1H), 4.66-4.52 (m, 2H), 4.42-4.38 (m, 2H), 4.37 (d, J = 5.5 Hz, 2H), 3.98 (t, J = 7.2 Hz, 2H), 3.68 (dd, J = 7.6, 5.4 Hz, 2H), 3.29 (s, 1H), 3.12 (d, J = 6.3 Hz, 4H), 3.02 (dd, J = 6.2, 1.6 Hz, 2H), 2.53 (s, 4H), 2.15 (s, 6H). |
| 149 | 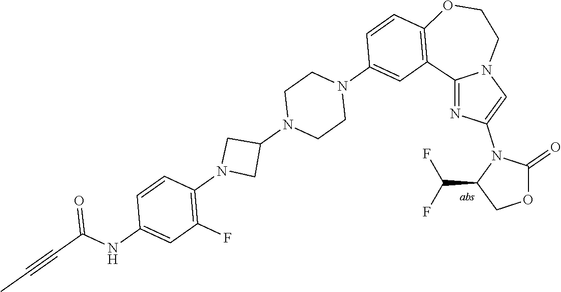 [SMILES: CC#CC(=O)Nc1ccc(N2CC(N3CCN(c4ccc5c(c4)-c4nc(N6C(=O)OC[C@H]6C(F)F)cn4CCO5)CC3)C2)c(F)c1] | ¹H NMR (400 MHz, DMSO-d₆) δ 10.49 (s, 1H), 7.76 (d, J = 2.8 Hz, 1H), 7.43-7.37 (m, 1H), 7.34 (s, 1H), 7.18 (dd, J = 8.7, 2.3 Hz, 1H), 6.96 (dd, J = 8.9, 2.9 Hz, 1H), 6.91 (d, J = 8.9 Hz, 1H), 6.82-6.48 (m, 2H), 5.08-4.96 (m, 1H), 4.62 (t, J = 9.1 Hz, 1H), 4.56 (dd, J = 9.4, 3.8 Hz, 1H), 4.41-4.36 (m, 4H), 4.01-3.96 (m, 2H), 3.68 (t, J = 6.5 Hz, 2H), 3.10 (m, 1H), 2.51 (m, 4H), 2.02 (s, 3H). |
| 150 | 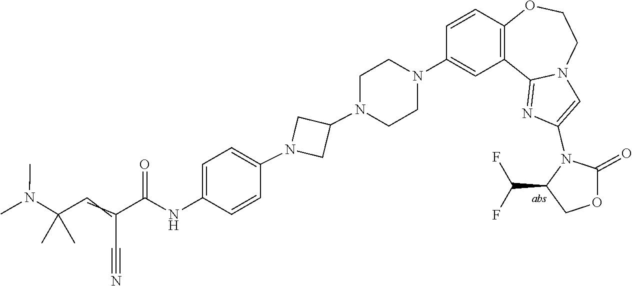 [SMILES: CN(C)C(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1] | ¹H NMR (400 MHz, DMSO-d₆) δ 9.89 (s, 1H), 7.77 (d, J = 2.9 Hz, 1H), 7.44-7.31 (m, 4H), 6.96 (dd, J = 9.0, 2.9 Hz, 1H), 6.91 (d, J = 8.9 Hz, 1H), 6.83-6.52 (m, 1H), 6.46-6.40 (m, 2H), 5.02 (m, 1H), 4.66-4.52 (m, 2H), 4.42-4.35 (m, 4H), 3.93 (t, J = 7.1 Hz, 2H), 3.65-3.57 (m, 2H), 3.36 (s, 1H), 3.11 (s, 4H), 2.54-2.51 (m, 4H), 2.20 (s, 6H), 1.27 (s, 6H). |
| 151 | 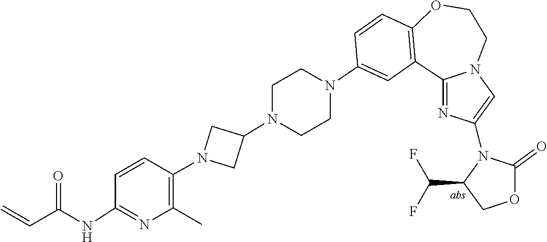 [SMILES: C=CC(=O)Nc1ccc(N2CC(N3CCN(c4ccc5c(c4)-c4nc(N6C(=O)OC[C@H]6C(F)F)cn4CCO5)CC3)C2)c(C)n1] | ¹H NMR (400 MHz, DMSO-d₆) δ 10.37 (s, 1H), 7.91 (d, J = 8.5 Hz, 1H), 7.77 (d, J = 2.9 Hz, 1H), 7.34 (s, 1H), 6.98-6.91 (m, 3H), 6.83- 6.53 (m, 2H), 6.23 (dd, J = 17.0, 2.2 Hz, 1H), 5.69 (dd, J = 10.1, 2.1 Hz, 1H), 5.07-4.96 (m, 1H), 4.62 (t, J = 9.1 Hz, 1H), 4.56 (dd, J = 9.4, 3.8 Hz, 1H), 4.40 (d, J = 6.3 Hz, 2H), 4.37 (d, J = 5.6 Hz, 2H), 4.00 (t, J = 6.8 Hz, 2H), 3.68 (s, 2H), 3.36 (s, 1H), 3.11 (s, 4H), 2.53 (m, 4H), 2.31 (s, 3H). |
| 152 | 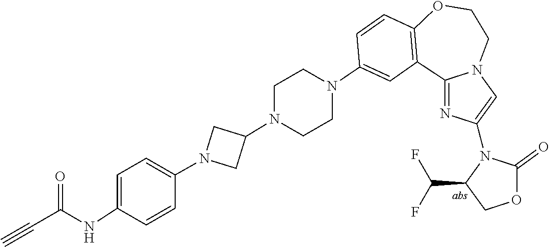 [SMILES: C#CC(=O)Nc1ccc(N2CC(N3CCN(c4ccc5c(c4)-c4nc(N6C(=O)OC[C@H]6C(F)F)cn4CCO5)CC3)C2)cc1] | ¹H NMR (400 MHz, DMSO-d₆) δ 10.51 (s, 1H), 7.76 (d, J = 2.9 Hz, 1H), 7.45-7.35 (m, 2H), 7.34 (s, 1H), 6.97-6.90 (m, 2H), 6.82- 6.52 (m, 1H), 6.45-6.35 (m, 2H), 5.08-4.96 (m, 1H), 4.65-4.54 (m, 2H), 4.40 (d, J = 5.4 Hz, 2H), 4.37 (d, J = 5.6 Hz, 2H), 4.30 (s, 1H), 3.91 (t, J = 7.0 Hz, 2H), 3.64-3.56 (m, 2H), 3.37 (m, 1H), 3.11 (s, 4H), 2.50-2.45 (m, 4H). |
| 153 | 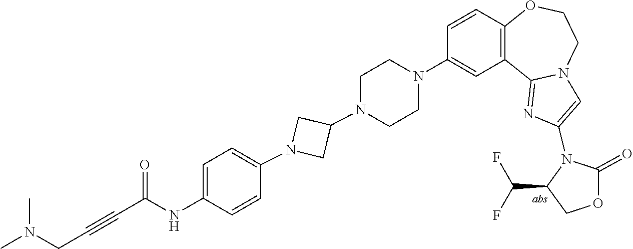 [SMILES: CN(C)CC#CC(=O)Nc1ccc(N2CC(N3CCN(c4ccc5c(c4)-c4nc(N6C(=O)OC[C@H]6C(F)F)cn4CCO5)CC3)C2)cc1] | ¹H NMR (400 MHz, DMSO-d₆) δ 10.33 (s, 1H), 7.77 (d, J = 2.9 Hz, 1H), 7.44-7.38 (m, 2H), 7.34 (s, 1H), 6.96 (dd, J = 9.0, 2.9 Hz, 1H), 6.91 (d, J = 8.9 Hz, 1H), 6.82-6.51 (m, 1H), 6.43-6.37 (m, 2H), 5.08-4.96 (m, 1H), 4.62 (t, J = 9.1 Hz, 1H), 4.56 (dd, J = 9.4, 3.8 Hz, 1H), 4.41-4.35 (m, 4H), 3.92 (t, J = 7.0 Hz, 2H), 3.63-3.57 (m, 2H), 3.46 (d, J = 3.3 Hz, 1H), 3.42 (s, 2H), 3.11 (s, 4H), 2.55-2.51 (m, 4H), 2.24 (s, 6H). |
| 154 | 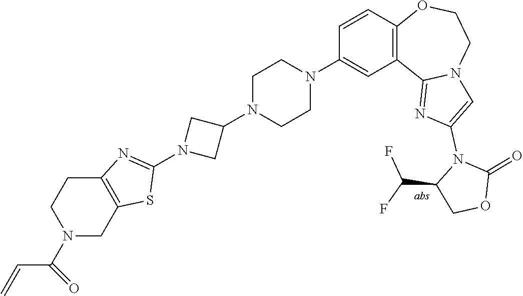 [SMILES: C=CC(=O)N1CCc2nc(N3CC(N4CCN(c5ccc6c(c5)-c5nc(N7C(=O)OC[C@H]7C(F)F)cn5CCO6)CC4)C3)sc2C1] | ¹H NMR (400 MHz, DMSO-d₆) δ 7.77 (d, J = 2.9 Hz, 1H), 7.34 (s, 1H), 6.97-6.52 (m, 4H), 6.13 (dd, J = 16.7, 2.3 Hz, 1H), 5.71 (d, J = 10.5 Hz, 1H), 5.07-4.95 (m, 1H), 4.66-4.54 (m, 4H), 4.41-4.35 (m, 4H), 4.03 (t, J = 7.6 Hz, 2H), 3.87-3.77 (m, 4H), 3.42 (s, 2H), 3.23 (s, 1H), 3.13-3.08 (m, 4H), 2.54-2.51 (m, 4H). |
| 155 | 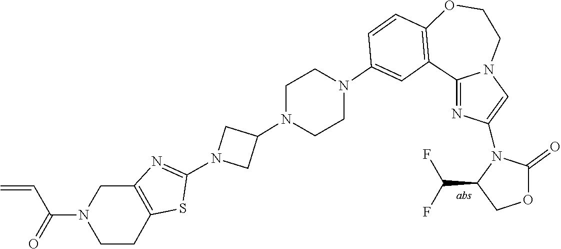 [SMILES: C=CC(=O)N1CCc2sc(N3CC(N4CCN(c5ccc6c(c5)-c5nc(N7C(=O)OC[C@H]7C(F)F)cn5CCO6)CC4)C3)nc2C1] | ¹H NMR (400 MHz, DMSO-d₆) δ 7.77 (d, J = 2.9 Hz, 1H), 7.34 (s, 1H), 6.99-6.52 (m, 4H), 6.13 (dd, J = 16.7, 2.4 Hz, 1H), 5.75-5.66 (m, 1H), 5.08-4.94 (m, 1H), 4.64-4.43 (m, 4H), 4.41-4.35 (m, 4H), 4.04 (dd, J = 8.2, 7.0 Hz, 2H), 3.89-3.76 (m, 4H), 3.39 (m, 1H), 3.14-3.06 (m, 4H), 2.67 (m, 2H), 2.52 (d, J = 4.5 Hz, 4H). |
| 156 | 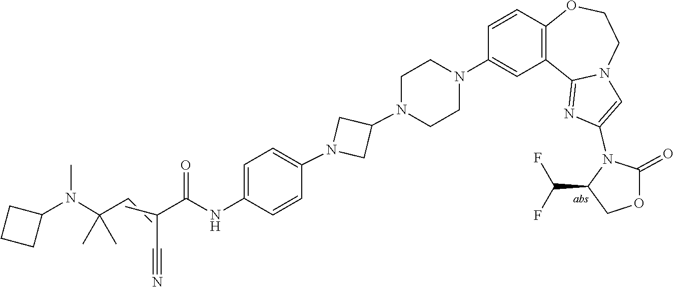 | ¹H NMR (400 MHz, DMSO-d₆) δ 9.82 (s, 1H), 7.73 (d, J = 2.9 Hz, 1H), 7.36 (d, J = 8.8 Hz, 2H), 7.31 (d, J = 9.9 Hz, 2H), 6.92 (dd, J = 9.0, 2.9 Hz, 1H), 6.87 (d, J = 8.9 Hz, 1H), 6.63 (d, J = 2.1 Hz, 1H), 6.41-6.37 (m, 2H), 5.02-4.92 (m, 1H), 4.58 (t, J = 9.1 Hz, 1H), 4.52 (dd, J = 9.4, 3.8 Hz, 1H), 4.37-4.31 (m, 4H), 4.18 (t, J = 6.6 Hz, 1H), 3.89 (t, J = 7.1 Hz, 2H), 3.57 (dd, J = 7.5, 5.4 Hz, 2H), 3.28 (m, 1H), 3.07 (s, 4H), 2.48 (m, 4H), 2.13 (s, 3H), 2.03-1.94 (m, 2H), 1.89-1.79 (m, 2H), 1.51-1.43 (m, 2H), 1.21 (s, 6H). |
| 159 | 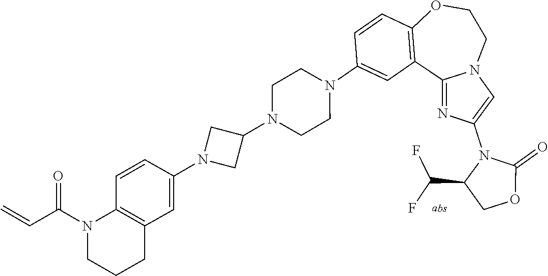 [SMILES: C=CC(=O)N1CCCc2cc(N3CC(N4CCN(c5ccc6c(c5)-c5nc(N7C(=O)OC[C@H]7C(F)F)cn5CCO6)CC4)C3)ccc21] | ¹H NMR (400 MHz, DMSO-d₆) δ 7.72 (t, J = 3.1 Hz, 1H), 7.35- 7.22 (m, 1H), 7.04-6.82 (m, 3H), 6.55 (d, J = 56.4 Hz, 2H), 6.33- 6.20 (m, 2H), 6.13-6.11 (m, 1H), 5.59 (d, J = 10.1 Hz, 1H), 4.96 (m, 1H), 4.67-4.44 (m, 2H), 4.40-4.27 (m, 4H), 3.89 (m, 2H), 3.69- 3.51 (m, 4H), 3.25 (m, 3H), 3.07 (m, 4H), 2.56 (m, 4H), 1.79-1.77 (m, 2H). |
| 160 | 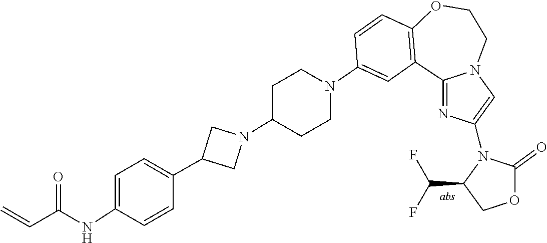 [SMILES: C=CC(=O)Nc1ccc(C2CN(C3CCN(c4ccc5c(c4)-c4nc(N6C(=O)OC[C@H]6C(F)F)cn4CCO5)CC3)C2)cc1] | ¹H NMR (400 MHz, DMSO-d₆) δ 10.15 (s, 1H), 7.75 (d, J = 2.9 Hz, 1H), 7.61 (d, J = 8.1 Hz, 2H), 7.31 (d, J = 7.5 Hz, 3H), 6.93 (dd, J = 9.0, 3.0 Hz, 1H), 6.87 (d, J = 8.8 Hz, 1H), 6.64 (d, J = 2.6 Hz, 1H), 6.40 (dd, J = 17.0, 10.1 Hz, 1H), 6.20 (dd, J = 17.0, 2.1 Hz, 1H), 5.70 (dd, J = 10.1, 2.1 Hz, 1H), 5.04-4.92 (m, 1H), 4.69-4.45 (m, 2H), 4.40-4.29 (m, 4H), 3.61 (m, 6H), 2.67 (m, 2H), 1.84 (s, 2H), 1.41 (s, 2H), 1.20 (m, 2H). |
| 161 | 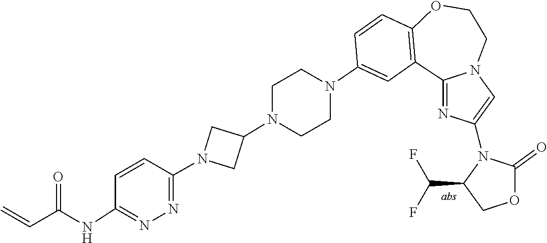 [SMILES: C=CC(=O)Nc1ccc(N2CC(N3CCN(c4ccc5c(c4)-c4nc(N6C(=O)OC[C@H]6C(F)F)cn4CCO5)CC3)C2)nn1] | ¹H NMR (400 MHz, DMSO-d₆) δ 10.86 (s, 1H), 8.10 (d, J = 9.6 Hz, 1H), 7.73 (d, J = 2.9 Hz, 1H), 7.30 (s, 1H), 6.96-6.83 (m, 3H), 6.63 (s, 1H), 6.58-6.48 (m, 1H), 6.24 (dd, J = 17.0, 2.0 Hz, 1H), 5.73 (dd, J = 10.2, 2.0 Hz, 1H), 5.02-4.91 (m, 1H), 4.62-4.48 (m, 2H), 4.39-4.30 (m, 4H), 4.11-4.03 (m, 2H), 3.86 (dd, J = 8.4, 5.2 Hz, 2H), 3.28 (m, 1H), 3.08 (s, 4H), 2.51 (m, 4H). |
| 162 | 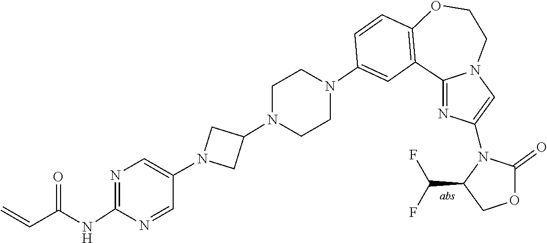 [SMILES: C=CC(=O)Nc1ncc(N2CC(N3CCN(c4ccc5c(c4)-c4nc(N6C(=O)OC[C@H]6C(F)F)cn4CCO5)CC3)C2)cn1] | ¹H NMR (400 MHz, DMSO-d₆) δ 10.41 (s, 1H), 7.95 (s, 2H), 7.73 (d, J = 2.9 Hz, 1H), 7.30 (s, 1H), 6.97-6.85 (m, 2H), 6.59 (d, J = 37.7 Hz, 1H), 6.53-6.46 (m, 1H), 6.20 (dd, J = 17.0, 2.1 Hz, 1H), 5.68 (dd, J = 10.2, 2.0 Hz, 1H), 5.04-4.91 (m, 1H), 4.63-4.49 (m, 2H), 4.39-4.30 (m, 4H), 4.00 (t, J = 7.3 Hz, 2H), 3.77-3.70 (m, 2H), 3.29 (m, 1H), 3.08 (s, 4H), 2.50 (m, 4H). |
| 163 | 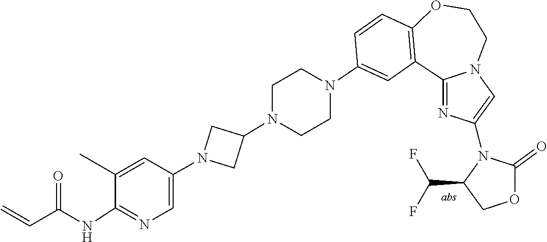 [SMILES: C=CC(=O)Nc1ncc(N2CC(N3CCN(c4ccc5c(c4)-c4nc(N6C(=O)OC[C@H]6C(F)F)cn4CCO5)CC3)C2)cc1C] | ¹H NMR (400 MHz, DMSO-d₆) δ 9.83 (s, 1H), 7.73 (d, J = 2.9 Hz, 1H), 7.47 (d, J = 2.7 Hz, 1H), 7.30 (s, 1H), 6.96-6.84 (m, 2H), 6.79- 6.47 (m, 2H), 6.38 (dd, J = 17.0, 10.2 Hz, 1H), 6.16 (dd, J = 17.1, 2.1 Hz, 1H), 5.66 (dd, J = 10.2, 2.1 Hz, 1H), 5.04-4.92 (m, 1H), 4.58 (t, J = 9.1 Hz, 1H), 4.52 (dd, J = 9.4, 3.8 Hz, 1H), 4.40-4.30 (m, 4H), 3.96 (t, J = 7.2 Hz, 2H), 3.67 (dd, J = 7.7, 5.4 Hz, 2H), 3.41 (d, J = 15.1 Hz, 1H), 3.08 (s, 4H), 2.53-2.48 (m, 4H), 2.05 (s, 3H). |
| 164 | 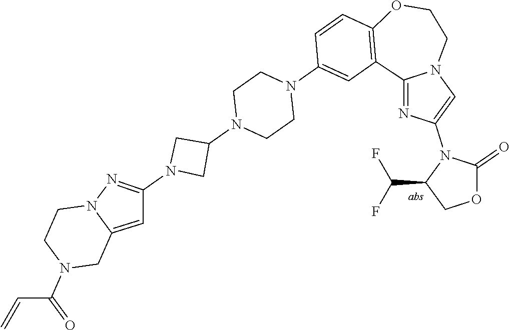 [SMILES: C=CC(=O)N1CCn2nc(N3CC(N4CCN(c5ccc6c(c5)-c5nc(N7C(=O)OC[C@H]7C(F)F)cn5CCO6)CC4)C3)cc2C1] | ¹H NMR (400 MHz, DMSO-d₆) δ 7.76 (d, J = 2.8 Hz, 1H), 7.33 (s, 1H), 6.98-6.82 (m, 3H), 6.82-6.51 (m, 1H), 6.17 (dd, J = 16.7, 2.3 Hz, 1H), 5.75 (dd, J = 10.5, 2.2 Hz, 1H), 5.38 (d, J = 11.3 Hz, 1H), 5.08-4.95 (m, 1H), 4.79-4.54 (m, 4H), 4.41-4.35 (m, 4H), 3.96 (d, J = 30.1 Hz, 4H), 3.83 (t, J = 7.1 Hz, 2H), 3.59 (t, J = 6.4 Hz, 2H), 3.25 (d, J = 5.8 Hz, 1H), 3.09 (s, 4H), 2.46 (m, 4H). |
| 165 | 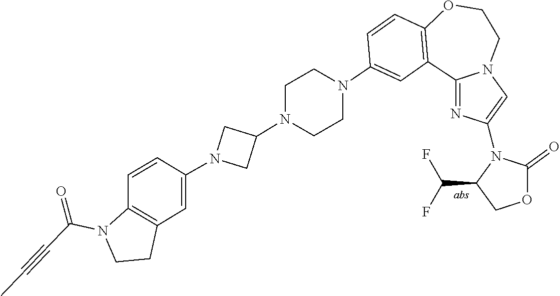 [SMILES: CC#CC(=O)N1CCc2cc(N3CC(N4CCN(c5ccc6c(c5)-c5nc(N7C(=O)OC[C@H]7C(F)F)cn5CCO6)CC4)C3)ccc21] | ¹H NMR (400 MHz, DMSO-d₆) δ 7.79 (d, J = 8.6 Hz, 1H), 7.76 (d, J = 2.9 Hz, 1H), 7.34 (s, 1H), 6.96 (dd, J = 9.0, 2.9 Hz, 1H), 6.91 (d, J = 8.9 Hz, 1H), 6.67 (m, 1H), 6.39 (s, 1H), 6.24 (dd, J = 8.6, 2.4 Hz, 1H), 5.03 (s, 1H), 4.64-4.54 (m, 2H), 4.41-4.35 (m, 4H), 4.16 (t, J = 8.3 Hz, 2H), 3.91 (t, J = 7.1 Hz, 2H), 3.61-3.57 (m, 2H), 3.37 (s, 2H), 3.30 (s, 2H), 3.09 (m, 7H), 2.06 (s, 3H). |
| 166 | 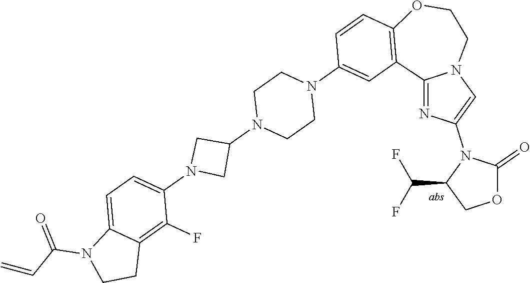 [SMILES: C=CC(=O)N1CCc2c1ccc(N1CC(N3CCN(c4ccc5c(c4)-c4nc(N6C(=O)OC[C@H]6C(F)F)cn4CCO5)CC3)C1)c2F] | ¹H NMR (400 MHz, DMSO-d₆) δ 7.93-7.72 (m, 2H), 7.34 (s, 1H), 6.98-6.89 (m, 2H), 6.69 (dd, J = 15.8, 9.4 Hz, 2H), 6.39 (t, J = 8.9 Hz, 1H), 6.26 (dd, J = 16.7, 2.2 Hz, 1H), 5.77 (dd, J = 10.3, 2.3 Hz, 1H), 5.14-4.91 (m, 1H), 4.62 (t, J = 9.1 Hz, 1H), 4.56 (dd, J = 9.3, 3.8 Hz, 1H), 4.43-4.32 (m, 4H), 4.23 (t, J = 8.3 Hz, 2H), 4.00 (t, J = 6.6 Hz, 2H), 3.75-3.58 (m, 4H), 3.51-3.47 (m, 1H), 3.14-3.06 (m, 4H), 2.49-2.38 (m, 4H). |
| 167 | 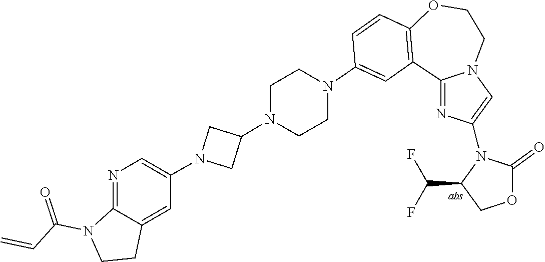 [SMILES: C=CC(=O)N1CCc2cc(N3CC(N4CCN(c5ccc6c(c5)-c5nc(N7C(=O)OC[C@H]7C(F)F)cn5CCO6)CC4)C3)cnc21] | ¹H NMR (400 MHz, DMSO-d₆) δ 7.97 (dd, J = 17.0, 10.3 Hz, 1H), 7.77 (d, J = 2.9 Hz, 1H), 7.37-7.31 (m, 2H), 6.98-6.89 (m, 3H), 6.84-6.51 (m, 1H), 6.29 (dd, J = 17.1, 2.5 Hz, 1H), 5.76 (dd, J = 10.4, 2.5 Hz, 1H), 5.08-4.94 (m, 1H), 4.65-4.53 (m, 2H), 4.42- 4.35 (m, 4H), 4.09-3.90 (m, 4H), 3.65 (dd, J = 7.5, 5.4 Hz, 2H), 3.31 (s, 1H), 3.11 (s, 4H), 3.00 (t, J = 8.2 Hz, 2H), 2.53 (m, 4H). |
| 168 | 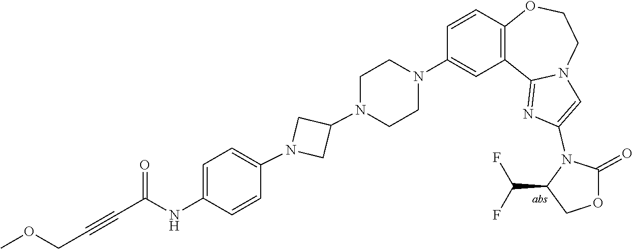 [SMILES: COCC#CC(=O)Nc1ccc(N2CC(N3CCN(c4ccc5c(c4)-c4nc(N6C(=O)OC[C@H]6C(F)F)cn4CCO5)CC3)C2)cc1] | ¹H NMR (400 MHz, DMSO-d₆) δ 10.46 (s, 1H), 7.77 (d, J = 2.9 Hz, 1H), 7.43-7.38 (m, 2H), 7.34 (s, 1H), 6.98-6.90 (m, 2H), 6.82- 6.52 (m, 1H), 6.40 (d, J = 8.9 Hz, 2H), 5.08-4.95 (m, 1H), 4.65- 4.53 (m, 2H), 4.39 (t, J = 6.7 Hz, 4H), 4.31 (s, 2H), 3.92 (t, J = 7.0 Hz, 2H), 3.60 (t, J = 6.4 Hz, 2H), 3.34 (s, 4H), 3.11 (s, 4H), 2.55- 2.50 (m, 4H). |
| 169 | 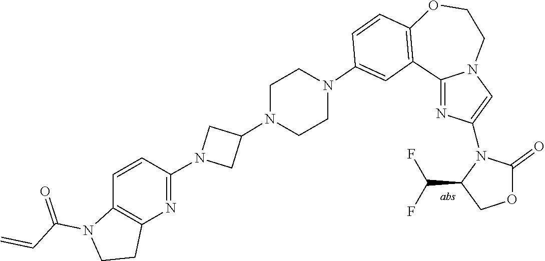 [SMILES: C=CC(=O)N1CCc2nc(N3CC(N4CCN(c5ccc6c(c5)-c5nc(N7C(=O)OC[C@H]7C(F)F)cn5CCO6)CC4)C3)ccc21] | ¹H NMR (400 MHz, DMSO-d₆) δ 8.25 (d, J = 8.7 Hz, 1H), 7.79 (d, J = 2.9 Hz, 1H), 7.36 (s, 1H), 7.32-6.55 (m, 4H), 6.33-6.20 (m, 2H), 5.79 (dd, J = 10.2, 2.3 Hz, 1H), 5.07-4.94 (m, 1H), 4.67-4.54 (m, 2H), 4.44-4.36 (m, 4H), 4.26-4.03 (m, 5H), 3.61 (m, 3H), 3.13 (m, 5H), 2.55-2.50 (m, 4H). |
| 170 | 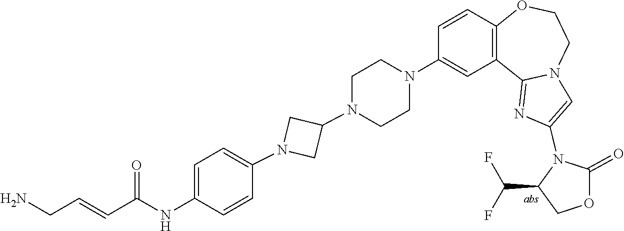 [SMILES: NC/C=C/C(=O)Nc1ccc(N2CC(N3CCN(c4ccc5c(c4)-c4nc(N6C(=O)OC[C@H]6C(F)F)cn4CCO5)CC3)C2)cc1] | ¹H NMR (400 MHz, DMSO-d₆) δ 9.87 (d, J = 7.0 Hz, 1H), 7.77 (d, J = 6.8 Hz, 1H), 7.47 (d, J = 8.2 Hz, 2H), 7.34 (d, J = 7.7 Hz, 1H), 7.02-6.87 (m, 2H), 6.83-6.52 (m, 2H), 6.40 (d, J = 8.2 Hz, 2H), 6.27-6.01 (m, 2H), 5.71 (d, J = 9.6 Hz, 1H), 5.11-4.93 (m, 1H), 4.66-4.54 (m, 2H), 4.38 (m, 4H), 3.91 (m, 2H), 3.58 (m, 3H), 3.18- 3.06 (m, 4H), 2.55-2.50 (m, 4H). |
| 171 | 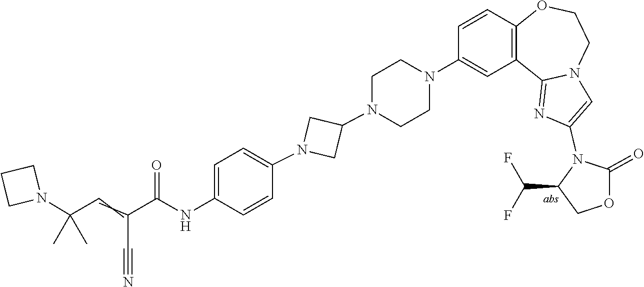 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)N1CCC1] | ¹H NMR (400 MHz, DMSO-d₆) δ 9.93 (s, 1H), 7.77 (d, J = 2.8 Hz, 1H), 7.43-7.32 (m, 4H), 6.96 (dd, J = 9.0, 2.8 Hz, 1H), 6.91 (d, J = 8.9 Hz, 1H), 6.83-6.52 (m, 1H), 6.45-6.40 (m, 2H), 5.09-4.94 (m, 1H), 4.62 (t, J = 9.1 Hz, 1H), 4.56 (dd, J = 9.4, 3.8 Hz, 1H), 4.41- 4.38 (m, 2H), 4.38-4.35 (m, 2H), 3.93 (t, J = 7.0 Hz, 2H), 3.61 (dd, J = 7.5, 5.4 Hz, 2H), 3.30 (m, 1H), 3.21 (t, J = 6.9 Hz, 4H), 3.14- 3.07 (m, 4H), 2.52 (m, 4H), 1.98-1.82 (m, 2H), 1.22 (s, 6H). |
| 172 | 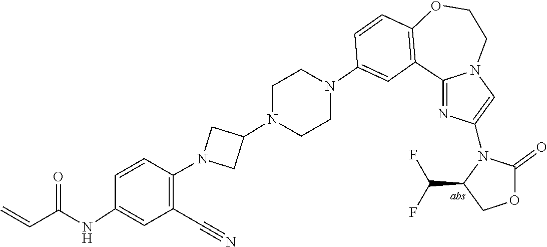 [SMILES: C=CC(=O)Nc1ccc(N2CC(N3CCN(c4ccc5c(c4)-c4nc(N6C(=O)OC[C@H]6C(F)F)cn4CCO5)CC3)C2)c(C#N)c1] | ¹H NMR (400 MHz, DMSO-d₆) δ 10.13 (s, 1H), 7.88 (d, J = 2.4 Hz, 1H), 7.77 (d, J = 2.8 Hz, 1H), 7.60 (dd, J = 9.1, 2.5 Hz, 1H), 7.34 (s, 1H), 6.96 (dd, J = 9.0, 2.9 Hz, 1H), 6.92 (d, J = 9.0 Hz, 1H), 6.83- 6.50 (m, 2H), 6.36 (dd, J = 17.0, 10.0 Hz, 1H), 6.23 (dd, J = 17.0, 2.2 Hz, 1H), 5.74 (dd, J = 10.0, 2.2 Hz, 1H), 5.08-4.96 (m, 1H), 4.62 (t, J = 9.1 Hz, 1H), 4.56 (dd, J = 9.4, 3.8 Hz, 1H), 4.41-4.35 (m, 4H), 4.20 (t, J = 7.5 Hz, 2H), 3.91 (dd, J = 8.1, 5.2 Hz, 2H), 3.51 (s, 1H), 3.12 (m, 4H), 2.53 (m, 4H). |
| 173 | 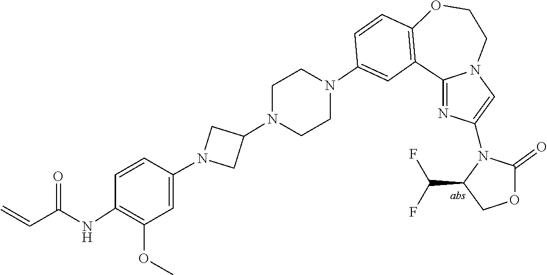 [SMILES: C=CC(=O)Nc1ccc(N2CC(N3CCN(c4ccc5c(c4)-c4nc(N6C(=O)OC[C@H]6C(F)F)cn4CCO5)CC3)C2)cc1OC] | ¹H NMR (400 MHz, DMSO-d₆) δ 9.10 (s, 1H), 7.77 (d, J = 2.8 Hz, 1H), 7.65 (d, J = 8.5 Hz, 1H), 7.34 (s, 1H), 6.96 (dd, J = 9.0, 2.9 Hz, 1H), 6.92 (d, J = 8.8 Hz, 1H), 6.84-6.51 (m, 2H), 6.15 (dd, J = 17.0, 2.2 Hz, 1H), 6.11 (d, J = 2.4 Hz, 1H), 5.99 (dd, J = 8.6, 2.4 Hz, 1H), 5.63 (dd, J = 10.1, 2.3 Hz, 1H), 5.08-4.96 (m, 1H), 4.65-4.54 (m, 2H), 4.42-4.35 (m, 4H), 3.95 (t, J = 7.1 Hz, 2H), 3.80 (s, 3H), 3.64 (dd, J = 7.5, 5.4 Hz, 2H), 3.33-3.31 (m, 1H), 3.15-3.07 (m, 4H), 2.53 (s, 4H). |
| 174 | 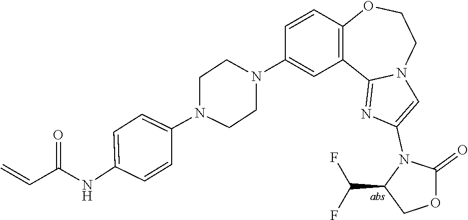 [SMILES: C=CC(=O)Nc1ccc(N2CCN(c3ccc4c(c3)-c3nc(N5C(=O)OC[C@H]5C(F)F)cn3CCO4)CC2)cc1] | ¹H NMR (400 MHz, DMSO-d₆) δ 9.90 (s, 1H), 7.80 (d, J = 3.0 Hz, 1H), 7.56-7.47 (m, 2H), 7.32 (s, 1H), 7.02-6.88 (m, 4H), 6.80- 6.52 (m, 1H), 6.37 (dd, J = 17.0, 10.1 Hz, 1H), 6.17 (dd, J = 17.0, 2.1 Hz, 1H), 5.74-5.62 (m, 1H), 5.07-4.94 (m, 1H), 4.64-4.50 (m, 2H), 4.43-4.31 (m, 4H), 3.21 (m, 8H). |
| 175 | 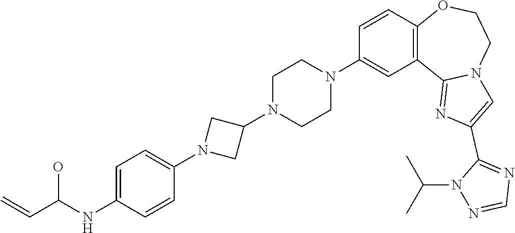 [SMILES: C=CC(O)Nc1ccc(N2CC(N3CCN(c4ccc5c(c4)-c4nc(-c6ncnn6C(C)C)cn4CCO5)CC3)C2)cc1] | ¹H NMR (400 MHz, DMSO-d₆) δ 9.85 (s, 1H), 7.92 (d, J = 3.1 Hz, 1H), 7.91-7.89 (m, 2H), 7.49-7.45 (m, 2H), 7.02 (dd, J = 9.0, 3.0 Hz, 1H), 6.95 (d, J = 8.9 Hz, 1H), 6.45-6.35 (m, 3H), 6.18 (dd, J = 17.0, 2.2 Hz, 1H), 5.80-5.72 (m, 1H), 5.67 (dd, J = 10.1, 2.1 Hz, 1H), 4.51-4.47 (m, 2H), 4.44-4.40 (m, 2H), 3.92 (t, J = 7.1 Hz, 2H), 3.62-3.59 (m, 2H), 3.52-3.40 (m, 1H), 3.13 (m, 4H), 2.54 (m, 4H), 1.50 (d, J = 6.6 Hz, 6H). |
| 176 | 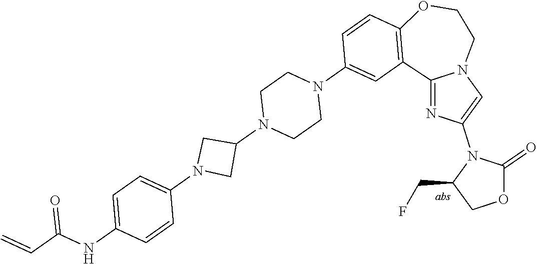 [SMILES: C=CC(=O)Nc1ccc(N2CC(N3CCN(c4ccc5c(c4)-c4nc(N6C(=O)OC[C@H]6CF)cn4CCO5)CC3)C2)cc1] | ¹H NMR (400 MHz, DMSO-d₆) δ 9.85 (s, 1H), 7.76 (d, J = 2.9 Hz, 1H), 7.48 (d, J = 8.9 Hz, 2H), 7.33 (s, 1H), 6.98-6.88 (m, 2H), 6.45- 6.34 (m, 3H), 6.18 (dd, J = 17.0, 2.2 Hz, 1H), 5.67 (dd, J = 10.1, 2.2 Hz, 1H), 5.09-4.90 (m, 1H), 4.84-4.59 (m, 3H), 4.44-4.35 (m, 5H), 3.92 (t, J = 7.0 Hz, 2H), 3.63-3.57 (m, 2H), 3.34-3.18 (m, 1H), 3.10 (s, 4H), 2.55-2.50 (m, 4H). |
| 177 | 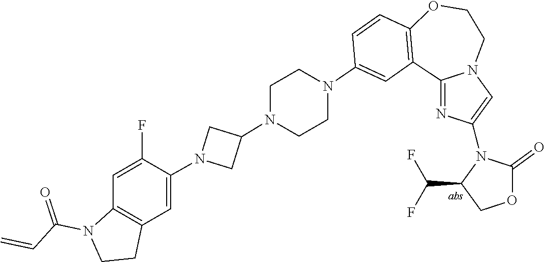 [SMILES: C=CC(=O)N1CCc2cc(N3CC(N4CCN(c5ccc6c(c5)-c5nc(N7C(=O)OC[C@H]7C(F)F)cn5CCO6)CC4)C3)c(F)cc21] | ¹H NMR (400 MHz, DMSO-d₆) δ 7.88-7.67 (m, 2H), 7.30 (s, 1H), 6.98-6.84 (m, 2H), 6.77-6.45 (m, 3H), 6.23 (d, J = 15.8 Hz, 1H), 5.74 (d, J = 10.1 Hz, 1H), 5.00 (m, 1H), 4.67-4.47 (m, 2H), 4.35 (m, 4H), 4.16 (t, J = 8.3 Hz, 2H), 3.96 (t, J = 7.0 Hz, 2H), 3.64 (t, J = 6.8 Hz, 2H), 3.30 (m, 1H), 3.06 (m, 6H). |
| 180 | 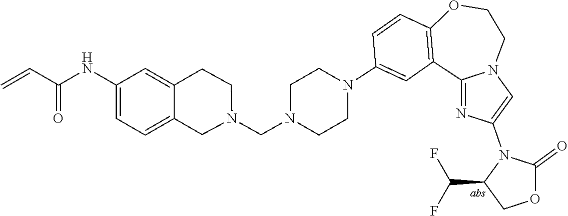 [SMILES: C=CC(=O)Nc1ccc2c(c1)CCN(CN1CCN(c3ccc4c(c3)-c3nc(N5C(=O)OC[C@H]5C(F)F)cn3CCO4)CC1)C2] | ¹H NMR (400 MHz, DMSO-d₆) δ 10.02 (s, 1H), 7.78 (d, J = 2.9 Hz, 1H), 7.46 (d, J = 2.1 Hz, 1H), 7.37 (dd, J = 8.3, 2.2 Hz, 1H), 7.33 (s, 1H), 7.03-6.83 (m, 3H), 6.81-6.50 (m, 1H), 6.48-6.31 (m, 1H), 6.29-6.19 (m, 1H), 5.72 (dd, J = 10.1, 2.1 Hz, 1H), 5.11-4.94 (m, 1H), 4.64-4.53 (m, 2H), 4.41-4.34 (m, 4H), 3.62-3.51 (m, 4H), 2.79 (t, J = 5.8 Hz, 2H), 2.69-2.60 (m, 4H), 2.36 (d, J = 7.0 Hz, 2H), 1.87-1.72 (m, 3H), 1.29 (t, J = 11.8 Hz, 2H). |
| 181 | 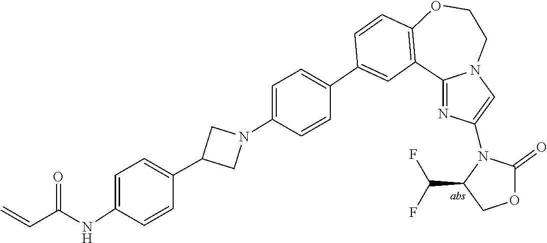 [SMILES: C=CC(=O)Nc1ccc(C2CN(c3ccc(-c4ccc5c(c4)-c4nc(N6C(=O)OC[C@H]6C(F)F)cn4CCO5)cc3)C2)cc1] | ¹H NMR (400 MHz, DMSO-d₆) δ 10.09 (s, 1H), 8.42 (d, J = 2.4 Hz, 1H), 7.62 (d, J = 8.4 Hz, 2H), 7.50-7.41 (m, 3H), 7.37-7.28 (m, 3H), 7.03 (d, J = 8.5 Hz, 1H), 6.81-6.51 (m, 3H), 6.39 (dd, J = 17.0, 10.1 Hz, 1H), 6.21 (dd, J = 17.0, 2.1 Hz, 1H), 5.70 (dd, J = 10.1, 2.1 Hz, 1H), 5.08-4.96 (m, 1H), 4.65-4.49 (m, 2H), 4.43 (s, 4H), 4.24 (t, J = 7.6 Hz, 2H), 3.92-3.89 (m, 1H), 3.78 (t, J = 6.6 Hz, 2H). |
| 183 | 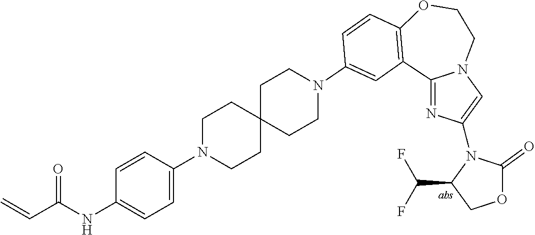 [SMILES: C=CC(=O)Nc1ccc(N2CCC3(CC2)CCN(c2ccc4c(c2)-c2nc(N5C(=O)OC[C@H]5C(F)F)cn2CCO4)CC3)cc1] | ¹H NMR (400 MHz, DMSO-d₆) δ 9.85 (s, 1H), 7.75 (d, J = 3.0 Hz, 1H), 7.51-7.43 (m, 2H), 7.30 (s, 1H), 6.96-6.83 (m, 4H), 6.75- 6.47 (m, 1H), 6.36 (dd, J = 17.0, 10.1 Hz, 1H), 6.26 (m, 1H), 5.65 (dd, J = 10.1, 2.1 Hz, 1H), 5.03-4.95 (m, 1H), 4.62-4.48 (m, 2H), 4.39-4.29 (m, 4H), 3.07 (m, 8H), 1.58-1.55 (m, 8H). |
| 186 | 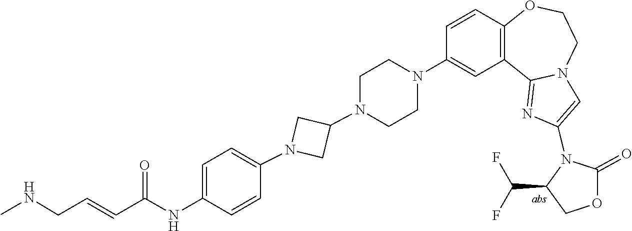 [SMILES: CNC/C=C/C(=O)Nc1ccc(N2CC(N3CCN(c4ccc5c(c4)-c4nc(N6C(=O)OC[C@H]6C(F)F)cn4CCO5)CC3)C2)cc1] | ¹H NMR (400 MHz, DMSO-d₆) δ 9.74 (s, 1H), 7.73 (d, J = 2.9 Hz, 1H), 7.42 (d, J = 8.6 Hz, 2H), 7.30 (s, 1H), 6.96-6.84 (m, 2H), 6.67- 6.65 (m, 2H), 6.37 (d, J = 8.8 Hz, 2H), 6.17 (d, J = 15.3 Hz, 1H), 5.05-4.90 (m, 1H), 4.62-4.48 (m, 2H), 4.41-4.28 (m, 4H), 3.88 (t, J = 7.0 Hz, 2H), 3.55 (m, 4H), 3.31 (m, 1H), 3.07 (m, 4H), 2.47 (m, 4H), 2.28 (s, 3H). |
| 194 | 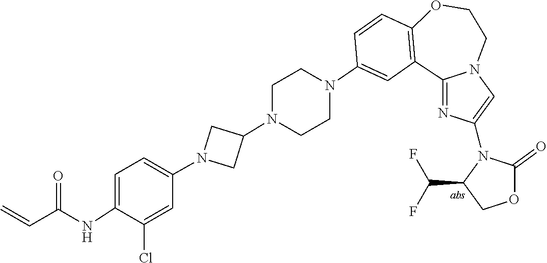 [SMILES: C=CC(=O)Nc1ccc(N2CC(N3CCN(c4ccc5c(c4)-c4nc(N6C(=O)OC[C@H]6C(F)F)cn4CCO5)CC3)C2)cc1Cl] | ¹H NMR (400 MHz, DMSO-d₆) δ 9.45 (s, 1H), 7.73 (d, J = 2.9 Hz, 1H), 7.34 (d, J = 8.7 Hz, 1H), 7.30 (s, 1H), 6.96 - 6.84 (m, 2H), 6.78- 6.50 (m, 1H), 6.50-6.42 (m, 2H), 6.37 (dd, J = 8.7, 2.6 Hz, 1H), 6.17 (dd, J = 17.0, 2.1 Hz, 1H), 5.68 (dd, J = 10.2, 2.1 Hz, 1H), 5.04- 4.92 (m, 1H), 4.58 (t, J = 9.1 Hz, 1H), 4.52 (dd, J = 9.4, 3.8 Hz, 1H), 4.40-4.29 (m, 4H), 3.92 (t, J = 7.2 Hz, 2H), 3.63 (dd, J = 7.7, 5.3 Hz, 2H), 3.30 (m, 1H), 3.12-3.03 (m, 4H), 2.49 (s, 4H). |
| 195 | 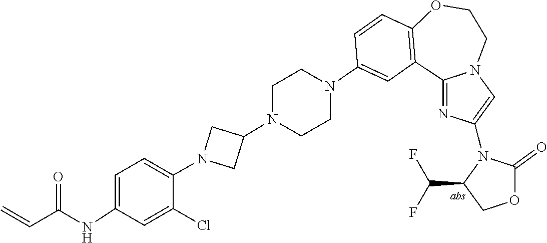 [SMILES: C=CC(=O)Nc1ccc(N2CC(N3CCN(c4ccc5c(c4)-c4nc(N6C(=O)OC[C@H]6C(F)F)cn4CCO5)CC3)C2)c(Cl)c1] | ¹H NMR (400 MHz, DMSO-d₆) δ 10.01 (s, 1H), 7.71 (dd, J = 15.8, 2.6 Hz, 2H), 7.35 (dd, J = 8.8, 2.4 Hz, 1H), 7.30 (s, 1H), 6.96-6.84 (m, 2H), 6.61 (t, J = 8.8 Hz, 2H), 6.33 (dd, J = 17.0, 10.1 Hz, 1H), 6.18 (dd, J = 17.0, 2.1 Hz, 1H), 5.68 (dd, J = 10.0, 2.2 Hz, 1H), 5.04- 4.92 (m, 1H), 4.58 (t, J = 9.1 Hz, 1H), 4.52 (dd, J = 9.4, 3.8 Hz, 1H), 4.38-4.31 (m, 4H), 4.06 (t, J = 7.2 Hz, 2H), 3.70 (t, J = 6.7 Hz, 2H), 3.23-3.16 (m, 1H), 3.10-3.03 (m, 4H), 2.48 (m, 4H). |
| 196 | 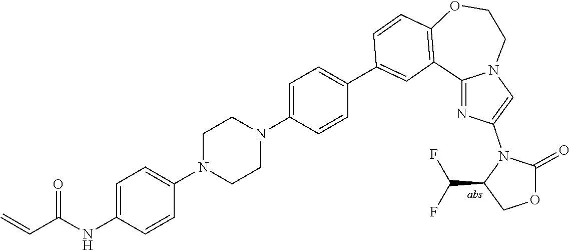 [SMILES: C=CC(=O)Nc1ccc(N2CCN(c3ccc(-c4ccc5c(c4)-c4nc(N6C(=O)OC[C@H]6C(F)F)cn4CCO5)cc3)CC2)cc1] | ¹H NMR (400 MHz, DMSO-d₆) δ 9.91 (s, 1H), 8.45 (d, J = 2.5 Hz, 1H), 7.58-7.44 (m, 5H), 7.35 (s, 1H), 7.06 (dd, J = 10.2, 8.7 Hz, 3H), 6.99-6.92 (m, 2H), 6.66 (m, 1H), 6.37 (dd, J = 17.0, 10.1 Hz, 1H), 6.17 (dd, J = 17.0, 2.1 Hz, 1H), 5.66 (dd, J = 10.1, 2.1 Hz, 1H), 5.15-4.92 (m, 1H), 4.59 (t, J = 9.1 Hz, 1H), 4.54 (dd, J = 9.3, 3.7 Hz, 1H), 4.43 (s, 4H), 3.40 (m, 4H), 3.21 (m, 4H). |
| 197 | 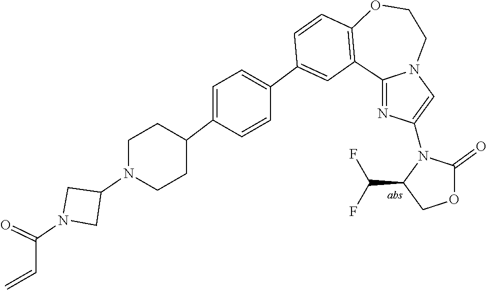 [SMILES: C=CC(=O)N1CC(N2CCC(c3ccc(-c4ccc5c(c4)-c4nc(N6C(=O)OC[C@H]6C(F)F)cn4CCO5)cc3)CC2)C1] | ¹H NMR (400 MHz, DMSO-d₆) δ 8.48 (d, J = 2.4 Hz, 1H), 7.58- 7.48 (m, 3H), 7.37-7.29 (m, 3H), 7.07 (d, J = 8.5 Hz, 1H), 6.65 (m, 1H), 6.28 (dd, J = 17.0, 10.3 Hz, 1H), 6.06 (dd, J = 17.0, 2.3 Hz, 1H), 5.63 (dd, J = 10.3, 2.3 Hz, 1H), 5.08-4.96 (m, 1H), 4.63-4.49 (m, 2H), 4.44 (s, 4H), 4.21 (t, J = 8.1 Hz, 1H), 4.00 (dd, J = 8.9, 5.1 Hz, 1H), 3.92 (dd, J = 10.3, 7.3 Hz, 1H), 3.71 (dd, J = 10.3, 5.2 Hz, 1H), 3.13-3.11 (m, 1H), 2.89 (m, 1H), 2.53 (m, 2H), 2.00-1.86 (m, 2H), 1.82-1.73 (m, 2H), 1.65-1.63 (m, 2H). |
| 199 | 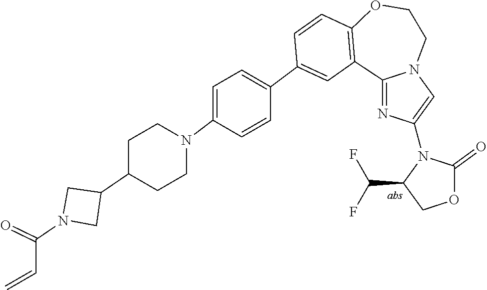 [SMILES: C=CC(=O)N1CC(C2CCN(c3ccc(-c4ccc5c(c4)-c4nc(N6C(=O)OC[C@H]6C(F)F)cn4CCO5)cc3)CC2)C1] | ¹H NMR (400 MHz, DMSO-d₆) δ 8.47 (d, J = 2.4 Hz, 1H), 7.49 (dd, J = 8.6, 2.5 Hz, 3H), 7.38 (s, 1H), 7.07 (d, J = 8.5 Hz, 1H), 7.05- 7.00 (m, 2H), 6.85 - 6.54 (m, 1H), 6.31 (dd, J = 17.0, 10.3 Hz, 1H), 6.08 (dd, J = 17.0, 2.3 Hz, 1H), 5.65 (dd, J = 10.3, 2.3 Hz, 1H), 5.12- 5.00 (m, 1H), 4.66-4.54 (m, 2H), 4.47 (s, 4H), 4.25 (t, J = 8.5 Hz, 1H), 3.99-3.92 (m, 2H), 3.78 (d, J = 12.5 Hz, 2H), 3.67 (dd, J = 10.1, 5.8 Hz, 1H), 3.29 (m, 1H), 2.69 (dd, J = 13.1, 10.5 Hz, 2H), 2.38 (m, 1H), 1.76-1.60 (m, 2H), 1.28-1.10 (m, 2H). |
| 206 | 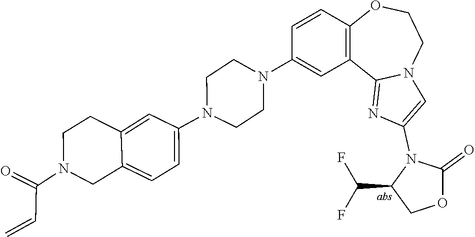 [SMILES: C=CC(=O)N1CCc2cc(N3CCN(c4ccc5c(c4)-c4nc(N6C(=O)OC[C@H]6C(F)F)cn4CCO5)CC3)ccc2C1] | ¹H NMR (400 MHz, DMSO-d₆) δ 7.83 (d, J = 3.0 Hz, 1H), 7.35 (s, 1H), 7.09-6.99 (m, 2H), 6.95 (d, J = 8.9 Hz, 1H), 6.90-6.55 (m, 4H), 6.13 (dd, J = 16.7, 2.4 Hz, 1H), 5.70 (dd, J = 10.4, 2.4 Hz, 1H), 5.09-4.97 (m, 1H), 4.64 (dd, J = 16.7, 7.5 Hz, 2H), 4.59-4.55 (m, 2H), 4.43-4.37 (m, 4H), 3.78-3.69 (m, 2H), 3.27-3.20 (m, 8H), 2.84-2.74 (m, 2H). |
| 207 | 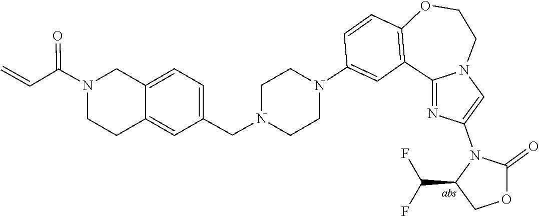 [SMILES: C=CC(=O)N1CCc2cc(CN3CCN(c4ccc5c(c4)-c4nc(N6C(=O)OC[C@H]6C(F)F)cn4CCO5)CC3)ccc2C1] | ¹H NMR (400 MHz, DMSO-d₆) δ 7.72 (d, J = 2.7 Hz, 1H), 7.29 (s, 1H), 7.14-7.08 (m, 3H), 6.93-6.79 (m, 3H), 6.75-6.47 (m, 1H), 6.10 (dd, J = 16.7, 2.4 Hz, 1H), 5.67 (dd, J = 10.4, 2.4 Hz, 1H), 5.03- 4.91 (m, 1H), 4.72 (s, 1H), 4.62 (s, 1H), 4.59-4.49 (m, 2H), 4.39- 4.27 (m, 4H), 3.73-3.71 (m, 2H), 3.44 (s, 2H), 3.26 (s, 2H), 3.04 (s, 4H), 2.84-2.73 (m, 2H), 2.49 (m, 2H). |
| 217 | 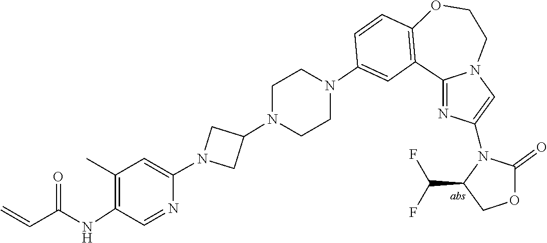 [SMILES: C=CC(=O)Nc1cnc(N2CC(N3CCN(c4ccc5c(c4)-c4nc(N6C(=O)OC[C@H]6C(F)F)cn4CCO5)CC3)C2)cc1C] | ¹H NMR (400 MHz, DMSO-d₆) δ 9.43 (s, 1H), 7.95 (s, 1H), 7.77 (d, J = 2.9 Hz, 1H), 7.34 (s, 1H), 6.96 (dd, J = 8.9, 2.9 Hz, 1H), 6.92 (d, J = 8.9 Hz, 1H), 6.83-6.51 (m, 1H), 6.44 (dd, J = 17.1, 10.2 Hz, 1H), 6.29 (s, 1H), 6.20 (dd, J = 17.1, 2.1 Hz, 1H), 5.72 (dd, J = 10.2, 2.1 Hz, 1H), 5.09-4.95 (m, 1H), 4.62 (t, J = 9.1 Hz, 1H), 4.56 (dd, J = 9.4, 3.8 Hz, 1H), 4.42-4.35 (m, 4H), 4.00 (dd, J = 8.3, 6.8 Hz, 2H), 3.77 (dd, J = 8.3, 5.2 Hz, 2H), 3.29 (m, 1H), 3.15-3.07 (m, 4H), 2.53 (m, 4H), 2.10 (s, 3H). |
| 275A | 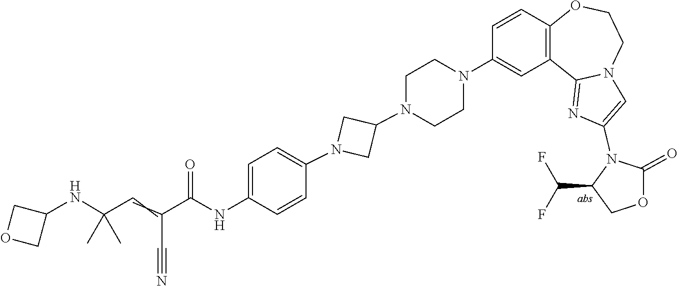 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)NC1COC1] | ¹H NMR (400 MHz, DMSO-d₆) δ 12.28 (s, 1H), 7.77 (d, J = 2.8 Hz, 1H), 7.60 (s, 1H), 7.51-7.44 (m, 2H), 7.34 (s, 1H), 6.99-6.88 (m, 2H), 6.83-6.52 (m, 1H), 6.48-6.40 (m, 2H), 5.07-4.87 (m, 4H), 4.81-4.71 (m, 1H), 4.65-4.52 (m, 2H), 4.42-4.33 (m, 4H), 3.93 (t, J = 7.0 Hz, 2H), 3.61 (dd, J = 7.4, 5.4 Hz, 2H), 3.28 (m, 1H), 3.11 (m, 4H), 2.58-2.51 (m, 4H), 1.25 (s, 6H). |
| 275B | 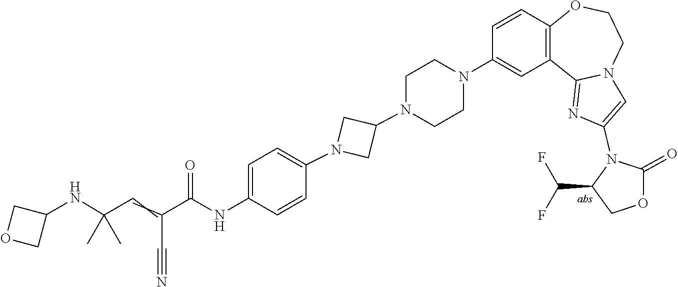 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)NC1COC1] | ¹H NMR (400 MHz, DMSO-d₆) δ 10.77 (s, 1H), 9.13 (s, 1H), 8.30 (s, 1H), 7.77 (d, J = 2.9 Hz, 1H), 7.55-7.49 (m, 2H), 7.34 (s, 1H), 6.96 (dd, J = 9.0, 2.9 Hz, 1H), 6.92 (d, J = 8.8 Hz, 1H), 6.83-6.51 (m, 1H), 6.49-6.44 (m, 2H), 5.14-4.97 (m, 4H), 4.94 (dd, J = 7.4, 5.6 Hz, 2H), 4.62 (t, J = 9.1 Hz, 1H), 4.56 (dd, J = 9.4, 3.7 Hz, 1H), 4.42-4.35 (m, 4H), 3.95 (t, J = 7.1 Hz, 2H), 3.63 (t, J = 6.5 Hz, 2H), 3.39-3.33 (m, 1H), 3.11 (m, 4H), 2.53 (m, 4H), 1.50 (s, 6H). |
| 279 | 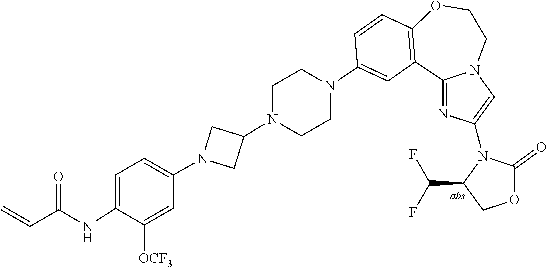 [SMILES: C=CC(=O)Nc1ccc(N2CC(N3CCN(c4ccc5c(c4)-c4nc(N6C(=O)OC[C@H]6C(F)F)cn4CCO5)CC3)C2)cc1OC(F)(F)F] | ¹H NMR (400 MHz, DMSO-d₆) δ 9.60 (s, 1H), 7.77 (d, J = 2.9 Hz, 1H), 7.52 (d, J = 8.8 Hz, 1H), 7.34 (s, 1H), 6.98-6.90 (m, 2H), 6.54 (m, 1H), 6.54-6.43 (m, 2H), 6.37 (t, J = 2.0 Hz, 1H), 6.20 (dd, J = 17.0, 2.1 Hz, 1H), 5.71 (dd, J = 10.2, 2.1 Hz, 1H), 5.08-4.95 (m, 1H), 4.64-4.54 (m, 2H), 4.41-4.36 (m, 4H), 3.97 (t, J = 7.2 Hz, 2H), 3.68 (dd, J = 7.7, 5.2 Hz, 2H), 3.41-3.38 (m, 1H), 3.14-3.08 (m, 4H), 2.53 (m, 4H). |
| 280 | 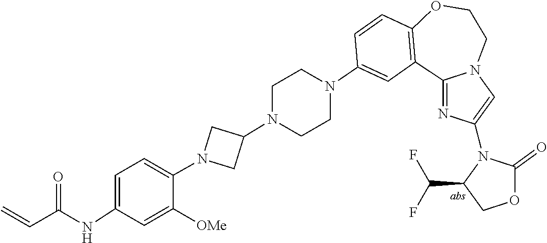 [SMILES: C=CC(=O)Nc1ccc(N2CC(N3CCN(c4ccc5c(c4)-c4nc(N6C(=O)OC[C@H]6C(F)F)cn4CCO5)CC3)C2)c(OC)c1] | ¹H NMR (400 MHz, DMSO-d₆) δ 9.87 (s, 1H), 7.73 (d, J = 2.9 Hz, 1H), 7.32-7.23 (m, 2H), 7.06 (dd, J = 8.5, 2.2 Hz, 1H), 6.96- 6.84 (m, 2H), 6.63 (m, 1H), 6.40-6.29 (m, 2H), 6.15 (dd, J = 17.0, 2.1 Hz, 1H), 5.65 (dd, J = 10.1, 2.2 Hz, 1H), 5.04-4.92 (m, 1H), 4.63-4.49 (m, 2H), 4.39-4.30 (m, 4H), 3.91 (t, J = 7.1 Hz, 2H), 3.67 (s, 3H), 3.53 (t, J = 6.8 Hz, 2H), 3.17-3.14 (m, 1H), 3.06 (s, 4H), 2.47 (m, 4H). |
| 324A | 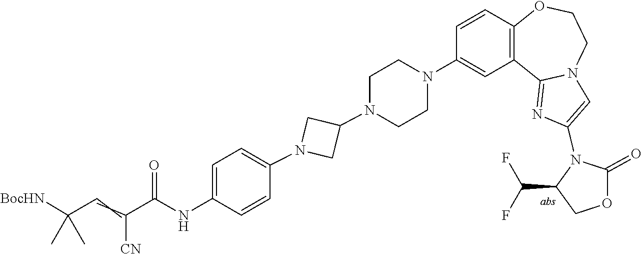 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)NC(=O)OC(C)(C)C] | ¹H NMR (400 MHz, DMSO-d₆) δ 11.65 (s, 1H), 9.37 (s, 1H), 7.77 (d, J = 2.9 Hz, 1H), 7.73 (d, J = 1.6 Hz, 1H), 7.46 (d, J = 8.8 Hz, 2H), 7.34 (s, 1H), 6.96 (dd, J = 8.9, 2.9 Hz, 1H), 6.91 (d, J = 8.9 Hz, 1H), 6.81-6.53 (m, 1H), 6.45 (d, J = 8.8 Hz, 2H), 4.62 (t, J = 9.1 Hz, 1H), 4.99 (m, 1H), 4.56 (m, 2H), 4.42-4.36 (m, 4H), 3.94 (t, J = 7.1 Hz, 2H), 3.64-3.60 (m, 2H), 3.32 (m, 1H), 3.11 (m, 4H), 2.51 (m, 4H), 1.55 (s, 9H), 1.51 (s, 6H). |
| 324B | 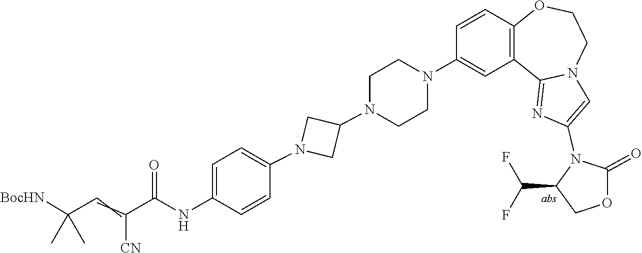 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)NC(=O)OC(C)(C)C] | ¹H NMR (400 MHz, DMSO-d₆) δ 9.85 (s, 1H), 7.77 (d, J = 2.9 Hz, 1H), 7.50 (s, 1H), 7.47-7.32 (m, 4H), 6.96 (dd, J = 9.0, 2.9 Hz, 1H), 6.91 (d, J = 8.9 Hz, 1H), 6.83-6.52 (m, 1H), 6.45-6.41 (m, 2H), 5.08-4.96 (m, 1H), 4.64-4.55 (m, 2H), 4.42-4.39 (m, 2H), 4.37 (d, J = 5.1 Hz, 2H), 3.93 (t, J = 7.0 Hz, 2H), 3.65- 3.58 (m, 2H), 3.33-3.27 (m, 1H), 3.11 (m, 4H), 2.53 (m, 4H), 1.43 (s, 6H), 1.39 (s, 9H). |
| 278 | 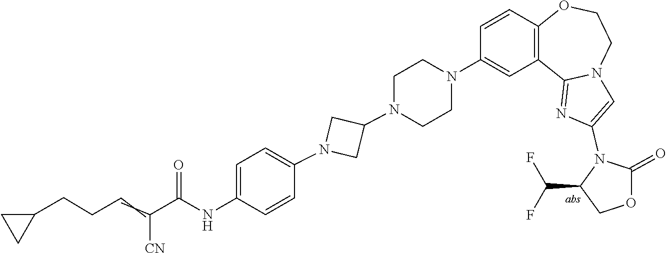 [SMILES: N#CC(=CCCC1CC1)C(=O)Nc1ccc(N2CC(N3CCN(c4ccc5c(c4)-c4nc(N6C(=O)OC[C@H]6C(F)F)cn4CCO5)CC3)C2)cc1] | ¹H NMR (400 MHz, DMSO-d₆) δ 9.75 (s, 1H), 7.77 (d, J = 2.9 Hz, 1H), 7.38 (d, J = 8.8 Hz, 2H), 7.34 (s, 1H), 7.00 (d, J = 11.0 Hz, 1H), 6.96 (dd, J = 8.9, 2.9 Hz, 1H), 6.91 (d, J = 8.8 Hz, 1H), 6.81-6.53 (m, 1H), 6.45-6.38 (m, 2H), 5.08-4.95 (m, 1H), 4.62 (t, J = 9.1 Hz, 1H), 4.56 (dd, J = 9.4, 3.8 Hz, 1H), 4.42-4.36 (m, 4H), 3.93 (t, J = 7.0 Hz, 2H), 3.60 (dd, J = 7.3, 5.6 Hz, 2H), 3.32 (m, 1H), 3.11 (m, 4H), 2.52 (m, 4H), 2.00-1.87 (m, 1H), 1.30-1.21 (m, 2H), 1.02- 0.94 (m, 2H). |
| 335 | 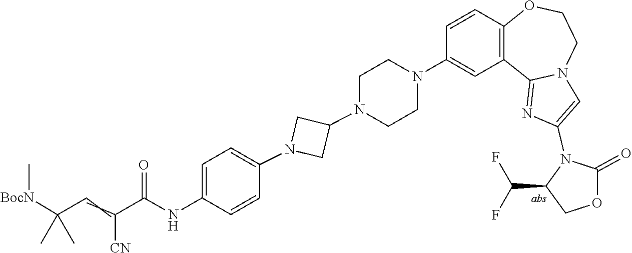 [SMILES: CN(C(=O)OC(C)(C)C)C(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1] | ¹H NMR (400 MHz, DMSO-d₆) δ 9.79 (s, 1H), 7.77 (d, J = 2.9 Hz, 1H), 7.73 (s, 1H), 7.39 (d, J = 8.8 Hz, 2H), 7.34 (s, 1H), 6.98-6.89 (m, 2H), 6.83-6.52 (m, 1H), 6.46-6.39 (m, 2H), 5.08-4.95 (m, 1H), 4.65-4.53 (m, 2H), 4.42-4.35 (m, 4H), 3.93 (t, J = 7.1 Hz, 2H), 3.62 (dd, J = 7.5, 5.4 Hz, 2H), 3.40-3.35 (m, 1H), 3.11 (s, 4H), 2.90 (s, 3H), 2.56-2.51 (m, 4H), 1.48 (s, 6H), 1.36 (s, 9H). |

### Biological Example 1. Assay for In Vitro Inhibition of PI3Kα, PI3Kβ, PI3Kγ, and PI3Kδ Kinase Activity by Compounds of the Present Disclosure

The experiment was performed using the ADP-Glo Kinase Assay kit according to the instructions. The following buffer solution was prepared: 50 mM HEPES, pH 7.5, 3 mM MgCl2, 1 mM EGTA, 100 mM NaCl, 0.03% CHAPS, 2 mM DTT. Test compound samples were dissolved in DMSO, diluted 3-fold at a starting concentration, e.g., 10 μM, and added to the screening system. A DMSO control and a kinase-free control were set simultaneously. The optimal concentrations of PI3Kα, PI3Kδ, PI3Kβ, and PI3Kγ enzymes, substrate (PIP2), and ATP were prepared with buffer. The enzyme reaction system contained: buffer, 25 μM ATP, kinase substrate (PIP2, 50 μg/mL), and kinases PI3Kα (0.15 μg/mL), PI3Kδ (1.2 μg/mL), PI3Kβ (0.3 μg/mL), PI3Kγ (2.5 μg/mL), and the like. The reaction system was reacted at room temperature for 1 h. The reaction was terminated by adding a termination reagent (ADP-Glo reagent, 5 μL), and the ADP content in the system was detected using a detection reagent (Kinase Detection Reagent, 10 μL). Signal data were collected using an Envision instrument. The inhibition rate was calculated according to the following equation: % inhibition=(DMSO control signal value−sample signal value)/(DMSO control signal value−kinase-free control signal value). The IC₅₀ values were obtained by fitting a curve using the equation: Y=Bottom+(Top−Bottom)/(1+(IC₅₀/X)\hat{ }HillSlope). The results are shown in Table 3 below.

TABLE 3
Inhibitory activity of the compounds of the present
disclosure against PI3K kinases (IC₅₀, nM)

| Compound | PI3Kα | PI3Kβ | PI3Kγ | PI3Kδ | Compound | PI3Kα | PI3Kβ | PI3Kγ | PI3Kδ |
|---|---|---|---|---|---|---|---|---|---|
| 1 | 5.4 | 317 | 21 | <8 | 2 | 7.3 | 513 | 44 | 20 |
| 4 | 7.4 | 315 | 98 | 43 | 5 | 50 | 314 | 1205 | 117 |
| 17 | 11 | 314 | 317 | 92 | 27 | 0.21 | 2923 | 26 | 76 |
| 29 | 1.18 | 1256 | 38 | 49 | 95 | 0.16 | 1092 | 42 | 103 |
| 96 | 1.33 | 3163 | 223 | 475 | | | | | |

### Biological Example 2. Assay for In Vitro Inhibition of HCC1954 Cell Proliferation by Compounds of the Present Disclosure

HCC1954 cells (with PI3Kα H1047R mutation) were seeded into a 384-well plate (Fisher 142762) at 2000 cells per well. The next day, serially diluted compounds were added, and 144 h after the addition, CellTiter-Lumi (Beyotime C0068XL) was added to measure the ATP content in the cells. The growth of the cells was evaluated, and the IC₅₀ for the inhibition of cell growth by the compounds was calculated. The results are shown in Table 4 below.

TABLE 4
Inhibitory activity of the compounds of the present disclosure against HCC1954 cells (IC₅₀, nM)

| Compound | IC₅₀ | Compound | IC₅₀ | Compound | IC₅₀ | Compound | IC₅₀ | Compound | IC₅₀ |
|---|---|---|---|---|---|---|---|---|---|
| 1 | 103 | 2 | 97 | 3 | 282 | 4 | 65 | 5 | 670 |
| 6 | 663 | 7 | 925 | 8 | >1000 | 9 | >1000 | 10 | >1000 |
| 11 | 298 | 12 | 399 | 13 | 862 | 14 | >1000 | 15 | 128 |
| 16 | 63 | 17 | 81 | 18 | 68 | 19 | >1000 | 20 | 438 |
| 21 | 251 | 22 | >1000 | 23 | >1000 | 24 | >1000 | 25 | >1000 |
| 26 | 248 | 27 | 9 | 28 | 180 | 29 | 75 | 37 | 750 |
| 39 | 68 | 40 | 162 | 41 | 191 | 43 | 184 | 44 | 644 |
| 45 | >1000 | 46 | 306 | 59 | 872 | 60 | 174 | 64 | 314 |
| 85 | 303 | 86 | 126 | 87 | 318 | 88 | 212 | 89 | 230 |
| 90 | 225 | 91 | 154 | 92 | 200 | 93 | 551 | 94 | 300 |
| 95 | 68 | 96 | 26 | 97 | 38 | 98 | 59 | 99 | 53 |
| 100 | 66 | 101 | 83 | 102 | 232 | 103 | 23 | 104 | 65 |
| 105 | 620 | 106 | 6 | 121 | 3 | 122 | 7 | 123 | 5.3 |
| 124 | 9 | 129 | 6.9 | 130 | 70 | 131 | 84 | 132 | 45 |
| 133 | 4.3 | 134 | 2.7 | 135 | 16 | 136 | 71 | 137 | 0.6 |
| 138 | 109 | 139 | 6.7 | 140 | 6 | 141 | 6.7 | 142 | 8.3 |
| 143 | 2.2 | 144 | 55 | 145 | 0.6 | 146 | 75 | 147 | 29 |
| 148 | 10 | 149 | 115 | 150 | 121 | 151 | 5.6 | 152 | 49 |
| 153 | 10 | 154 | 54 | 155 | 44 | 156 | 49 | 157 | 228 |
| 158 | 249 | 159 | 28 | 160 | 47 | 161 | 2.9 | 162 | 3.4 |
| 163 | 6.6 | 164 | 53 | 165 | 30 | 166 | 9.4 | 167 | 544 |
| 107 | 131 | 111 | 2.5 | 168 | 4.9 | 169 | 1 | 170 | 77 |
| 171 | 14 | 172 | 3.6 | 173 | 4 | 174 | 166 | 175 | 1.5 |
| 176 | 4.9 | 177 | 2.2 | 178 | ++++ | 179 | +++ | 180 | +++ |
| 181 | +++ | 182 | +++ | 183 | 219 | 184 | ++++ | 185 | ++++ |
| 186 | 32 | 187 | ++++ | 188 | +++ | 189 | +++ | 190 | +++ |
| 191 | +++ | 192 | +++ | 193 | ++++ | 194 | 1.2 | 195 | 4.6 |
| 196 | + | 197 | ++ | 198 | ++ | 199 | ++ | 200 | +++ |
| 201 | +++ | 202 | +++ | 203 | +++ | 204 | +++ | 205 | +++ |
| 206 | +++ | 207 | +++ | 208 | +++ | 209 | +++ | 210 | +++ |
| 211 | ++++ | 212 | ++++ | 213 | ++++ | 214 | ++++ | 215 | ++++ |
| 216 | ++++ | 217 | ++++ | 218 | ++++ | 219 | ++++ | 220 | ++++ |
| 221 | ++++ | 222 | ++++ | 223 | ++++ | 224 | ++++ | 225 | ++++ |
| 226 | ++++ | 227 | ++++ | 228 | ++++ | 229 | ++++ | 230 | ++++ |
| 231 | ++++ | 232 | ++++ | 233 | ++++ | 234 | ++++ | 235 | ++++ |
| 236 | ++++ | 237 | ++++ | 238 | ++++ | 239 | ++++ | 240 | ++++ |
| 241 | ++++ | 242 | ++++ | 243 | ++++ | 244 | ++++ | 245 | ++++ |
| 246 | ++++ | 247 | ++++ | 248 | ++++ | 249 | ++++ | 250 | ++++ |
| 251 | ++++ | 252 | ++++ | 253 | ++++ | 254 | ++++ | 255 | ++++ |
| 256 | ++++ | 257 | ++++ | 258 | ++++ | 259 | ++++ | 260 | ++++ |
| 261 | ++++ | 262 | ++++ | 263 | ++++ | 264 | ++++ | 265 | ++++ |
| 266 | ++++ | 267 | ++++ | 268 | ++++ | 269 | ++++ | 270 | ++++ |
| 271 | ++++ | 272 | ++++ | 273 | ++++ | 274 | ++++ | 275 | ++++ |
| 276 | ++++ | 277 | ++++ | 278 | 42 | 279 | 2.8 | 280 | 3.4 |
| 281 | ++++ | 282 | ++++ | 283 | ++++ | 284 | ++++ | 285 | ++++ |
| 286 | ++++ | 287 | ++++ | 288 | ++++ | 289 | ++++ | 290 | ++++ |
| 291 | ++++ | 292 | ++++ | 293 | ++++ | 294 | ++++ | 295 | ++++ |
| 296 | ++++ | 297 | ++++ | 298 | ++++ | 299 | ++++ | 300 | ++++ |
| 301 | ++++ | 302 | ++++ | 303 | ++++ | 304 | ++++ | 305 | ++++ |
| 306 | ++++ | 307 | ++++ | 308 | ++++ | 309 | ++++ | 310 | ++++ |
| 311 | ++++ | 312 | ++++ | 313 | ++++ | 314 | ++++ | 315 | ++++ |
| 316 | ++++ | 317 | ++++ | 318 | ++++ | 319 | ++++ | 320 | ++++ |
| 321 | ++++ | 322 | ++++ | 323 | ++++ | 324A | 0.1 | 324B | 6.2 |
| 325 | ++++ | 326 | ++++ | 327 | ++++ | 328 | ++++ | 329 | ++++ |
| 330 | ++++ | 331 | ++++ | 332 | ++++ | 333 | ++++ | 334 | ++++ |
| 335 | ++++ | 336 | ++++ | 337 | ++++ | 338 | ++++ | 339 | ++++ |
| 340 | ++++ | 341 | ++++ | 342 | ++++ | 343 | ++++ | 344 | ++++ |
| 345 | ++++ | 346 | ++++ | 347 | ++++ | 348 | ++++ | 349 | ++++ |
| 350 | ++++ | 351 | ++++ | 352 | ++++ | 353 | ++++ | 354 | ++++ |
| 355 | ++++ | 356 | ++++ | 357 | ++++ | 358 | ++++ | 359 | ++++ |
| 360 | ++++ | 361 | ++++ | 362 | ++++ | 363 | ++++ | 364 | ++++ |
| 365 | ++++ | 366 | ++++ | 367 | ++++ | 368 | ++++ | 369 | ++++ |
| 370 | ++++ | 371 | ++++ | 372 | ++++ | 373 | ++++ | 374 | ++++ |
| 375 | ++++ | 376 | ++++ | 377 | ++++ | 378 | ++++ | 379 | ++++ |
| 380 | ++++ | 381 | ++++ | 382 | ++++ | 383 | ++++ | 384 | ++++ |
| 385 | ++++ | 386 | ++++ | 387 | ++++ | 388 | ++++ | 389 | ++++ |
| 390 | ++++ | 391 | ++++ | 392 | ++++ | 393 | ++++ | 394 | ++++ |
| 395 | ++++ | CNX1351 | 1190 | Alpelisib | 1614 | GDC0077 | 82 | | |

<50 nM: ++++
50 to 100 nM: +++
100 to 1000 nM: ++
>1000 nM: +

### Biological Example 3. Assay for In Vitro Inhibition of MCF-7 Cell Proliferation by Compounds of the Present Disclosure

MCF-7 cells (with PI3Kα E545K mutation) were seeded into a 384-well plate (Fisher 142762) at 2000 cells per well. The next day, serially diluted compounds were added, and 144 h after the addition, CellTiter-Lumi (Beyotime C0068XL) was added to measure the ATP content in the cells. The growth of the cells was evaluated, and the IC₅₀ for the inhibition of cell growth by the compounds was calculated. The results are shown in Table 5 below.

TABLE 5
Inhibitory activity of the compounds of the present disclosure against MCF-7 cells (IC₅₀, nM)

| Compound | IC₅₀ | Compound | IC₅₀ | Compound | IC₅₀ | Compound | IC₅₀ | Compound | IC₅₀ |
|---|---|---|---|---|---|---|---|---|---|
| 1 | 18 | 2 | 28 | 3 | 62 | 4 | 26 | 5 | 35 |
| 6 | 99 | 7 | 106 | 8 | 63 | 9 | 75 | 10 | 146 |
| 11 | 58 | 12 | 49 | 13 | 40 | 14 | 264 | 15 | 14 |
| 16 | 7 | 17 | 16 | 18 | 25 | 19 | 217 | 20 | 69 |
| 21 | 23 | 22 | 143 | 23 | 186 | 24 | 1119 | 25 | 390 |
| 26 | 83 | 27 | 0.36 | 28 | 18 | 29 | 13 | 37 | 75 |
| 39 | 11 | 40 | 34 | 41 | 40 | 43 | 19 | 44 | 151 |
| 45 | 244 | 46 | 74 | 59 | 197 | 60 | 13 | 64 | 33 |
| 85 | 88 | 86 | 17 | 87 | 37 | 88 | 32 | 89 | 41 |
| 90 | 12 | 91 | 11 | 92 | 20 | 93 | 76 | 94 | 76 |
| 95 | 38 | 96 | 2.5 | 97 | 3.7 | 98 | 6.7 | 99 | 11 |
| 100 | 22 | 101 | 38 | 102 | 23 | 103 | 2 | 104 | 4.6 |
| 105 | 23 | 106 | 0.14 | 121 | 0.2 | 122 | 0.4 | 123 | 0.9 |
| 124 | 2.7 | 129 | 0.3 | 130 | 14 | 131 | 15 | 132 | 3.3 |
| 133 | 0.4 | 134 | 0.2 | 135 | 1.5 | 136 | 29 | 137 | 0.1 |
| 138 | 35 | 139 | 0.6 | 140 | 0.3 | 141 | 0.6 | 142 | 0.5 |
| 143 | 0.1 | 144 | 22 | 145 | 0.1 | 146 | 6.6 | 147 | 1.5 |
| 148 | 0.2 | 149 | 16 | 150 | 35 | 151 | 0.3 | 152 | 9.7 |
| 153 | 0.8 | 154 | 4.9 | 155 | 5.1 | 156 | 19 | 157 | 24 |
| 158 | 321 | 159 | 1.8 | 160 | 3.5 | 161 | 0.5 | 162 | 0.4 |
| 163 | 0.5 | 164 | 3 | 165 | 3.4 | 166 | 1.2 | 167 | 219 |
| 107 | 16 | 111 | 0.1 | 168 | 6.9 | 169 | 0.1 | 170 | 17 |
| 171 | 4.5 | 172 | 0.2 | 173 | 0.3 | 174 | 26 | 175 | 0.1 |
| 176 | 0.2 | 177 | 0.1 | 178 | ++++ | 179 | +++ | 180 | +++ |
| 181 | ++++ | 182 | +++ | 183 | 21 | 184 | ++++ | 185 | ++++ |
| 186 | 4.3 | 187 | ++++ | 188 | ++++ | 189 | ++++ | 190 | ++++ |
| 191 | ++++ | 192 | ++++ | 193 | ++++ | 194 | 0.1 | 195 | 0.3 |
| 196 | + | 197 | ++++ | 198 | +++ | 199 | ++++ | 200 | ++++ |
| 201 | ++++ | 202 | ++++ | 203 | ++++ | 204 | ++++ | 205 | ++++ |
| 206 | ++++ | 207 | ++++ | 208 | ++++ | 209 | ++++ | 210 | ++++ |
| 211 | ++++ | 212 | ++++ | 213 | ++++ | 214 | ++++ | 215 | ++++ |
| 216 | ++++ | 217 | ++++ | 218 | ++++ | 219 | ++++ | 220 | ++++ |
| 221 | ++++ | 222 | ++++ | 223 | ++++ | 224 | ++++ | 225 | ++++ |
| 226 | ++++ | 227 | ++++ | 228 | ++++ | 229 | ++++ | 230 | ++++ |
| 231 | ++++ | 232 | ++++ | 233 | ++++ | 234 | ++++ | 235 | ++++ |
| 236 | ++++ | 237 | ++++ | 238 | ++++ | 239 | ++++ | 240 | ++++ |
| 241 | ++++ | 242 | ++++ | 243 | ++++ | 244 | ++++ | 245 | ++++ |
| 246 | ++++ | 247 | ++++ | 248 | ++++ | 249 | ++++ | 250 | ++++ |
| 251 | ++++ | 252 | ++++ | 253 | ++++ | 254 | ++++ | 255 | ++++ |
| 256 | ++++ | 257 | ++++ | 258 | ++++ | 259 | ++++ | 260 | ++++ |
| 261 | ++++ | 262 | ++++ | 263 | ++++ | 264 | ++++ | 265 | ++++ |
| 266 | ++++ | 267 | ++++ | 268 | ++++ | 269 | ++++ | 270 | ++++ |
| 271 | ++++ | 272 | ++++ | 273 | ++++ | 274 | ++++ | 275 | ++++ |
| 276 | ++++ | 277 | ++++ | 278 | 16 | 279 | 0.1 | 280 | 0.2 |
| 281 | ++++ | 282 | ++++ | 283 | ++++ | 284 | ++++ | 285 | ++++ |
| 286 | ++++ | 287 | ++++ | 288 | ++++ | 289 | ++++ | 290 | ++++ |
| 291 | ++++ | 292 | ++++ | 293 | ++++ | 294 | ++++ | 295 | ++++ |
| 296 | ++++ | 297 | ++++ | 298 | ++++ | 299 | ++++ | 300 | ++++ |
| 301 | ++++ | 302 | ++++ | 303 | ++++ | 304 | ++++ | 305 | ++++ |
| 306 | ++++ | 307 | ++++ | 308 | ++++ | 309 | ++++ | 310 | ++++ |
| 311 | ++++ | 312 | ++++ | 313 | ++++ | 314 | ++++ | 315 | ++++ |
| 316 | ++++ | 317 | ++++ | 318 | ++++ | 319 | ++++ | 320 | ++++ |
| 321 | ++++ | 322 | ++++ | 323 | ++++ | 324A | 0.1 | 324B | 2.6 |
| 325 | ++++ | 326 | ++++ | 327 | ++++ | 328 | ++++ | 329 | ++++ |
| 330 | ++++ | 331 | ++++ | 332 | ++++ | 333 | ++++ | 334 | ++++ |
| 335 | ++++ | 336 | ++++ | 337 | ++++ | 338 | ++++ | 339 | ++++ |
| 340 | ++++ | 341 | ++++ | 342 | ++++ | 343 | ++++ | 344 | ++++ |
| 345 | ++++ | 346 | ++++ | 347 | ++++ | 348 | ++++ | 349 | ++++ |
| 350 | ++++ | 351 | ++++ | 352 | ++++ | 353 | ++++ | 354 | ++++ |
| 355 | ++++ | 356 | ++++ | 357 | ++++ | 358 | ++++ | 359 | ++++ |
| 360 | ++++ | 361 | ++++ | 362 | ++++ | 363 | ++++ | 364 | ++++ |
| 365 | ++++ | 366 | ++++ | 367 | ++++ | 368 | ++++ | 369 | ++++ |
| 370 | ++++ | 371 | ++++ | 372 | ++++ | 373 | ++++ | 374 | ++++ |
| 375 | ++++ | 376 | ++++ | 377 | ++++ | 378 | ++++ | 379 | ++++ |
| 380 | ++++ | 381 | ++++ | 382 | ++++ | 383 | ++++ | 384 | ++++ |
| 385 | ++++ | 386 | ++++ | 387 | ++++ | 388 | ++++ | 389 | ++++ |
| 390 | ++++ | 391 | ++++ | 392 | ++++ | 393 | ++++ | 394 | ++++ |
| 395 | ++++ | CNX1351 | 290 | Alpelisib | 216 | GDC0077 | 55 | | |

<50 nM: +++
50 to 100 nM: +++
100 to 1000 nM: ++
>1000 nM: +

Although specific embodiments of the present disclosure have been described above, it will be appreciated by those skilled in the art that these embodiments are merely illustrative and that many changes or modifications can be made to these embodiments without departing from the principles and spirit of the present disclosure. The protection scope of the present disclosure is therefore defined by the appended claims.

1. A compound of general formula (1) or an isomer thereof, a crystalline form thereof, a pharmaceutically acceptable salt thereof, a hydrate thereof, or a solvate thereof: 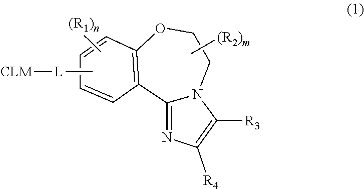
wherein in general formula (1):
CLM is a group that can covalently bind to a PI3Kα protein;
L is a group connecting CLM and a tricyclic structure;
R₁ and R₂ are independently selected from hydrogen, deuterium, alkyl, deuterated alkyl, haloalkyl, alkoxy, haloalkoxy, halogen, amino, carbamoyl, sulfydryl, nitro, hydroxy, cyano, alkenyl, alkynyl, cycloalkyl, heterocycloalkyl, —(CH₂)ₙ₁Rₐ, —(CH₂)ₙ₁ORₐ, and —(CH₂)ₙ₁NRₐR_{b}, wherein the alkyl, deuterated alkyl, haloalkyl, alkoxy, haloalkoxy, amino, carbamoyl, sulfydryl, hydroxy, alkenyl, alkynyl, cycloalkyl, or heterocycloalkyl is optionally further substituted with one or more substituents selected from deuterium, substituted or unsubstituted cycloalkylalkyl, substituted or unsubstituted cycloalkylhaloalkyl, halogen, substituted or unsubstituted cycloalkylamino, sulfydryl, oxo, nitro, cyano, hydroxy, substituted or unsubstituted alkenyl, substituted or unsubstituted alkynyl, substituted or unsubstituted cycloalkylalkoxy, substituted or unsubstituted cycloalkylhaloalkoxy, substituted or unsubstituted cycloalkylhydroxyalkyl, substituted or unsubstituted cycloalkyl, substituted or unsubstituted heterocycloalkyl, —(CH₂)ₙ₁R_{c}, —(CH₂)ₙ₁OR_{c}, and —(CH₂)ₙ₁NR_{c}R_{d};
or any two adjacent or non-adjacent R₂ form one cycloalkyl or heterocycloalkyl group, wherein the cycloalkyl or heterocycloalkyl group is optionally further substituted with one or more substituents selected from deuterium, substituted or unsubstituted alkyl, substituted or unsubstituted haloalkyl, halogen, substituted or unsubstituted amino, oxo, nitro, cyano, hydroxy, substituted or unsubstituted alkenyl, substituted or unsubstituted alkynyl, substituted or unsubstituted alkoxy, substituted or unsubstituted haloalkoxy, substituted or unsubstituted hydroxyalkyl, substituted or unsubstituted cycloalkyl, and substituted or unsubstituted heterocycloalkyl;
Rₐ, R_{b}, R_{c}, and R_{d} are each independently selected from hydrogen, deuterium, alkyl, deuterated alkyl, haloalkyl, alkoxy, hydroxyalkyl, haloalkoxy, halogen, cyano, nitro, hydroxy, amino, carbamoyl, alkenyl, alkynyl, cycloalkyl, and heterocycloalkyl, wherein the alkyl, deuterated alkyl, haloalkyl, alkoxy, hydroxyalkyl, haloalkoxy, alkenyl, alkynyl, cycloalkyl, or heterocycloalkyl is optionally further substituted with one or more substituents selected from deuterium, substituted or unsubstituted alkyl, halogen, hydroxy, substituted or unsubstituted amino, oxo, nitro, cyano, substituted or unsubstituted alkenyl, substituted or unsubstituted alkynyl, substituted or unsubstituted alkoxy, substituted or unsubstituted hydroxyalkyl, substituted or unsubstituted cycloalkyl, and substituted or unsubstituted heterocycloalkyl;
R₃ is H, halogen, (C1-C3) alkyl, or cyclopropyl;
R₄ is 4- to 6-membered heterocycloalkyl containing 1 to 2 heteroatoms independently selected from N, S, and O, phenyl, or 5- to 6-membered heteroaryl containing 1 to 4 heteroatoms independently selected from N, S, and O, wherein the 4- to 6-membered heterocycloalkyl, phenyl, or 5- to 6-membered heteroaryl is optionally further substituted with one or more substituents selected from deuterium, alkyl, deuterated alkyl, haloalkyl, alkoxy, haloalkoxy, halogen, amino, nitro, hydroxy, cyano, cycloalkyl, heterocycloalkyl, oxoheterocycloalkyl, thioheterocycloalkyl, oxo, and thio; and
n is 0, 1, 2, or 3;
m is 0, 1, 2, 3, or 4;
n1 is 0, 1, 2, or 3.

2. The compound or the isomer thereof, the crystalline form thereof, the pharmaceutically acceptable salt thereof, the hydrate thereof, or the solvate thereof according to claim 1, wherein the general formula (1) has a structure as shown in general formula (2): 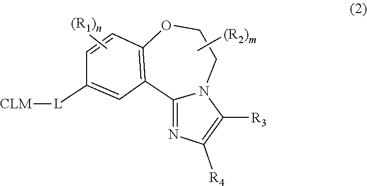
wherein CLM, L, R₁, R₂, R₃, R₄, m, and n are as defined above.

3. The compound or the isomer thereof, the crystalline form thereof, the pharmaceutically acceptable salt thereof, the hydrate thereof, or the solvate thereof according to claim 1, wherein in the general formula (1) or general formula (2), CLM is a group that can covalently bind to a cysteine residue (Cys) in the PI3Kα protein and comprises a carbon-carbon double bond or carbon-carbon triple bond.

4. The compound or the isomer thereof, the crystalline form thereof, the pharmaceutically acceptable salt thereof, the hydrate thereof, or the solvate thereof according to claim 3, wherein in the general formula (1) or general formula (2), CLM is —NHCN, —CN, 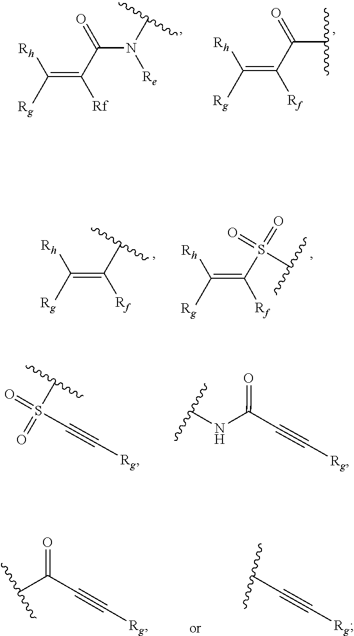
Rₑ is H, substituted or unsubstituted (C1-C6) alkyl, or substituted or unsubstituted (C3-C6) cycloalkyl;
R_{f}, R_{g}, and Rₕ are each independently H, halogen, —CN, —C(O)R, —C(O)OR, —C(O)NR₂, —C(O)N(R)OR, —(CH₂)_{w}R, —(CH₂)_{w}OR, —(CH₂)_{w}N(R)₂, substituted or unsubstituted (C1-C6) alkyl, substituted or unsubstituted (C3-C6) cycloalkyl, substituted or unsubstituted phenyl, substituted or unsubstituted 3- to 7-membered saturated or partially unsaturated heterocycloalkyl containing 1 to 2 heteroatoms independently selected from N, S, and O, or substituted or unsubstituted 5- to 6-membered heteroaryl containing 1 to 4 heteroatoms independently selected from N, S, and O;
or Rₑ and R_{f}, R_{f} and R_{g}, R_{g} and Rₕ, or Rₑ and Rₕ may form a substituted or unsubstituted 4- to 7-membered saturated or partially unsaturated ring containing 0 to 2 heteroatoms independently selected from N, S, and O, wherein two hydrogen atoms on the same carbon atom of the 4- to 7-membered saturated or partially unsaturated ring may be substituted with oxygen to form oxo;
each R is independently H, substituted or unsubstituted (C1-C6) alkyl, substituted or unsubstituted (C3-C6) cycloalkyl, substituted or unsubstituted phenyl, substituted or unsubstituted 3- to 7-membered saturated or partially unsaturated heterocycloalkyl containing 1 to 2 heteroatoms independently selected from N, S, and O, or substituted or unsubstituted 5- to 6-membered heteroaryl containing 1 to 4 heteroatoms independently selected from N, S, and O;
w is 0, 1, or 2.

5. The compound or the isomer thereof, the crystalline form thereof, the pharmaceutically acceptable salt thereof, the hydrate thereof, or the solvate thereof wherein in the general formula (1) or general formula (2), CLM is —NHCN, —CN, 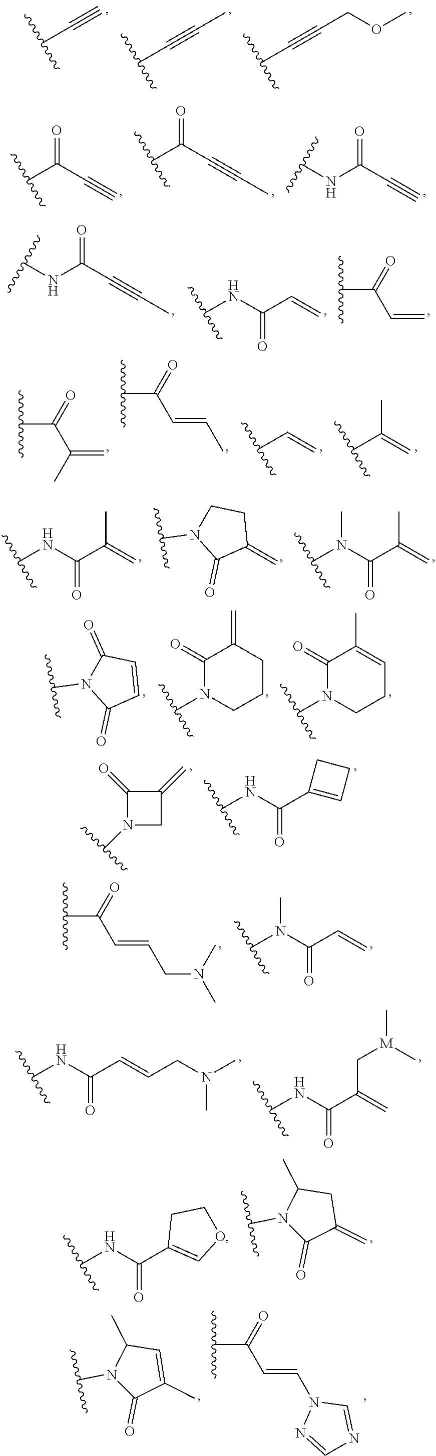 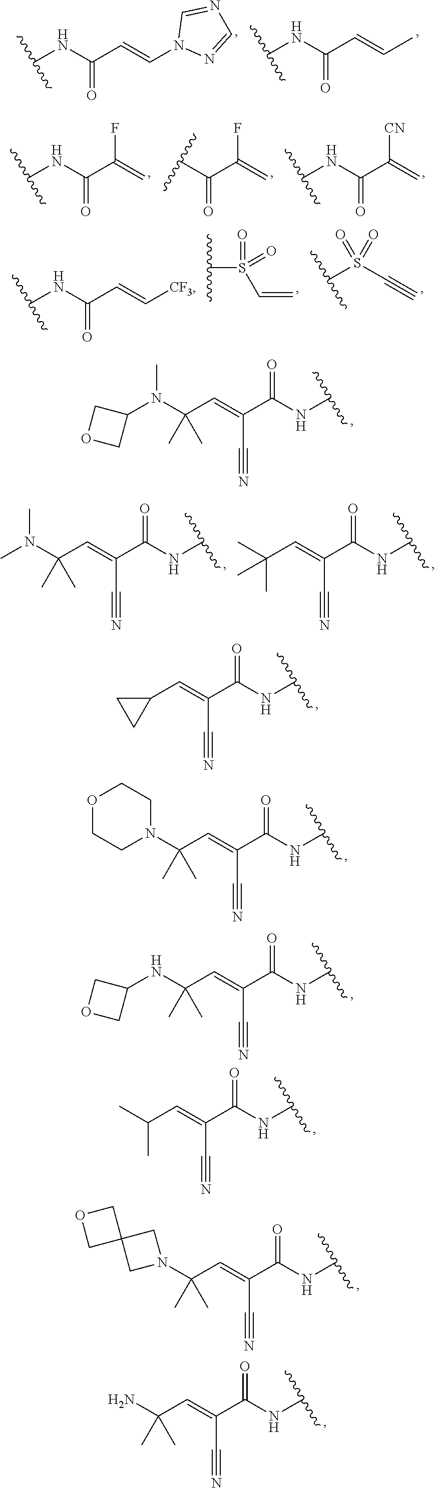 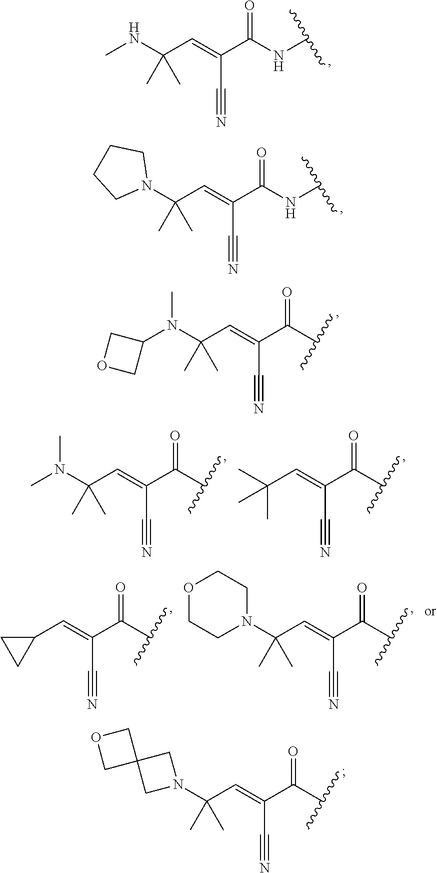
CLM is preferably —NHCN, —CN, 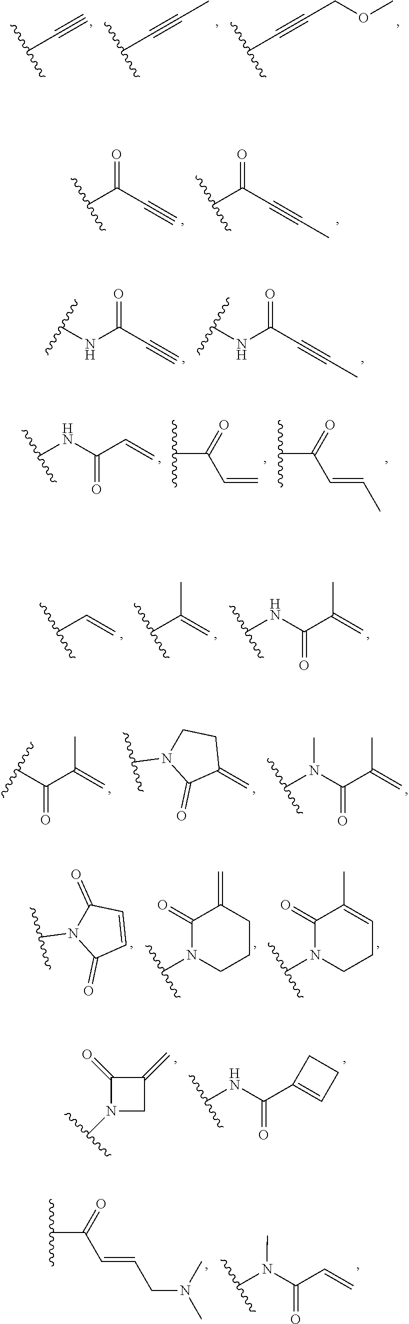 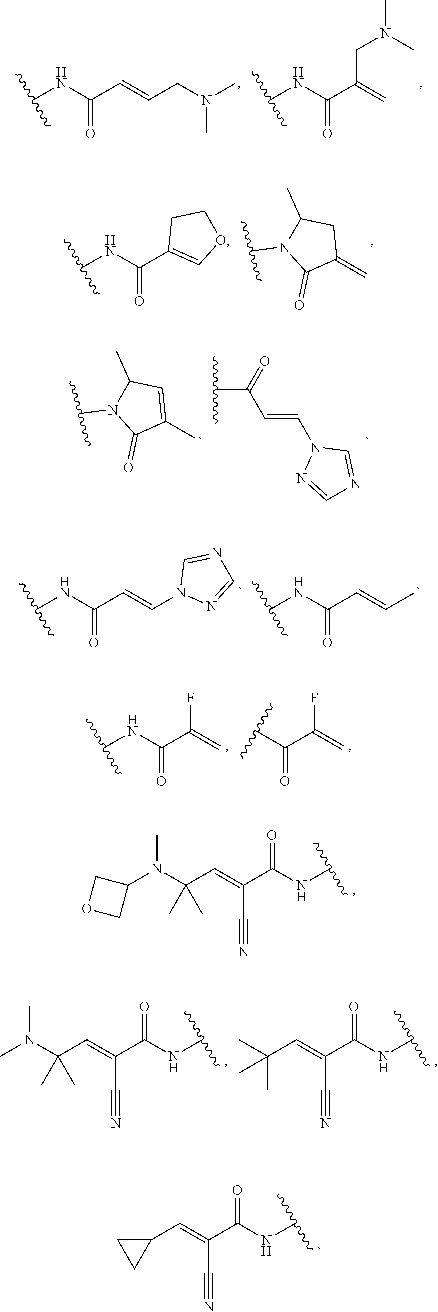 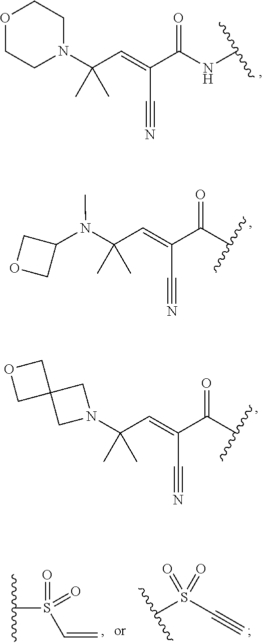
CLM is more preferably

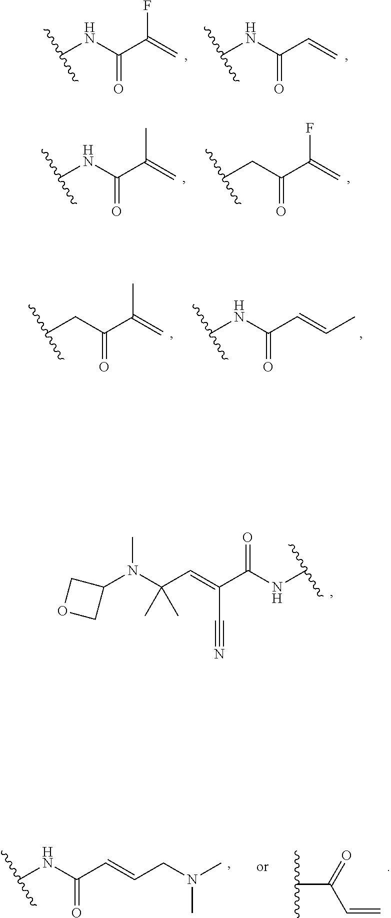

6. The compound or the isomer thereof, the crystalline form thereof, the pharmaceutically acceptable salt thereof, the hydrate thereof, or the solvate thereof according to claim 1, the general formula (1) or general formula (2), L is 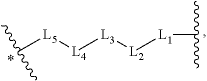
wherein L₁, L₂, L₃, L₄, and L₅ are each independently selected from a chemical bond, O, S, NH, C(═O), C(═O)NH, S(═O), S(═O)₂, (C1-C6) alkylene, —(C1-C6) alkylene-O—, (C2-C3) alkenylene, (C2-C3) alkynylene, (C3-C10) cycloalkylene, phenylene, 5- to 11-membered bridged cyclylene, 3- to 10-membered saturated or partially unsaturated heterocycloalkylene containing 1 to 4 heteroatoms independently selected from N, S, and O, 7- to 11-membered spiro heterocyclylene, 5- to 11-membered bridged heterocyclylene, and 5- to 9-membered heteroarylene, wherein the (C1-C6) alkylene, —(C1-C6) alkylene-O—, (C2-C3) alkenylene, (C2-C3) alkynylene, (C3-C10) cycloalkylene, 3- to 10-membered saturated or partially unsaturated heterocycloalkylene, phenylene, 7- to 11-membered spiro heterocyclylene, 5- to 11-membered bridged heterocyclylene, or 5- to 9-membered heteroarylene is optionally substituted with 1, 2, or 3 R_{L};
each R_{L} is independently selected from H, halogen, OH, NH₂, CN, —CONH₂, (C1-C6) alkyl, (C3-C6) cycloalkyl, (C1-C6) alkyl-C(═O)—, (C1-C6) alkoxy, (C1-C6) alkylthio, and (C1-C6) alkylamino, wherein the (C1-C6) alkyl, (C3-C6) cycloalkyl, (C1-C6) alkyl-C(═O)—, (C1-C6) alkoxy, (C1-C6) alkylthio, or (C1-C6) alkylamino is optionally substituted with 1, 2, or 3 R_{LL};
each R_{LL} is independently selected from H, halogen, (C1-C6) alkyl, OH, NH₂, MeNH—, Me₂N—, CH₃, CH₂F, CHF₂, and CF₃; wherein * denotes the connection to CLM.

7. The compound or the isomer thereof, the crystalline form thereof, the pharmaceutically acceptable salt thereof, the hydrate thereof, or the solvate thereof according to claim 6, wherein in the general formula (1) or general formula (2), each of L, L₂, L₃, L₄, and L₅ is independently a chemical bond, O, S, NH, C(═O), C(═O)NH, S(═O), S(═O)₂, CH₂,

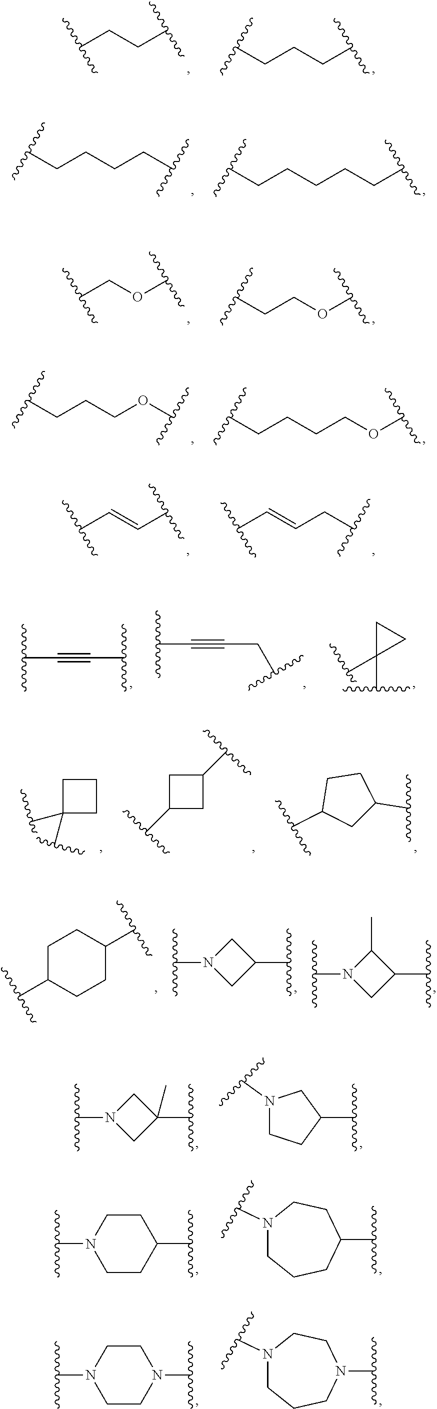

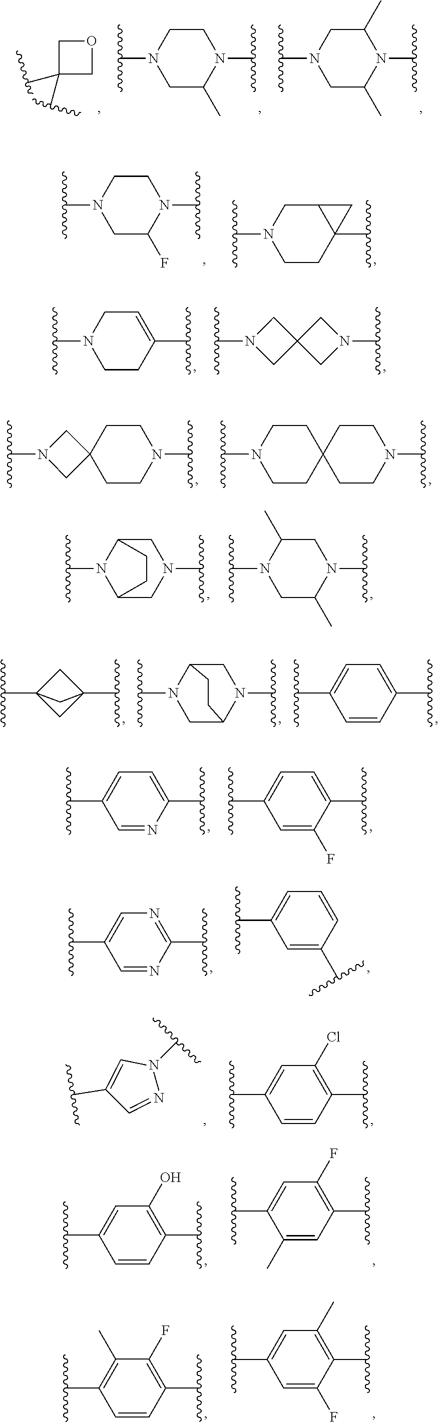

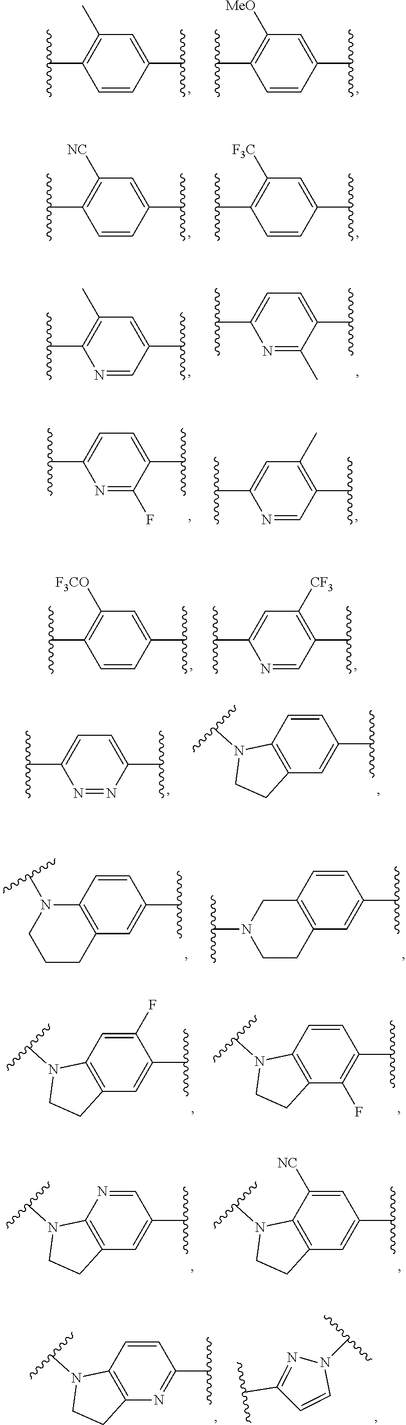

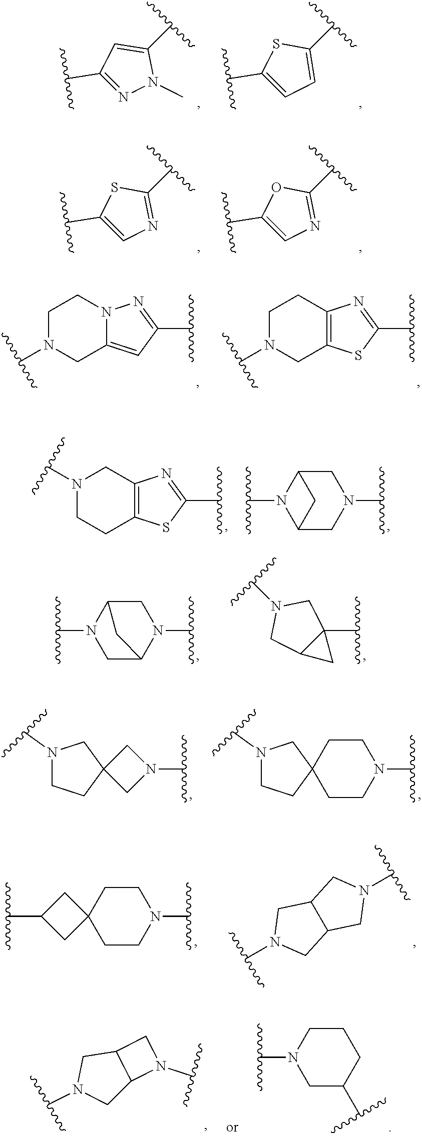

8. The compound or the isomer thereof, the crystalline form thereof, the pharmaceutically acceptable salt thereof, the hydrate thereof, or the solvate thereof according to claim 6, wherein in the general formula (1) or general formula (2),
L₁ is a chemical bond, (C2-C3) alkenylene, (C2-C3) alkynylene, (C3-C10) cycloalkylene, 3- to 10-membered saturated or partially unsaturated heterocycloalkylene containing 1 to 4 heteroatoms independently selected from N, S, and O, 7- to 11-membered spiro heterocyclylene, or 5- to 11-membered bridged heterocyclylene, wherein the (C2-C3) alkenylene, (C2-C3) alkynylene, (C3-C10) cycloalkylene, 3- to 10-membered saturated or partially unsaturated heterocycloalkylene, 7- to 11-membered spiro heterocyclylene, or 5- to 11-membered bridged heterocyclylene is optionally substituted with 1, 2, or 3 R_{L};
L₂ is a chemical bond, C(═O), C(═O)NH, (C1-C6) alkylene, or —(C1-C6) alkylene-O—;
L₃ is a chemical bond, 3- to 10-membered saturated or partially unsaturated heterocycloalkylene containing 1 to 4 heteroatoms independently selected from N, S, and O, 7- to 11-membered spiro heterocyclylene, or 5- to 11-membered bridged heterocyclylene, wherein the 3- to 10-membered saturated or partially unsaturated heterocycloalkylene, 7- to 11-membered spiro heterocyclylene, or 5- to 11-membered bridged heterocyclylene is optionally substituted with 1, 2, or 3 R_{L};
L₄ is a chemical bond, C(═O), C(═O)NH, (C1-C6) alkylene, or —(C1-C6) alkylene-O—;
L₅ is phenylene, 5- to 9-membered heteroarylene, 3- to 10-membered saturated or partially unsaturated heterocycloalkylene containing 1 to 4 heteroatoms independently selected from N, S, and O, 7- to 11-membered spiro heterocyclylene, or 5- to 11-membered bridged heterocyclylene, wherein the phenylene, 5- to 9-membered heteroarylene, 3- to 10-membered saturated or partially unsaturated heterocycloalkylene, 7- to 11-membered spiro heterocyclylene, or 5- to 11-membered bridged heterocyclylene is optionally substituted with 1, 2, or 3 R_{L}.

9. The compound or the isomer thereof, the crystalline form thereof, the pharmaceutically acceptable salt thereof, the hydrate thereof, or the solvate thereof according to claim 6, wherein in the general formula (1) or general formula (2),
L₁ is 3- to 10-membered saturated or partially unsaturated heterocycloalkylene containing 1 to 4 heteroatoms independently selected from N, S, and O, 7- to 11-membered spiro heterocyclylene, or 5- to 11-membered bridged heterocyclylene, wherein the 3- to 10-membered saturated or partially unsaturated heterocycloalkylene, 7- to 11-membered spiro heterocyclylene, or 5- to 11-membered bridged heterocyclylene is optionally substituted with 1, 2, or 3 R_{L};
L₂ is a chemical bond;
L₃ is 3- to 10-membered saturated or partially unsaturated heterocycloalkylene containing 1 to 4 heteroatoms independently selected from N, S, and O, 7- to 11-membered spiro heterocyclylene, or 5- to 11-membered bridged heterocyclylene, wherein the 3- to 10-membered saturated or partially unsaturated heterocycloalkylene, 7- to 11-membered spiro heterocyclylene, or 5- to 11-membered bridged heterocyclylene is optionally substituted with 1, 2, or 3 R_{L};
L₄ is a chemical bond;
L₅ is phenylene, 5- to 9-membered heteroarylene, or 3- to 10-membered saturated or partially unsaturated heterocycloalkylene containing 1 to 4 heteroatoms independently selected from N, S, and O, wherein the phenylene, 5- to 9-membered heteroarylene, or 3- to 10-membered saturated or partially unsaturated heterocycloalkylene is optionally substituted with 1, 2, or 3 R_{L}.

10. The compound or the isomer thereof, the crystalline form thereof, the pharmaceutically acceptable salt thereof, the hydrate thereof, or the solvate thereof according to claim 6 or 7, wherein in the general formula (1) or general formula (2), the structural unit 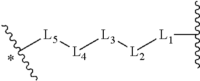
is selected from 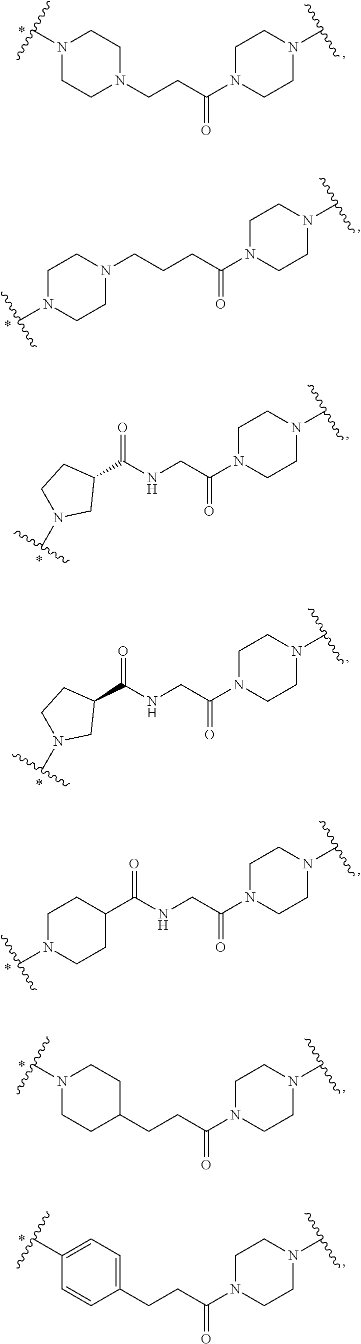 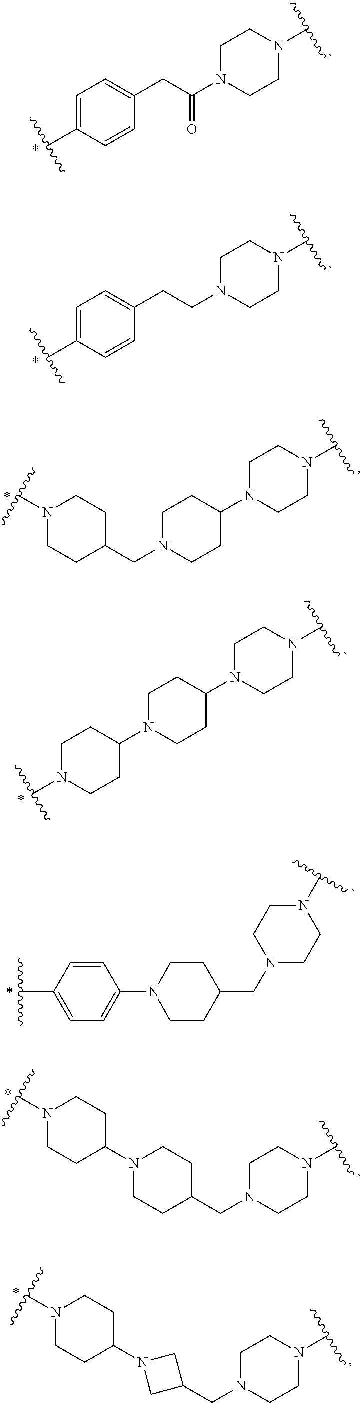 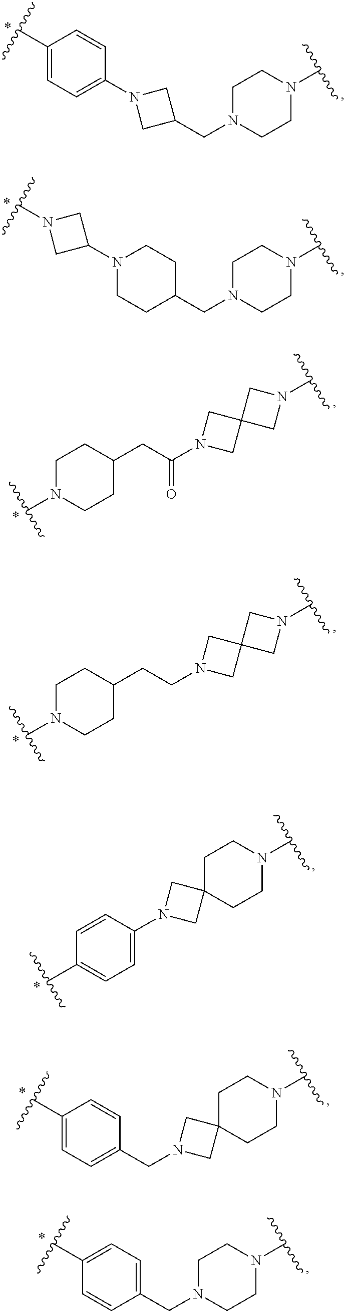 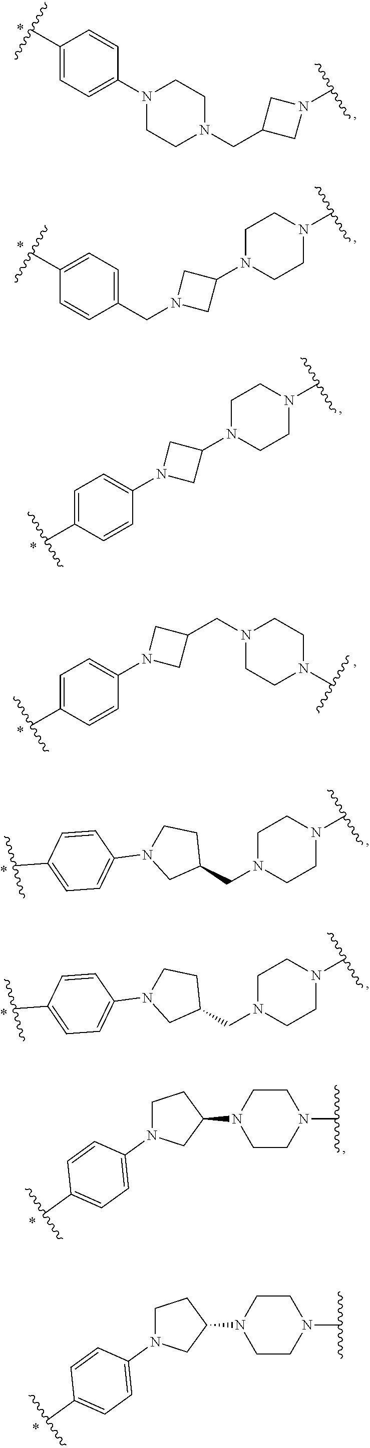 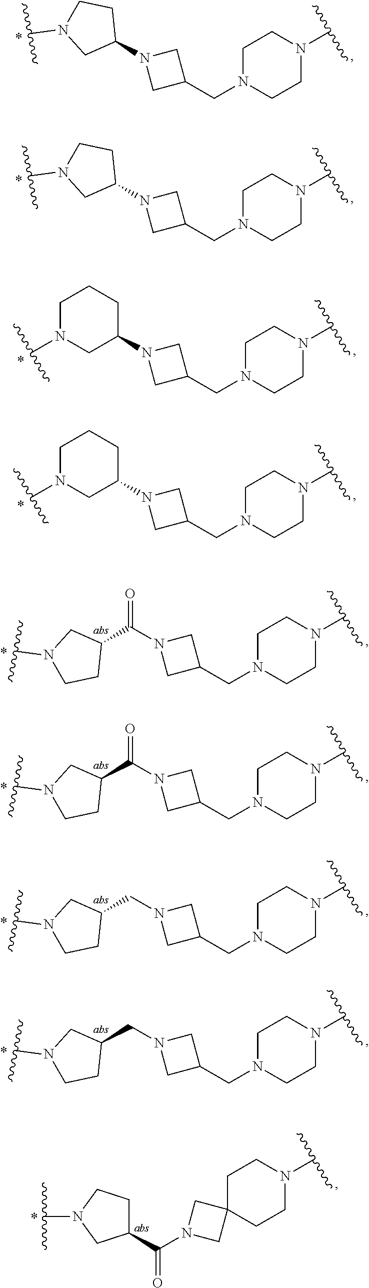 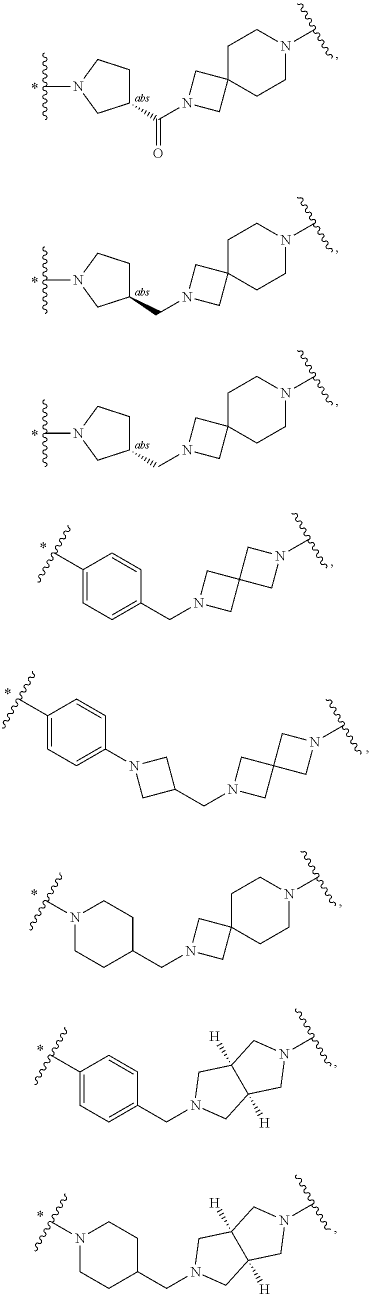 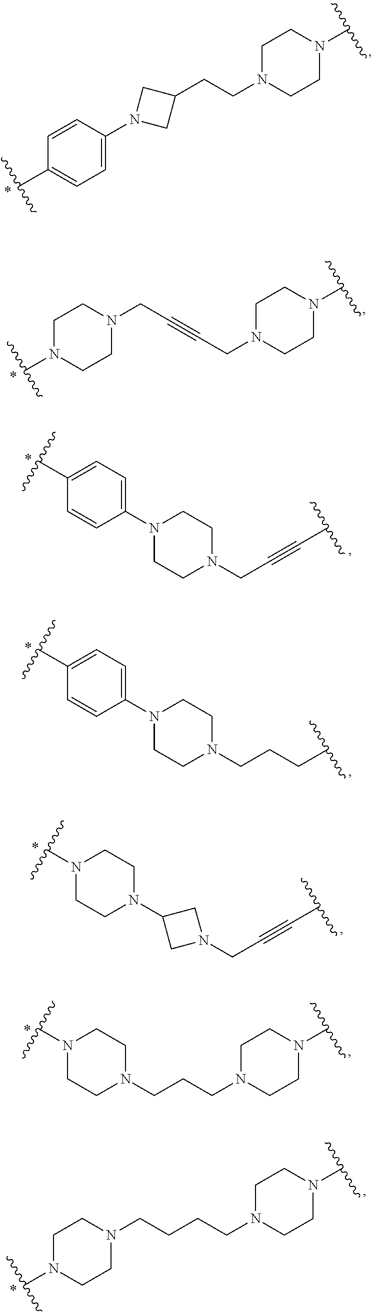 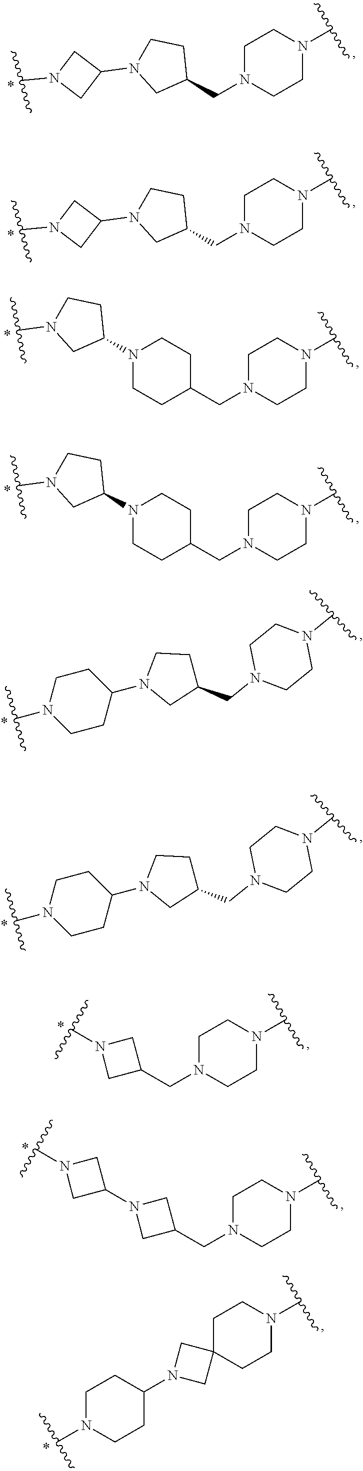 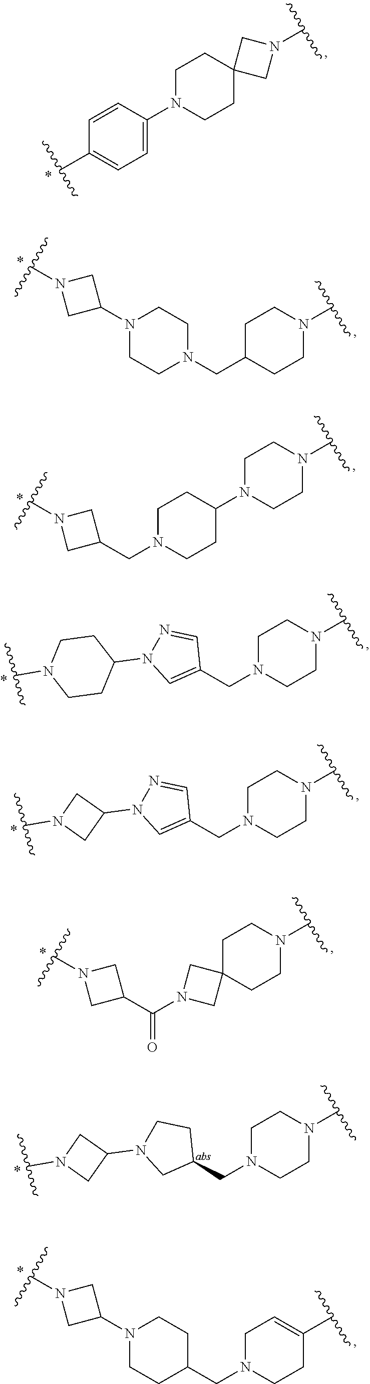 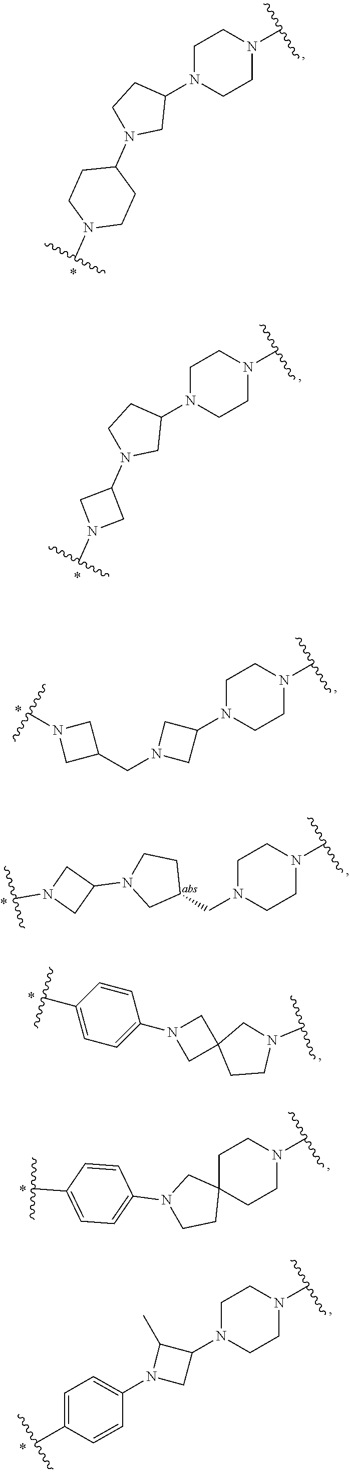 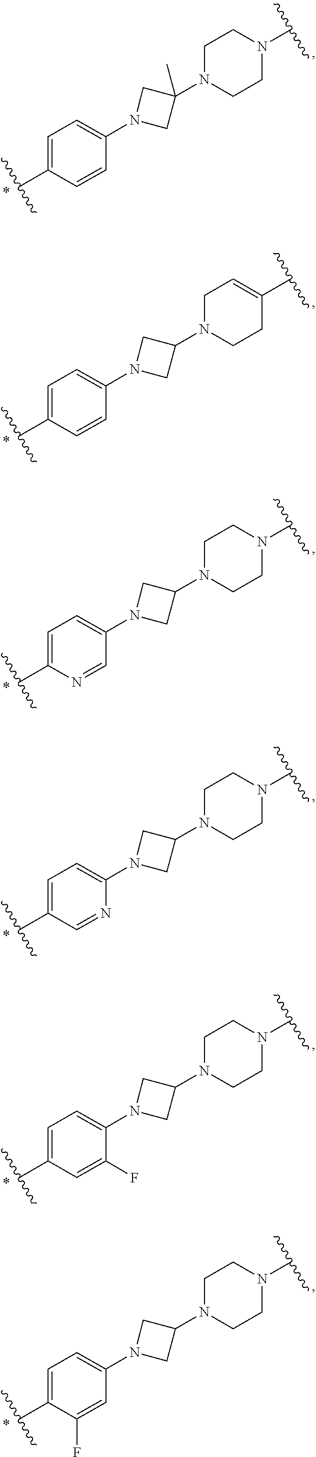 [SMILES: *c1ccc(N2CC(C)(N3CCN(*)CC3)C2)cc1 ; *C1=CCN(C2CN(c3ccc(*)cc3)C2)CC1 ; *c1ccc(N2CC(N3CCN(*)CC3)C2)cn1 ; *c1ccc(N2CC(N3CCN(*)CC3)C2)nc1 ; *c1ccc(N2CC(N3CCN(*)CC3)C2)c(F)c1 ; *c1ccc(N2CC(N3CCN(*)CC3)C2)cc1F] 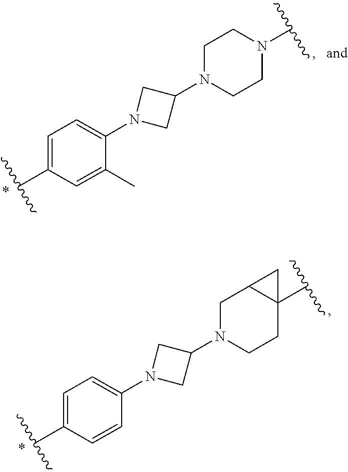 [SMILES: *c1ccc(N2CC(N3CCN(*)CC3)C2)c(C)c1 ; *c1ccc(N2CC(N3CCC4(*)CC4C3)C2)cc1]
wherein * denotes the connection to CLM.

11. The compound or the isomer thereof, the crystalline form thereof, the pharmaceutically acceptable salt thereof, the hydrate thereof, or the solvate thereof according to claim 11, wherein in the general formula (1) or general formula (2), each of R₁ and R₂ is independently selected from H, deuterium, alkyl, deuterated alkyl, haloalkyl, alkoxy, hydroxy, haloalkoxy, halogen, amino, carbamoyl, —(CH₂)ₙ₁Rₐ, —(CH₂)ₙ₁ORₐ, and —(CH₂)ₙ₁NRₐR_{b}, wherein the alkyl, deuterated alkyl, haloalkyl, alkoxy, haloalkoxy, amino, and carbamoyl are optionally further substituted with one or more substituents selected from deuterium, substituted or unsubstituted cycloalkylalkyl, substituted or unsubstituted cycloalkylhaloalkyl, halogen, substituted or unsubstituted cycloalkylamino, sulfydryl, oxo, cyano, hydroxy, substituted or unsubstituted cycloalkyl, substituted or unsubstituted heterocyclyl, —(CH₂)ₙ₁R_{c}, —(CH₂)ₙ₁OR_{c}, and —(CH₂)ₙ₁NR_{c}R_{d};
or any two adjacent or non-adjacent R₂ are linked to form one cycloalkyl or heterocycloalkyl group, wherein the cycloalkyl and heterocycloalkyl groups are optionally further substituted with one or more substituents selected from deuterium, substituted or unsubstituted alkyl, substituted or unsubstituted haloalkyl, halogen, substituted or unsubstituted amino, oxo, and hydroxy;
Rₐ, R_{b}, R_{c}, and R_{d} are each independently selected from hydrogen, deuterium, alkyl, deuterated alkyl, haloalkyl, alkoxy, hydroxyalkyl, haloalkoxy, halogen, hydroxy, amino, carbamoyl, cycloalkyl, and heterocycloalkyl, wherein the alkyl, deuterated alkyl, haloalkyl, alkoxy, hydroxyalkyl, haloalkoxy, cycloalkyl, and heterocycloalkyl are optionally further substituted with one or more substituents selected from deuterium, substituted or unsubstituted alkyl, halogen, hydroxy, substituted or unsubstituted amino, oxo, substituted or unsubstituted alkoxy, substituted or unsubstituted hydroxyalkyl, substituted or unsubstituted cycloalkyl, and substituted or unsubstituted heterocycloalkyl;
R₃ is H, halogen, cyclopropyl, or CH₃;
R₄ is 4- to 6-membered saturated heterocycloalkyl containing 1 to 2 heteroatoms independently selected from N, S, and O, phenyl, or 5- to 6-membered heteroaryl containing 1 to 4 heteroatoms independently selected from N, S, and O, wherein the 4- to 6-membered saturated heterocycloalkyl, phenyl, or 5- to 6-membered heteroaryl is optionally further substituted with one or more substituents selected from deuterium, alkyl, deuterated alkyl, haloalkyl, alkoxy, haloalkoxy, halogen, amino, nitro, hydroxy, cyano, cycloalkyl, heterocyclyl, oxoheterocycloalkyl, thioheterocycloalkyl, oxo, and thio; and
n is 0, 1, or 2;
m is 0, 1, or 2;
n1 is 0 or 1.

12. The compound or the isomer thereof, the crystalline form thereof, the pharmaceutically acceptable salt thereof, the hydrate thereof, or the solvate thereof according to claim 11, wherein in the general formula (1) or general formula (2), each of R₁ and R₂ is independently selected from H, deuterium, methyl, deuterated methyl, haloalkyl, methoxy, hydroxy, halomethoxy, F, Cl, amino, carbamoyl, 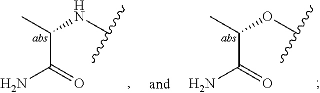
or when m is 2, two R₂ attached to the same carbon atom may form cyclopropyl.

13. The compound or the isomer thereof, the crystalline form thereof, the pharmaceutically acceptable salt thereof, the hydrate thereof, or the solvate thereof according to claim 11, wherein in the general formula (1) or general formula (2), R₃ is H, F, Cl, cyclopropyl, or CH₃.

14. The compound or the isomer thereof, the crystalline form thereof, the pharmaceutically acceptable salt thereof, the hydrate thereof, or the solvate thereof according to claim 6, wherein the general formula (1) or general formula (2) has a structure as shown in general formula (3): 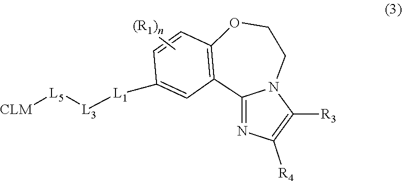
wherein
L₁ is 3- to 10-membered saturated or partially unsaturated heterocycloalkylene containing 1 to 4 heteroatoms independently selected from N, S, and O, 7- to 11-membered spiro heterocyclylene, or 5- to 11-membered bridged heterocyclylene, wherein the 3- to 10-membered saturated or partially unsaturated heterocycloalkylene, 7- to 11-membered spiro heterocyclylene, or 5- to 11-membered bridged heterocyclylene is optionally substituted with 1, 2, or 3 R_{L};
L₃ is 3- to 10-membered saturated or partially unsaturated heterocycloalkylene containing 1 to 4 heteroatoms independently selected from N, S, and O, 7- to 11-membered spiro heterocyclylene, or 5- to 11-membered bridged heterocyclylene, wherein the 3- to 10-membered saturated or partially unsaturated heterocycloalkylene, 7- to 11-membered spiro heterocyclylene, or 5- to 11-membered bridged heterocyclylene is optionally substituted with 1, 2, or 3 R_{L};
L₅ is phenylene, 5- to 9-membered heteroarylene, or 3- to 10-membered saturated or partially unsaturated heterocycloalkylene containing 1 to 4 heteroatoms independently selected from N, S, and O, wherein the phenylene, 5- to 9-membered heteroarylene, or 3- to 10-membered saturated or partially unsaturated heterocycloalkylene is optionally substituted with 1, 2, or 3 R_{L};
each R_{L} is independently selected from H, F, C1, hydroxy, amino, cyano, amido, methyl, methoxy, trifluoromethyl, difluoromethyl, monofluoromethyl, trifluoromethoxy, methylamino, and dimethylamino;
CLM is 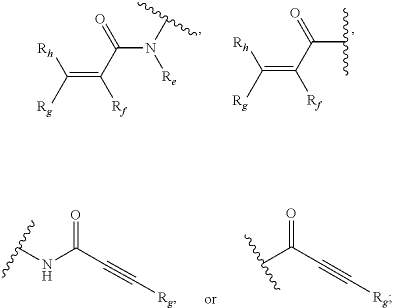
R₁ is independently selected from hydrogen, deuterium, (C1-C3) alkyl, (C1-C3) deuterated alkyl, (C1-C3) haloalkyl, (C1-C3) alkoxy, (C1-C3) haloalkoxy, halogen, amino, methylamino, hydroxy, cyano, (C3-C6) cycloalkyl, and (3- to 6-membered) heterocycloalkyl; n is 0, 1, or 2;
R₃ is H, halogen, (C1-C3) alkyl, or cyclopropyl;
R₄ is 4- to 6-membered heterocycloalkyl containing 1 to 2 heteroatoms independently selected from N, S, and O, phenyl, or 5- to 6-membered heteroaryl containing 1 to 4 heteroatoms independently selected from N, S, and O, wherein the 4- to 6-membered heterocycloalkyl, phenyl, or 5- to 6-membered heteroaryl is optionally further substituted with one or more substituents selected from deuterium, (C1-C3) alkyl, (C1-C3) deuterated alkyl, (C1-C3) haloalkyl, (C1-C3) alkoxy, (C1-C3) haloalkoxy, halogen, amino, nitro, hydroxy, cyano, (C3-C6) cycloalkyl, (3- to 6-membered) heterocycloalkyl, (3- to 6-membered) oxoheterocycloalkyl, (3- to 6-membered) thioheterocycloalkyl, oxo, and thio.

15. The compound or the isomer thereof, the crystalline form thereof, the pharmaceutically acceptable salt thereof, the hydrate thereof, or the solvate thereof according to claim 14, wherein in the general formula (3),
L₁ is 4- to 10-membered saturated or partially unsaturated heterocycloalkylene containing 1 to 4 heteroatoms independently selected from N, S, and O or 5- to 11-membered bridged heterocyclylene, wherein the 4- to 10-membered saturated or partially unsaturated heterocycloalkylene or 5- to 11-membered bridged heterocyclylene is optionally substituted with 1, 2, or 3 R_{L}; L₁ is preferably 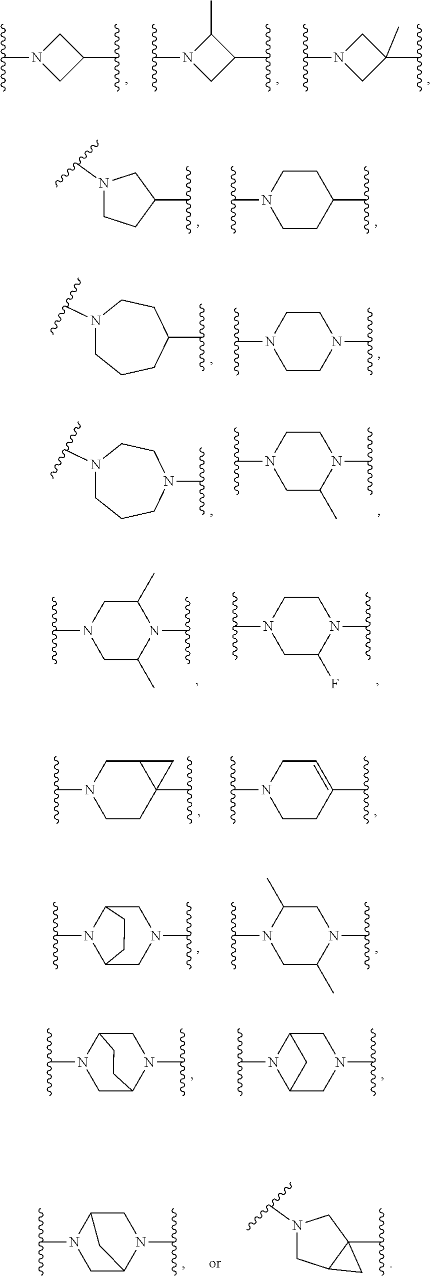
more preferably 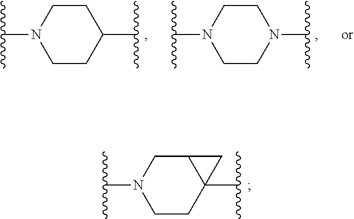
L₃ is 4- to 10-membered saturated or partially unsaturated heterocycloalkylene containing 1 to 4 heteroatoms independently selected from N, S, and O or 5- to 11-membered bridged heterocyclylene, wherein the 4- to 10-membered saturated or partially unsaturated heterocycloalkylene or 5- to 11-membered bridged heterocyclylene is optionally substituted with 1, 2, or 3 R_{L}; L₃ is preferrably 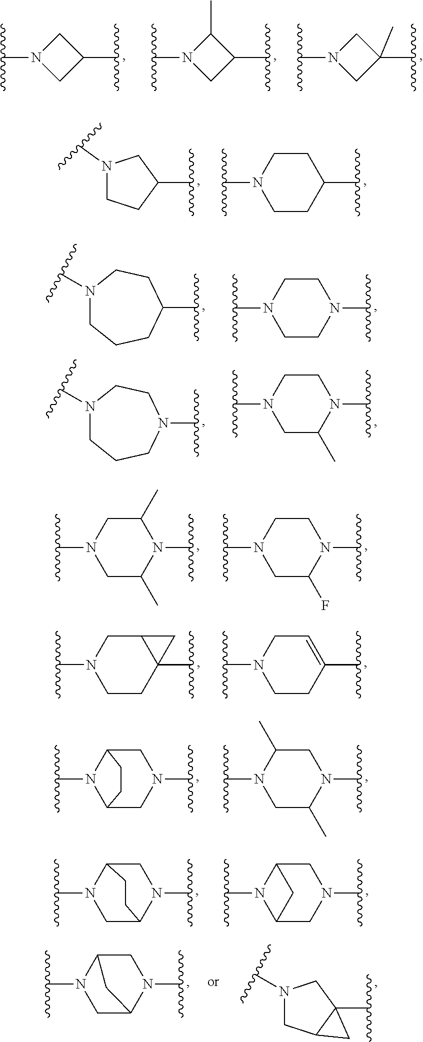
more preferably 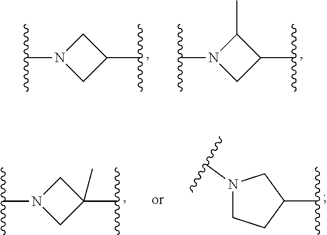
L₅ is phenylene, 5- to 6-membered heteroarylene, or 4- to 10-membered saturated or partially unsaturated heterocycloalkylene containing 1 to 4 heteroatoms independently selected from N, S, and O, wherein the phenylene, 5- to 6-membered heteroarylene, or 4- to 10-membered saturated or partially unsaturated heterocycloalkylene is optionally substituted with 1, 2, or 3 R_{L}; L₅ is preferably 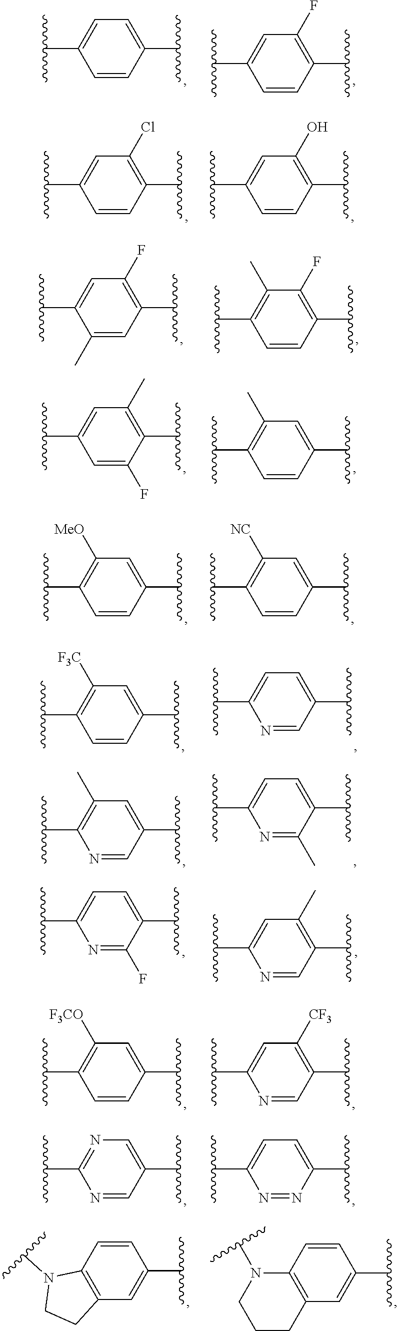 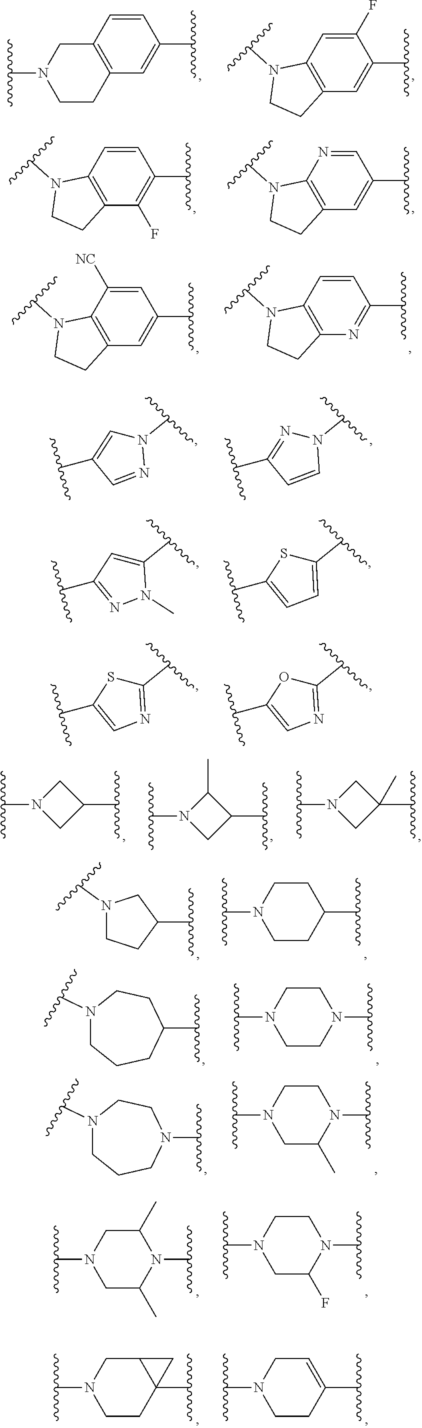 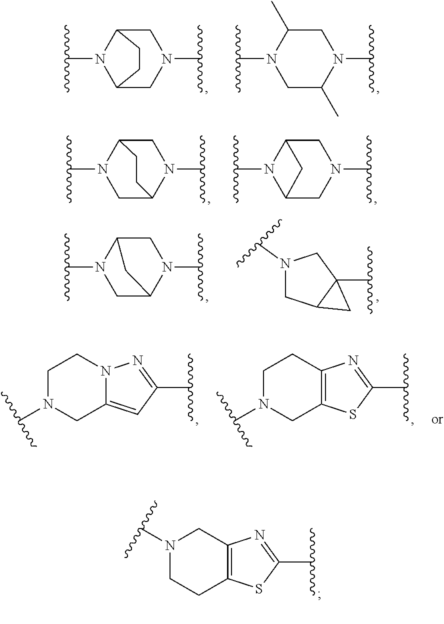
CLM is 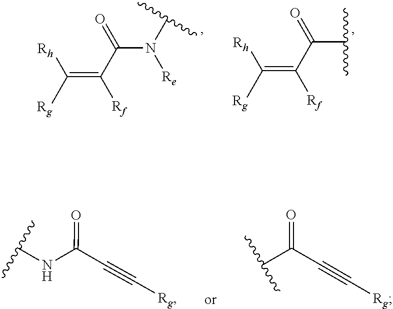
Rₑ is H or Me;
R_{f} is H, F, Me, or CN;
R_{g} and Rₕ are each independently H, halogen, —CN, —(CH₂)_{w}R, —(CH₂)_{w}OR, —(CH₂)_{w}N(R)₂, substituted or unsubstituted (C1-C6) alkyl, substituted or unsubstituted (C3-C6) cycloalkyl, or substituted or unsubstituted 3- to 7-membered saturated or partially unsaturated heterocycloalkyl containing 1 to 2 heteroatoms independently selected from N, S, and O, wherein the substituted or unsubstituted (C1-C6) alkyl is preferably 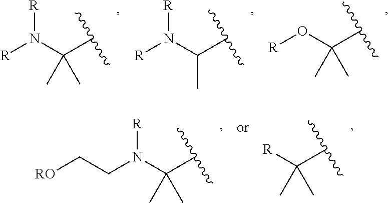
and the substituted or unsubstituted (C3-C6) cycloalkyl is preferably 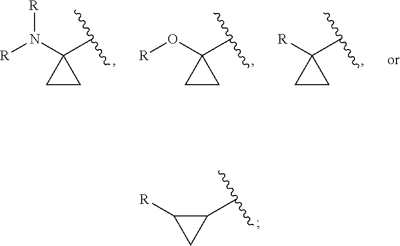
each R is independently H, (C1-C6) alkyl, (C3-C6) cycloalkyl, 3- to 7-membered saturated or partially unsaturated heterocycloalkyl containing 1 to 2 heteroatoms independently selected from N, S, and O, 7- to 11-membered spiro heterocycloalkyl, or 5- to 11-membered bridged heterocycloalkyl, wherein the (C1-C6) alkyl, (C3-C6) cycloalkyl, 3- to 7-membered saturated or partially unsaturated heterocycloalkyl containing 1 to 2 heteroatoms independently selected from N, S, and O, 7- to 11-membered spiro heterocycloalkyl, or 5- to 11-membered bridged heterocycloalkyl is optionally further substituted with one or more substituents selected from deuterium, (C1-C6) alkyl, (C1-C6) alkoxy, (C3-C6) cycloalkyl, 3- to 7-membered saturated or partially unsaturated heterocycloalkyl containing 1 to 2 heteroatoms independently selected from N, S, and O, 7- to 11-membered spiro heterocyclyl, 5- to 11-membered bridged heterocyclyl, oxo, —C(O)Me, —C(O)OMe, —C(O)OBu-t, —F, C1, —OH, and —NH₂;
w is 0, 1, or 2;
R₁ is independently selected from hydrogen, deuterium, -Me, —OMe, —OCD₃, —CD₃, —CHF₂, —CF₃, F, Cl, —NH₂, —NHMe, hydroxy, cyano, and cyclopropyl; n is 0, 1, or 2;
R₃ is H, F, Cl, -Me, or cyclopropyl;
R₄ is 5-membered heterocycloalkyl containing 1 to 2 heteroatoms independently selected from N, S, and O or 5-membered heteroaryl containing 1 to 3 heteroatoms independently selected from N, S, and O, wherein the 5-membered heterocycloalkyl or 5-membered heteroaryl is optionally further substituted with one or more substituents selected from deuterium, (C1-C3) alkyl, (C1-C3) deuterated alkyl, (C1-C3) haloalkyl, (C1-C3) alkoxy, (C1-C3) haloalkoxy, halogen, amino, nitro, hydroxy, cyano, (C3-C6) cycloalkyl, (3- to 6-membered) heterocycloalkyl, (3- to 6-membered) oxoheterocycloalkyl, (3- to 6-membered) thioheterocycloalkyl, oxo, and thio.

16. The compound or the isomer thereof, the crystalline form thereof, the pharmaceutically acceptable salt thereof, the hydrate thereof, or the solvate thereof according to claim 14, wherein in the general formula (3),
L₁ is 4- to 10-membered saturated or partially unsaturated heterocycloalkylene containing 1 to 4 heteroatoms independently selected from N, S, and O or 5- to 11-membered bridged heterocyclylene, wherein the 4- to 10-membered saturated or partially unsaturated heterocycloalkylene or 5- to 11-membered bridged heterocyclylene is optionally substituted with 1, 2, or 3 R_{L}; L₁ is preferably 4- to 7-membered saturated or partially unsaturated heterocycloalkylene containing 1 to 2 N atoms or 6- to 9-membered bridged heterocyclylene containing 1 to 2 N atoms, wherein the 4- to 7-membered saturated or partially unsaturated heterocycloalkylene or 6- to 9-membered bridged heterocyclylene is optionally substituted with 1 to 2 R_{L}; L₁ is preferably 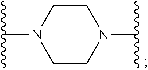 [SMILES: *N1CCN(*)CC1]
L₃ is 4- to 10-membered saturated or partially unsaturated heterocycloalkylene containing 1 to 4 heteroatoms independently selected from N, S, and O or 5- to 11-membered bridged heterocyclylene, wherein the 4- to 10-membered saturated or partially unsaturated heterocycloalkylene or 5- to 11-membered bridged heterocyclylene is optionally substituted with 1, 2, or 3 R_{L}; L₃ is preferably 4- to 7-membered saturated or partially unsaturated heterocycloalkylene containing 1 to 2 N atoms or 6- to 9-membered bridged heterocyclylene containing 1 to 2 N atoms, wherein the 4- to 7-membered saturated or partially unsaturated heterocycloalkylene or 6- to 9-membered bridged heterocyclylene is optionally substituted with 1 to 2 R_{L}; L₃ is preferably 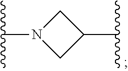 [SMILES: *C1CN(*)C1]
L₅ is phenylene, 5- to 6-membered heteroarylene containing 1 to 2 heteroatoms independently selected from N, S, and O, or 4- to 10-membered saturated or partially unsaturated heterocycloalkylene containing 1 to 4 heteroatoms independently selected from N, S, and O, wherein the phenylene, 5- to 6-membered heteroarylene, or 4- to 10-membered saturated or partially unsaturated heterocycloalkylene is optionally substituted with 1, 2, or 3 R_{L}; L₅ is preferably phenylene, 5- to 6-membered heteroarylene containing 1 to 2 heteroatoms independently selected from N, S, and O, or 8- to 10-membered partially unsaturated bicyclic heterocycloalkylene containing 1 to 3 heteroatoms independently selected from N, S, and O, wherein the phenylene, 5- to 6-membered heteroarylene, or 8- to 10-membered partially unsaturated bicyclic heterocycloalkylene is optionally substituted with 1 to 2 R_{L}; L₅ is preferably 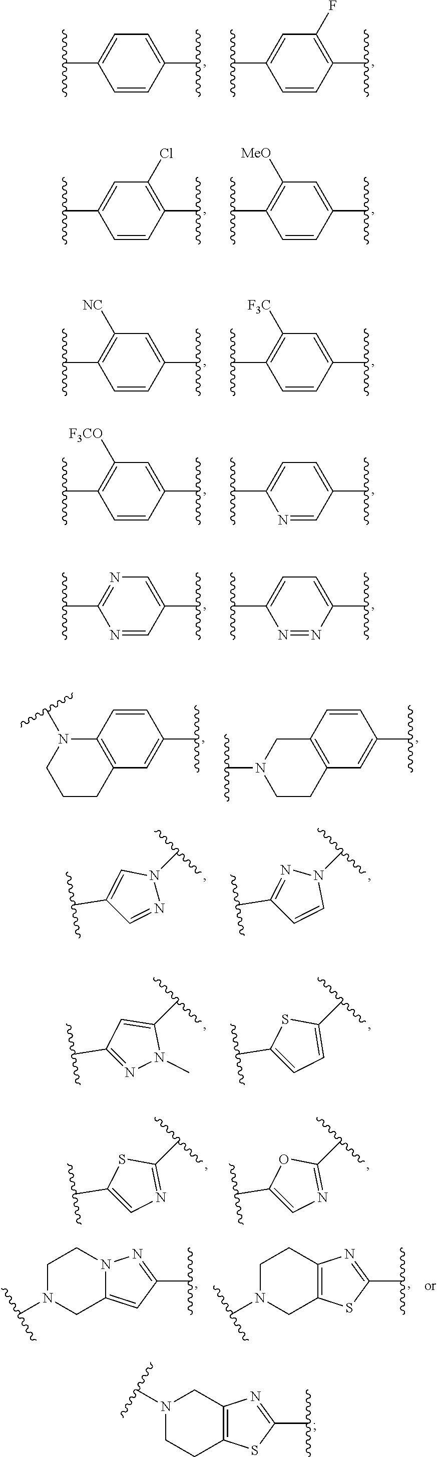
each R_{L} is independently selected from H, F, Cl, hydroxy, amino, cyano, amido, methyl, methoxy, trifluoromethyl, difluoromethyl, monofluoromethyl, trifluoromethoxy, methylamino, and dimethylamino;
CLM is 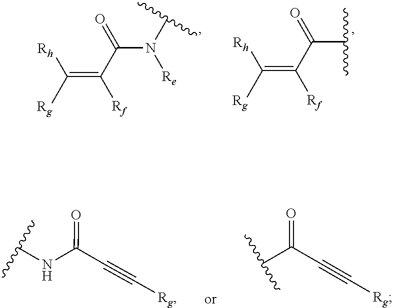
Rₑ is H or Me;
R_{f} is H, F, Me, or CN;
R_{g} and Rₕ are each independently H, halogen, —CN, —(CH₂)_{w}R, —(CH₂)_{w}OR, —(CH₂)_{w}N(R)₂, substituted or unsubstituted (C1-C6) alkyl, substituted or unsubstituted (C3-C6) cycloalkyl, or substituted or unsubstituted 3- to 7-membered saturated or partially unsaturated heterocycloalkyl containing 1 to 2 heteroatoms independently selected from N, S, and O, wherein the substituted or unsubstituted (C1-C6) alkyl is preferably 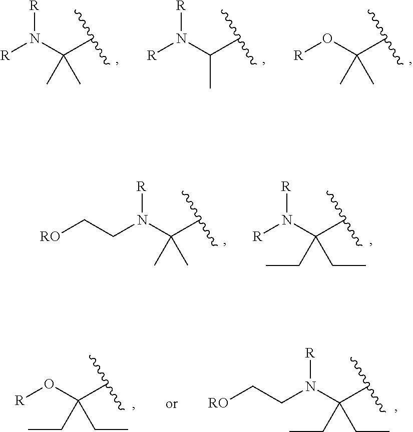
and the substituted or unsubstituted (C3-C6) cycloalkyl is preferably 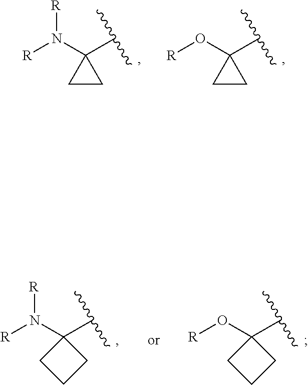
each R is independently H, —C(O)Rₘ, —C(O)ORₘ, —S(O)₂Rₘ, —CH₂OC(O)Rₙ, 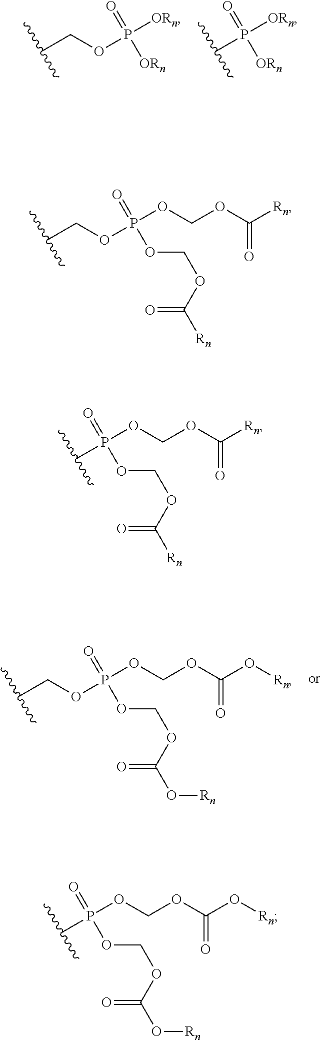
Rₘ and Rₙ are each independently H, (C1-C18) alkyl, (C3-C6) cycloalkyl, 3- to 7-membered saturated or partially unsaturated heterocycloalkyl containing 1 to 2 heteroatoms independently selected from N, S, and O, 7- to 11-membered spiro heterocycloalkyl, or 5- to 11-membered bridged heterocycloalkyl, wherein the (C1-C18) alkyl, (C3-C6) cycloalkyl, 3- to 7-membered saturated or partially unsaturated heterocycloalkyl containing 1 to 2 heteroatoms independently selected from N, S, and O, 7- to 11-membered spiro heterocycloalkyl, or 5- to 11-membered bridged heterocycloalkyl is optionally further substituted with one or more substituents selected from deuterium, (C1-C6) alkyl, (C1-C6) alkoxy, (C3-C6) cycloalkyl, 3- to 7-membered saturated or partially unsaturated heterocycloalkyl containing 1 to 2 heteroatoms independently selected from N, S, and O, 7- to 11-membered spiro heterocyclyl, 5- to 11-membered bridged heterocyclyl, oxo, —C(O)Me, —C(O)OMe, —C(O)OBu-t, —F, Cl, —OH, and —NH₂; Rₘ and Rₙ are each independently preferably H, methyl, ethyl, tert-butyl, isopropyl, cyclopropyl, or cyclobutyl;
w is 0, 1, or 2;
R₁ is independently selected from hydrogen, deuterium, -Me, —OMe, —OCD₃, —CD₃, —CHF₂, —CF₃, F, Cl, —NH₂, —NHMe, hydroxy, cyano, and cyclopropyl; n is 0, 1, or 2;
R₃ is H, F, Cl, -Me, or cyclopropyl;
R₄ is 5-membered heterocycloalkyl containing 1 to 2 heteroatoms independently selected from N, S, and O or 5-membered heteroaryl containing 1 to 3 heteroatoms independently selected from N, S, and O, wherein the 5-membered heterocycloalkyl or 5-membered heteroaryl is optionally further substituted with one or more substituents selected from deuterium, (C1-C3) alkyl, (C1-C3) deuterated alkyl, (C1-C3) haloalkyl, (C1-C3) alkoxy, (C1-C3) haloalkoxy, halogen, amino, nitro, hydroxy, cyano, (C3-C6) cycloalkyl, (3- to 6-membered) heterocycloalkyl, (3- to 6-membered) oxoheterocycloalkyl, (3- to 6-membered) thioheterocycloalkyl, oxo, and thio.

17. The compound or the isomer thereof, the crystalline form thereof, the pharmaceutically acceptable salt thereof, the hydrate thereof, or the solvate thereof according to claim 1, wherein in the general formulas (1)-(3), R₄ is selected from

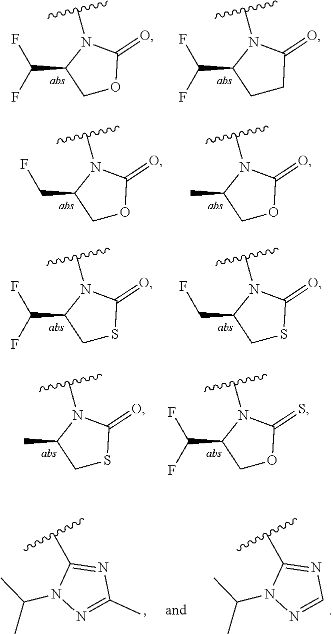

18. The compound or the isomer thereof, the crystalline form thereof, the pharmaceutically acceptable salt thereof, the hydrate thereof, or the solvate thereof according to claim 1, wherein the compound has one of the following structures:

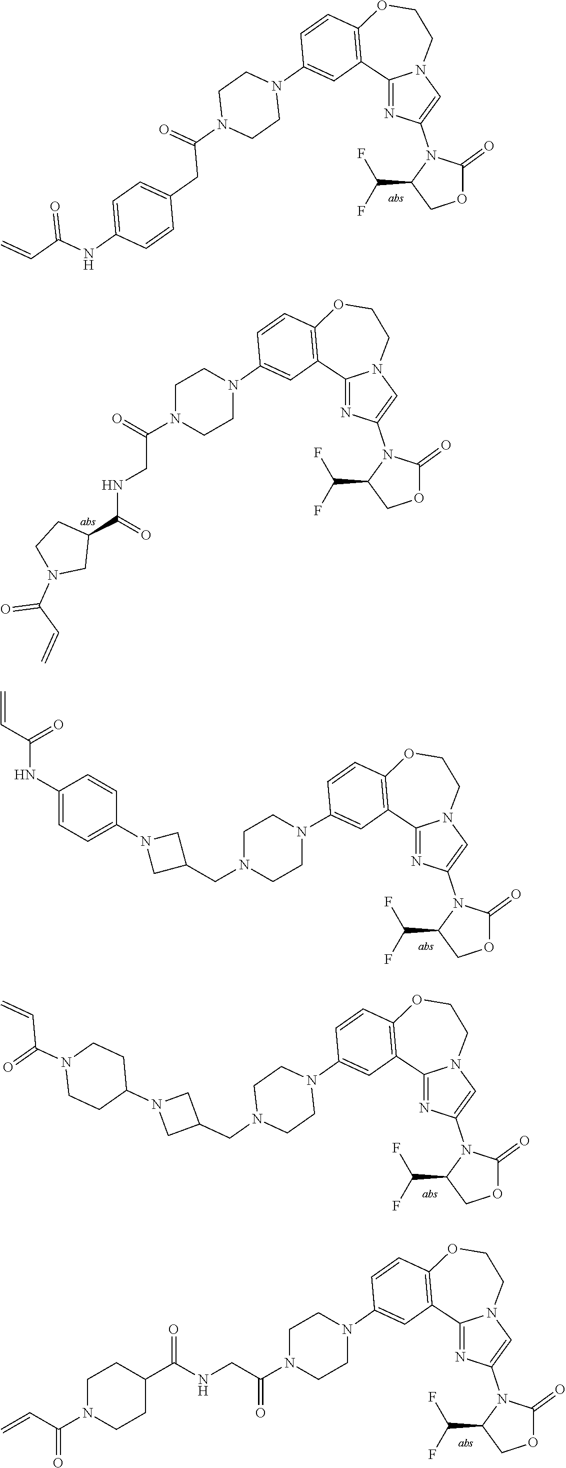 [SMILES: C=CC(=O)Nc1ccc(CC(=O)N2CCN(c3ccc4c(c3)-c3nc(N5C(=O)OC[C@H]5C(F)F)cn3CCO4)CC2)cc1 ; C=CC(=O)N1CC[C@@H](C(=O)NCC(=O)N2CCN(c3ccc4c(c3)-c3nc(N5C(=O)OC[C@H]5C(F)F)cn3CCO4)CC2)C1 ; C=CC(=O)Nc1ccc(N2CC(CN3CCN(c4ccc5c(c4)-c4nc(N6C(=O)OC[C@H]6C(F)F)cn4CCO5)CC3)C2)cc1 ; C=CC(=O)N1CCC(N2CC(CN3CCN(c4ccc5c(c4)-c4nc(N6C(=O)OC[C@H]6C(F)F)cn4CCO5)CC3)C2)CC1 ; C=CC(=O)N1CCC(C(=O)NCC(=O)N2CCN(c3ccc4c(c3)-c3nc(N5C(=O)OC[C@H]5C(F)F)cn3CCO4)CC2)CC1]

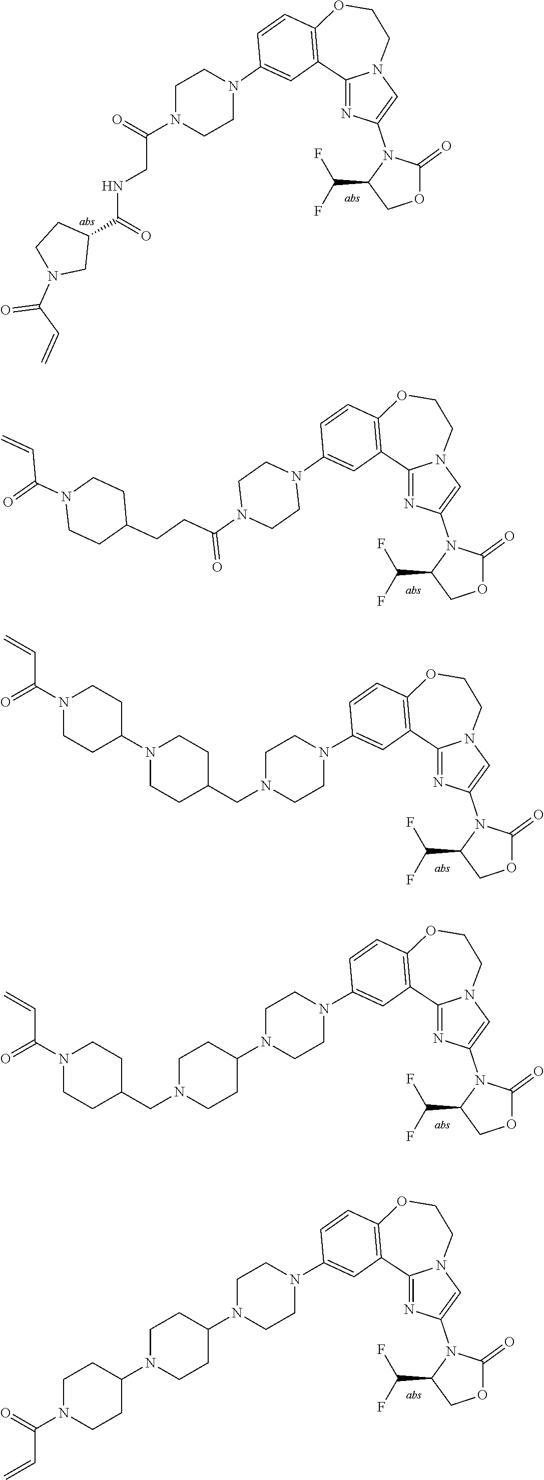

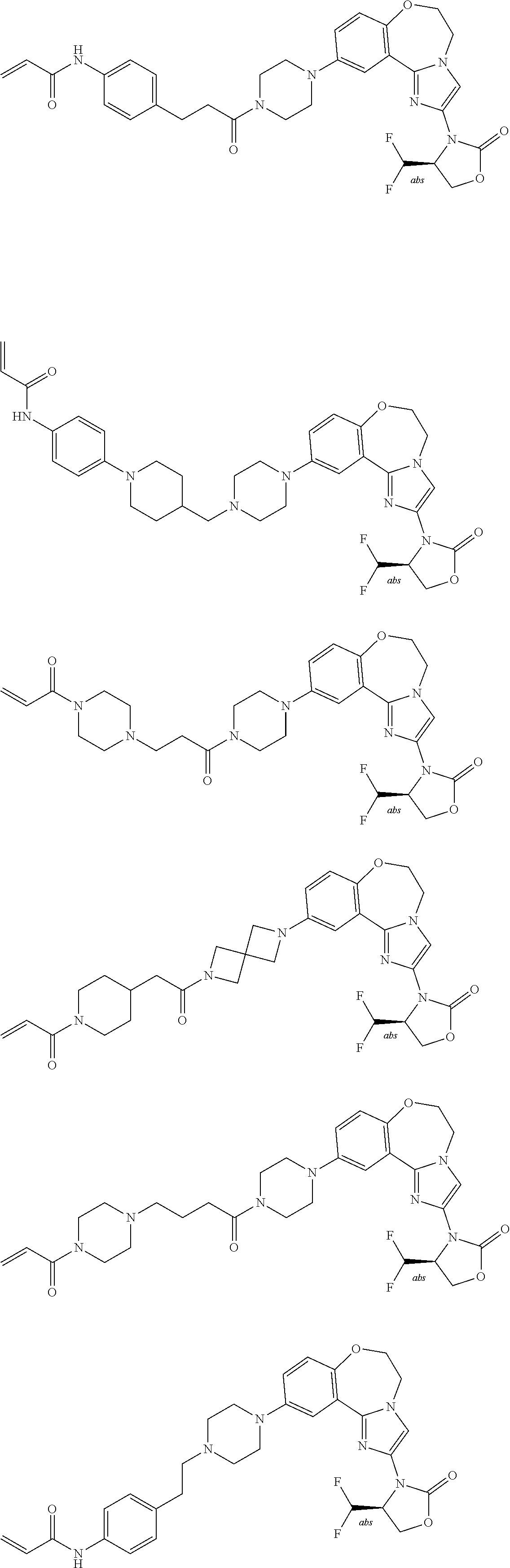

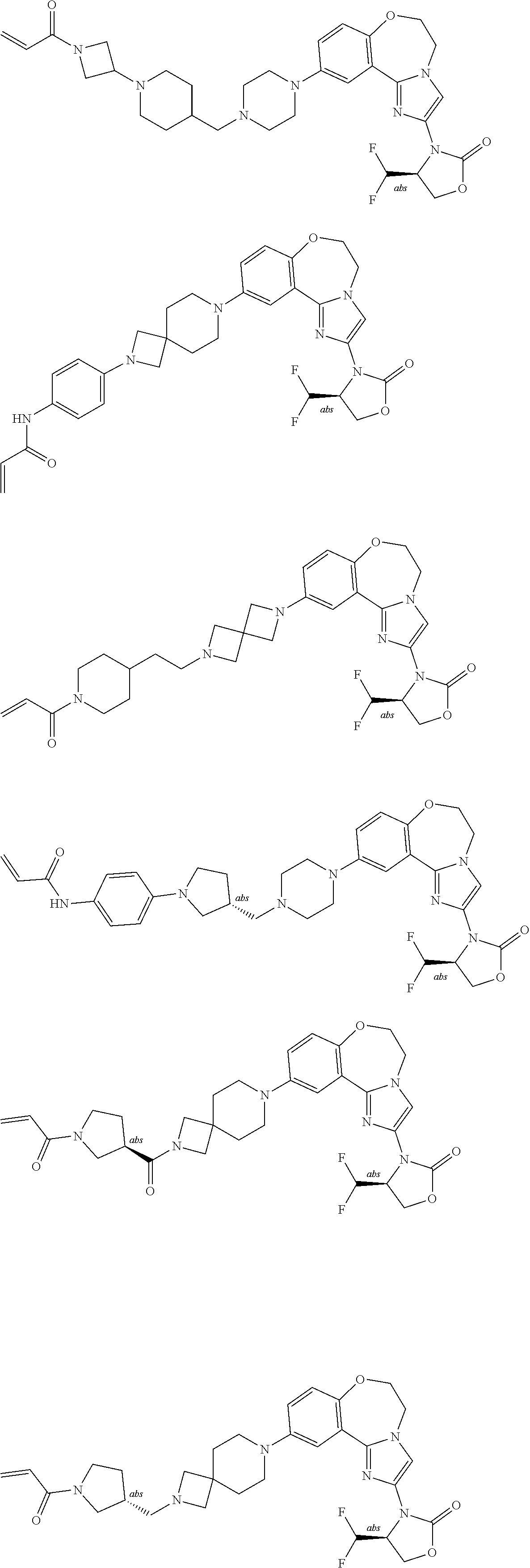

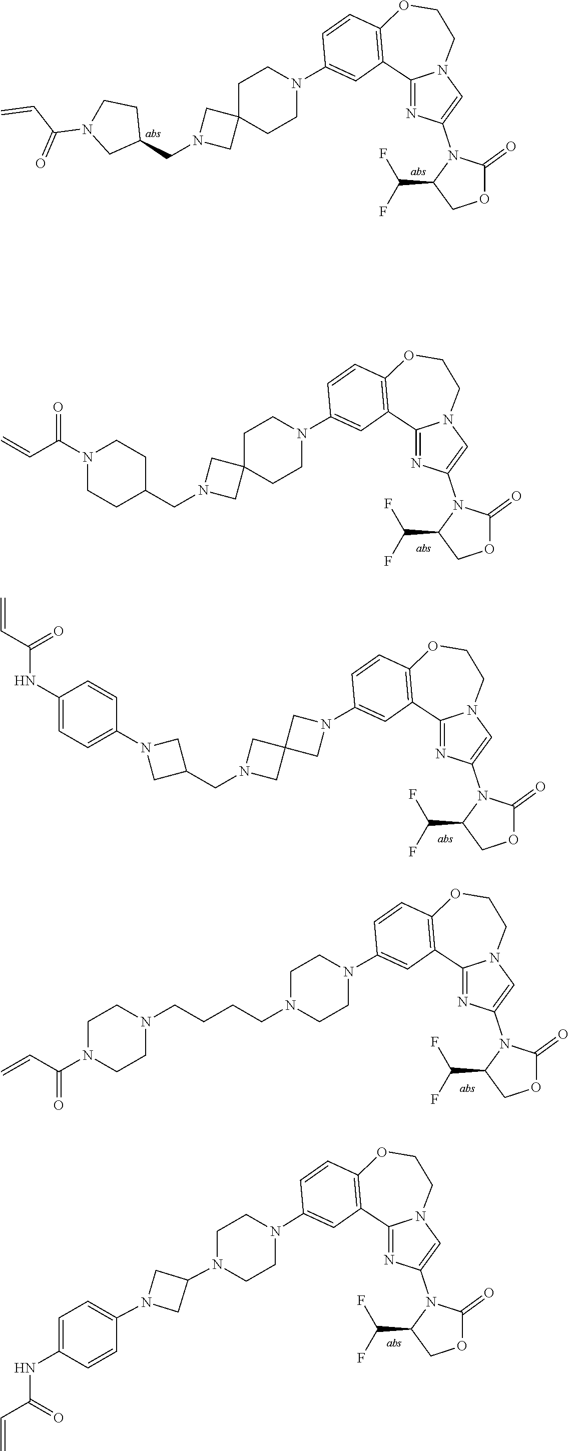

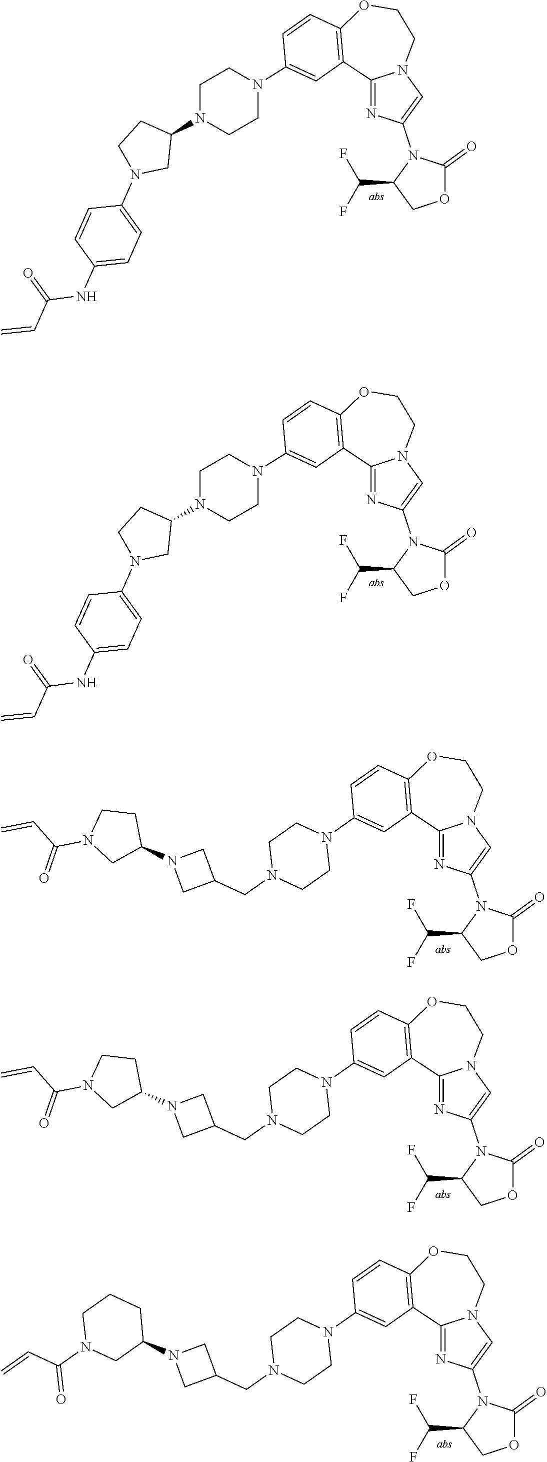

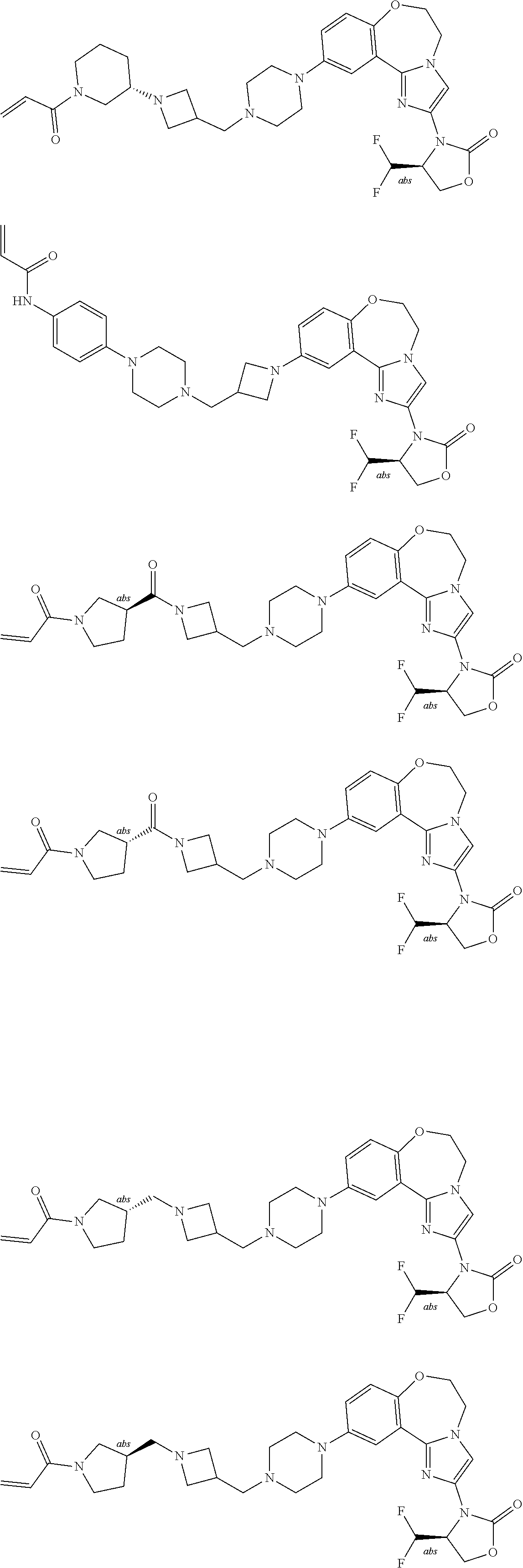

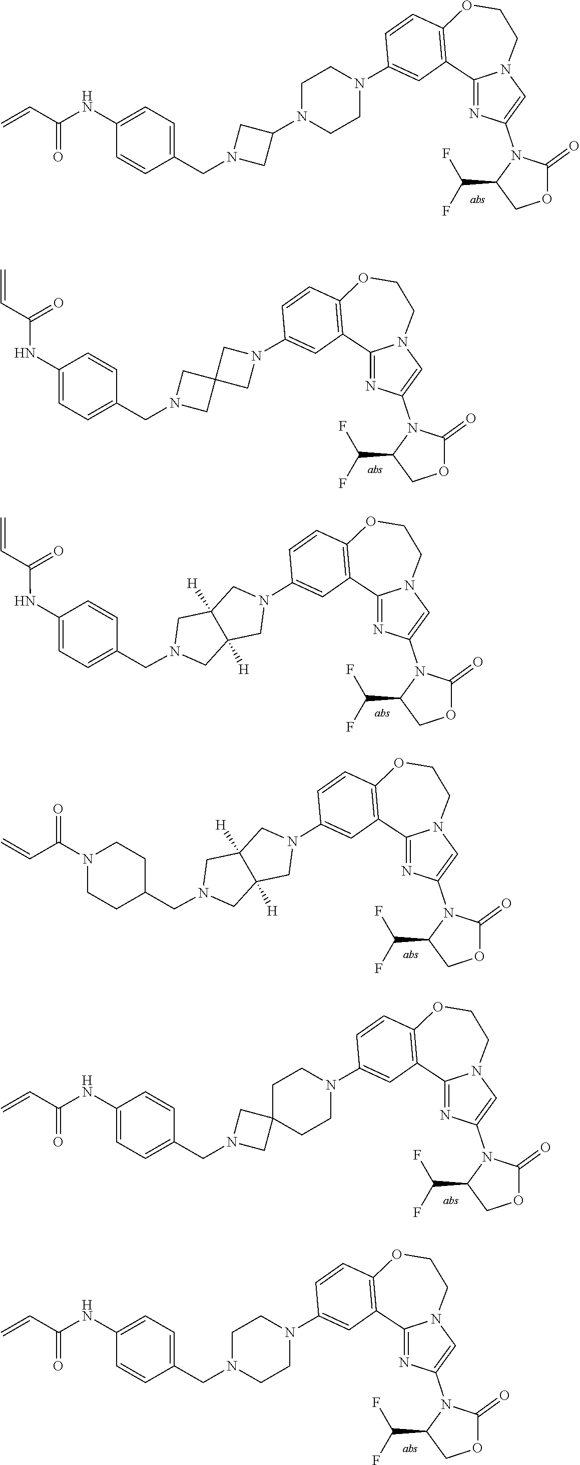

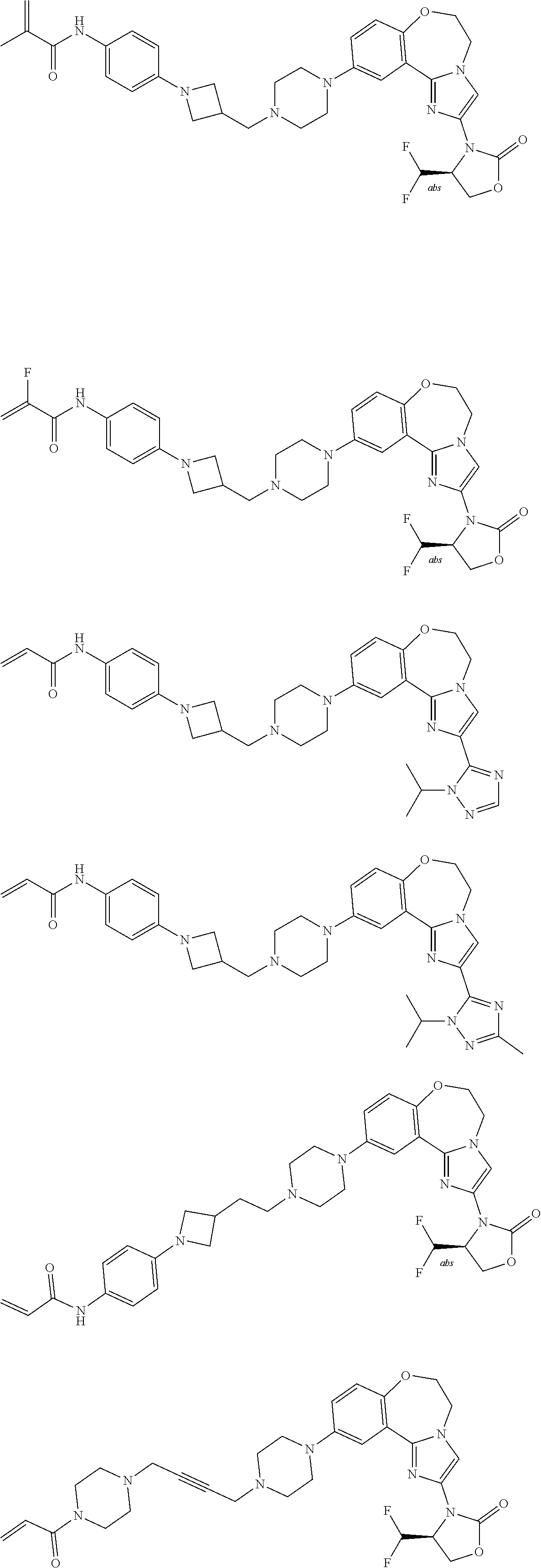

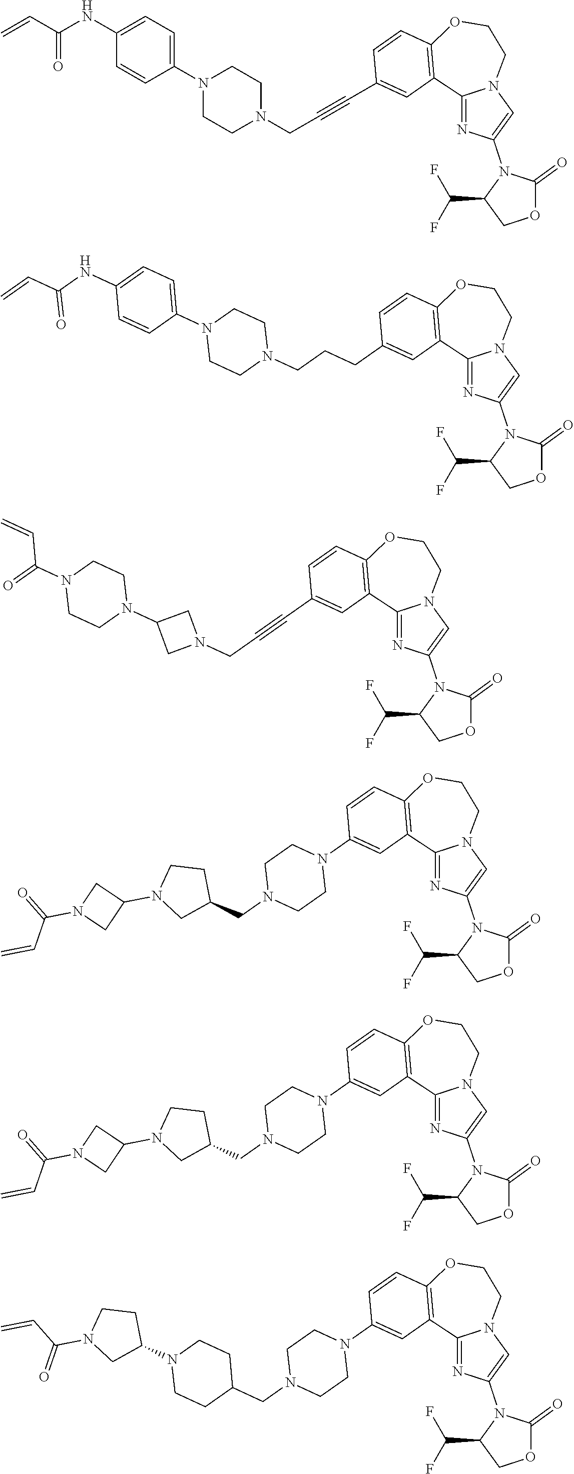

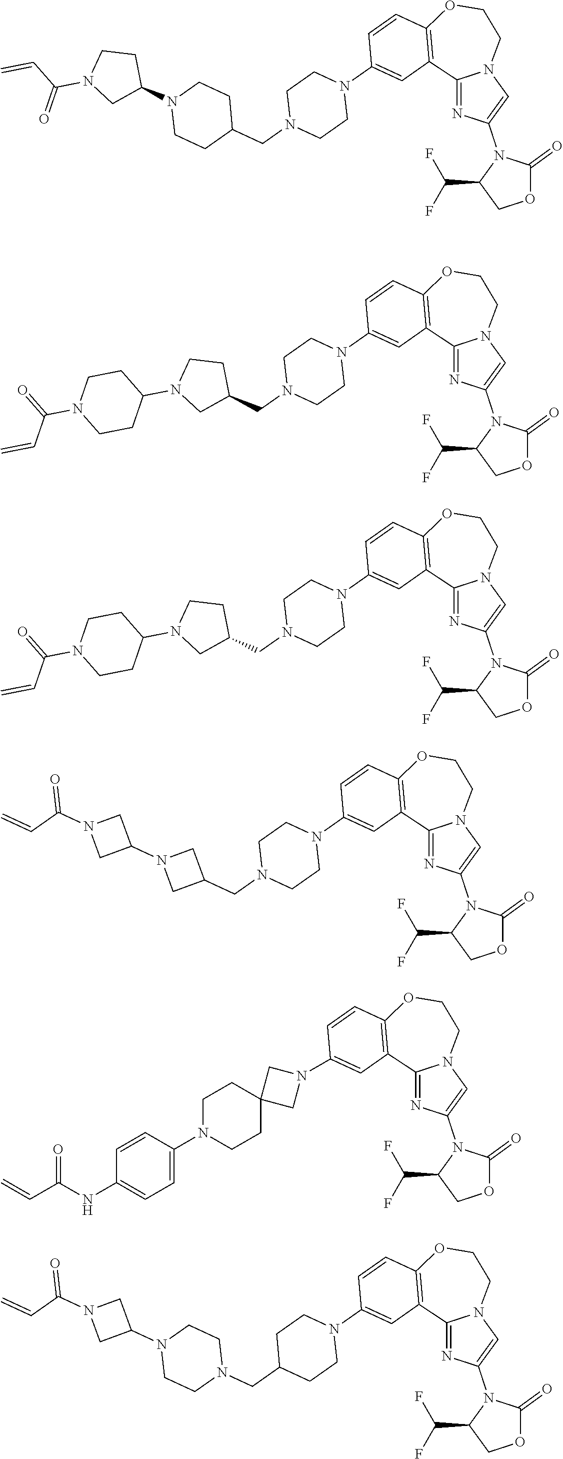

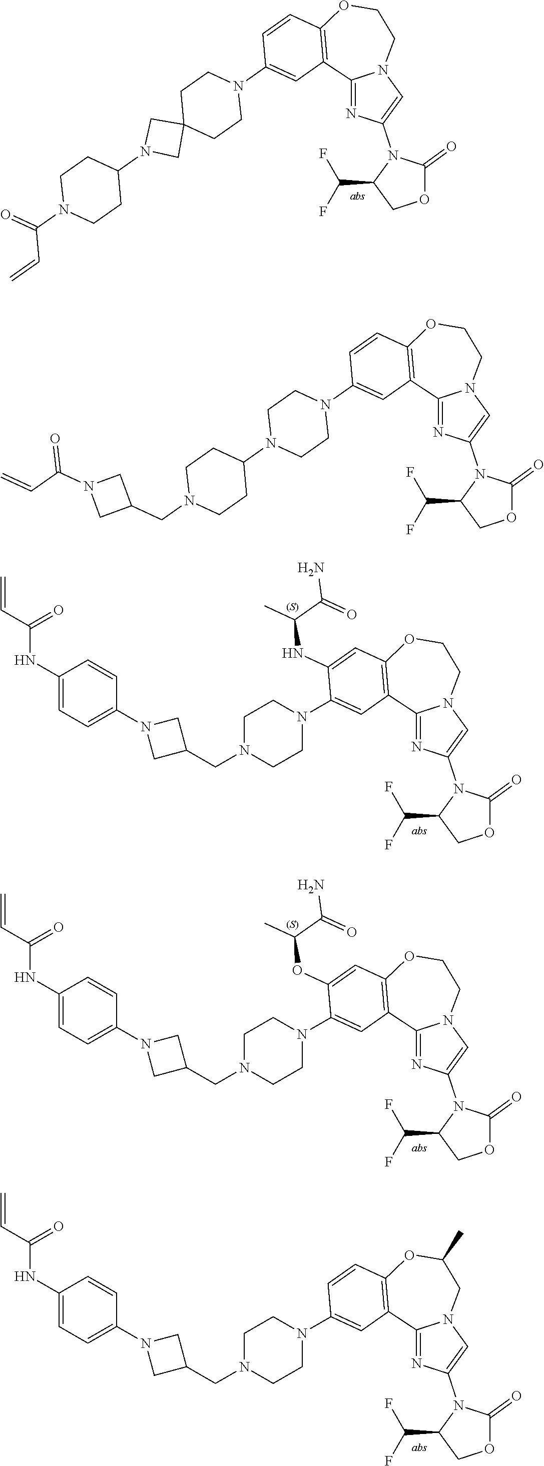

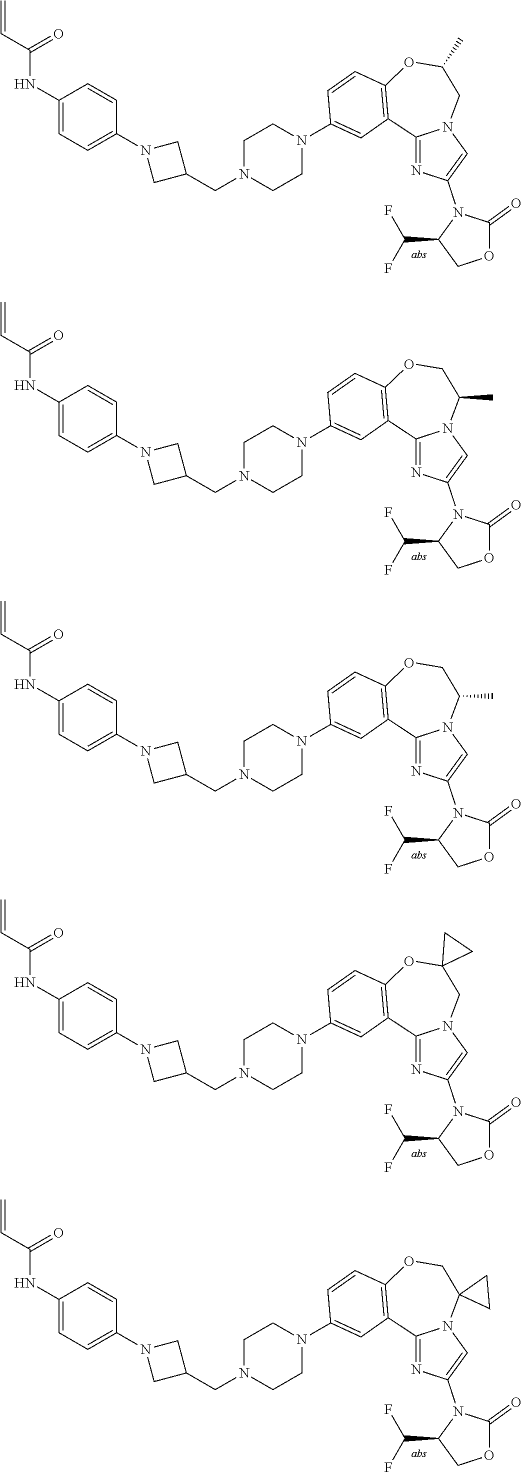 [SMILES: C=CC(=O)Nc1ccc(N2CC(CN3CCN(c4ccc5c(c4)-c4nc(N6C(=O)OC[C@H]6C(F)F)cn4C[C@@H](C)O5)CC3)C2)cc1 ; C=CC(=O)Nc1ccc(N2CC(CN3CCN(c4ccc5c(c4)-c4nc(N6C(=O)OC[C@H]6C(F)F)cn4[C@H](C)CO5)CC3)C2)cc1 ; C=CC(=O)Nc1ccc(N2CC(CN3CCN(c4ccc5c(c4)-c4nc(N6C(=O)OC[C@H]6C(F)F)cn4[C@@H](C)CO5)CC3)C2)cc1 ; C=CC(=O)Nc1ccc(N2CC(CN3CCN(c4ccc5c(c4)-c4nc(N6C(=O)OC[C@H]6C(F)F)cn4CC4(CC4)O5)CC3)C2)cc1 ; C=CC(=O)Nc1ccc(N2CC(CN3CCN(c4ccc5c(c4)-c4nc(N6C(=O)OC[C@H]6C(F)F)cn4C4(CC4)CO5)CC3)C2)cc1]

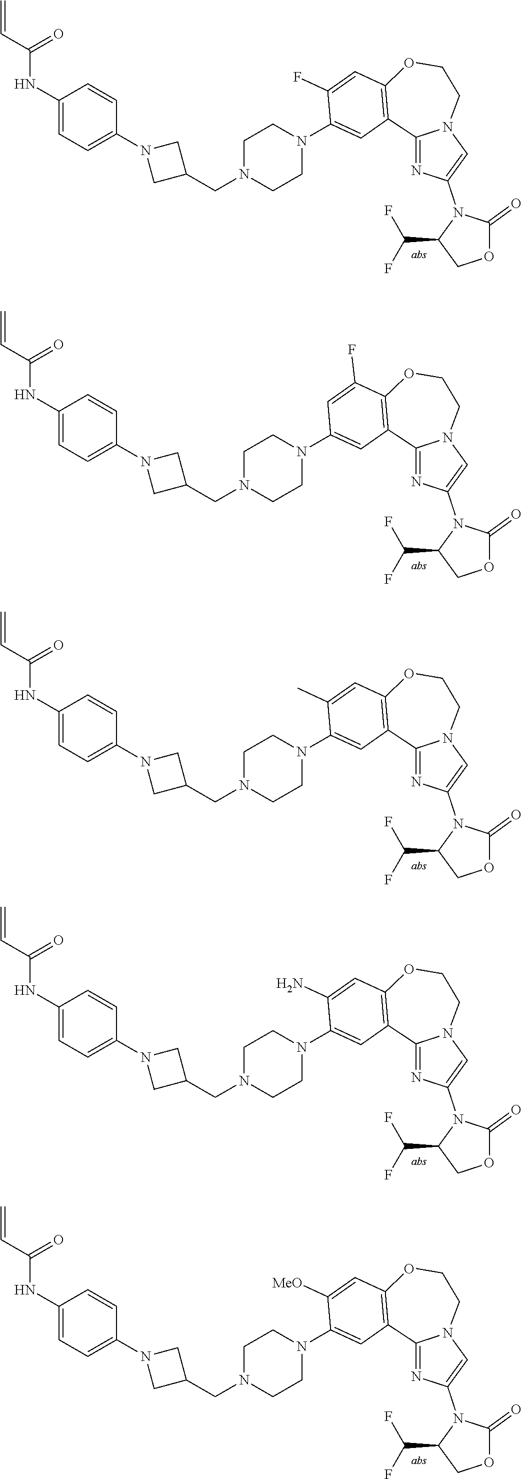 [SMILES: C=CC(=O)Nc1ccc(N2CC(CN3CCN(c4cc5c(cc4F)OCCn4cc(N6C(=O)OC[C@H]6C(F)F)nc4-5)CC3)C2)cc1 ; C=CC(=O)Nc1ccc(N2CC(CN3CCN(c4cc(F)c5c(c4)-c4nc(N6C(=O)OC[C@H]6C(F)F)cn4CCO5)CC3)C2)cc1 ; C=CC(=O)Nc1ccc(N2CC(CN3CCN(c4cc5c(cc4C)OCCn4cc(N6C(=O)OC[C@H]6C(F)F)nc4-5)CC3)C2)cc1 ; C=CC(=O)Nc1ccc(N2CC(CN3CCN(c4cc5c(cc4N)OCCn4cc(N6C(=O)OC[C@H]6C(F)F)nc4-5)CC3)C2)cc1 ; C=CC(=O)Nc1ccc(N2CC(CN3CCN(c4cc5c(cc4OC)OCCn4cc(N6C(=O)OC[C@H]6C(F)F)nc4-5)CC3)C2)cc1]

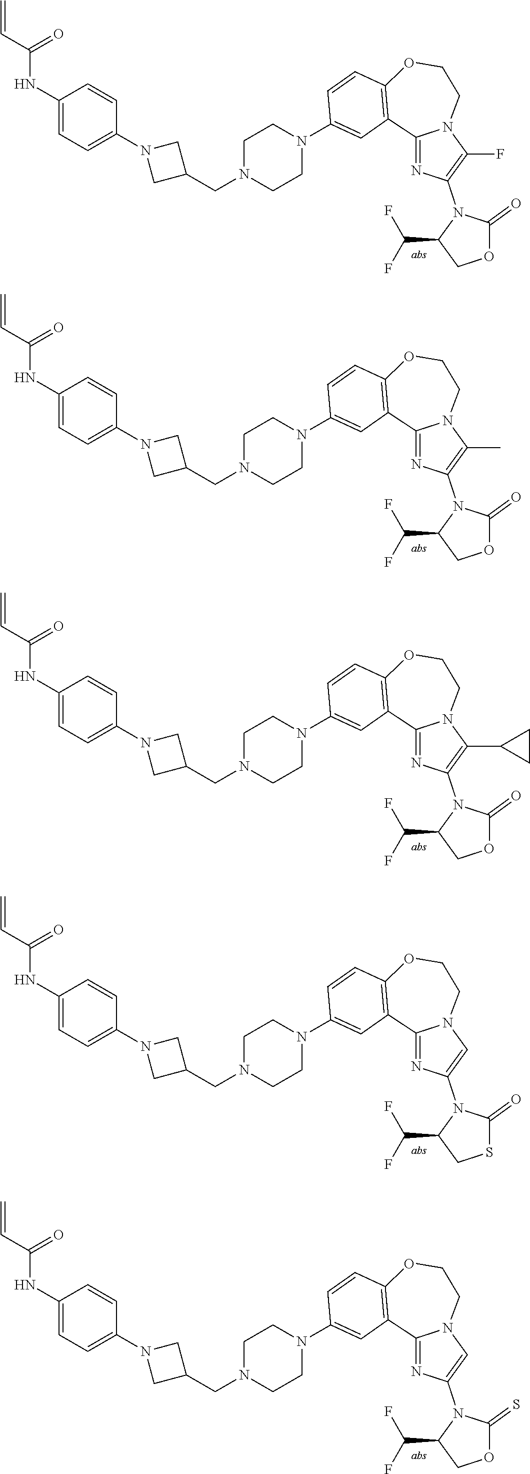 [SMILES: C=CC(=O)Nc1ccc(N2CC(CN3CCN(c4ccc5c(c4)-c4nc(N6C(=O)OC[C@H]6C(F)F)c(F)n4CCO5)CC3)C2)cc1 ; C=CC(=O)Nc1ccc(N2CC(CN3CCN(c4ccc5c(c4)-c4nc(N6C(=O)OC[C@H]6C(F)F)c(C)n4CCO5)CC3)C2)cc1 ; C=CC(=O)Nc1ccc(N2CC(CN3CCN(c4ccc5c(c4)-c4nc(N6C(=O)OC[C@H]6C(F)F)c(C6CC6)n4CCO5)CC3)C2)cc1 ; C=CC(=O)Nc1ccc(N2CC(CN3CCN(c4ccc5c(c4)-c4nc(N6C(=O)SC[C@H]6C(F)F)cn4CCO5)CC3)C2)cc1 ; C=CC(=O)Nc1ccc(N2CC(CN3CCN(c4ccc5c(c4)-c4nc(N6C(=S)OC[C@H]6C(F)F)cn4CCO5)CC3)C2)cc1]

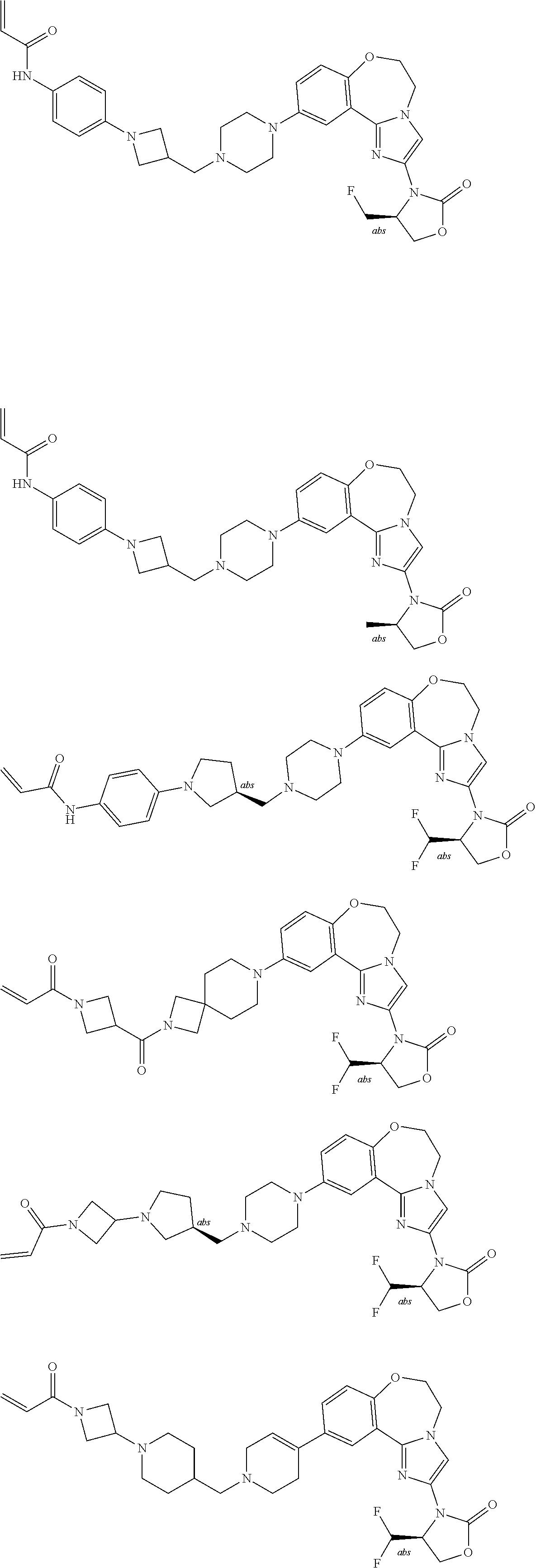

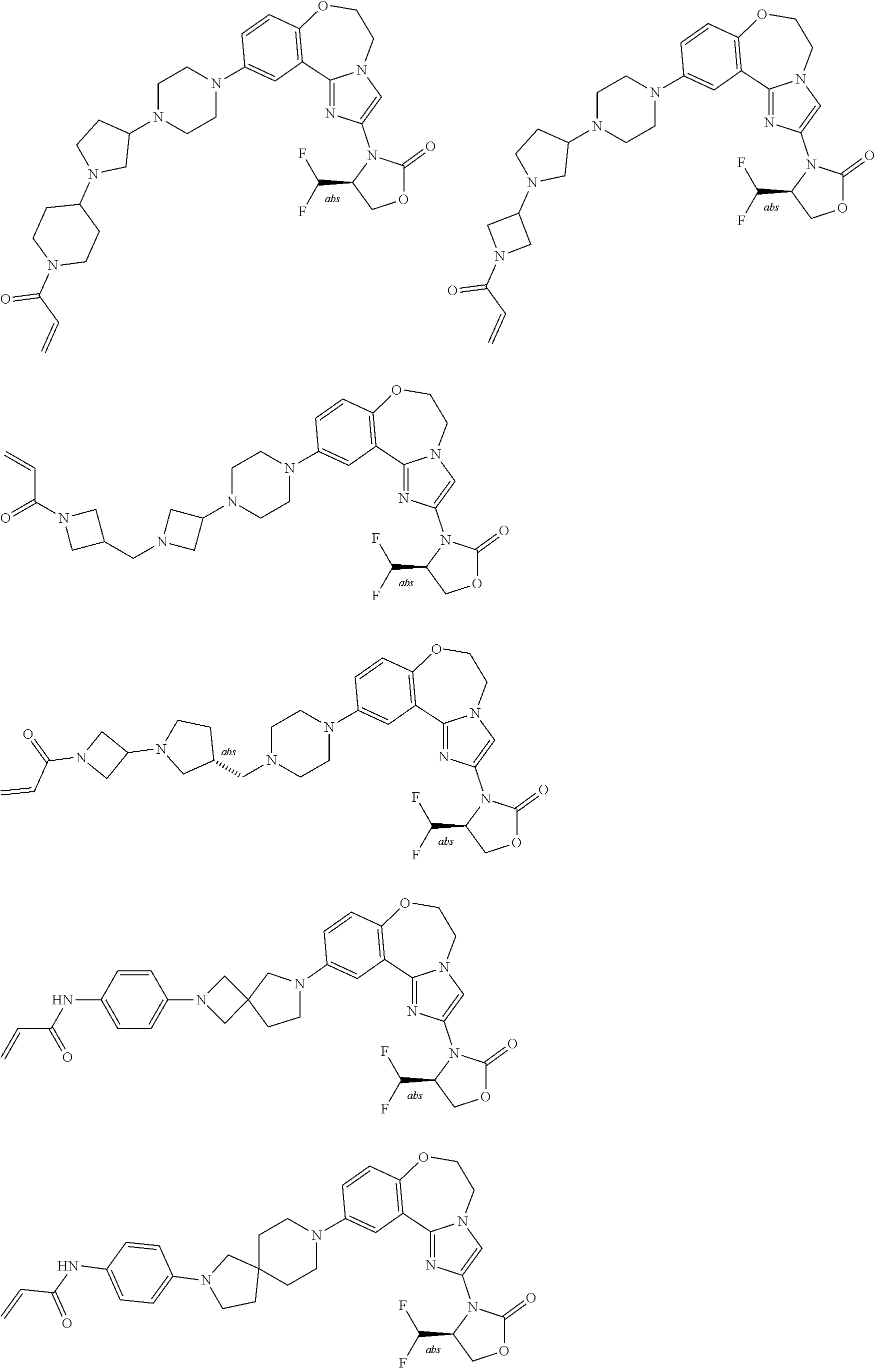

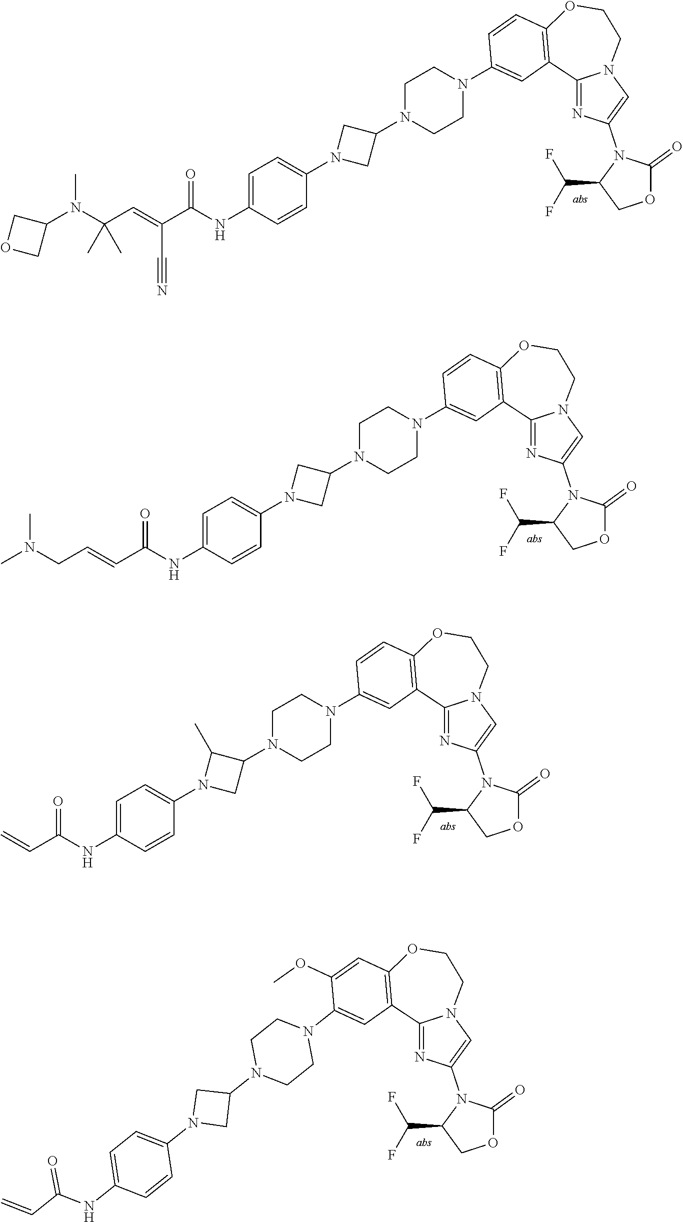 [SMILES: CN(C1COC1)C(C)(C)/C=C(\C#N)C(=O)Nc1ccc(N2CC(N3CCN(c4ccc5c(c4)-c4nc(N6C(=O)OC[C@H]6C(F)F)cn4CCO5)CC3)C2)cc1 ; CN(C)C/C=C/C(=O)Nc1ccc(N2CC(N3CCN(c4ccc5c(c4)-c4nc(N6C(=O)OC[C@H]6C(F)F)cn4CCO5)CC3)C2)cc1 ; C=CC(=O)Nc1ccc(N2CC(N3CCN(c4ccc5c(c4)-c4nc(N6C(=O)OC[C@H]6C(F)F)cn4CCO5)CC3)C2C)cc1 ; C=CC(=O)Nc1ccc(N2CC(N3CCN(c4cc5c(cc4OC)OCCn4cc(N6C(=O)OC[C@H]6C(F)F)nc4-5)CC3)C2)cc1]

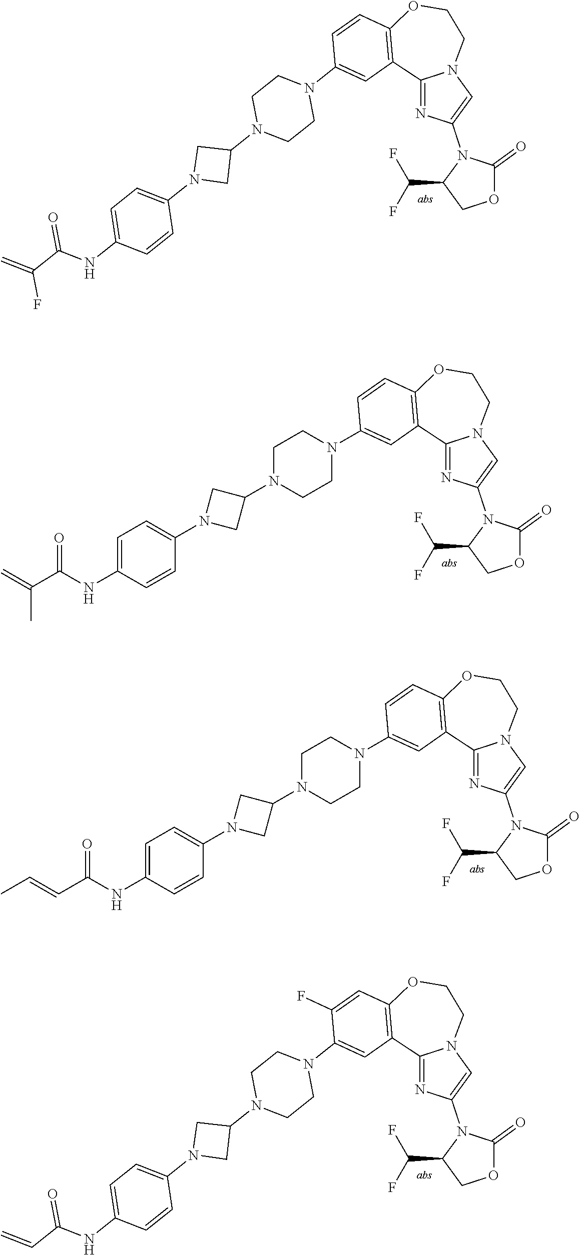 [SMILES: C=C(F)C(=O)Nc1ccc(N2CC(N3CCN(c4ccc5c(c4)-c4nc(N6C(=O)OC[C@H]6C(F)F)cn4CCO5)CC3)C2)cc1 ; C=C(C)C(=O)Nc1ccc(N2CC(N3CCN(c4ccc5c(c4)-c4nc(N6C(=O)OC[C@H]6C(F)F)cn4CCO5)CC3)C2)cc1 ; C/C=C/C(=O)Nc1ccc(N2CC(N3CCN(c4ccc5c(c4)-c4nc(N6C(=O)OC[C@H]6C(F)F)cn4CCO5)CC3)C2)cc1 ; C=CC(=O)Nc1ccc(N2CC(N3CCN(c4cc5c(cc4F)OCCn4cc(N6C(=O)OC[C@H]6C(F)F)nc4-5)CC3)C2)cc1]

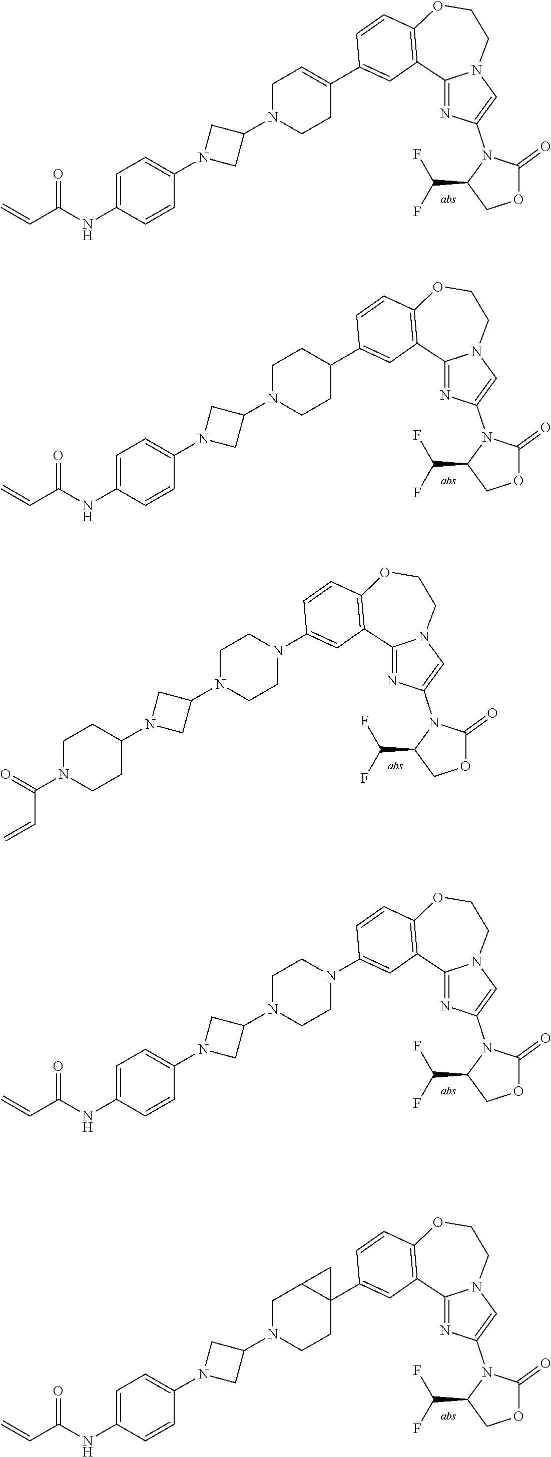 [SMILES: C=CC(=O)Nc1ccc(N2CC(N3CC=C(c4ccc5c(c4)-c4nc(N6C(=O)OC[C@H]6C(F)F)cn4CCO5)CC3)C2)cc1 ; C=CC(=O)Nc1ccc(N2CC(N3CCC(c4ccc5c(c4)-c4nc(N6C(=O)OC[C@H]6C(F)F)cn4CCO5)CC3)C2)cc1 ; C=CC(=O)N1CCC(N2CC(N3CCN(c4ccc5c(c4)-c4nc(N6C(=O)OC[C@H]6C(F)F)cn4CCO5)CC3)C2)CC1 ; C=CC(=O)Nc1ccc(N2CC(N3CCN(c4ccc5c(c4)-c4nc(N6C(=O)OC[C@H]6C(F)F)cn4CCO5)CC3)C2)cc1 ; C=CC(=O)Nc1ccc(N2CC(N3CCC4(c5ccc6c(c5)-c5nc(N7C(=O)OC[C@H]7C(F)F)cn5CCO6)CC4C3)C2)cc1]

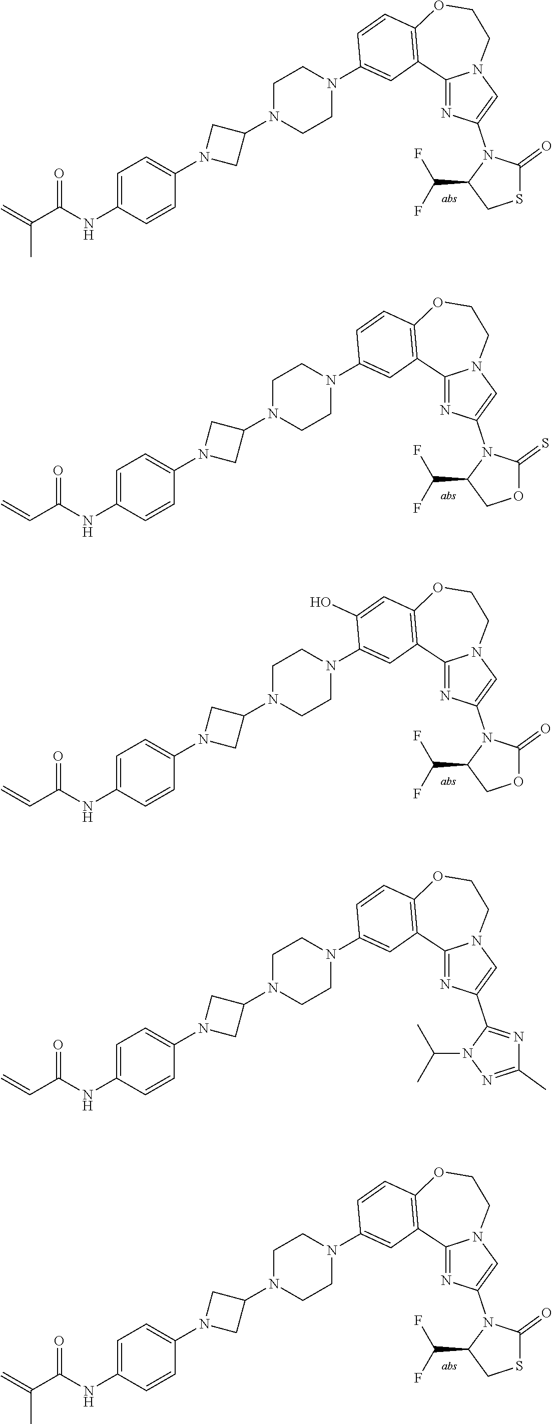

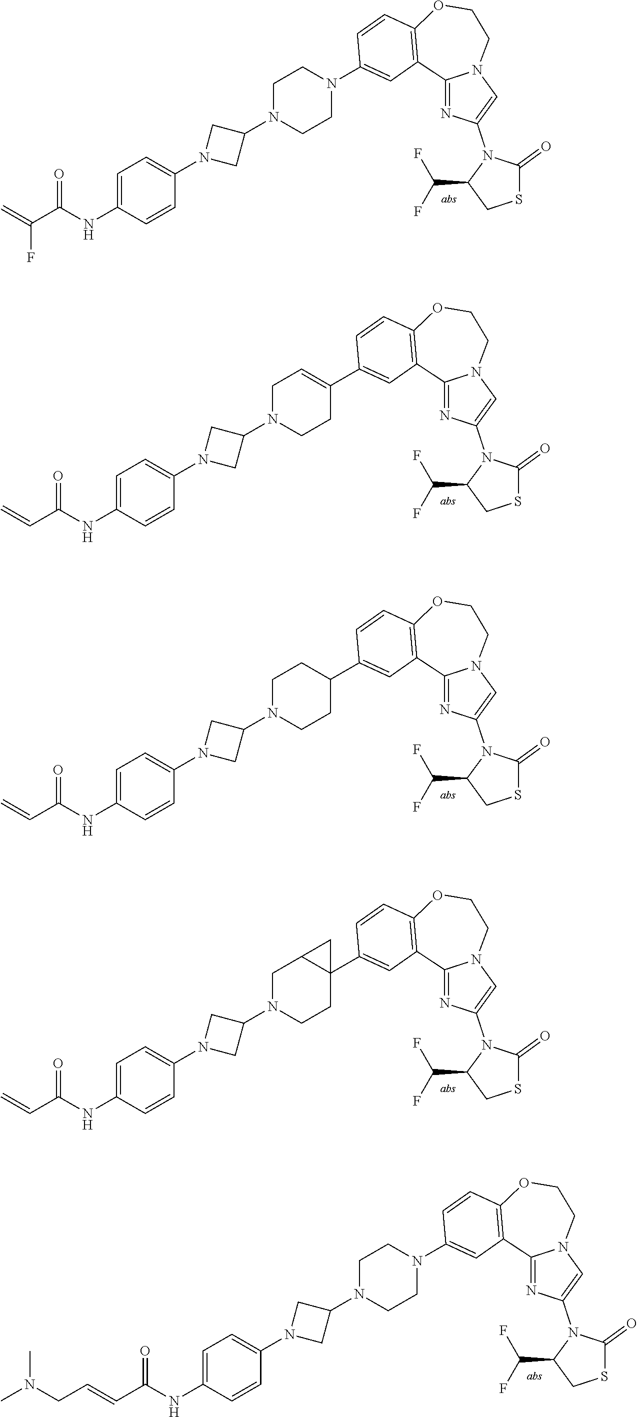 [SMILES: C=C(F)C(=O)Nc1ccc(N2CC(N3CCN(c4ccc5c(c4)-c4nc(N6C(=O)SC[C@H]6C(F)F)cn4CCO5)CC3)C2)cc1 ; C=CC(=O)Nc1ccc(N2CC(N3CC=C(c4ccc5c(c4)-c4nc(N6C(=O)SC[C@H]6C(F)F)cn4CCO5)CC3)C2)cc1 ; C=CC(=O)Nc1ccc(N2CC(N3CCC(c4ccc5c(c4)-c4nc(N6C(=O)SC[C@H]6C(F)F)cn4CCO5)CC3)C2)cc1 ; C=CC(=O)Nc1ccc(N2CC(N3CCC4(c5ccc6c(c5)-c5nc(N7C(=O)SC[C@H]7C(F)F)cn5CCO6)CC4C3)C2)cc1 ; CN(C)C/C=C/C(=O)Nc1ccc(N2CC(N3CCN(c4ccc5c(c4)-c4nc(N6C(=O)SC[C@H]6C(F)F)cn4CCO5)CC3)C2)cc1]

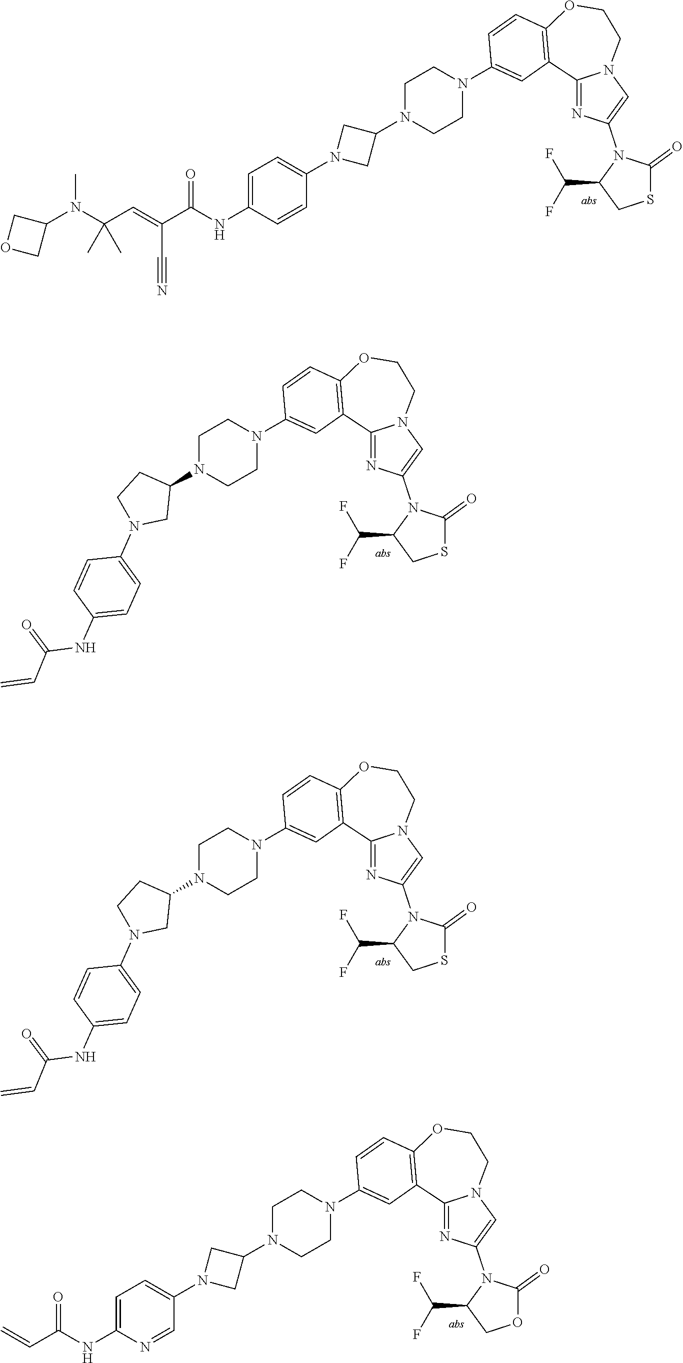 [SMILES: CN(C1COC1)C(C)(C)/C=C(\C#N)C(=O)Nc1ccc(N2CC(N3CCN(c4ccc5c(c4)-c4nc(N6C(=O)SC[C@H]6C(F)F)cn4CCO5)CC3)C2)cc1 ; C=CC(=O)Nc1ccc(N2CC[C@@H](N3CCN(c4ccc5c(c4)-c4nc(N6C(=O)SC[C@H]6C(F)F)cn4CCO5)CC3)C2)cc1 ; C=CC(=O)Nc1ccc(N2CC[C@H](N3CCN(c4ccc5c(c4)-c4nc(N6C(=O)SC[C@H]6C(F)F)cn4CCO5)CC3)C2)cc1 ; C=CC(=O)Nc1ccc(N2CC(N3CCN(c4ccc5c(c4)-c4nc(N6C(=O)OC[C@H]6C(F)F)cn4CCO5)CC3)C2)cn1]

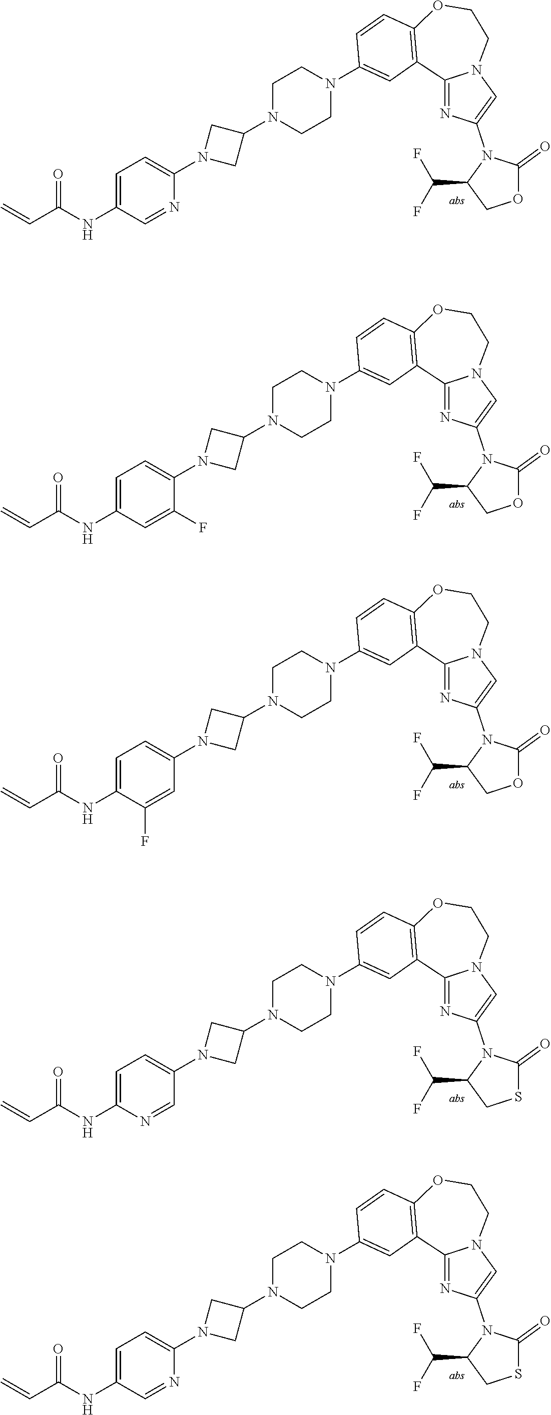 [SMILES: C=CC(=O)Nc1ccc(N2CC(N3CCN(c4ccc5c(c4)-c4nc(N6C(=O)OC[C@H]6C(F)F)cn4CCO5)CC3)C2)nc1 ; C=CC(=O)Nc1ccc(N2CC(N3CCN(c4ccc5c(c4)-c4nc(N6C(=O)OC[C@H]6C(F)F)cn4CCO5)CC3)C2)c(F)c1 ; C=CC(=O)Nc1ccc(N2CC(N3CCN(c4ccc5c(c4)-c4nc(N6C(=O)OC[C@H]6C(F)F)cn4CCO5)CC3)C2)cc1F ; C=CC(=O)Nc1ccc(N2CC(N3CCN(c4ccc5c(c4)-c4nc(N6C(=O)SC[C@H]6C(F)F)cn4CCO5)CC3)C2)cn1 ; C=CC(=O)Nc1ccc(N2CC(N3CCN(c4ccc5c(c4)-c4nc(N6C(=O)SC[C@H]6C(F)F)cn4CCO5)CC3)C2)nc1]

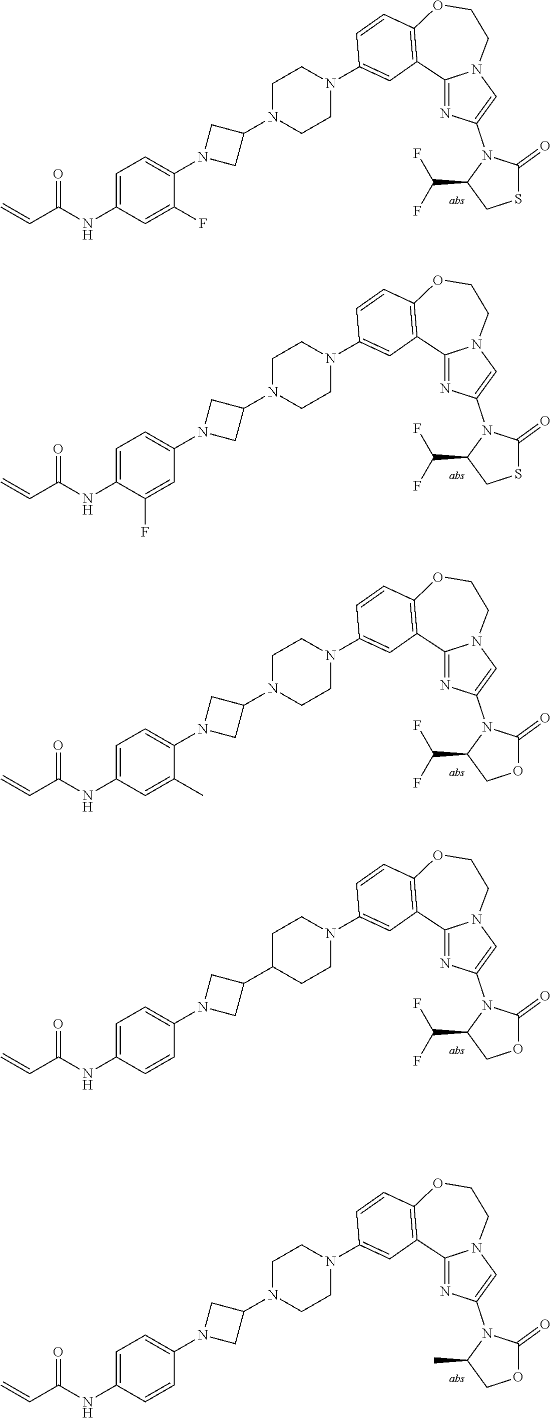 [SMILES: C=CC(=O)Nc1ccc(N2CC(N3CCN(c4ccc5c(c4)-c4nc(N6C(=O)SC[C@H]6C(F)F)cn4CCO5)CC3)C2)c(F)c1 ; C=CC(=O)Nc1ccc(N2CC(N3CCN(c4ccc5c(c4)-c4nc(N6C(=O)SC[C@H]6C(F)F)cn4CCO5)CC3)C2)cc1F ; C=CC(=O)Nc1ccc(N2CC(N3CCN(c4ccc5c(c4)-c4nc(N6C(=O)OC[C@H]6C(F)F)cn4CCO5)CC3)C2)c(C)c1 ; C=CC(=O)Nc1ccc(N2CC(C3CCN(c4ccc5c(c4)-c4nc(N6C(=O)OC[C@H]6C(F)F)cn4CCO5)CC3)C2)cc1 ; C=CC(=O)Nc1ccc(N2CC(N3CCN(c4ccc5c(c4)-c4nc(N6C(=O)OC[C@H]6C)cn4CCO5)CC3)C2)cc1]

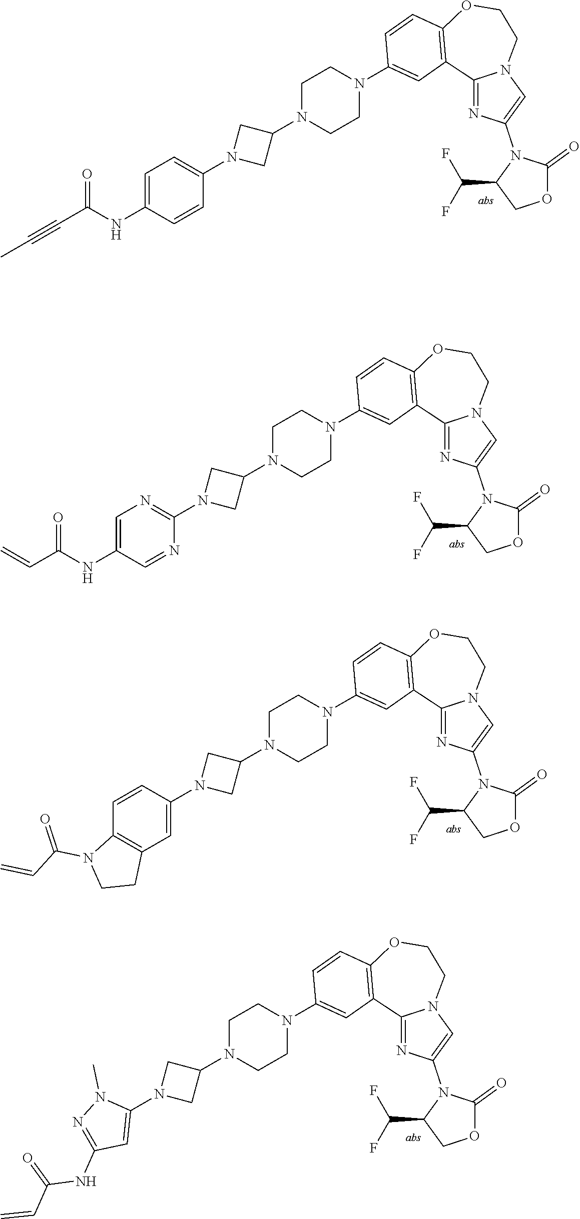 [SMILES: CC#CC(=O)Nc1ccc(N2CC(N3CCN(c4ccc5c(c4)-c4nc(N6C(=O)OC[C@H]6C(F)F)cn4CCO5)CC3)C2)cc1 ; C=CC(=O)Nc1cnc(N2CC(N3CCN(c4ccc5c(c4)-c4nc(N6C(=O)OC[C@H]6C(F)F)cn4CCO5)CC3)C2)nc1 ; C=CC(=O)N1CCc2cc(N3CC(N4CCN(c5ccc6c(c5)-c5nc(N7C(=O)OC[C@H]7C(F)F)cn5CCO6)CC4)C3)ccc21 ; C=CC(=O)Nc1cc(N2CC(N3CCN(c4ccc5c(c4)-c4nc(N6C(=O)OC[C@H]6C(F)F)cn4CCO5)CC3)C2)n(C)n1]

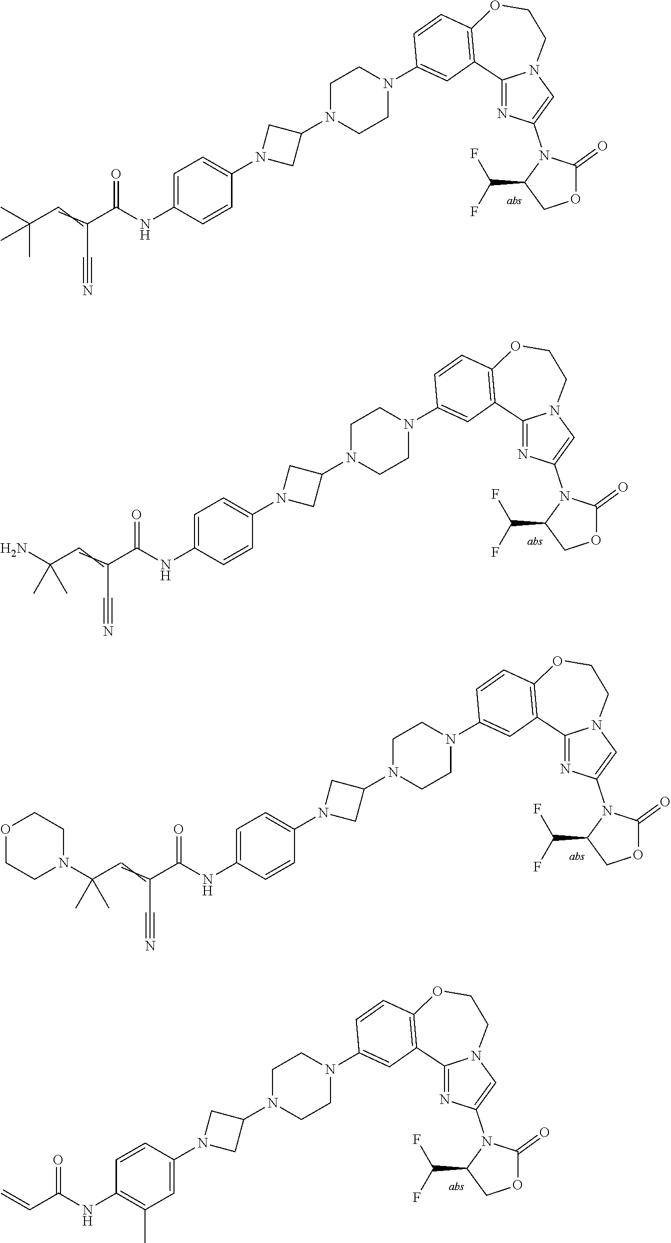 [SMILES: CC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1 ; CC(C)(N)C=C(C#N)C(=O)Nc1ccc(N2CC(N3CCN(c4ccc5c(c4)-c4nc(N6C(=O)OC[C@H]6C(F)F)cn4CCO5)CC3)C2)cc1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)N1CCOCC1 ; C=CC(=O)Nc1ccc(N2CC(N3CCN(c4ccc5c(c4)-c4nc(N6C(=O)OC[C@H]6C(F)F)cn4CCO5)CC3)C2)cc1C]

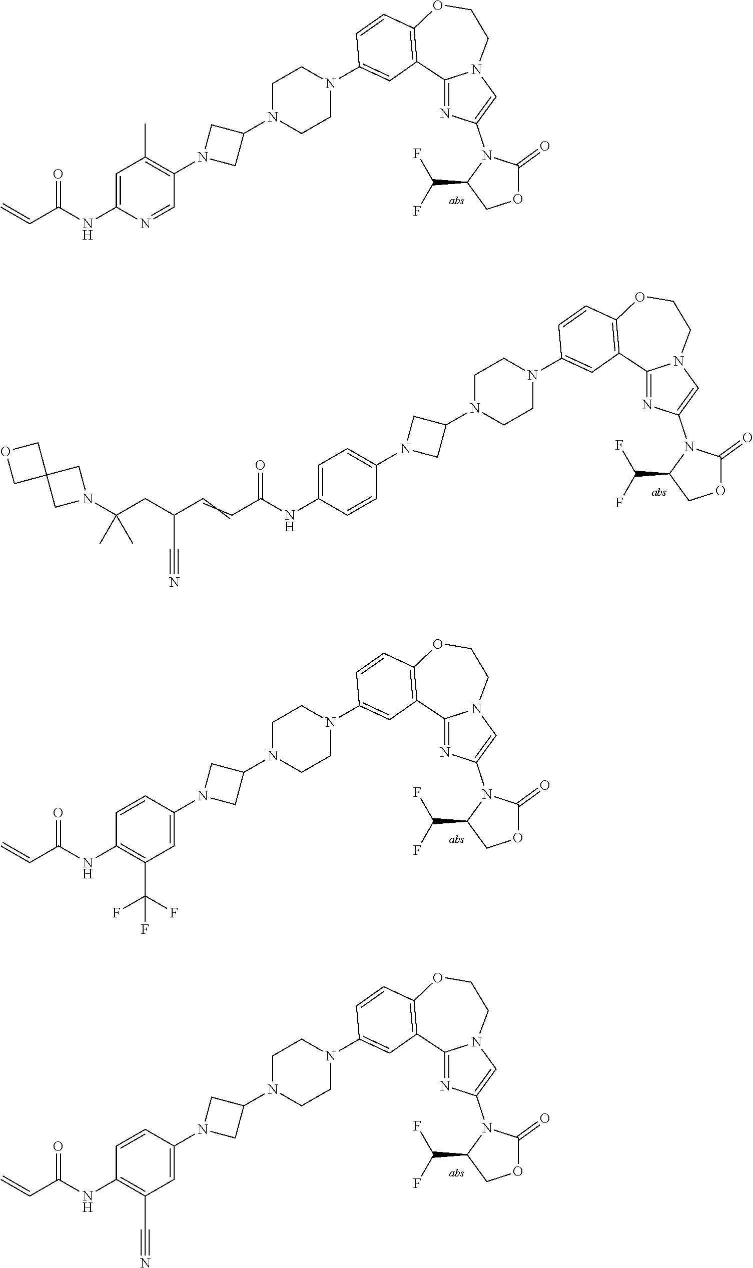 [SMILES: C=CC(=O)Nc1cc(C)c(N2CC(N3CCN(c4ccc5c(c4)-c4nc(N6C(=O)OC[C@H]6C(F)F)cn4CCO5)CC3)C2)cn1 ; CC(C)(CC(C#N)C=CC(=O)Nc1ccc(N2CC(N3CCN(c4ccc5c(c4)-c4nc(N6C(=O)OC[C@H]6C(F)F)cn4CCO5)CC3)C2)cc1)N1CC2(COC2)C1 ; C=CC(=O)Nc1ccc(N2CC(N3CCN(c4ccc5c(c4)-c4nc(N6C(=O)OC[C@H]6C(F)F)cn4CCO5)CC3)C2)cc1C(F)(F)F ; C=CC(=O)Nc1ccc(N2CC(N3CCN(c4ccc5c(c4)-c4nc(N6C(=O)OC[C@H]6C(F)F)cn4CCO5)CC3)C2)cc1C#N]

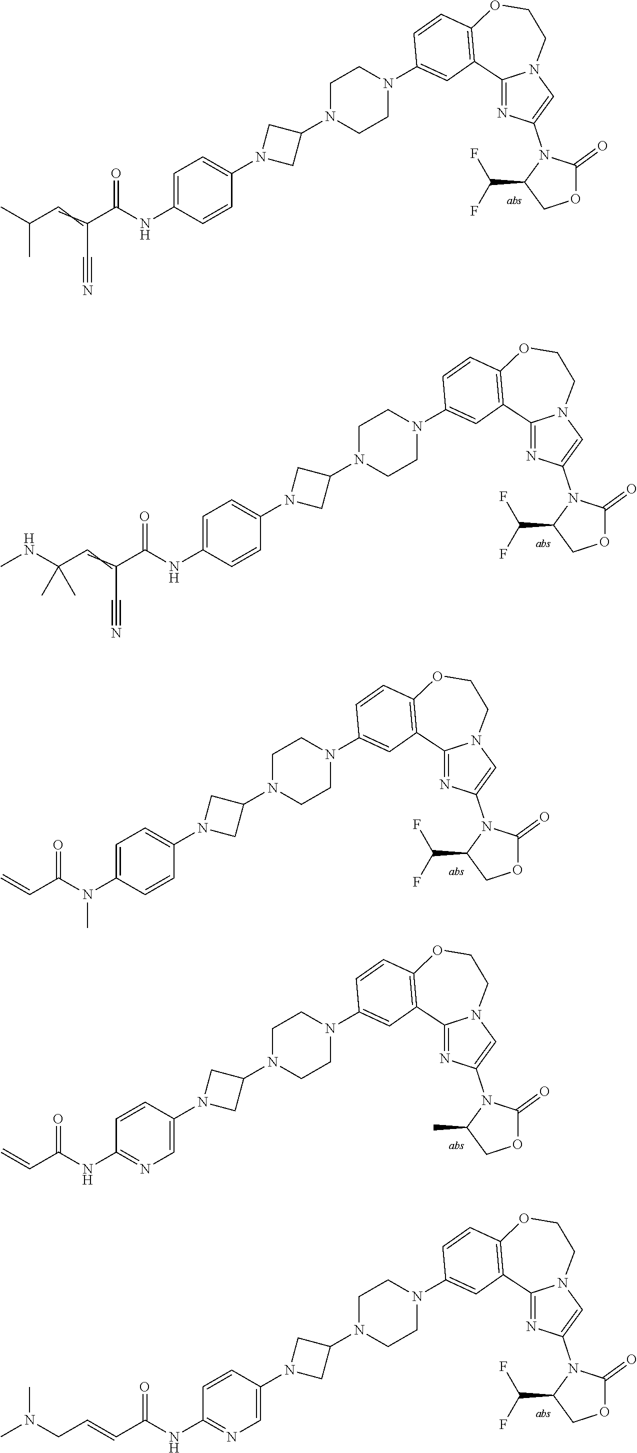 [SMILES: CC(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1 ; CNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1 ; C=CC(=O)N(C)c1ccc(N2CC(N3CCN(c4ccc5c(c4)-c4nc(N6C(=O)OC[C@H]6C(F)F)cn4CCO5)CC3)C2)cc1 ; C=CC(=O)Nc1ccc(N2CC(N3CCN(c4ccc5c(c4)-c4nc(N6C(=O)OC[C@H]6C)cn4CCO5)CC3)C2)cn1 ; CN(C)C/C=C/C(=O)Nc1ccc(N2CC(N3CCN(c4ccc5c(c4)-c4nc(N6C(=O)OC[C@H]6C(F)F)cn4CCO5)CC3)C2)cn1]

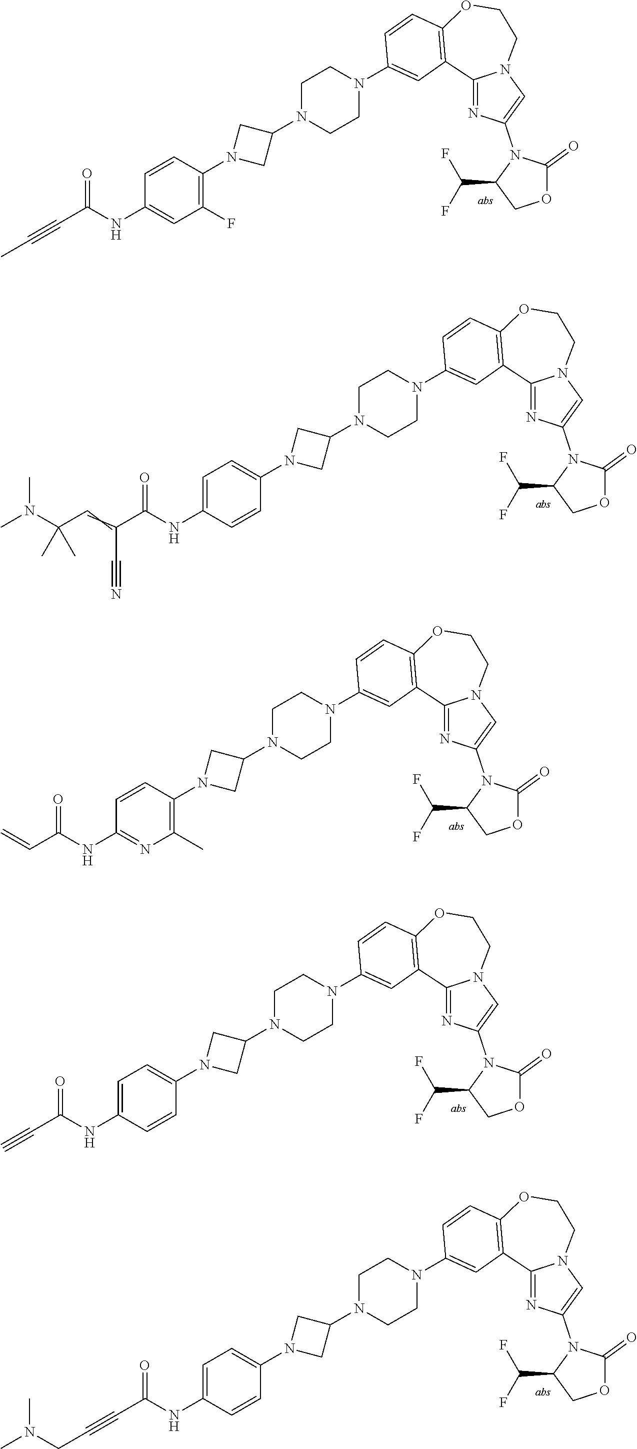 [SMILES: CC#CC(=O)Nc1ccc(N2CC(N3CCN(c4ccc5c(c4)-c4nc(N6C(=O)OC[C@H]6C(F)F)cn4CCO5)CC3)C2)c(F)c1 ; CN(C)C(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1 ; C=CC(=O)Nc1ccc(N2CC(N3CCN(c4ccc5c(c4)-c4nc(N6C(=O)OC[C@H]6C(F)F)cn4CCO5)CC3)C2)c(C)n1 ; C#CC(=O)Nc1ccc(N2CC(N3CCN(c4ccc5c(c4)-c4nc(N6C(=O)OC[C@H]6C(F)F)cn4CCO5)CC3)C2)cc1 ; CN(C)CC#CC(=O)Nc1ccc(N2CC(N3CCN(c4ccc5c(c4)-c4nc(N6C(=O)OC[C@H]6C(F)F)cn4CCO5)CC3)C2)cc1]

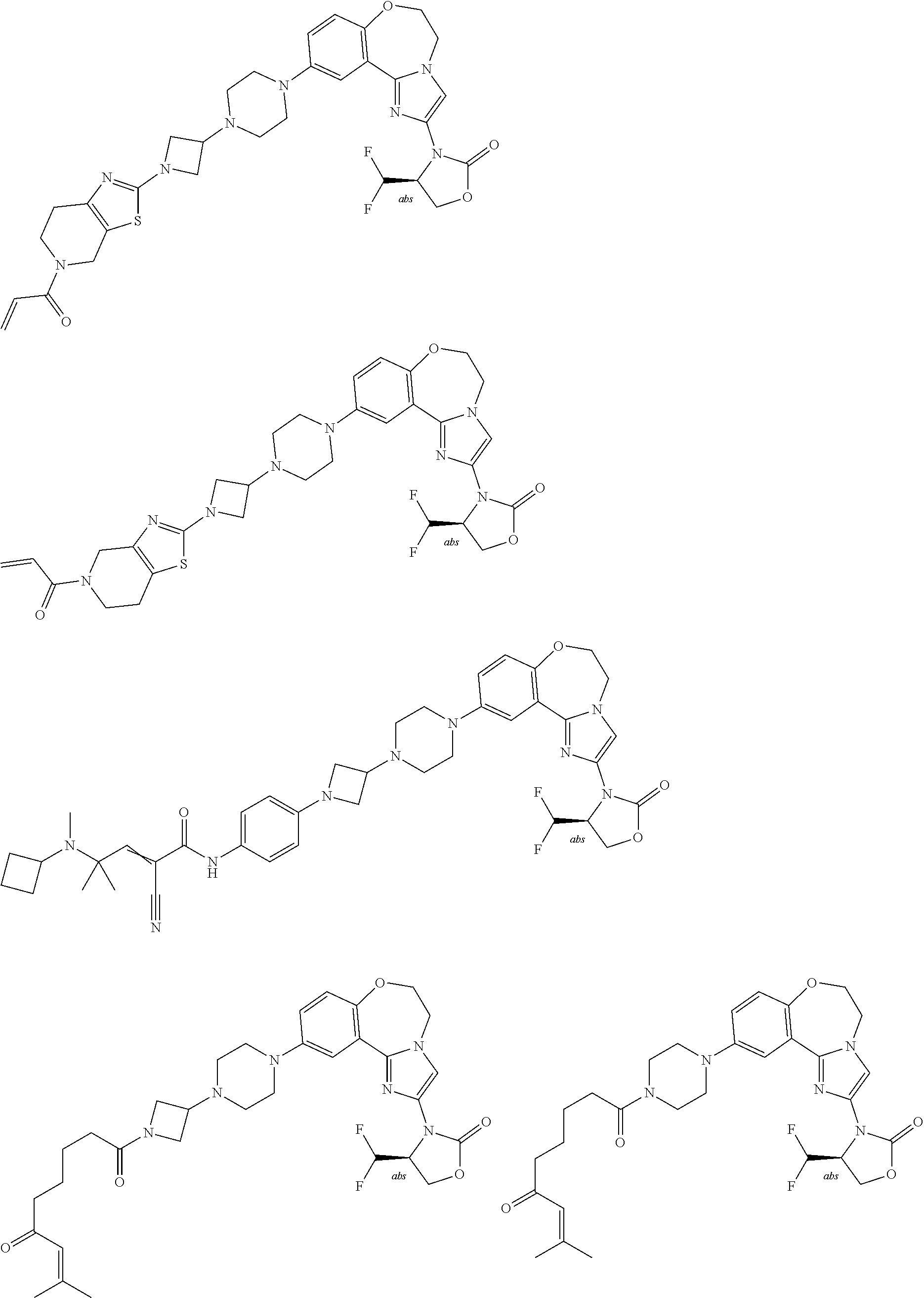 [SMILES: C=CC(=O)N1CCc2nc(N3CC(N4CCN(c5ccc6c(c5)-c5nc(N7C(=O)OC[C@H]7C(F)F)cn5CCO6)CC4)C3)sc2C1 ; C=CC(=O)N1CCc2sc(N3CC(N4CCN(c5ccc6c(c5)-c5nc(N7C(=O)OC[C@H]7C(F)F)cn5CCO6)CC4)C3)nc2C1 ; CN(C1CCC1)C(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1 ; CC(C)=CC(=O)CCCCC(=O)N1CC(N2CCN(c3ccc4c(c3)-c3nc(N5C(=O)OC[C@H]5C(F)F)cn3CCO4)CC2)C1 ; CC(C)=CC(=O)CCCCC(=O)N1CCN(c2ccc3c(c2)-c2nc(N4C(=O)OC[C@H]4C(F)F)cn2CCO3)CC1]

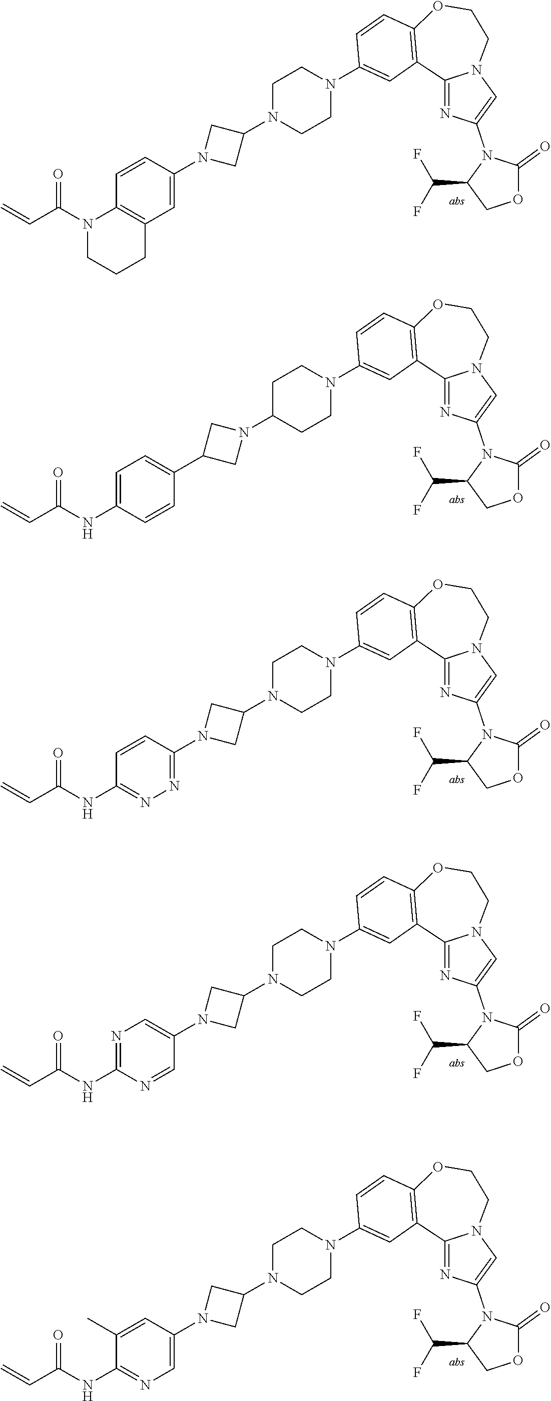 [SMILES: C=CC(=O)N1CCCc2cc(N3CC(N4CCN(c5ccc6c(c5)-c5nc(N7C(=O)OC[C@H]7C(F)F)cn5CCO6)CC4)C3)ccc21 ; C=CC(=O)Nc1ccc(C2CN(C3CCN(c4ccc5c(c4)-c4nc(N6C(=O)OC[C@H]6C(F)F)cn4CCO5)CC3)C2)cc1 ; C=CC(=O)Nc1ccc(N2CC(N3CCN(c4ccc5c(c4)-c4nc(N6C(=O)OC[C@H]6C(F)F)cn4CCO5)CC3)C2)nn1 ; C=CC(=O)Nc1ncc(N2CC(N3CCN(c4ccc5c(c4)-c4nc(N6C(=O)OC[C@H]6C(F)F)cn4CCO5)CC3)C2)cn1 ; C=CC(=O)Nc1ncc(N2CC(N3CCN(c4ccc5c(c4)-c4nc(N6C(=O)OC[C@H]6C(F)F)cn4CCO5)CC3)C2)cc1C]

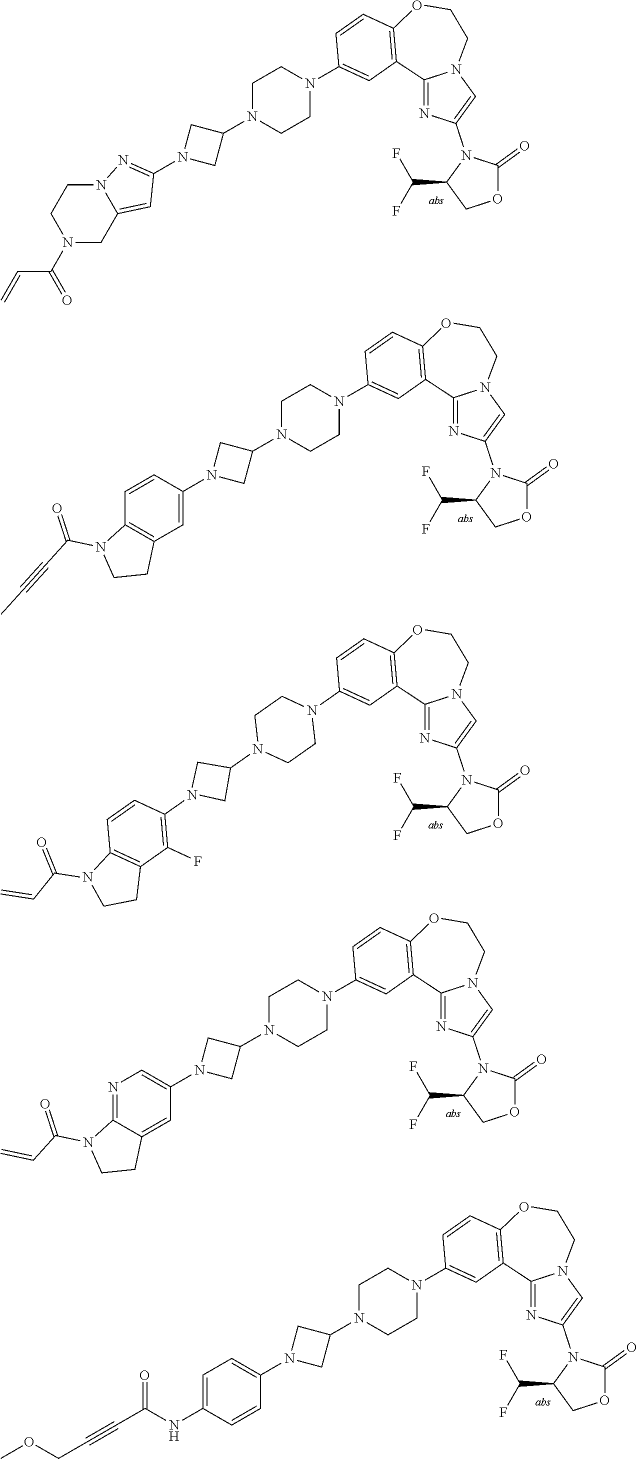 [SMILES: C=CC(=O)N1CCn2nc(N3CC(N4CCN(c5ccc6c(c5)-c5nc(N7C(=O)OC[C@H]7C(F)F)cn5CCO6)CC4)C3)cc2C1 ; CC#CC(=O)N1CCc2cc(N3CC(N4CCN(c5ccc6c(c5)-c5nc(N7C(=O)OC[C@H]7C(F)F)cn5CCO6)CC4)C3)ccc21 ; C=CC(=O)N1CCc2c1ccc(N1CC(N3CCN(c4ccc5c(c4)-c4nc(N6C(=O)OC[C@H]6C(F)F)cn4CCO5)CC3)C1)c2F ; C=CC(=O)N1CCc2cc(N3CC(N4CCN(c5ccc6c(c5)-c5nc(N7C(=O)OC[C@H]7C(F)F)cn5CCO6)CC4)C3)cnc21 ; COCC#CC(=O)Nc1ccc(N2CC(N3CCN(c4ccc5c(c4)-c4nc(N6C(=O)OC[C@H]6C(F)F)cn4CCO5)CC3)C2)cc1]

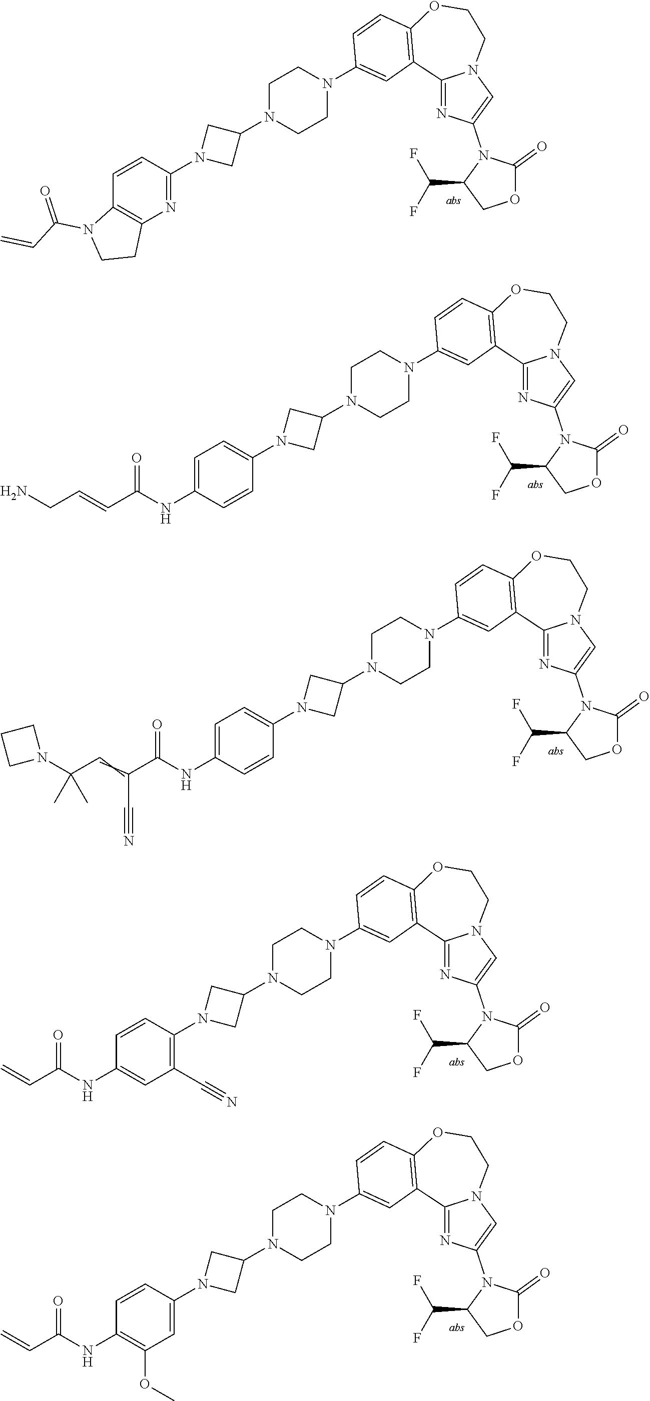 [SMILES: C=CC(=O)N1CCc2nc(N3CC(N4CCN(c5ccc6c(c5)-c5nc(N7C(=O)OC[C@H]7C(F)F)cn5CCO6)CC4)C3)ccc21 ; NC/C=C/C(=O)Nc1ccc(N2CC(N3CCN(c4ccc5c(c4)-c4nc(N6C(=O)OC[C@H]6C(F)F)cn4CCO5)CC3)C2)cc1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)N1CCC1 ; C=CC(=O)Nc1ccc(N2CC(N3CCN(c4ccc5c(c4)-c4nc(N6C(=O)OC[C@H]6C(F)F)cn4CCO5)CC3)C2)c(C#N)c1 ; C=CC(=O)Nc1ccc(N2CC(N3CCN(c4ccc5c(c4)-c4nc(N6C(=O)OC[C@H]6C(F)F)cn4CCO5)CC3)C2)cc1OC]

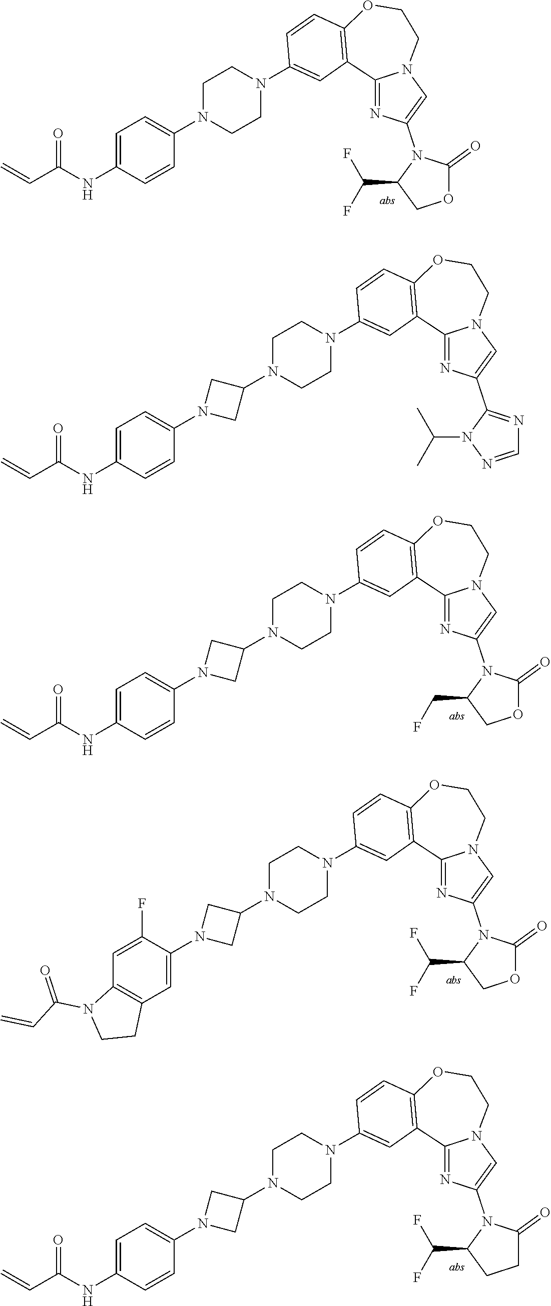 [SMILES: C=CC(=O)Nc1ccc(N2CCN(c3ccc4c(c3)-c3nc(N5C(=O)OC[C@H]5C(F)F)cn3CCO4)CC2)cc1 ; C=CC(=O)Nc1ccc(N2CC(N3CCN(c4ccc5c(c4)-c4nc(-c6ncnn6C(C)C)cn4CCO5)CC3)C2)cc1 ; C=CC(=O)Nc1ccc(N2CC(N3CCN(c4ccc5c(c4)-c4nc(N6C(=O)OC[C@H]6CF)cn4CCO5)CC3)C2)cc1 ; C=CC(=O)N1CCc2cc(N3CC(N4CCN(c5ccc6c(c5)-c5nc(N7C(=O)OC[C@H]7C(F)F)cn5CCO6)CC4)C3)c(F)cc21 ; C=CC(=O)Nc1ccc(N2CC(N3CCN(c4ccc5c(c4)-c4nc(N6C(=O)CC[C@H]6C(F)F)cn4CCO5)CC3)C2)cc1]

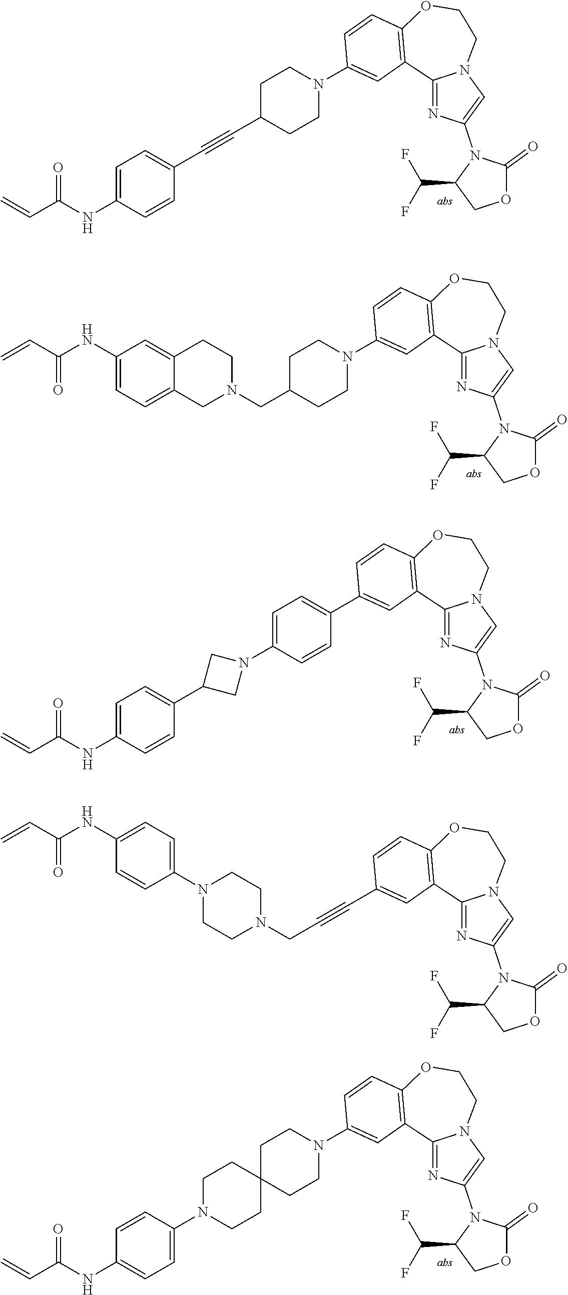

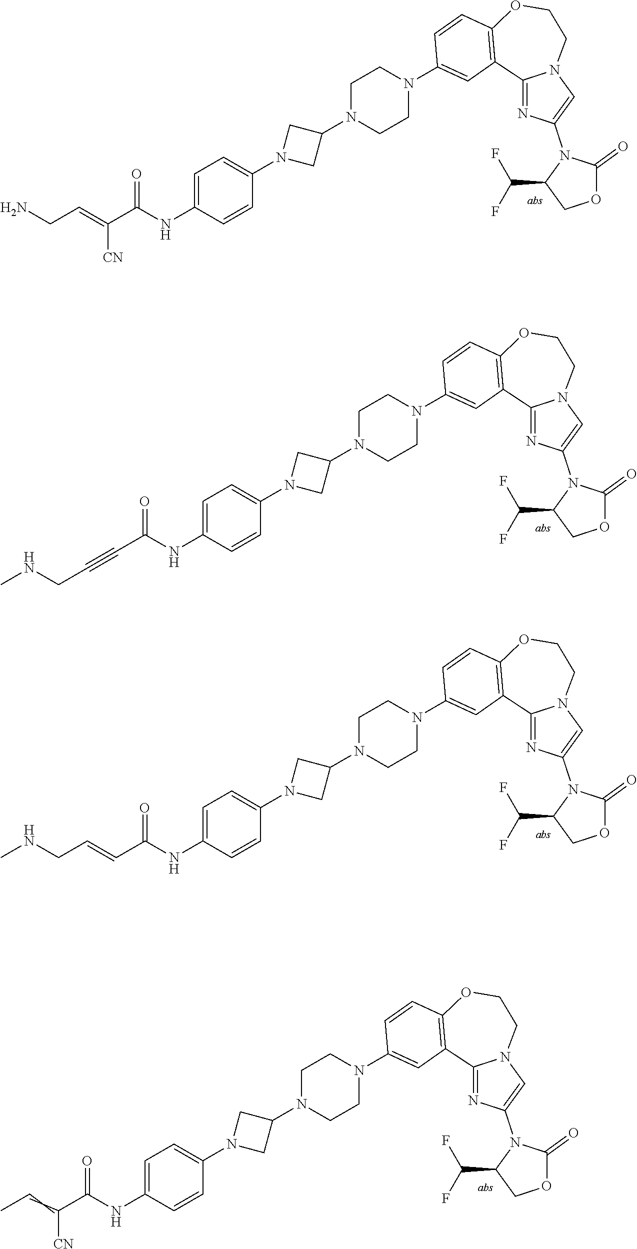 [SMILES: N#C/C(=C\CN)C(=O)Nc1ccc(N2CC(N3CCN(c4ccc5c(c4)-c4nc(N6C(=O)OC[C@H]6C(F)F)cn4CCO5)CC3)C2)cc1 ; CNCC#CC(=O)Nc1ccc(N2CC(N3CCN(c4ccc5c(c4)-c4nc(N6C(=O)OC[C@H]6C(F)F)cn4CCO5)CC3)C2)cc1 ; CNC/C=C/C(=O)Nc1ccc(N2CC(N3CCN(c4ccc5c(c4)-c4nc(N6C(=O)OC[C@H]6C(F)F)cn4CCO5)CC3)C2)cc1 ; CC=C(C#N)C(=O)Nc1ccc(N2CC(N3CCN(c4ccc5c(c4)-c4nc(N6C(=O)OC[C@H]6C(F)F)cn4CCO5)CC3)C2)cc1]

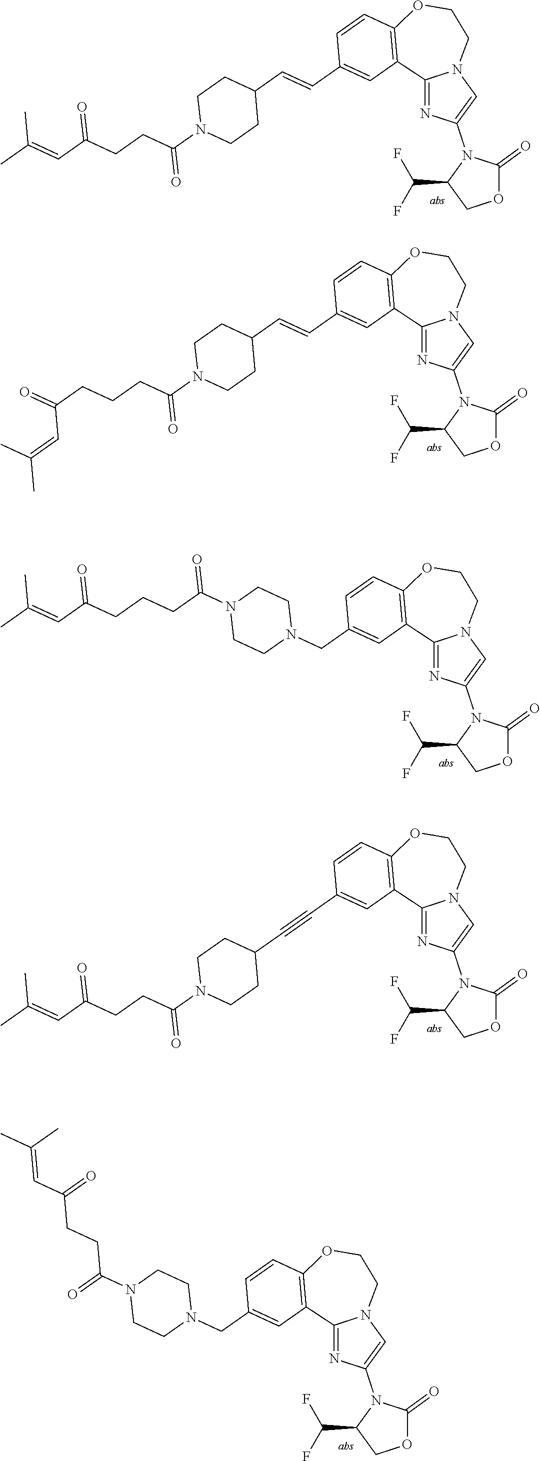

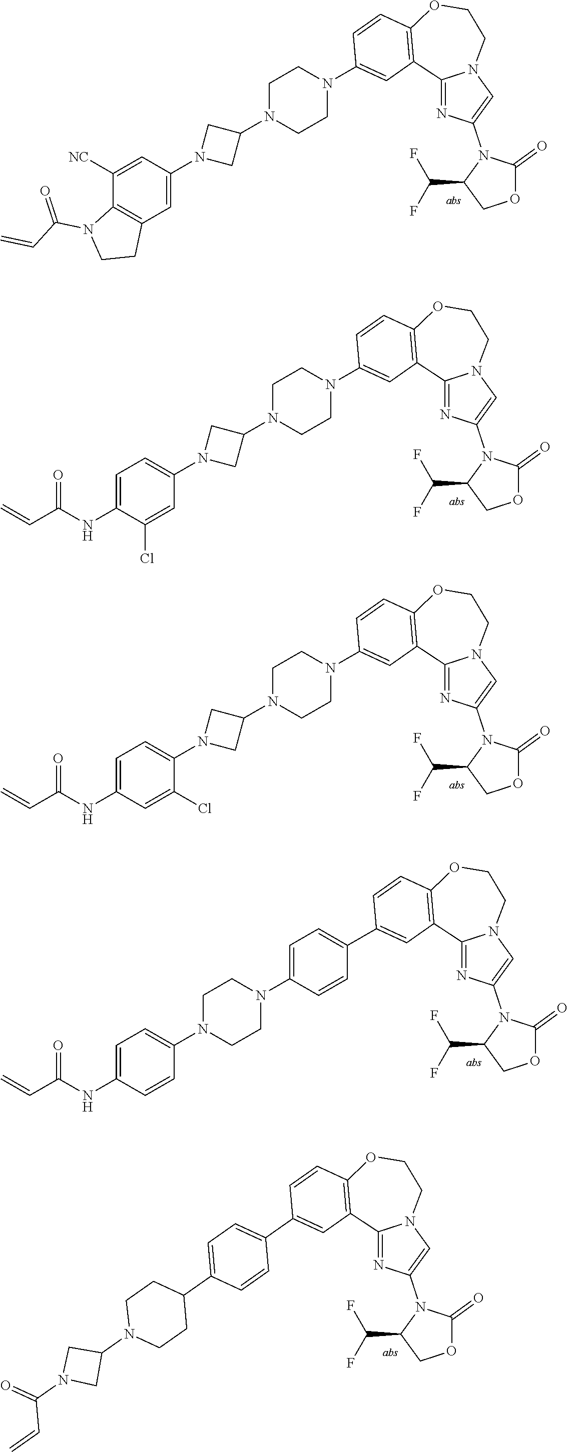 [SMILES: C=CC(=O)N1CCc2cc(N3CC(N4CCN(c5ccc6c(c5)-c5nc(N7C(=O)OC[C@H]7C(F)F)cn5CCO6)CC4)C3)cc(C#N)c21 ; C=CC(=O)Nc1ccc(N2CC(N3CCN(c4ccc5c(c4)-c4nc(N6C(=O)OC[C@H]6C(F)F)cn4CCO5)CC3)C2)cc1Cl ; C=CC(=O)Nc1ccc(N2CC(N3CCN(c4ccc5c(c4)-c4nc(N6C(=O)OC[C@H]6C(F)F)cn4CCO5)CC3)C2)c(Cl)c1 ; C=CC(=O)Nc1ccc(N2CCN(c3ccc(-c4ccc5c(c4)-c4nc(N6C(=O)OC[C@H]6C(F)F)cn4CCO5)cc3)CC2)cc1 ; C=CC(=O)N1CC(N2CCC(c3ccc(-c4ccc5c(c4)-c4nc(N6C(=O)OC[C@H]6C(F)F)cn4CCO5)cc3)CC2)C1]

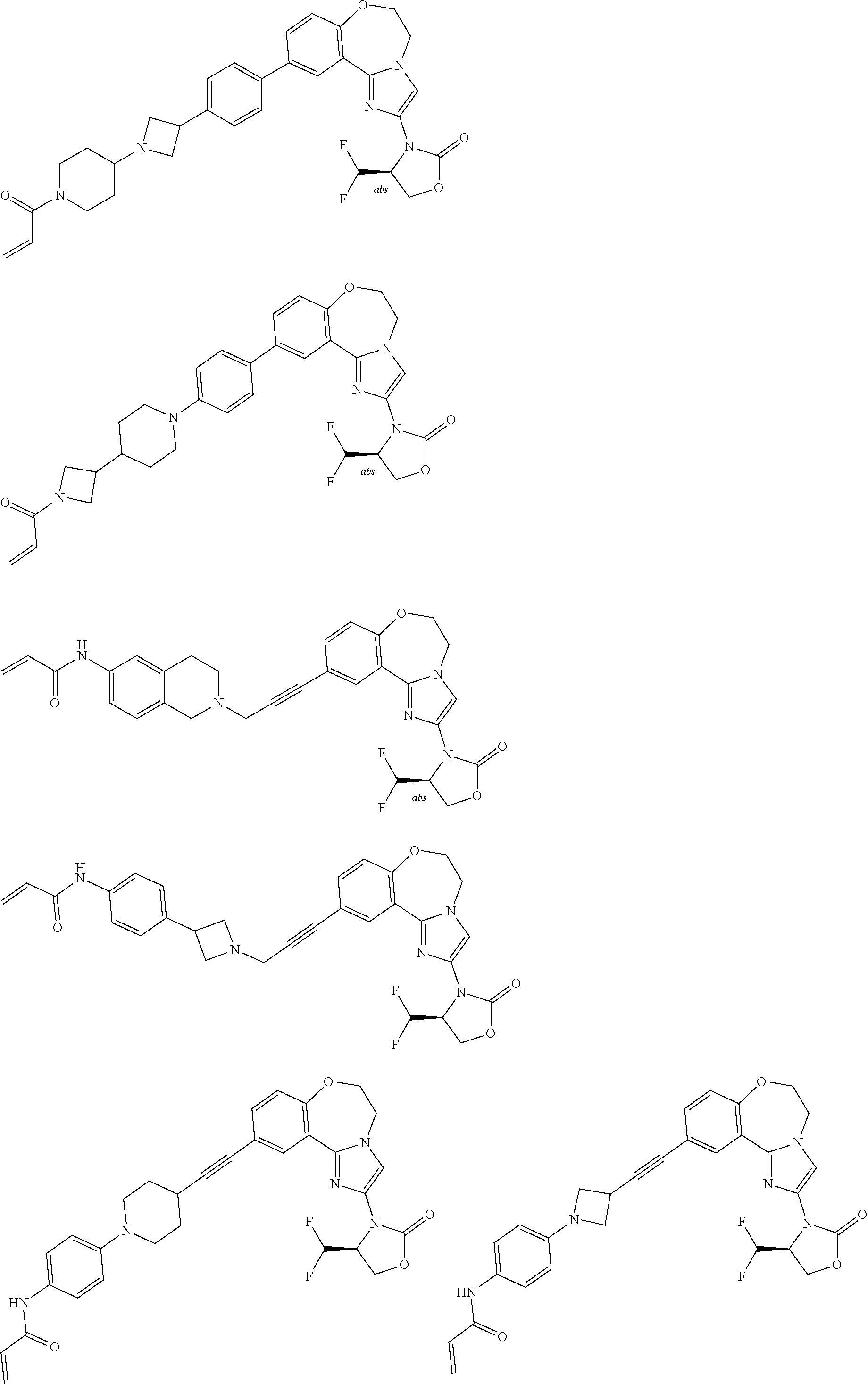

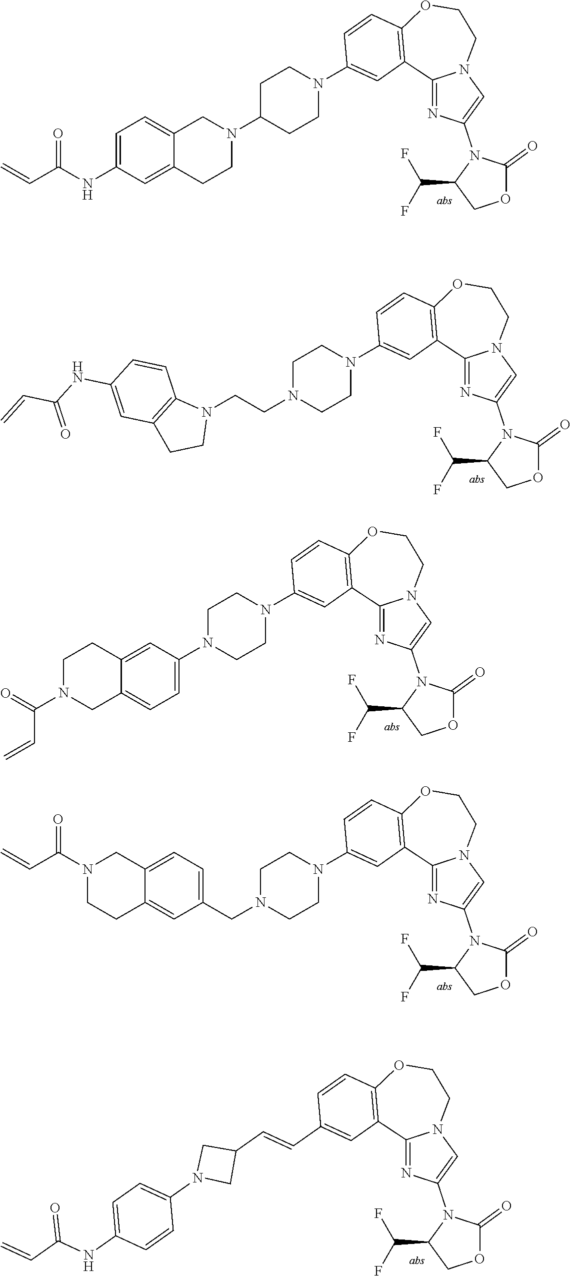 [SMILES: C=CC(=O)Nc1ccc2c(c1)CCN(C1CCN(c3ccc4c(c3)-c3nc(N5C(=O)OC[C@H]5C(F)F)cn3CCO4)CC1)C2 ; C=CC(=O)Nc1ccc2c(c1)CCN2CCN1CCN(c2ccc3c(c2)-c2nc(N4C(=O)OC[C@H]4C(F)F)cn2CCO3)CC1 ; C=CC(=O)N1CCc2cc(N3CCN(c4ccc5c(c4)-c4nc(N6C(=O)OC[C@H]6C(F)F)cn4CCO5)CC3)ccc2C1 ; C=CC(=O)N1CCc2cc(CN3CCN(c4ccc5c(c4)-c4nc(N6C(=O)OC[C@H]6C(F)F)cn4CCO5)CC3)ccc2C1 ; C=CC(=O)Nc1ccc(N2CC(/C=C/c3ccc4c(c3)-c3nc(N5C(=O)OC[C@H]5C(F)F)cn3CCO4)C2)cc1]

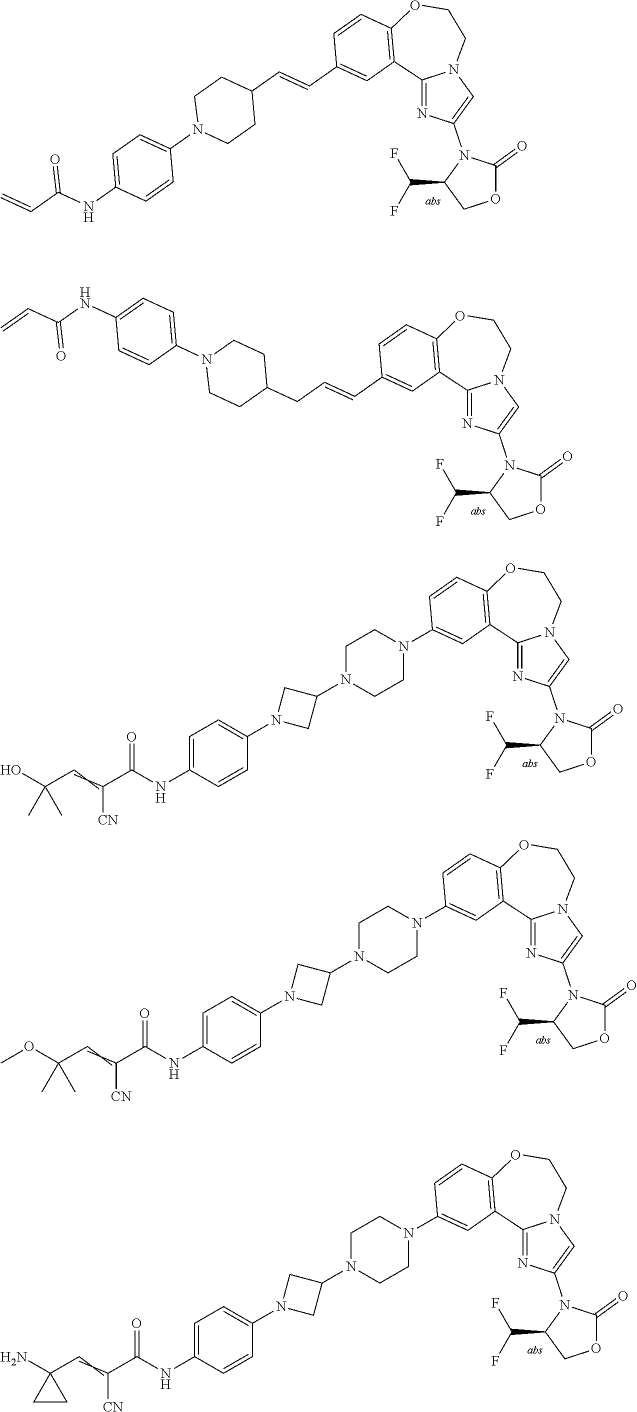 [SMILES: C=CC(=O)Nc1ccc(N2CCC(/C=C/c3ccc4c(c3)-c3nc(N5C(=O)OC[C@H]5C(F)F)cn3CCO4)CC2)cc1 ; C=CC(=O)Nc1ccc(N2CCC(C/C=C/c3ccc4c(c3)-c3nc(N5C(=O)OC[C@H]5C(F)F)cn3CCO4)CC2)cc1 ; CC(C)(O)C=C(C#N)C(=O)Nc1ccc(N2CC(N3CCN(c4ccc5c(c4)-c4nc(N6C(=O)OC[C@H]6C(F)F)cn4CCO5)CC3)C2)cc1 ; COC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1 ; N#CC(=CC1(N)CC1)C(=O)Nc1ccc(N2CC(N3CCN(c4ccc5c(c4)-c4nc(N6C(=O)OC[C@H]6C(F)F)cn4CCO5)CC3)C2)cc1]

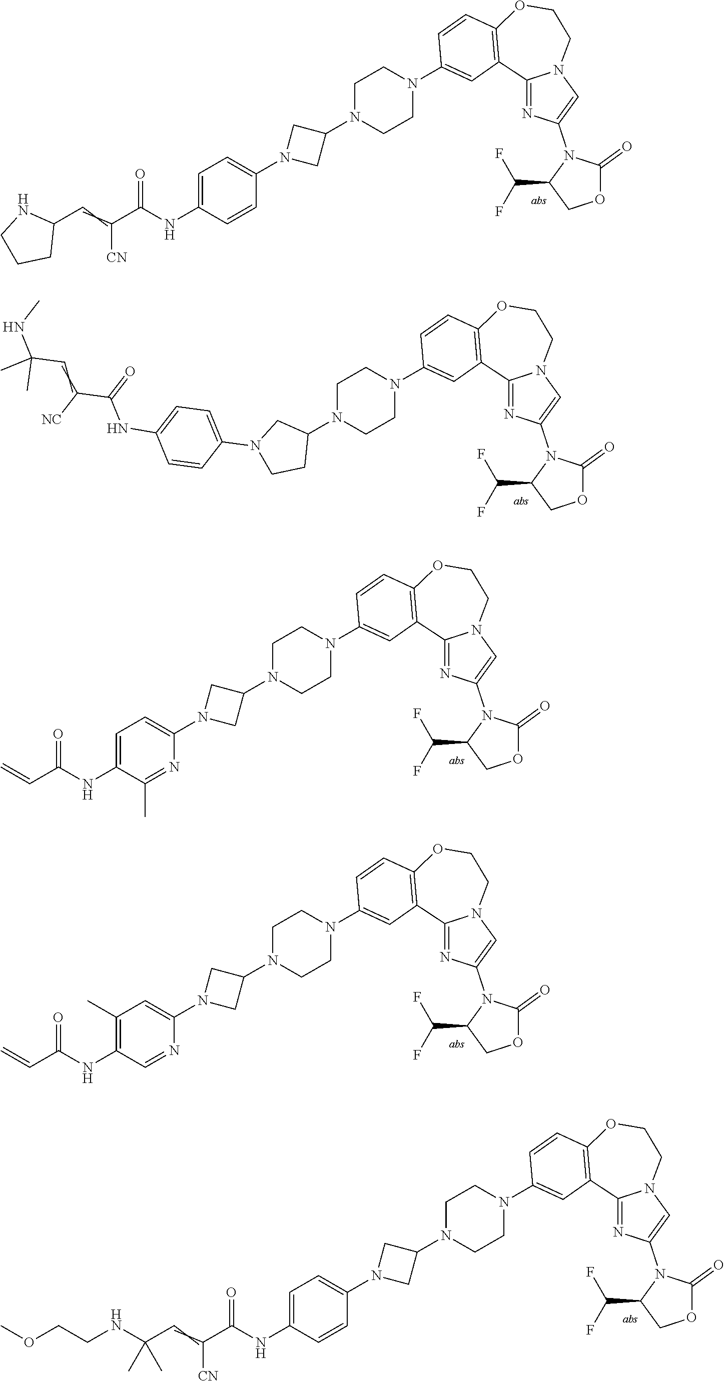

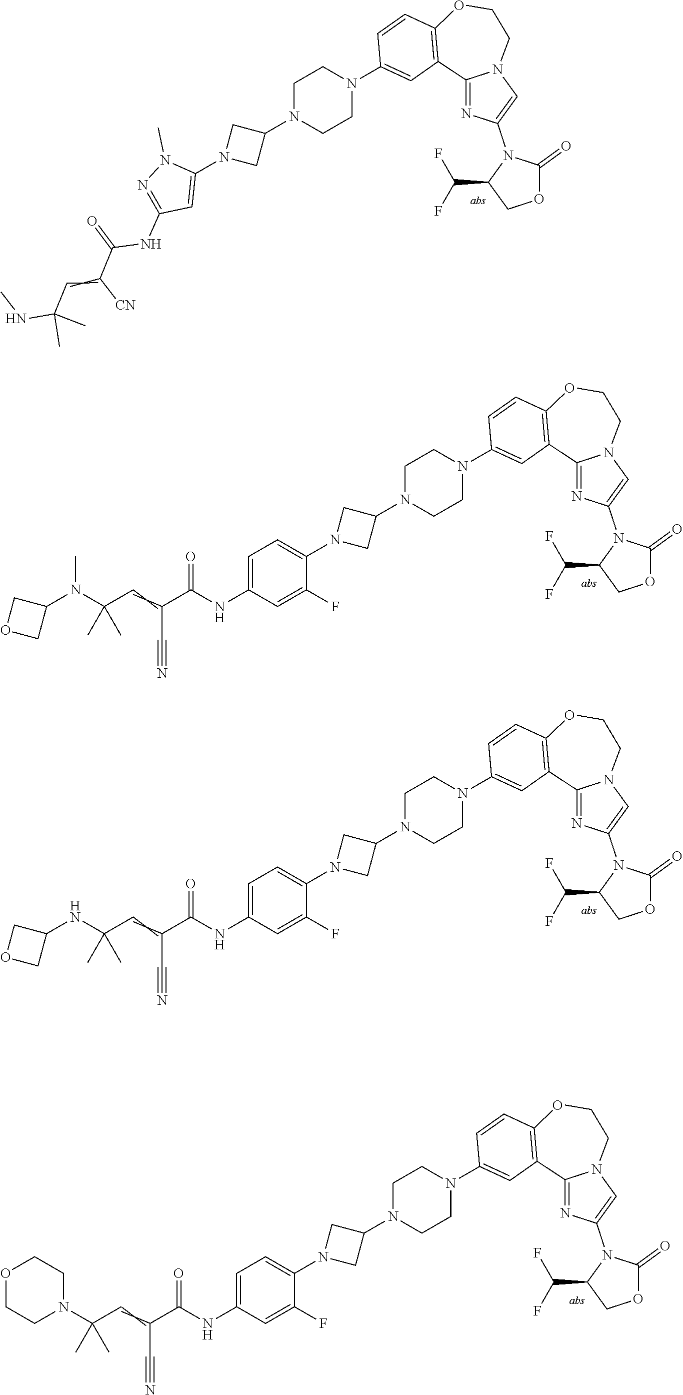 [SMILES: CNC(C)(C)C=C(C#N)C(=O)Nc1cc(N2CC(N3CCN(c4ccc5c(c4)-c4nc(N6C(=O)OC[C@H]6C(F)F)cn4CCO5)CC3)C2)n(C)n1 ; CN(C1COC1)C(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1)NC1COC1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1)N1CCOCC1]

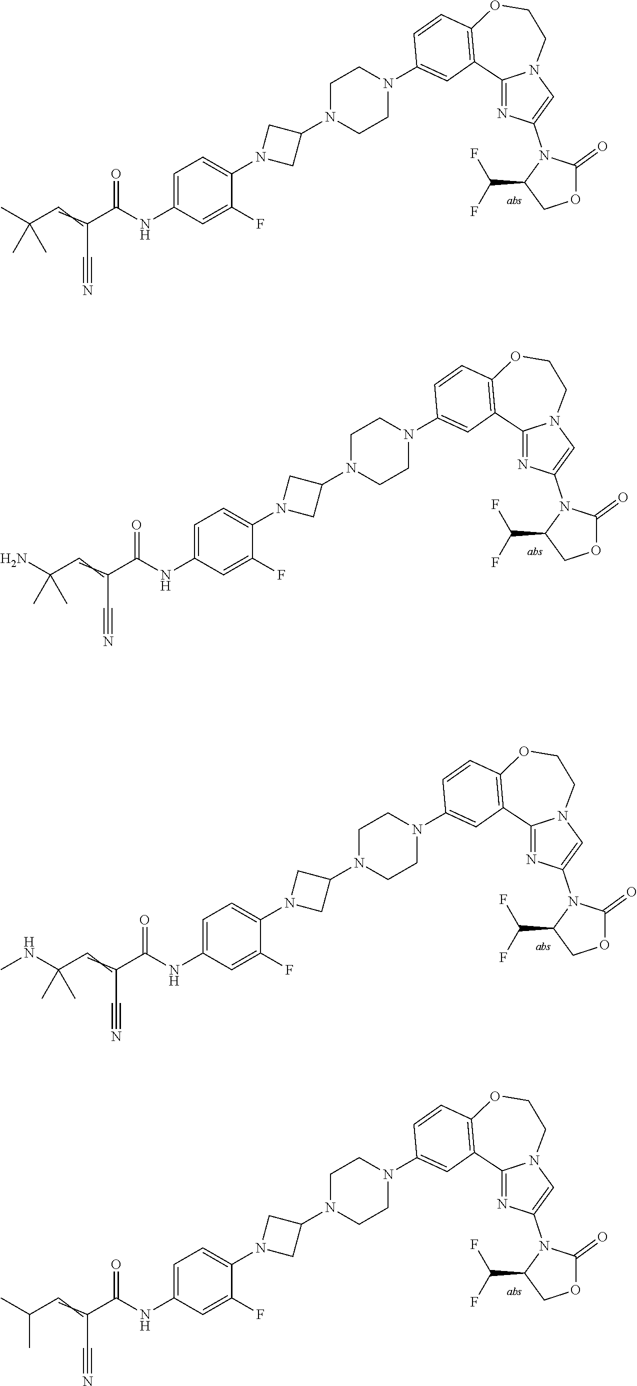 [SMILES: CC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1 ; CC(C)(N)C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1 ; CNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1 ; CC(C)C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1]

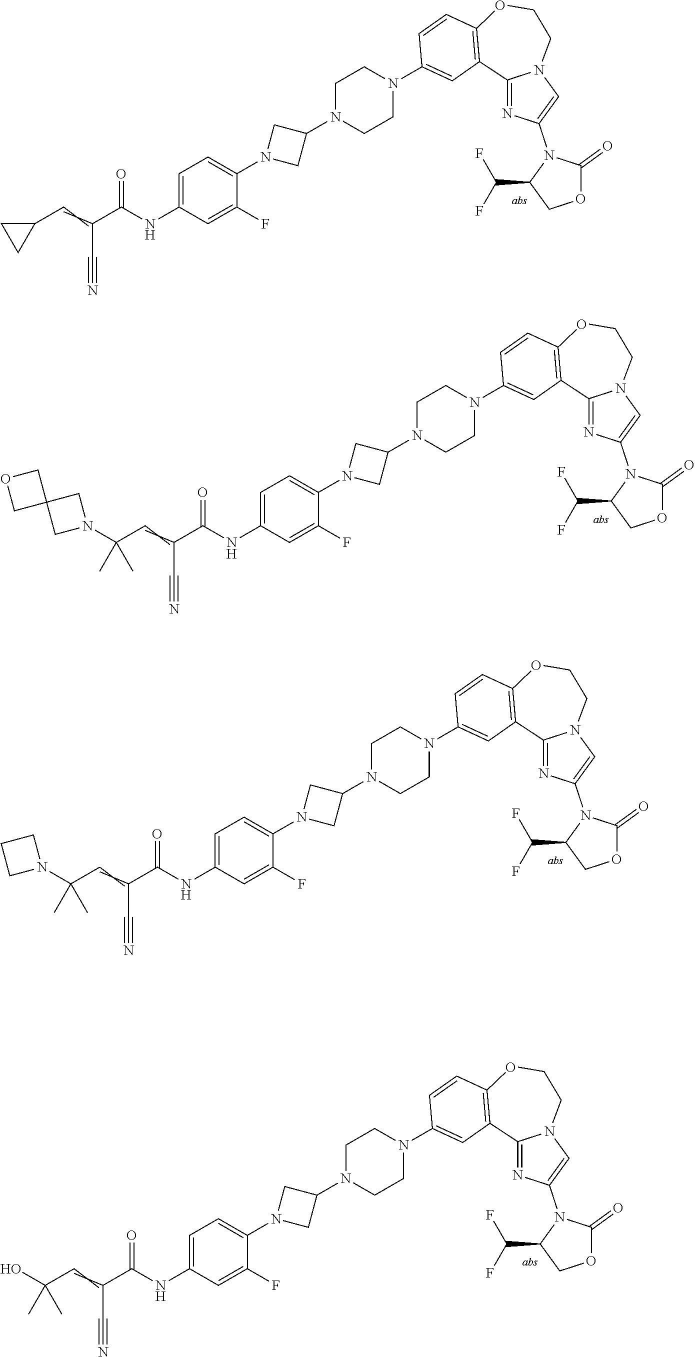 [SMILES: N#CC(=CC1CC1)C(=O)Nc1ccc(N2CC(N3CCN(c4ccc5c(c4)-c4nc(N6C(=O)OC[C@H]6C(F)F)cn4CCO5)CC3)C2)c(F)c1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1)N1CC2(COC2)C1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1)N1CCC1 ; CC(C)(O)C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1]

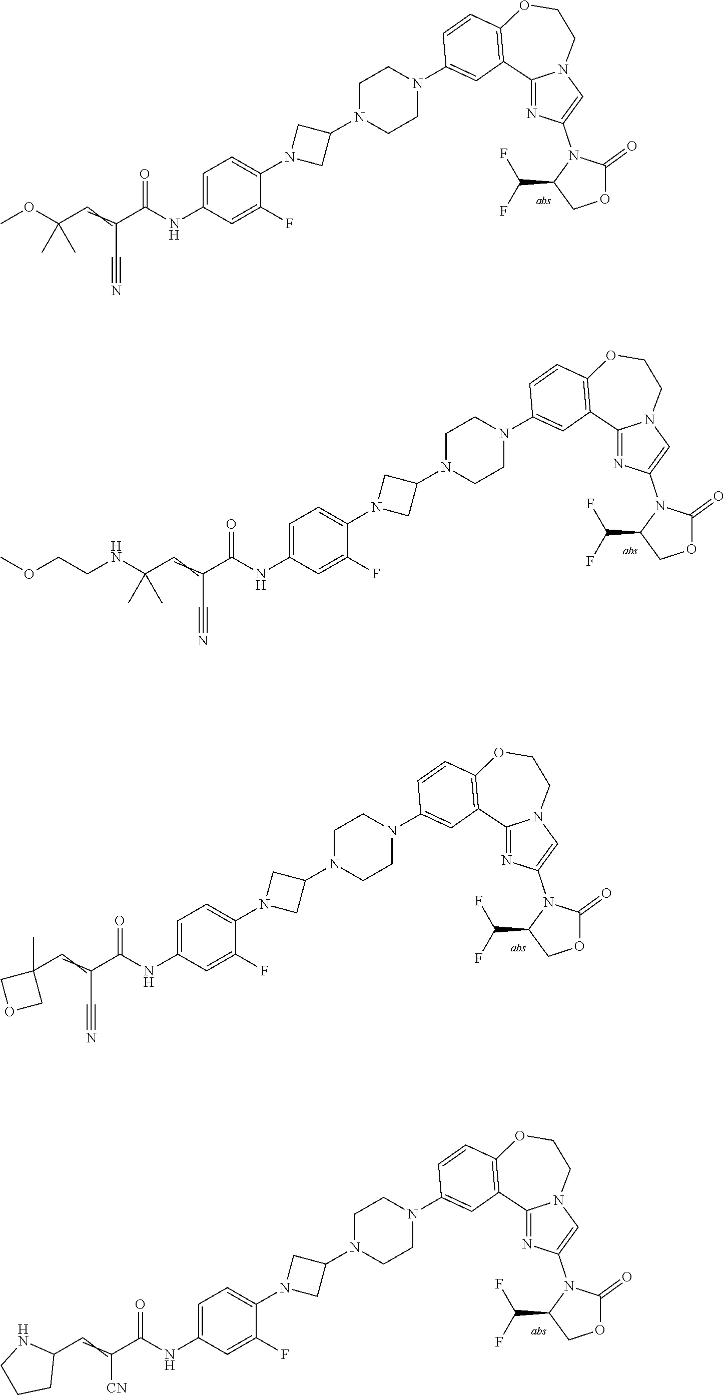 [SMILES: COC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1 ; COCCNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1 ; CC1(C=C(C#N)C(=O)Nc2ccc(N3CC(N4CCN(c5ccc6c(c5)-c5nc(N7C(=O)OC[C@H]7C(F)F)cn5CCO6)CC4)C3)c(F)c2)COC1 ; N#CC(=CC1CCCN1)C(=O)Nc1ccc(N2CC(N3CCN(c4ccc5c(c4)-c4nc(N6C(=O)OC[C@H]6C(F)F)cn4CCO5)CC3)C2)c(F)c1]

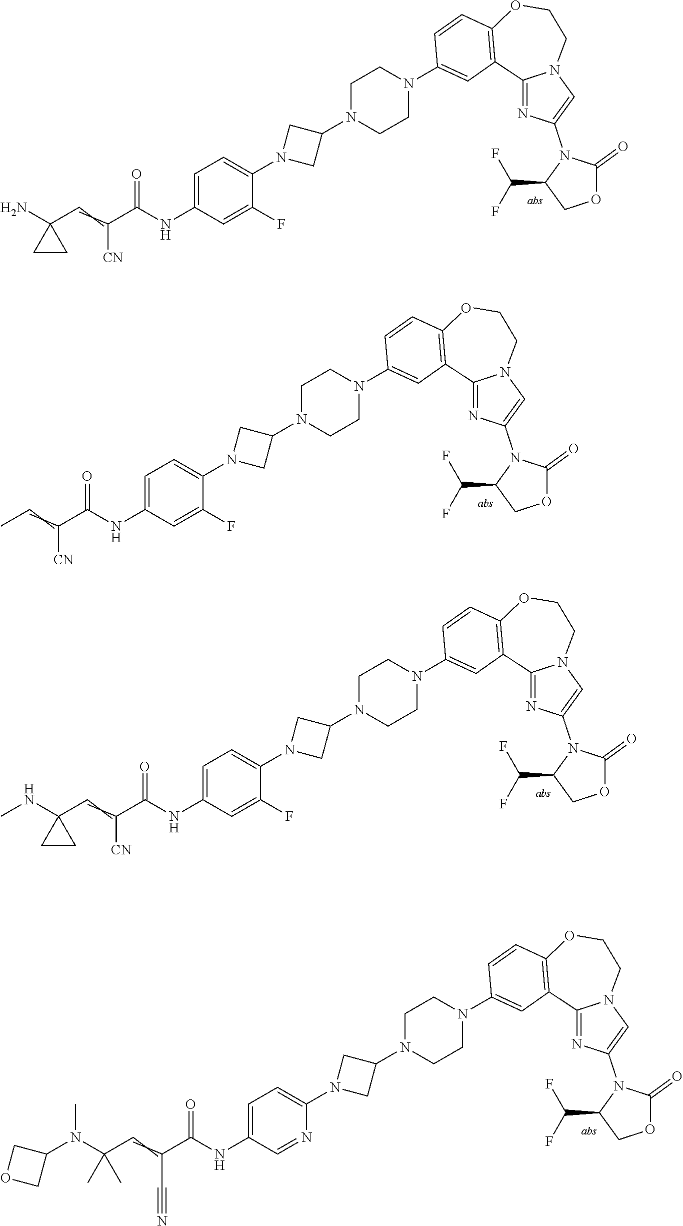 [SMILES: N#CC(=CC1(N)CC1)C(=O)Nc1ccc(N2CC(N3CCN(c4ccc5c(c4)-c4nc(N6C(=O)OC[C@H]6C(F)F)cn4CCO5)CC3)C2)c(F)c1 ; CC=C(C#N)C(=O)Nc1ccc(N2CC(N3CCN(c4ccc5c(c4)-c4nc(N6C(=O)OC[C@H]6C(F)F)cn4CCO5)CC3)C2)c(F)c1 ; CNC1(C=C(C#N)C(=O)Nc2ccc(N3CC(N4CCN(c5ccc6c(c5)-c5nc(N7C(=O)OC[C@H]7C(F)F)cn5CCO6)CC4)C3)c(F)c2)CC1 ; CN(C1COC1)C(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)nc1]

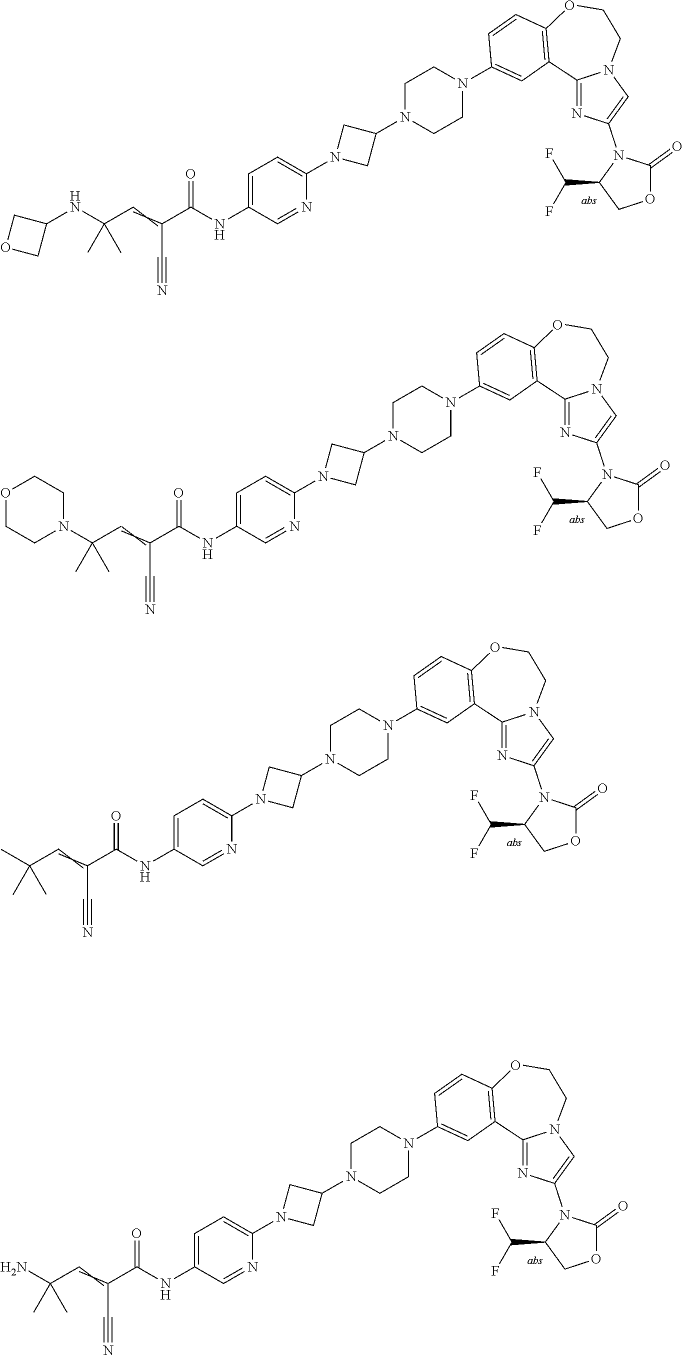 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)nc1)NC1COC1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)nc1)N1CCOCC1 ; CC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)nc1 ; CC(C)(N)C=C(C#N)C(=O)Nc1ccc(N2CC(N3CCN(c4ccc5c(c4)-c4nc(N6C(=O)OC[C@H]6C(F)F)cn4CCO5)CC3)C2)nc1]

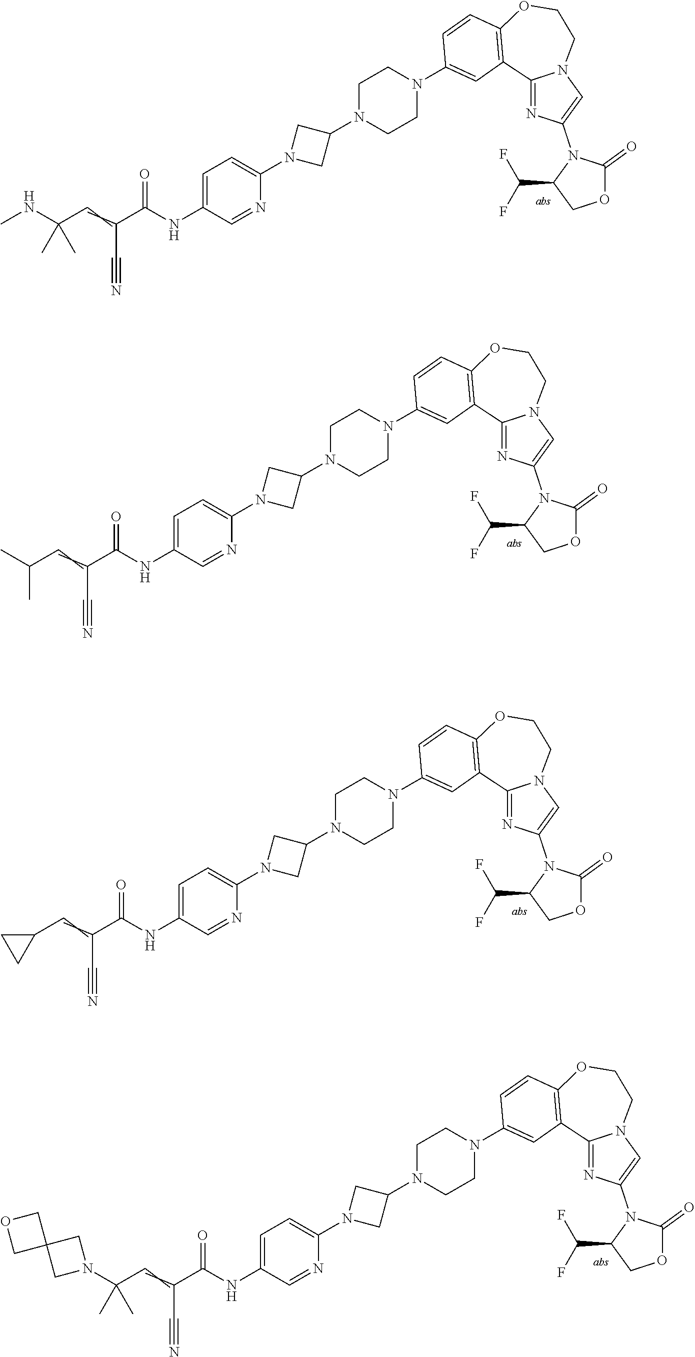 [SMILES: CNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)nc1 ; CC(C)C=C(C#N)C(=O)Nc1ccc(N2CC(N3CCN(c4ccc5c(c4)-c4nc(N6C(=O)OC[C@H]6C(F)F)cn4CCO5)CC3)C2)nc1 ; N#CC(=CC1CC1)C(=O)Nc1ccc(N2CC(N3CCN(c4ccc5c(c4)-c4nc(N6C(=O)OC[C@H]6C(F)F)cn4CCO5)CC3)C2)nc1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)nc1)N1CC2(COC2)C1]

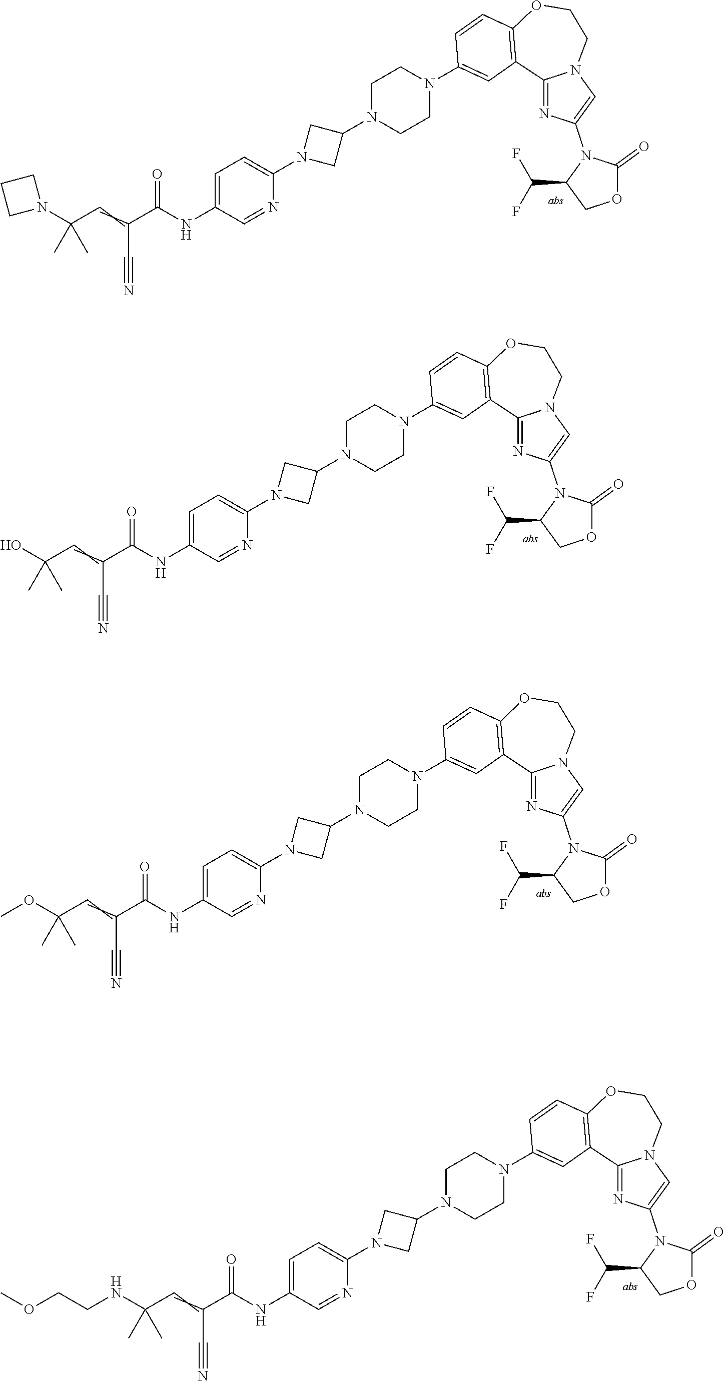 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)nc1)N1CCC1 ; CC(C)(O)C=C(C#N)C(=O)Nc1ccc(N2CC(N3CCN(c4ccc5c(c4)-c4nc(N6C(=O)OC[C@H]6C(F)F)cn4CCO5)CC3)C2)nc1 ; COC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)nc1 ; COCCNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)nc1]

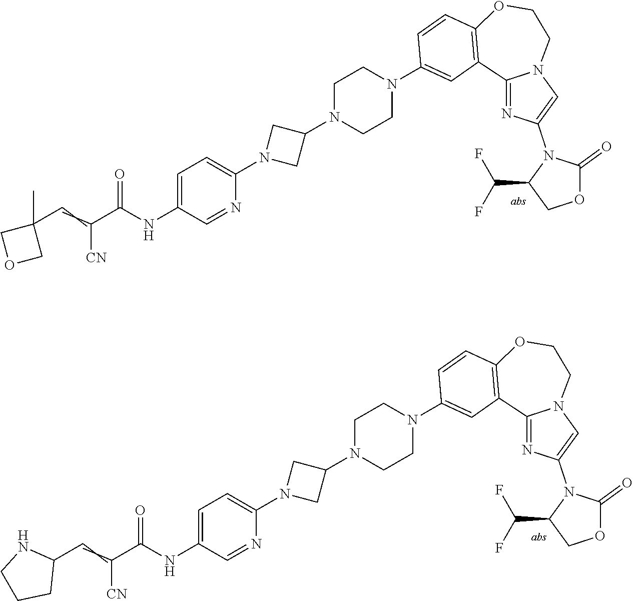 [SMILES: CC1(C=C(C#N)C(=O)Nc2ccc(N3CC(N4CCN(c5ccc6c(c5)-c5nc(N7C(=O)OC[C@H]7C(F)F)cn5CCO6)CC4)C3)nc2)COC1 ; N#CC(=CC1CCCN1)C(=O)Nc1ccc(N2CC(N3CCN(c4ccc5c(c4)-c4nc(N6C(=O)OC[C@H]6C(F)F)cn4CCO5)CC3)C2)nc1]

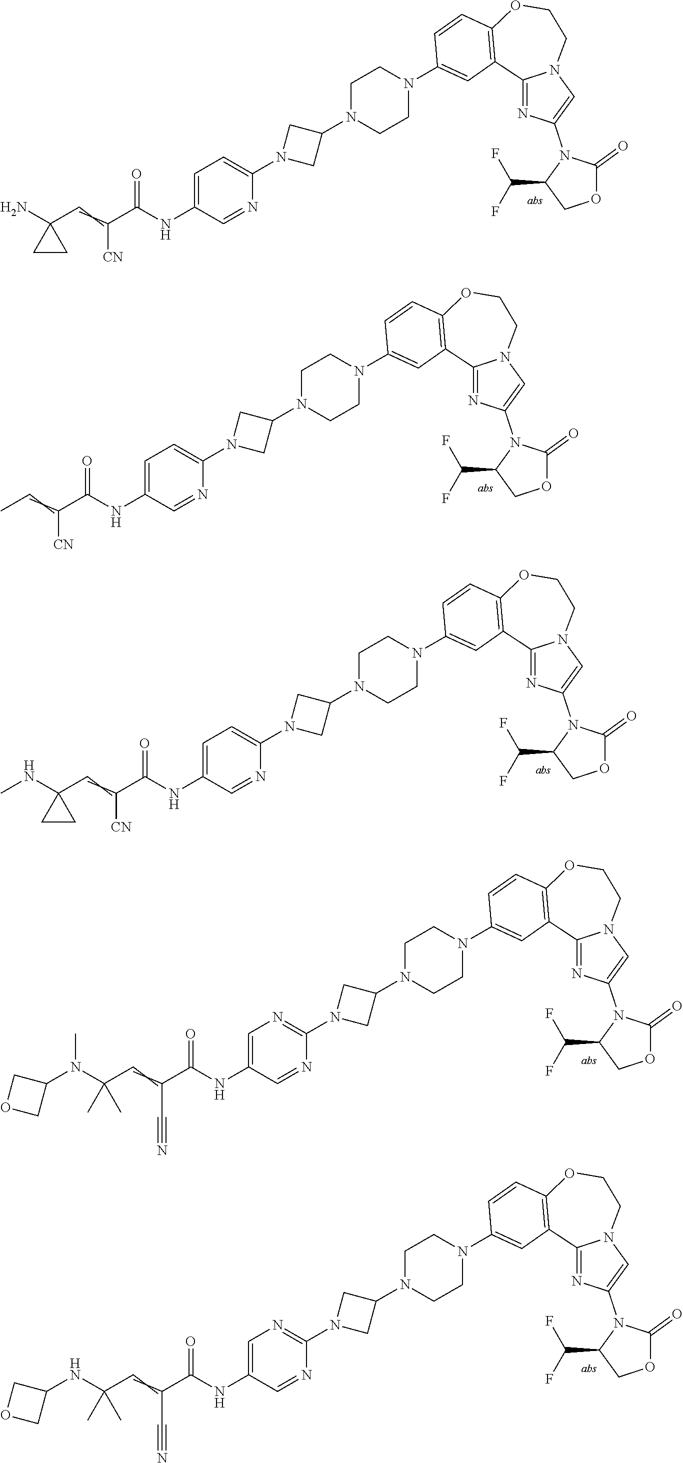 [SMILES: N#CC(=CC1(N)CC1)C(=O)Nc1ccc(N2CC(N3CCN(c4ccc5c(c4)-c4nc(N6C(=O)OC[C@H]6C(F)F)cn4CCO5)CC3)C2)nc1 ; CC=C(C#N)C(=O)Nc1ccc(N2CC(N3CCN(c4ccc5c(c4)-c4nc(N6C(=O)OC[C@H]6C(F)F)cn4CCO5)CC3)C2)nc1 ; CNC1(C=C(C#N)C(=O)Nc2ccc(N3CC(N4CCN(c5ccc6c(c5)-c5nc(N7C(=O)OC[C@H]7C(F)F)cn5CCO6)CC4)C3)nc2)CC1 ; CN(C1COC1)C(C)(C)C=C(C#N)C(=O)Nc1cnc(N2CC(N3CCN(c4ccc5c(c4)-c4nc(N6C(=O)OC[C@H]6C(F)F)cn4CCO5)CC3)C2)nc1 ; CC(C)(C=C(C#N)C(=O)Nc1cnc(N2CC(N3CCN(c4ccc5c(c4)-c4nc(N6C(=O)OC[C@H]6C(F)F)cn4CCO5)CC3)C2)nc1)NC1COC1]

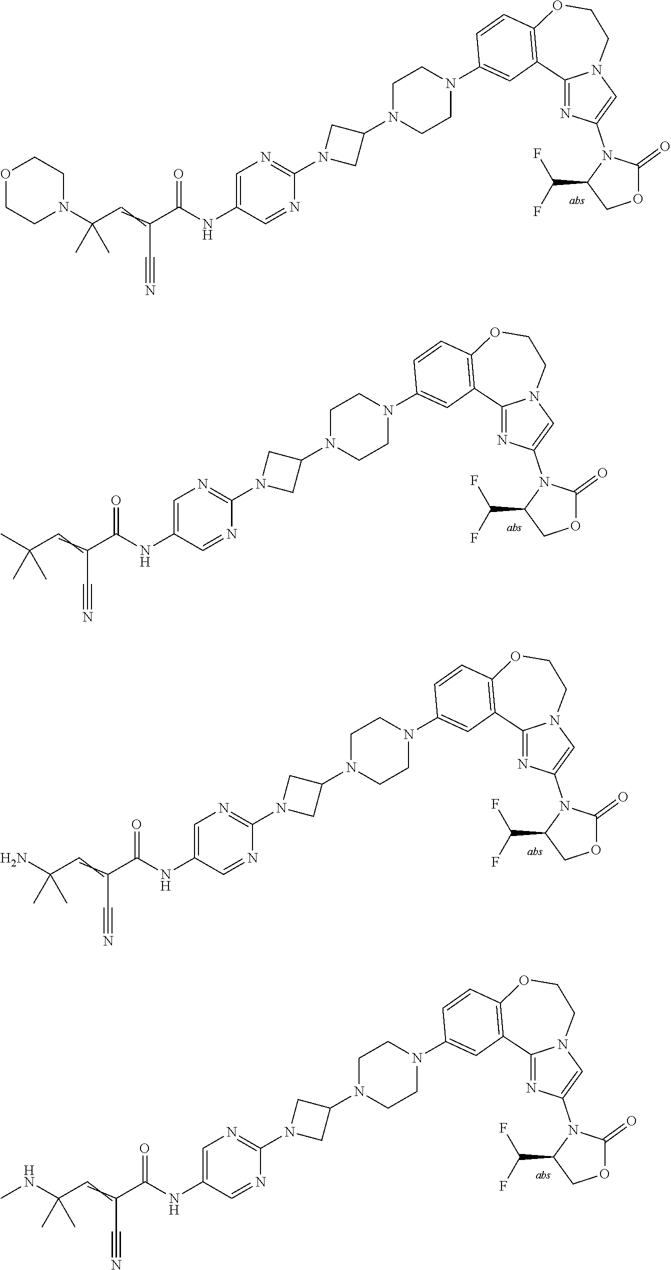 [SMILES: CC(C)(C=C(C#N)C(=O)Nc1cnc(N2CC(N3CCN(c4ccc5c(c4)-c4nc(N6C(=O)OC[C@H]6C(F)F)cn4CCO5)CC3)C2)nc1)N1CCOCC1 ; CC(C)(C)C=C(C#N)C(=O)Nc1cnc(N2CC(N3CCN(c4ccc5c(c4)-c4nc(N6C(=O)OC[C@H]6C(F)F)cn4CCO5)CC3)C2)nc1 ; CC(C)(N)C=C(C#N)C(=O)Nc1cnc(N2CC(N3CCN(c4ccc5c(c4)-c4nc(N6C(=O)OC[C@H]6C(F)F)cn4CCO5)CC3)C2)nc1 ; CNC(C)(C)C=C(C#N)C(=O)Nc1cnc(N2CC(N3CCN(c4ccc5c(c4)-c4nc(N6C(=O)OC[C@H]6C(F)F)cn4CCO5)CC3)C2)nc1]

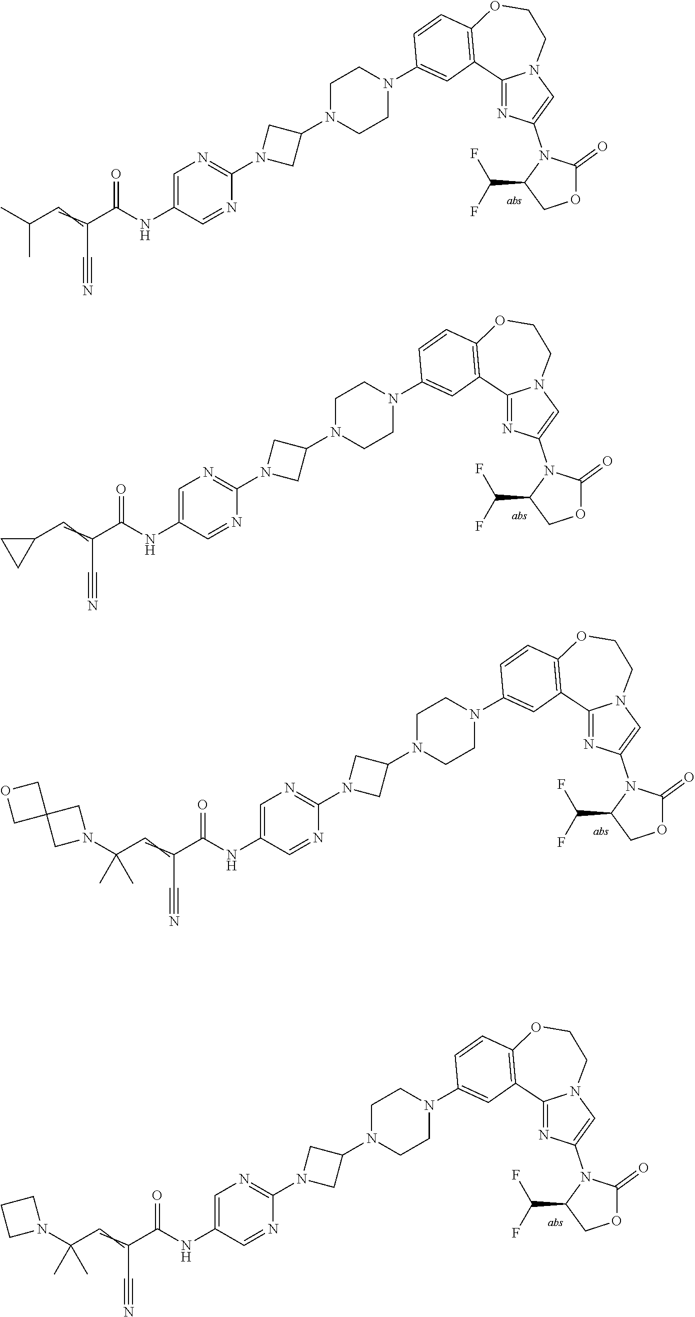 [SMILES: CC(C)C=C(C#N)C(=O)Nc1cnc(N2CC(N3CCN(c4ccc5c(c4)-c4nc(N6C(=O)OC[C@H]6C(F)F)cn4CCO5)CC3)C2)nc1 ; N#CC(=CC1CC1)C(=O)Nc1cnc(N2CC(N3CCN(c4ccc5c(c4)-c4nc(N6C(=O)OC[C@H]6C(F)F)cn4CCO5)CC3)C2)nc1 ; CC(C)(C=C(C#N)C(=O)Nc1cnc(N2CC(N3CCN(c4ccc5c(c4)-c4nc(N6C(=O)OC[C@H]6C(F)F)cn4CCO5)CC3)C2)nc1)N1CC2(COC2)C1 ; CC(C)(C=C(C#N)C(=O)Nc1cnc(N2CC(N3CCN(c4ccc5c(c4)-c4nc(N6C(=O)OC[C@H]6C(F)F)cn4CCO5)CC3)C2)nc1)N1CCC1]

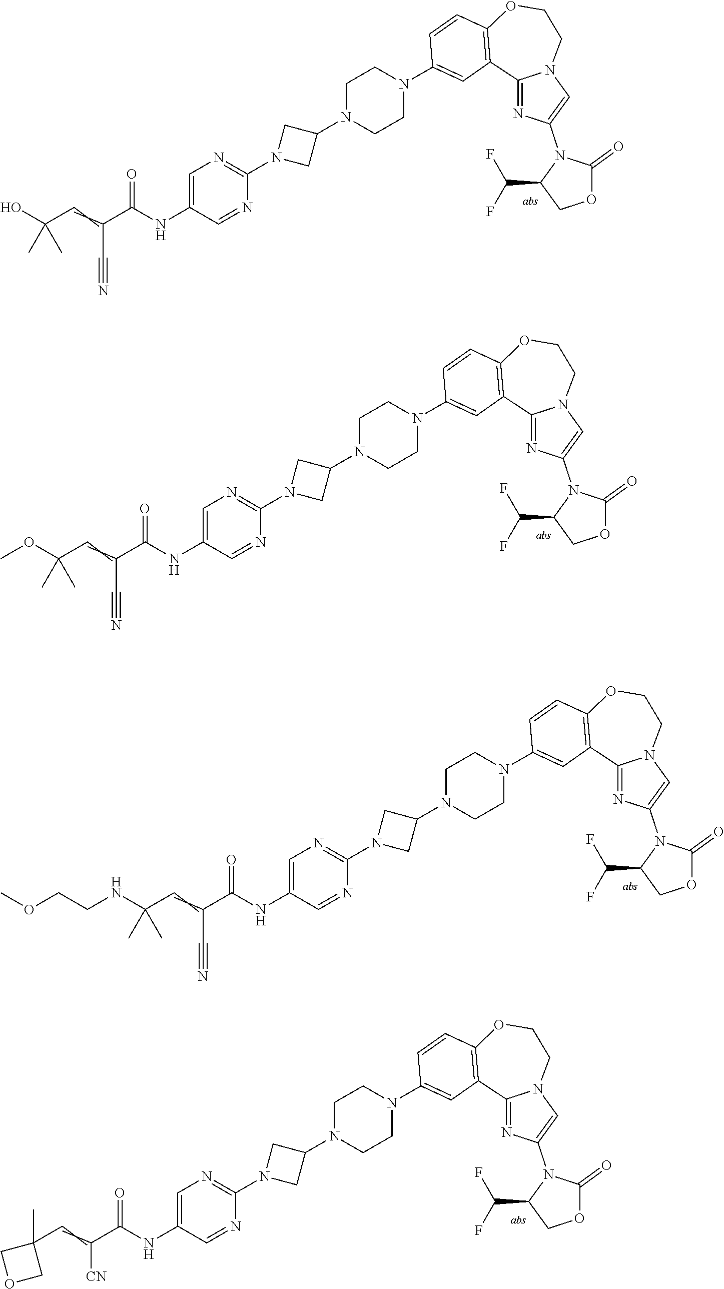 [SMILES: CC(C)(O)C=C(C#N)C(=O)Nc1cnc(N2CC(N3CCN(c4ccc5c(c4)-c4nc(N6C(=O)OC[C@H]6C(F)F)cn4CCO5)CC3)C2)nc1 ; COC(C)(C)C=C(C#N)C(=O)Nc1cnc(N2CC(N3CCN(c4ccc5c(c4)-c4nc(N6C(=O)OC[C@H]6C(F)F)cn4CCO5)CC3)C2)nc1 ; COCCNC(C)(C)C=C(C#N)C(=O)Nc1cnc(N2CC(N3CCN(c4ccc5c(c4)-c4nc(N6C(=O)OC[C@H]6C(F)F)cn4CCO5)CC3)C2)nc1 ; CC1(C=C(C#N)C(=O)Nc2cnc(N3CC(N4CCN(c5ccc6c(c5)-c5nc(N7C(=O)OC[C@H]7C(F)F)cn5CCO6)CC4)C3)nc2)COC1]

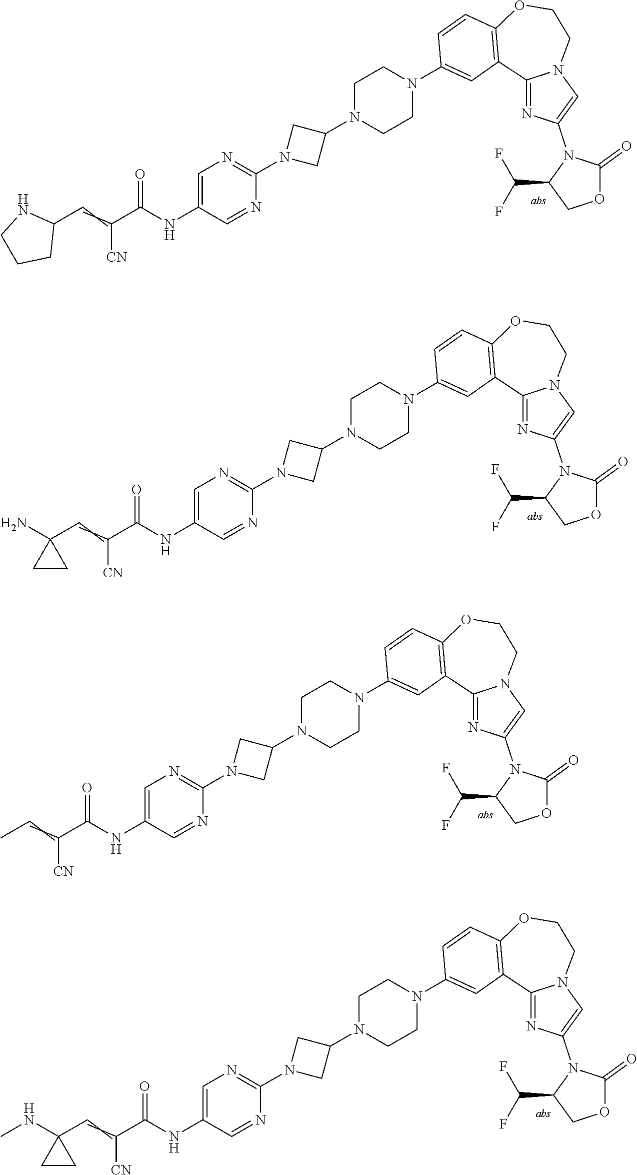 [SMILES: N#CC(=CC1CCCN1)C(=O)Nc1cnc(N2CC(N3CCN(c4ccc5c(c4)-c4nc(N6C(=O)OC[C@H]6C(F)F)cn4CCO5)CC3)C2)nc1 ; N#CC(=CC1(N)CC1)C(=O)Nc1cnc(N2CC(N3CCN(c4ccc5c(c4)-c4nc(N6C(=O)OC[C@H]6C(F)F)cn4CCO5)CC3)C2)nc1 ; CC=C(C#N)C(=O)Nc1cnc(N2CC(N3CCN(c4ccc5c(c4)-c4nc(N6C(=O)OC[C@H]6C(F)F)cn4CCO5)CC3)C2)nc1 ; CNC1(C=C(C#N)C(=O)Nc2cnc(N3CC(N4CCN(c5ccc6c(c5)-c5nc(N7C(=O)OC[C@H]7C(F)F)cn5CCO6)CC4)C3)nc2)CC1]

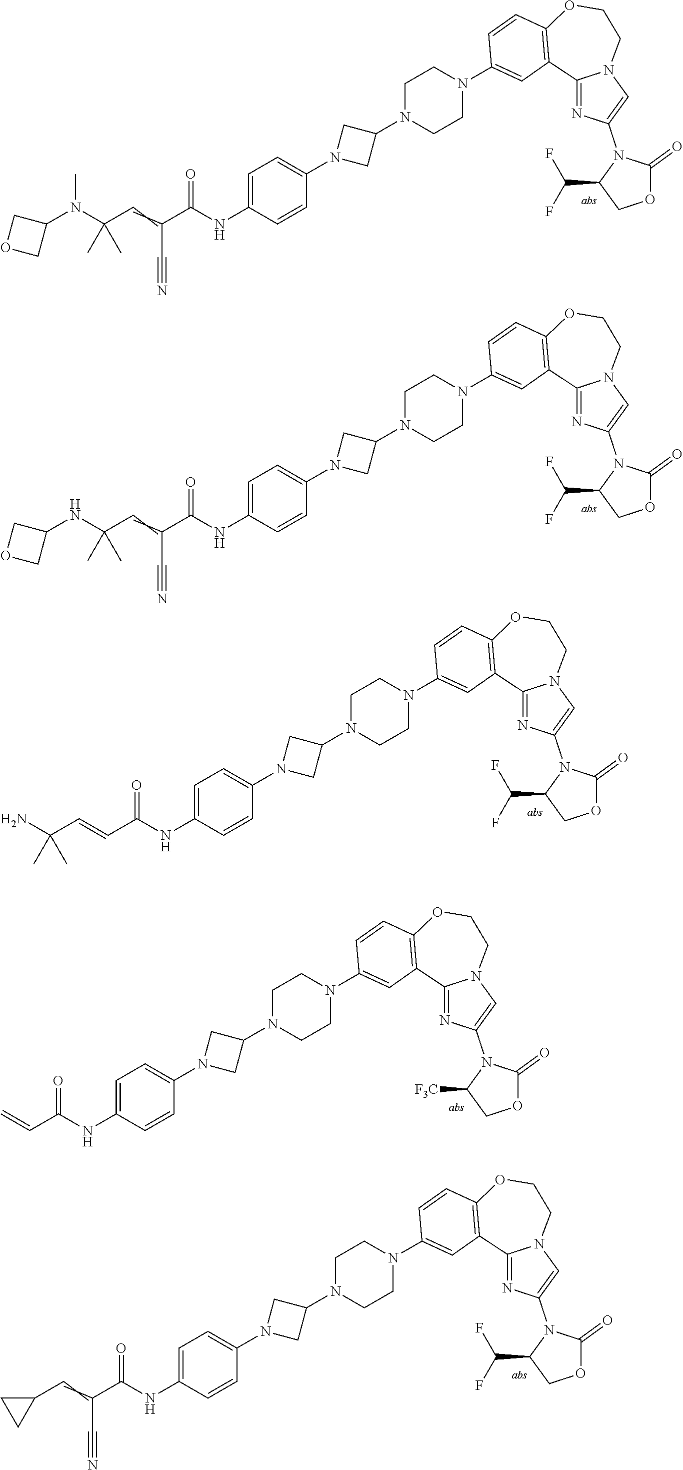 [SMILES: CN(C1COC1)C(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)NC1COC1 ; CC(C)(N)/C=C/C(=O)Nc1ccc(N2CC(N3CCN(c4ccc5c(c4)-c4nc(N6C(=O)OC[C@H]6C(F)F)cn4CCO5)CC3)C2)cc1 ; C=CC(=O)Nc1ccc(N2CC(N3CCN(c4ccc5c(c4)-c4nc(N6C(=O)OC[C@H]6C(F)(F)F)cn4CCO5)CC3)C2)cc1 ; N#CC(=CC1CC1)C(=O)Nc1ccc(N2CC(N3CCN(c4ccc5c(c4)-c4nc(N6C(=O)OC[C@H]6C(F)F)cn4CCO5)CC3)C2)cc1]

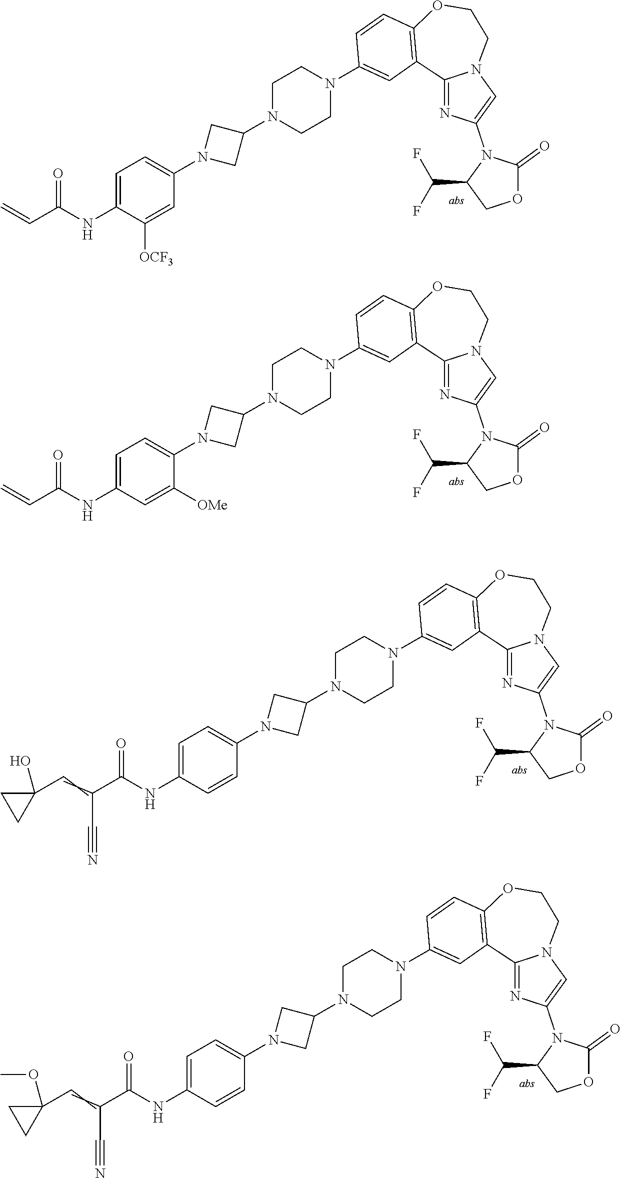 [SMILES: C=CC(=O)Nc1ccc(N2CC(N3CCN(c4ccc5c(c4)-c4nc(N6C(=O)OC[C@H]6C(F)F)cn4CCO5)CC3)C2)cc1OC(F)(F)F ; C=CC(=O)Nc1ccc(N2CC(N3CCN(c4ccc5c(c4)-c4nc(N6C(=O)OC[C@H]6C(F)F)cn4CCO5)CC3)C2)c(OC)c1 ; N#CC(=CC1(O)CC1)C(=O)Nc1ccc(N2CC(N3CCN(c4ccc5c(c4)-c4nc(N6C(=O)OC[C@H]6C(F)F)cn4CCO5)CC3)C2)cc1 ; COC1(C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CC1]

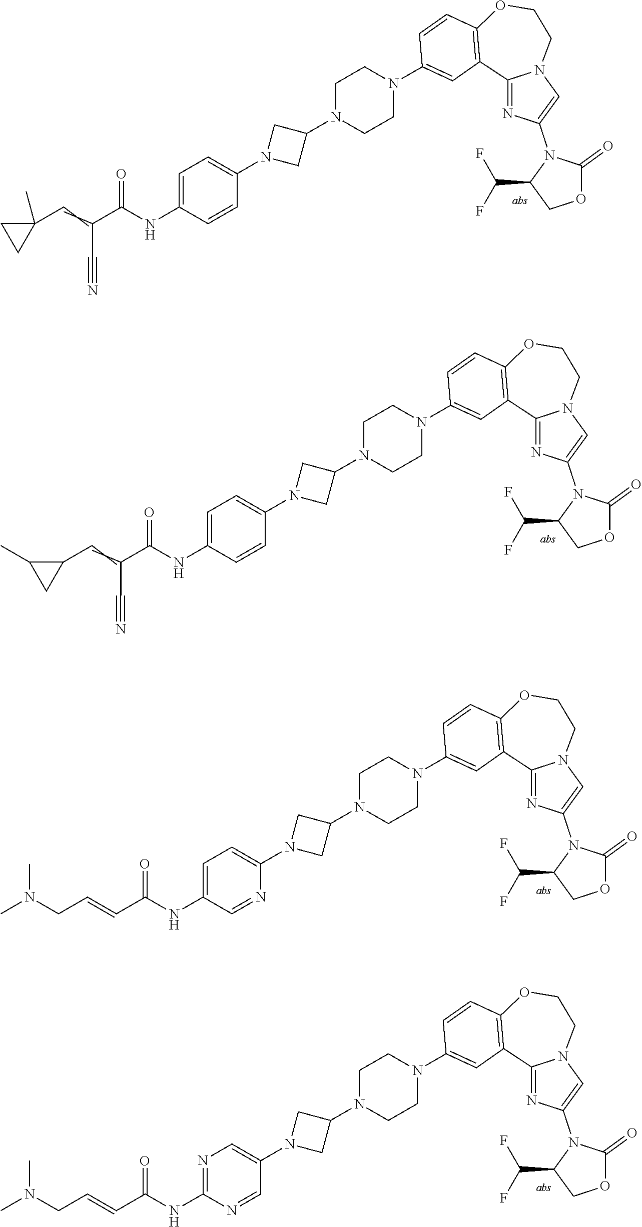 [SMILES: CC1(C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CC1 ; CC1CC1C=C(C#N)C(=O)Nc1ccc(N2CC(N3CCN(c4ccc5c(c4)-c4nc(N6C(=O)OC[C@H]6C(F)F)cn4CCO5)CC3)C2)cc1 ; CN(C)C/C=C/C(=O)Nc1ccc(N2CC(N3CCN(c4ccc5c(c4)-c4nc(N6C(=O)OC[C@H]6C(F)F)cn4CCO5)CC3)C2)nc1 ; CN(C)C/C=C/C(=O)Nc1ncc(N2CC(N3CCN(c4ccc5c(c4)-c4nc(N6C(=O)OC[C@H]6C(F)F)cn4CCO5)CC3)C2)cn1]

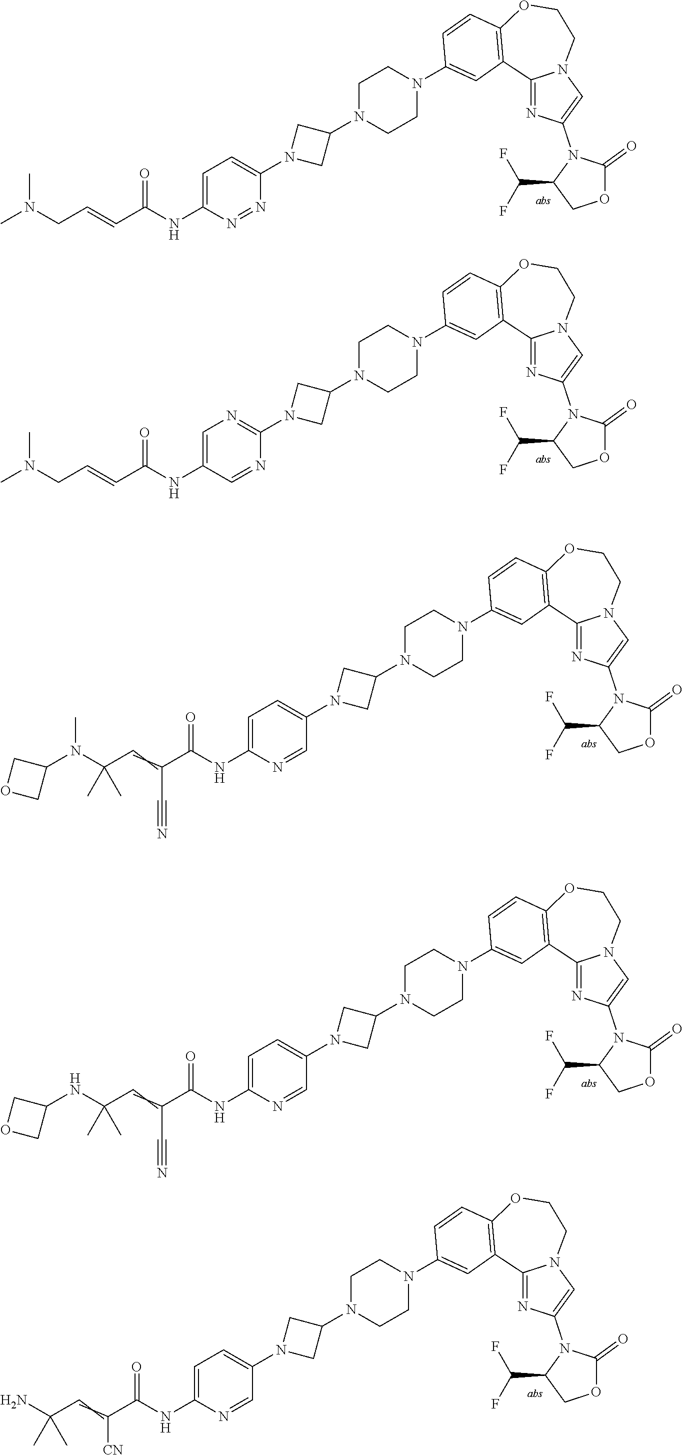 [SMILES: CN(C)C/C=C/C(=O)Nc1ccc(N2CC(N3CCN(c4ccc5c(c4)-c4nc(N6C(=O)OC[C@H]6C(F)F)cn4CCO5)CC3)C2)nn1 ; CN(C)C/C=C/C(=O)Nc1cnc(N2CC(N3CCN(c4ccc5c(c4)-c4nc(N6C(=O)OC[C@H]6C(F)F)cn4CCO5)CC3)C2)nc1 ; CN(C1COC1)C(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cn1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cn1)NC1COC1 ; CC(C)(N)C=C(C#N)C(=O)Nc1ccc(N2CC(N3CCN(c4ccc5c(c4)-c4nc(N6C(=O)OC[C@H]6C(F)F)cn4CCO5)CC3)C2)cn1]

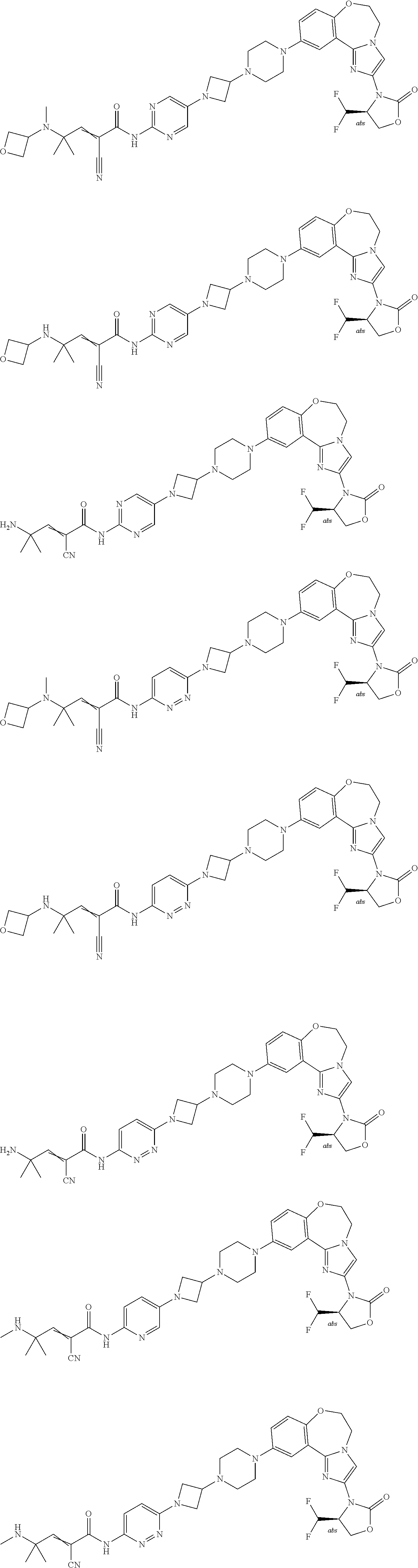

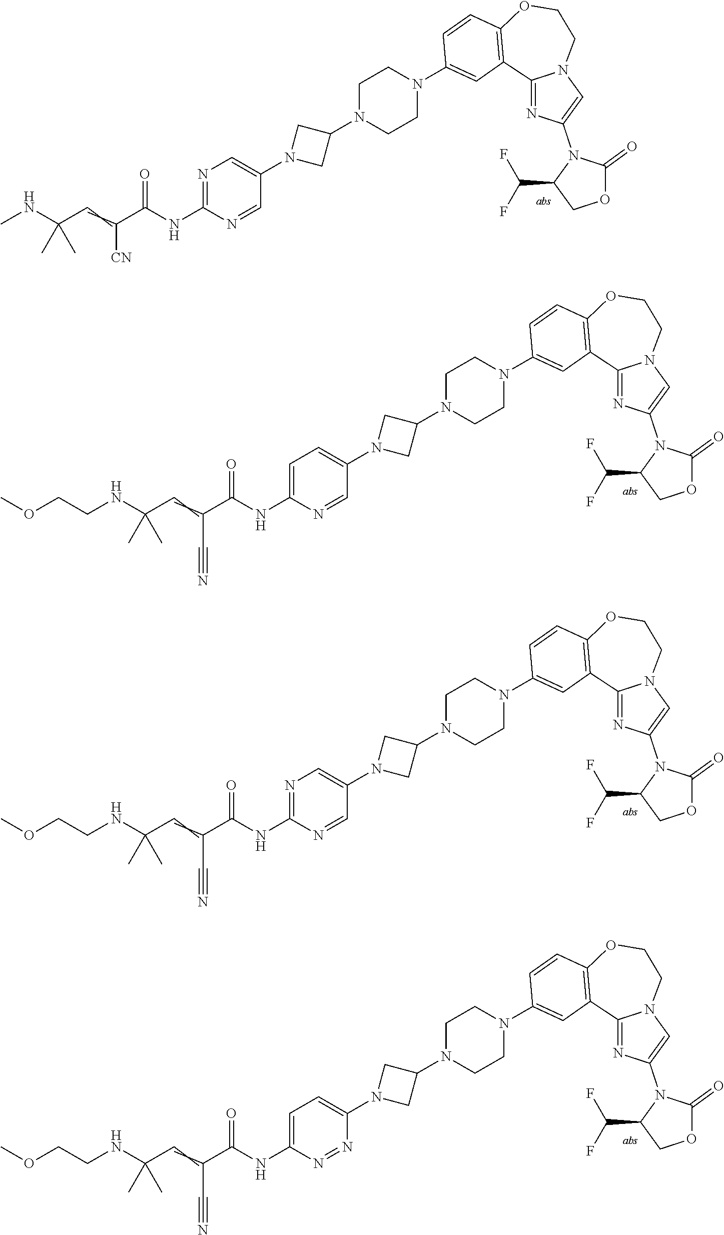 [SMILES: CNC(C)(C)C=C(C#N)C(=O)Nc1ncc(N2CC(N3CCN(c4ccc5c(c4)-c4nc(N6C(=O)OC[C@H]6C(F)F)cn4CCO5)CC3)C2)cn1 ; COCCNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cn1 ; COCCNC(C)(C)C=C(C#N)C(=O)Nc1ncc(N2CC(N3CCN(c4ccc5c(c4)-c4nc(N6C(=O)OC[C@H]6C(F)F)cn4CCO5)CC3)C2)cn1 ; COCCNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)nn1]

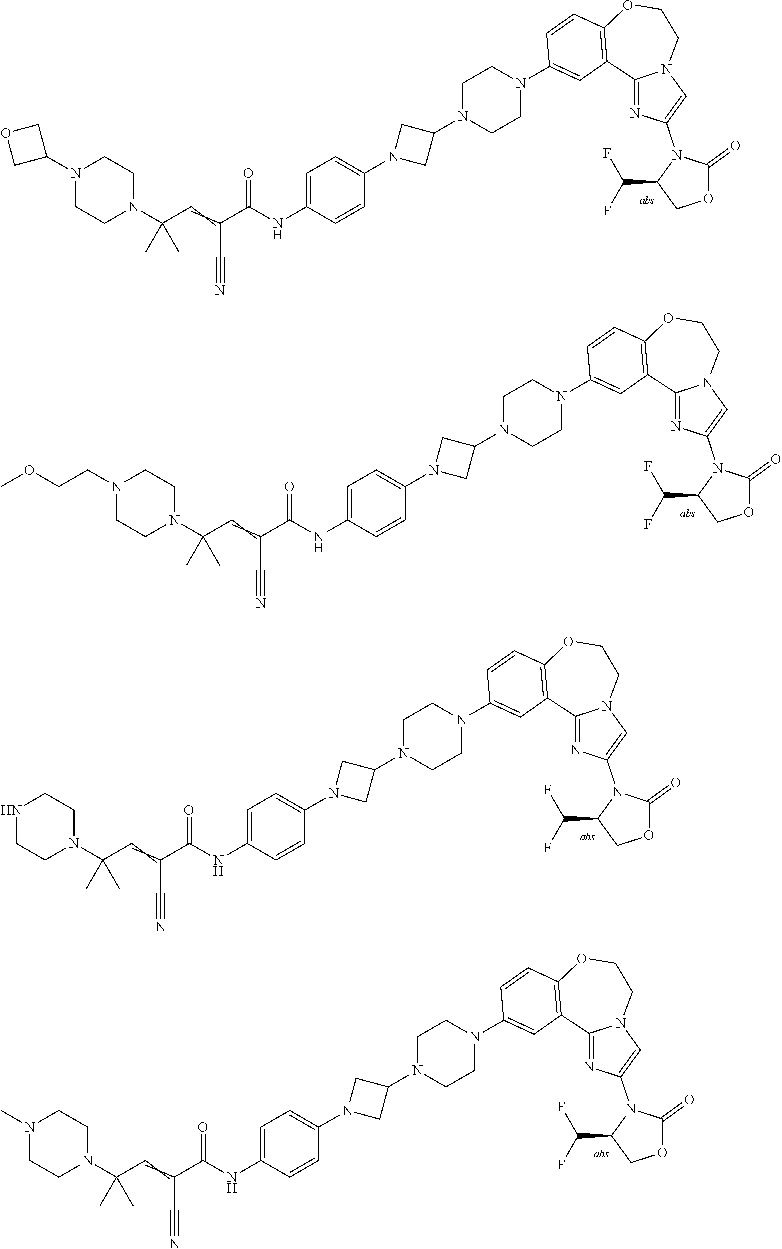 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)N1CCN(C2COC2)CC1 ; COCCN1CCN(C(C)(C)C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CC1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)N1CCNCC1 ; CN1CCN(C(C)(C)C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CC1]

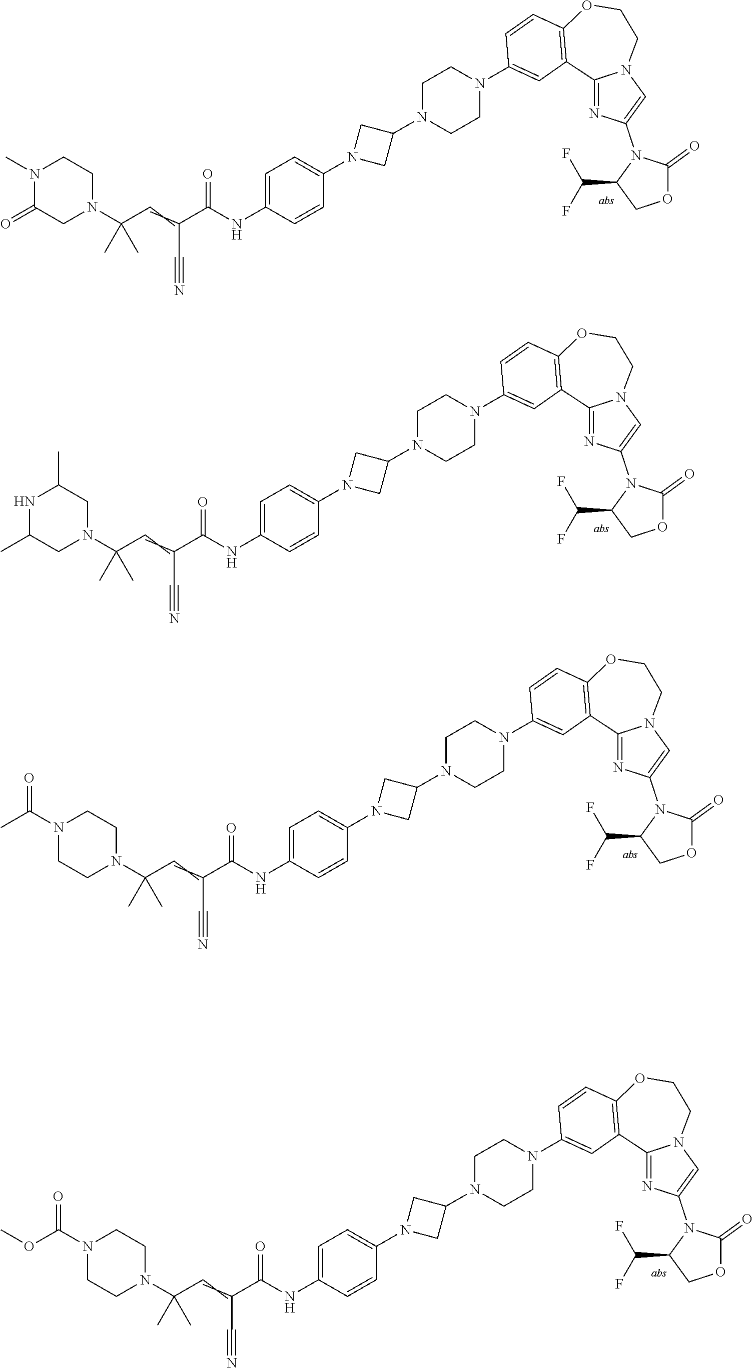 [SMILES: CN1CCN(C(C)(C)C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CC1=O ; CC1CN(C(C)(C)C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CC(C)N1 ; CC(=O)N1CCN(C(C)(C)C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CC1 ; COC(=O)N1CCN(C(C)(C)C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CC1]

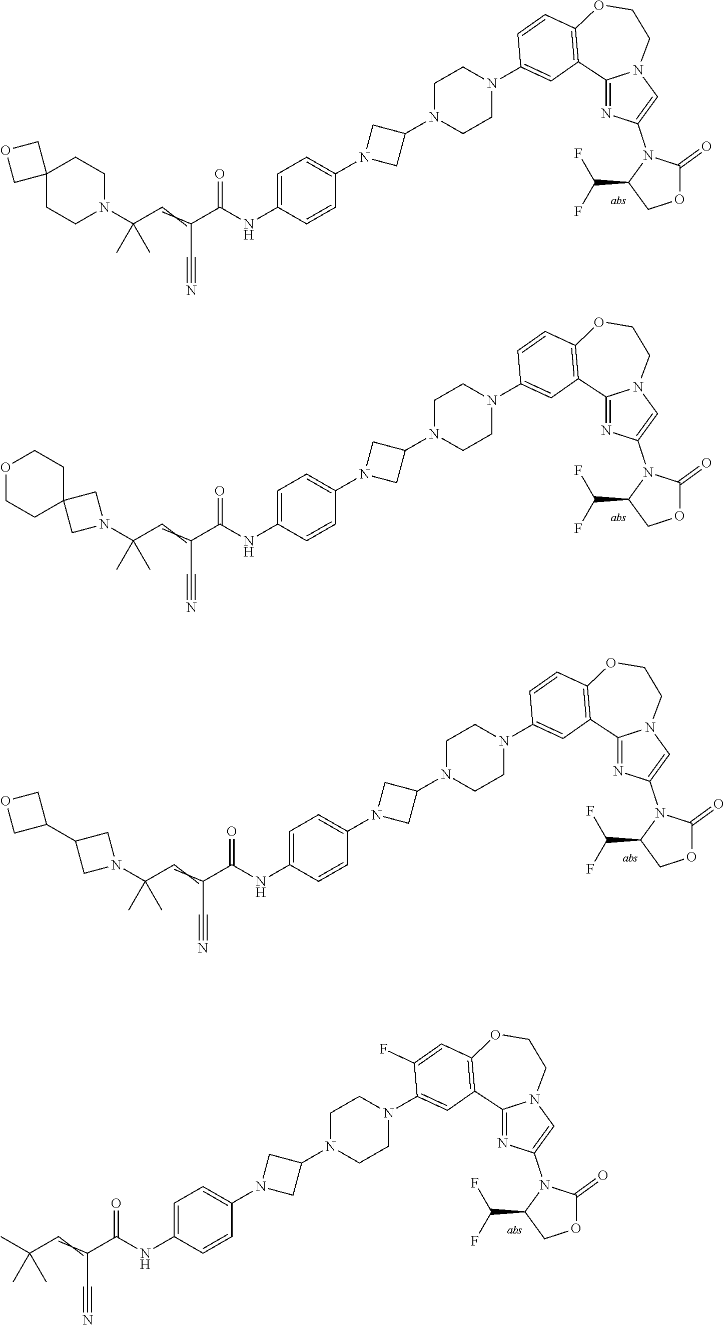 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)N1CCC2(CC1)COC2 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)N1CC2(CCOCC2)C1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)N1CC(C2COC2)C1 ; CC(C)(C)C=C(C#N)C(=O)Nc1ccc(N2CC(N3CCN(c4cc5c(cc4F)OCCn4cc(N6C(=O)OC[C@H]6C(F)F)nc4-5)CC3)C2)cc1]

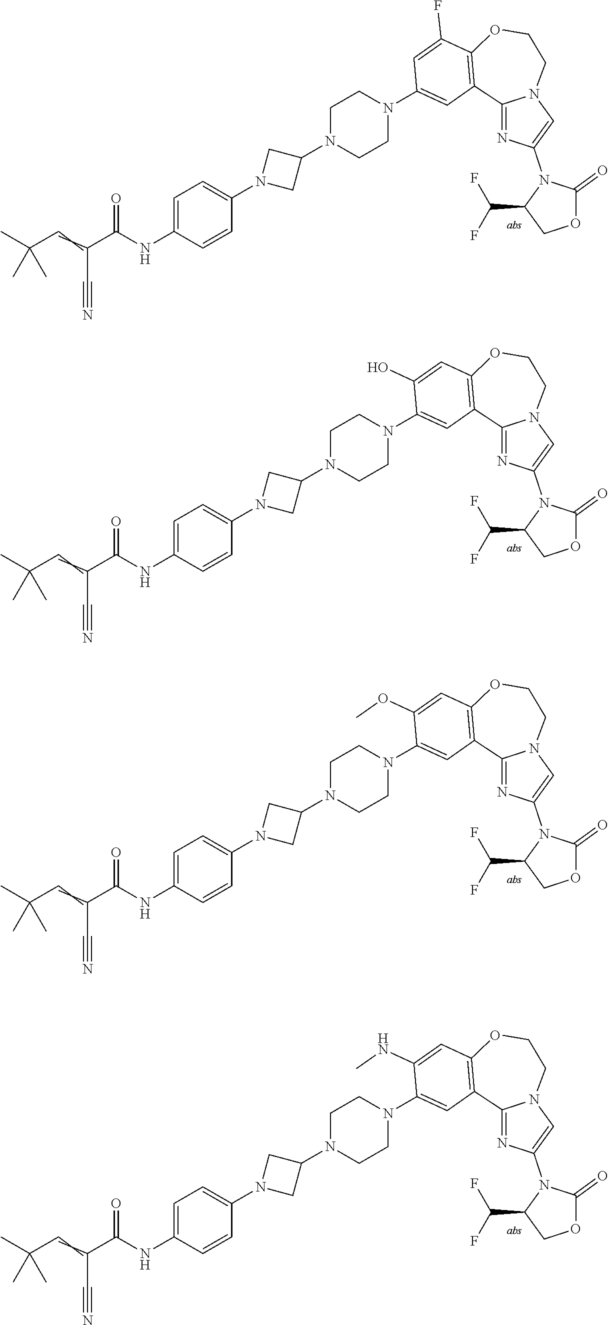 [SMILES: CC(C)(C)C=C(C#N)C(=O)Nc1ccc(N2CC(N3CCN(c4cc(F)c5c(c4)-c4nc(N6C(=O)OC[C@H]6C(F)F)cn4CCO5)CC3)C2)cc1 ; CC(C)(C)C=C(C#N)C(=O)Nc1ccc(N2CC(N3CCN(c4cc5c(cc4O)OCCn4cc(N6C(=O)OC[C@H]6C(F)F)nc4-5)CC3)C2)cc1 ; COc1cc2c(cc1N1CCN(C3CN(c4ccc(NC(=O)C(C#N)=CC(C)(C)C)cc4)C3)CC1)-c1nc(N3C(=O)OC[C@H]3C(F)F)cn1CCO2 ; CNc1cc2c(cc1N1CCN(C3CN(c4ccc(NC(=O)C(C#N)=CC(C)(C)C)cc4)C3)CC1)-c1nc(N3C(=O)OC[C@H]3C(F)F)cn1CCO2]

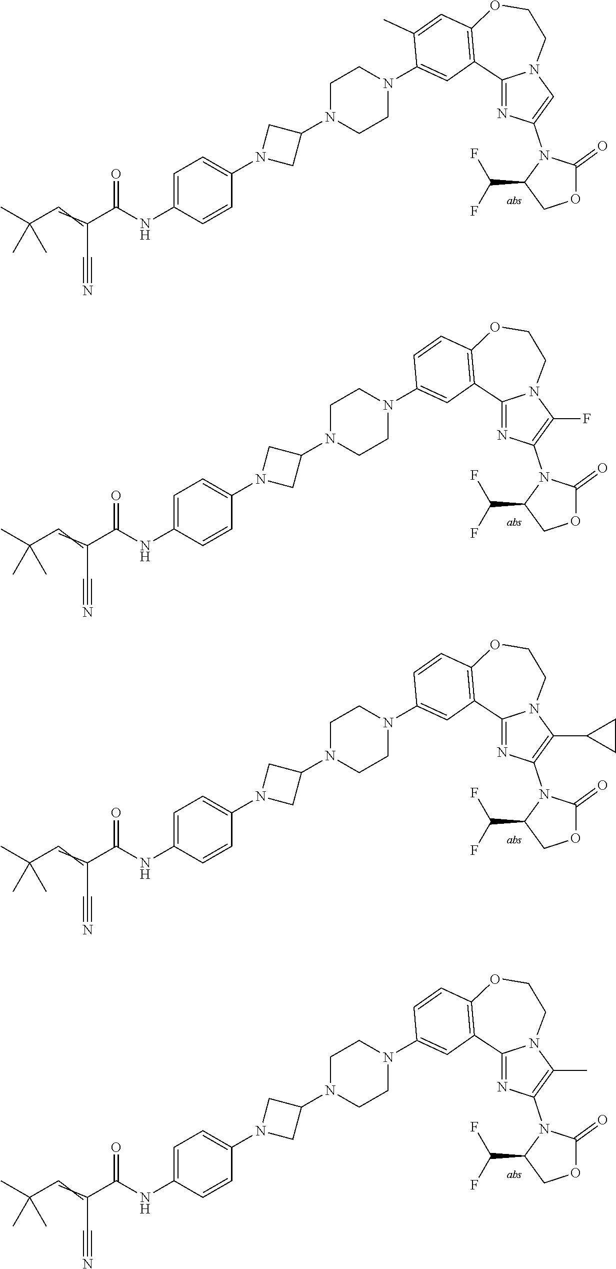 [SMILES: Cc1cc2c(cc1N1CCN(C3CN(c4ccc(NC(=O)C(C#N)=CC(C)(C)C)cc4)C3)CC1)-c1nc(N3C(=O)OC[C@H]3C(F)F)cn1CCO2 ; CC(C)(C)C=C(C#N)C(=O)Nc1ccc(N2CC(N3CCN(c4ccc5c(c4)-c4nc(N6C(=O)OC[C@H]6C(F)F)c(F)n4CCO5)CC3)C2)cc1 ; CC(C)(C)C=C(C#N)C(=O)Nc1ccc(N2CC(N3CCN(c4ccc5c(c4)-c4nc(N6C(=O)OC[C@H]6C(F)F)c(C6CC6)n4CCO5)CC3)C2)cc1 ; Cc1c(N2C(=O)OC[C@H]2C(F)F)nc2n1CCOc1ccc(N3CCN(C4CN(c5ccc(NC(=O)C(C#N)=CC(C)(C)C)cc5)C4)CC3)cc1-2]

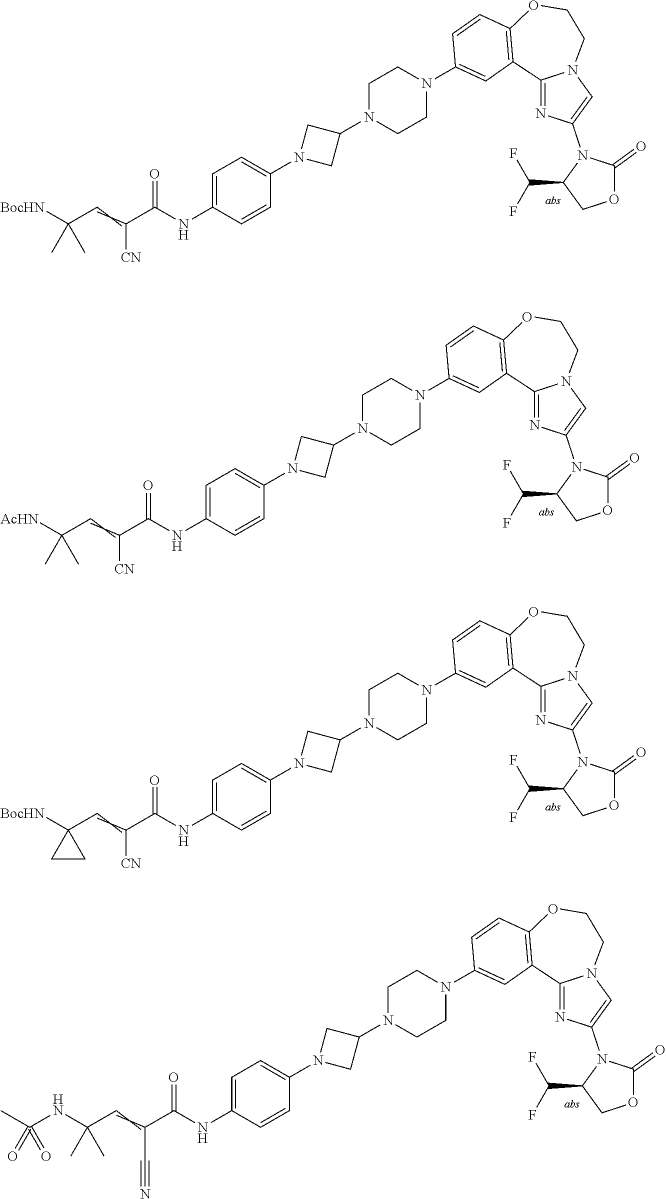

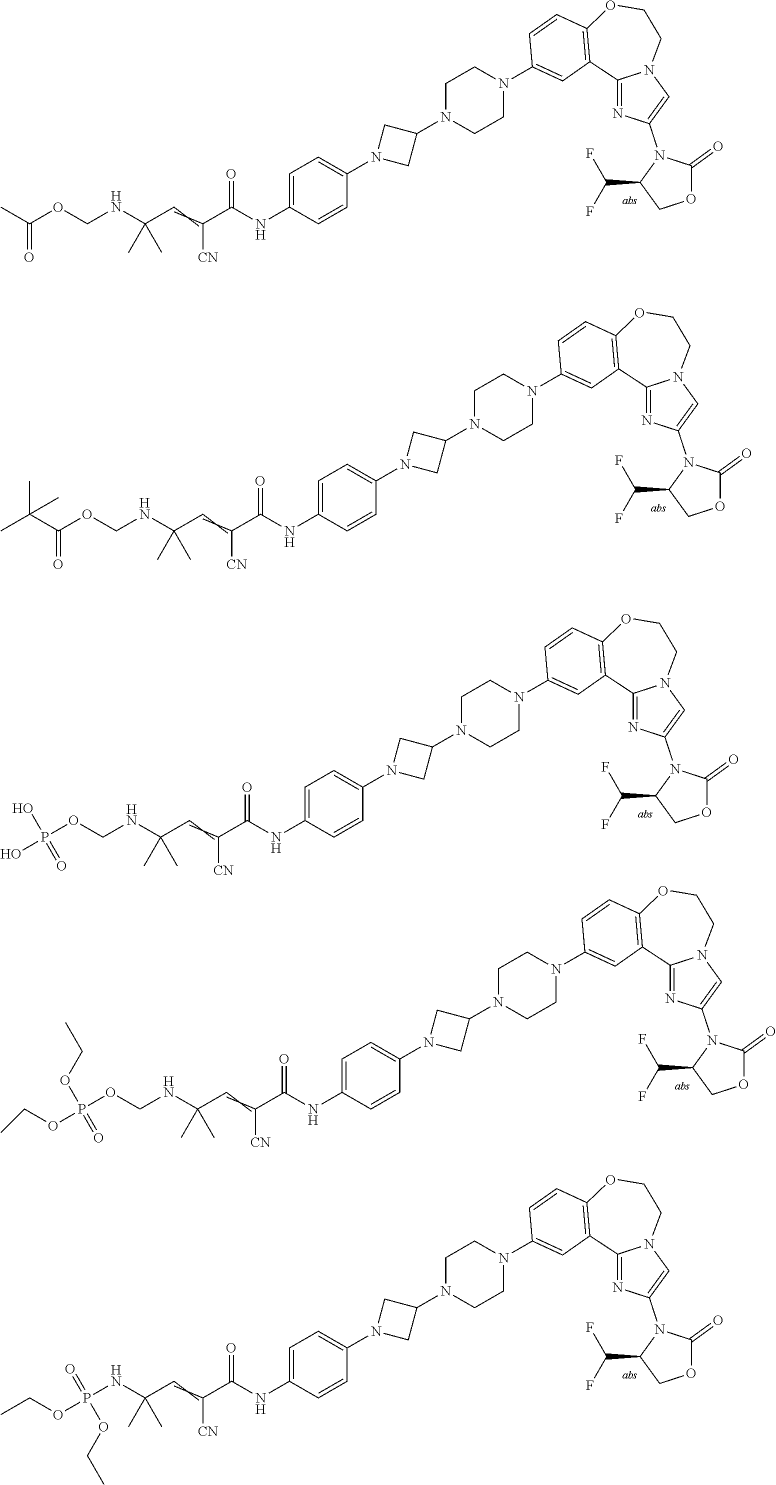

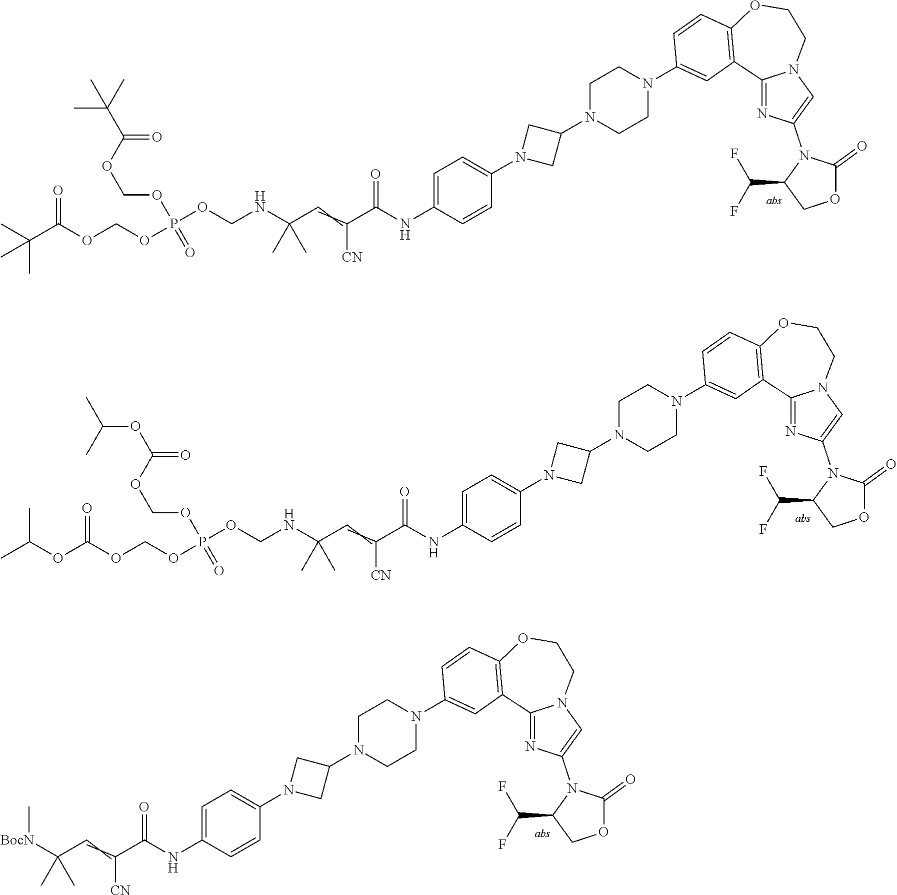 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)NCOP(=O)(OCOC(=O)C(C)(C)C)OCOC(=O)C(C)(C)C ; CC(C)OC(=O)OCOP(=O)(OCNC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1)OCOC(=O)OC(C)C ; CN(C(=O)OC(C)(C)C)C(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1]

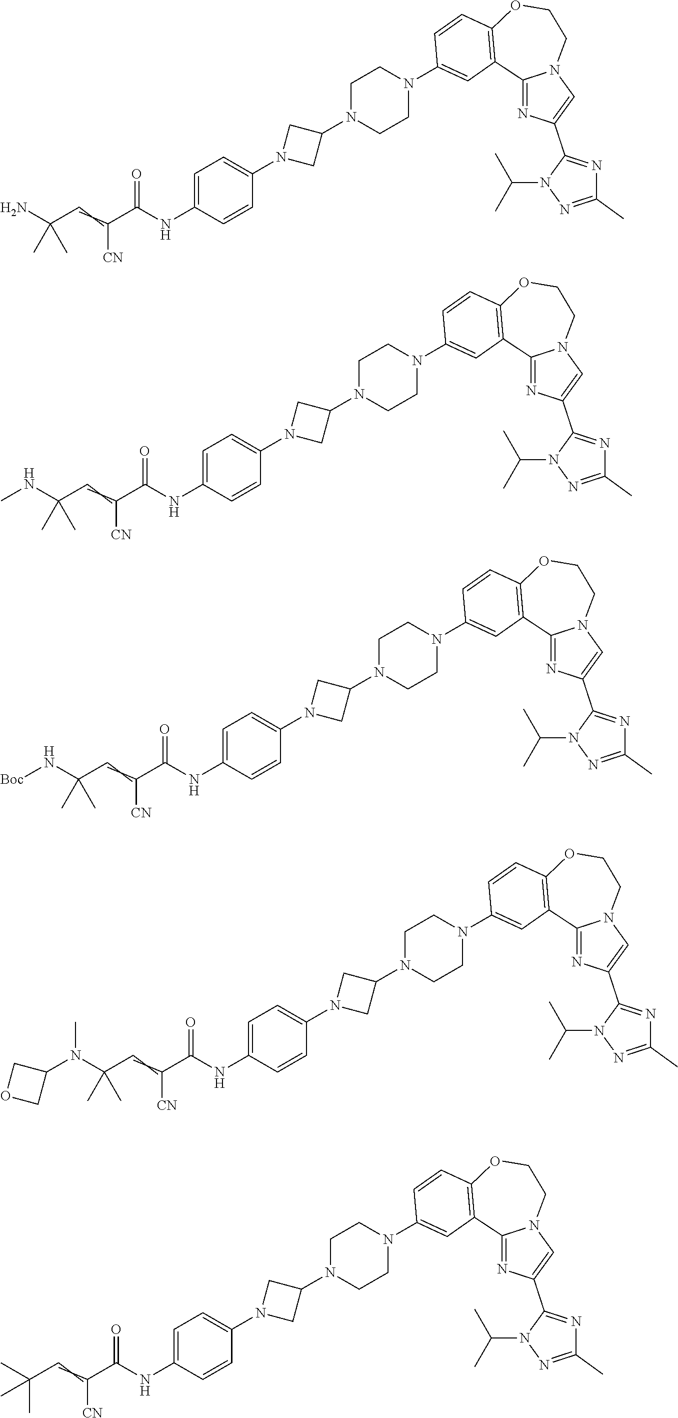

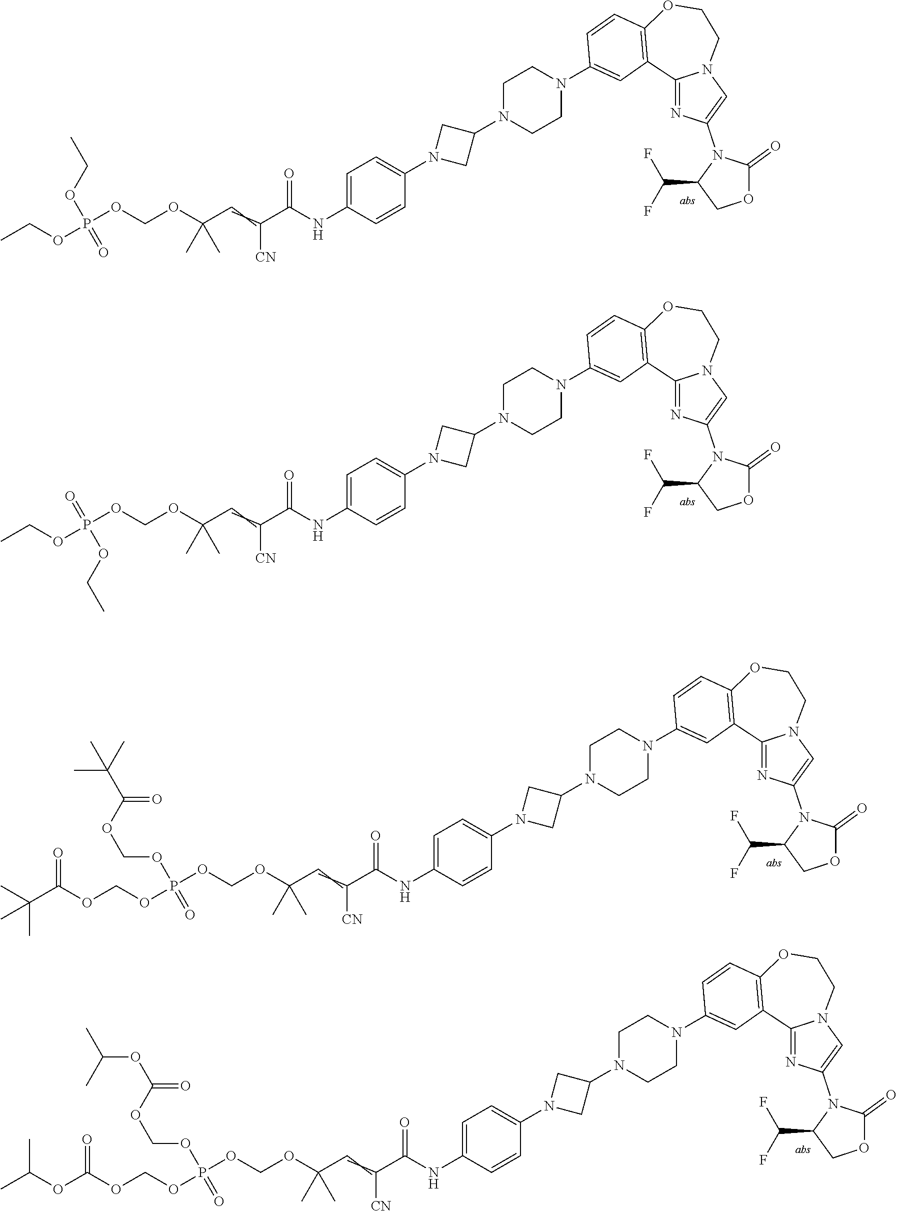 [SMILES: CCOP(=O)(OCC)OCOC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1 ; CCOP(=O)(OCC)OCOC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)OCOP(=O)(OCOC(=O)C(C)(C)C)OCOC(=O)C(C)(C)C ; CC(C)OC(=O)OCOP(=O)(OCOC(=O)OC(C)C)OCOC(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1]

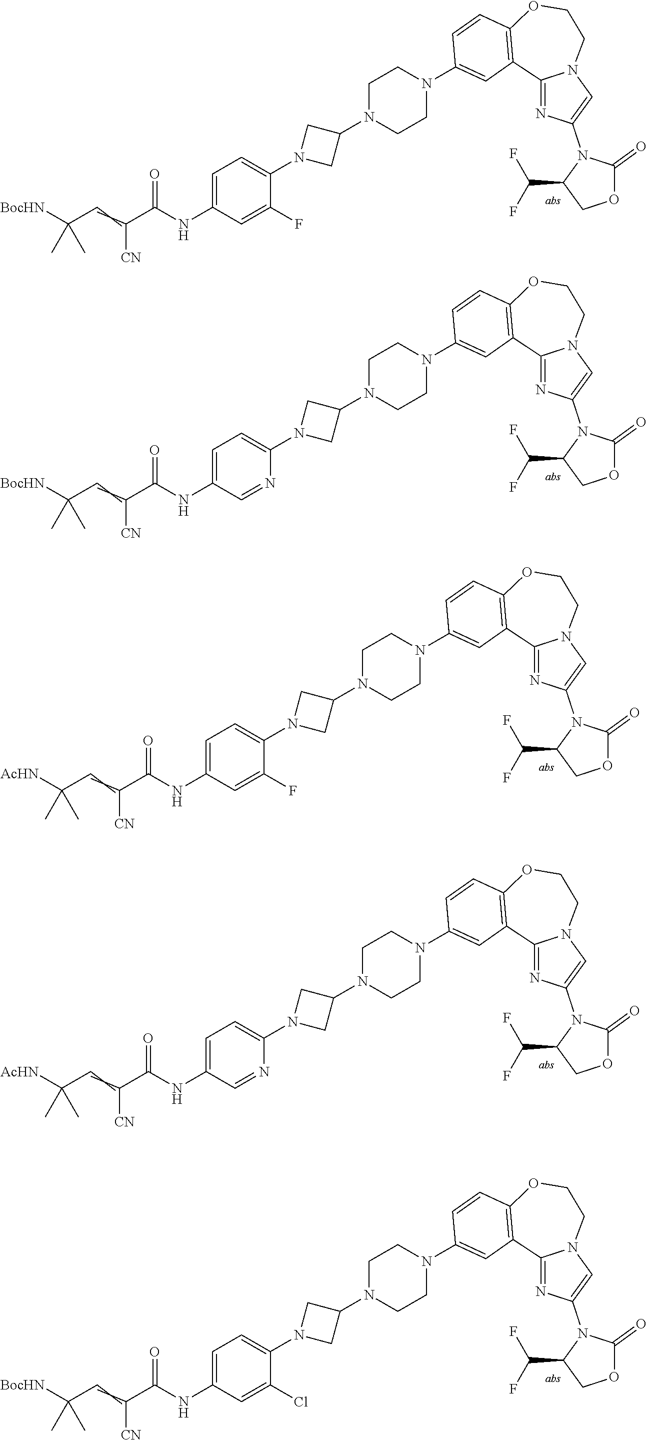

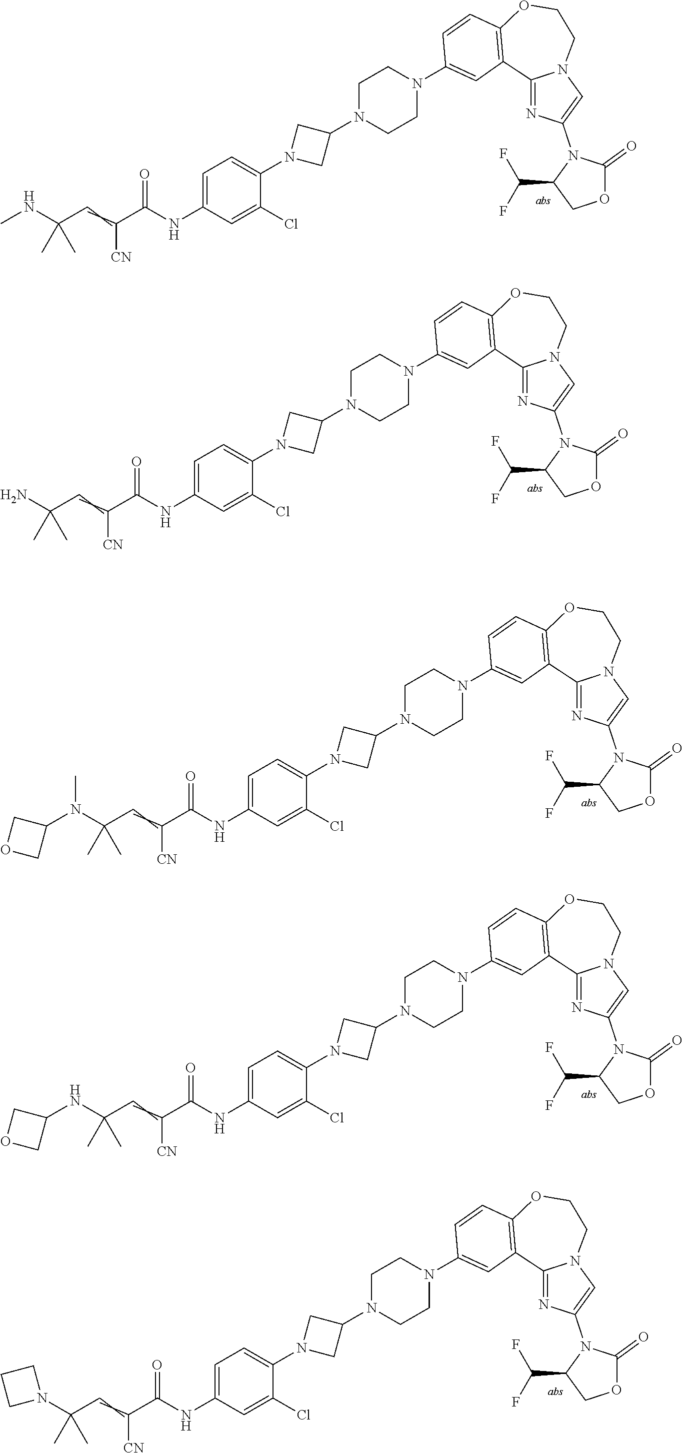

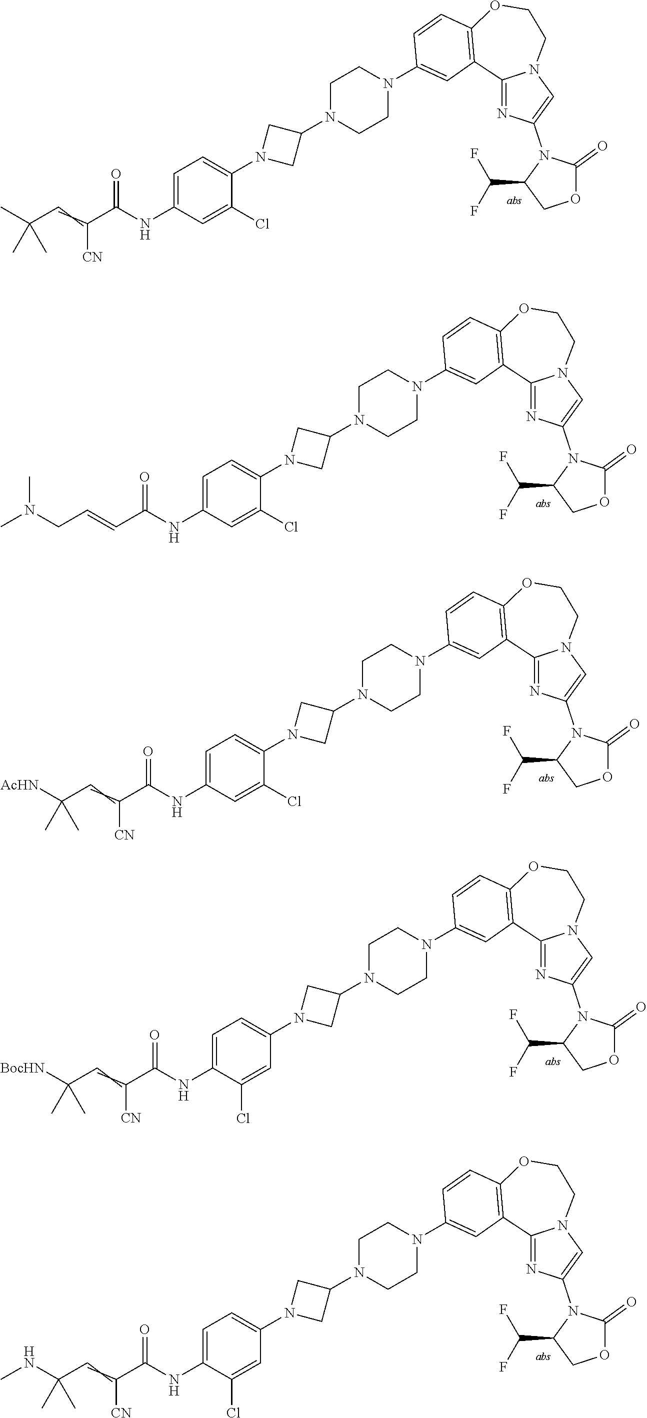

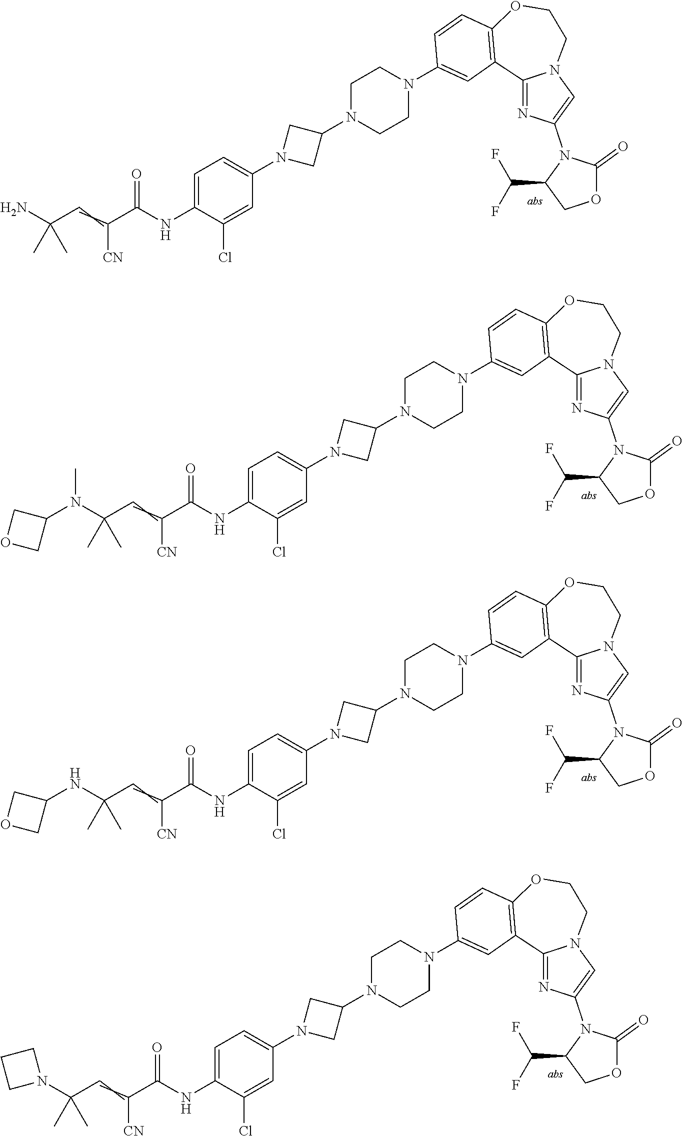 [SMILES: CC(C)(N)C=C(C#N)C(=O)Nc1ccc(N2CC(N3CCN(c4ccc5c(c4)-c4nc(N6C(=O)OC[C@H]6C(F)F)cn4CCO5)CC3)C2)cc1Cl ; CN(C1COC1)C(C)(C)C=C(C#N)C(=O)Nc1ccc(N2CC(N3CCN(c4ccc5c(c4)-c4nc(N6C(=O)OC[C@H]6C(F)F)cn4CCO5)CC3)C2)cc1Cl ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1Cl)NC1COC1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1Cl)N1CCC1]

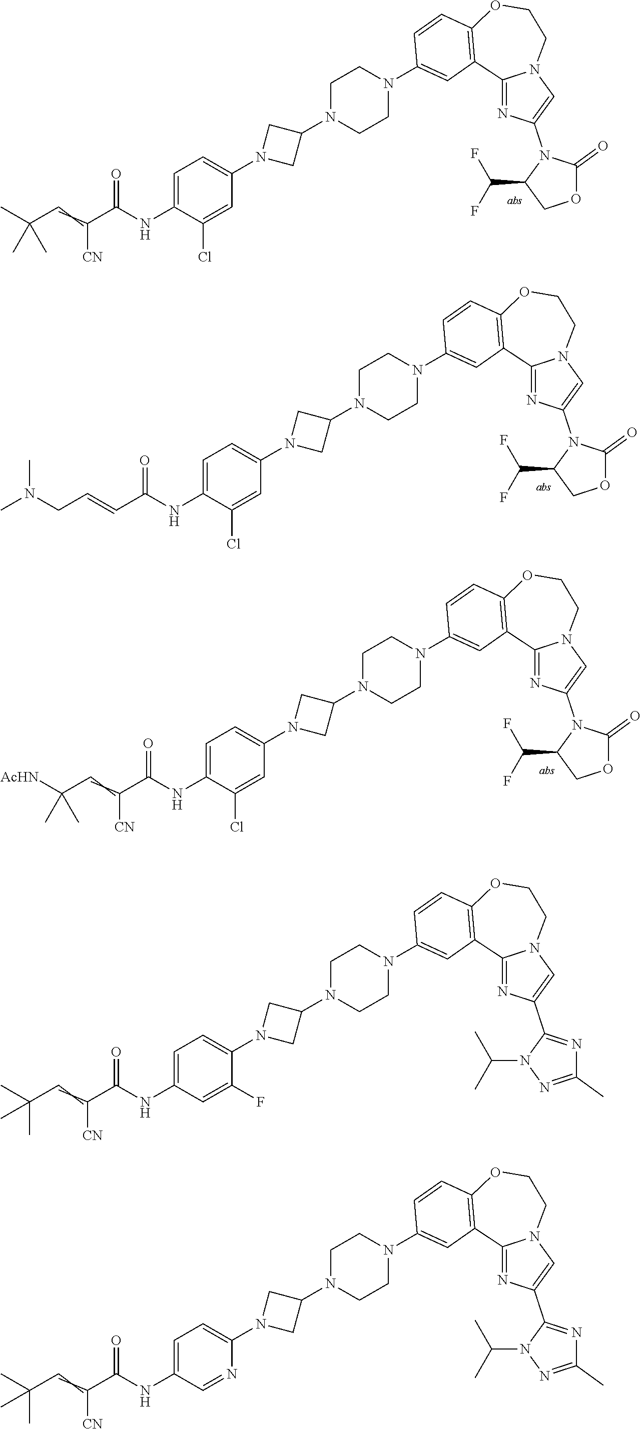

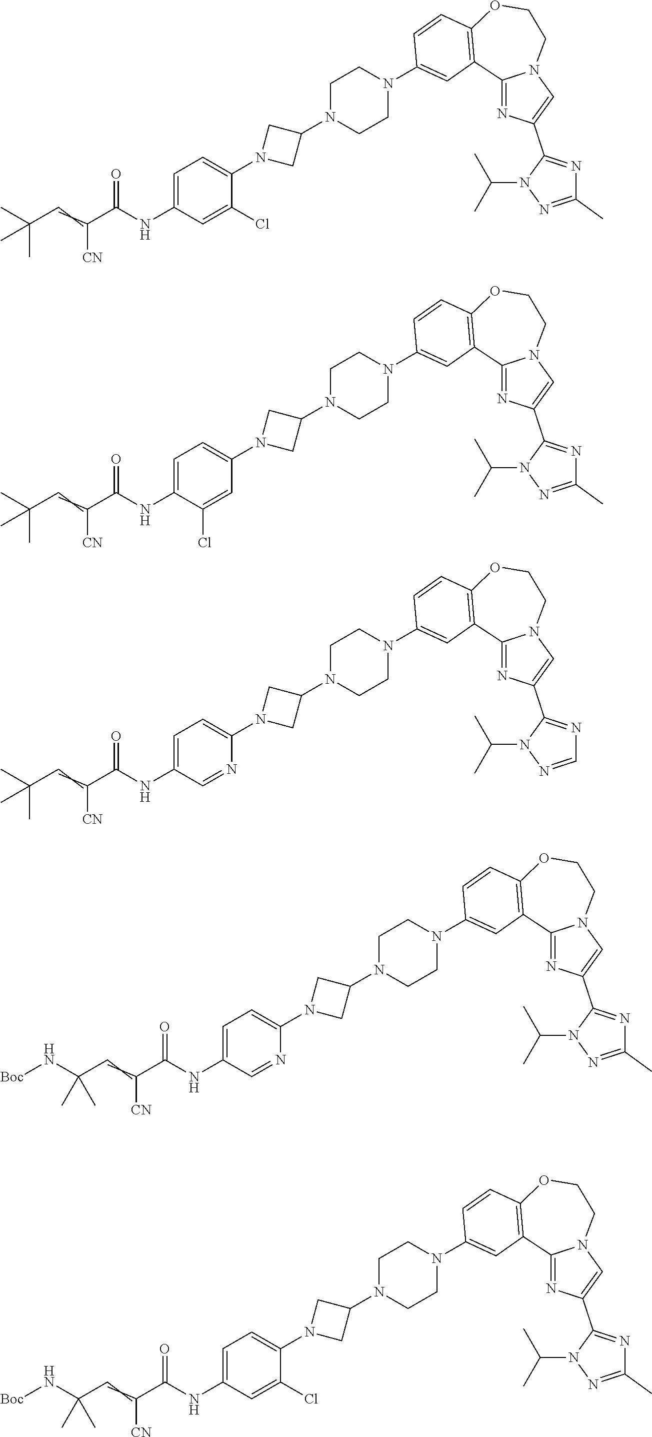

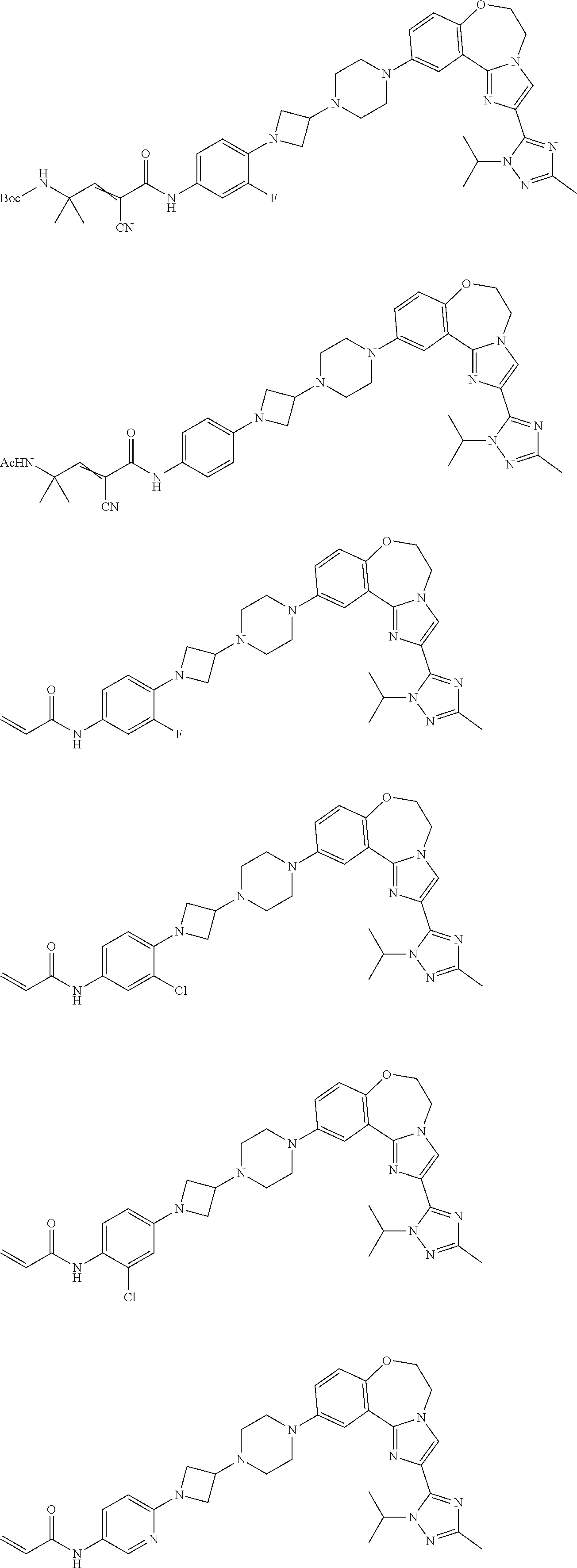

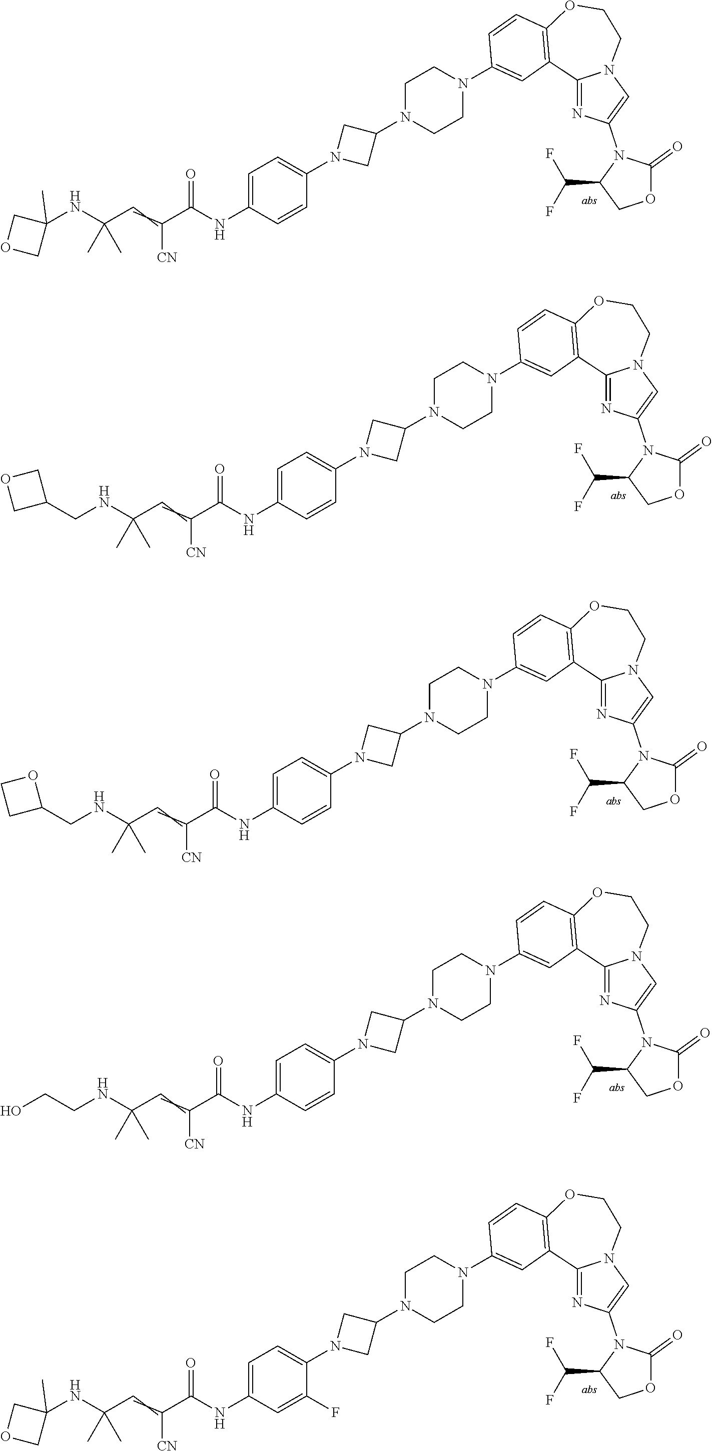

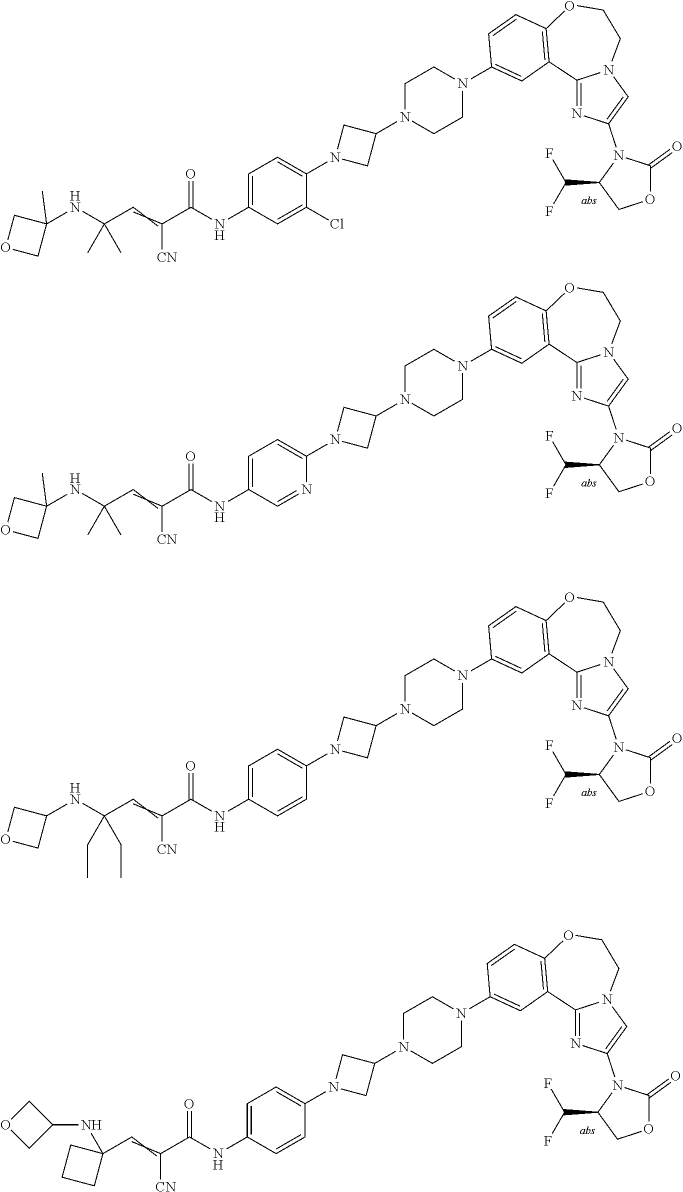 [SMILES: CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)c(Cl)c1)NC1(C)COC1 ; CC(C)(C=C(C#N)C(=O)Nc1ccc(N2CC(N3CCN(c4ccc5c(c4)-c4nc(N6C(=O)OC[C@H]6C(F)F)cn4CCO5)CC3)C2)nc1)NC1(C)COC1 ; CCC(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)(CC)NC1COC1 ; N#CC(=CC1(NC2COC2)CCC1)C(=O)Nc1ccc(N2CC(N3CCN(c4ccc5c(c4)-c4nc(N6C(=O)OC[C@H]6C(F)F)cn4CCO5)CC3)C2)cc1]

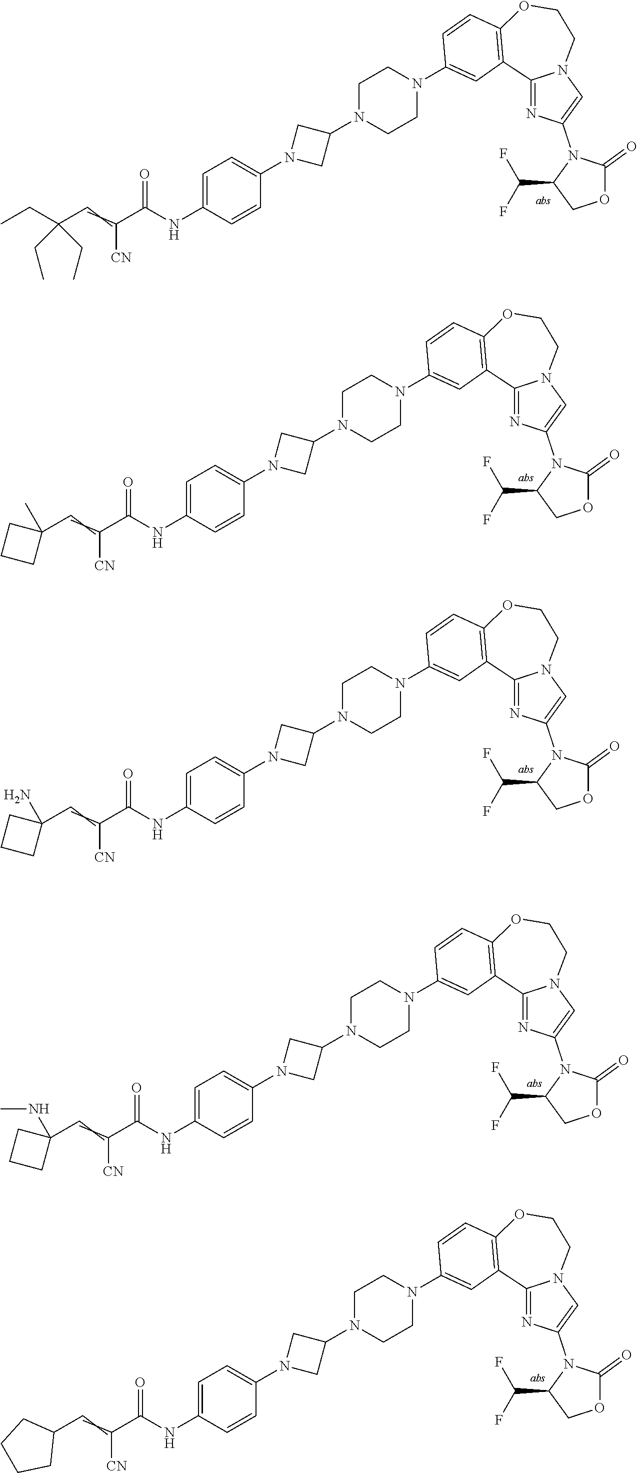 [SMILES: CCC(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)(CC)CC ; CC1(C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CCC1 ; N#CC(=CC1(N)CCC1)C(=O)Nc1ccc(N2CC(N3CCN(c4ccc5c(c4)-c4nc(N6C(=O)OC[C@H]6C(F)F)cn4CCO5)CC3)C2)cc1 ; CNC1(C=C(C#N)C(=O)Nc2ccc(N3CC(N4CCN(c5ccc6c(c5)-c5nc(N7C(=O)OC[C@H]7C(F)F)cn5CCO6)CC4)C3)cc2)CCC1 ; N#CC(=CC1CCCC1)C(=O)Nc1ccc(N2CC(N3CCN(c4ccc5c(c4)-c4nc(N6C(=O)OC[C@H]6C(F)F)cn4CCO5)CC3)C2)cc1]

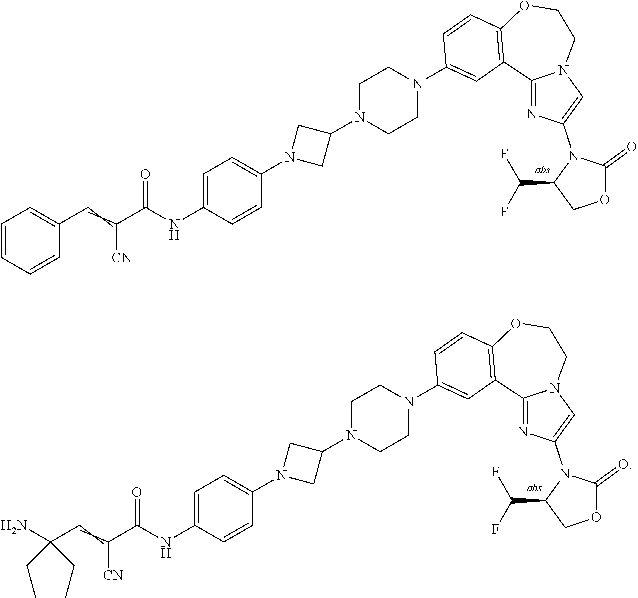 [SMILES: N#CC(=Cc1ccccc1)C(=O)Nc1ccc(N2CC(N3CCN(c4ccc5c(c4)-c4nc(N6C(=O)OC[C@H]6C(F)F)cn4CCO5)CC3)C2)cc1 ; CCC(N)(C=C(C#N)C(=O)Nc1ccc(N2CC(N3CCN(c4ccc5c(c4)-c4nc(N6C(=O)OC[C@H]6C(F)F)cn4CCO5)CC3)C2)cc1)CC]

19. A pharmaceutical composition, comprising a pharmaceutically acceptable excipient or carrier, and the compound or the isomer thereof, the crystalline form thereof, the pharmaceutically acceptable salt thereof, the hydrate thereof, or the solvate thereof according to claim 1 as an active ingredient.

20. Use of the compound or the isomer thereof, the crystalline form thereof, the pharmaceutically acceptable salt thereof, the hydrate thereof, or the solvate thereof according to claim 1 in preparing a medicament for treating a related disease mediated by PI3Kα.

21. (canceled)
